(19) United States
(12) Patent Application Publication
(10) Pub. No.: US 2026/0007684 A1
(43) Pub. Date: Jan. 8, 2026

(54) AMIDE DERIVATIVES FOR INHIBITING NLRP3 AND USES THEREOF

(71) Applicant: Ventus Therapeutics U.S., Inc., Waltham, MA (US)

(72) Inventors: Stéphane DORICH, Pointe-Claire (CA); Ramsay E. BEVERIDGE, Pointe-Claire (CA); Stéphane CIBLAT, Montreal (CA); Alexandre CÔTÉ, Laval (CA); Patrick CYR, Saint-Constant (CA); Miguel ST-ONGE, Saint-Lazare (CA)

(21) Appl. No.: 19/327,262

(22) Filed: Sep. 12, 2025

Related U.S. Application Data
(63) Continuation of application No. PCT/US2024/020220, filed on Mar. 15, 2024.
(60) Provisional application No. 63/490,968, filed on Mar. 17, 2023.
(60) Provisional application No. 63/519,074, filed on Aug. 11, 2023.

(51) Int. Cl. A61K 31/55; A61K 31/41; A61K 31/439; A61K 31/454; A61K 31/4545; C07D 401/04; C07D 401/14; C07D 403/04; C07D 471/08

(52) U.S. Cl. CPC A61K 31/55; A61K 31/41; A61K 31/439; A61K 31/454; A61K 31/4545; C07D 401/04; C07D 401/14; C07D 403/04; C07D 471/08

(57) ABSTRACT

The present disclosure relates to compounds of Formula (I-A):

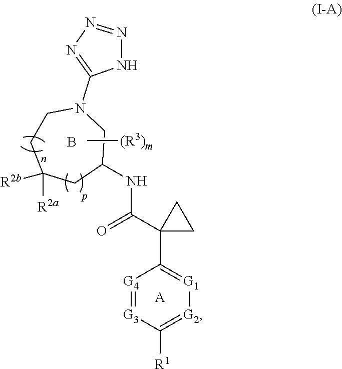

and pharmaceutically acceptable salts and tautomers thereof, wherein Ring A, Ring B, R¹, R^{2a}, R^{2b}, R³, m, n, and p are described herein, and methods of preparation, methods of treatment and prevention, and pharmaceutical compositions comprising same. The present disclosure further relates to the use of the compounds of Formula (I-A), and pharmaceutically acceptable salts and tautomers thereof, in the treatment and prevention of NLRP3-related diseases and disorders.

### RELATED APPLICATIONS

This application is a continuation of International Application No. PCT/US2024/020220, filed on Mar. 15, 2024, which claims priority to, and the benefit of, U.S. Provisional Patent Application No. 63/490,968, filed on Mar. 17, 2023, and U.S. Provisional Patent Application No. 63/519,074, filed on Aug. 11, 2023, the entire contents of each of which are incorporated herein by reference in their entireties.

### BACKGROUND

Innate immune responses are mediated by different types of receptors termed pattern-recognition receptors (PRRs). PRRs recognize the presence of pathogen-associated molecular patterns (PAMPs) and damage-associated molecular patterns (DAMPs). Once engaged these receptors trigger the activation of downstream inflammatory pathways that will help resolve injury. However, in many instances this activation can be uncontrolled and leads to disease.

The inflammasomes represent a class of PRRs that are crucial components of the innate immune response. Activation of the inflammasomes trigger a cascade of events that releases IL-1β, IL-18, and promotes an inflammatory form of cell death called pyroptosis induced by the activation of Gasdermin.

Pyroptosis is a unique form of inflammatory cell death that leads to the release of not only cytokines but also other intracellular components that promote a broader immune response both of the innate and acquired immune system. Thus, inflammasome activation is a major regulatory of the inflammatory cascade.

NLRP3 is the most characterized inflammasome and has been shown to be critical in innate immunity and inflammatory responses. While several other NLR complexes, such as NLRC4, are activated under very specific circumstances, NLRP3 can be activated by numerous stimuli and should be seen as a sensor of intracellular homeostatic imbalance. Therefore, its precise functioning is essential. In addition to playing a role in host immune defense, dysregulation of NLRP3 has been linked to the pathogenesis of many inflammatory disorders. These include genetic diseases such as cryopyrin-associated periodic syndromes (CAPS) which is caused by gain-of-function mutations in the NLRP3 gene, as well as many prevalent neurologic and systemic diseases. Importantly, NLRP3 hyperactivation has been demonstrated pre-clinically to play a critical role in a plethora of inflammatory and degenerative diseases including, NASH, atherosclerosis and other cardiovascular diseases, Alzheimer's disease, Parkinson's disease, diabetes, gout, and numerous other autoinflammatory diseases. See, e.g., Li et al., European Journal of Pharmacology (2022) 928:175091; Nguyen et al., Journal of Parkinson's Disease (2022) 12:2117-2133; Su et al., Current Medicinal Chemistry (2021) 28:569-582; Zahid et al., Frontiers in Immunology (2019) 10:2538. Thus, there is an unmet need in the field to develop small molecules for modulating NLRP3 activity to treat various diseases and disorders.

### SUMMARY

Provided herein are NLRP3 inhibitors of Formula (I-A):

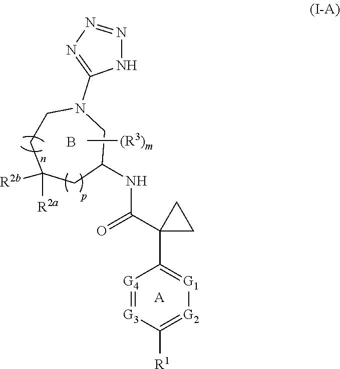

and pharmaceutically acceptable salts and tautomers thereof, wherein Ring A, Ring B, R¹, R^{2a}, R^{2b}, R³, m, n, and p are described herein.

Further provided are methods of preparation, methods of treatment and prevention, and pharmaceutical compositions comprising same.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972). Compounds described herein can additionally encompass individual isomers substantially free of other isomers, and alternatively, as mixtures of various isomers.

For example, compounds described herein may be referred to as “Rac-X”, which, for purposes of the Examples, including the data provided in the Assay Methods section, signifies a mixture of 2 or more stereoisomers, e.g., Compounds X′, X″, X″, X′″, XA, XB, XC, XD, XE, XF, XG, and/or XH. For purposes of claiming a “Rac-X” molecule, the claim may encompass a racemic composition of matter, but also may encompass an enantiomerically enriched composition of matter, e.g., enriched in one stereoisomer over others that may have been generated. For example, a claim may encompass a pharmaceutical composition comprising a “Rac-X” compound, or pharmaceutically acceptable salt thereof, and a pharmaceutically acceptable excipient, wherein the “Rac-X” compound is >80%, >85%, >90%, >95%, or >99% enantiomerically enriched.

Unless otherwise stated, compounds described herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₅, and C₅₋₆ alkyl.

“Alkyl” refers to a radical of a straight-chain or branched saturated hydrocarbon group having from 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆alkyl”). Examples of C₁₋₆ alkyl groups include methyl (—CH₃, C₁), ethyl (—CH₂CH₃, C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆).

“Haloalkyl” refers to a substituted alkyl group, as defined herein, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl group has 1 to 6 carbon atoms (“C₁₋₆ haloalkyl”). In some embodiments, the haloalkyl group has 1 to 5 carbon atoms (“C₁₋₅ haloalkyl”). In some embodiments, the haloalkyl group has 1 to 4 carbon atoms (“C₁₋₄ haloalkyl”). In some embodiments, the haloalkyl group has 1 to 3 carbon atoms (“C₁₋₃ haloalkyl”). In some embodiments, the haloalkyl group has 1 to 2 carbon atoms (“C₁₋₂ haloalkyl”). In some embodiments, all of the haloalkyl hydrogen atoms are replaced with fluoro to provide a perfluoroalkyl group. In some embodiments, all of the haloalkyl hydrogen atoms are replaced with chloro to provide a “perchloroalkyl” group. Examples of haloalkyl groups include —CF₃, —CF₂CF₃, —CF₂CF₂CF₃, —CCl₃, —CFCl₂, —CF₂Cl, and the like.

“Carbocyclyl” or “carbocyclic” refers to a radical of a non-aromatic cyclic hydrocarbon group having from 3 to 4 ring carbon atoms (“C₃₋₄ carbocyclyl”) and zero heteroatoms in the non-aromatic ring system. In some embodiments, a carbocyclyl group has 3 ring carbon atoms (“C₃ carbocyclyl”). In some embodiments, a carbocyclyl group has 4 ring carbon atoms (“C₄ carbocyclyl”). Exemplary C₃_4 carbocyclyl groups include, without limitation, cyclopropyl (C₃), cyclopropenyl (C₃), cyclobutyl (C₄), and cyclobutenyl (C₄).

“Heterocyclyl” or “heterocyclic” refers to a radical of a 3- to 4-membered non-aromatic ring system having ring carbon atoms and 1 ring heteroatom, wherein each heteroatom is independently selected from nitrogen, oxygen, and sulfur (“3-4 membered heterocyclyl”). In heterocyclyl groups that contain one nitrogen atom, the point of attachment can be a carbon or nitrogen atom, as valency permits.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl.

“Heteroaryl” refers to a radical of a 5-membered monocyclic aromatic ring system having ring carbon atoms and 1-2 ring heteroatoms provided in the aromatic ring system, wherein each ring heteroatom is independently selected from nitrogen, oxygen, and sulfur (“5-membered heteroaryl”). In heteroaryl groups that contain one or more nitrogen atoms, the point of attachment can be a carbon or nitrogen atom, as valency permits. Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl.

“Halo” or “halogen” refers to fluorine (fluoro, —F), chlorine (chloro, —Cl), bromine (bromo, —Br), or iodine (iodo, —I) radicals.

Affixing the suffix “-ene” to a group indicates the group is a divalent moiety, e.g., alkylene is the divalent moiety of alkyl, and haloalkylene is the divalent moiety of haloalkyl. By way of example, a C₁₋₃ alkylene, which may be linear or branched, include, but are not limited to, —CH₂—, —CH(CH₃)—, —C(CH₃)₂—, —CH₂CH₂—, —CH₂CH(CH₃)—, —CH₂C(CH₃)₂—, and —CH₂CH₂CH₂—.

An alkylene or haloalkylene “bridging group” refers to a group where the two ends of the divalent moiety are attached to different carbon atoms which are not vicinal (next to) each other. Exemplary bridging groups include methylene (—CH₂—), ethylene (—CH₂CH₂—), propylene (—CH₂CH₂CH₂—), and their corresponding halogenated (haloalkylene) groups. For clarity, the phrase “two R³ groups, together with the atoms to which they are attached, may be joined to form a C₁₋₃alkylene bridging group or C₁₋₃haloalkylene bridging group” is used interchangeably herein with the phrase “two R³ groups may be joined to form a C₁₋₃alkylene bridging group or C₁₋₃haloalkylene bridging group between the two atoms to which they are attached”; both phrases mean there are two non-vicinal R³ groups (attached to two carbon atoms of Ring B) which are joined to form a C₁₋₃alkylene bridging group or C₁₋₃haloalkylene bridging group on Ring B. An example of this bridging group is variable L of the compound of Formula (I-A-Bridge).

It is understood herein that compounds of Formula (I-A) and pharmaceutically acceptable salts thereof, each of which contain a terminal tetrazolyl group, may exist as a mixture of tautomeric isomers

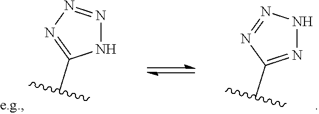 [SMILES: *c1nnn[nH]1 ; *c1nn[nH]n1]

Amino and oxygen protecting groups are described in detail in Protecting Groups in Organic Synthesis, T. W. Greene and P. G. M. Wuts, 3^{rd} edition, John Wiley & Sons, 1999.

Exemplary oxygen (hydroxyl) protecting groups include, but are not limited to, methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), tetrahydrofuranyl, benzyl (Bn), trimethylsilyl (TMS), triethylsilyl (TES), triisopropylsilyl (TIPS), t-butyldimethylsilyl (TBDMS), benzoylformate, acetate, chloroacetate, dichloroacetate, trichloroacetate, trifluoroacetate, methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), allyl carbonate, t-butyl carbonate (BOC), methanesulfonate (mesylate), benzylsulfonate, and tosylate (Ts).

Exemplary amino protecting groups include, but are not limited to, those that protect the amine as an amide, such as formamide, acetamide, chloroacetamide, trichloroacetamide, trifluoroacetamide, and phenylacetamide; protect the amine as a carbamate, such as methyl carbamate, ethyl carbamate, 9-fluorenylmethyl carbamate (Fmoc), t-butyl carbamate (BOC), 1-adamantyl carbamate (Adoc), and benzyl carbamate (Cbz); and protect the amine as a sulfonamide such as p-toluenesulfonamide (Ts), benzenesulfonamide, methanesulfonamide (Ms), and benzylsulfonamide.

Salts, pharmaceutically acceptable salts, and free bases of compounds of Formula (I-A) are contemplated herein.

“Salt” refers to any and all salts.

“Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Examples of pharmaceutically acceptable acid addition salts include, but are not limited to, salts formed from inorganic acids, such as hydrochloric acid, hydrobromic acid, phosphoric acid, sulfuric acid and perchloric acid salts, or salts formed from organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “free base” refers to a neutral non-ionized form of a compound which is not a salt or pharmaceutically acceptable salt.

A “patient” or “subject” is used interchangeably herein, and refers to a mammal, e.g., a human, mouse, rat, guinea pig, dog, cat, horse, cow, pig, or non-human primate, such as a monkey, chimpanzee, baboon, or rhesus. In certain embodiments, the patient or subject is human.

“Effective amount” or “therapeutically effective amount” are used interchangeably herein, and refer to an amount of a compound, or a pharmaceutically acceptable salt or tautomer thereof, sufficient to provide a therapeutic benefit in the treatment of a disease or disorder, or to delay or minimize one or more symptoms associated with the disease or disorder in a subject in need thereof. An effective amount can encompass an amount that improves overall therapy, reduces or avoids symptoms or causes of disease or disorder, or enhances the therapeutic efficacy of another therapeutic agent. The effective amount of a compound, or a pharmaceutically acceptable salt or tautomer thereof, may vary depending on such factors as the desired biological endpoint, the pharmacokinetics of the compound, the disease being treated, the mode of administration, and the age, health, and condition of the subject.

“Disease” or “disorder” are used interchangeably herein.

“Treating” or “treat” or “treatment” describes the management and care of a subject in need thereof, for the purpose of combating a disease or disorder in the subject, and includes the administration of a compound, or a pharmaceutically acceptable salt or tautomer thereof, as described herein, to alleviate the symptoms or complications of a disease or disorder, or to eliminate the disease or disorder. The term “treat” can also include treatment of a cell in vitro or treatment of an animal model (in vivo). It is to be appreciated that references to “treating” or “treatment” include the alleviation of established symptoms of a disease or disorder in a subject in need thereof, and therefore includes: (1) delaying the appearance of at least one clinical or subclinical symptom of the disease or disorder developing in a subject that is afflicted with the disease or disorder, (2) arresting, reducing or delaying the continued development of the disease or a relapse thereof in a subject (e.g., in case of maintenance treatment) or at least one clinical or subclinical symptom thereof, or (3) relieving or attenuating the disease in a subject, i.e., causing regression of the disease or disorder or at least one of its clinical or subclinical symptoms.

As used herein, the term “preventing,” “prevent,” or “protecting against” describes the management and care of a subject in need thereof that may have or has a predisposition for the disease or disorder but has not yet experienced or displayed symptoms or complications of a disease or disorder (e.g., clinical or subclinical symptoms of the disease or disorder), for the purpose of preventing the appearance of said symptoms or complications of the disease or disorder in the subject, and includes the administration of a compound, or a pharmaceutically acceptable salt or tautomer thereof, as described herein.

“Inhibition”, “inhibiting”, “inhibit” and “inhibitor”, and the like, refer to the ability of a compound, or a pharmaceutically acceptable salt or tautomer thereof, to reduce, slow, halt or prevent activity of a particular biological process (e.g., NLRP3 activity) in a cell relative to vehicle.

The phrase “at least one” refers to one instance or more than one instance.

The articles “a” and “an” are used in this disclosure to refer to one or more than one (i.e., to at least one) of the grammatical object of the article.

The term “and/or” is used in this disclosure to mean either “and” or “or” unless indicated otherwise.

### DETAILED DESCRIPTION OF SOME EMBODIMENTS

### (i) Compounds

Provided herein are compounds of Formula (I-A):

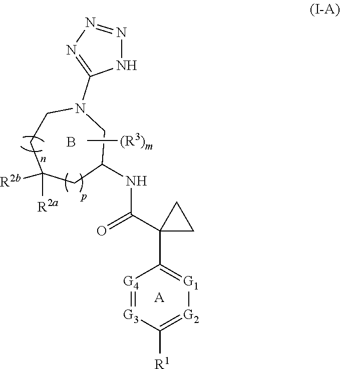

and pharmaceutically acceptable salts and tautomers thereof;
wherein:
Ring A is a ring system wherein:
   G₁ is CR^{G1} or N; G₂ is CR^{G2} or N; G₃ is CR^{G3} or N; and G₄ is CR^{G4} or N; provided no more than two of G₁, G₂, G₃, and G₄ are N;
   R¹ is halo, C₁₋₆ alkyl, C₁₋₆haloalkyl, —OR^{G5}, —SR^{G5}, —N(R^{G5})₂, C₃-C₄ carbocyclyl, or 3-4 membered heterocyclyl, wherein the carbocyclyl and heterocyclyl are independently substituted with 0, 1, 2, or 3 halo, C₁₋₆ alkyl, C₁₋₆ haloalkyl, —OR^{G5}, —SR^{G5}, or —N(R^{G5})₂,
   or R¹ and G₂, together with the atoms to which they are attached, are joined to form a 5-membered heteroaryl ring independently substituted with 0, 1, 2, or 3 R^{G7};
   R^{G1}, R^{G2}, R^{G3}, and R^{G4} are each independently selected from the group consisting of hydrogen, halo, C₁₋₆ alkyl, C₁₋₆ haloalkyl, and —OR^{G6};
   R^{G5} and R^{G6} are each independently hydrogen, C₁₋₆ alkyl, or C₁₋₆ haloalkyl; and
   each instance of R^{G7} is independently halo, C₁₋₆ alkyl, C₁₋₆ haloalkyl, —OR^{G5}, —SR^{G5}, and —N(R^{G5})₂;
and
Ring B is a ring system wherein:
   n is 0 or 1;
   p is 1 or 2;
   m is 0, 1, 2, or 3;
   each instance of R^{2a} and R^{2b} is independently hydrogen, halo, C₁₋₆ alkyl, C₁₋₆ haloalkyl, C₃-C₄ carbocyclyl, or 3-4 membered heterocyclyl, wherein the carbocyclyl or heterocyclyl are each independently substituted with 0, 1, 2, or 3 halo, or R^{2a} and R^{2b} are joined to form a C₃ carbocyclyl independently substituted with 0, 1, 2, or 3 halo; and
   each instance of R³ is independently halo, C₁₋₆ alkyl or C₁₋₆ haloalkyl, or two R³ groups are joined to form a C₁₋₃ alkylene bridging group or C₁₋₃ haloalkylene bridging group.

In some embodiments of Formula (I-A), the compound is of Formula:

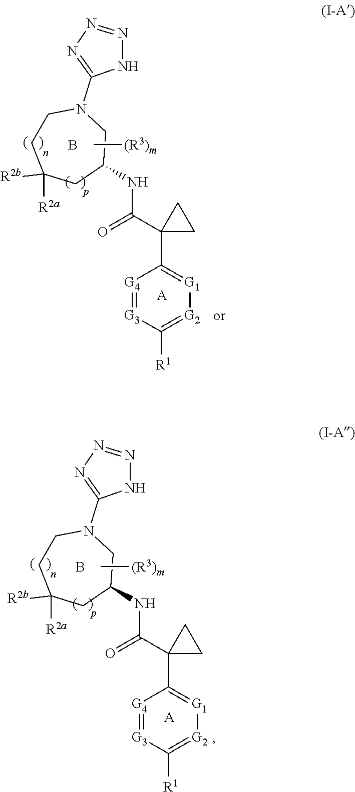

or a pharmaceutically acceptable salt or tautomer thereof.

In some embodiments of Formula (I-A), the compound is of Formula:

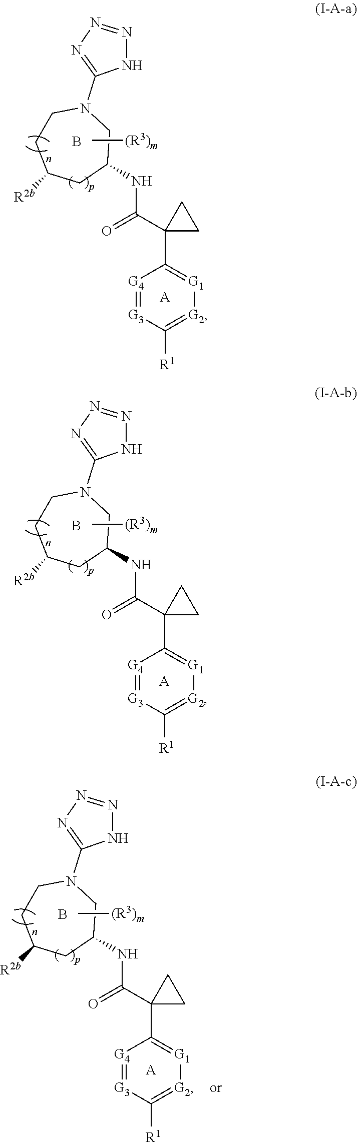

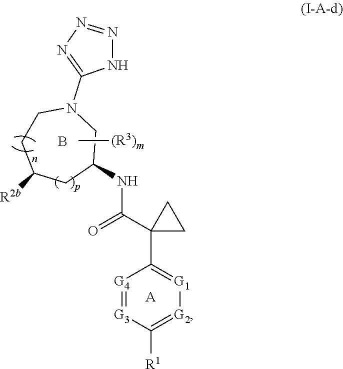

or a pharmaceutically acceptable salt or tautomer thereof.

In some embodiments of Formula (I-A), the compound is of Formula:

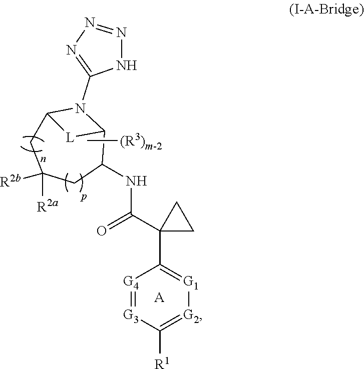

or a pharmaceutically acceptable salt or tautomer thereof, wherein L is a C₁₋₃ alkylene bridging group or a C₁₋₃ haloalkylene bridging group. In some embodiments, L is —CH₂CH₂—.

In some embodiments of Formula (I-A), the compound is of Formula:

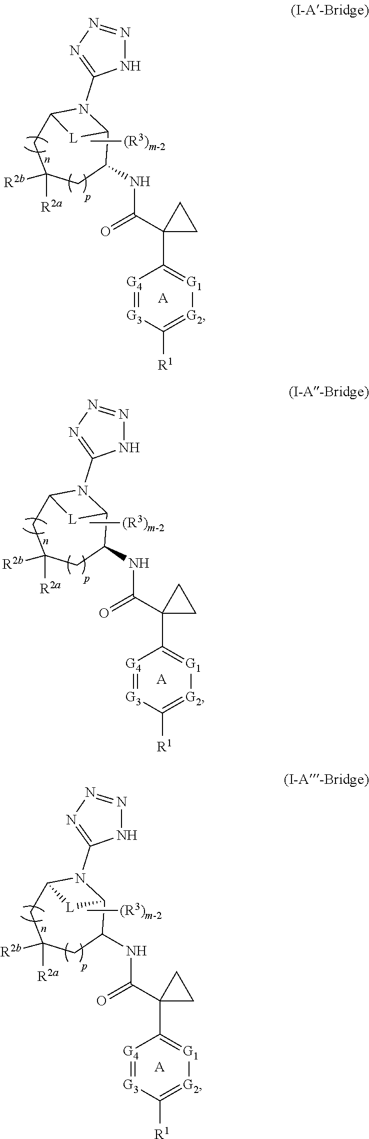

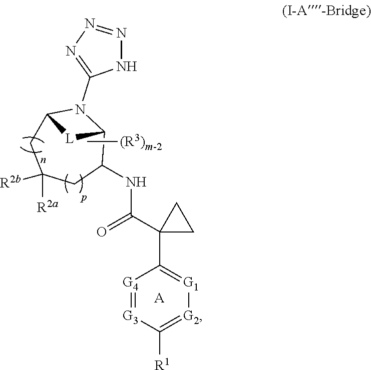

or a pharmaceutically acceptable salt or tautomer thereof, wherein L is a C₁₋₃ alkylene bridging group or a C₁₋₃ haloalkylene bridging group. In some embodiments, L is —CH₂CH₂—.

In some embodiments of Formula (I-A), the compound is of Formula:

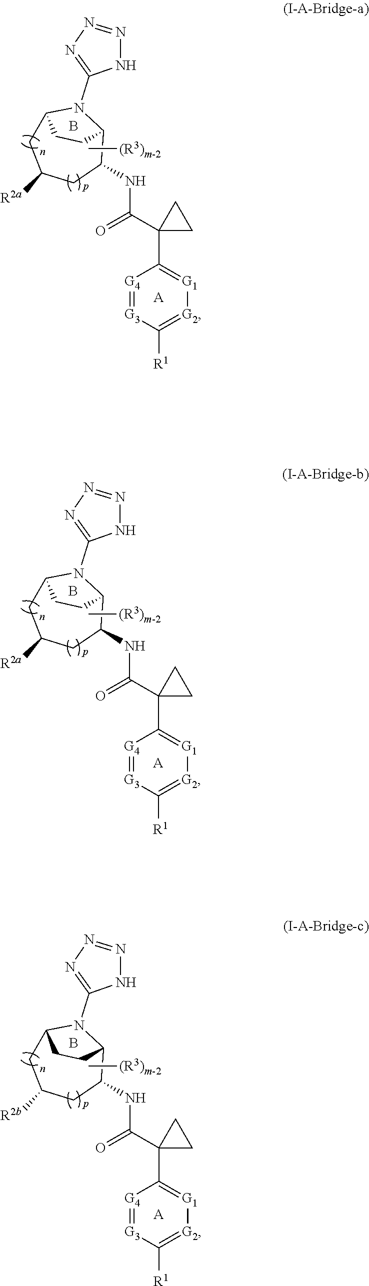

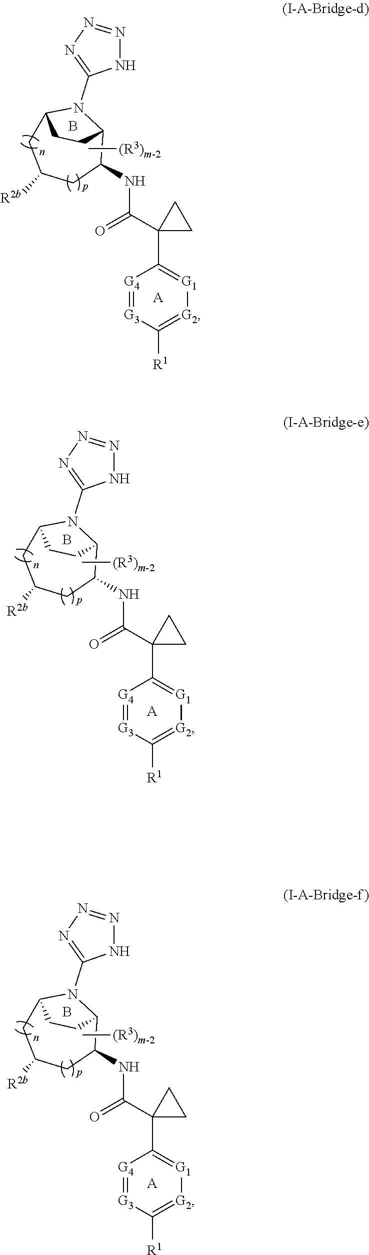

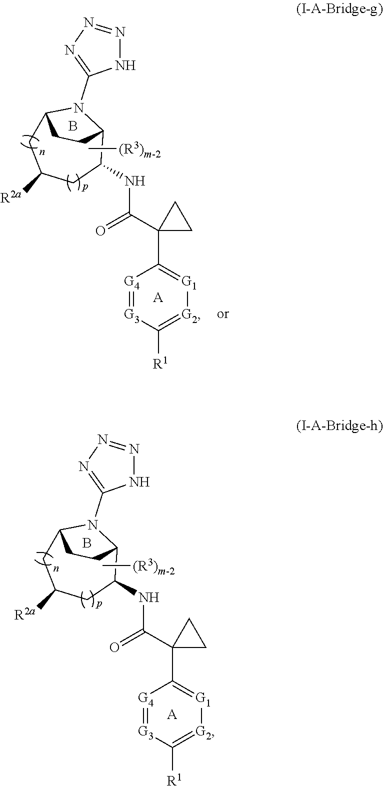

or a pharmaceutically acceptable salt or tautomer thereof.

Additional embodiments are further described below and herein.

(a) Ring A, G₁, G₂, G₃, G₄, R¹, R^{G1}, R^{G2}, R^{G3}, R^{G4}, R^{G5}, R^{G6}, and R^{G7}

As generally described herein, G₁ is CR^{G1} or N; G₂ is CR^{G2} or N; G₃ is CR^{G3} or N; and G₄ is CR^{G4} or N; provided no more than two of G₁, G₂, G₃, and G₄ are N.

In some embodiments, G₁ is CR^{G1}. In some embodiments, G₁ is N.

In some embodiments, G₂ is CR^{G2}. In some embodiments, G₂ is N.

In some embodiments, G₃ is CR^{G3}. In some embodiments, G₃ is N.

In some embodiments, G₄ is CR^{G4}. In some embodiments, G₄ is N.

In some embodiments, G₁ is CR^{G1}; G₂ is CR^{G2}; G₃ is CR^{G3}; and G₄ is CR^{G4}.

In some embodiments, at least one of G₁, G₂, G₃, and G₄ is N.

In some embodiments, G₁ is CR^{G1}; G₂ is CR^{G2}; G₃ is CR^{G3}; and G₄ is N. In some embodiments, G₁ is CR^{G1}; G₂ is CR^{G2}; G₃ is N; and G₄ is CR^{G4}. In some embodiments, G₁ is CR^{G1}; G₂ is N; G₃ is CR^{G3}; and G₄ is CR^{G4}. In some embodiments, G₁ is N; G₂ is CR^{G2}; G₃ is CR^{G3}; and G₄ is CR^{G4}.

In some embodiments, two of G₁, G₂, G₃, and G₄ is N.

For example, in some embodiments, G₁ is CR^{G1}; G₂ is CR^{G2}; G₃ is N; and G₄ is N. In some embodiments, G₁ is CR^{G1}; G₂ is N; G₃ is CR^{G3}; and G₄ is N. In some embodiments, G₁ is N; G₂ is CR^{G2}; G₃ is CR^{G3}; and G₄ is N. In some embodiments, G₁ is N; G₂ is N; G₃ is CR^{G3}; and G₄ is CR^{G4}. In some embodiments, G₁ is N; G₂ is CR^{G2}; G₃ is N; and G₄ is CR^{G4}. In some embodiments, G₁ is CR^{G1}; G₂ is N; G₃ is N; and G₄ is CR^{G4}.

In some embodiments, G₁ is CR^{G1}, G₂ is CR^{G2}, G₃ is CR^{G3}, and G₄ is CR^{G4}; G₁ is CR^{G1}, G₂ is CH, G₃ is CH, and G₄ is CH; G₁ is CR^{G1}, G₂ is CR^{G2}, G₃ is CH, and G₄ is CH; G₁ is CR^{G1}, G₂ is CH, G₃ is CR^{G3}, and G₄ is CH; G₁ is CR^{G1}, G₂ is N, G₃ is CR^{G3}, and G₄ is CR^{G4}; G₁ is CR^{G1}, G₂ is N, G₃ is CH, and G₄ is CH; G₁ is CR^{G}, G₂ is CR^{G2}, G₃ is N, and G₄ is CR^{G4}; G₁ is CR^{G}, G₂ is CH, G₃ is N, and G₄ is CH; G₁ is CR^{G1}, G₂ is CH, G₃ is N, and G₄ is CH; G₁ is CR^{G1}, G₂ is CR^{G2}, G₃ is CR^{G3}, and G₄ is N; or G₁ is CR^{G1}, G₂ is CH, G₃ is CH, and G₄ is N.

In some embodiments, G₁ is CH, G₂ is CH, G₃ is CH, and G₄ is CH; or G₁ is CH, G₂ is CR^{G2}, G₃ is CH, and G₄ is CH.

As generally described herein, R¹ is halo, C₁₋₆alkyl, C₁₋₆haloalkyl, —OR^{G5}, —SR^{G5}, —N(R^{G5})₂, C₃-C₄ carbocyclyl, or 3-4 membered heterocyclyl, wherein the carbocyclyl and heterocyclyl are independently substituted with 0, 1, 2, or 3 halo, C₁₋₆ alkyl, C₁₋₆ haloalkyl, —OR^{G5}, —SR^{G5}, or —N(R^{G5})₂, or R¹ and G₂, together with the atoms to which they are attached, are joined to form a 5-membered heteroaryl ring independently substituted with 0, 1, 2, or 3 R^{G7} groups selected from the group consisting of halo, C₁₋₆ alkyl, C₁₋₆haloalkyl, —OR^{G5}, —SR^{G5}, and —N(R^{G5})₂.

In some embodiments, R¹ is halo.

In some embodiments, R¹ is F, Cl, Br, or I. In some embodiments, R¹ is F, Cl, or Br. In some embodiments, R¹ is F or Cl.

In some embodiments, R¹ is F. In some embodiments, R¹ is Cl. In some embodiments, R¹ is Br. In some embodiments, R¹ is I.

In some embodiments, R¹ is C₁₋₆ alkyl independently substituted with 0, 1, 2, or 3 halo, C₁₋₆ alkyl, C₁₋₆haloalkyl, —OR^{G5}, —SR^{G5}, or —N(R^{G5})₂.

In some embodiments, R¹ is C₁₋₆ alkyl independently substituted with 1, 2, or 3 halo, C₁₋₆ alkyl, C₁₋₆haloalkyl, —OR^{G5}, —SR^{G5}, or —N(R^{G5})₂.

In some embodiments, R¹ is unsubstituted C₁₋₆ alkyl.

In some embodiments, R¹ is methyl. In some embodiments, R¹ is ethyl. In some embodiments, R¹ is propyl. In some embodiments, R¹ is butyl. In some embodiments, R¹ is pentyl. In some embodiments, R¹ is hexyl. In some embodiments, R¹ is isopropyl. In some embodiments, R¹ is isobutyl. In some embodiments, R¹ is isopentyl. In some embodiments, R¹ is isohexyl. In some embodiments, R¹ is secbutyl. In some embodiments, R¹ is secpentyl. In some embodiments, R¹ is sechexyl. In some embodiments, R¹ is tertbutyl.

In some embodiments, R¹ is C₁₋₆ haloalkyl.

In some embodiments, R¹ is halomethyl. In some embodiments, R¹ is haloethyl. In some embodiments, R¹ is halopropyl. In some embodiments, R¹ is halobutyl. In some embodiments, R¹ is halopentyl. In some embodiments, R¹ is halohexyl.

In some embodiments, R¹ is —OR^{G5}.

In some embodiments, R¹ is —SR^{G5}.

In some embodiments, R¹ is —N(R^{G5})₂.

In some embodiments, R¹ is C₃-C₄ carbocyclyl or 3-4 membered heterocyclyl, wherein the carbocyclyl or heterocyclyl are independently substituted with 0, 1, 2, or 3 halo, C₁₋₆ alkyl, C₁₋₆ haloalkyl, —OR^{G5}, —SR^{G5}, or —N(R^{G5})₂.

In some embodiments, R¹ is C₃-C₄ carbocyclyl independently substituted with 0, 1, 2, or 3 halo, C₁₋₆alkyl, C₁₋₆haloalkyl, —OR^{G5}, —SR^{G5}, or —N(R^{G5})₂.

In some embodiments, R¹ is C₃ carbocyclyl independently substituted with 0, 1, 2, or 3 halo, C₁₋₆ alkyl, C₁₋₆haloalkyl, —OR^{G5}, —SR^{G5}, or —N(R^{G5})₂.

In some embodiments, R¹ is C₄ carbocyclyl independently substituted with 0, 1, 2, or 3 halo, C₁₋₆ alkyl, C₁₋₆haloalkyl, —OR^{G5}, —SR^{G5}, or —N(R^{G5})₂.

In some embodiments, R¹ is C₃-C₄ carbocyclyl independently substituted with 1, 2, or 3 halo, C₁₋₆ alkyl, C₁₋₆haloalkyl, —OR^{G5}, —SR^{G5}, or —N(R^{G5})₂.

In some embodiments, R¹ is C₃ carbocyclyl independently substituted with 1, 2, or 3 halo, C₁₋₆ alkyl, C₁₋₆haloalkyl, —OR^{G5}, —SR^{G5}, or —N(R^{G5})₂.

In some embodiments, R¹ is C₄ carbocyclyl independently substituted with 1, 2, or 3 halo, C₁₋₆ alkyl, C₁₋₆haloalkyl, —OR^{G5}, —SR^{G5}, or —N(R^{G5})₂.

In some embodiments, R¹ is unsubstituted C₃-C₄ carbocyclyl.

In some embodiments, R¹ is unsubstituted C₃ carbocyclyl. In some embodiments, R¹ is unsubstituted C₄ carbocyclyl.

In some embodiments, R¹ is C₃-C₄ carbocyclyl substituted with 1 halo, C₁₋₆alkyl, C₁₋₆haloalkyl, —OR^{G5}, —SR^{G5}, or —N(R^{G5})₂.

In some embodiments, R¹ is C₃ carbocyclyl substituted with 1 halo, C₁₋₆ alkyl, C₁₋₆ haloalkyl, —OR^{G5}, —SR^{G5}, or —N(R^{G5})₂. In some embodiments, R¹ is C₄ carbocyclyl substituted with 1 halo, C₁₋₆ alkyl, C₁₋₆haloalkyl, —OR^{G5}, —SR^{G5}, or —N(R^{G5})₂.

In some embodiments, R¹ is C₃-C₄ carbocyclyl independently substituted with 2 halo, C₁₋₆ alkyl, C₁₋₆haloalkyl, —OR^{G5}, —SR^{G5}, or —N(R^{G5})₂.

In some embodiments, R¹ is C₃ carbocyclyl independently substituted with 2 halo, C₁₋₆ alkyl, C₁₋₆ haloalkyl, —OR^{G5}, —SR^{G5}, or —N(R^{G5})₂. In some embodiments, R¹ is C₄ carbocyclyl independently substituted with 2 halo, C₁₋₆ alkyl, C₁₋₆ haloalkyl, —OR^{G5}, —SR^{G5}, or —N(R^{G5})₂.

In some embodiments, R¹ is C₃-C₄ carbocyclyl independently substituted with 3 halo, C₁₋₆ alkyl, C₁₋₆haloalkyl, —OR^{G5}, —SR^{G5}, or —N(R^{G5})₂.

In some embodiments, R¹ is C₃ carbocyclyl independently substituted with 3 halo, C₁₋₆ alkyl, C₁₋₆ haloalkyl, —OR^{G5}, —SR^{G5}, or —N(R^{G5})₂. In some embodiments, R¹ is C₄ carbocyclyl independently substituted with 3 halo, C₁₋₆ alkyl, C₁₋₆ haloalkyl, —OR^{G5}, —SR^{G5}, or —N(R^{G5})₂.

In some embodiments, R¹ is C₃-C₄ carbocyclyl independently substituted with 1, 2, or 3 halo, C₁₋₆ alkyl, C₁₋₆haloalkyl, —OR^{G5}, —SR^{G5}, or —N(R^{G5})₂, provided at least one substituent is halo.

In some embodiments, R¹ is C₃ carbocyclyl substituted with at least one halo. In some embodiments, R¹ is C₄ carbocyclyl substituted with at least one halo.

In some embodiments, R¹ is C₃-C₄ carbocyclyl substituted with at least one of F, Cl, Br, or I. In some embodiments, R¹ is C₃-C₄ carbocyclyl substituted with at least one of F, Cl, or Br. In some embodiments, R¹ is C₃-C₄ carbocyclyl substituted with at least one of F or Cl.

In some embodiments, R¹ is C₃ carbocyclyl substituted with at least one of F, Cl, Br, or I. In some embodiments, R¹ is C₃ carbocyclyl substituted with at least one of F, Cl, or Br. In some embodiments, R¹ is C₃ carbocyclyl substituted with at least one of F or Cl.

In some embodiments, R¹ is C₄ carbocyclyl substituted with at least one of F, Cl, Br, or I. In some embodiments, R¹ is C₄ carbocyclyl substituted with at least one of F, Cl, or Br. In some embodiments, R¹ is C₄ carbocyclyl substituted with at least one of F or Cl.

In some embodiments, R¹ is C₃-C₄ carbocyclyl substituted with at least one F. In some embodiments, R¹ is C₃-C₄ carbocyclyl substituted with at least one Cl. In some embodiments, R¹ is C₃-C₄ carbocyclyl substituted with at least one Br. In some embodiments, R¹ is C₃-C₄ carbocyclyl substituted with at least one I.

In some embodiments, R¹ is C₃ carbocyclyl substituted with at least one F. In some embodiments, R¹ is C₃ carbocyclyl substituted with at least one Cl. In some embodiments, R¹ is C₃ carbocyclyl substituted with at least one Br. In some embodiments, R¹ is C₃ carbocyclyl substituted with at least one I.

In some embodiments, R¹ is C₄ carbocyclyl substituted with at least one F. In some embodiments, R¹ is C₄ carbocyclyl substituted with at least one Cl. In some embodiments, R¹ is C₄ carbocyclyl substituted with at least one Br. In some embodiments, R¹ is C₄ carbocyclyl substituted with at least one I.

In some embodiments, R¹ is C₃-C₄ carbocyclyl substituted with at least one C₁₋₆ alkyl.

In some embodiments, R¹ is C₃-C₄ carbocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is C₁₋₆ alkyl.

In some embodiments, R¹ is C₃-C₄ carbocyclyl substituted with at least one methyl. In some embodiments, R¹ is C₃-C₄ carbocyclyl substituted with at least one ethyl. In some embodiments, R¹ is C₃-C₄ carbocyclyl substituted with at least one propyl. In some embodiments, R¹ is C₃-C₄ carbocyclyl substituted with at least one butyl. In some embodiments, R¹ is C₃-C₄ carbocyclyl substituted with at least one pentyl. In some embodiments, R¹ is C₃-C₄ carbocyclyl substituted with at least one hexyl. In some embodiments, R¹ is C₃-C₄ carbocyclyl substituted with at least one isopropyl. In some embodiments, R¹ is C₃-C₄ carbocyclyl substituted with at least one isobutyl. In some embodiments, R¹ is C₃-C₄ carbocyclyl substituted with at least one isopentyl. In some embodiments, R¹ is C₃-C₄ carbocyclyl substituted with at least one isohexyl. In some embodiments, R¹ is C₃-C₄ carbocyclyl substituted with at least one secbutyl. In some embodiments, R¹ is C₃-C₄ carbocyclyl substituted with at least one secpentyl. In some embodiments, R¹ is C₃-C₄ carbocyclyl substituted with at least one sechexyl. In some embodiments, R¹ is C₃-C₄ carbocyclyl substituted with at least one tertbutyl.

In some embodiments, R¹ is C₃ carbocyclyl substituted with at least one methyl. In some embodiments, R¹ is C₃ carbocyclyl substituted with at least one ethyl. In some embodiments, R¹ is C₃ carbocyclyl substituted with at least one propyl. In some embodiments, R¹ is C₃ carbocyclyl substituted with at least one butyl. In some embodiments, R¹ is C₃ carbocyclyl substituted with at least one pentyl. In some embodiments, R¹ is C₃ carbocyclyl substituted with at least one hexyl. In some embodiments, R¹ is C₃ carbocyclyl substituted with at least one isopropyl. In some embodiments, R¹ is C₃ carbocyclyl substituted with at least one isobutyl. In some embodiments, R¹ is C₃ carbocyclyl substituted with at least one isopentyl. In some embodiments, R¹ is C₃ carbocyclyl substituted with at least one isohexyl. In some embodiments, R¹ is C₃ carbocyclyl substituted with at least one secbutyl. In some embodiments, R¹ is C₃ carbocyclyl substituted with at least one secpentyl. In some embodiments, R¹ is C₃ carbocyclyl substituted with at least one sechexyl. In some embodiments, R¹ is C₃ carbocyclyl substituted with at least one tertbutyl.

In some embodiments, R¹ is C₄ carbocyclyl substituted with at least one methyl. In some embodiments, R¹ is C₄ carbocyclyl substituted with at least one ethyl. In some embodiments, R¹ is C₄ carbocyclyl substituted with at least one propyl. In some embodiments, R¹ is C₄ carbocyclyl substituted with at least one butyl. In some embodiments, R¹ is C₄ carbocyclyl substituted with at least one pentyl. In some embodiments, R¹ is C₄ carbocyclyl substituted with at least one hexyl. In some embodiments, R¹ is C₄ carbocyclyl substituted with at least one isopropyl. In some embodiments, R¹ is C₄ carbocyclyl substituted with at least one isobutyl. In some embodiments, R¹ is C₄ carbocyclyl substituted with at least one isopentyl. In some embodiments, R¹ is C₄ carbocyclyl substituted with at least one isohexyl. In some embodiments, R¹ is C₄ carbocyclyl substituted with at least one secbutyl. In some embodiments, R¹ is C₄ carbocyclyl substituted with at least one secpentyl. In some embodiments, R¹ is C₄ carbocyclyl substituted with at least one sechexyl. In some embodiments, R¹ is C₄ carbocyclyl substituted with at least one tertbutyl.

In some embodiments, R¹ is C₃-C₄ carbocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is C₁₋₆ haloalkyl.

In some embodiments, R¹ is C₃ carbocyclyl substituted with at least one C₁₋₆ haloalkyl. In some embodiments, R¹ is C₄ carbocyclyl substituted with at least one C₁₋₆ haloalkyl.

In some embodiments, R¹ is C₃-C₄ carbocyclyl substituted with at least one halomethyl. In some embodiments, R¹ is C₃-C₄ carbocyclyl substituted with at least one haloethyl. In some embodiments, R¹ is C₃-C₄ carbocyclyl substituted with at least one halopropyl. In some embodiments, R¹ is C₃-C₄ carbocyclyl substituted with at least one halobutyl. In some embodiments, R¹ is C₃-C₄ carbocyclyl substituted with at least one halopentyl. In some embodiments, R¹ is C₃-C₄ carbocyclyl substituted with at least one halohexyl.

In some embodiments, R¹ is C₃ carbocyclyl substituted with at least one halomethyl. In some embodiments, R¹ is C₃ carbocyclyl substituted with at least one haloethyl. In some embodiments, R¹ is C₃ carbocyclyl substituted with at least one halopropyl. In some embodiments, R¹ is C₃ carbocyclyl substituted with at least one halobutyl. In some embodiments, R¹ is C₃ carbocyclyl substituted with at least one halopentyl. In some embodiments, R¹ is C₃ carbocyclyl substituted with at least one halohexyl.

In some embodiments, R¹ is C₄ carbocyclyl substituted with at least one halomethyl. In some embodiments, R¹ is C₄ carbocyclyl substituted with at least one haloethyl. In some embodiments, R¹ is C₄ carbocyclyl substituted with at least one halopropyl. In some embodiments, R¹ is C₄ carbocyclyl substituted with at least one halobutyl. In some embodiments, R¹ is C₄ carbocyclyl substituted with at least one halopentyl. In some embodiments, R¹ is C₄ carbocyclyl substituted with at least one halohexyl.

In some embodiments, R¹ is C₃-C₄ carbocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is —OR^{G5}.

In some embodiments, R¹ is C₃ carbocyclyl substituted with at least one —OR^{G5}. In some embodiments, R¹ is C₄ carbocyclyl substituted with at least one —OR^{G5}.

In some embodiments, R¹ is C₃-C₄ carbocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is —SR^{G5}.

In some embodiments, R¹ is C₃ carbocyclyl substituted with at least one —SR^{G5}. In some embodiments, R¹ is C₄ carbocyclyl substituted with at least one —SR^{G5}.

In some embodiments, R¹ is C₃-C₄ carbocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is —N(R^{G5})₂.

In some embodiments, R¹ is C₃ carbocyclyl substituted with at least one —N(R^{G5})₂. In some embodiments, R¹ is C₄ carbocyclyl substituted with at least one —N(R^{G5})₂.

In some embodiments, R¹ is 3-4 membered heterocyclyl independently substituted with 0, 1, 2, or 3 halo, C₁₋₆alkyl, C₁₋₆haloalkyl, —OR^{G5}, —SR^{G5}, or —N(R^{G5})₂.

In some embodiments, R¹ is 3-membered heterocyclyl independently substituted with 0, 1, 2, or 3 halo, C₁₋₆alkyl, C₁₋₆haloalkyl, —OR^{G5}, —SR^{G5}, or —N(R^{G5})₂. In some embodiments, R¹ is 4-membered heterocyclyl independently substituted with 0, 1, 2, or 3 halo, C₁₋₆ alkyl, C₁₋₆ haloalkyl, —OR^{G5}, —SR^{G5} or —N(R^{G5})₂.

In some embodiments, R¹ is 3-4 membered heterocyclyl independently substituted with 1, 2, or 3 halo, C₁₋₆alkyl, C₁₋₆haloalkyl, —OR^{G5}, —SR^{G5}, or —N(R^{G5})₂.

In some embodiments, R¹ is 3-membered heterocyclyl independently substituted with 1, 2, or 3 halo, C₁₋₆alkyl, C₁₋₆haloalkyl, —OR^{G}, —SR^{G}, or —N(R^{G})₂. In some embodiments, R¹ is 4-membered heterocyclyl independently substituted with 1, 2, or 3 halo, C₁₋₆ alkyl, C₁₋₆ haloalkyl, —OR^{G5}, —SR^{G5} or —N(R^{G5})₂.

In some embodiments, R¹ is unsubstituted 3-4 membered heterocyclyl.

In some embodiments, R¹ is unsubstituted 3-membered heterocyclyl. In some embodiments, R¹ is unsubstituted 4-membered heterocyclyl.

In some embodiments, R¹ is 3-4 membered heterocyclyl independently substituted with 1 halo, C₁₋₆alkyl, C₁₋₆haloalkyl, —OR^{G5}, —SR^{G5}, or —N(R^{G5})₂.

In some embodiments, R¹ is 3-membered heterocyclyl substituted with 1 halo, C₁₋₆ alkyl, C₁₋₆ haloalkyl, —OR^{G5}, —SR^{G5}, or —N(R^{G5})₂. In some embodiments, R¹ is 4-membered heterocyclyl substituted with 1 halo, C₁₋₆alkyl, C₁₋₆haloalkyl, —OR^{G5}, —SR^{G5}, or —N(R^{G5})₂.

In some embodiments, R¹ is 3-4 membered heterocyclyl independently substituted with 2 halo, C₁₋₆alkyl, C₁₋₆haloalkyl, —OR^{G5}, —SR^{G5}, or —N(R^{G5})₂.

In some embodiments, R¹ is 3-membered heterocyclyl independently substituted with 2 halo, C₁₋₆ alkyl, C₁₋₆ haloalkyl, —OR^{G5}, —SR^{G5}, or —N(R^{G5})₂. In some embodiments, R¹ is 4-membered heterocyclyl independently substituted with 2 halo, C₁₋₆ alkyl, C₁₋₆ haloalkyl, —OR^{G5}, —SR^{G5}, or —N(R^{G5})₂.

In some embodiments, R¹ is 3-4 membered heterocyclyl independently substituted with 3 halo, C₁₋₆alkyl, C₁₋₆haloalkyl, —OR^{G5}, —SR^{G5}, or —N(R^{G5})₂.

In some embodiments, R¹ is 3-membered heterocyclyl independently substituted with 3 halo, C₁₋₆ alkyl, C₁₋₆ haloalkyl, —OR^{G5}, —SR^{G5}, or —N(R^{G5})₂. In some embodiments, R¹ is 4-membered heterocyclyl independently substituted with 3 halo, C₁₋₆ alkyl, C₁₋₆ haloalkyl, —OR^{G5}, —SR^{G5}, or —N(R^{G5})₂.

In some embodiments, R¹ is 3-4 membered heterocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is halo.

In some embodiments, R¹ is 3-membered heterocyclyl substituted with at least one halo. In some embodiments, R¹ is 4-membered heterocyclyl substituted with at least one halo.

In some embodiments, R¹ is 3-4 membered heterocyclyl substituted with at least one of F, Cl, Br, or I. In some embodiments, R¹ is 3-4 membered heterocyclyl substituted with at least one of F, Cl, or Br.

In some embodiments, R¹ is 3-4 membered heterocyclyl substituted with at least one of F or C₁.

In some embodiments, R¹ is 3-membered heterocyclyl substituted with at least one of F, Cl, Br, or I. In some embodiments, R¹ is 3-membered heterocyclyl substituted with at least one of F, Cl, or Br. In some embodiments, R¹ is 3-membered heterocyclyl substituted with at least one of F or Cl.

In some embodiments, R¹ is 4-membered heterocyclyl substituted with at least one of F, Cl, Br, or I. In some embodiments, R¹ is 4-membered heterocyclyl substituted with at least one of F, Cl, or Br. In some embodiments, R¹ is 4-membered heterocyclyl substituted with at least one of F or Cl.

In some embodiments, R¹ is 3-4 membered heterocyclyl substituted with at least one F. In some embodiments, R¹ is 3-4 membered heterocyclyl substituted with at least one Cl. In some embodiments, R¹ is 3-4 membered heterocyclyl substituted with at least one Br. In some embodiments, R¹ is 3-4 membered heterocyclyl substituted with at least one I.

In some embodiments, R¹ is 3-membered heterocyclyl substituted with at least one F. In some embodiments, R¹ is 3-membered heterocyclyl substituted with at least one Cl. In some embodiments, R¹ is 3-membered heterocyclyl substituted with at least one Br. In some embodiments, R¹ is 3-membered heterocyclyl substituted with at least one I.

In some embodiments, R¹ is 4-membered heterocyclyl substituted with at least one F. In some embodiments, R¹ is 4-membered heterocyclyl substituted with at least one Cl. In some embodiments, R¹ is 4-membered heterocyclyl substituted with at least one Br. In some embodiments, R¹ is 4-membered heterocyclyl substituted with at least one I.

In some embodiments, R¹ is 3-4 membered heterocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is C₁₋₆ alkyl.

In some embodiments, R¹ is 3-membered heterocyclyl substituted with at least one C₁₋₆ alkyl. In some embodiments, R¹ is 4-membered heterocyclyl substituted with at least one C₁₋₆ alkyl.

In some embodiments, R¹ is 3-4 membered heterocyclyl substituted with at least one methyl. In some embodiments, R¹ is 3-4 membered heterocyclyl substituted with at least one ethyl. In some embodiments, R¹ is 3-4 membered heterocyclyl substituted with at least one propyl. In some embodiments, R¹ is 3-4 membered heterocyclyl substituted with at least one butyl. In some embodiments, R¹ is 3-4 membered heterocyclyl substituted with at least one pentyl. In some embodiments, R¹ is 3-4 membered heterocyclyl substituted with at least one hexyl. In some embodiments, R¹ is 3-4 membered heterocyclyl substituted with at least one isopropyl. In some embodiments, R¹ is 3-4 membered heterocyclyl substituted with at least one isobutyl. In some embodiments, R¹ is 3-4 membered heterocyclyl substituted with at least one isopentyl. In some embodiments, R¹ is 3-4 membered heterocyclyl substituted with at least one isohexyl. In some embodiments, R¹ is 3-4 membered heterocyclyl substituted with at least one secbutyl. In some embodiments, R¹ is 3-4 membered heterocyclyl substituted with at least one secpentyl. In some embodiments, R¹ is 3-4 membered heterocyclyl substituted with at least one sechexyl. In some embodiments, R¹ is 3-4 membered heterocyclyl substituted with at least one tertbutyl.

In some embodiments, R¹ is 3-membered heterocyclyl substituted with at least one methyl. In some embodiments, R¹ is 3-membered heterocyclyl substituted with at least one ethyl. In some embodiments, R¹ is 3-membered heterocyclyl substituted with at least one propyl. In some embodiments, R¹ is 3-membered heterocyclyl substituted with at least one butyl. In some embodiments, R¹ is 3-membered heterocyclyl substituted with at least one pentyl. In some embodiments, R¹ is 3-membered heterocyclyl substituted with at least one hexyl. In some embodiments, R¹ is 3-membered heterocyclyl substituted with at least one isopropyl. In some embodiments, R¹ is 3-membered heterocyclyl substituted with at least one isobutyl. In some embodiments, R¹ is 3-membered heterocyclyl substituted with at least one isopentyl. In some embodiments, R¹ is 3-membered heterocyclyl substituted with at least one isohexyl. In some embodiments, R¹ is 3-membered heterocyclyl substituted with at least one secbutyl. In some embodiments, R¹ is 3-membered heterocyclyl substituted with at least one secpentyl. In some embodiments, R¹ is 3-membered heterocyclyl substituted with at least one sechexyl. In some embodiments, R¹ is 3-membered heterocyclyl substituted with at least one tertbutyl.

In some embodiments, R¹ is 4-membered heterocyclyl substituted with at least one methyl. In some embodiments, R¹ is 4-membered heterocyclyl substituted with at least one ethyl. In some embodiments, R¹ is 4-membered heterocyclyl substituted with at least one propyl. In some embodiments, R¹ is 4-membered heterocyclyl substituted with at least one butyl. In some embodiments, R¹ is 4-membered heterocyclyl substituted with at least one pentyl. In some embodiments, R¹ is 4-membered heterocyclyl substituted with at least one hexyl. In some embodiments, R¹ is 4-membered heterocyclyl substituted with at least one isopropyl. In some embodiments, R¹ is 4-membered heterocyclyl substituted with at least one isobutyl. In some embodiments, R¹ is 4-membered heterocyclyl substituted with at least one isopentyl. In some embodiments, R¹ is 4-membered heterocyclyl substituted with at least one isohexyl. In some embodiments, R¹ is 4-membered heterocyclyl substituted with at least one secbutyl. In some embodiments, R¹ is 4-membered heterocyclyl substituted with at least one secpentyl. In some embodiments, R¹ is 4-membered heterocyclyl substituted with at least one sechexyl. In some embodiments, R¹ is 4-membered heterocyclyl substituted with at least one tertbutyl.

In some embodiments, R¹ is 3-4 membered heterocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is C₁₋₆ haloalkyl.

In some embodiments, R¹ is 3-membered heterocyclyl substituted with at least one C₁₋₆ haloalkyl. In some embodiments, R¹ is 4-membered heterocyclyl substituted with at least one C₁₋₆ haloalkyl.

In some embodiments, R¹ is 3-4 membered heterocyclyl substituted with at least one halomethyl. In some embodiments, R¹ is 3-4 membered heterocyclyl substituted with at least one haloethyl. In some embodiments, R¹ is 3-4 membered heterocyclyl substituted with at least one halopropyl. In some embodiments, R¹ is 3-4 membered heterocyclyl substituted with at least one halobutyl. In some embodiments, R¹ is 3-4 membered heterocyclyl substituted with at least one halopentyl. In some embodiments, R¹ is 3-4 membered heterocyclyl substituted with at least one halohexyl.

In some embodiments, R¹ is 3-membered heterocyclyl substituted with at least one halomethyl. In some embodiments, R¹ is 3-membered heterocyclyl substituted with at least one haloethyl. In some embodiments, R¹ is 3-membered heterocyclyl substituted with at least one halopropyl. In some embodiments, R¹ is 3-membered heterocyclyl substituted with at least one halobutyl. In some embodiments, R¹ is 3-membered heterocyclyl substituted with at least one halopentyl. In some embodiments, R¹ is 3-membered heterocyclyl substituted with at least one halohexyl.

In some embodiments, R¹ is 4-membered heterocyclyl substituted with at least one halomethyl. In some embodiments, R¹ is 4-membered heterocyclyl substituted with at least one haloethyl. In some embodiments, R¹ is 4-membered heterocyclyl substituted with at least one halopropyl. In some embodiments, R¹ is 4-membered heterocyclyl substituted with at least one halobutyl. In some embodiments, R¹ is 4-membered heterocyclyl substituted with at least one halopentyl. In some embodiments, R¹ is 4-membered heterocyclyl substituted with at least one halohexyl.

In some embodiments, R¹ is 3-4 membered heterocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is —OR^{G5}.

In some embodiments, R¹ is 3-membered heterocyclyl substituted with at least one —OR^{G5}. In some embodiments, R¹ is 4-membered heterocyclyl substituted with at least one —OR^{G5}.

In some embodiments, R¹ is 3-4 membered heterocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is —SR^{G5}.

In some embodiments, R¹ is 3-membered heterocyclyl substituted with at least one —SR^{G5}. In some embodiments, R¹ is 4-membered heterocyclyl substituted with at least one —SR^{G5}.

In some embodiments, R¹ is 3-4 membered heterocyclyl independently substituted with 1, 2, or 3 halo, C₁₋₆ alkyl, C₁₋₆ haloalkyl, —OR^{G5}, —SR^{G5}, or —N(R^{G5})₂, provided at least one substituent is —N(R^{G5})₂.

In some embodiments, R¹ is 3-membered heterocyclyl substituted with at least one —N(R^{G5})₂. In some embodiments, R¹ is 4-membered heterocyclyl substituted with at least one —N(R^{G5})₂.

In some embodiments, R¹ and G₂, together with the atoms to which they are attached, are joined to form a 5-membered heteroaryl ring independently substituted with 0, 1, 2, or 3 R^{G7} selected from the group consisting of halo, C₁₋₆alkyl, C₁₋₆haloalkyl, —OR^{G5}, —SR^{G5}, and —N(R^{G5})₂.

In some embodiments, R¹ and G₂, together with the atoms to which they are attached, are joined to form an unsubstituted 5-membered heteroaryl ring.

In some embodiments, R¹ and G₂, together with the atoms to which they are attached, are joined to form a 5-membered heteroaryl ring substituted with 1 R^{G7} selected from the group consisting of halo, C₁₋₆alkyl, C₁₋₆haloalkyl, —OR^{G5}, —SR^{G5}, and —N(R^{G5})₂.

In some embodiments, R¹ and G₂, together with the atoms to which they are attached, are joined to form a 5-membered heteroaryl ring independently substituted with 2 R^{G7} selected from the group consisting of halo, C₁₋₆ alkyl, C₁₋₆ haloalkyl, —OR^{G5}, —SR^{G5}, and —N(R^{G5})₂.

In some embodiments, R¹ and G₂, together with the atoms to which they are attached, are joined to form a 5-membered heteroaryl ring independently substituted with 3 R^{G7} selected from the group consisting of halo, C₁₋₆ alkyl, C₁₋₆ haloalkyl, —OR^{G5}, —SR^{G5}, and —N(R^{G5})₂.

In some embodiments, R¹ and G₂, together with the atoms to which they are attached, are joined to form a 5-membered heteroaryl ring independently substituted with 0, 1, 2, or 3 R^{G7} selected from the group consisting of halo, C₁₋₆alkyl, C₁₋₆haloalkyl, —OR^{G5}, —SR^{G5}, and —N(R^{G5})₂, provided at least one substituent is halo.

In some embodiments, R¹ and G₂, together with the atoms to which they are attached, are joined to form a 5-membered heteroaryl ring substituted with at least one of F, Cl, Br, or I. In some embodiments, R¹ and G₂, together with the atoms to which they are attached, are joined to form a 5-membered heteroaryl ring substituted with at least one of F, Cl, or Br. In some embodiments, R¹ and G₂, together with the atoms to which they are attached, are joined to form a 5-membered heteroaryl ring substituted with at least one of F or Cl.

In some embodiments, R¹ and G₂, together with the atoms to which they are attached, are joined to form a 5-membered heteroaryl ring substituted with at least one F. In some embodiments, R¹ and G₂, together with the atoms to which they are attached, are joined to form a 5-membered heteroaryl ring substituted with at least one Cl. In some embodiments, R¹ and G₂, together with the atoms to which they are attached, are joined to form a 5-membered heteroaryl ring substituted with at least one Br. In some embodiments, R¹ and G₂, together with the atoms to which they are attached, are joined to form a 5-membered heteroaryl ring substituted with at least one I.

In some embodiments, R¹ and G₂, together with the atoms to which they are attached, are joined to form a 5-membered heteroaryl ring independently substituted with 0, 1, 2, or 3 R^{G7} selected from the group consisting of halo, C₁₋₆alkyl, C₁₋₆haloalkyl, —OR^{G5}, —SR^{G5}, and —N(R^{G5})₂, provided at least one substituent is C₁₋₆ alkyl.

In some embodiments, R¹ and G₂, together with the atoms to which they are attached, are joined to form a 5-membered heteroaryl ring substituted with at least one methyl. In some embodiments, R¹ and G₂, together with the atoms to which they are attached, are joined to form a 5-membered heteroaryl ring substituted with at least one ethyl. In some embodiments, R¹ and G₂, together with the atoms to which they are attached, are joined to form a 5-membered heteroaryl ring substituted with at least one propyl. In some embodiments, R¹ and G₂, together with the atoms to which they are attached, are joined to form a 5-membered heteroaryl ring substituted with at least one butyl. In some embodiments, R¹ and G₂, together with the atoms to which they are attached, are joined to form a 5-membered heteroaryl ring substituted with at least one pentyl. In some embodiments, R¹ and G₂, together with the atoms to which they are attached, are joined to form a 5-membered heteroaryl ring substituted with at least one hexyl. In some embodiments, R¹ and G₂, together with the atoms to which they are attached, are joined to form a 5-membered heteroaryl ring substituted with at least one isopropyl. In some embodiments, R¹ and G₂, together with the atoms to which they are attached, are joined to form a 5-membered heteroaryl ring substituted with at least one isobutyl. In some embodiments, R¹ and G₂, together with the atoms to which they are attached, are joined to form a 5-membered heteroaryl ring substituted with at least one isopentyl. In some embodiments, R¹ and G₂, together with the atoms to which they are attached, are joined to form a 5-membered heteroaryl ring substituted with at least one isohexyl. In some embodiments, R¹ and G₂, together with the atoms to which they are attached, are joined to form a 5-membered heteroaryl ring substituted with at least one secbutyl. In some embodiments, R¹ and G₂, together with the atoms to which they are attached, are joined to form a 5-membered heteroaryl ring substituted with at least one secpentyl.

In some embodiments, R¹ and G₂, together with the atoms to which they are attached, are joined to form a 5-membered heteroaryl ring substituted with at least one sechexyl. In some embodiments, R¹ and G₂, together with the atoms to which they are attached, are joined to form a 5-membered heteroaryl ring substituted with at least one tertbutyl.

In some embodiments, R¹ and G₂, together with the atoms to which they are attached, are joined to form a 5-membered heteroaryl ring independently substituted with 0, 1, 2, or 3 R^{G7} selected from the group consisting of halo, C₁₋₆alkyl, C₁₋₆haloalkyl, —OR^{G5}, —SR^{G5}, and —N(R^{G5})₂, provided at least one substituent is C₁₋₆ haloalkyl.

In some embodiments, R¹ and G₂, together with the atoms to which they are attached, are joined to form a 5-membered heteroaryl ring substituted with at least one halomethyl. In some embodiments, R¹ and G₂, together with the atoms to which they are attached, are joined to form a 5-membered heteroaryl ring substituted with at least one haloethyl. In some embodiments, R¹ and G₂, together with the atoms to which they are attached, are joined to form a 5-membered heteroaryl ring substituted with at least one halopropyl. In some embodiments, R¹ and G₂, together with the atoms to which they are attached, are joined to form a 5-membered heteroaryl ring substituted with at least one halobutyl. In some embodiments, R¹ and G₂, together with the atoms to which they are attached, are joined to form a 5-membered heteroaryl ring substituted with at least one halopentyl. In some embodiments, R¹ and G₂, together with the atoms to which they are attached, are joined to form a 5-membered heteroaryl ring substituted with at least one halohexyl.

In some embodiments, R¹ and G₂, together with the atoms to which they are attached, are joined to form a 5-membered heteroaryl ring independently substituted with 0, 1, 2, or 3 R^{G7} groups selected from the group consisting of halo, C₁₋₆ alkyl, C₁₋₆ haloalkyl, —OR^{G5}, —SR^{G5}, and —N(R^{G5})₂, provided at least one substituent is —OR^{G5}.

In some embodiments, R¹ and G₂, together with the atoms to which they are attached, are joined to form a 5-membered heteroaryl ring independently substituted with 0, 1, 2, or 3 R^{G7} groups selected from the group consisting of halo, C₁₋₆ alkyl, C₁₋₆ haloalkyl, —OR^{G5}, —SR^{G5}, and —N(R^{G5})₂, provided at least one substituent is —SR^{G5}.

In some embodiments, R¹ and G₂, together with the atoms to which they are attached, are joined to form a 5-membered heteroaryl ring independently substituted with 0, 1, 2, or 3 R^{G7} groups selected from the group consisting of halo, C₁₋₆ alkyl, C₁₋₆ haloalkyl, —OR^{G5}, —SR^{G5}, and —N(R^{G5})₂, provided at least one substituent is —N(R^{G5})₂.

As generally defined herein, each instance of R^{G7} is independently halo, C₁₋₆ alkyl, C₁₋₆ haloalkyl, —OR^{G5}, —SR^{G5}, or —N(R^{G5})₂.

In some embodiments, R^{G7} is halo.

In some embodiments, R^{G7} is F, Cl, Br, or I. In some embodiments, R^{G7} is F, Cl, or Br. In some embodiments, R^{G7} is F or Cl.

In some embodiments, R^{G7} is F. In some embodiments, R^{G7} is Cl. In some embodiments, R^{G7} is Br. In some embodiments, R^{G7} is I.

In some embodiments, R^{G7} is C₁₋₆ alkyl.

In some embodiments, R^{G7} is methyl. In some embodiments, R^{G7} is ethyl. In some embodiments, R^{G7} is propyl. In some embodiments, R^{G7} is butyl. In some embodiments, R^{G7} is pentyl. In some embodiments, R^{G7} is hexyl. In some embodiments, R^{G7} is isopropyl. In some embodiments, R^{G7} is isobutyl. In some embodiments, R^{G7} is isopentyl. In some embodiments, R^{G7} is isohexyl. In some embodiments, R^{G7} is secbutyl. In some embodiments, R^{G7} is secpentyl. In some embodiments, R^{G7} is sechexyl. In some embodiments, R^{G7} is tertbutyl.

In some embodiments, R^{G7} is C₁₋₆ haloalkyl.

In some embodiments, R^{G7} is halomethyl. In some embodiments, R^{G7} is haloethyl. In some embodiments, R^{G7} is halopropyl. In some embodiments, R^{G7} is halobutyl. In some embodiments, R^{G7} is halopentyl. In some embodiments, R^{G7} is halohexyl.

In some embodiments, R^{G7} is —OR^{G5}.

In some embodiments, R^{G7} is —SR^{G5}.

In some embodiments, R^{G7} is —N(R^{G5})₂.

As generally defined herein, R^{G1}, R^{G2}, R^{G3}, and R^{G4} are each independently selected from the group consisting of hydrogen, halo, C₁₋₆ alkyl, C₁₋₆ haloalkyl, and —OR^{G6}.

In some embodiments, R^{G1} is selected from the group consisting of hydrogen, halo, C₁₋₆ alkyl, C₁₋₆ haloalkyl, and —OR^{G6}.

In some embodiments, R^{G1} is hydrogen.

In some embodiments, R^{G1} is halo.

In some embodiments, R^{G1} is F, Cl, Br, or I. In some embodiments, R^{G1} is F, Cl, or Br. In some embodiments, R^{G1} is F or Cl.

In some embodiments, R^{G1} is F. In some embodiments, R^{G1} is Cl. In some embodiments, R^{G1} is Br. In some embodiments, R^{G1} is I.

In some embodiments, R^{G1} is C₁₋₆ alkyl.

In some embodiments, R^{G1} is methyl. In some embodiments, R^{G1} is ethyl. In some embodiments, R^{G1} is propyl. In some embodiments, R^{G1} is butyl. In some embodiments, R^{G1} is pentyl. In some embodiments, R^{G1} is hexyl. In some embodiments, R^{G1} is isopropyl. In some embodiments, R^{G1} is isobutyl. In some embodiments, R^{G1} is isopentyl. In some embodiments, R^{G1} is isohexyl. In some embodiments, R^{G1} is secbutyl. In some embodiments, R^{G1} is secpentyl. In some embodiments, R^{G1} is sechexyl. In some embodiments, R^{G1} is tertbutyl.

In some embodiments, R^{G1} is C₁₋₆haloalkyl.

In some embodiments, R^{G1} is halomethyl. In some embodiments, R^{G1} is haloethyl. In some embodiments, R^{G1} is halopropyl. In some embodiments, R^{G1} is halobutyl. In some embodiments, R^{G1} is halopentyl. In some embodiments, R^{G1} is halohexyl.

In some embodiments, R^{G1} is —OR^{G6}.

In some embodiments, R^{G2} is selected from the group consisting of hydrogen, halo, C₁₋₆ alkyl, C₁₋₆ haloalkyl, and —OR^{G6}.

In some embodiments, R^{G2} is hydrogen.

In some embodiments, R^{G2} is halo.

In some embodiments, R^{G2} is F, Cl, Br, or I. In some embodiments, R^{G2} is F, Cl, or Br. In some embodiments, R^{G2} is F or Cl.

In some embodiments, R^{G2} is F. In some embodiments, R^{G2} is Cl. In some embodiments, R^{G2} is Br. In some embodiments, R^{G2} is I.

In some embodiments, R^{G2} is C₁₋₆ alkyl.

In some embodiments, R^{G2} is methyl. In some embodiments, R^{G2} is ethyl. In some embodiments, R^{G2} is propyl. In some embodiments, R^{G2} is butyl. In some embodiments, R^{G2} is pentyl. In some embodiments, R^{G2} is hexyl. In some embodiments, R^{G2} is isopropyl. In some embodiments, R^{G2} is isobutyl. In some embodiments, R^{G2} is isopentyl. In some embodiments, R^{G2} is isohexyl. In some embodiments, R^{G2} is secbutyl. In some embodiments, R^{G2} is secpentyl. In some embodiments, R^{G2} is sechexyl. In some embodiments, R^{G2} is tertbutyl.

In some embodiments, R^{G2} is C₁₋₆haloalkyl.

In some embodiments, R^{G2} is halomethyl. In some embodiments, R^{G2} is haloethyl. In some embodiments, R^{G2} is halopropyl. In some embodiments, R^{G2} is halobutyl. In some embodiments, R^{G2} is halopentyl. In some embodiments, R^{G2} is halohexyl.

In some embodiments, R^{G2} is —OR^{G6}.

In some embodiments, R^{G3} is selected from the group consisting of hydrogen, halo, C₁₋₆ alkyl, C₁₋₆ haloalkyl, and —OR^{G6}.

In some embodiments, R^{G3} is hydrogen.

In some embodiments, R^{G3} is halo.

In some embodiments, R^{G3} is F, Cl, Br, or I. In some embodiments, R^{G3} is F, Cl, or Br. In some embodiments, R^{G3} is F or Cl.

In some embodiments, R^{G3} is F. In some embodiments, R^{G3} is Cl. In some embodiments, R^{G3} is Br. In some embodiments, R^{G3} is I.

In some embodiments, R^{G3} is C₁₋₆alkyl.

In some embodiments, R^{G3} is methyl. In some embodiments, R^{G3} is ethyl. In some embodiments, R^{G3} is propyl. In some embodiments, R^{G3} is butyl. In some embodiments, R^{G3} is pentyl. In some embodiments, R^{G3} is hexyl. In some embodiments, R^{G3} is isopropyl. In some embodiments, R^{G3} is isobutyl. In some embodiments, R^{G3} is isopentyl. In some embodiments, R^{G3} is isohexyl. In some embodiments, R^{G3} is secbutyl. In some embodiments, R^{G3} is secpentyl. In some embodiments, R^{G3} is sechexyl. In some embodiments, R^{G3} is tertbutyl.

In some embodiments, R^{G3} is C₁₋₆ haloalkyl.

In some embodiments, R^{G3} is halomethyl. In some embodiments, R^{G3} is haloethyl. In some embodiments, R^{G3} is halopropyl. In some embodiments, R^{G3} is halobutyl. In some embodiments, R^{G3} is halopentyl. In some embodiments, R^{G3} is halohexyl.

In some embodiments, R^{G3} is —OR^{G6}.

In some embodiments, R^{G4} is selected from the group consisting of hydrogen, halo, C₁₋₆ alkyl, C₁₋₆ haloalkyl, and —OR^{G6}.

In some embodiments, R^{G4} is hydrogen.

In some embodiments, R^{G4} is halo.

In some embodiments, R^{G4} is F, Cl, Br, or I. In some embodiments, R^{G4} is F, Cl, or Br. In some embodiments, R^{G4} is F or Cl.

In some embodiments, R^{G4} is F. In some embodiments, R^{G4} is Cl. In some embodiments, R^{G4} is Br. In some embodiments, R^{G4} is I.

In some embodiments, R^{G4} is C₁₋₆ alkyl.

In some embodiments, R^{G4} is methyl. In some embodiments, R^{G4} is ethyl. In some embodiments, R^{G4} is propyl. In some embodiments, R^{G4} is butyl. In some embodiments, R^{G4} is pentyl. In some embodiments, R^{G4} is hexyl. In some embodiments, R^{G4} is isopropyl. In some embodiments, R^{G4} is isobutyl. In some embodiments, R^{G4} is isopentyl. In some embodiments, R^{G4} is isohexyl. In some embodiments, R^{G4} is secbutyl. In some embodiments, R^{G4} is secpentyl. In some embodiments, R^{G4} is sechexyl. In some embodiments, R^{G4} is tertbutyl.

In some embodiments, R^{G4} is C₁₋₆ haloalkyl.

In some embodiments, R^{G4} is halomethyl. In some embodiments, R^{G4} is haloethyl. In some embodiments, R^{G4} is halopropyl. In some embodiments, R^{G4} is halobutyl. In some embodiments, R^{G4} is halopentyl. In some embodiments, R^{G4} is halohexyl.

In some embodiments, R^{G4} is —OR^{G6}.

As generally defined herein, R^{G5} and R^{G6} are each independently hydrogen, C₁₋₆ alkyl, or C₁₋₆ haloalkyl.

In some embodiments, R^{G5} is hydrogen.

In some embodiments, R^{G5} is C₁₋₆ alkyl.

In some embodiments, R^{G5} is methyl. In some embodiments, R^{G5} is ethyl. In some embodiments, R^{G5} is propyl. In some embodiments, R^{G5} is butyl. In some embodiments, R^{G5} is pentyl. In some embodiments, R^{G5} is hexyl. In some embodiments, R^{G5} is isopropyl. In some embodiments, R^{G5} is isobutyl. In some embodiments, R^{G5} is isopentyl. In some embodiments, R^{G5} is isohexyl. In some embodiments, R^{G5} is secbutyl. In some embodiments, R^{G5} is secpentyl. In some embodiments, R^{G5} is sechexyl. In some embodiments, R^{G5} is tertbutyl.

In some embodiments, R^{G5} is C₁₋₆ haloalkyl.

In some embodiments, R^{G5} is halomethyl. In some embodiments, R^{G5} is haloethyl. In some embodiments, R^{G5} is halopropyl. In some embodiments, R^{G5} is halobutyl. In some embodiments, R^{G5} is halopentyl. In some embodiments, R^{G5} is halohexyl.

In some embodiments, R^{G6} is hydrogen.

In some embodiments, R^{G6} is C₁₋₆ alkyl.

In some embodiments, R^{G6} is methyl. In some embodiments, R^{G6} is ethyl. In some embodiments, R^{G6} is propyl. In some embodiments, R^{G6} is butyl. In some embodiments, R^{G6} is pentyl. In some embodiments, R^{G6} is hexyl. In some embodiments, R^{G6} is isopropyl. In some embodiments, R^{G6} is isobutyl. In some embodiments, R^{G6} is isopentyl. In some embodiments, R^{G6} is isohexyl. In some embodiments, R^{G6} is secbutyl. In some embodiments, R^{G6} is secpentyl. In some embodiments, R^{G6} is sechexyl. In some embodiments, R^{G6} is tertbutyl.

In some embodiments, R^{G6} is C₁₋₆haloalkyl.

In some embodiments, R^{G6} is halomethyl. In some embodiments, R^{G6} is haloethyl. In some embodiments, R^{G6} is halopropyl. In some embodiments, R^{G6} is halobutyl. In some embodiments, R^{G6} is halopentyl. In some embodiments, R^{G6} is halohexyl.

In some embodiments, Ring A of formula:

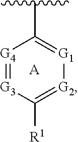

R is a group of formula:

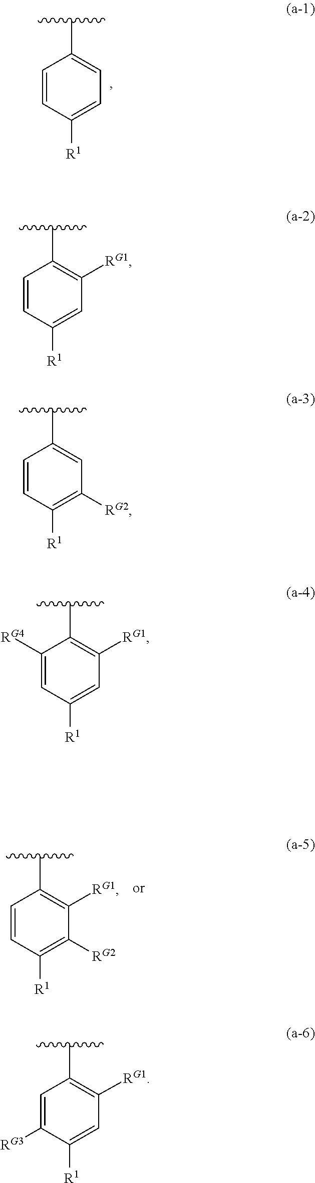

In some embodiments, Ring A is of formula (a-2), (a-3), (a-4), (a-5), or (a-6), wherein R^{G1}, R^{G2}, R^{G3}, and R^{G4} are each independently selected from the group consisting of halo, C₁₋₆ alkyl, C₁₋₆ haloalkyl, and —OR^{G6}.

In some embodiments, Ring A is of formula (a-2), (a-4), (a-5), or (a-6), wherein R^{G1} is —OR^{G6}.

In some embodiments, Ring A is of formula:

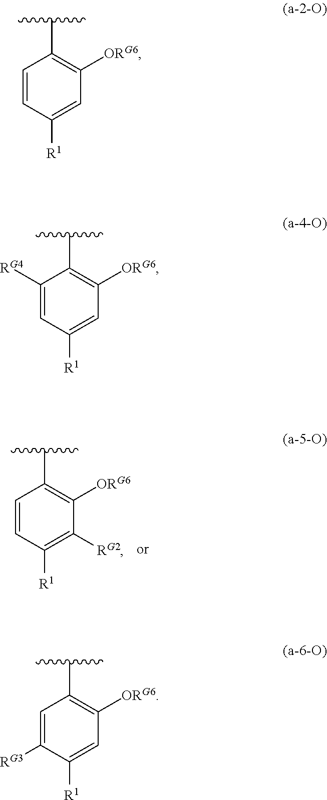

In some embodiments, Ring A is of formula (a-2), (a-4), (a-5), or (a-6), wherein R^{G1} is fluoro.

In some embodiments, Ring A is of formula:

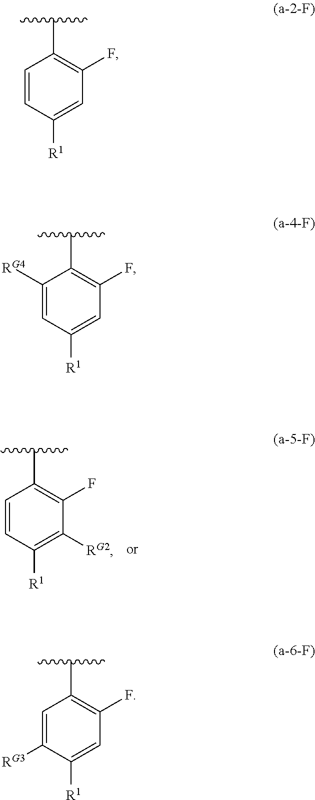

In some embodiments, Ring A of formula:

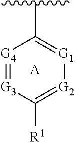

R is a group of formula:

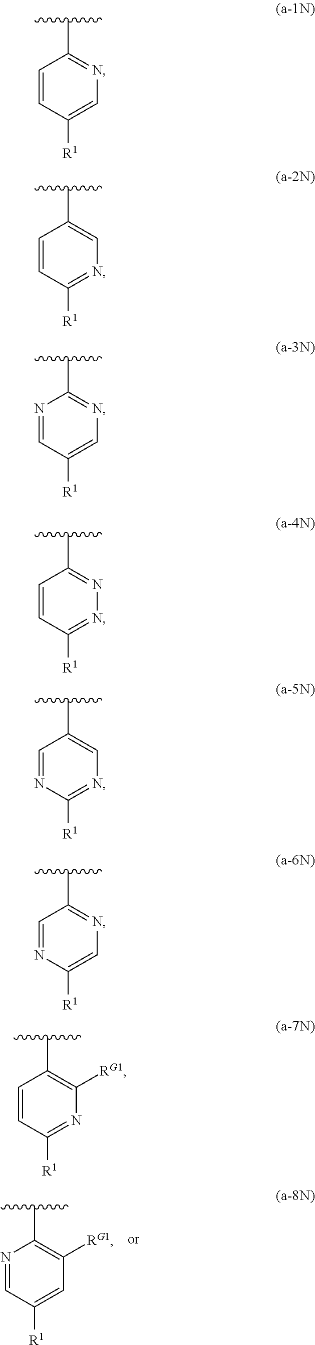

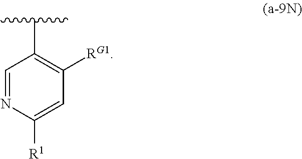

In some embodiments, Ring A is of formula (a-1N), (a-2N), (a-3N), (a-4N), (a-5N), (a-6N), (a-7N), (a-8N), or (a-9N), wherein R^{G1} is halo, C₁₋₆alkyl, C₁₋₆haloalkyl, or —OR^{G6}.

In some embodiments, Ring A is of formula (a-7N), (a-8N), or (a-9N), wherein R^{G1} is —OR^{G6}.

In some embodiments, Ring A is of formula:

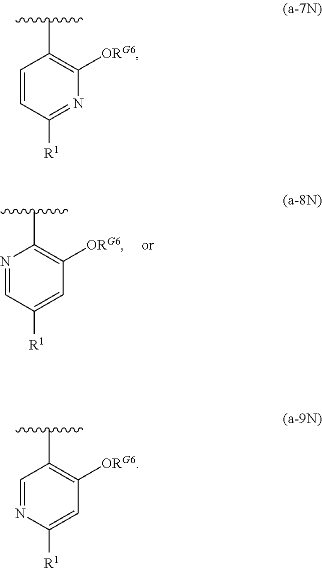

In some embodiments, Ring A is of formula:

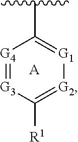

and R¹ and G₂, together with the atoms to which they are attached, are joined to form a 5-membered heteroaryl ring, wherein the Ring A, R¹, and G² provide a group of formula:

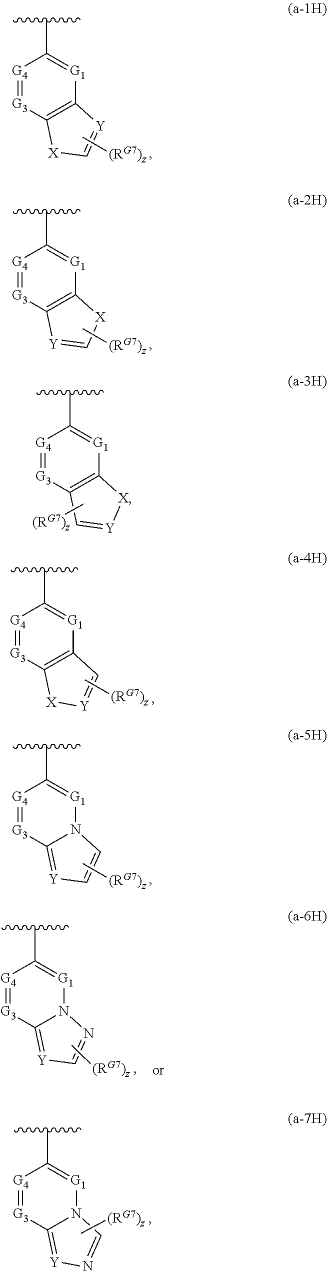

wherein:
X is O, S, NH, or NR^{G7};
Y is N, CH, or CR^{G7}; ad
z is 0 or 1;
provided if R^{G7} is a group attached to a nitrogen (N) atom, then R^{G7} is C₁₋₆ alkyl or C₁₋₆ haloalkyl.

In some embodiments, X is O or S.

In some embodiments, X is O. In some embodiments, X is S.

In some embodiments, X is NH or NR^{G7}.

In some embodiments, X is NH. In some embodiments, X is NR^{G7}.

In some embodiments, Y is N.

In some embodiments, Y is CH or CR^{G7}.

In some embodiments, Y is CH. In some embodiments, Y is CR^{G7}.

In some embodiments, z is 0.

In some embodiments, z is 1.

In some embodiments, if R^{G7} is a group attached to a nitrogen (N) atom, then R^{G7} is C₁₋₆ alkyl.

In some embodiments, if R^{G7} is a group attached to a nitrogen (N) atom, then R^{G7} is C₁₋₆ haloalkyl.

In some embodiments, Ring A, when R¹ and G₂, together with the atoms to which they are attached, are joined to form a 5-membered heteroaryl ring, wherein the Ring A, R¹, and G² provide a group of formula:

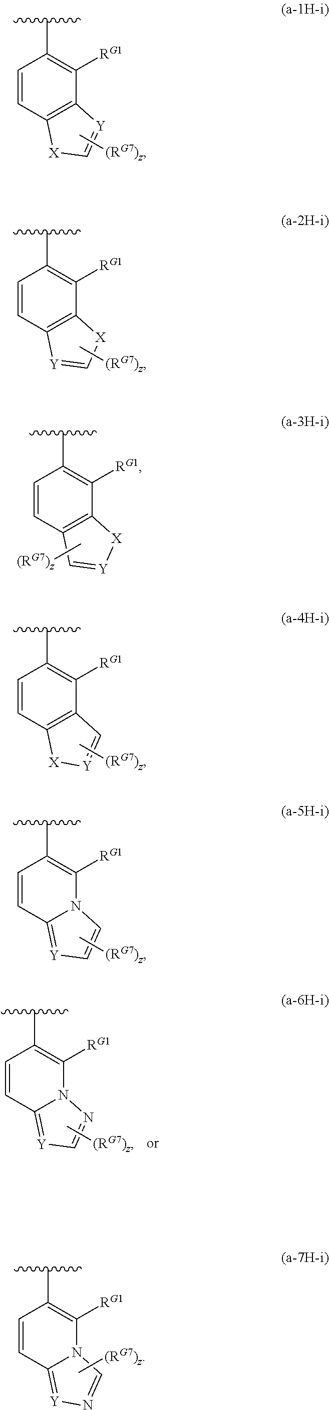

In some embodiments, Ring A is a group of formula:

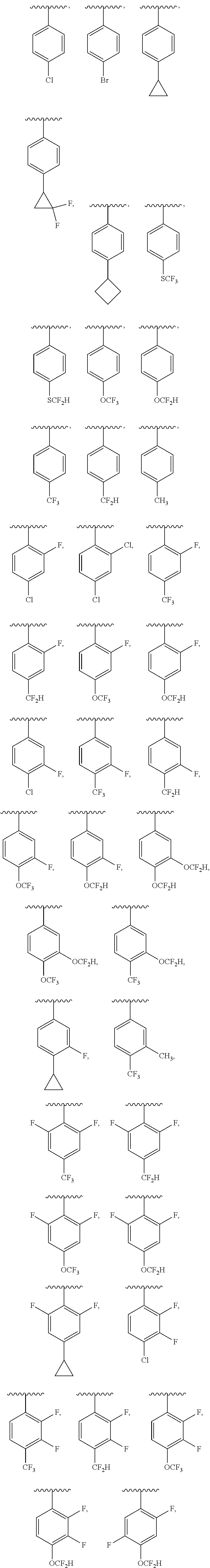

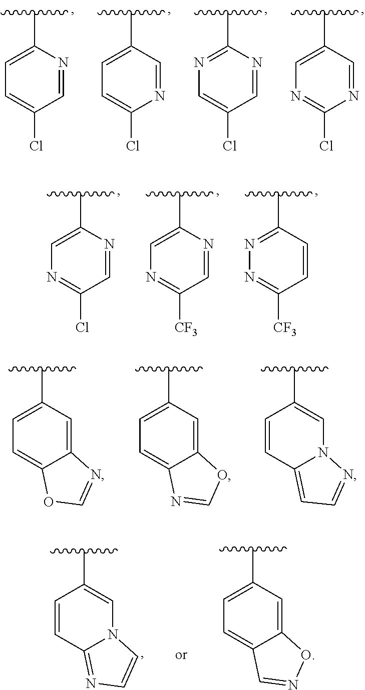

In some embodiments, Ring A is a group of formula:

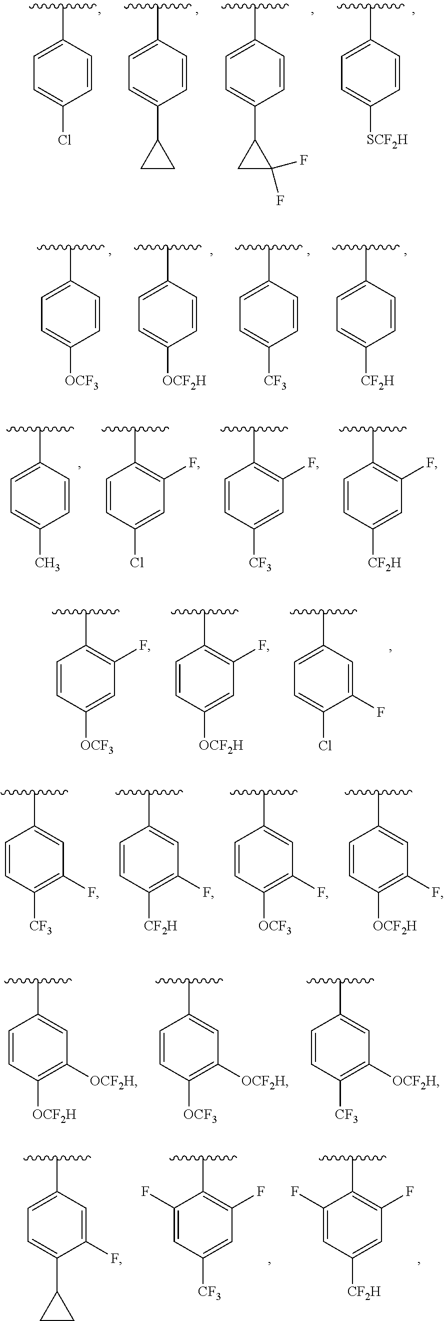

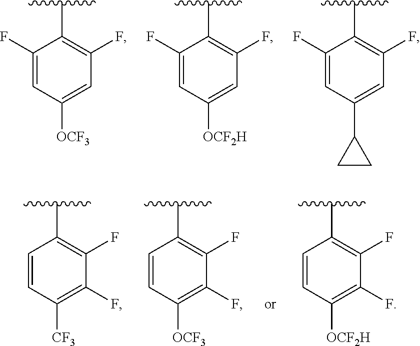

In some embodiments, Ring A is a group of formula:

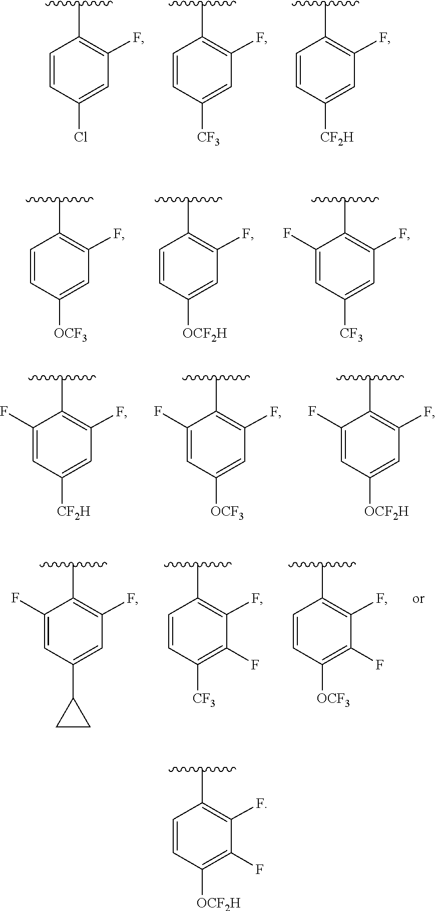

In some embodiments, Ring A is a group of formula:

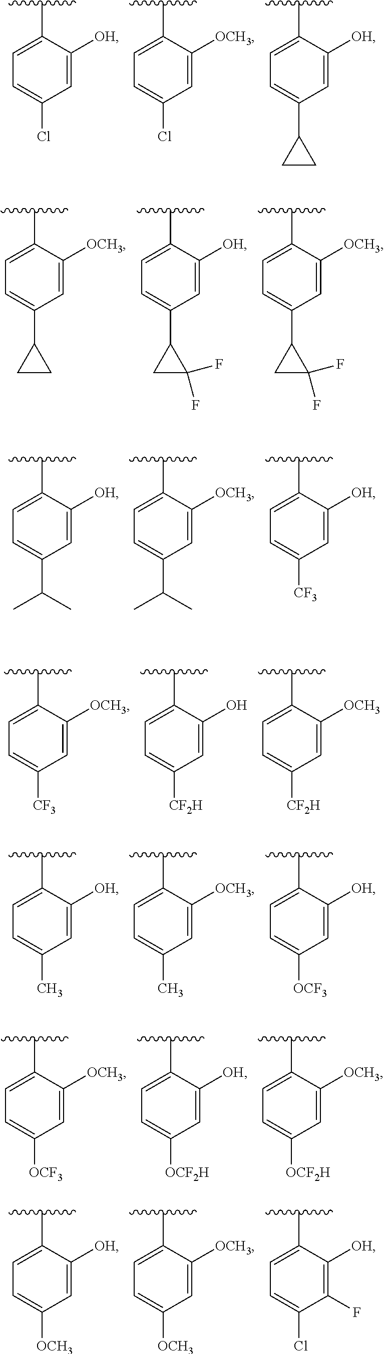

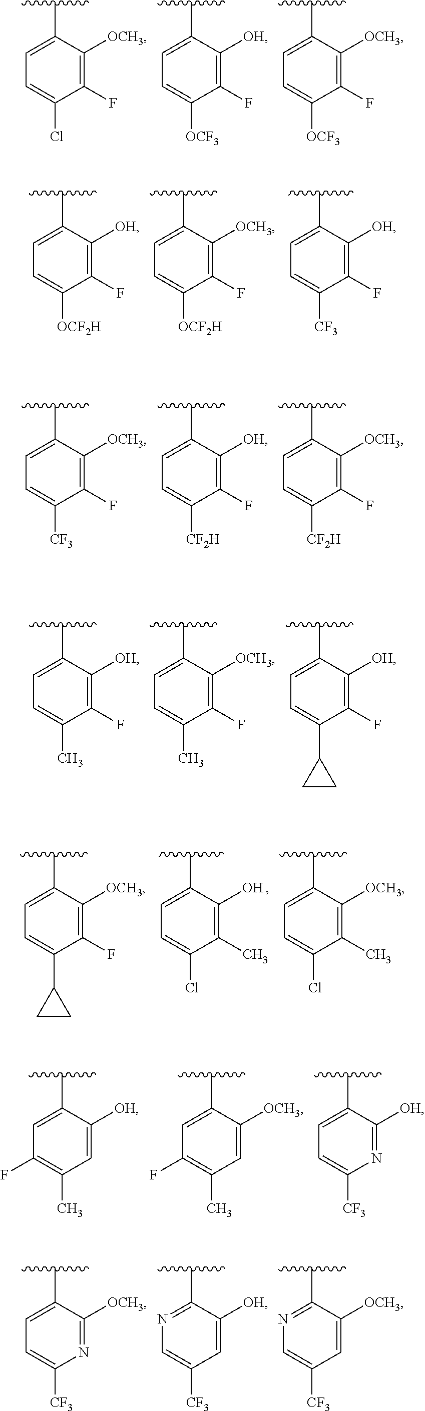

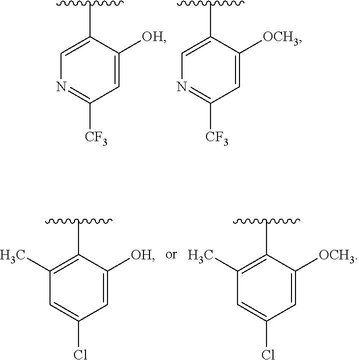

In some embodiments, Ring A is a group of formula:

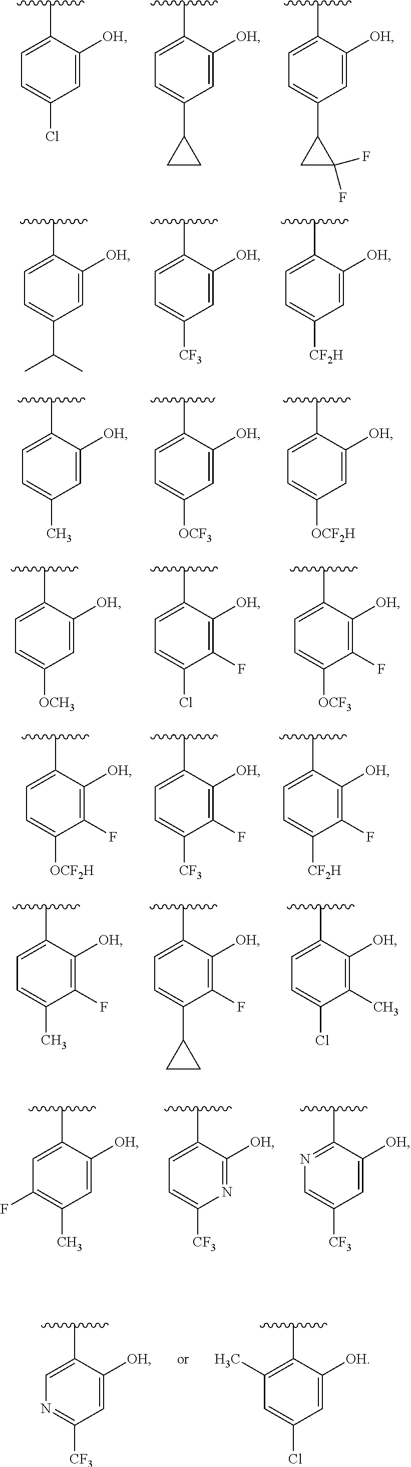

In some embodiments, Ring A is a group of formula:

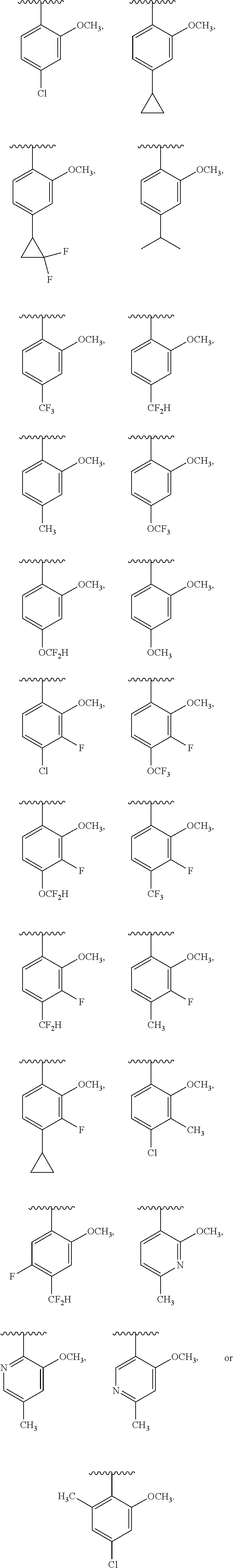

In some embodiments, Ring A is a group of formula:

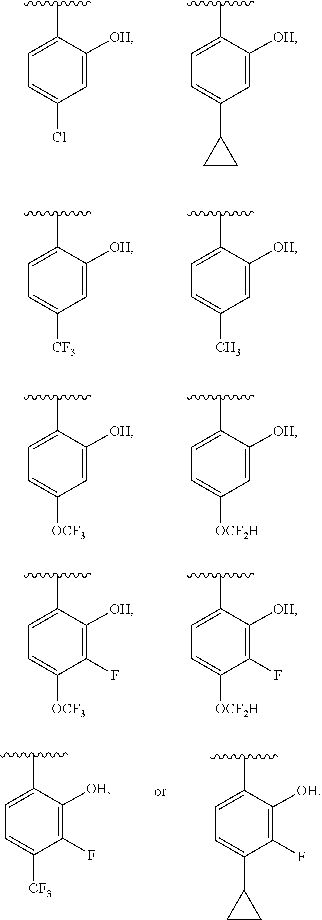

(b) Ring B, n, p, m, R³, R^{2a}, and R^{2b}

As generally described herein, n is 0 or 1.

In some embodiments, n is 0 or 1.

In some embodiments, n is 0. In some embodiments, n is 1.

As generally described herein, p is 1 or 2.

In some embodiments, p is 1. In some embodiments, p is 2.

As generally described herein, m is 0, 1, 2, or 3.

In some embodiments, m is 1, 2, or 3. In some embodiments, m is 0, 1, or 2. In some embodiments, m is 1 or 2. In some embodiments, m is 2 or 3. In some embodiments, m is 1 or 3.

In some embodiments, m is 0. In some embodiments, m is 1. In some embodiments, m is 2. In some embodiments, m is 3.

As generally described herein, each instance of R^{2a} and R^{2b} is independently hydrogen, halo, C₁₋₆ alkyl, C₁₋₆ haloalkyl, C₃-C₄ carbocyclyl, or 3-4 membered heterocyclyl, wherein the carbocyclyl or heterocyclyl are each independently substituted with 0, 1, 2, or 3 halo, or R^{2a} and R^{2b} are joined to form a C₃ carbocyclyl independently substituted with 0, 1, 2, or 3 halo.

In some embodiments, R^{2a} is independently hydrogen, halo, C₁₋₆ alkyl, C₁₋₆ haloalkyl, C₃-C₄ carbocyclyl, or 3-4 membered heterocyclyl.

In some embodiments, R^{2a} is independently hydrogen.

In some embodiments, R^{2a} is independently halo.

In some embodiments, R^{2a} is independently F, Cl, Br, or I. In some embodiments, R^{2a} is independently F, Cl, or Br. In some embodiments, R^{2a} is independently F or Cl.

In some embodiments, R^{2a} is independently F. In some embodiments, R^{2a} is independently Cl. In some embodiments, R^{2a} is independently Br. In some embodiments, R^{2a} is independently I.

In some embodiments, R^{2a} is independently C₁₋₆ alkyl.

In some embodiments, R^{2′} is independently methyl. In some embodiments, R^{2′} is independently ethyl. In some embodiments, R^{2a} is independently propyl. In some embodiments, R^{2a} is independently butyl. In some embodiments, R^{2a} is independently pentyl. In some embodiments, R^{2a} is independently hexyl. In some embodiments, R^{2a} is independently isopropyl. In some embodiments, R^{2a} is independently isobutyl. In some embodiments, R^{2a} is independently isopentyl. In some embodiments, R^{2a} is independently isohexyl. In some embodiments, R^{2a} is independently secbutyl. In some embodiments, R^{2a} is independently secpentyl. In some embodiments, R^{2a} is independently sechexyl. In some embodiments, R^{2a} is independently tertbutyl.

In some embodiments, R^{2a} is independently C₁₋₆haloalkyl.

In some embodiments, R^{2a} is independently halomethyl. In some embodiments, R^{2a} is independently haloethyl. In some embodiments, R^{2a} is independently halopropyl. In some embodiments, R^{2a} is independently halobutyl. In some embodiments, R^{2a} is independently halopentyl. In some embodiments, R^{2a} is independently halohexyl.

In some embodiments, R^{2a} is independently C₃-C₄ carbocyclyl.

In some embodiments, R^{2a} is independently C₃ carbocyclyl. In some embodiments, R^{2a} is independently C₄ carbocyclyl.

In some embodiments, R^{2a} is independently 3-4 membered heterocyclyl.

In some embodiments, R^{2a} is independently 3-membered heterocyclyl. In some embodiments, R^{2a} is independently 4-membered heterocyclyl.

In some embodiments, each instance of R^{2b} is independently hydrogen, halo, C₁₋₆ alkyl, C₁₋₆ haloalkyl, C₃-C₄ carbocyclyl, or 3-4 membered heterocyclyl.

In some embodiments, R^{2b} is independently hydrogen.

In some embodiments, R^{2b} is independently halo.

In some embodiments, R^{2b} is independently F, Cl, Br, or I. In some embodiments, R^{2b} is independently F, Cl, or Br. In some embodiments, R^{2b} is independently F or Cl.

In some embodiments, R^{2b} is independently F. In some embodiments, R^{2b} is independently Cl. In some embodiments, R^{2b} is independently Br. In some embodiments, R^{2b} is independently I.

In some embodiments, R^{2b} is independently C₁₋₆ alkyl.

In some embodiments, R^{2b} is independently methyl. In some embodiments, R^{2b} is independently ethyl. In some embodiments, R^{2b} is independently propyl. In some embodiments, R^{2b} is independently butyl. In some embodiments, R^{2b} is independently pentyl. In some embodiments, R^{2b} is independently hexyl. In some embodiments, R^{2b} is independently isopropyl. In some embodiments, R^{2b} is independently isobutyl. In some embodiments, R^{2b} is independently isopentyl. In some embodiments, R^{2b} is independently isohexyl. In some embodiments, R^{2b} is independently secbutyl. In some embodiments, R^{2b} is independently secpentyl. In some embodiments, R^{2b} is independently sechexyl. In some embodiments, R^{2b} is independently tertbutyl.

In some embodiments, R^{2b} is independently C₁₋₆ haloalkyl.

In some embodiments, R^{2b} is independently halomethyl. In some embodiments, R^{2b} is independently haloethyl. In some embodiments, R^{2b} is independently halopropyl. In some embodiments, R^{2b} is independently halobutyl. In some embodiments, R^{2b} is independently halopentyl. In some embodiments, R^{2b} is independently halohexyl.

In some embodiments, R^{2b} is independently C₃-C₄ carbocyclyl.

In some embodiments, R^{2b} is independently C₃ carbocyclyl. In some embodiments, R^{2b} is independently C₄ carbocyclyl.

In some embodiments, R^{2b} is independently 3-4 membered heterocyclyl.

In some embodiments, R^{2b} is independently 3-membered heterocyclyl. In some embodiments, R^{2b} is independently 4-membered heterocyclyl.

In some embodiments, R^{2a} and R^{2b} are the same. In some embodiments, R^{2a} and R^{2b} are different.

In some embodiments, R^{2a} and R^{2b} are joined to form a C₃ carbocyclyl independently substituted with 0, 1, 2, or 3 halo.

As generally described herein, each instance of R³ is independently halo, C₁₋₆ alkyl or C₁₋₆ haloalkyl, or two R³ groups are joined to form a C₁₋₃ alkylene bridging group or C₁₋₃ haloalkylene bridging group.

In some embodiments, R³ is halo.

In some embodiments, R³ is F, Cl, Br, or I. In some embodiments, R³ is F, Cl, or Br. In some embodiments, R³ is F or Cl.

In some embodiments, R³ is F. In some embodiments, R³ is Cl. In some embodiments, R³ is Br. In some embodiments, R³ is I.

In some embodiments, R³ is C₁₋₆alkyl.

In some embodiments, R³ is methyl. In some embodiments, R³ is ethyl. In some embodiments, R³ is propyl. In some embodiments, R³ is butyl. In some embodiments, R³ is pentyl. In some embodiments, R³ is hexyl. In some embodiments, R³ is isopropyl. In some embodiments, R³ is isobutyl. In some embodiments, R³ is isopentyl. In some embodiments, R³ is isohexyl. In some embodiments, R³ is secbutyl. In some embodiments, R³ is secpentyl. In some embodiments, R³ is sechexyl. In some embodiments, R³ is tertbutyl.

In some embodiments, R³ is C₁₋₆haloalkyl.

In some embodiments, R³ is halomethyl. In some embodiments, R³ is haloethyl. In some embodiments, R³ is halopropyl. In some embodiments, R³ is halobutyl. In some embodiments, R³ is halopentyl. In some embodiments, R³ is halohexyl.

As generally described herein, two R³ groups are joined to form a C₁₋₃ alkylene bridging group or a C₁₋₃ haloalkylene bridging group.

In some embodiments, two R³ groups are joined to form a bridging group are defined as L.

In some embodiments, two R³ groups are joined to form a C₁₋₃ alkylene bridging group.

In some embodiments, two R³ groups are joined to form a methylene bridging group. In some embodiments, two R³ groups are joined to form an ethylene bridging group. In some embodiments, two R³ groups are joined to form a propylene bridging group.

In some embodiments, two R³ groups are joined to form a C₁₋₃ haloalkylene bridging group.

In some embodiments, two R³ groups are joined to form a halomethylene bridging group. In some embodiments, two R³ groups are joined to form a haloethylene bridging group. In some embodiments, two R³ groups are joined to form a halopropylene bridging group.

In some embodiments, Ring B of formula:

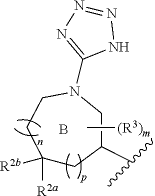

is a group of formula:

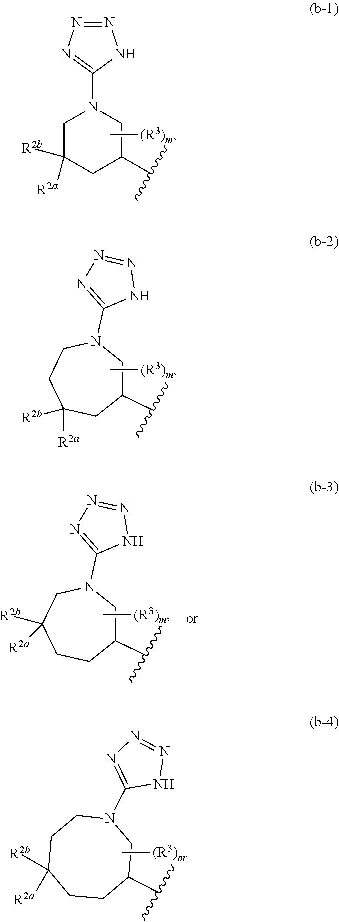

In some embodiments, Ring B is a group of formula:

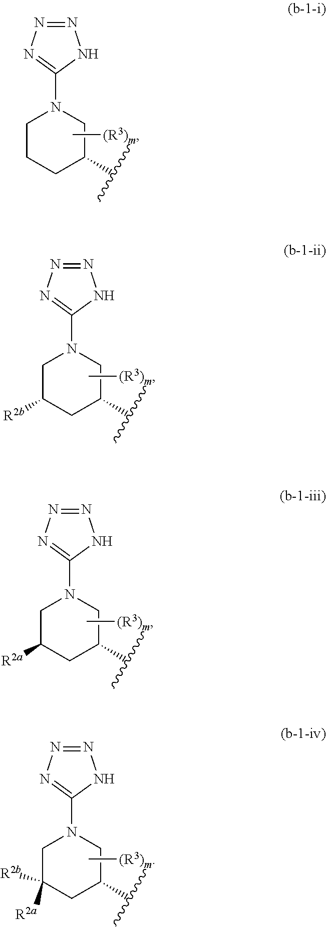

In some embodiments, Ring B is a group of formula (b-1-i), (b-1-ii), (b-1-iii), or (b-1-iv), wherein each instance of R^{2a} and R^{2b} is independently halo, C₁₋₆ alkyl, C₁₋₆ haloalkyl, C₃-C₄ carbocyclyl, or 3-4 membered heterocyclyl.

In some embodiments, Ring B is of formula (b-1), (b-2), (b-3), or (b-4), wherein two R³ groups, together with the atoms to which they are attached, are joined to form a C₁₋₃ alkylene bridging group or C₁₋₃ haloalkylene bridging group.

In some embodiments, Ring B is a group of formula:

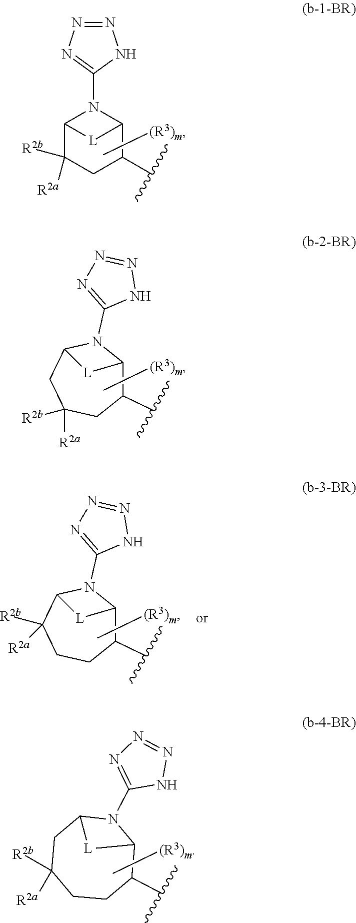

wherein L is a C₁₋₃ alkylene bridging group or C₁₋₃ haloalkylene bridging group.

In some embodiments, L is a C₁₋₃ alkylene bridging group.

In some embodiments, L is a C₁₋₃ haloalkylene bridging group.

In some embodiments, Ring B is a group of formula:

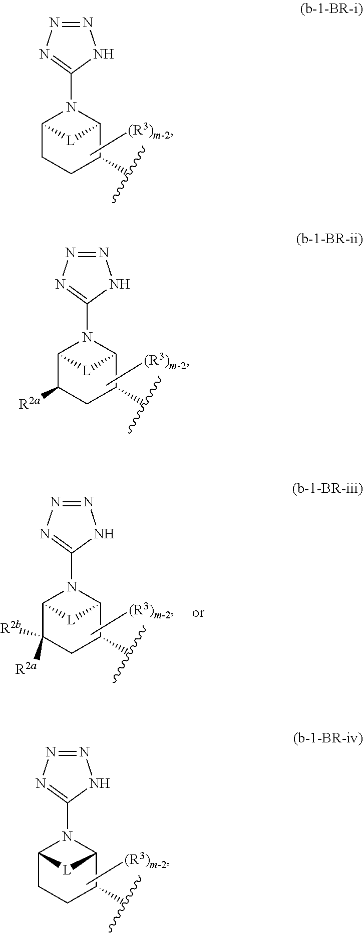

wherein L is a C₁₋₃ alkylene bridging group or C₁₋₃ haloalkylene bridging group.

In some embodiments, Ring B is a group of formula (b-i-BR-i), (b-i-BR-ii), or (b-i-BR-iii), wherein each instance of R^{2a} and R^{2b} is independently halo, C₁₋₆ alkyl, C₁₋₆haloalkyl, C₃-C₄ carbocyclyl, or 3-4 membered heterocyclyl.

In some embodiments, Ring B is a group of formula:

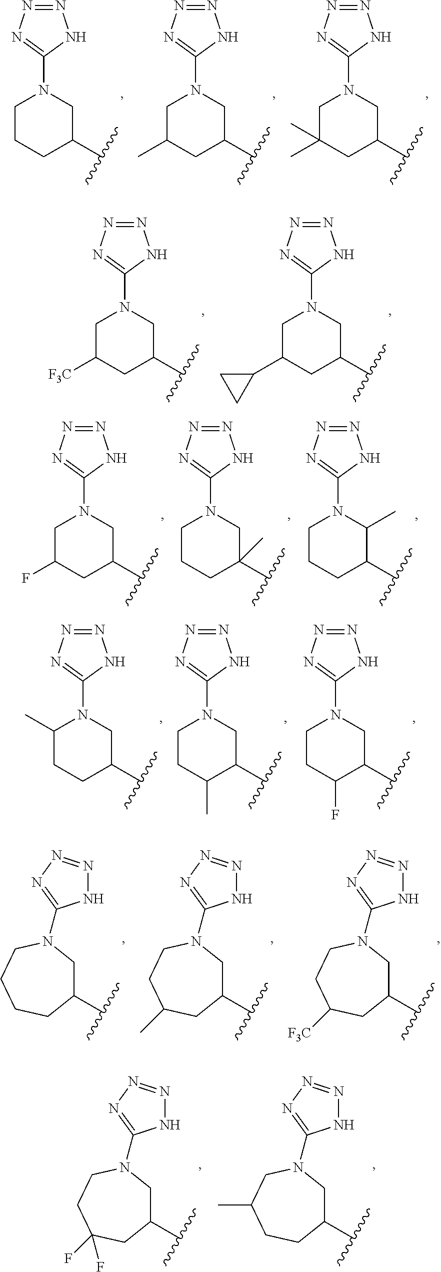

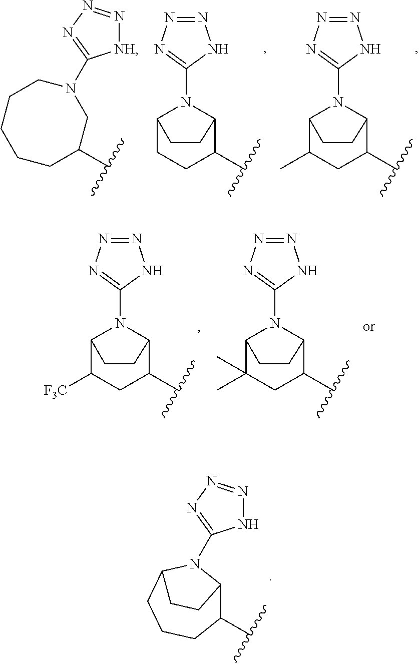

In some embodiments, Ring B is a group of formula:

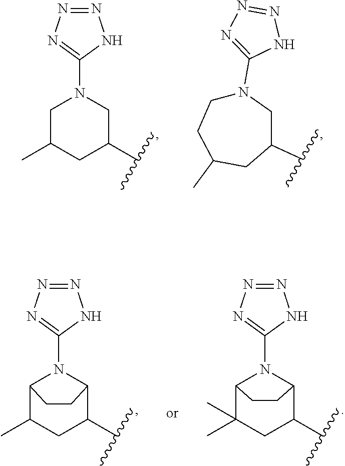 [SMILES: *C1CC(C)CN(c2nnn[nH]2)C1 ; *C1CC(C)CCN(c2nnn[nH]2)C1 ; *C1CC(C)C2CCC1N2c1nnn[nH]1 ; *C1CC(C)(C)C2CCC1N2c1nnn[nH]1]

In some embodiments, Ring B is a group of formula:

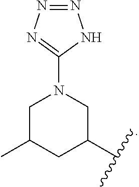 [SMILES: *C1CC(C)CN(c2nnn[nH]2)C1]

In some embodiments, Ring B is a group of formula:

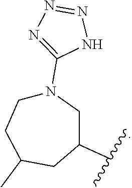 [SMILES: *C1CC(C)CCN(c2nnn[nH]2)C1]

In some embodiments, Ring B is a group of formula:

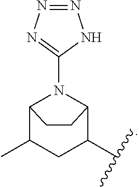 [SMILES: *C1CC(C)C2CCC1N2c1nnn[nH]1]

In some embodiments, Ring B is a group of formula:

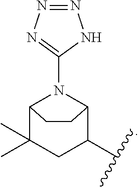 [SMILES: *C1CC(C)(C)C2CCC1N2c1nnn[nH]1]

In some embodiments, Ring B is a group of formula:

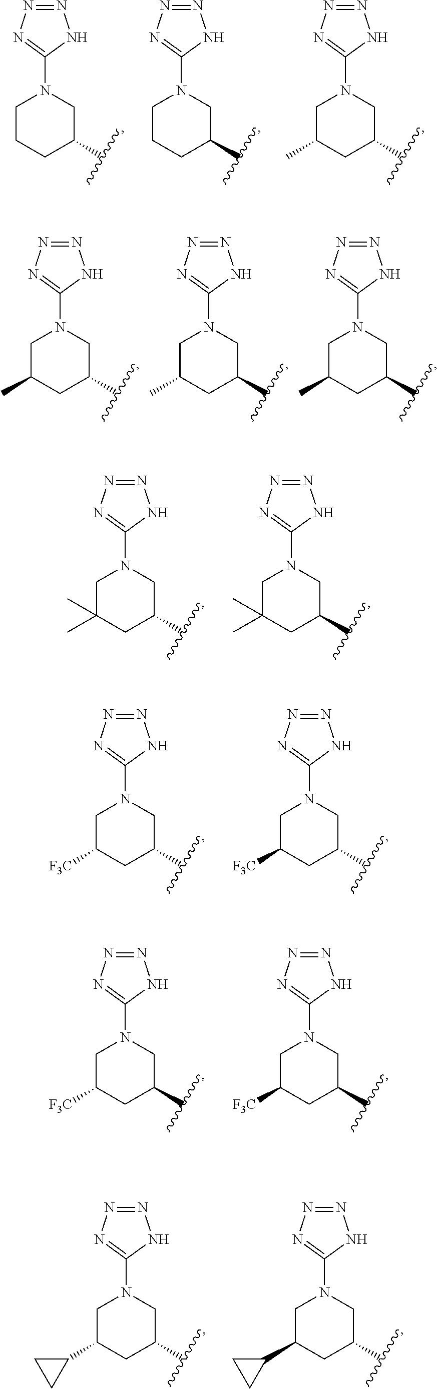

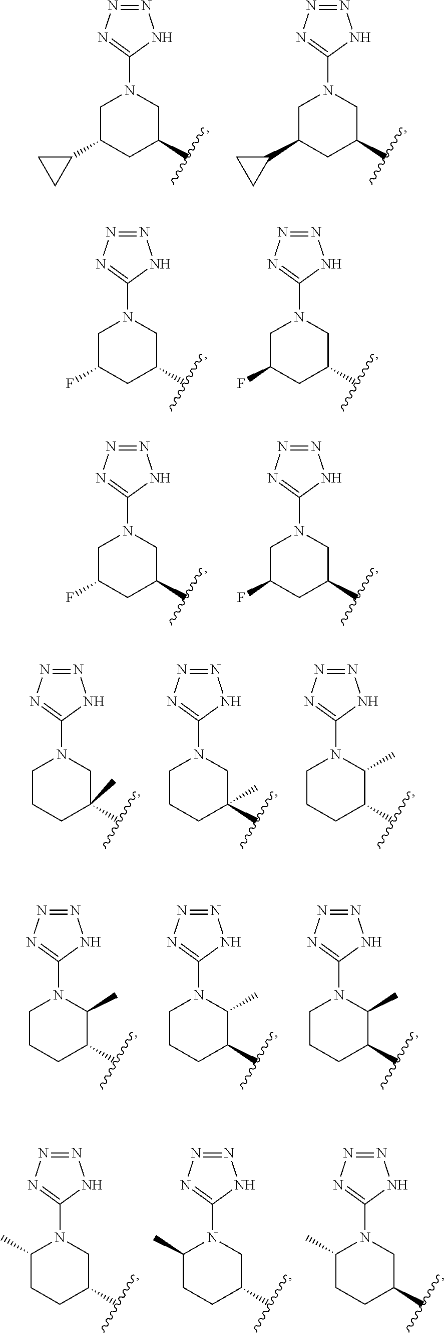

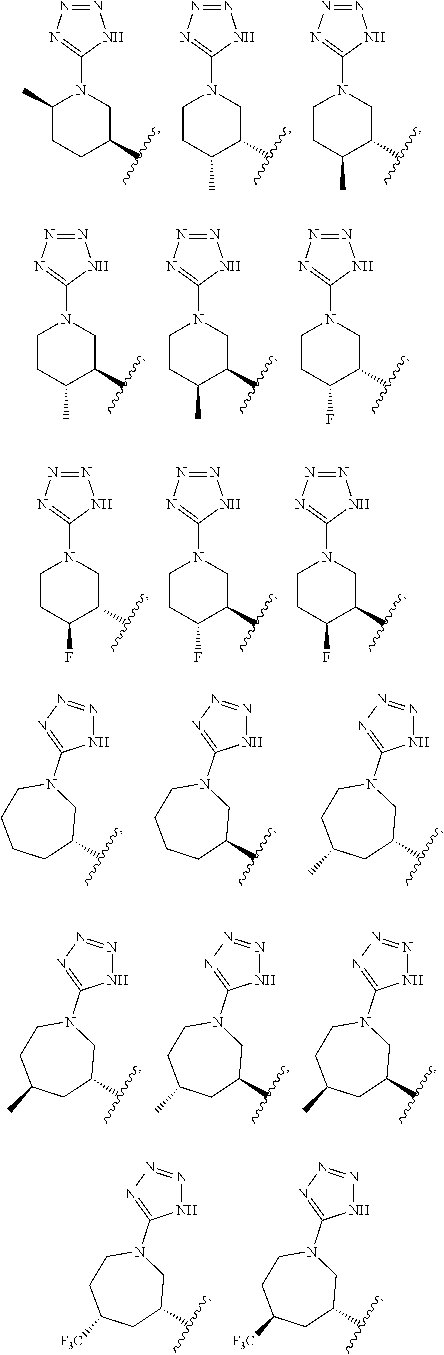

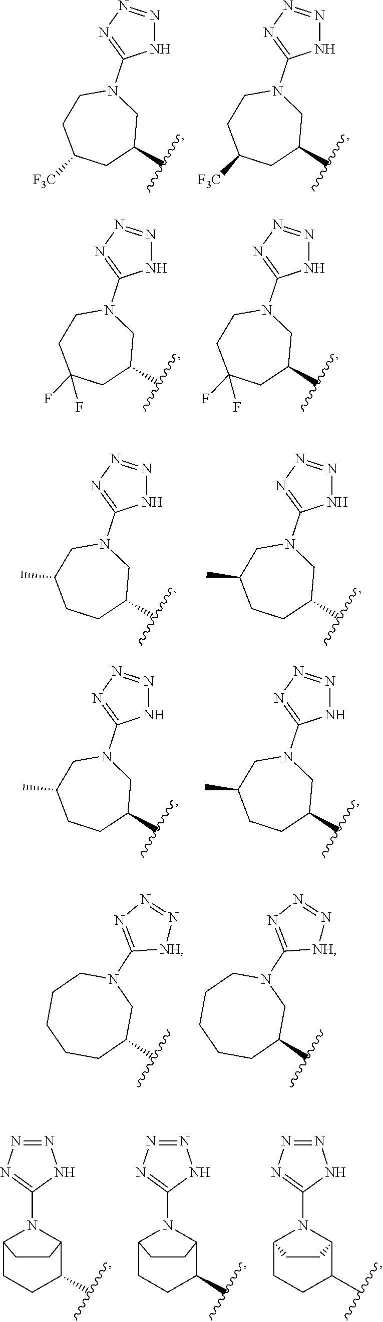

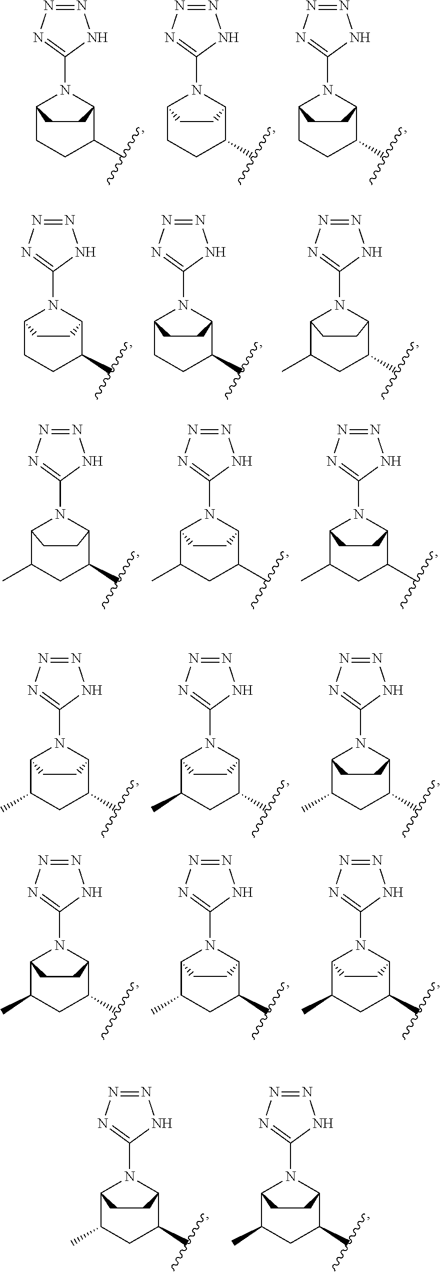

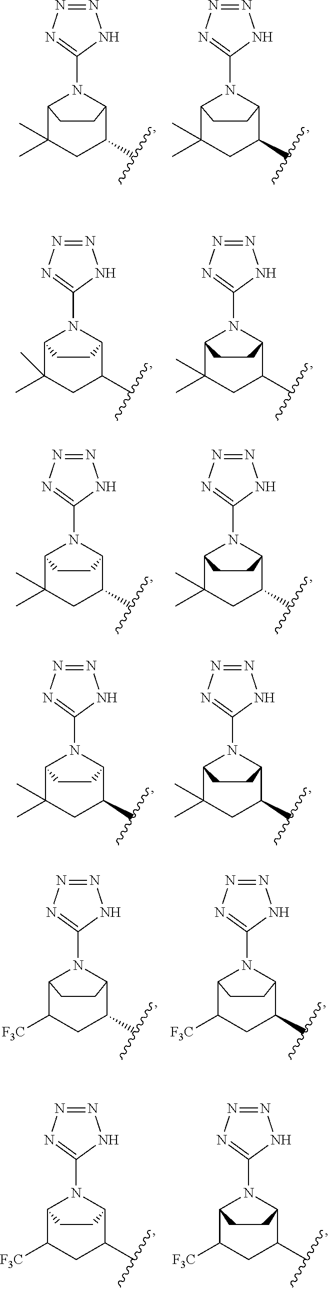

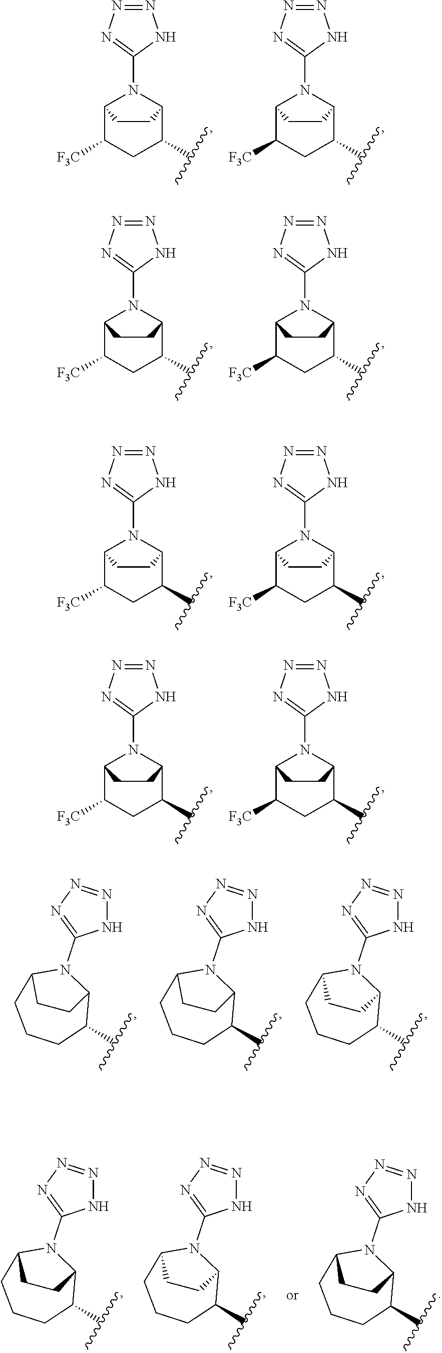

In some embodiments, Ring B is a group of formula:

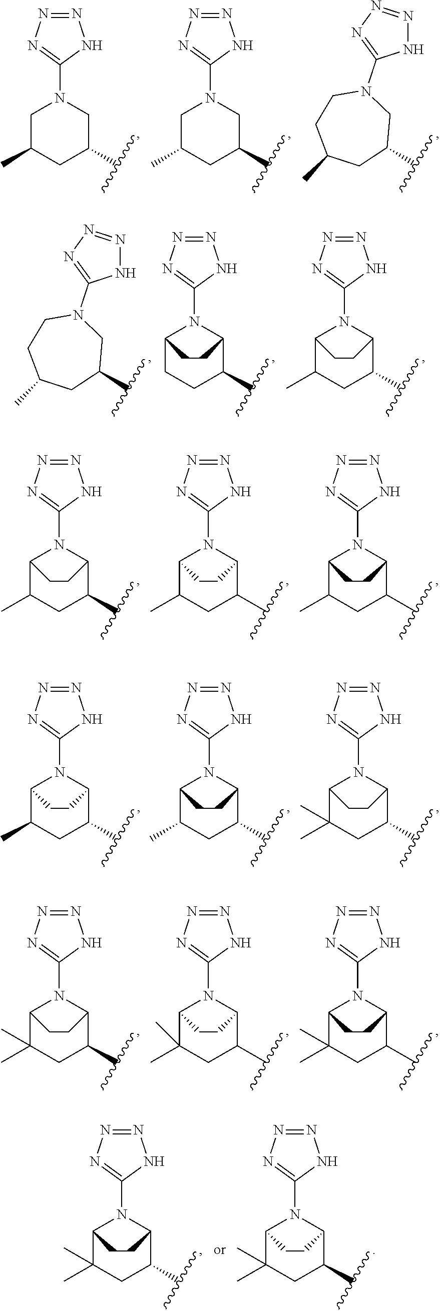

In some embodiments, Ring B is a group of formula:

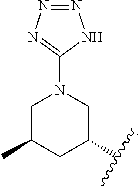 [SMILES: *[C@@H]1C[C@@H](C)CN(c2nnn[nH]2)C1]

In some embodiments, Ring B is a group of formula:

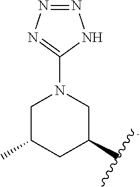 [SMILES: *[C@H]1C[C@H](C)CN(c2nnn[nH]2)C1]

In some embodiments, Ring B is a group of formula:

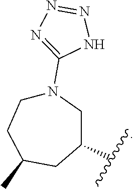 [SMILES: *[C@@H]1C[C@@H](C)CCN(c2nnn[nH]2)C1]

In some embodiments, Ring B is a group of formula:

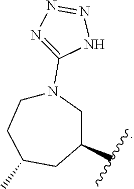 [SMILES: *[C@H]1C[C@H](C)CCN(c2nnn[nH]2)C1]

In some embodiments, Ring B is a group of formula:

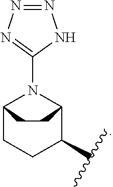 [SMILES: *[C@H]1CC[C@H]2CC[C@@H]1N2c1nnn[nH]1]

In some embodiments, Ring B is a group of formula:

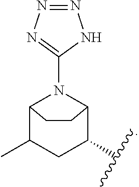 [SMILES: *[C@@H]1CC(C)C2CCC1N2c1nnn[nH]1]

In some embodiments, Ring is a group of formula:

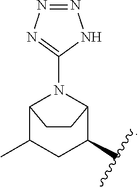 [SMILES: *[C@H]1CC(C)C2CCC1N2c1nnn[nH]1]

In some embodiments, Ring B is a group of formula:

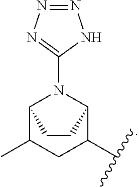 [SMILES: *C1CC(C)[C@@H]2CC[C@H]1N2c1nnn[nH]1]

In some embodiments, Ring B is a group of formula:

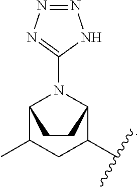 [SMILES: *C1CC(C)[C@H]2CC[C@@H]1N2c1nnn[nH]1]

In some embodiments, Ring B is a group of formula:

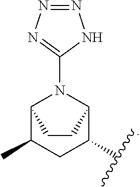 [SMILES: *[C@@H]1C[C@@H](C)[C@@H]2CC[C@H]1N2c1nnn[nH]1]

In some embodiments, Ring B is a group of formula:

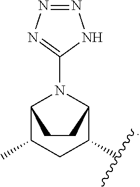 [SMILES: *[C@@H]1C[C@H](C)[C@H]2CC[C@@H]1N2c1nnn[nH]1]

In some embodiments, Ring B is a group of formula:

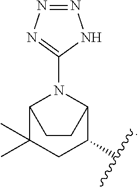 [SMILES: *[C@@H]1CC(C)(C)C2CCC1N2c1nnn[nH]1]

In some embodiments, Ring B is a group of formula:

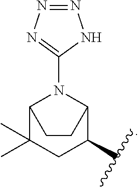 [SMILES: *[C@H]1CC(C)(C)C2CCC1N2c1nnn[nH]1]

In some embodiments, Ring B is a group of formula:

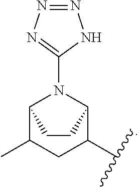 [SMILES: *C1CC(C)[C@@H]2CC[C@H]1N2c1nnn[nH]1]

In some embodiments, Ring B is a group of formula:

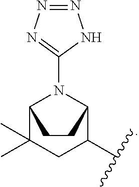 [SMILES: *C1CC(C)(C)[C@H]2CC[C@@H]1N2c1nnn[nH]1]

In some embodiments, Ring B is a group of formula:

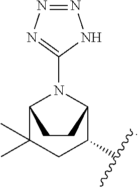 [SMILES: *[C@@H]1CC(C)(C)[C@H]2CC[C@@H]1N2c1nnn[nH]1]

In some embodiments, Ring B is a group of formula:

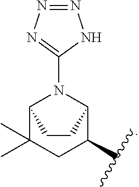 [SMILES: *[C@H]1CC(C)(C)[C@@H]2CC[C@H]1N2c1nnn[nH]1]

### (c) Subgenera

It is understood that, for a compound of the present disclosure, variables Ring A, G₁, G₂, G₃, G₄, R¹, R^{G1}, R^{G2}, R^{G3}, R^{G4}, R^{G5}, R^{G6}, R^{G7}, Ring B, n, p, m, R³, R^{2a}, and R^{2b} can each be, where applicable, selected from the groups described herein, and any group described herein for any of variables Ring A, G₁, G₂, G₃, G₄, R¹, R^{G1}, R^{G2}, R^{G3}, R^{G4}, R^{G5}, R^{G6}, R^{G7}, Ring B, n, p, m, R³, R^{2a}, and R^{2b} can be combined, where applicable, with any group described herein for one or more of the remainder of variables Ring A, G₁, G₂, G₃, G₄, R, R^{G1}, R^{G2}, R^{G3}, R^{G4}, R^{G5}, R^{G6}, R^{G7}, Ring B, n, p, m, R³, R^{2a}, and R^{2b}. Additional exemplary combinations of the above described embodiments are further contemplated herein.

For example, in certain embodiments, provided is a compound of Formula (I-A′), (I-A-a), (I-A′-Bridge), (I-A′″-Bridge), (I-A-Bridge-a), or (I-A-Bridge-c):

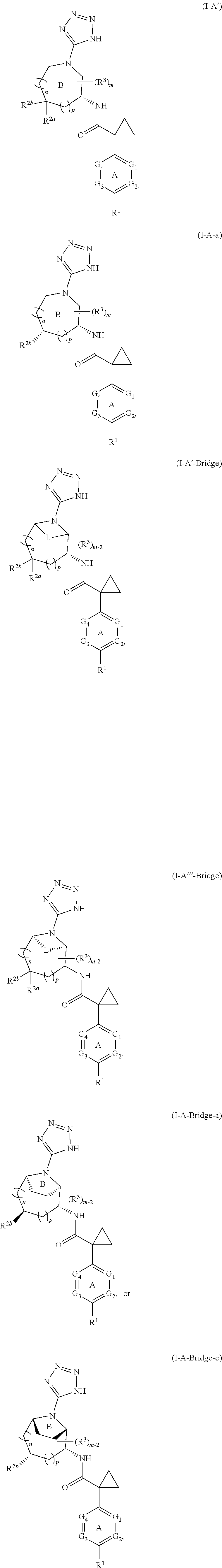

or a pharmaceutically acceptable salt or tautomer thereof.

In some embodiments of Formula (I-A′), (I-A-a), (I-A′-Bridge), (I-A′″-Bridge), (I-A-Bridge-a), or (I-A-Bridge-c), G₁ is CH, G₂ is CH, G₃ is CH, and G₄ is CH. In some embodiments of Formula (I-A′), (I-A-a), (I-A′-Bridge), (I-A′″-Bridge), (I-A-Bridge-a), or (I-A-Bridge-c), G₁ is CR^{G1}, G₂ is CH, G₃ is CH, and G₄ is CH. In some embodiments of Formula (I-A′), (I-A-a), (I-A′-Bridge), (I-A′″-Bridge), (I-A-Bridge-a), or (I-A-Bridge-c), G₁ is CH, G₂ is CR^{G2}, G₃ is CH, and G₄ is CH. In some embodiments of Formula (I-A′), (I-A-a), (I-A′-Bridge), (I-A′″-Bridge), (I-A-Bridge-a), or (I-A-Bridge-c), G₁ is CR^{G1}, G₂ is CR^{G2}, G₃ is CH, and G₄ is CH. In some embodiments of Formula (I-A′), (I-A-a), (I-A′-Bridge), (I-A′″-Bridge), (I-A-Bridge-a), or (I-A-Bridge-c), G₁ is CR^{G1}, G₂ is CH, G₃ is CR^{G3}, and G₄ is CH. In some embodiments of Formula (I-A′), (I-A-a), (I-A′-Bridge), (I-A′″-Bridge), (I-A-Bridge-a), or (I-A-Bridge-c), G₁ is CR^{G1}, G₂ is CR^{G2}, G₃ is CR^{G3}, and G₄ is CR^{G4}.

In some embodiments of Formula (I-A′), (I-A-a), (I-A′-Bridge), (I-A′″-Bridge), (I-A-Bridge-a), or (I-A-Bridge-c), and any of the above described embodiments of this section, R^{G1} is halo or —OR^{G6} and R¹ is halo, C₁₋₆haloalkyl, C₁₋₆alkyl, C₃-C₄ carbocyclyl, —OR^{G5}. In some embodiments, R^{G1} is halo and R¹ is —OR^{G5}. In some embodiments, R^{G1} is halo and R¹ is halo. In some embodiments, R^{G1} is halo and R¹ is C₁₋₆ haloalkyl. In some embodiments, R^{G1} is —OR^{G6} and R¹ is halo. In some embodiments, R^{G1} is —OR^{G6} and R¹ is C₁₋₆ haloalkyl. In some embodiments, R^{G1} is —OR^{G6} and R¹ is C₁₋₆ alkyl. In some embodiments, R^{G1} is —OR^{G6} and R¹ is C₃-C₄ carbocyclyl. In some embodiments, R^{G1} is —OR^{G6} and R¹ is —OR^{G5}

In some embodiments of Formula (I-A′), (I-A-a), (I-A′-Bridge), (I-A′″-Bridge), (I-A-Bridge-a), or (I-A-Bridge-c), and any of the above described embodiments of this section, R^{G2} is halo or C₁₋₆ alkyl and R¹ is halo, C₁₋₆ haloalkyl, or —OR^{G5}. In some embodiments, R^{G2} is halo and R¹ is halo. In some embodiments, R^{G2} is halo and R¹ is C₁₋₆haloalkyl. In some embodiments, R^{G2} is halo and R¹ is —OR^{G5}. In some embodiments, R^{G2} is —OR^{G6} and R¹ is C₁₋₆ haloalkyl. In some embodiments, R^{G2} is C₁₋₆ alkyl and R¹ is C₁₋₆haloalkyl.

In some embodiments of Formula (I-A′), (I-A-a), (I-A′-Bridge), (I-A′″-Bridge), (I-A-Bridge-a), or (I-A-Bridge-c), and any of the above described embodiments of this section, R^{G1} is halo or —OR^{G6} and R^{G2} is halo or C₁₋₆ alkyl. In some embodiments, R^{G1} is halo and R^{G2} is halo. In some embodiments, R^{G1} is halo, R^{G2} is halo, and R¹ is halo. In some embodiments, R^{G1} is halo, R^{G2} is halo, and R¹ is C₁₋₆ haloalkyl. In some embodiments, R^{G1} is —OR^{G6} and R^{G2} is halo. In some embodiments, R^{G1} is —OR^{G6}, R^{G2} is halo, and R¹ is halo. In some embodiments, R^{G1} is —OR^{G6}, R^{G2} is halo, and R¹ is C₁₋₆ alkyl. In some embodiments, R^{G1} is —OR^{G6}, R^{G2} is halo, and R¹ is —OR^{G5}. In some embodiments, R^{G1} is —OR^{G6}, R^{G2} is halo, and R¹ is C₁₋₆haloalkyl. In some embodiments, R^{G1} is —OR^{G6}, R^{G2} is halo, and R¹ is C₃-C₄ carbocyclyl. In some embodiments, R^{G1} is —OR^{G6} and R^{G2} is C₁₋₆ alkyl. In some embodiments, R^{G1} is —OR^{G5}, R^{G2} is C₁₋₆ alkyl, and R¹ is halo.

In some embodiments of Formula (I-A′), (I-A-a), (I-A′-Bridge), (I-A′″-Bridge), (I-A-Bridge-a), or (I-A-Bridge-c), and any of the above described embodiments of this section, R^{G1} is halo or —OR^{G6} and R^{G3} is halo. In some embodiments, R^{G1} is halo and R^{G3} is halo. In some embodiments, R^{G1} is halo, R^{G3} is halo, and R¹ is C₁₋₆haloalkyl. In some embodiments, R^{G1} is —OR^{G6} and R^{G3} is halo. In some embodiments, R^{G1} is —OR^{G6}, R^{G3} is halo, and R¹ is halo.

In some embodiments of Formula (I-A′), (I-A-a), (I-A′-Bridge), (I-A′″-Bridge), (I-A-Bridge-a), or (I-A-Bridge-c), and any of the above described embodiments of this section, R^{G1} is halo or —OR^{G6} and R^{G4} is C₁₋₆ alkyl. In some embodiments, R^{G1} is —OR^{G6} and R^{G4} is C₁₋₆ alkyl. In some embodiments, R^{G1} is —OR^{G6}, R^{G4} is C₁₋₆ alkyl, and R¹ is halo.

In some embodiments of Formula (I-A′), (I-A-a), (I-A′-Bridge), (I-A′″-Bridge), (I-A-Bridge-a), or (I-A-Bridge-c), and any of the above described embodiments of this section, n is 0 and p is 1. In some embodiments, n is 0, p is 1, and at least one of R^{2a} and R^{2b} is C₁₋₆ alkyl. In some embodiments, n is 0, p is 1, and at least one of R^{2a} and R^{2b} is halo. In some embodiments, n is 0, p is 1, and at least one of R^{2a} and R^{2b} is C₁₋₆ haloalkyl. In some embodiments, n is 0, p is 1, and at least one R³ is C₁₋₆ alkyl. In some embodiments, n is 0, p is 1, and at least one R³ is halo. In some embodiments, n is 0, p is 1, and two R³ groups, together with the atoms to which they are attached, join to form a C₁₋₃ alkylene (e.g., ethylene bridge). In some embodiments, n is 0, p is 1, two R³ groups, together with the atoms to which they are attached, join to form a C₁₋₃ alkylene (e.g., ethylene bridge), and at least one of R^{2a} and R^{2b} is C₁₋₆ alkyl.

In some embodiments of Formula (I-A′), (I-A-a), (I-A′-Bridge), (I-A′″-Bridge), (I-A-Bridge-a), or (I-A-Bridge-c), and any of the above described embodiments of this section, n is 1 and p is 1. In some embodiments, n is 1, p is 1, and at least one of R^{2a} and R^{2b} is C₁₋₆haloalkyl. In some embodiments, n is 1, p is 1, and at least one of R^{2a} and R^{2b} is C₁₋₆ alkyl. In some embodiments, n is 1, p is 1, and at least one of R^{2a} and R^{2b} is C₃-C₄ carbocyclyl. In some embodiments, n is 1, p is 1, and at least one of R^{2a} and R^{2b} is halo. In some embodiments, n is 1, p is 1, R^{2a} is H, and R^{2b} is H. In some embodiments, n is 1, p is 1, and two R³ groups, together with the atoms to which they are attached, join to form a C₁₋₃ alkylene (e.g., ethylene bridge). In some embodiments, n is 1, p is 1, two R³ groups, together with the atoms to which they are attached, join to form a C₁₋₃ alkylene (e.g., ethylene bridge), and at least one of R^{2a} and R^{2b} is C₁₋₆ alkyl.

In some embodiments of Formula (I-A′), (I-A-a), (I-A′-Bridge), (I-A′″-Bridge), (I-A-Bridge-a), or (I-A-Bridge-c), Ring A is a ring system wherein:
R¹ is halo, C₁₋₆ alkyl, C₁₋₆ haloalkyl, —OR^{G5}, —SR^{G5}, C₃-C₄ carbocyclyl, wherein the carbocyclyl is independently substituted with 0, 1, 2, or 3 halo, C₁₋₆ alkyl, C₁₋₆ haloalkyl, —OR^{G5}, —SR^{G5}, or —N(R^{G5})₂
R^{G1}, R^{G2}, R^{G3}, and R^{G4} are each independently selected from the group consisting of hydrogen, halo, C₁₋₆ alkyl, and —OR^{G6}; and
R^{G5} and R^{G6} are each independently hydrogen, C₁₋₆ alkyl, or C₁₋₆ haloalkyl.

In some embodiments of Formula (I-A′), (I-A-a), (I-A′-Bridge), (I-A′″-Bridge), (I-A-Bridge-a), or (I-A-Bridge-c), Ring A is a ring system wherein: R¹ is Cl, Br, methyl, isopropyl, cyclopropyl, difluorocyclopropyl, cyclobutyl, OCF₂H, OCF₃, CF₂H, CF₃, SCF₃, or SCF₂H; and
R^{G1}, R^{G2}, R^{G3}, and R^{G4} are each independently selected from the group consisting of hydrogen, F, Cl, methyl, OH, OCH₃, and OCF₂H.

In some embodiments of Formula (I-A′), (I-A-a), (I-A′-Bridge), (I-A′″-Bridge), (I-A-Bridge-a), or (I-A-Bridge-c), Ring B is a ring system wherein:
each instance of R^{2a} and R^{2b} is independently hydrogen, halo, C₁₋₆ alkyl, C₁₋₆ haloalkyl, or C₃-C₄ carbocyclyl; and
each instance of R³ is independently C₁₋₆ alkyl,
or two R³ groups, together with the atoms to which they are attached, may be joined to form a C₁₋₃ alkylene bridging group.

In some embodiments of Formula (I-A′), (I-A-a), (I-A′-Bridge), (I-A′″-Bridge), (I-A-Bridge-a), or (I-A-Bridge-c), Ring B is a ring system wherein:
each instance of R^{2a} and R^{2b} is independently hydrogen, F, CF₃, methyl or cyclopropyl; and
each instance of R³ is independently methyl,
or two R³ groups, together with the atoms to which they are attached, may be joined to form an ethylene bridging group.

In certain embodiments of Formula (I-A-a), wherein n is 0, p is 1, m is 0 (where R³ is absent), G₁ ism CR^{G1}, G₂ is CR^{G2}, G₃ is CH, and G₄ is CR^{G4}, provided is a compound of Formula (II-A-al):

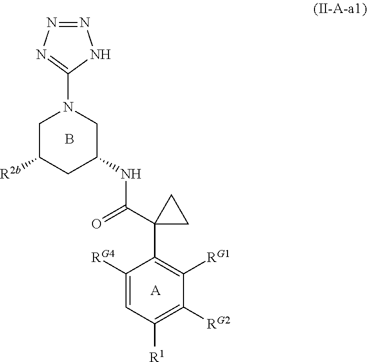

or a pharmaceutically acceptable salt or tautomer thereof. In certain embodiments, R^{2b} is not hydrogen. In certain embodiments, R^{2b} is methyl. In certain embodiments, R¹ is halogen, C₃carbocyclyl, C₁₋₃ alkyl, C₁₋₃ haloalkyl, or —OR⁵ wherein R^{G5} is C₁₋₃ alkyl or C₁₋₃ haloalkyl. In certain embodiments, R¹ is C₁₋₃ alkyl, C₁₋₃ haloalkyl or —OR^{G5} wherein R^{G5} is C₁₋₃ alkyl or C₁₋₃ haloalkyl. In certain embodiments, R^{G1} is hydrogen, —OH, or halogen (e.g., fluoro or chloro). In certain embodiments, R^{G1} is hydrogen, —OH or fluoro. In certain embodiments, R^{G1} is —OH or fluoro. In certain embodiments, R^{G2} is hydrogen, fluoro, or —OR^{G6} wherein R^{G6} is C₁₋₃ alkyl or C₁₋₃ haloalkyl. In certain embodiments, R^{G4} is hydrogen or fluoro. In certain embodiments, R^{G1} is —OH or fluoro, R^{G2} is hydrogen, R^{G4} is hydrogen or fluoro, and R¹ is C₁₋₃ haloalkyl or —OR^{G5} wherein R^{G5} is C₁₋₃ alkyl or C₁₋₃ haloalkyl.

In certain embodiments of Formula (I-B-a), wherein n is 1, p is 1, m is 0 (where R³ is absent), G₁ is CR^{G1}, G₂ is CR^{G2}, G₃ is CH, and G₄ is CR^{G4}, provided is a compound of Formula (II-B-a2):

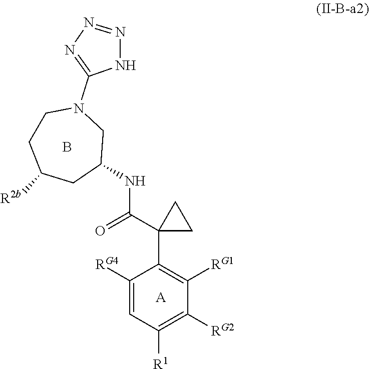

or a pharmaceutically acceptable salt or tautomer thereof. In certain embodiments, R^{2b} is not hydrogen. In certain embodiments, R^{2b} is methyl. In certain embodiments, R¹ is halogen, C₃carbocyclyl, C₁₋₃ alkyl, C₁₋₃ haloalkyl, or —OR^{G5} wherein R^{G5} is C₁₋₃ alkyl or C₁₋₃ haloalkyl. In certain embodiments, R¹ is C₁₋₃ alkyl, C₁₋₃ haloalkyl or —OR^{G5} wherein R^{G5} is C₁₋₃ alkyl or C₁₋₃ haloalkyl. In certain embodiments, R^{G1} is hydrogen, —OH, or halogen (e.g., fluoro or chloro). In certain embodiments, R^{G1} is hydrogen, —OH or fluoro. In certain embodiments, R^{G1} is —OH or fluoro. In certain embodiments, R^{G1} is hydrogen, fluoro, or —OR^{G6}wherein R^{G6} is C₁₋₃ alkyl or C₁₋₃ haloalkyl. In certain embodiments, R^{G4} is hydrogen or fluoro. In certain embodiments, R^{G1} is —OH or fluoro, R^{G2} is hydrogen, R^{G4} is hydrogen or fluoro, and R¹ is C₁₋₃ haloalkyl or —OR^{G5} wherein R^{G5} is C₁₋₃ alkyl or C₁₋₃ haloalkyl.

In certain embodiments of Formula (I-B-Bridge-a), wherein n is 0, p is 1, m is 0 (where an additional R³ is absent), G₁ is CR^{G1}, G₂ is CR^{G2}, G₃ is CH, and G₄ is CR^{G4}, provided is a compound of Formula (II-B-Bridge-a):

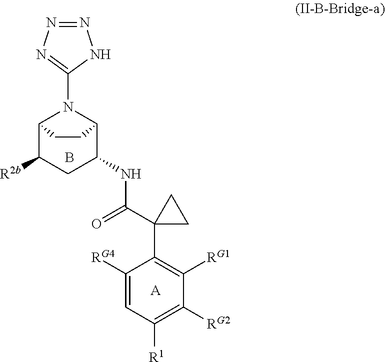

or a pharmaceutically acceptable salt or tautomer thereof. In certain embodiments, R^{2a} is not hydrogen. In certain embodiments, R^{2a} is methyl. In certain embodiments, R¹ is halogen, C₃carbocyclyl, C₁₋₃ alkyl, C₁₋₃ haloalkyl, or —OR^{G5} wherein R^{G5} is C₁₋₃ alkyl or C₁₋₃ haloalkyl. In certain embodiments, R¹ is C₁₋₃ alkyl, C₁₋₃ haloalkyl or —OR^{G5} wherein R^{G5} is C₁₋₃ alkyl or C₁₋₃ haloalkyl. In certain embodiments, R^{G1} is hydrogen, —OH, or halogen (e.g., fluoro or chloro). In certain embodiments, R^{G1} is hydrogen, —OH or fluoro. In certain embodiments, R^{G1} is —OH or fluoro. In certain embodiments, R^{G2} is hydrogen, fluoro, or —OR^{G6}wherein R^{G6} is C₁₋₃ alkyl or C₁₋₃ haloalkyl. In certain embodiments, R^{G4} is hydrogen or fluoro. In certain embodiments, R^{G1} is —OH or fluoro, R^{G2} is hydrogen, R^{G4} is hydrogen or fluoro, and R¹ is C₁₋₃ haloalkyl or —OR^{G5} wherein R^{G5} is C₁₋₃ alkyl or C₁₋₃ haloalkyl.

In certain embodiments of Formula (I-A-Bridge-c), wherein n is 0, p is 1, m is 0 (where R³ is absent), R^{2a} and R^{2b} are both hydrogen, G₁ is CR^{G1}, G₂ is CR^{G2}, G₃ is CH, and G₄ is CR^{G4}, provided is a compound of Formula (II-A-Bridge-c):

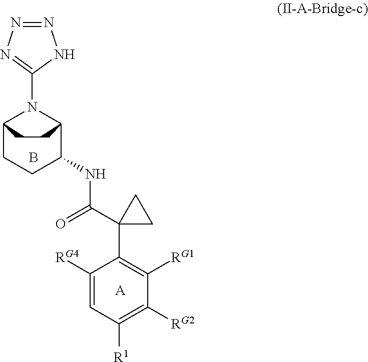

or a pharmaceutically acceptable salt or tautomer thereof. In certain embodiments, R¹ is halogen, C₃carbocyclyl, C₁₋₃ alkyl, C₁₋₃ haloalkyl, or —OR^{G5} wherein R^{G5} is C₁₋₃ alkyl or C₁₋₃ haloalkyl. In certain embodiments, R¹ is C₁₋₃ alkyl, C₁₋₃ haloalkyl or —OR^{G5} wherein R^{G5} is C₁₋₃ alkyl or C₁₋₃ haloalkyl. In certain embodiments, R^{G1} is hydrogen, —OH, or halogen (e.g., fluoro or chloro). In certain embodiments, R^{G1} is hydrogen, —OH or fluoro. In certain embodiments, R^{G1} is —OH or fluoro. In certain embodiments, R^{G2} is hydrogen, fluoro, or —OR^{G6} wherein R^{G6} is C₁₋₃ alkyl or C₁₋₃ haloalkyl. In certain embodiments, R^{G4} is hydrogen or fluoro. In certain embodiments, R^{G1} is —OH or fluoro, R^{G2} is hydrogen, R^{G4} is hydrogen or fluoro, and R¹ is C₁₋₃ haloalkyl or —OR^{G5} wherein R^{G5} is C₁₋₃ alkyl or C₁₋₃ haloalkyl.

In some embodiments, the compound of Formula (I-A) is selected from any one of the compounds of Tables 1, 2, or 3, or a pharmaceutically acceptable salt or tautomer thereof.

In some embodiments, the compound of Formula (I-A) is a pharmaceutically acceptable salt of any one of the compounds of Tables 1, 2, or 3, or tautomer thereof.

In some embodiments, the compound of Formula (I-A) is a free base selected from any one of the compounds of Tables 1, 2, or 3, or tautomer thereof.

The below Tables 1, 2, or 3 also provides the location of the compound in the Examples (Ex) by Example Number (Ex) or as provided in Table A (TA) of the Examples. The Asterix (*) next to the Compound Number (#) signifies that arbitrary stereochemistry has been assigned.

TABLE 1
Compounds of Formula (I-A)

| # | Ex | Structure/Name | # | Ex | Structure/Name |
|---|---|---|---|---|---|
| 1A* | 1 | 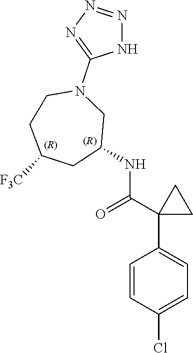 | 48A | TA | 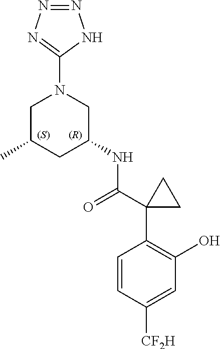 |
| | | N-((3R,5R)-1-(1H-tetrazol-5-yl)-5- | | | 1-(4-(difluoromethyl)-2- |
| | | (trifluoromethyl)azepan-3-yl)-1-(4- | | | hydroxyphenyl)-N-((3R,5S)-5-methyl-1- |
| | | chlorophenyl)cyclopropane-1- | | | (1H-tetrazol-5-yl)piperidin-3- |
| | | carboxamide | | | yl)cyclopropane-1-carboxamide |
| 1B* | 1 | 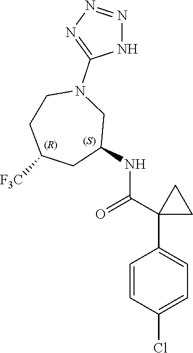 | 48B | TA | 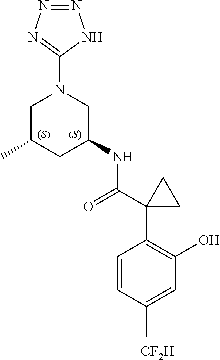 |
| | | N-((3S,5R)-1-(1H-tetrazol-5-yl)-5- | | | 1-(4-(difluoromethyl)-2- |
| | | (trifluoromethyl)azepan-3-yl)-1-(4- | | | hydroxyphenyl)-N-((3S,5S)-5-methyl-1- |
| | | chlorophenyl)cyclopropane-1- | | | (1H-tetrazol-5-yl)piperidin-3- |
| | | carboxamide | | | yl)cyclopropane-1-carboxamide |
| 1C* | 1 | 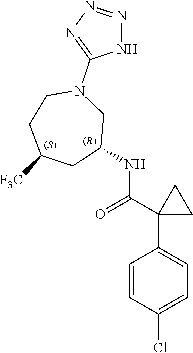 | 48C | TA | 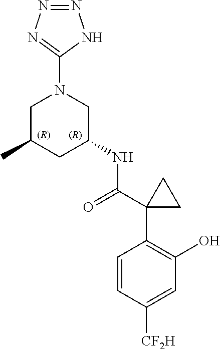 |
| | | N-((3R,5S)-1-(1H-tetrazol-5-yl)-5- | | | 1-(4-(difluoromethyl)-2- |
| | | (trifluoromethyl)azepan-3-yl)-1-(4- | | | hydroxyphenyl)-N-((3R,5R)-5-methyl- |
| | | chlorophenyl)cyclopropane-1- | | | 1-(1H-tetrazol-5-yl)piperidin-3- |
| | | carboxamide | | | yl)cyclopropane-1-carboxamide |
| 1D* | 1 | 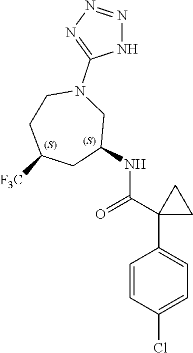 | 48D | TA | 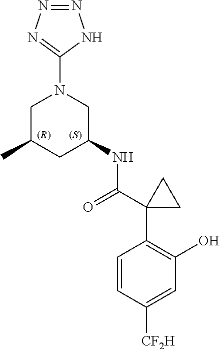 |
| | | N-((3S,5S)-1-(1H-tetrazol-5-yl)-5- | | | 1-(4-(difluoromethyl)-2- |
| | | (trifluoromethyl)azepan-3-yl)-1-(4- | | | hydroxyphenyl)-N-((3R,5S)-5-methyl-1- |
| | | chlorophenyl)cyclopropane-1- | | | (1H-tetrazol-5-yl)piperidin-3- |
| | | carboxamide | | | yl)cyclopropane-1-carboxamide |
| 2A* | 2 | 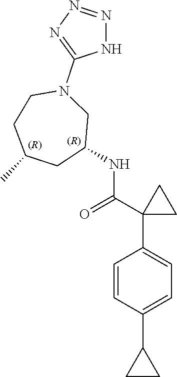 | 49A | TA | 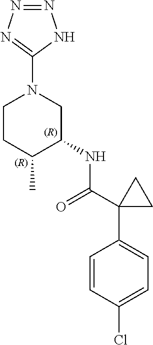 |
| | | 1-(4-cyclopropylphenyl)-N- | | | 1-(4-chlorophenyl)-N-[(3R,4R)-4- |
| | | ((3R,5R)-5-methyl-1-(1H-tetrazol- | | | methyl-1-(1H-tetrazol-5-yl)-3- |
| | | 5-yl)azepan-3-yl)cyclopropane-1- | | | piperidyl\|cyclopropanecarboxamide |
| | | carboxamide | | | |
| 2B* | 2 | 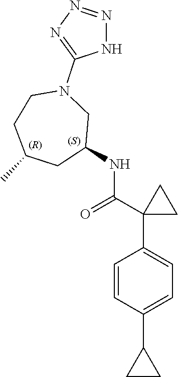 | 49B | TA | 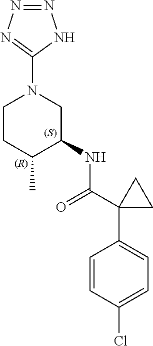 |
| | | 1-(4-cyclopropylphenyl)-N- | | | 1-(4-chlorophenyl)-N-[(3S,4R)-4- |
| | | ((3S,5R)-5-methyl-1-(1H-tetrazol- | | | methyl-1-(1H-tetrazol-5-yl)-3- |
| | | 5-yl)azepan-3-yl)cyclopropane-1- | | | piperidyl]cyclopropanecarboxamide |
| | | carboxamide | | | |
| 2C* | 2 | 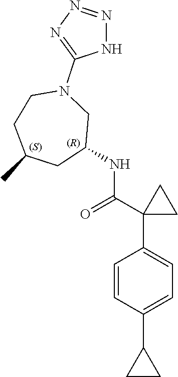 | 49C | TA | 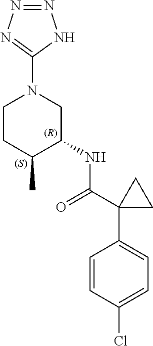 |
| | | 1-(4-cyclopropylphenyl)-N- | | | 1-(4-chlorophenyl)-N-[(3R,4S)-4- |
| | | ((3R,5S)-5-methyl-1-(1H-tetrazol- | | | methyl-1-(1H-tetrazol-5-yl)-3- |
| | | 5-yl)azepan-3-yl)cyclopropane-1- | | | piperidyl]cyclopropanecarboxamide |
| | | carboxamide | | | |
| 2D* | 2 | 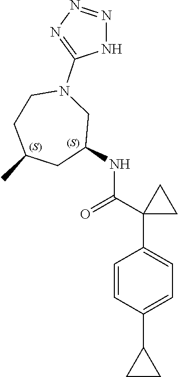 | 49D | TA | 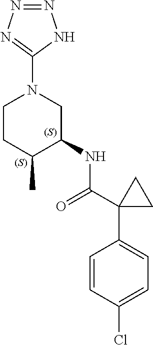 |
| | | 1-(4-cyclopropylphenyl)-N- | | | 1-(4-chlorophenyl)-N-[(3S,4S)-4- |
| | | ((3S,5S)-5-methyl-1-(1H-tetrazol- | | | methyl-1-(1H-tetrazol-5-yl)-3- |
| | | 5-yl)azepan-3-yl)cyclopropane-1- | | | piperidyl]cyclopropanecarboxamide |
| | | carboxamide | | | |
| 3A* | 3 | 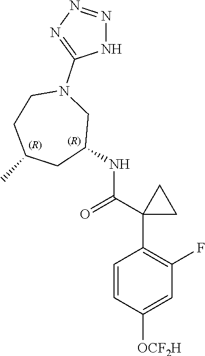 | 50A | TA | 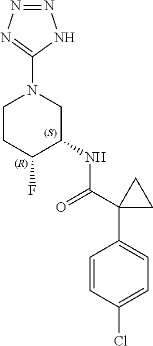 |
| | | 1-(4-(difluoromethoxy)-2- | | | 1-(4-chlorophenyl)-N-[(3S,4R)-4-fluoro- |
| | | fluorophenyl)-N-((3R,5R)-5- | | | 1-(1H-tetrazol-5-yl)-3- |
| | | methyl-1-(1H-tetrazol-5-yl)azepan- | | | piperidyl]cyclopropanecarboxamide |
| | | 3-yl)cyclopropane-1-carboxamide | | | |
| 3B* | 3 | 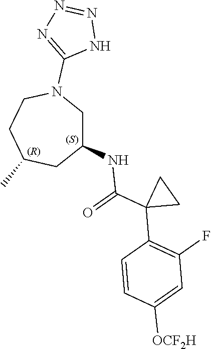 | 50B | TA | 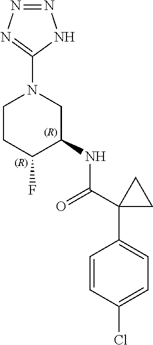 |
| | | 1-(4-(difluoromethoxy)-2- | | | 1-(4-chlorophenyl)-N-[(3R,4R)-4- |
| | | fluorophenyl)-N-((3S,5R)-5- | | | fluoro-1-(1H-tetrazol-5-yl)-3- |
| | | methyl-1-(1H-tetrazol-5-yl)azepan- | | | piperidyl]cyclopropanecarboxamide |
| | | 3-yl)cyclopropane-1-carboxamide | | | |
| 3C* | 3 | 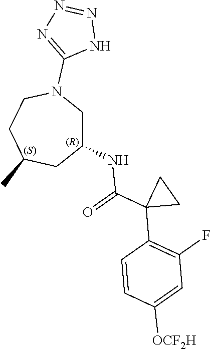 | 50C | TA | 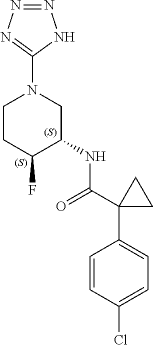 |
| | | 1-(4-(difluoromethoxy)-2- | | | 1-(4-chlorophenyl)-N-[(3S,4S)-4-fluoro- |
| | | fluorophenyl)-N-((3R,5S)-5- | | | 1-(1H-tetrazol-5-yl)-3- |
| | | methyl-1-(1H-tetrazol-5-yl)azepan- | | | piperidyl\|cyclopropanecarboxamide |
| | | 3-yl)cyclopropane-1-carboxamide | | | |
| 3D* | 3 | 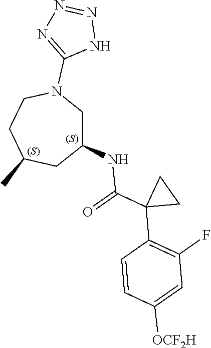 | 50D | TA | 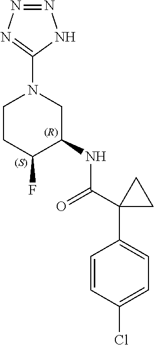 |
| | | 1-(4-(difluoromethoxy)-2- | | | 1-(4-chlorophenyl)-N-[(3R,4S)-4-fluoro- |
| | | fluorophenyl)-N-((3S,5S)-5- | | | 1-(1H-tetrazol-5-yl)-3- |
| | | methyl-1-(1H-tetrazol-5-yl)azepan- | | | piperidyl]cyclopropanecarboxamide |
| | | 3-yl)cyclopropane-1-carboxamide | | | |
| 4A* | 4 | 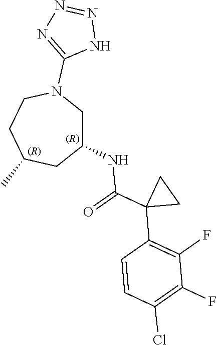 | 51A | TA | 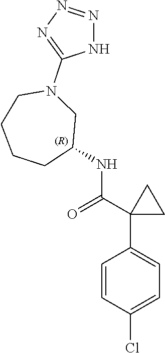 |
| | | 1-(4-chloro-2,3-difluorophenyl)-N- | | | 1-(4-chlorophenyl)-N-[(3R)-1-(1H- |
| | | ((3R,5R)-5-methyl-1-(1H-tetrazol- | | | tetrazol-5-yl)azepan-3- |
| | | 5-yl)azepan-3-yl)cyclopropane-1- | | | yl]cyclopropanecarboxamide |
| | | carboxamide | | | |
| 4B* | 4 | 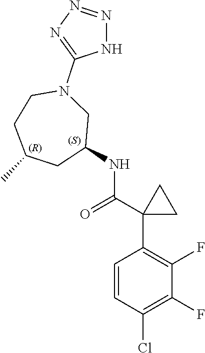 | 51B | TA | 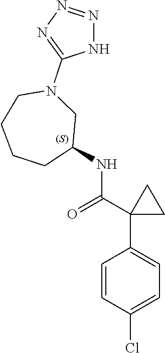 |
| | | 1-(4-chloro-2,3-difluorophenyl)-N- | | | 1-(4-chlorophenyl)-N-[(3S)-1-(1H- |
| | | ((3S,5R)-5-methyl-1-(1H-tetrazol- | | | tetrazol-5-yl)azepan-3- |
| | | 5-yl)azepan-3-yl)cyclopropane-1- | | | yl]cyclopropanecarboxamide |
| | | carboxamide | | | |
| 4C* | 4 | 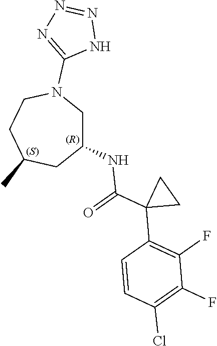 | 52A | TA | 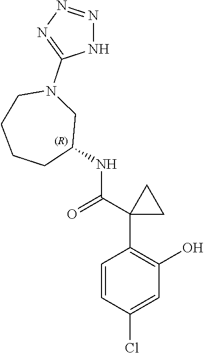 |
| | | 1-(4-chloro-2,3-difluorophenyl)-N- | | | (R)-N-(1-(1H-tetrazol-5-yl)azepan-3-yl)- |
| | | ((3R,5S)-5-methyl-1-(1H-tetrazol- | | | 1-(4-chloro-2- |
| | | 5-yl)azepan-3-yl)cyclopropane-1- | | | hydroxyphenyl)cyclopropane-1- |
| | | carboxamide | | | carboxamide |
| 4D* | 4 | 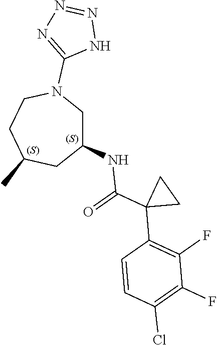 | 52B | TA | 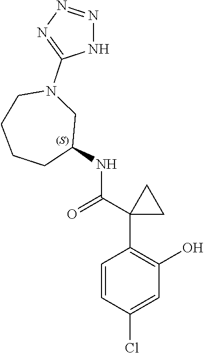 |
| | | 1-(4-chloro-2,3-difluorophenyl)-N- | | | (S)-N-(1-(1H-tetrazol-5-yl)azepan-3-yl)- |
| | | ((3S,5S)-5-methyl-1-(1H-tetrazol- | | | 1-(4-chloro-2- |
| | | 5-yl)azepan-3-yl)cyclopropane-1- | | | hydroxyphenyl)cyclopropane-1- |
| | | carboxamide | | | carboxamide |
| 5A* | 5 | 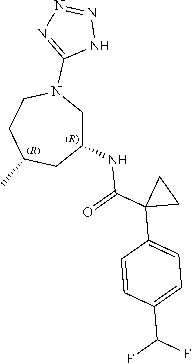 | Rac-53 | TA | 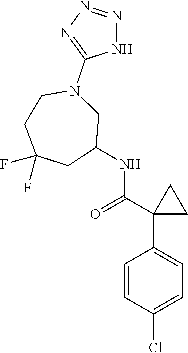 [SMILES: O=C(NC1CN(c2nnn[nH]2)CCC(F)(F)C1)C1(c2ccc(Cl)cc2)CC1] |
| | | 1-(4-(difluoromethyl)phenyl)-N- | | | 1-(4-chlorophenyl)-N-(5,5-difluoro-1- |
| | | ((3R,5R)-5-methyl-1-(1H-tetrazol- | | | (1H-tetrazol-5-yl)azepan-3- |
| | | 5-yl)azepan-3-yl)cyclopropane-1- | | | yl)cyclopropane-1-carboxamide |
| | | carboxamide | | | |
| 5B* | 5 | 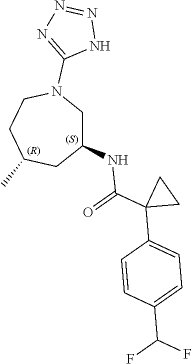 | 53A* | TA | 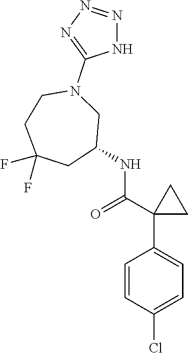 [SMILES: O=C(N[C@H]1CN(c2nnn[nH]2)CCC(F)(F)C1)C1(c2ccc(Cl)cc2)CC1] |
| | | 1-(4-(difluoromethyl)phenyl)-N- | | | (R)- 1-(4-chlorophenyl)-N-(5,5-difluoro- |
| | | ((3S,5R)-5-methyl-1-(1H-tetrazol- | | | 1-(1H-tetrazol-5-yl)azepan-3- |
| | | 5-yl)azepan-3-yl)cyclopropane-1- | | | yl)cyclopropane-1-carboxamide |
| | | carboxamide | | | |
| 5C* | 5 | 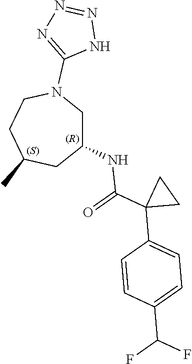 | 53B* | TA | 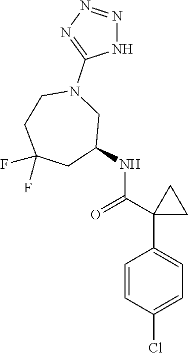 [SMILES: O=C(N[C@@H]1CN(c2nnn[nH]2)CCC(F)(F)C1)C1(c2ccc(Cl)cc2)CC1] |
| | | 1-(4-(difluoromethyl)phenyl)-N- | | | (S)- 1-(4-chlorophenyl)-N-(5,5-difluoro- |
| | | ((3R,5S)-5-methyl-1-(1H-tetrazol- | | | 1-(1H-tetrazol-5-yl)azepan-3- |
| | | 5-yl)azepan-3-yl)cyclopropane-1- | | | yl)cyclopropane-1-carboxamide |
| | | carboxamide | | | |
| 5D* | 5 | 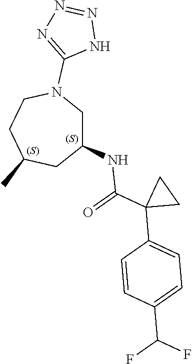 | Rac-54 | TA | 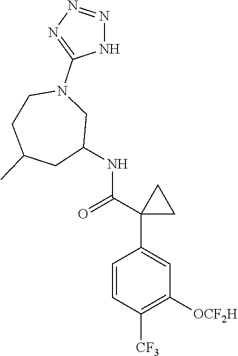 |
| | | 1-(4-(difluoromethyl)phenyl)-N- | | | 1-(3-(difluoromethoxy)-4- |
| | | ((3S,5S)-5-methyl-1-(1H-tetrazol- | | | (trifluoromethyl)phenyl)-N-(5-methyl-1- |
| | | 5-yl)azepan-3-yl)cyclopropane-1- | | | (1H-tetrazol-5-yl)azepan-3- |
| | | carboxamide | | | yl)cyclopropane-1-carboxamide |
| 6A* | 6 | 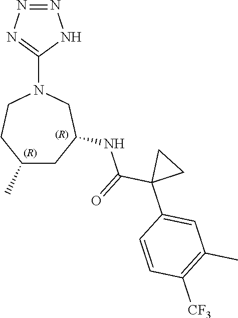 | 54A* | TA | 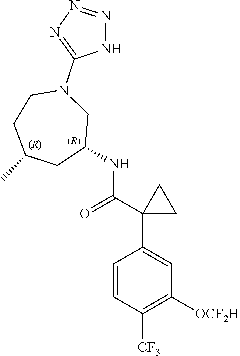 |
| | | N-((3R,5R)-5-methyl-1-(1H- | | | 1-(3-(difluoromethoxy)-4- |
| | | tetrazol-5-yl)azepan-3-yl)-1-(3- | | | (trifluoromethyl)phenyl)-N-((3R,5R)-5- |
| | | methyl-4- | | | methyl-1-(1H-tetrazol-5-yl)azepan-3- |
| | | (trifluoromethyl)phenyl)cyclopropane- | | | yl)cyclopropane-1-carboxamide |
| | | 1-carboxamide | | | |
| 6B* | 6 | 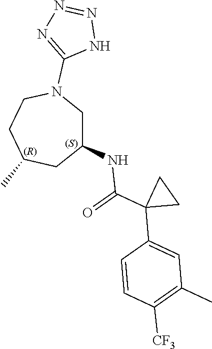 | 54B* | TA | 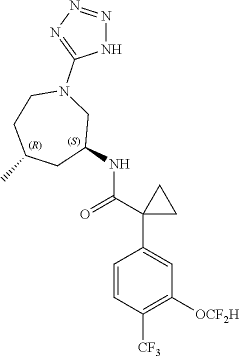 |
| | | N-((3S,5R)-5-methyl-1-(1H- | | | 1-(3-(difluoromethoxy)-4- |
| | | tetrazol-5-yl)azepan-3-yl)-1-(3- | | | (trifluoromethyl)phenyl)-N-((3S,5R)-5- |
| | | methyl-4- | | | methyl-1-(1H-tetrazol-5-yl)azepan-3- |
| | | (trifluoromethyl)phenyl)cyclopropane- | | | yl)cyclopropane-1-carboxamide |
| | | 1-carboxamide | | | |
| 6C* | 6 | 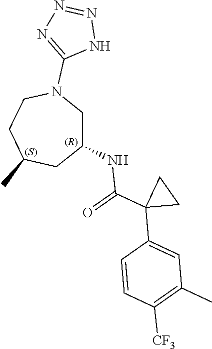 | 54C* | TA | 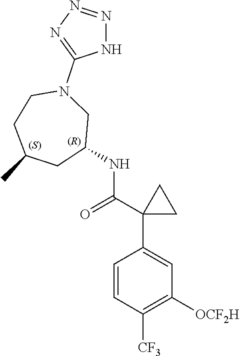 |
| | | N-((3R,5S)-5-methyl-1-(1H- | | | 1-(3-(difluoromethoxy)-4- |
| | | tetrazol-5-yl)azepan-3-yl)-1-(3- | | | (trifluoromethyl)phenyl)-N-((3R,5S)-5- |
| | | methyl-4- | | | methyl-1-(1H-tetrazol-5-yl)azepan-3- |
| | | (trifluoromethyl)phenyl)cycloprop | | | yl)cyclopropane-1-carboxamide |
| | | ane-1-carboxamide | | | |
| 6D* | 6 | 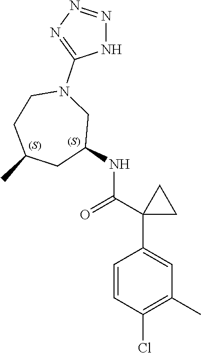 | 54D* | TA | 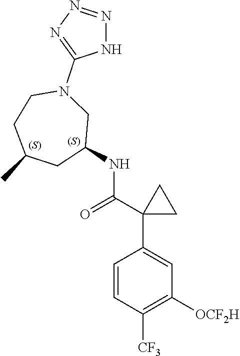 |
| | | N-((3S,5S)-5-methyl-1-(1H- | | | 1-(3-(difluoromethoxy)-4- |
| | | tetrazol-5-yl)azepan-3-yl)-1-(3- | | | (trifluoromethyl)phenyl)-N-((3S,5S)-5- |
| | | methyl-4- | | | methyl-1-(1H-tetrazol-5-yl)azepan-3- |
| | | (trifluoromethyl)phenyl)cycloprop | | | yl)cyclopropane-1-carboxamide |
| | | ane-1-carboxamide | | | |
| 7A* | 7 | 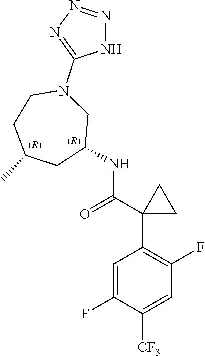 | Rac-55 | TA | 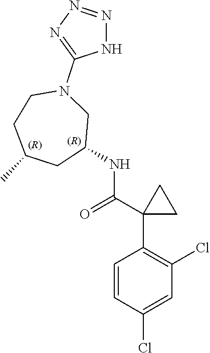 |
| | | 1-(2,5-difluoro-4- | | | 1-(3-(difluoromethoxy)-4- |
| | | (trifluoromethyl)phenyl)-N- | | | (trifluoromethyl)phenyl)-N-(5-methyl-1- |
| | | ((3R,5R)-5-methyl-1-(1H-tetrazol- | | | (1H-tetrazol-5-yl)azepan-3- |
| | | 5-yl)azepan-3-yl)cyclopropane-1- | | | yl)cyclopropane-1-carboxamide |
| | | carboxamide | | | |
| 7B* | 7 | 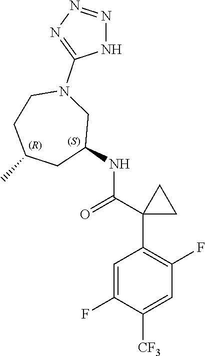 | 55A* | TA | 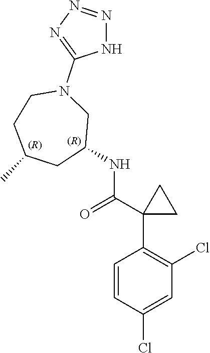 |
| | | 1-(2,5-difluoro-4- | | | 1-(2,4-dichlorophenyl)-N-((3R,5R)-5- |
| | | (trifluoromethyl)phenyl)-N- | | | methyl-1-(1H-tetrazol-5-yl)azepan-3- |
| | | ((3S,5R)-5-methyl-1-(1H-tetrazol- | | | yl)cyclopropane-1-carboxamide |
| | | 5-yl)azepan-3-yl)cyclopropane-1- | | | |
| | | carboxamide | | | |
| 7C* | 7 | 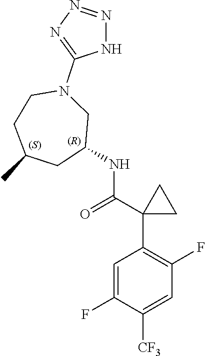 | 55B* | TA | 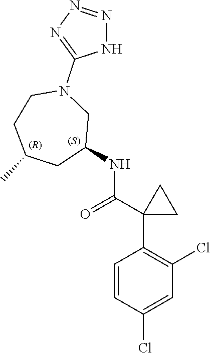 |
| | | 1-(2,5-difluoro-4- | | | 1-(2,4-dichlorophenyl)-N-((3S,5R)-5- |
| | | (trifluoromethyl)phenyl)-N- | | | methyl-1-(1H-tetrazol-5-yl)azepan-3- |
| | | ((3R,5S)-5-methyl-1-(1H-tetrazol- | | | |
| | | 5-yl)azepan-3-yl)cyclopropane-1- | | | |
| | | carboxamide | | | |
| 7D* | 7 | 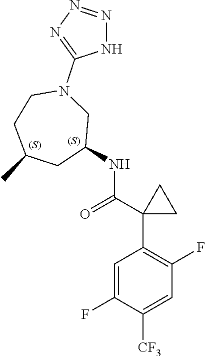 | 55C* | TA | 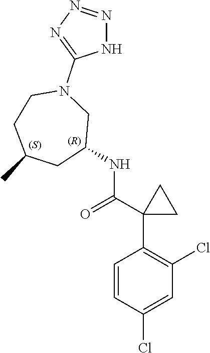 |
| | | 1-(2,5-difluoro-4- | | | 1-(2,4-dichlorophenyl)-N-((3R,5S)-5- |
| | | (trifluoromethyl)phenyl)-N- | | | methyl-1-(1H-tetrazol-5-yl)azepan-3- |
| | | ((3S,5S)-5-methyl-1-(1H-tetrazol- | | | |
| | | 5-yl)azepan-3-yl)cyclopropane-1- | | | |
| | | carboxamide | | | |
| 8A* | 8 | 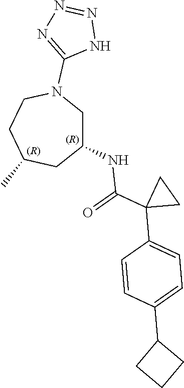 | 55D* | TA | 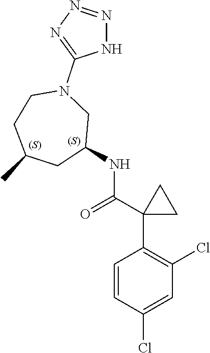 |
| | | 1-(4-cyclobutylphenyl)-N- | | | 1-(2,4-dichlorophenyl)-N-((3S,5S)-5- |
| | | ((3R,5R)-5-methyl-1-(1H-tetrazol- | | | methyl-1-(1H-tetrazol-5-yl)azepan-3- |
| | | 5-yl)azepan-3-yl)cyclopropane-1- | | | |
| | | carboxamide | | | |
| 8B* | 8 | 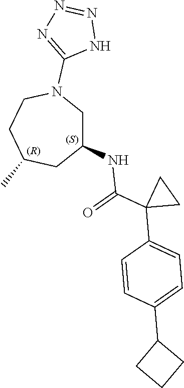 | Rac-56 | TA | 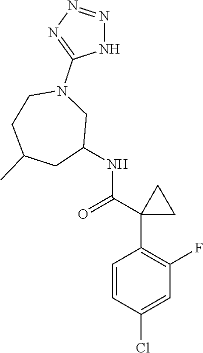 [SMILES: CC1CCN(c2nnn[nH]2)CC(NC(=O)C2(c3ccc(Cl)cc3F)CC2)C1] |
| | | 1-(4-cyclobutylphenyl)-N- | | | 1-(4-chloro-2-fluorophenyl)-N-(5- |
| | | ((3S,5R)-5-methyl-1-(1H-tetrazol- | | | methyl-1-(1H-tetrazol-5-yl)azepan-3- |
| | | 5-yl)azepan-3-yl)cyclopropane-1- | | | yl)cyclopropane-1-carboxamide |
| | | carboxamide | | | |
| 8C* | 8 | 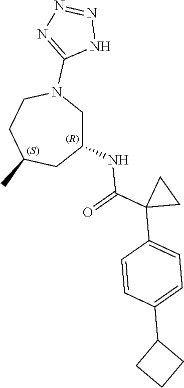 | 56A* | TA | 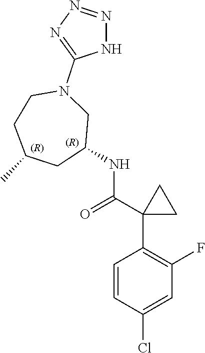 |
| | | 1-(4-cyclobutylphenyl)-N- | | | 1-(4-chloro-2-fluorophenyl)-N- |
| | | ((3R,5S)-5-methyl-1-(1H-tetrazol- | | | ((3R,5R)-5-methyl-1-(1H-tetrazol-5- |
| | | 5-yl)azepan-3-yl)cyclopropane-1- | | | yl)azepan-3-yl)cyclopropane-1- |
| | | carboxamide | | | carboxamide |
| 8D* | 8 | 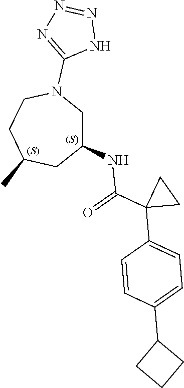 | 56B* | TA | 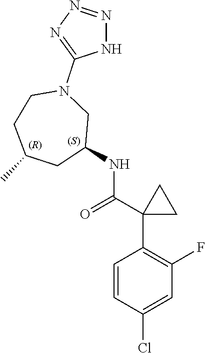 |
| | | 1-(4-cyclobutylphenyl)-N- | | | 1-(4-chloro-2-fluorophenyl)-N-((3S,5R)- |
| | | ((3S,5S)-5-methyl-1-(1H-tetrazol- | | | 5-methyl-1-(1H-tetrazol-5-yl)azepan-3- |
| | | 5-yl)azepan-3-yl)cyclopropane-1- | | | yl)cyclopropane-1-carboxamide |
| | | carboxamide | | | |
| 9′* | 9 | 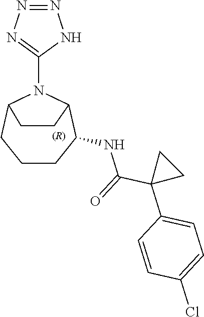 | 56C* | TA | 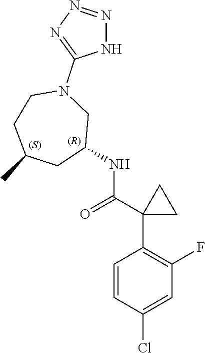 |
| | | N-((2R)-9-(1H-tetrazol-5-yl)-9- | | | 1-(4-chloro-2-fluorophenyl)-N-((3R,5S)- |
| | | azabicyclo[4.2.1]nonan-2-yl)-1-(4- | | | 5-methyl-1-(1H-tetrazol-5-yl)azepan-3- |
| | | chlorophenyl)cyclopropane-1- | | | yl)cyclopropane-1-carboxamide |
| | | carboxamide | | | |
| 9″* | 9 | 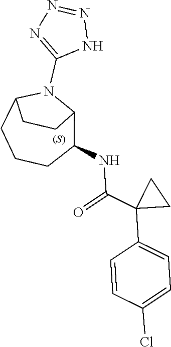 | 56D* | TA | 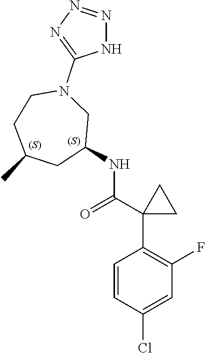 |
| | | N-((2S)-9-(1H-tetrazol-5-yl)-9- | | | 1-(4-chloro-2-fluorophenyl)-N-((3S,5S)- |
| | | azabicyclo[4.2.1]nonan-2-yl)-1-(4- | | | 5-methyl-1-(1H-tetrazol-5-yl)azepan-3- |
| | | chlorophenyl)cyclopropane-1- | | | yl)cyclopropane-1-carboxamide |
| | | carboxamide | | | |
| 9A* | 9 | 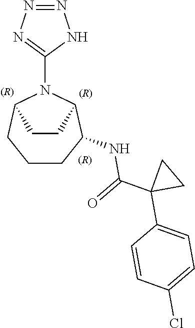 | Rac-57 | TA | 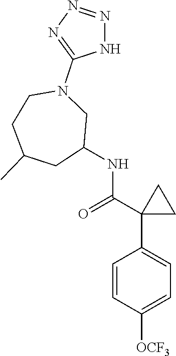 [SMILES: CC1CCN(c2nnn[nH]2)CC(NC(=O)C2(c3ccc(OC(F)(F)F)cc3)CC2)C1] |
| | | N-((1R,2R,6R)-9-(1H-tetrazol-5- | | | N-(5-methyl-1-(1H-tetrazol-5-yl)azepan- |
| | | yl)-9-azabicyclo[4.2.1]nonan-2- | | | 3-yl)-1-(4- |
| | | yl)-1-(4- | | | (trifluoromethoxy)phenyl)cyclopropane- |
| | | chlorophenyl)cyclopropane-1- | | | 1-carboxamide |
| | | carboxamide | | | |
| 9B* | 9 | 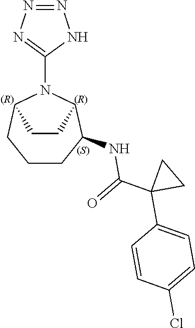 | 57A* | TA | 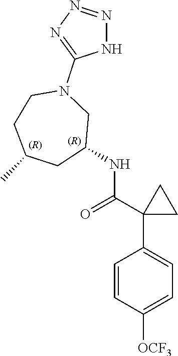 |
| | | N-((1R,2S,6R)-9-(1H-tetrazol-5- | | | N-((3R,5R)-5-methyl-1-(1H-tetrazol-5- |
| | | yl)-9-azabicyclo[4.2.1]nonan-2- | | | yl)azepan-3-yl)-1-(4- |
| | | yl)-1-(4- | | | (trifluoromethoxy)phenyl)cyclopropane- |
| | | chlorophenyl)cyclopropane-1- | | | 1-carboxamide |
| | | carboxamide | | | |
| 9C* | 9 | 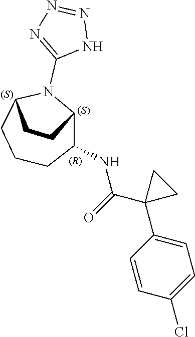 | 57B* | TA | 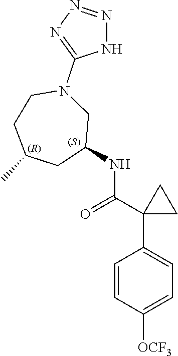 |
| | | N-((1S,2R,6S)-9-(1H-tetrazol-5- | | | N-((3S,5R)-5-methyl-1-(1H-tetrazol-5- |
| | | yl)-9-azabicyclo[4.2.1]nonan-2- | | | yl)azepan-3-yl)-1-(4- |
| | | yl)-1-(4- | | | (trifluoromethoxy)phenyl)cyclopropane- |
| | | chlorophenyl)cyclopropane-1- | | | 1-carboxamide |
| | | carboxamide | | | |
| 9D* | 9 | 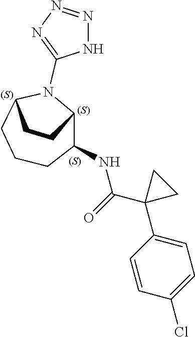 | 57C* | TA | 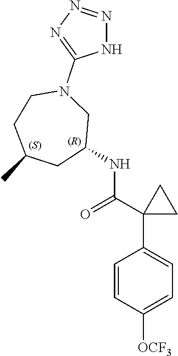 |
| | | N-((1S,2S,6S)-9-(1H-tetrazol-5- | | | N-((3R,5S)-5-methyl-1-(1H-tetrazol-5- |
| | | yl)-9-azabicyclo[4.2.1]nonan-2- | | | yl)azepan-3-yl)-1-(4- |
| | | yl)-1-(4- | | | (trifluoromethoxy)phenyl)cyclopropane- |
| | | chlorophenyl)cyclopropane-1- | | | 1-carboxamide |
| | | carboxamide | | | |
| 10A | 10 | 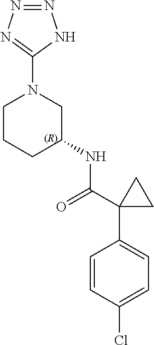 | 57D* | TA | 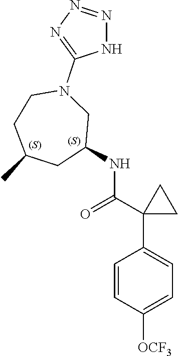 |
| | | (R)-N-(1-(1H-tetrazol-5- | | | N-((3S,5S)-5-methyl-1-(1H-tetrazol-5- |
| | | yl)piperidin-3-yl)-1-(4- | | | yl)azepan-3-yl)-1-(4- |
| | | chlorophenyl)cyclopropane-1- | | | (trifluoromethoxy)phenyl)cyclopropane- |
| | | carboxamide | | | 1-carboxamide |
| 10B | 10 | 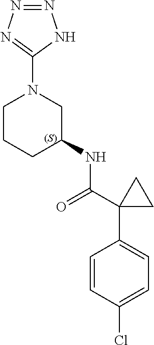 | Rac-58 | TA | 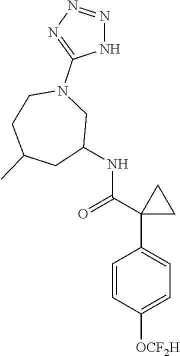 |
| | | (S)-N-(1-(1H-tetrazol-5- | | | 1-(4-(difluoromethoxy)phenyl)-N-(5- |
| | | yl)piperidin-3-yl)-1-(4- | | | methyl-1-(1H-tetrazol-5-yl)azepan-3- |
| | | chlorophenyl)cyclopropane-1- | | | yl)cyclopropane-1-carboxamide |
| | | carboxamide | | | |
| 11A* | 11 | 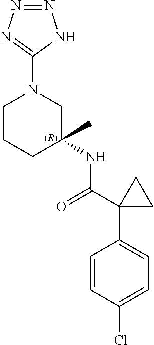 | 58A* | TA | 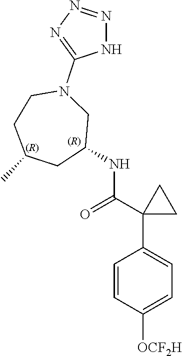 |
| | | (R)-1-(4-chlorophenyl)-N-(3- | | | 1-(4-(difluoromethoxy)phenyl)-N- |
| | | methyl-1-(1H-tetrazol-5- | | | ((3R,5R)-5-methyl-1-(1H-tetrazol-5- |
| | | yl)piperidin-3-yl)cyclopropane-1- | | | yl)azepan-3-yl)cyclopropane-1- |
| | | carboxamide | | | carboxamide |
| 11B* | 11 | 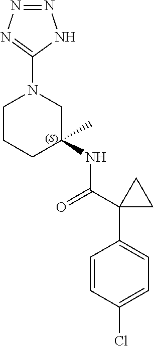 | 58B* | TA | 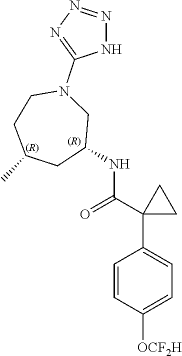 |
| | | (S)-1-(4-chlorophenyl)-N-(3- | | | 1-(4-(difluoromethoxy)phenyl)-N- |
| | | methyl-1-(1H-tetrazol-5- | | | ((3S,5R)-5-methyl-1-(1H-tetrazol-5- |
| | | yl)piperidin-3-yl)cyclopropane-1- | | | yl)azepan-3-yl)cyclopropane-1- |
| | | carboxamide | | | carboxamide |
| 12A* | 12 | 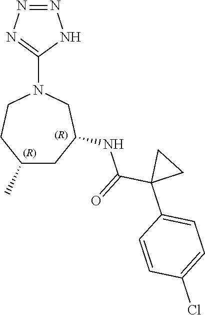 | 58C* | TA | 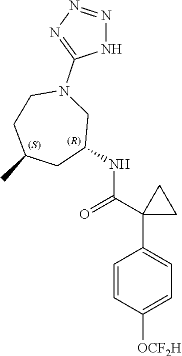 |
| | | 1-(4-chlorophenyl)-N-((3R,5R)-5- | | | 1-(4-(difluoromethoxy)phenyl)-N- |
| | | methyl-1-(1H-tetrazol-5-yl)azepan- | | | ((3R,5S)-5-methyl-1-(1H-tetrazol-5- |
| | | 3-yl)cyclopropane-1-carboxamide | | | yl)azepan-3-yl)cyclopropane-1- |
| | | | | | carboxamide |
| 12B* | 12 | 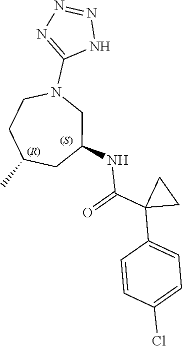 | 58D* | TA | 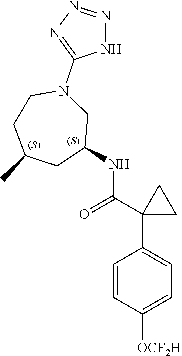 |
| | | 1-(4-chlorophenyl)-N-((3S,5R)-5- | | | 1-(4-(difluoromethoxy)phenyl)-N- |
| | | methyl-1-(1H-tetrazol-5-yl)azepan- | | | ((3S,5S)-5-methyl-1-(1H-tetrazol-5- |
| | | 3-yl)cyclopropane-1-carboxamide | | | yl)azepan-3-yl)cyclopropane-1- |
| | | | | | carboxamide |
| 12C* | 12 | 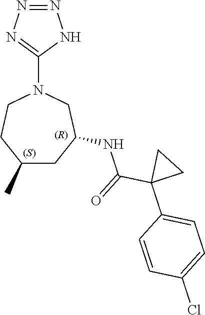 | Rac-59 | TA | 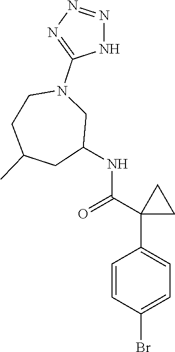 [SMILES: CC1CCN(c2nnn[nH]2)CC(NC(=O)C2(c3ccc(Br)cc3)CC2)C1] |
| | | 1-(4-chlorophenyl)-N-((3R,5S)-5- | | | 1-(4-bromophenyl)-N-(5-methyl-1-(1H- |
| | | methyl-1-(1H-tetrazol-5-yl)azepan- | | | tetrazol-5-yl)azepan-3-yl)cyclopropane- |
| | | 3-yl)cyclopropane-1-carboxamide | | | 1-carboxamide |
| 12D* | 12 | 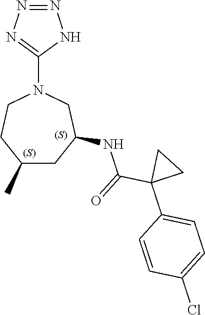 | 59A* | TA | 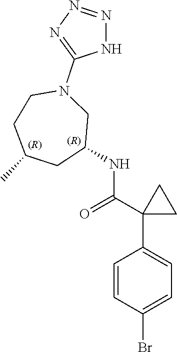 |
| | | 1-(4-chlorophenyl)-N-((3S,5S)-5- | | | 1-(4-bromophenyl)-N-((3R,5R)-5- |
| | | methyl-1-(1H-tetrazol-5-yl)azepan- | | | methyl-1-(1H-tetrazol-5-yl)azepan-3- |
| | | 3-yl)cyclopropane-1-carboxamide | | | yl)cyclopropane-1-carboxamide |
| 13A* | 13 | 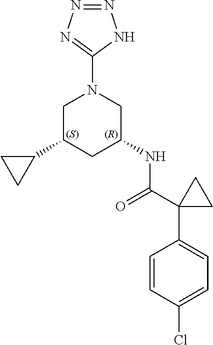 | 59B* | TA | 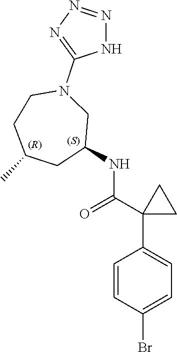 |
| | | 1-(4-chlorophenyl)-N-((3R,5S)-5- | | | 1-(4-bromophenyl)-N-((3S,5R)-5- |
| | | cyclopropyl-1-(1H-tetrazol-5- | | | methyl-1-(1H-tetrazol-5-yl)azepan-3- |
| | | yl)piperidin-3-yl)cyclopropane-1- | | | yl)cyclopropane-1-carboxamide |
| | | carboxamide | | | |
| 13B* | 13 | 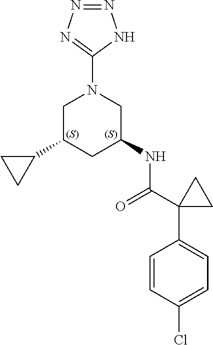 | 59C* | TA | 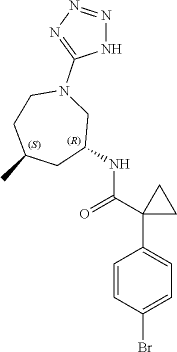 |
| | | 1-(4-chlorophenyl)-N-((3S,5S)-5- | | | 1-(4-bromophenyl)-N-((3R,5S)-5- |
| | | cyclopropyl-1-(1H-tetrazol-5- | | | methyl-1-(1H-tetrazol-5-yl)azepan-3- |
| | | yl)piperidin-3-yl)cyclopropane-1- | | | yl)cyclopropane-1-carboxamide |
| | | carboxamide | | | |
| 13C* | 13 | 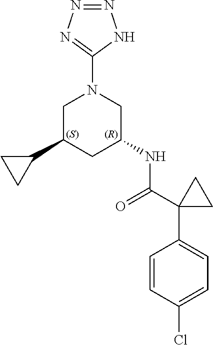 | 59D* | TA | 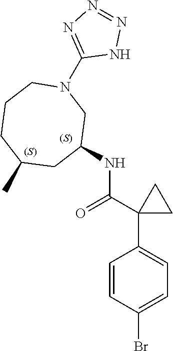 |
| | | 1-(4-chlorophenyl)-N-((3R,5S)-5- | | | 1-(4-bromophenyl)-N-((3S,5S)-5- |
| | | cyclopropyl-1-(1H-tetrazol-5- | | | methyl-1-(1H-tetrazol-5-yl)azepan-3- |
| | | yl)piperidin-3-yl)cyclopropane-1- | | | yl)cyclopropane-1-carboxamide |
| | | carboxamide | | | |
| 13D* | 13 | 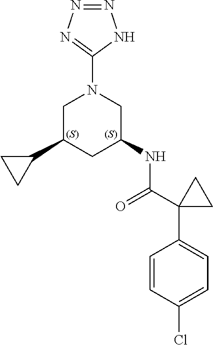 | Rac-60 | TA | 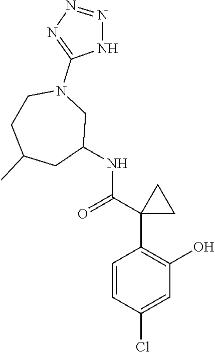 [SMILES: CC1CCN(c2nnn[nH]2)CC(NC(=O)C2(c3ccc(Cl)cc3O)CC2)C1] |
| | | 1-(4-chlorophenyl)-N-((3S,5S)-5- | | | 1-(4-chloro-2-hydroxyphenyl)-N-(5- |
| | | cyclopropyl-1-(1H-tetrazol-5- | | | methyl-1-(1H-tetrazol-5-yl)azepan-3- |
| | | yl)piperidin-3-yl)cyclopropane-1- | | | yl)cyclopropane-1-carboxamide |
| | | carboxamide | | | |
| 14A* | 14 | 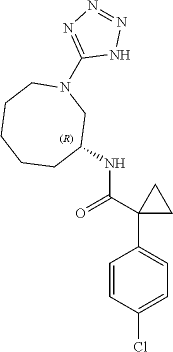 | 60A* | TA | 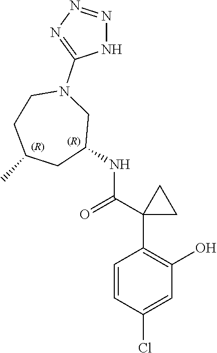 |
| | | (R)-N-(1-(1H-tetrazol-5-yl)azocan- | | | 1-(4-chloro-2-hydroxyphenyl)-N- |
| | | 3-yl)-1-(4- | | | ((3R,5R)-5-methyl-1-(1H-tetrazol-5- |
| | | chlorophenyl)cyclopropane-1- | | | yl)azepan-3-yl)cyclopropane-1- |
| | | carboxamide | | | carboxamide |
| 14B* | 14 | 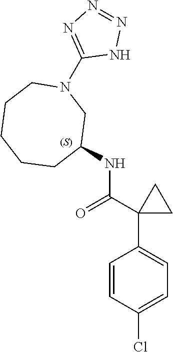 | 60B* | TA | 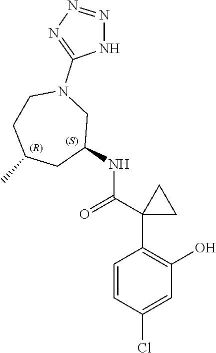 |
| | | (S)-N-(1-(1H-tetrazol-5-yl)azocan- | | | 1-(4-chloro-2-hydroxyphenyl)-N- |
| | | 3-yl)-1-(4- | | | ((3S,5R)-5-methyl-1-(1H-tetrazol-5- |
| | | chlorophenyl)cyclopropane-1- | | | yl)azepan-3-yl)cyclopropane-1- |
| | | carboxamide | | | carboxamide |
| 15A | 15 | 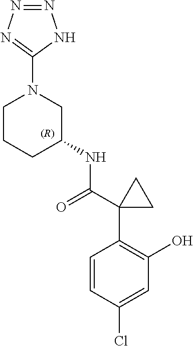 | 60C* | TA | 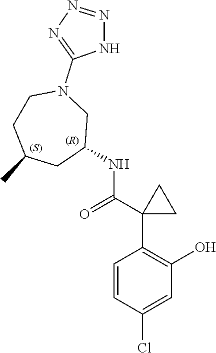 |
| | | (R)-N-(1-(1H-tetrazol-5- | | | 1-(4-chloro-2-hydroxyphenyl)-N- |
| | | yl)piperidin-3-yl)-1-(4-chloro-2- | | | ((3R,5S)-5-methyl-1-(1H-tetrazol-5- |
| | | hydroxyphenyl)cyclopropane-1- | | | yl)azepan-3-yl)cyclopropane-1- |
| | | carboxamide | | | carboxamide |
| 15B | 15 | 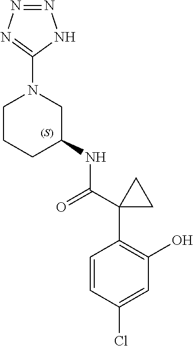 | 60D* | TA | 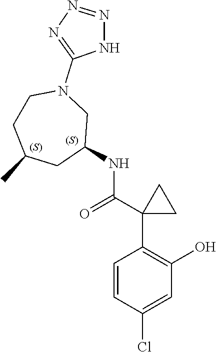 |
| | | (S)-N-(1-(1H-tetrazol-5- | | | 1-(4-chloro-2-hydroxyphenyl)-N- |
| | | yl)piperidin-3-yl)-1-(4-chloro-2- | | | ((3S,5S)-5-methyl-1-(1H-tetrazol-5- |
| | | hydroxyphenyl)cyclopropane-1- | | | yl)azepan-3-yl)cyclopropane-1- |
| | | carboxamide | | | carboxamide |
| 15A- OMe | 15 | 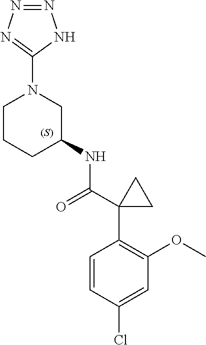 | Rac-61 | TA | 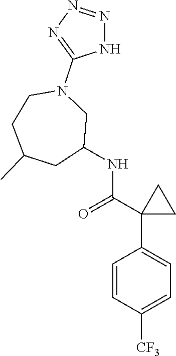 [SMILES: CC1CCN(c2nnn[nH]2)CC(NC(=O)C2(c3ccc(C(F)(F)F)cc3)CC2)C1] |
| | | (R)-N-(1-(1H-tetrazol-5- | | | N-(5-methyl-1-(1H-tetrazol-5-yl)azepan- |
| | | yl)piperidin-3-yl)-1-(4-chloro-2- | | | 3-yl)-1-(4- |
| | | methoxyphenyl)cyclopropane-1- | | | (trifluoromethyl)phenyl)cyclopropane-1- |
| | | carboxamide | | | carboxamide |
| 15B- OMe | 15 | 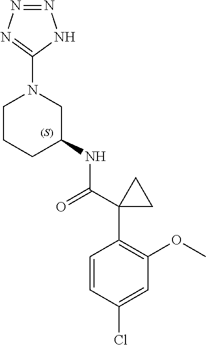 | 61A* | TA | 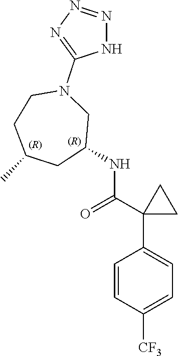 |
| | | (S)-N-(1-(1H-tetrazol-5- | | | N-((3R,5R)-5-methyl-1-(1H-tetrazol-5- |
| | | yl)piperidin-3-yl)-1-(4-chloro-2- | | | yl)azepan-3-yl)-1-(4- |
| | | methoxyphenyl)cyclopropane-1- | | | (trifluoromethyl)phenyl)cyclopropane-1- |
| | | carboxamide | | | carboxamide |
| 16A | 16 | 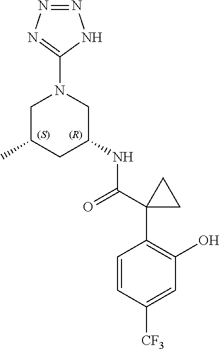 | 61B* | TA | 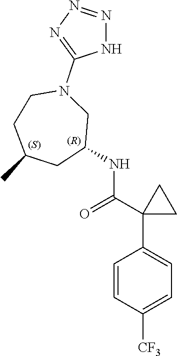 |
| | | 1-(2-hydroxy-4- | | | N-((3R,5S)-5-methyl-1-(1H-tetrazol-5- |
| | | (trifluoromethyl)phenyl)-N- | | | yl)azepan-3-yl)-1-(4- |
| | | ((3R,5S)-5-methyl-1-(1H-tetrazol- | | | (trifluoromethyl)phenyl)cyclopropane-1- |
| | | 5-yl)piperidin-3-yl)cyclopropane- | | | carboxamide |
| | | 1-carboxamide | | | |
| 16B | 16 | 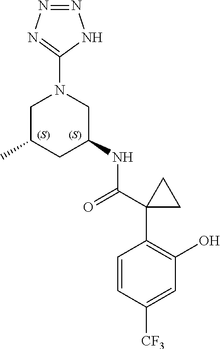 | 61C* | TA | 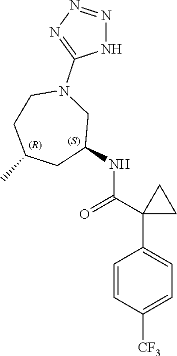 |
| | | 1-(2-hydroxy-4- | | | N-((3S,5R)-5-methyl-1-(1H-tetrazol-5- |
| | | (trifluoromethyl)phenyl)-N- | | | yl)azepan-3-yl)-1-(4- |
| | | ((3S,5S)-5-methyl-1-(1H-tetrazol- | | | (trifluoromethyl)phenyl)cyclopropane-1- |
| | | 5-yl)piperidin-3-yl)cyclopropane- | | | carboxamide |
| | | 1-carboxamide | | | |
| 16C | 16 | 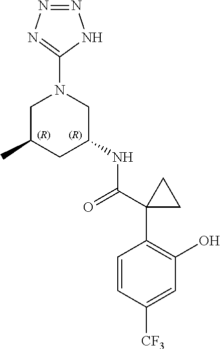 | 61D* | TA | 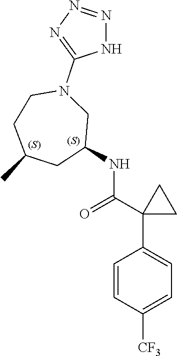 |
| | | 1-(2-hydroxy-4- | | | N-((3S,5S)-5-methyl-1-(1H-tetrazol-5- |
| | | (trifluoromethyl)phenyl)-N- | | | yl)azepan-3-yl)-1-(4- |
| | | ((3R,5R)-5-methyl-1-(1H-tetrazol- | | | (trifluoromethyl)phenyl)cyclopropane-1- |
| | | 5-yl)piperidin-3-yl)cyclopropane- | | | carboxamide |
| | | 1-carboxamide | | | |
| 16D | 16 | 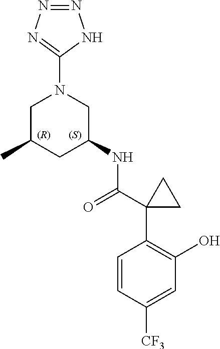 | Rac-62 | TA | 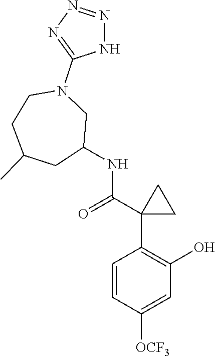 [SMILES: CC1CCN(c2nnn[nH]2)CC(NC(=O)C2(c3ccc(OC(F)(F)F)cc3O)CC2)C1] |
| | | 1-(2-hydroxy-4- | | | 1-(2-hydroxy-4- |
| | | (trifluoromethyl)phenyl)-N- | | | (trifluoromethoxy)phenyl)-N-(5-methyl- |
| | | ((3S,5R)-5-methyl-1-(1H-tetrazol- | | | 1-(1H-tetrazol-5-yl)azepan-3- |
| | | 5-yl)piperidin-3-yl)cyclopropane- | | | yl)cyclopropane-1-carboxamide |
| | | 1-carboxamide | | | |
| 16A- OMe | 16 | 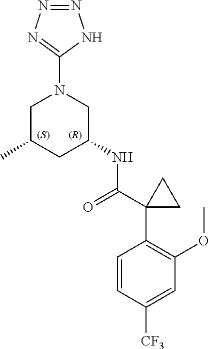 | 62A* | TA | 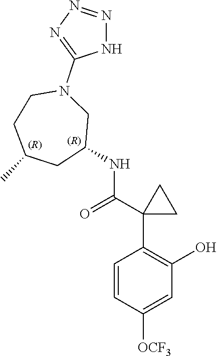 |
| | | 1-(2-methoxy-4- | | | 1-(2-hydroxy-4- |
| | | (trifluoromethyl)phenyl)-N- | | | (trifluoromethoxy)phenyl)-N-((3R,5R)- |
| | | ((3R,5S)-5-methyl-1-(1H-tetrazol- | | | 5-methyl-1-(1H-tetrazol-5-yl)azepan-3- |
| | | 5-yl)piperidin-3-yl)cyclopropane- | | | yl)cyclopropane-1-carboxamide |
| | | 1-carboxamide | | | |
| 16B- OMe | 16 | 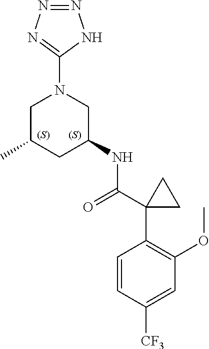 | 62B* | TA | 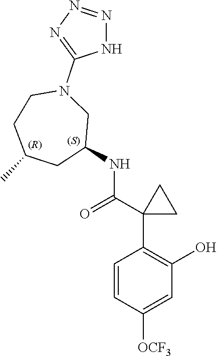 |
| | | 1-(2-methoxy-4- | | | 1-(2-hydroxy-4- |
| | | (trifluoromethyl)phenyl)-N- | | | (trifluoromethoxy)phenyl)-N-((3S,5R)- |
| | | ((3S,5S)-5-methyl-1-(1H-tetrazol- | | | 5-methyl-1-(1H-tetrazol-5-yl)azepan-3- |
| | | 5-yl)piperidin-3-yl)cyclopropane- | | | yl)cyclopropane-1-carboxamide |
| | | 1-carboxamide | | | |
| 16C- OMe | 16 | 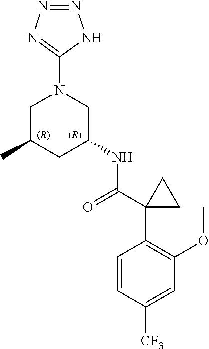 | 62C* | TA | 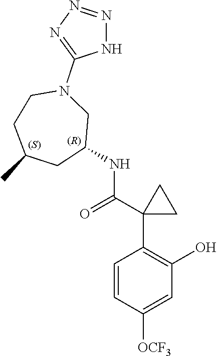 |
| | | 1-(2-methoxy-4- | | | 1-(2-hydroxy-4- |
| | | (trifluoromethyl)phenyl)-N- | | | (trifluoromethoxy)phenyl)-N-((3R,5S)- |
| | | ((3R,5R)-5-methyl-1-(1H-tetrazol- | | | 5-methyl-1-(1H-tetrazol-5-yl)azepan-3- |
| | | 5-yl)piperidin-3-yl)cyclopropane- | | | yl)cyclopropane-1-carboxamide |
| | | 1-carboxamide | | | |
| 16D- OMe | 16 | 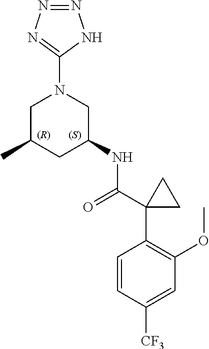 | 62D* | TA | 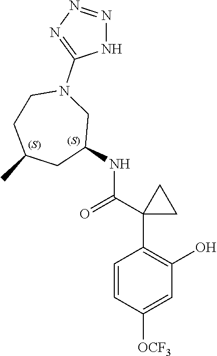 |
| | | 1-(2-methoxy-4- | | | 1-(2-hydroxy-4- |
| | | (trifluoromethyl)phenyl)-N- | | | (trifluoromethoxy)phenyl)-N-((3S,5R)- |
| | | ((3S,5R)-5-methyl-1-(1H-tetrazol- | | | 5-methyl-1-(1H-tetrazol-5-yl)azepan-3- |
| | | 5-yl)piperidin-3-yl)cyclopropane- | | | yl)cyclopropane-1-carboxamide |
| | | 1-carboxamide | | | |
| 17A | 17 | 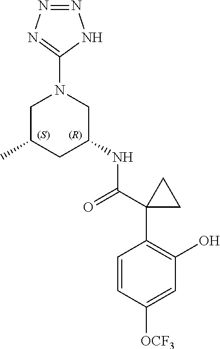 | Rac-63 | TA | 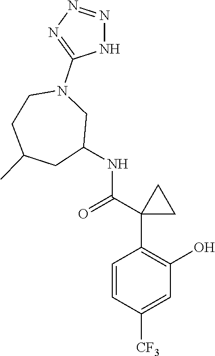 [SMILES: CC1CCN(c2nnn[nH]2)CC(NC(=O)C2(c3ccc(C(F)(F)F)cc3O)CC2)C1] |
| | | 1-(2-hydroxy-4- | | | 1-(2-hydroxy-4- |
| | | (trifluoromethoxy)phenyl)-N- | | | (trifluoromethyl)phenyl)-N-(5-methyl-1- |
| | | ((3R,5S)-5-methyl-1-(1H-tetrazol- | | | (1H-tetrazol-5-yl)azepan-3- |
| | | 5-yl)piperidin-3-yl)cyclopropane- | | | yl)cyclopropane-1-carboxamide |
| | | 1-carboxamide | | | |
| 17B | 17 | 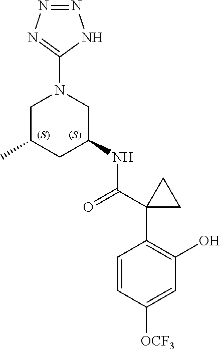 | 63A* | TA | 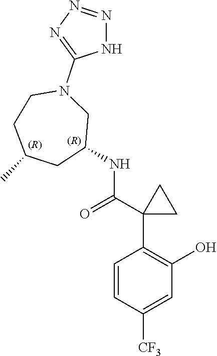 |
| | | 1-(2-hydroxy-4- | | | 1-(2-hydroxy-4- |
| | | (trifluoromethoxy)phenyl)-N- | | | (trifluoromethyl)phenyl)-N-((3R,5R)-5- |
| | | ((3S,5S)-5-methyl-1-(1H-tetrazol- | | | methyl-1-(1H-tetrazol-5-yl)azepan-3- |
| | | 5-yl)piperidin-3-yl)cyclopropane- | | | yl)cyclopropane-1-carboxamide |
| | | 1-carboxamide | | | |
| 17C | 17 | 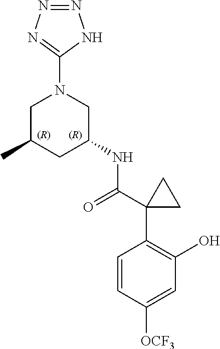 | 63B* | TA | 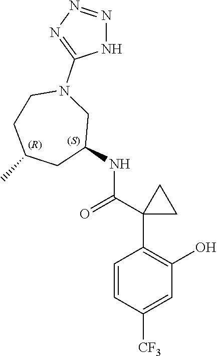 |
| | | 1-(2-hydroxy-4- | | | 1-(2-hydroxy-4- |
| | | (trifluoromethoxy)phenyl)-N- | | | (trifluoromethyl)phenyl)-N-((3S,5R)-5- |
| | | ((3R,5R)-5-methyl-1-(1H-tetrazol- | | | methyl-1-(1H-tetrazol-5-yl)azepan-3- |
| | | 5-yl)piperidin-3-yl)cyclopropane- | | | yl)cyclopropane-1-carboxamide |
| | | 1-carboxamide | | | |
| 17D | 17 | 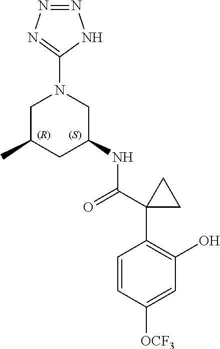 | 63C* | TA | 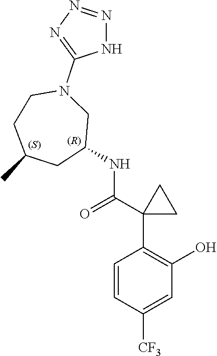 |
| | | 1-(2-hydroxy-4- | | | 1-(2-hydroxy-4- |
| | | (trifluoromethoxy)phenyl)-N- | | | (trifluoromethyl)phenyl)-N-((3R,5S)-5- |
| | | ((3S,5R)-5-methyl-1-(1H-tetrazol- | | | methyl-1-(1H-tetrazol-5-yl)azepan-3- |
| | | 5-yl)piperidin-3-yl)cyclopropane- | | | yl)cyclopropane-1-carboxamide |
| | | 1-carboxamide | | | |
| 17A- OMe | 17 | 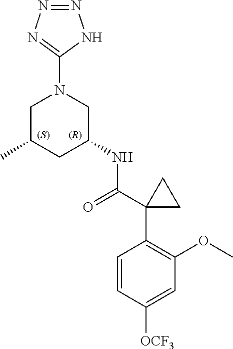 | 63D* | TA | 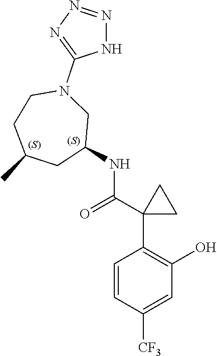 |
| | | 1-(2-methoxy-4- | | | 1-(2-hydroxy-4- |
| | | (trifluoromethoxy)phenyl)-N- | | | (trifluoromethyl)phenyl)-N-((3S,5S)-5- |
| | | ((3R,5S)-5-methyl-1-(1H-tetrazol- | | | methyl-1-(1H-tetrazol-5-yl)azepan-3- |
| | | 5-yl)piperidin-3-yl)cyclopropane- | | | yl)cyclopropane-1-carboxamide |
| | | 1-carboxamide | | | |
| 17B- OMe | 17 | 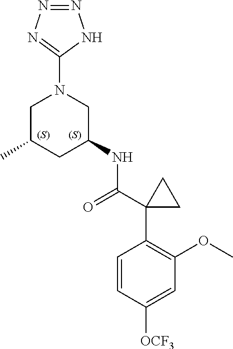 | Rac-64 | TA | 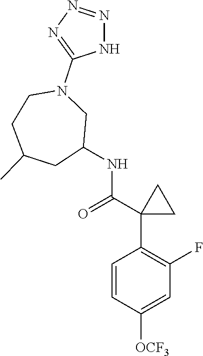 [SMILES: CC1CCN(c2nnn[nH]2)CC(NC(=O)C2(c3ccc(OC(F)(F)F)cc3F)CC2)C1] |
| | | 1-(2-methoxy-4- | | | 1-(2-fluoro-4- |
| | | (trifluoromethoxy)phenyl)-N- | | | (trifluoromethoxy)phenyl)-N-(5-methyl- |
| | | ((3S,5S)-5-methyl-1-(1H-tetrazol- | | | 1-(1H-tetrazol-5-yl)azepan-3- |
| | | 5-yl)piperidin-3-yl)cyclopropane- | | | yl)cyclopropane-1-carboxamide |
| | | 1-carboxamide | | | |
| 17C- OMe | 17 | 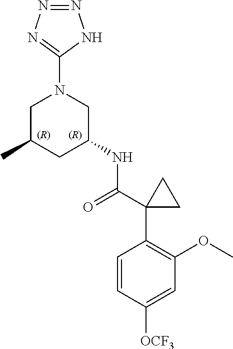 | 64A* | TA | 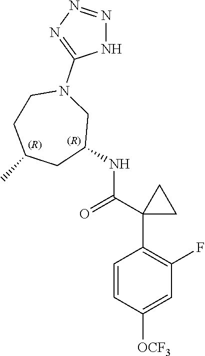 |
| | | 1-(2-methoxy-4- | | | 1-(2-fluoro-4- |
| | | (trifluoromethoxy)phenyl)-N- | | | (trifluoromethoxy)phenyl)-N-((3R,5R)- |
| | | ((3R,5R)-5-methyl-1-(1H-tetrazol- | | | 5-methyl-1-(1H-tetrazol-5-yl)azepan-3- |
| | | 5-yl)piperidin-3-yl)cyclopropane- | | | yl)cyclopropane-1-carboxamide |
| | | 1-carboxamide | | | |
| 17D- OMe | 17 | 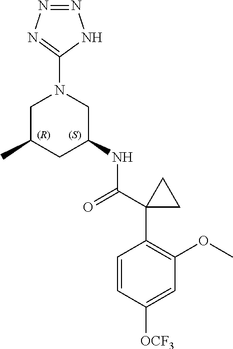 | 64B* | TA | 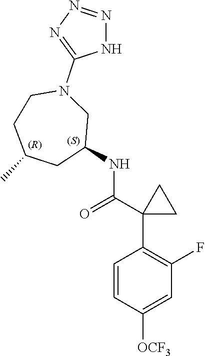 |
| | | 1-(2-methoxy-4- | | | 1-(2-fluoro-4- |
| | | (trifluoromethoxy)phenyl)-N- | | | (trifluoromethoxy)phenyl)-N-((3S,5R)- |
| | | ((3S,5R)-5-methyl-1-(1H-tetrazol- | | | 5-methyl-1-(1H-tetrazol-5-yl)azepan-3- |
| | | 5-yl)piperidin-3-yl)cyclopropane- | | | yl)cyclopropane-1-carboxamide |
| | | 1-carboxamide | | | |
| 18A* | 18 | 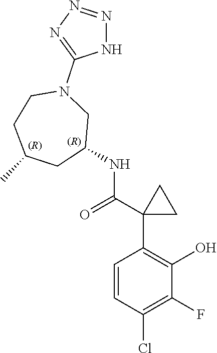 | 64C* | TA | 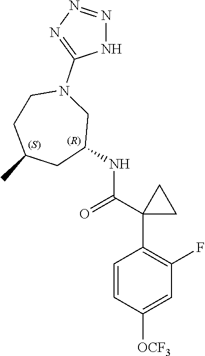 |
| | | 1-(4-chloro-3-fluoro-2- | | | 1-(2-fluoro-4- |
| | | hydroxyphenyl)-N-((3R,5R)-5- | | | (trifluoromethoxy)phenyl)-N-((3R,5S)- |
| | | methyl-1-(1H-tetrazol-5-yl)azepan- | | | 5-methyl-1-(1H-tetrazol-5-yl)azepan-3- |
| | | 3-yl)cyclopropane-1-carboxamide | | | yl)cyclopropane-1-carboxamide |
| 18B* | 18 | 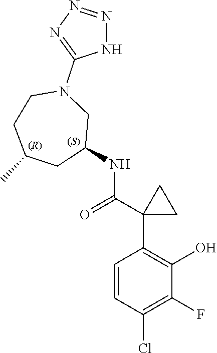 | 64D* | TA | 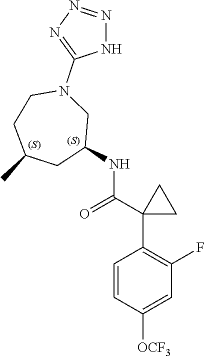 |
| | | 1-(4-chloro-3-fluoro-2- | | | 1-(2-fluoro-4- |
| | | hydroxyphenyl)-N-((3S,5R)-5- | | | (trifluoromethoxy)phenyl)-N-((3S,5S)- |
| | | methyl-1-(1H-tetrazol-5-yl)azepan- | | | 5-methyl-1-(1H-tetrazol-5-yl)azepan-3- |
| | | 3-yl)cyclopropane-1-carboxamide | | | yl)cyclopropane-1-carboxamide |
| 18C* | 18 | 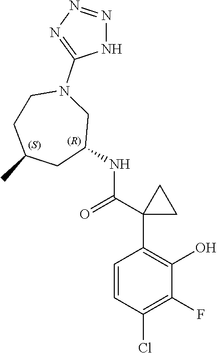 | Rac-65 | TA | 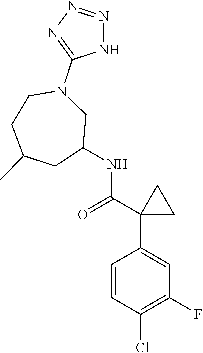 [SMILES: CC1CCN(c2nnn[nH]2)CC(NC(=O)C2(c3ccc(Cl)c(F)c3)CC2)C1] |
| | | 1-(4-chloro-3-fluoro-2- | | | 1-(4-chloro-3-fluorophenyl)-N-(5- |
| | | hydroxyphenyl)-N-((3R,5S)-5- | | | methyl-1-(1H-tetrazol-5-yl)azepan-3- |
| | | methyl-1-(1H-tetrazol-5-yl)azepan- | | | yl)cyclopropane-1-carboxamide |
| | | 3-yl)cyclopropane-1-carboxamide | | | |
| 18D* | 18 | 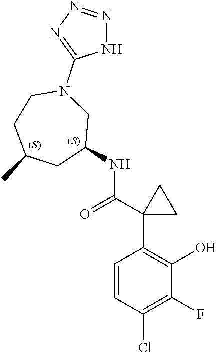 | 65A* | TA | 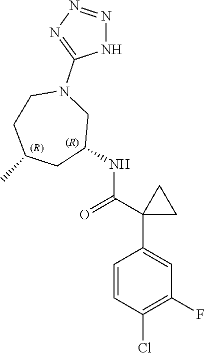 |
| | | 1-(4-chloro-3-fluoro-2- | | | 1-(4-chloro-3-fluorophenyl)-N- |
| | | hydroxyphenyl)-N-((3S,5S)-5- | | | ((3R,5R)-5-methyl-1-(1H-tetrazol-5- |
| | | methyl-1-(1H-tetrazol-5-yl)azepan- | | | yl)azepan-3-yl)cyclopropane-1- |
| | | 3-yl)cyclopropane-1-carboxamide | | | carboxamide |
| 18A- OMe * | 18 | 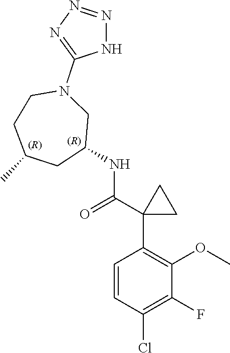 | 65B* | TA | 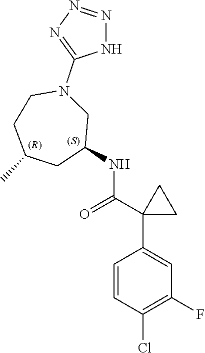 |
| | | 1-(4-chloro-3-fluoro-2- | | | 1-(4-chloro-3-fluorophenyl)-N-((3S,5R)- |
| | | methoxyphenyl)-N-((3R,5R)-5- | | | 5-methyl-1-(1H-tetrazol-5-yl)azepan-3- |
| | | methyl-1-(1H-tetrazol-5-yl)azepan- | | | yl)cyclopropane-1-carboxamide |
| | | 3-yl)cyclopropane-1-carboxamide | | | |
| 18B- OMe* | 18 | 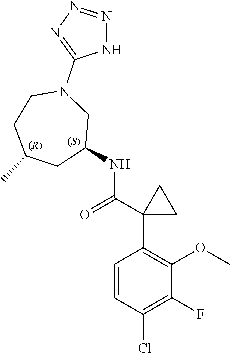 | 65C* | TA | 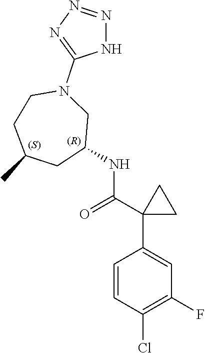 |
| | | 1-(4-chloro-3-fluoro-2- | | | 1-(4-chloro-3-fluorophenyl)-N-((3R,5S)- |
| | | methoxyphenyl)-N-((3S,5R)-5- | | | 5-methyl-1-(1H-tetrazol-5-yl)azepan-3- |
| | | methyl-1-(1H-tetrazol-5-yl)azepan- | | | yl)cyclopropane-1-carboxamide |
| | | 3-yl)cyclopropane-1-carboxamide | | | |
| 18C- OMe* | 18 | 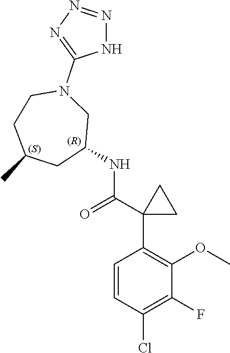 | 65D* | TA | 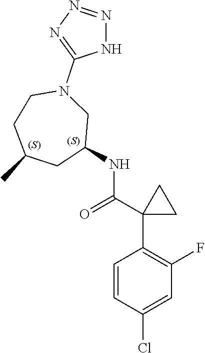 |
| | | 1-(4-chloro-3-fluoro-2- | | | 1-(4-chloro-3-fluorophenyl)-N-((3S,5S)- |
| | | methoxyphenyl)-N-((3R,5S)-5- | | | 5-methyl-1-(1H-tetrazol-5-yl)azepan-3- |
| | | methyl-1-(1H-tetrazol-5-yl)azepan- | | | yl)cyclopropane-1-carboxamide |
| | | 3-yl)cyclopropane-1-carboxamide | | | |
| 18D- OMe* | 18 | 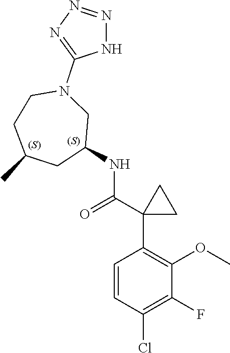 | Rac-66 | TA | 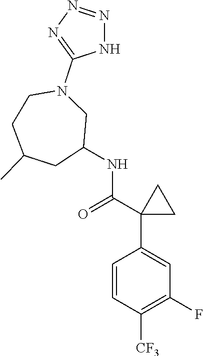 [SMILES: CC1CCN(c2nnn[nH]2)CC(NC(=O)C2(c3ccc(C(F)(F)F)c(F)c3)CC2)C1] |
| | | 1-(4-chloro-3-fluoro-2- | | | 1-(3-fluoro-4-(trifluoromethyl)phenyl)- |
| | | methoxyphenyl)-N-((3S,5S)-5- | | | N-(5-methyl-1-(1H-tetrazol-5-yl)azepan- |
| | | methyl-1-(1H-tetrazol-5-yl)azepan- | | | 3-yl)cyclopropane-1-carboxamide |
| | | 3-yl)cyclopropane-1-carboxamide | | | |
| 19A | 19 | 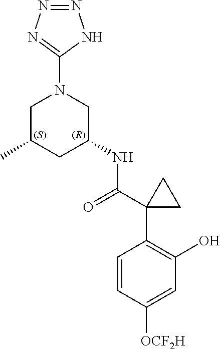 | 66A* | TA | 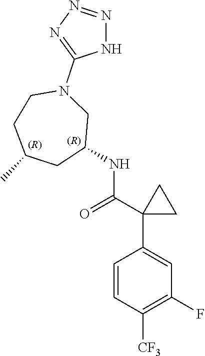 |
| | | 1-(4-(difluoromethoxy)-2- | | | 1-(3-fluoro-4-(trifluoromethyl)phenyl)- |
| | | hydroxyphenyl)-N-((3R,5S)-5- | | | N-((3R,5R)-5-methyl-1-(1H-tetrazol-5- |
| | | methyl-1-(1H-tetrazol-5- | | | yl)azepan-3-yl)cyclopropane-1- |
| | | yl)piperidin-3-yl)cyclopropane-1- | | | carboxamide |
| | | carboxamide | | | |
| 19B | 19 | 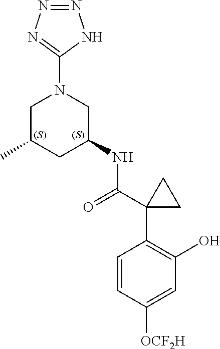 | 66B* | TA | 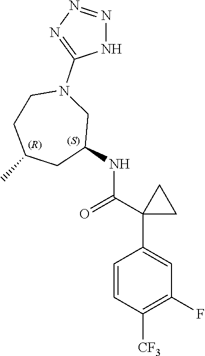 |
| | | 1-(4-(difluoromethoxy)-2- | | | 1-(3-fluoro-4-(trifluoromethyl)phenyl)- |
| | | hydroxyphenyl)-N-((3S,5S)-5- | | | N-((3S,5R)-5-methyl-1-(1H-tetrazol-5- |
| | | methyl-1-(1H-tetrazol-5- | | | yl)azepan-3-yl)cyclopropane-1- |
| | | yl)piperidin-3-yl)cyclopropane-1- | | | carboxamide |
| | | carboxamide | | | |
| 19C | 19 | 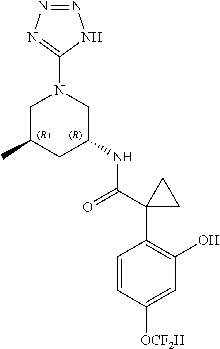 | 66C* | TA | 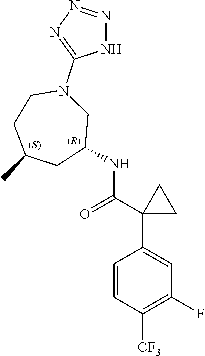 |
| | | 1-(4-(difluoromethoxy)-2- | | | 1-(3-fluoro-4-(trifluoromethyl)phenyl)- |
| | | hydroxyphenyl)-N-((3R,5R)-5- | | | N-((3R,5S)-5-methyl-1-(1H-tetrazol-5- |
| | | methyl-1-(1H-tetrazol-5- | | | yl)azepan-3-yl)cyclopropane-1- |
| | | yl)piperidin-3-yl)cyclopropane-1- | | | carboxamide |
| | | carboxamide | | | |
| 19D | 19 | 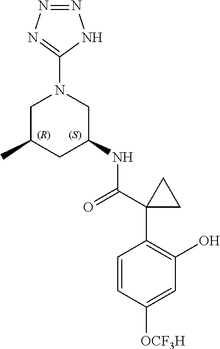 | 66D* | TA | 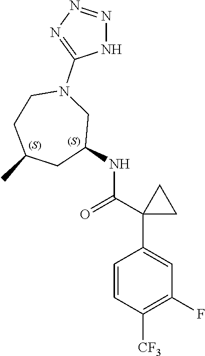 |
| | | 1-(4-(difluoromethoxy)-2- | | | 1-(3-fluoro-4-(trifluoromethyl)phenyl)- |
| | | hydroxyphenyl)-N-((3S,5R)-5- | | | N-((3S,5S)-5-methyl-1-(1H-tetrazol-5- |
| | | methyl-1-(1H-tetrazol-5- | | | yl)azepan-3-yl)cyclopropane-1- |
| | | yl)piperidin-3-yl)cyclopropane-1- | | | carboxamide |
| | | carboxamide | | | |
| 19A- OMe | 19 | 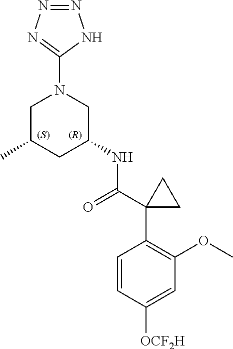 | Rac-67 | TA | 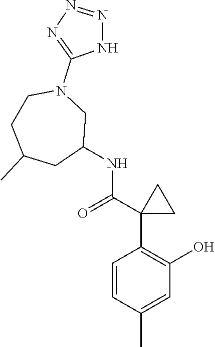 [SMILES: Cc1ccc(C2(C(=O)NC3CC(C)CCN(c4nnn[nH]4)C3)CC2)c(O)c1] |
| | | 1-(4-(difluoromethoxy)-2- | | | 1-(2-hydroxy-4-methylphenyl)-N-(5- |
| | | methoxyphenyl)-N-((3R,5S)-5- | | | methyl-1-(1H-tetrazol-5-yl)azepan-3- |
| | | methyl-1-(1H-tetrazol-5- | | | yl)cyclopropane-1-carboxamide |
| | | yl)piperidin-3-yl)cyclopropane-1- | | | |
| | | carboxamide | | | |
| 19B- OMe | 19 | 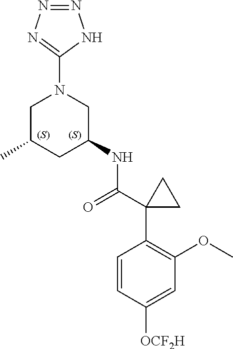 | 67A* | TA | 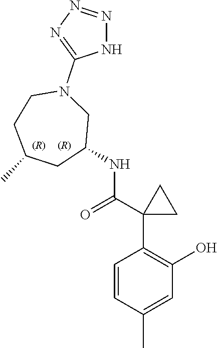 |
| | | 1-(4-(difluoromethoxy)-2- | | | 1-(2-hydroxy-4-methylphenyl)-N- |
| | | methoxyphenyl)-N-((3S,5S)-5- | | | ((3R,5R)-5-methyl-1-(1H-tetrazol-5- |
| | | methyl-1-(1H-tetrazol-5- | | | yl)azepan-3-yl)cyclopropane-1- |
| | | yl)piperidin-3-yl)cyclopropane-1- | | | carboxamide |
| | | carboxamide | | | |
| 19C- OMe | 19 | 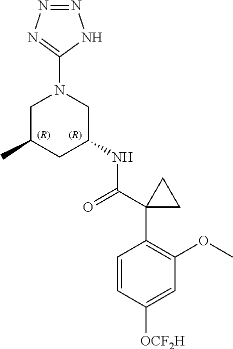 | 67B* | TA | 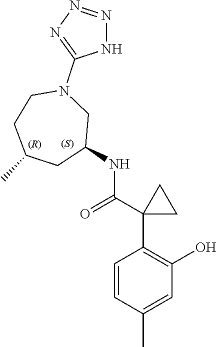 |
| | | 1-(4-(difluoromethoxy)-2- | | | 1-(2-hydroxy-4-methylphenyl)-N- |
| | | methoxyphenyl)-N-((3R,5R)-5- | | | ((3S,5R)-5-methyl-1-(1H-tetrazol-5- |
| | | methyl-1-(1H-tetrazol-5- | | | yl)azepan-3-yl)cyclopropane-1- |
| | | yl)piperidin-3-yl)cyclopropane-1- | | | carboxamide |
| | | carboxamide | | | |
| 19D- OMe | 19 | 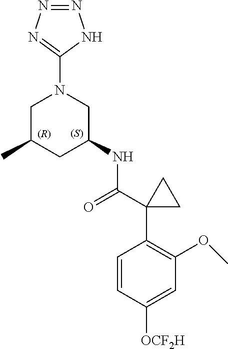 | 67C* | TA | 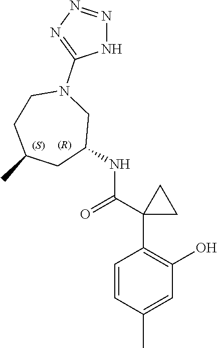 |
| | | 1-(4-(difluoromethoxy)-2- | | | 1-(2-hydroxy-4-methylphenyl)-N- |
| | | methoxyphenyl)-N-((3S,5R)-5- | | | ((3R,5S)-5-methyl-1-(1H-tetrazol-5- |
| | | methyl-1-(1H-tetrazol-5- | | | yl)azepan-3-yl)cyclopropane-1- |
| | | yl)piperidin-3-yl)cyclopropane-1- | | | carboxamide |
| | | carboxamide | | | |
| 20A | 20 | 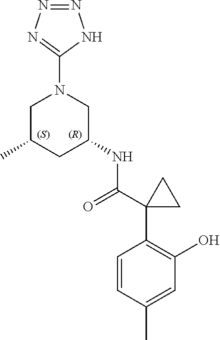 | 67D* | TA | 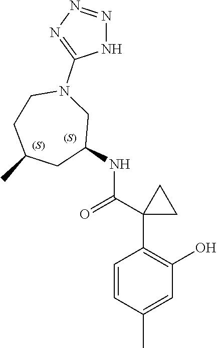 |
| | | 1-(2-hydroxy-4-methylphenyl)-N- | | | 1-(2-hydroxy-4-methylphenyl)-N- |
| | | ((3R,5S)-5-methyl-1-(1H-tetrazol- | | | ((3S,5S)-5-methyl-1-(1H-tetrazol-5- |
| | | 5-yl)piperidin-3-yl)cyclopropane- | | | yl)azepan-3-yl)cyclopropane-1- |
| | | 1-carboxamide | | | carboxamide |
| 20B | 20 | 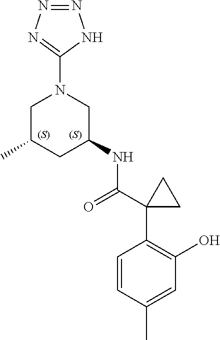 | Rac-68 | TA | 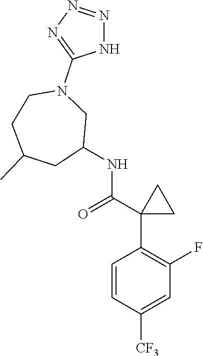 [SMILES: CC1CCN(c2nnn[nH]2)CC(NC(=O)C2(c3ccc(C(F)(F)F)cc3F)CC2)C1] |
| | | 1-(2-hydroxy-4-methylphenyl)-N- | | | 1-(2-fluoro-4-(trifluoromethyl)phenyl)- |
| | | ((3S,5S)-5-methyl-1-(1H-tetrazol- | | | N-(5-methyl-1-(1H-tetrazol-5-yl)azepan- |
| | | 5-yl)piperidin-3-yl)cyclopropane- | | | 3-yl)cyclopropane-1-carboxamide |
| | | 1-carboxamide | | | |
| 20C | 20 | 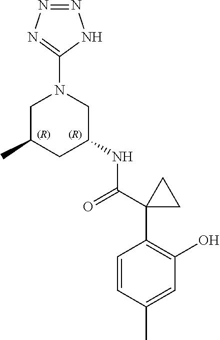 | 68A* | TA | 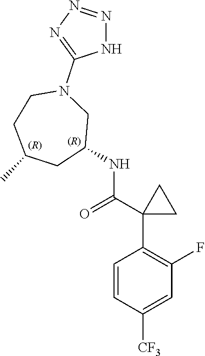 |
| | | 1-(2-hydroxy-4-methylphenyl)-N- | | | 1-(2-fluoro-4-(trifluoromethyl)phenyl)- |
| | | ((3R,5R)-5-methyl-1-(1H-tetrazol- | | | N-((3R,5R)-5-methyl-1-(1H-tetrazol-5- |
| | | 5-yl)piperidin-3-yl)cyclopropane- | | | yl)azepan-3-yl)cyclopropane-1- |
| | | 1-carboxamide | | | carboxamide |
| 20D | 20 | 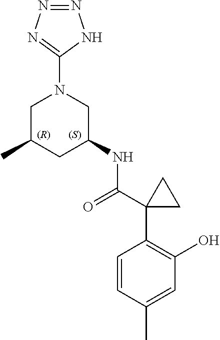 | 68B* | TA | 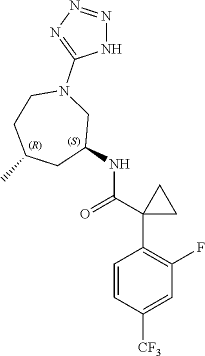 |
| | | 1-(2-hydroxy-4-methylphenyl)-N- | | | 1-(2-fluoro-4-(trifluoromethyl)phenyl)- |
| | | ((3S,5R)-5-methyl-1-(1H-tetrazol- | | | N-((3S,5R)-5-methyl-1-(1H-tetrazol-5- |
| | | 5-yl)piperidin-3-yl)cyclopropane- | | | yl)azepan-3-yl)cyclopropane-1- |
| | | 1-carboxamide | | | carboxamide |
| 20A- OMe | 20 | 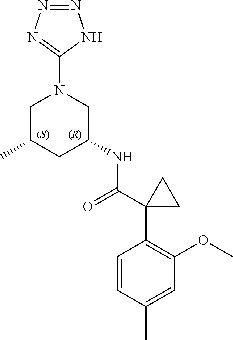 | 68C* | TA | 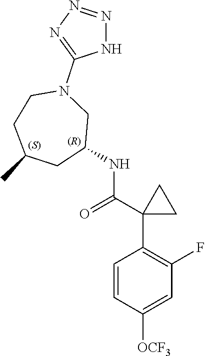 |
| | | 1-(2-methoxy-4-methylphenyl)-N- | | | 1-(2-fluoro-4-(trifluoromethyl)phenyl)- |
| | | ((3R,5S)-5-methyl-1-(1H-tetrazol- | | | N-((3R,5S)-5-methyl-1-(1H-tetrazol-5- |
| | | 5-yl)piperidin-3-yl)cyclopropane- | | | yl)azepan-3-yl)cyclopropane-1- |
| | | 1-carboxamide | | | carboxamide |
| 20B- OMe | 20 | 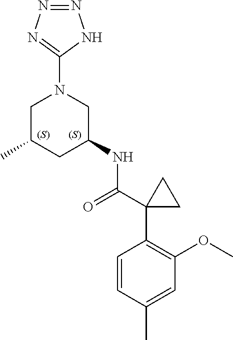 | 68D* | TA | 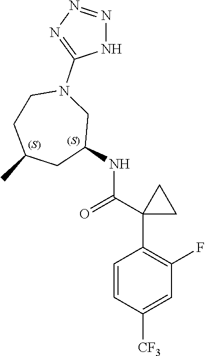 |
| | | 1-(2-methoxy-4-methylphenyl)-N- | | | 1-(2-fluoro-4-(trifluoromethyl)phenyl)- |
| | | ((3S,5S)-5-methyl-1-(1H-tetrazol- | | | N-((3S,5S)-5-methyl-1-(1H-tetrazol-5- |
| | | 5-yl)piperidin-3-yl)cyclopropane- | | | yl)azepan-3-yl)cyclopropane-1- |
| | | 1-carboxamide | | | carboxamide |
| 20C- OMe | 20 | 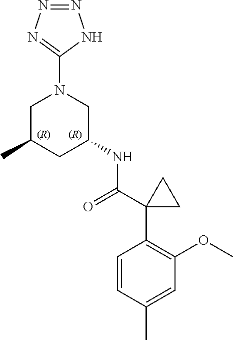 | Rac-69 | TA | 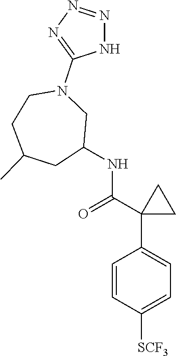 |
| | | 1-(2-methoxy-4-methylphenyl)-N- | | | N-(5-methyl-1-(1H-tetrazol-5-yl)azepan- |
| | | ((3R,5R)-5-methyl-1-(1H-tetrazol- | | | 3-yl)-1-(4- |
| | | 5-yl)piperidin-3-yl)cyclopropane- | | | ((trifluoromethyl)thio)phenyl) |
| | | 1-carboxamide | | | cyclopropane-1-carboxamide |
| 20D- OMe | 20 | 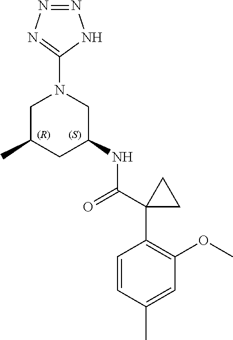 | 69A* | TA | 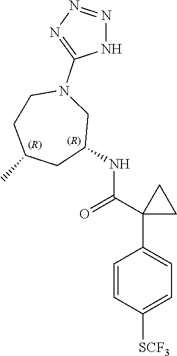 |
| | | 1-(2-methoxy-4-methylphenyl)-N- | | | N-((3R,5R)-5-methyl-1-(1H-tetrazol-5- |
| | | ((3S,5R)-5-methyl-1-(1H-tetrazol- | | | yl)azepan-3-yl)-1-(4- |
| | | 5-yl)piperidin-3-yl)cyclopropane- | | | ((trifluoromethyl)thio)phenyl) |
| | | 1-carboxamide | | | cyclopropane-1-carboxamide |
| 21A* | 21 | 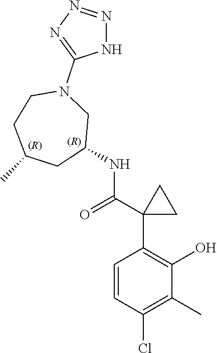 | 69B* | TA | 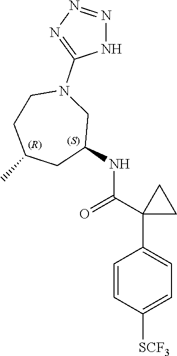 |
| | | 1-(4-chloro-2-hydroxy-3- | | | N-((3S,5R)-5-methyl-1-(1H-tetrazol-5- |
| | | methylphenyl)-N-((3R,5R)-5- | | | yl)azepan-3-yl)-1-(4- |
| | | methyl-1-(1H-tetrazol-5-yl)azepan- | | | ((trifluoromethyl)thio)phenyl) |
| | | 3-yl)cyclopropane-1-carboxamide | | | cyclopropane-1-carboxamide |
| 21B* | 21 | 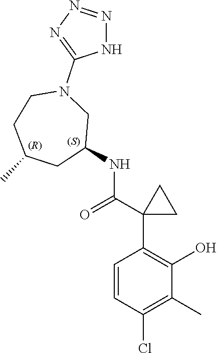 | 69C* | TA | 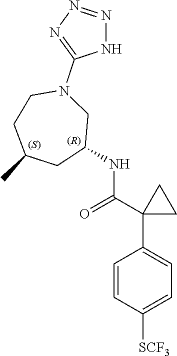 |
| | | 1-(4-chloro-2-hydroxy-3- | | | N-((3R,5S)-5-methyl-1-(1H-tetrazol-5- |
| | | methylphenyl)-N-((3S,5R)-5- | | | yl)azepan-3-yl)-1-(4- |
| | | methyl-1-(1H-tetrazol-5-yl)azepan- | | | ((trifluoromethyl)thio)phenyl) |
| | | 3-yl)cyclopropane-1-carboxamide | | | cyclopropane-1-carboxamide |
| 21C* | 21 | 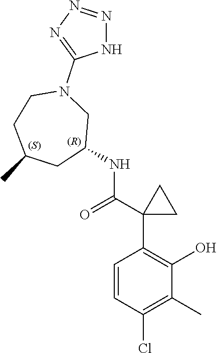 | 69D* | TA | 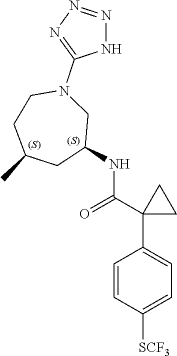 |
| | | 1-(4-chloro-2-hydroxy-3- | | | N-((3S,5S)-5-methyl-1-(1H-tetrazol-5- |
| | | methylphenyl)-N-((3R,5S)-5- | | | yl)azepan-3-yl)-1-(4- |
| | | methyl-1-(1H-tetrazol-5-yl)azepan- | | | ((trifluoromethyl)thio)phenyl) |
| | | 3-yl)cyclopropane-1-carboxamide | | | cyclopropane-1-carboxamide |
| 21D* | 21 | 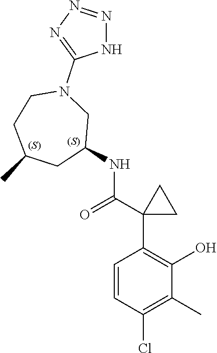 | Rac-70 | TA | 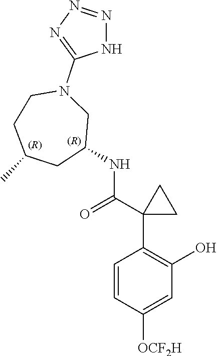 |
| | | 1-(4-chloro-2-hydroxy-3- | | | 1-(4-(difluoromethoxy)-2- |
| | | methylphenyl)-N-((3S,5S)-5- | | | hydroxyphenyl)-N-(5-methyl-1-(1H- |
| | | methyl-1-(1H-tetrazol-5-yl)azepan- | | | tetrazol-5-yl)azepan-3-yl)cyclopropane- |
| | | 3-yl)cyclopropane-1-carboxamide | | | 1-carboxamide |
| 21A- OMe* | 21 | 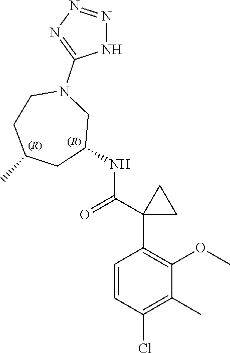 | 70A* | TA | 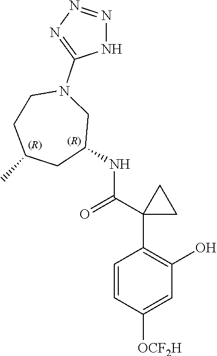 |
| | | 1-(4-chloro-2-methoxy-3- | | | 1-(4-(difluoromethoxy)-2- |
| | | methylphenyl)-N-((3R,5R)-5- | | | hydroxyphenyl)-N-((3R,5R)-5-methyl- |
| | | methyl-1-(1H-tetrazol-5-yl)azepan- | | | 1-(1H-tetrazol-5-yl)azepan-3- |
| | | 3-yl)cyclopropane-1-carboxamide | | | yl)cyclopropane-1-carboxamide |
| 21B- OMe* | 21 | 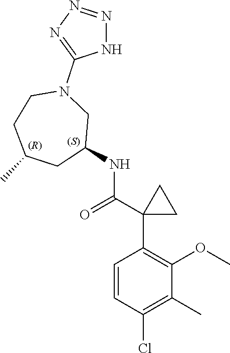 | 70B* | TA | 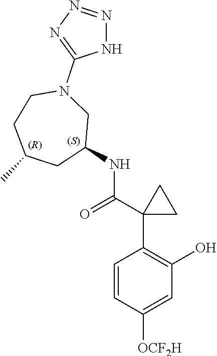 |
| | | 1-(4-chloro-2-methoxy-3- | | | 1-(4-(difluoromethoxy)-2- |
| | | methylphenyl)-N-((3S,5R)-5- | | | hydroxyphenyl)-N-((3S,5R)-5-methyl-1- |
| | | methyl-1-(1H-tetrazol-5-yl)azepan- | | | (1H-tetrazol-5-yl)azepan-3- |
| | | 3-yl)cyclopropane-1-carboxamide | | | yl)cyclopropane-1-carboxamide |
| 21C- OMe* | 21 | 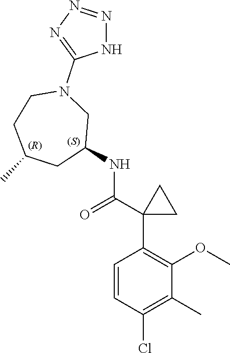 | 70C* | TA | 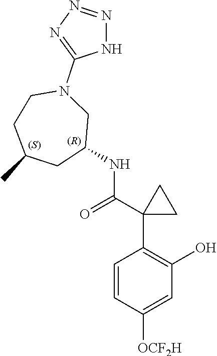 |
| | | 1-(4-chloro-2-methoxy-3- | | | 1-(4-(difluoromethoxy)-2- |
| | | methylphenyl)-N-((3R,5S)-5- | | | hydroxyphenyl)-N-((3R,5S)-5-methyl-1- |
| | | methyl-1-(1H-tetrazol-5-yl)azepan- | | | (1H-tetrazol-5-yl)azepan-3- |
| | | 3-yl)cyclopropane-1-carboxamide | | | yl)cyclopropane-1-carboxamide |
| 21D- OMe* | 21 | 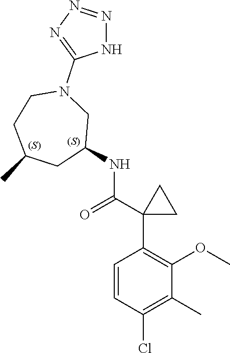 | 70D* | TA | 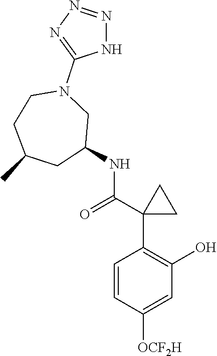 |
| | | 1-(4-chloro-2-methoxy-3- | | | 1-(4-(difluoromethoxy)-2- |
| | | methylphenyl)-N-((3S,5S)-5- | | | hydroxyphenyl)-N-((3S,5S)-5-methyl-1- |
| | | methyl-1-(1H-tetrazol-5-yl)azepan- | | | (1H-tetrazol-5-yl)azepan-3- |
| | | 3-yl)cyclopropane-1-carboxamide | | | yl)cyclopropane-1-carboxamide |
| 22A* | 22 | 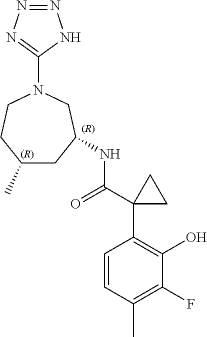 | Rac-71 | TA | 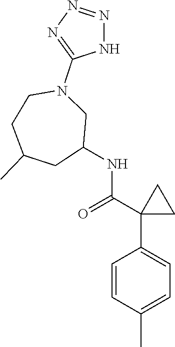 [SMILES: Cc1ccc(C2(C(=O)NC3CC(C)CCN(c4nnn[nH]4)C3)CC2)cc1] |
| | | 1-(3-fluoro-2-hydroxy-4- | | | N-(5-methyl-1-(1H-tetrazol-5-yl)azepan- |
| | | methylphenyl)-N-((3R,5R)-5- | | | 3-yl)-1-(p-tolyl)cyclopropane-1- |
| | | methyl-1-(1H-tetrazol-5-yl)azepan- | | | carboxamide |
| | | 3-yl)cyclopropane-1-carboxamide | | | |
| 22B* | 22 | 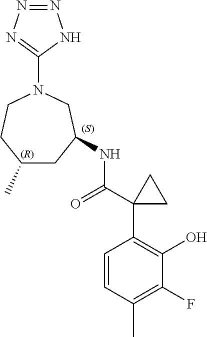 | 71A* | TA | 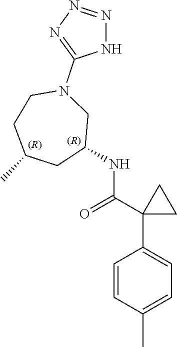 |
| | | 1-(3-fluoro-2-hydroxy-4- | | | N-((3R,5R)-5-methyl-1-(1H-tetrazol-5- |
| | | methylphenyl)-N-((3S,5R)-5- | | | yl)azepan-3-yl)-1-(p-tolyl)cyclopropane- |
| | | methyl-1-(1H-tetrazol-5-yl)azepan- | | | 1-carboxamide |
| | | 3-yl)cyclopropane-1-carboxamide | | | |
| 22C* | 22 | 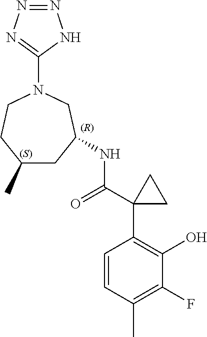 | 71B* | TA | 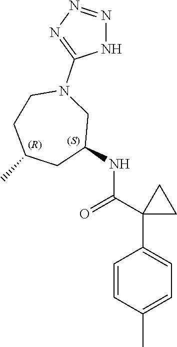 |
| | | 1-(3-fluoro-2-hydroxy-4- | | | N-((3S,5R)-5-methyl-1-(1H-tetrazol-5- |
| | | methylphenyl)-N-((3R,5S)-5- | | | yl)azepan-3-yl)-1-(p-tolyl)cyclopropane- |
| | | methyl-1-(1H-tetrazol-5-yl)azepan- | | | 1-carboxamide |
| | | 3-yl)cyclopropane-1-carboxamide | | | |
| 22D* | 22 | 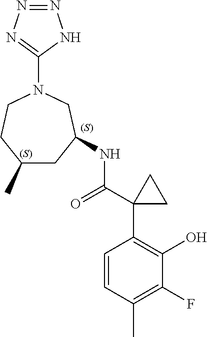 | 71C* | TA | 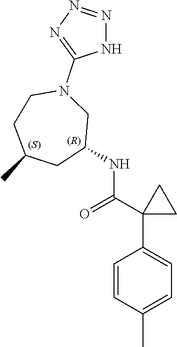 |
| | | 1-(3-fluoro-2-hydroxy-4- | | | N-((3R,5S)-5-methyl-1-(1H-tetrazol-5- |
| | | methylphenyl)-N-((3S,5S)-5- | | | yl)azepan-3-yl)-1-(p-tolyl)cyclopropane- |
| | | methyl-1-(1H-tetrazol-5-yl)azepan- | | | 1-carboxamide |
| | | 3-yl)cyclopropane-1-carboxamide | | | |
| 22A- OMe* | 22 | 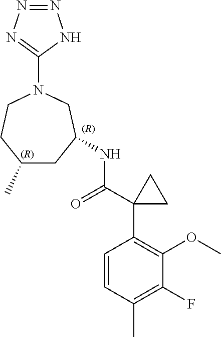 | 71D* | TA | 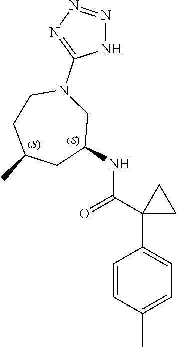 |
| | | 1-(3-fluoro-2-methoxy-4- | | | N-((3S,5S)-5-methyl-1-(1H-tetrazol-5- |
| | | methylphenyl)-N-((3R,5R)-5- | | | yl)azepan-3-yl)-1-(p-tolyl)cyclopropane- |
| | | methyl-1-(1H-tetrazol-5-yl)azepan- | | | 1-carboxamide |
| | | 3-yl)cyclopropane-1-carboxamide | | | |
| 22B- OMe* | 22 | 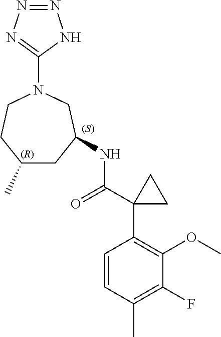 | Rac-72 | TA | 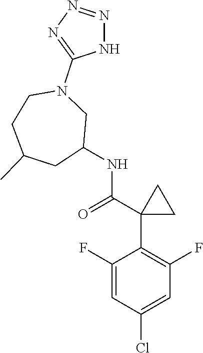 [SMILES: CC1CCN(c2nnn[nH]2)CC(NC(=O)C2(c3c(F)cc(Cl)cc3F)CC2)C1] |
| | | 1-(3-fluoro-2-methoxy-4- | | | 1-(4-chloro-2,6-difluorophenyl)-N-(5- |
| | | methylphenyl)-N-((3S,5R)-5- | | | methyl-1-(1H-tetrazol-5-yl)azepan-3- |
| | | methyl-1-(1H-tetrazol-5-yl)azepan- | | | yl)cyclopropane-1-carboxamide |
| | | 3-yl)cyclopropane-1-carboxamide | | | |
| 22C- OMe* | 22 | 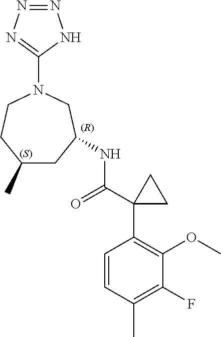 | 72A* | TA | 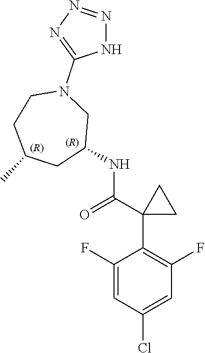 |
| | | 1-(3-fluoro-2-methoxy-4- | | | 1-(4-chloro-2,6-difluorophenyl)-N- |
| | | methylphenyl)-N-((3R,5S)-5- | | | ((3R,5R)-5-methyl-1-(1H-tetrazol-5- |
| | | methyl-1-(1H-tetrazol-5-yl)azepan- | | | yl)azepan-3-yl)cyclopropane-1- |
| | | 3-yl)cyclopropane-1-carboxamide | | | carboxamide |
| 22D- OMe* | 22 | 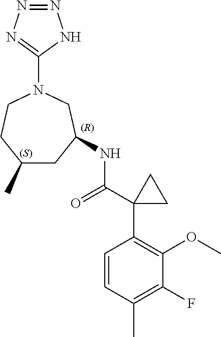 | 72B* | TA | 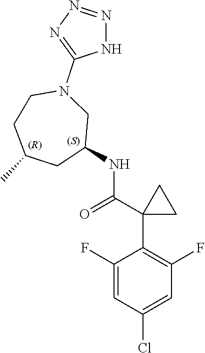 |
| | | 1-(3-fluoro-2-methoxy-4- | | | 1-(4-chloro-2,6-difluorophenyl)-N- |
| | | methylphenyl)-N-((3S,5S)-5- | | | ((3S,5R)-5-methyl-1-(1H-tetrazol-5- |
| | | methyl-1-(1H-tetrazol-5-yl)azepan- | | | yl)azepan-3-yl)cyclopropane-1- |
| | | 3-yl)cyclopropane-1-carboxamide | | | carboxamide |
| 23A | 23 | 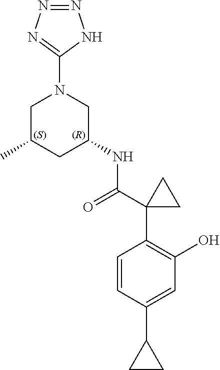 | 72C* | TA | 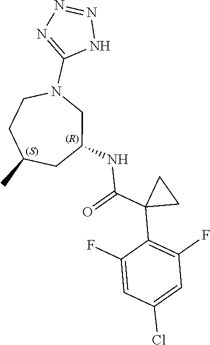 |
| | | 1-(4-cyclopropyl-2- | | | 1-(4-chloro-2,6-difluorophenyl)-N- |
| | | hydroxyphenyl)-N-((3R,5S)-5- | | | ((3R,5S)-5-methyl-1-(1H-tetrazol-5- |
| | | methyl-1-(1H-tetrazol-5- | | | yl)azepan-3-yl)cyclopropane-1- |
| | | yl)piperidin-3-yl)cyclopropane-1- | | | carboxamide |
| | | carboxamide | | | |
| 23B | 23 | 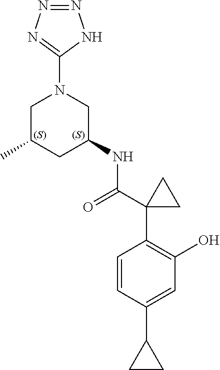 | 72D* | TA | 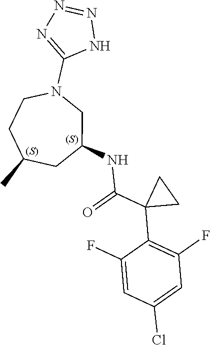 |
| | | 1-(4-cyclopropyl-2- | | | 1-(4-chloro-2,6-difluorophenyl)-N- |
| | | hydroxyphenyl)-N-((3S,5S)-5- | | | ((3S,5S)-5-methyl-1-(1H-tetrazol-5- |
| | | methyl-1-(1H-tetrazol-5- | | | yl)azepan-3-yl)cyclopropane-1- |
| | | yl)piperidin-3-yl)cyclopropane-1- | | | carboxamide |
| | | carboxamide | | | |
| 23C | 23 | 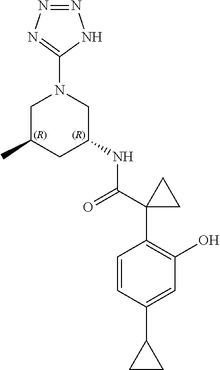 | Rac-73 | TA | 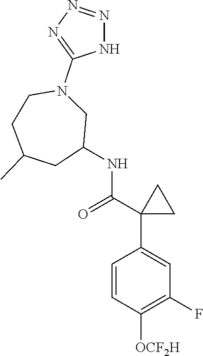 |
| | | 1-(4-cyclopropyl-2- | | | 1-(4-(difluoromethoxy)-3-fluorophenyl)- |
| | | hydroxyphenyl)-N-((3R,5R)-5- | | | N-(5-methyl-1-(1H-tetrazol-5-yl)azepan- |
| | | methyl-1-(1H-tetrazol-5- | | | 3-yl)cyclopropane-1-carboxamide |
| | | yl)piperidin-3-yl)cyclopropane-1- | | | |
| | | carboxamide | | | |
| 23D | 23 | 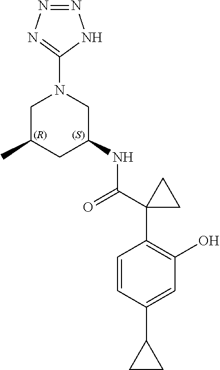 | 73A* | TA | 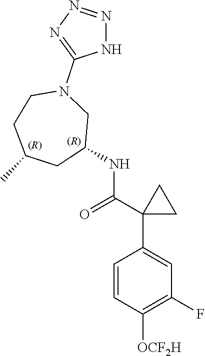 |
| | | 1-(4-cyclopropyl-2- | | | 1-(4-(difluoromethoxy)-3-fluorophenyl)- |
| | | hydroxyphenyl)-N-((3S,5R)-5- | | | N-((3R,5R)-5-methyl-1-(1H-tetrazol-5- |
| | | methyl-1-(1H-tetrazol-5- | | | yl)azepan-3-yl)cyclopropane-1- |
| | | yl)piperidin-3-yl)cyclopropane-1- | | | carboxamide |
| | | carboxamide | | | |
| 23A- OMe | 23 | 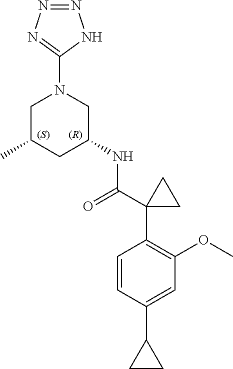 | 73B* | TA | 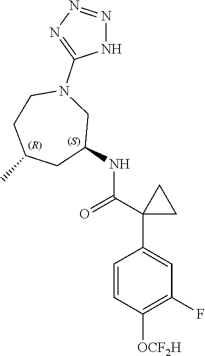 |
| | | 1-(4-cyclopropyl-2- | | | 1-(4-(difluoromethoxy)-3-fluorophenyl)- |
| | | methoxyphenyl)-N-((3R,5S)-5- | | | N-((3S,5R)-5-methyl-1-(1H-tetrazol-5- |
| | | methyl-1-(1H-tetrazol-5- | | | yl)azepan-3-yl)cyclopropane-1- |
| | | yl)piperidin-3-yl)cyclopropane-1- | | | carboxamide |
| | | carboxamide | | | |
| 23B- OMe | 23 | 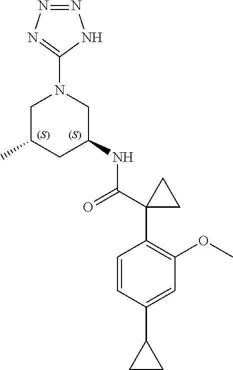 | 73C* | TA | 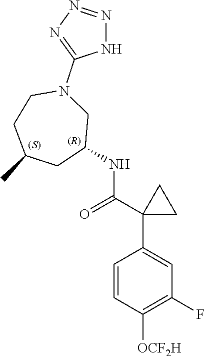 |
| | | 1-(4-cyclopropyl-2- | | | 1-(4-(difluoromethoxy)-3-fluorophenyl)- |
| | | methoxyphenyl)-N-((3S,5S)-5- | | | N-((3R,5S)-5-methyl-1-(1H-tetrazol-5- |
| | | methyl-1-(1H-tetrazol-5- | | | yl)azepan-3-yl)cyclopropane-1- |
| | | yl)piperidin-3-yl)cyclopropane-1- | | | carboxamide |
| | | carboxamide | | | |
| 23C- OMe | 23 | 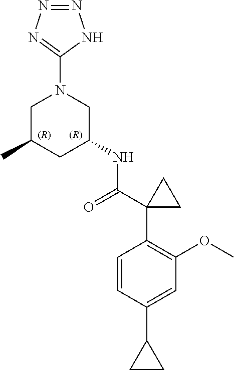 | 73D* | TA | 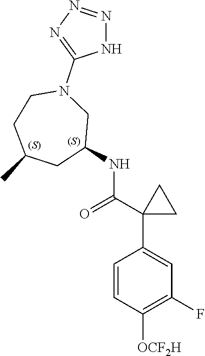 |
| | | 1-(4-cyclopropyl-2- | | | 1-(4-(difluoromethoxy)-3-fluorophenyl)- |
| | | methoxyphenyl)-N-((3R,5R)-5- | | | N-((3S,5S)-5-methyl-1-(1H-tetrazol-5- |
| | | methyl-1-(1H-tetrazol-5- | | | yl)azepan-3-yl)cyclopropane-1- |
| | | yl)piperidin-3-yl)cyclopropane-1- | | | carboxamide |
| | | carboxamide | | | |
| 23D- OMe | 23 | 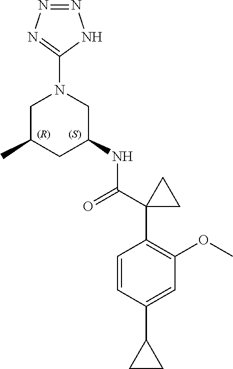 | Rac-74 | TA | 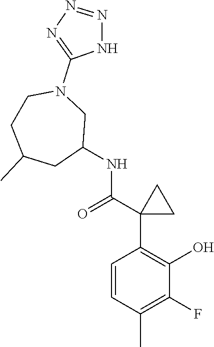 [SMILES: Cc1ccc(C2(C(=O)NC3CC(C)CCN(c4nnn[nH]4)C3)CC2)c(O)c1F] |
| | | 1-(4-cyclopropyl-2- | | | 1-(3-fluoro-2-hydroxy-4-methylphenyl)- |
| | | methoxyphenyl)-N-((3S,5R)-5- | | | N-(5-methyl-1-(1H-tetrazol-5-yl)azepan- |
| | | methyl-1-(1H-tetrazol-5- | | | 3-yl)cyclopropane-1-carboxamide |
| | | yl)piperidin-3-yl)cyclopropane-1- | | | |
| | | carboxamide | | | |
| 24A | 24 | 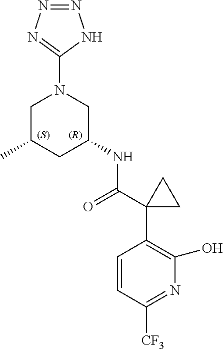 | 74A* | TA | 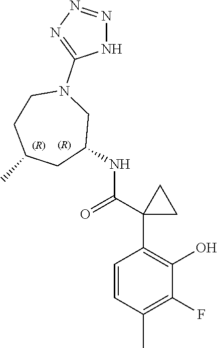 |
| | | 1-(2-hydroxy-6- | | | 1-(3-fluoro-2-hydroxy-4-methylphenyl)- |
| | | (trifluoromethyl)pyridin-3-yl)-N- | | | N-((3R,5R)-5-methyl-1-(1H-tetrazol-5- |
| | | ((3R,5S)-5-methyl-1-(1H-tetrazol- | | | yl)azepan-3-yl)cyclopropane-1- |
| | | 5-yl)piperidin-3-yl)cyclopropane- | | | carboxamide |
| | | 1-carboxamide | | | |
| 24B | 24 | 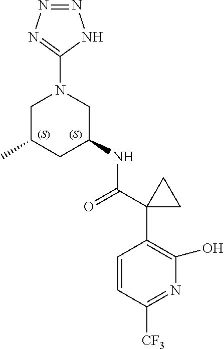 | 74B* | TA | 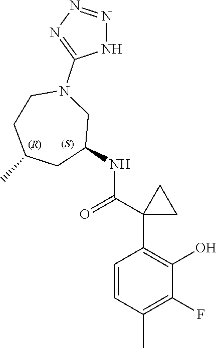 |
| | | 1-(2-hydroxy-6- | | | 1-(3-fluoro-2-hydroxy-4-methylphenyl)- |
| | | (trifluoromethyl)pyridin-3-yl)-N- | | | N-((3S,5R)-5-methyl-1-(1H-tetrazol-5- |
| | | ((3S,5S)-5-methyl-1-(1H-tetrazol- | | | yl)azepan-3-yl)cyclopropane-1- |
| | | 5-yl)piperidin-3-yl)cyclopropane- | | | carboxamide |
| | | 1-carboxamide | | | 1111. |
| 24C | 24 | 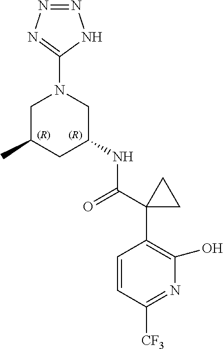 | 74C* | TA | 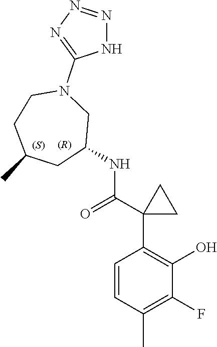 |
| | | 1-(2-hydroxy-6- | | | 1-(3-fluoro-2-hydroxy-4-methylphenyl)- |
| | | (trifluoromethyl)pyridin-3-yl)-N- | | | N-((3R,5S)-5-methyl-1-(1H-tetrazol-5- |
| | | ((3R,5R)-5-methyl-1-(1H-tetrazol- | | | yl)azepan-3-yl)cyclopropane-1- |
| | | 5-yl)piperidin-3-yl)cyclopropane- | | | carboxamide |
| | | 1-carboxamide | | | |
| 24D | 24 | 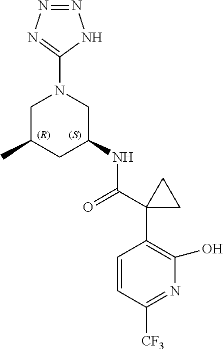 | 74D* | TA | 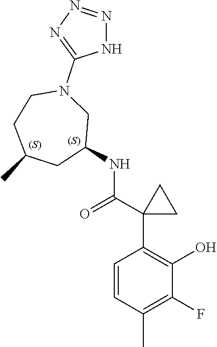 |
| | | 1-(2-hydroxy-6- | | | 1-(3-fluoro-2-hydroxy-4-methylphenyl)- |
| | | (trifluoromethyl)pyridin-3-yl)-N- | | | N-((3S,5S)-5-methyl-1-(1H-tetrazol-5- |
| | | ((3S,5R)-5-methyl-1-(1H-tetrazol- | | | yl)azepan-3-yl)cyclopropane-1- |
| | | 5-yl)piperidin-3-yl)cyclopropane- | | | carboxamide |
| | | 1-carboxamide | | | |
| 24A- OMe | 24 | 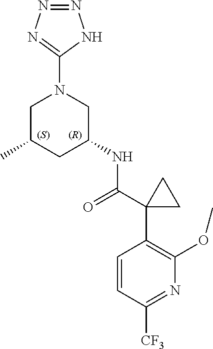 | 75A* | TA | 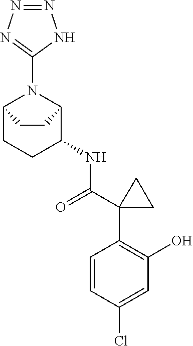 [SMILES: O=C(N[C@@H]1CC[C@@H]2CC[C@H]1N2c1nnn[nH]1)C1(c2ccc(Cl)cc2O)CC1] |
| | | 1-(2-methoxy-6- | | | N-((1R,2R,5R)-8-(1H-tetrazol-5-yl)-8- |
| | | (trifluoromethyl)pyridin-3-yl)-N- | | | azabicyclo[3.2.1]octan-2-yl)-1-(4- |
| | | ((3R,5S)-5-methyl-1-(1H-tetrazol- | | | chloro-2-hydroxyphenyl)cyclopropane- |
| | | 5-yl)piperidin-3-yl)cyclopropane- | | | 1-carboxamide |
| | | 1-carboxamide | | | |
| 24B- OMe | 24 | 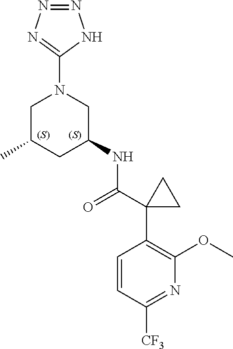 | 75B* | TA | 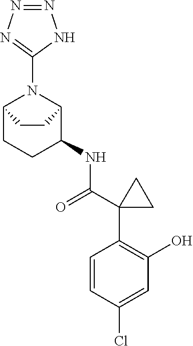 [SMILES: O=C(N[C@H]1CC[C@@H]2CC[C@H]1N2c1nnn[nH]1)C1(c2ccc(Cl)cc2O)CC1] |
| | | 1-(2-methoxy-6- | | | N-((1R,2S,5R)-8-(1H-tetrazol-5-yl)-8- |
| | | (trifluoromethyl)pyridin-3-yl)-N- | | | azabicyclo[3.2.1]octan-2-yl)-1-(4- |
| | | ((3S,5S)-5-methyl-1-(1H-tetrazol- | | | chloro-2-hydroxyphenyl)cyclopropane- |
| | | 5-yl)piperidin-3-yl)cyclopropane- | | | 1-carboxamide |
| | | 1-carboxamide | | | |
| 24C- OMe | 24 | 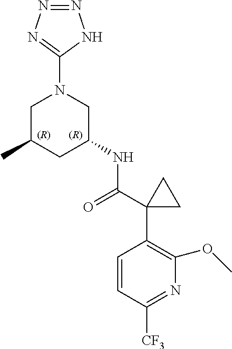 | 75C* | TA | 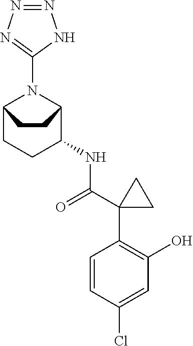 |
| | | 1-(2-methoxy-6- | | | N-((1S,2R,5S)-8-(1H-tetrazol-5-yl)-8- |
| | | (trifluoromethyl)pyridin-3-yl)-N- | | | azabicyclo[3.2.1]octan-2-yl)-1-(4- |
| | | ((3R,5R)-5-methyl-1-(1H-tetrazol- | | | chloro-2-hydroxyphenyl)cyclopropane- |
| | | 5-yl)piperidin-3-yl)cyclopropane- | | | 1-carboxamide |
| | | 1-carboxamide | | | |
| 24D- OMe | 24 | 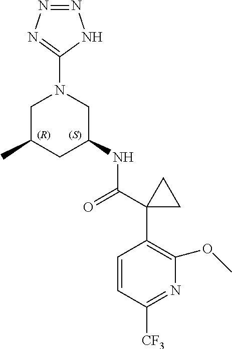 | 75D* | TA | 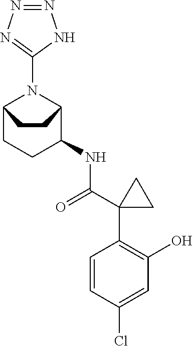 |
| | | 1-(2-methoxy-6- | | | N-((1S,2S,5S)-8-(1H-tetrazol-5-yl)-8- |
| | | (trifluoromethyl)pyridin-3-yl)-N- | | | azabicyclo[3.2.1]octan-2-yl)-1-(4- |
| | | ((3S,5R)-5-methyl-1-(1H-tetrazol- | | | chloro-2-hydroxyphenyl)cyclopropane- |
| | | 5-yl)piperidin-3-yl)cyclopropane- | | | 1-carboxamide |
| | | 1-carboxamide | | | |
| 25A | 25 | 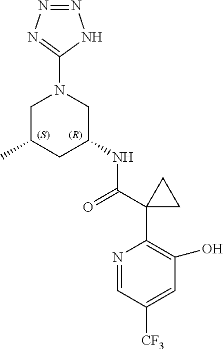 | 76A* | TA | 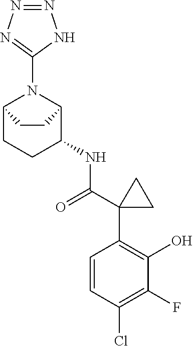 [SMILES: O=C(N[C@@H]1CC[C@@H]2CC[C@H]1N2c1nnn[nH]1)C1(c2ccc(Cl)c(F)c2O)CC1] |
| | | 1-(3-hydroxy-5- | | | N-((1R,2R,5R)-8-(1H-tetrazol-5-yl)-8- |
| | | (trifluoromethyl)pyridin-2-yl)-N- | | | azabicyclo[3.2.1]octan-2-yl)-1-(4- |
| | | ((3R,5S)-5-methyl-1-(1H-tetrazol- | | | chloro-3-fluoro-2- |
| | | 5-yl)piperidin-3-yl)cyclopropane- | | | hydroxyphenyl)cyclopropane-1- |
| | | 1-carboxamide | | | carboxamide |
| 25B | 25 | 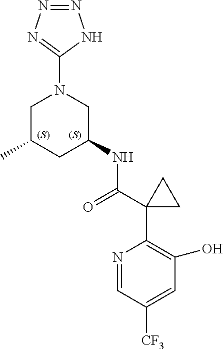 | 76B* | TA | 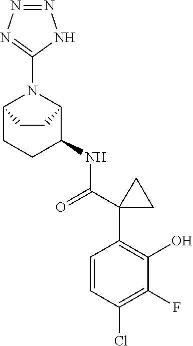 [SMILES: O=C(N[C@H]1CC[C@@H]2CC[C@H]1N2c1nnn[nH]1)C1(c2ccc(Cl)c(F)c2O)CC1] |
| | | 1-(3-hydroxy-5- | | | N-((1R,2S,5R)-8-(1H-tetrazol-5-yl)-8- |
| | | (trifluoromethyl)pyridin-2-yl)-N- | | | azabicyclo[3.2.1]octan-2-yl)-1-(4- |
| | | ((3S,5S)-5-methyl-1-(1H-tetrazol- | | | chloro-3-fluoro-2- |
| | | 5-yl)piperidin-3-yl)cyclopropane- | | | hydroxyphenyl)cyclopropane-1- |
| | | 1-carboxamide | | | carboxamide |
| 25C | 25 | 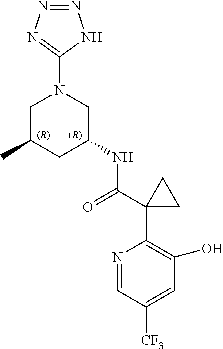 | 76C* | TA | 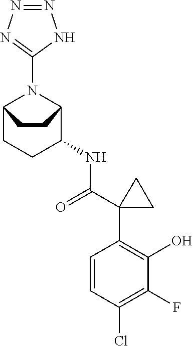 |
| | | 1-(3-hydroxy-5- | | | N-((1S,2R,5S)-8-(1H-tetrazol-5-yl)-8- |
| | | (trifluoromethyl)pyridin-2-yl)-N- | | | azabicyclo[3.2.1]octan-2-yl)-1-(4- |
| | | ((3R,5R)-5-methyl-1-(1H-tetrazol- | | | chloro-3-fluoro-2- |
| | | 5-yl)piperidin-3-yl)cyclopropane- | | | hydroxyphenyl)cyclopropane-1- |
| | | 1-carboxamide | | | carboxamide |
| 25D | 25 | 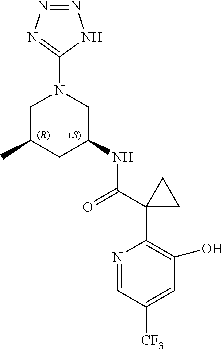 | 76D* | TA | 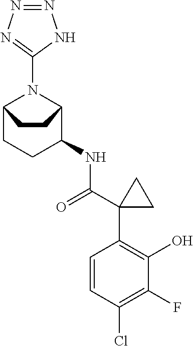 |
| | | 1-(3-hydroxy-5- | | | N-((1S,2S,5S)-8-(1H-tetrazol-5-yl)-8- |
| | | (trifluoromethyl)pyridin-2-yl)-N- | | | azabicyclo[3.2.1]octan-2-yl)-1-(4- |
| | | ((3S,5R)-5-methyl-1-(1H-tetrazol- | | | chloro-3-fluoro-2- |
| | | 5-yl)piperidin-3-yl)cyclopropane- | | | hydroxyphenyl)cyclopropane-1- |
| | | 1-carboxamide | | | carboxamide |
| 25A- OMe | 25 | 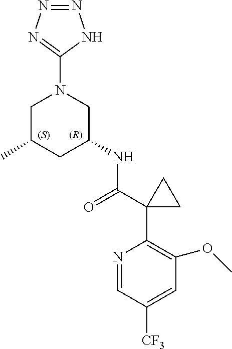 | 77A* | TA | 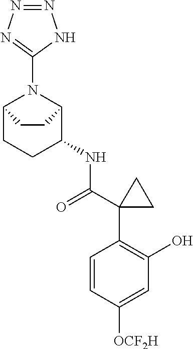 |
| | | 1-(3-methoxy-5- | | | N-((1R,2R,5R)-8-(1H-tetrazol-5-yl)-8- |
| | | (trifluoromethyl)pyridin-2-yl)-N- | | | azabicyclo[3.2.1]octan-2-yl)-1-(4- |
| | | ((3R,5S)-5-methyl-1-(1H-tetrazol- | | | (difluoromethoxy)-2- |
| | | 5-yl)piperidin-3-yl)cyclopropane- | | | hydroxyphenyl)cyclopropane-1- |
| | | 1-carboxamide | | | carboxamide |
| 25B- OMe | 25 | 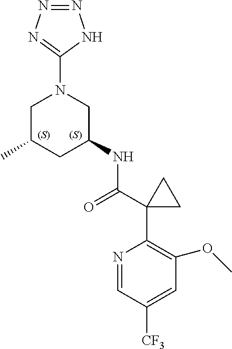 | 77B* | TA | 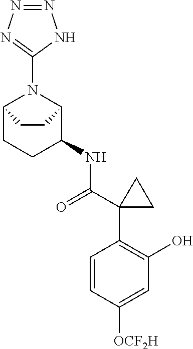 |
| | | 1-(3-methoxy-5- | | | N-((1R,2S,5R)-8-(1H-tetrazol-5-yl)-8- |
| | | (trifluoromethyl)pyridin-2-yl)-N- | | | azabicyclo[3.2.1]octan-2-yl)-1-(4- |
| | | ((3S,5S)-5-methyl-1-(1H-tetrazol- | | | (difluoromethoxy)-2- |
| | | 5-yl)piperidin-3-yl)cyclopropane- | | | hydroxyphenyl)cyclopropane-1- |
| | | 1-carboxamide | | | carboxamide |
| 25C- OMe | 25 | 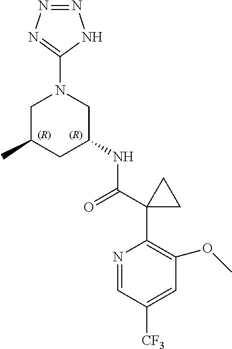 | 77C* | TA | 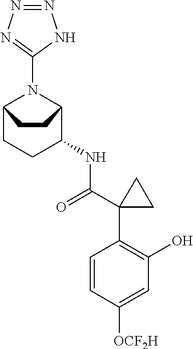 |
| | | 1-(3-methoxy-5- | | | N-((1S,2R,5S)-8-(1H-tetrazol-5-yl)-8- |
| | | (trifluoromethyl)pyridin-2-yl)-N- | | | azabicyclo[3.2.1]octan-2-yl)-1-(4- |
| | | ((3R,5R)-5-methyl-1-(1H-tetrazol- | | | (difluoromethoxy)-2- |
| | | 5-yl)piperidin-3-yl)cyclopropane- | | | hydroxyphenyl)cyclopropane-1- |
| | | 1-carboxamide | | | carboxamide |
| 25D- OMe | 25 | 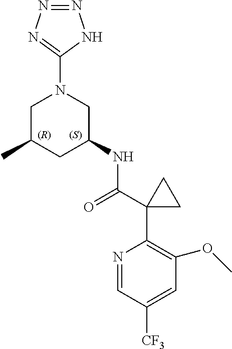 | 77D* | TA | 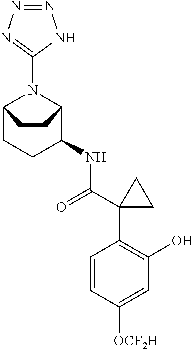 |
| | | 1-(3-methoxy-5- | | | N-((1S,2S,5S)-8-(1H-tetrazol-5-yl)-8- |
| | | (trifluoromethyl)pyridin-2-yl)-N- | | | azabicyclo[3.2.1]octan-2-yl)-1-(4- |
| | | ((3S,5R)-5-methyl-1-(1H-tetrazol- | | | (difluoromethoxy)-2- |
| | | 5-yl)piperidin-3-yl)cyclopropane- | | | hydroxyphenyl)cyclopropane-1- |
| | | 1-carboxamide | | | carboxamide |
| 26A | 26 | 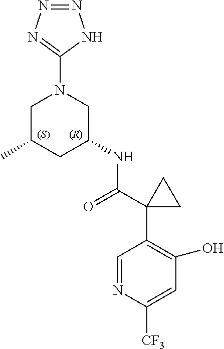 | 78A* | TA | 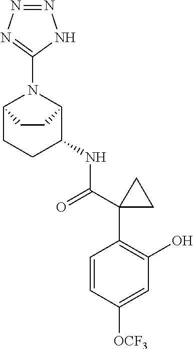 [SMILES: O=C(N[C@@H]1CC[C@@H]2CC[C@H]1N2c1nnn[nH]1)C1(c2ccc(OC(F)(F)F)cc2O)CC1] |
| | | 1-(4-hydroxy-6- | | | N-((1R,2R,5R)-8-(1H-tetrazol-5-yl)-8- |
| | | (trifluoromethyl)pyridin-3-yl)-N- | | | azabicyclo[3.2.1]octan-2-yl)-1-(2- |
| | | ((3R,5S)-5-methyl-1-(1H-tetrazol- | | | hydroxy-4- |
| | | 5-yl)piperidin-3-yl)cyclopropane- | | | (trifluoromethoxy)phenyl)cyclopropane- |
| | | 1-carboxamide | | | 1-carboxamide |
| 26B | 26 | 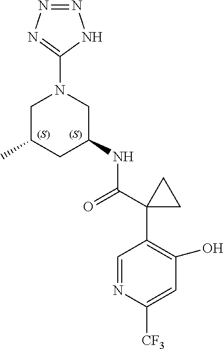 | 78B* | TA | 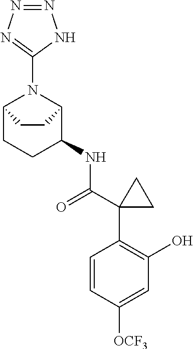 [SMILES: O=C(N[C@H]1CC[C@@H]2CC[C@H]1N2c1nnn[nH]1)C1(c2ccc(OC(F)(F)F)cc2O)CC1] |
| | | 1-(4-hydroxy-6- | | | N-((1R,2S,5R)-8-(1H-tetrazol-5-yl)-8- |
| | | (trifluoromethyl)pyridin-3-yl)-N- | | | azabicyclo[3.2.1]octan-2-yl)-1-(2- |
| | | ((3S,5S)-5-methyl-1-(1H-tetrazol- | | | hydroxy-4- |
| | | 5-yl)piperidin-3-yl)cyclopropane- | | | (trifluoromethoxy)phenyl)cyclopropane- |
| | | 1-carboxamide | | | 1-carboxamide |
| 26C | 26 | 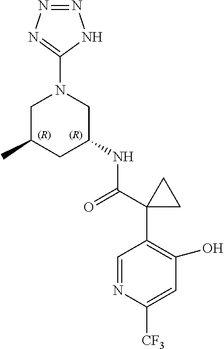 | 78C* | TA | 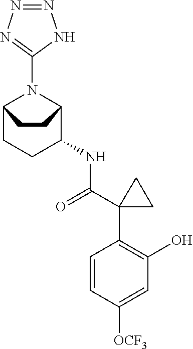 |
| | | 1-(4-hydroxy-6- | | | N-((1S,2R,5S)-8-(1H-tetrazol-5-yl)-8- |
| | | (trifluoromethyl)pyridin-3-yl)-N- | | | azabicyclo[3.2.1]octan-2-yl)-1-(2- |
| | | ((3R,5R)-5-methyl-1-(1H-tetrazol- | | | hydroxy-4- |
| | | 5-yl)piperidin-3-yl)cyclopropane- | | | (trifluoromethoxy)phenyl)cyclopropane- |
| | | 1-carboxamide | | | 1-carboxamide |
| 26D | 26 | 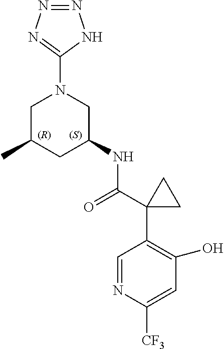 | 78D* | TA | 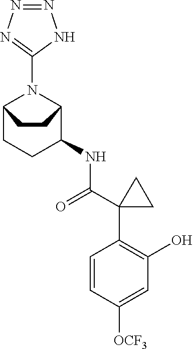 |
| | | 1-(4-hydroxy-6- | | | N-((1S,2S,5S)-8-(1H-tetrazol-5-yl)-8- |
| | | (trifluoromethyl)pyridin-3-yl)-N- | | | azabicyclo[3.2.1]octan-2-yl)-1-(2- |
| | | ((3S,5R)-5-methyl-1-(1H-tetrazol- | | | hydroxy-4- |
| | | 5-yl)piperidin-3-yl)cyclopropane- | | | (trifluoromethoxy)phenyl)cyclopropane- |
| | | 1-carboxamide | | | 1-carboxamide |
| 26A- OMe | 26 | 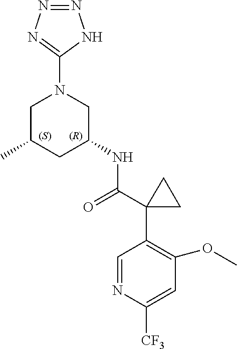 | Rac-79 | TA | 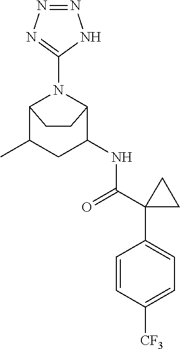 [SMILES: CC1CC(NC(=O)C2(c3ccc(C(F)(F)F)cc3)CC2)C2CCC1N2c1nnn[nH]1] |
| | | 1-(4-methoxy-6- | | | N-(4-methyl-8-(1H-tetrazol-5-yl)-8- |
| | | (trifluoromethyl)pyridin-3-yl)-N- | | | azabicyclo[3.2.1]octan-2-yl)-1-(4- |
| | | ((3R,5S)-5-methyl-1-(1H-tetrazol- | | | (trifluoromethyl)phenyl)cyclopropane-1- |
| | | 5-yl)piperidin-3-yl)cyclopropane- | | | carboxamide |
| | | 1-carboxamide | | | |
| 26B- OMe | 26 | 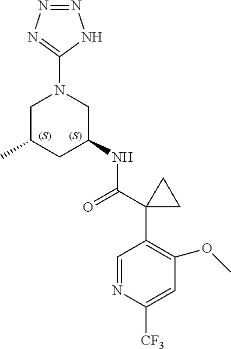 | 79′* | TA | 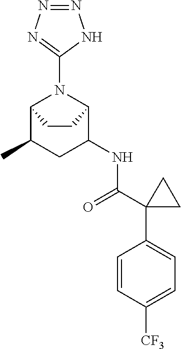 [SMILES: C[C@@H]1CC(NC(=O)C2(c3ccc(C(F)(F)F)cc3)CC2)[C@H]2CC[C@@H]1N2c1nnn[nH]1] |
| | | 1-(4-methoxy-6- | | | N-((1R,2R,5S)-4-methyl-8-(1H-tetrazol- |
| | | (trifluoromethyl)pyridin-3-yl)-N- | | | 5-yl)-8-azabicyclo[3.2.1]octan-2-yl)-1- |
| | | ((3S,5S)-5-methyl-1-(1H-tetrazol- | | | (4-(trifluoromethyl) |
| | | 5-yl)piperidin-3-yl)cyclopropane- | | | phenyl)cyclopropane-1- |
| | | 1-carboxamide | | | carboxamide |
| 26C- OMe | 26 | 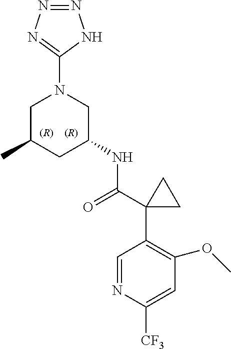 | 79″* | TA | 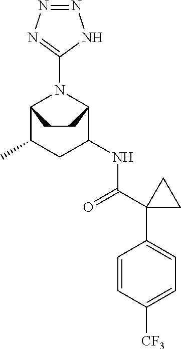 [SMILES: C[C@H]1CC(NC(=O)C2(c3ccc(C(F)(F)F)cc3)CC2)[C@@H]2CC[C@H]1N2c1nnn[nH]1] |
| | | 1-(4-methoxy-6- | | | N-((1S,2S,5R)-4-methyl-8-(1H-tetrazol- |
| | | (trifluoromethyl)pyridin-3-yl)-N- | | | 5-yl)-8-azabicyclo[3.2.1]octan-2-yl)-1- |
| | | ((3R,5R)-5-methyl-1-(1H-tetrazol- | | | (4-(trifluoromethyl) |
| | | 5-yl)piperidin-3-yl)cyclopropane- | | | phenyl)cyclopropane-1- |
| | | 1-carboxamide | | | carboxamide |
| 26D- OMe | 26 | 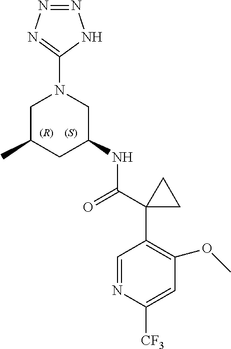 | 79A* | TA | 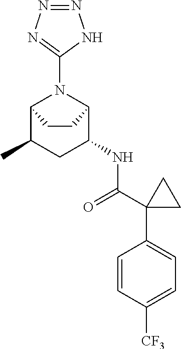 [SMILES: C[C@@H]1C[C@@H](NC(=O)C2(c3ccc(C(F)(F)F)cc3)CC2)[C@H]2CC[C@@H]1N2c1nnn[nH]1] |
| | | 1-(4-methoxy-6- | | | N-((1R,2R,4R,5S)-4-methyl-8-(1H- |
| | | (trifluoromethyl)pyridin-3-yl)-N- | | | tetrazol-5-yl)-8-azabicyclo[3.2.1]octan- |
| | | ((3S,5R)-5-methyl-1-(1H-tetrazol- | | | 2-yl)-1-(4- |
| | | 5-yl)piperidin-3-yl)cyclopropane- | | | (trifluoromethyl)phenyl)cyclopropane- |
| | | 1-carboxamide | | | 1-carboxamide |
| 27A* | 27 | 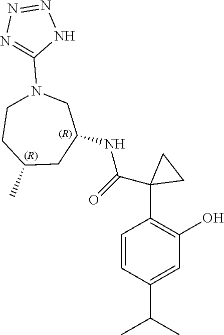 | 79B* | TA | 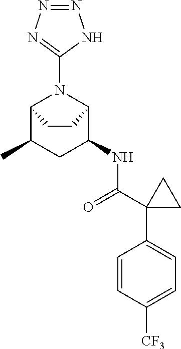 [SMILES: C[C@@H]1C[C@H](NC(=O)C2(c3ccc(C(F)(F)F)cc3)CC2)[C@H]2CC[C@@H]1N2c1nnn[nH]1] |
| | | 1-(2-hydroxy-4-isopropylphenyl)- | | | N-((1R,2S,4R,5S)-4-methyl-8-(1H- |
| | | N-((3R,5R)-5-methyl-1-(1H- | | | tetrazol-5-yl)-8-azabicyclo[3.2.1]octan- |
| | | tetrazol-5-yl)azepan-3- | | | 2-yl)-1-(4- |
| | | yl)cyclopropane-1-carboxamide | | | (trifluoromethyl)phenyl)cyclopropane-1- |
| | | | | | carboxamide |
| 27B* | 27 | 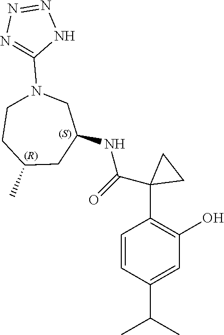 | 79C* | TA | 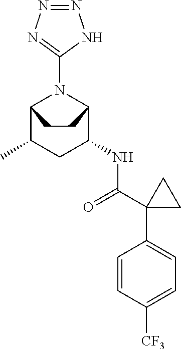 [SMILES: C[C@H]1C[C@@H](NC(=O)C2(c3ccc(C(F)(F)F)cc3)CC2)[C@@H]2CC[C@H]1N2c1nnn[nH]1] |
| | | 1-(2-hydroxy-4-isopropylphenyl)- | | | N-((1S,2R,4S,5R)-4-methyl-8-(1H- |
| | | N-((3S,5R)-5-methyl-1-(1H- | | | tetrazol-5-yl)-8-azabicyclo[3.2.1]octan- |
| | | tetrazol-5-yl)azepan-3- | | | 2-yl)-1-(4- |
| | | yl)cyclopropane-1-carboxamide | | | (trifluoromethyl)phenyl)cyclopropane-1- |
| | | | | | carboxamide |
| 27C* | 27 | 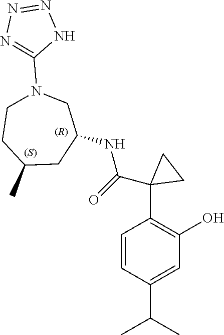 | 79D* | TA | 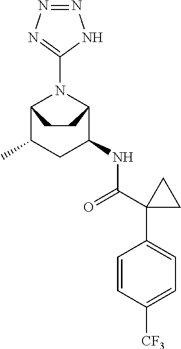 [SMILES: C[C@H]1C[C@H](NC(=O)C2(c3ccc(C(F)(F)F)cc3)CC2)[C@@H]2CC[C@H]1N2c1nnn[nH]1] |
| | | 1-(2-hydroxy-4-isopropylphenyl)- | | | N-((1S,2S,4S,5R)-4-methyl-8-(1H- |
| | | N-((3R,5S)-5-methyl-1-(1H- | | | tetrazol-5-yl)-8-azabicyclo[3.2.1]octan- |
| | | tetrazol-5-yl)azepan-3- | | | 2-yl)-1-(4- |
| | | yl)cyclopropane-1-carboxamide | | | (trifluoromethyl)phenyl)cyclopropane-1- |
| | | | | | carboxamide |
| 27D* | 27 | 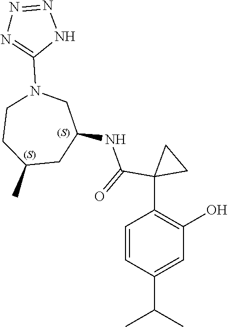 | Rac-80 | TA | 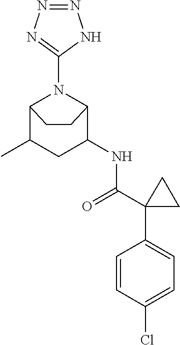 [SMILES: CC1CC(NC(=O)C2(c3ccc(Cl)cc3)CC2)C2CCC1N2c1nnn[nH]1] |
| | | 1-(2-hydroxy-4-isopropylphenyl)- | | | 1-(4-chlorophenyl)-N-(4-methyl-8-(1H- |
| | | N-((3S,5S)-5-methyl-1-(1H- | | | tetrazol-5-yl)-8-azabicyclo[3.2.1]octan- |
| | | tetrazol-5-yl)azepan-3- | | | 2-yl)cyclopropane-1-carboxamide |
| | | yl)cyclopropane-1-carboxamide | | | |
| 27A- OMe* | 27 | 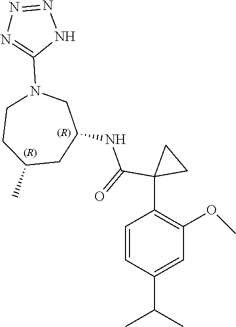 | 80′* | TA | 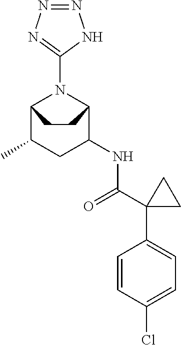 [SMILES: C[C@H]1CC(NC(=O)C2(c3ccc(Cl)cc3)CC2)[C@@H]2CC[C@H]1N2c1nnn[nH]1] |
| | | 1-(4-isopropyl-2-methoxyphenyl)- | | | N-((1R,2R,5S)-4-methyl-8-(1H-tetrazol- |
| | | N-((3R,5R)-5-methyl-1-(1H- | | | 5-yl)-8-azabicyclo[3.2.1]octan-2-yl)-1- |
| | | tetrazol-5-yl)azepan-3- | | | (4-chlorophenyl)cyclopropane-1- |
| | | yl)cyclopropane-1-carboxamide | | | carboxamide |
| 27B- OMe* | 27 | 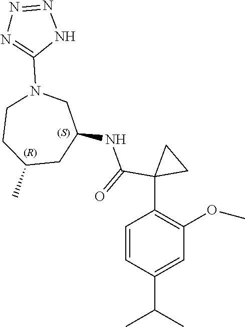 | 80″* | TA | 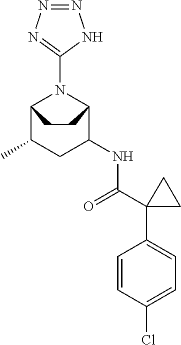 [SMILES: C[C@H]1CC(NC(=O)C2(c3ccc(Cl)cc3)CC2)[C@@H]2CC[C@H]1N2c1nnn[nH]1] |
| | | 1-(4-isopropyl-2-methoxyphenyl)- | | | N-((1S,2S,5R)-4-methyl-8-(1H-tetrazol- |
| | | N-((3S,5R)-5-methyl-1-(1H- | | | 5-yl)-8-azabicyclo[3.2.1]octan-2-yl)-1- |
| | | tetrazol-5-yl)azepan-3- | | | (4-chlorophenyl)cyclopropane-1- |
| | | yl)cyclopropane-1-carboxamide | | | carboxamide |
| 27C- OMe* | 27 | 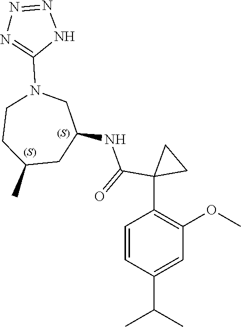 | 80A* | TA | 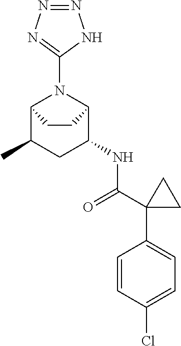 [SMILES: C[C@@H]1C[C@@H](NC(=O)C2(c3ccc(Cl)cc3)CC2)[C@H]2CC[C@@H]1N2c1nnn[nH]1] |
| | | 1-(4-isopropyl-2-methoxyphenyl)- | | | 1-(4-chlorophenyl)-N-((1R,2R,4R,5S)-4- |
| | | N-((3R,5S)-5-methyl-1-(1H- | | | methyl-8-(1H-tetrazol-5-yl)-8- |
| | | tetrazol-5-yl)azepan-3- | | | azabicyclo[3.2.1]octan-2- |
| | | yl)cyclopropane-1-carboxamide | | | yl)cyclopropane-1-carboxamide |
| 27D- OMe* | 27 | 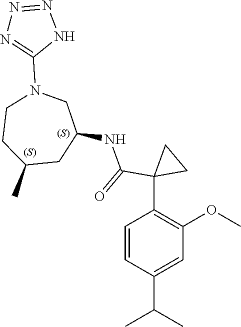 | 80B* | TA | 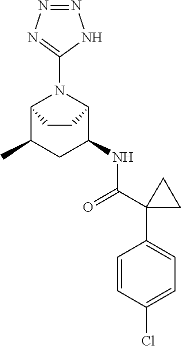 [SMILES: C[C@@H]1C[C@H](NC(=O)C2(c3ccc(Cl)cc3)CC2)[C@H]2CC[C@@H]1N2c1nnn[nH]1] |
| | | 1-(4-isopropyl-2-methoxyphenyl)- | | | 1-(4-chlorophenyl)-N-((1R,2S,4R,5S)-4- |
| | | N-((3S,5S)-5-methyl-1-(1H- | | | methyl-8-(1H-tetrazol-5-yl)-8- |
| | | tetrazol-5-yl)azepan-3- | | | azabicyclo[3.2.1 ]octan-2- |
| | | yl)cyclopropane-1-carboxamide | | | yl)cyclopropane-1-carboxamide |
| 28A* | 28 | 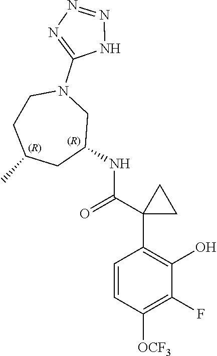 | 80C* | TA | 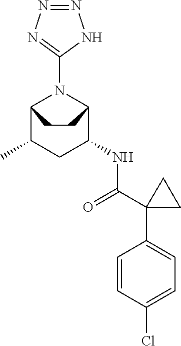 [SMILES: C[C@H]1C[C@@H](NC(=O)C2(c3ccc(Cl)cc3)CC2)[C@@H]2CC[C@H]1N2c1nnn[nH]1] |
| | | 1-(3-fluoro-2-hydroxy-4- | | | 1-(4-chlorophenyl)-N-((1S,2R,4S,5R)-4- |
| | | (trifluoromethoxy)phenyl)-N- | | | methyl-8-(1H-tetrazol-5-yl)-8- |
| | | ((3R,5R)-5-methyl-1-(1H-tetrazol- | | | azabicyclo[3.2.1]octan-2- |
| | | 5-yl)azepan-3-yl)cyclopropane-1- | | | yl)cyclopropane-1-carboxamide |
| | | carboxamide | | | |
| 28B* | 28 | 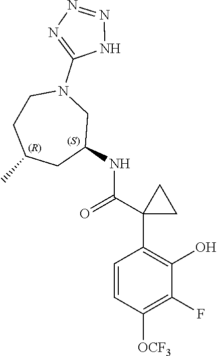 | 80D* | TA | 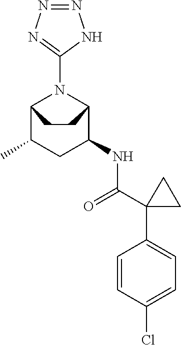 [SMILES: C[C@H]1C[C@H](NC(=O)C2(c3ccc(Cl)cc3)CC2)[C@@H]2CC[C@H]1N2c1nnn[nH]1] |
| | | 1-(3-fluoro-2-hydroxy-4- | | | 1-(4-chlorophenyl)-N-((1S,2S,4S,5R)-4- |
| | | (trifluoromethoxy)phenyl)-N- | | | methyl-8-(1H-tetrazol-5-yl)-8- |
| | | ((3S,5R)-5-methyl-1-(1H-tetrazol- | | | azabicyclo[3.2.1]octan-2- |
| | | 5-yl)azepan-3-yl)cyclopropane-1- | | | yl)cyclopropane-1-carboxamide |
| | | carboxamide | | | |
| 28C* | 28 | 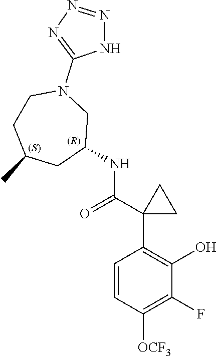 | Rac-81 | TA | 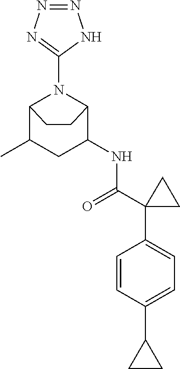 [SMILES: CC1CC(NC(=O)C2(c3ccc(C4CC4)cc3)CC2)C2CCC1N2c1nnn[nH]1] |
| | | 1-(3-fluoro-2-hydroxy-4- | | | 1-(4-cyclopropylphenyl)-N-(4-methyl-8- |
| | | (trifluoromethoxy)phenyl)-N- | | | (1H-tetrazol-5-yl)-8- |
| | | ((3R,5S)-5-methyl-1-(1H-tetrazol- | | | azabicyclo[3.2.1]octan-2- |
| | | 5-yl)azepan-3-yl)cyclopropane-1- | | | yl)cyclopropane-1-carboxamide |
| | | carboxamide | | | |
| 28D* | 28 | 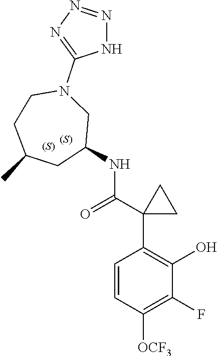 | 81′* | TA | 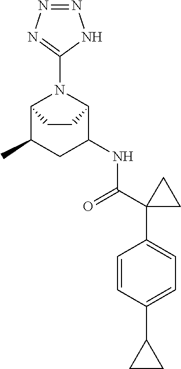 [SMILES: C[C@@H]1CC(NC(=O)C2(c3ccc(C4CC4)cc3)CC2)[C@H]2CC[C@@H]1N2c1nnn[nH]1] |
| | | 1-(3-fluoro-2-hydroxy-4- | | | N-((1R,2R,5S)-4-methyl-8-(1H-tetrazol- |
| | | (trifluoromethoxy)phenyl)-N- | | | 5-yl)-8-azabicyclo[3.2.1]octan-2-yl)-1- |
| | | ((3S,5S)-5-methyl-1-(1H-tetrazol- | | | (4-( cyclopropyl)phenyl)cyclopropane- |
| | | 5-yl)azepan-3-yl)cyclopropane-1- | | | 1-carboxamide |
| | | carboxamide | | | |
| 28A- OMe * | 28 | 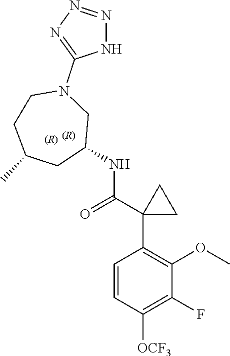 | 81″* | TA | 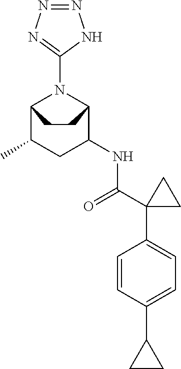 [SMILES: C[C@H]1CC(NC(=O)C2(c3ccc(C4CC4)cc3)CC2)[C@@H]2CC[C@H]1N2c1nnn[nH]1] |
| | | 1-(3-fluoro-2-methoxy-4- | | | N-((1S,2S,5R)-4-methyl-8-(1H-tetrazol- |
| | | (trifluoromethoxy)phenyl)-N- | | | 5-yl)-8-azabicyclo[3.2.1]octan-2-yl)-1- |
| | | ((3R,5R)-5-methyl-1-(1H-tetrazol- | | | (4-( cyclopropyl)phenyl)cyclopropane |
| | | 5-yl)azepan-3-yl)cyclopropane-1- | | | NH |
| | | carboxamide | | | |
| 28B- OMe * | 28 | 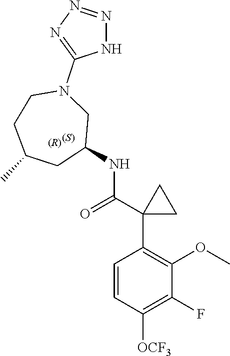 | 81A* | TA | 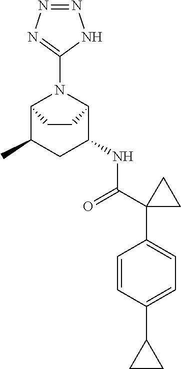 [SMILES: C[C@@H]1C[C@@H](NC(=O)C2(c3ccc(C4CC4)cc3)CC2)[C@H]2CC[C@@H]1N2c1nnn[nH]1] |
| | | 1-(3-fluoro-2-methoxy-4- | | | 1-(4-cyclopropylphenyl)-N- |
| | | (trifluoromethoxy)phenyl)-N- | | | ((1R,2R,4R,5S)-4-methyl-8-(1H- |
| | | ((3S,5R)-5-methyl-1-(1H-tetrazol- | | | tetrazol-5-yl)-8-azabicyclo[3.2.1]octan- |
| | | 5-yl)azepan-3-yl)cyclopropane-1- | | | 2-yl)cyclopropane-1-carboxamide |
| | | carboxamide | | | |
| 28C- OMe * | 28 | 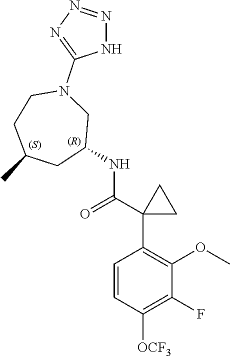 | 81B* | TA | 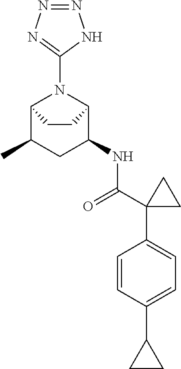 [SMILES: C[C@@H]1C[C@H](NC(=O)C2(c3ccc(C4CC4)cc3)CC2)[C@H]2CC[C@@H]1N2c1nnn[nH]1] |
| | | OCF3 1-(3-fluoro-2-methoxy-4- | | | 1-(4-cyclopropylphenyl)-N- |
| | | (trifluoromethoxy)phenyl)-N- | | | ((1R,2S,4R,5S)-4-methyl-8-(1H- |
| | | ((3R,5S)-5-methyl-1-(1H-tetrazol- | | | tetrazol-5-yl)-8-azabicyclo[3.2.1]octan- |
| | | 5-yl)azepan-3-yl)cyclopropane-1- | | | 2-yl)cyclopropane-1-carboxamide |
| | | carboxamide | | | |
| 28D- OMe * | 28 | 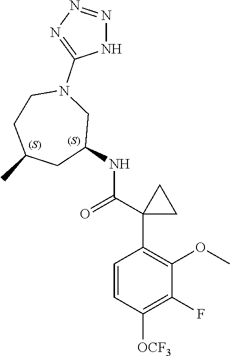 | 81C* | TA | 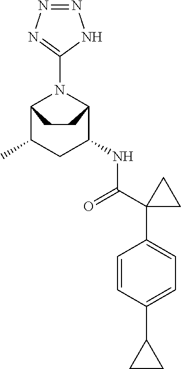 [SMILES: C[C@H]1C[C@@H](NC(=O)C2(c3ccc(C4CC4)cc3)CC2)[C@@H]2CC[C@H]1N2c1nnn[nH]1] |
| | | 1-(3-fluoro-2-methoxy-4- | | | 1-(4-cyclopropylphenyl)-N- |
| | | (trifluoromethoxy)phenyl)-N- | | | ((1S,2R,4S,5R)-4-methyl-8-(1H- |
| | | ((3S,5S)-5-methyl-1-(1H-tetrazol- | | | tetrazol-5-yl)-8-azabicyclo[3.2.1]octan- |
| | | 5-yl)azepan-3-yl)cyclopropane-1- | | | 2-yl)cyclopropane-1-carboxamide |
| | | carboxamide | | | |
| 29A* | 29 | 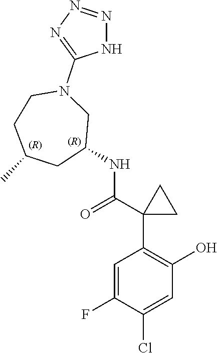 | 81D* | TA | 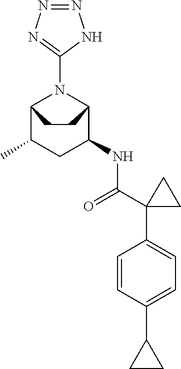 [SMILES: C[C@H]1C[C@H](NC(=O)C2(c3ccc(C4CC4)cc3)CC2)[C@@H]2CC[C@H]1N2c1nnn[nH]1] |
| | | 1-(4-chloro-5-fluoro-2- | | | 1-(4-cyclopropyl-3-fluoro-2- |
| | | hydroxyphenyl)-N-((3R,5R)-5- | | | hydroxyphenyl)-N-((1S,2S,4S,5R)-4- |
| | | methyl-1-(1H-tetrazol-5-yl)azepan- | | | methyl-8-(1H-tetrazol-5-yl)-8- |
| | | 3-yl)cyclopropane-1-carboxamide | | | azabicyclo [3.2.1 ]octan-2- |
| | | | | | yl)cyclopropane-1-carboxamide |
| 29B* | 29 | 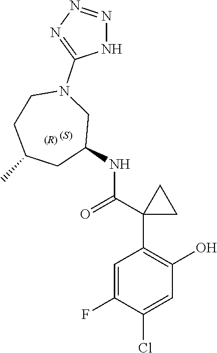 | Rac-82 | TA | 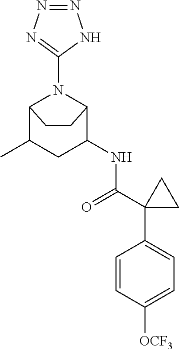 [SMILES: CC1CC(NC(=O)C2(c3ccc(OC(F)(F)F)cc3)CC2)C2CCC1N2c1nnn[nH]1] |
| | | 1-(4-chloro-5-fluoro-2- | | | N-(4-methyl-8-(1H-tetrazol-5-yl)-8- |
| | | hydroxyphenyl)-N-((3S,5R)-5- | | | azabicyclo[3.2.1]octan-2-yl)-1-(4- |
| | | methyl-1-(1H-tetrazol-5-yl)azepan- | | | (trifluoromethoxy)phenyl)cyclopropane- |
| | | 3-yl)cyclopropane-1-carboxamide | | | 1-carboxamide |
| 29C* | 29 | 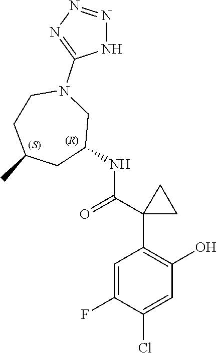 | 82′* | TA | 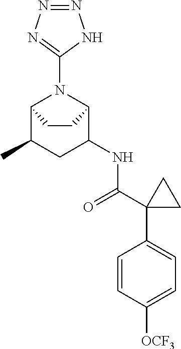 [SMILES: C[C@@H]1CC(NC(=O)C2(c3ccc(OC(F)(F)F)cc3)CC2)[C@H]2CC[C@@H]1N2c1nnn[nH]1] |
| | | 1-(4-chloro-5-fluoro-2- | | | N-((1R,2R,5S)-4-methyl-8-(1H-tetrazol- |
| | | hydroxyphenyl)-N-((3R,5S)-5- | | | 5-yl)-8-azabicyclo[3.2.1]octan-2-yl)-1- |
| | | methyl-1-(1H-tetrazol-5-yl)azepan- | | | (4-(trifluoromethoxy) |
| | | 3-yl)cyclopropane-1-carboxamide | | | phenyl)cyclopropane- |
| 29D* | 29 | 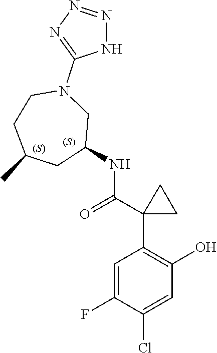 | 82″* | TA | 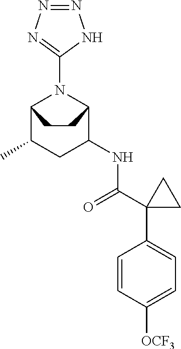 [SMILES: C[C@H]1CC(NC(=O)C2(c3ccc(OC(F)(F)F)cc3)CC2)[C@@H]2CC[C@H]1N2c1nnn[nH]1] |
| | | 1-(4-chloro-5-fluoro-2- | | | N-((1S,2S,5R)-4-methyl-8-(1H-tetrazol- |
| | | hydroxyphenyl)-N-((3S,5S)-5- | | | 5-yl)-8-azabicyclo[3.2.1]octan-2-yl)-1- |
| | | methyl-1-(1H-tetrazol-5-yl)azepan- | | | (4-(trifluoromethoxy) |
| | | 3-yl)cyclopropane-1-carboxamide | | | phenyl)cyclopropane- |
| | | | | | 1-carboxamide |
| 29A- OMe * | 29 | 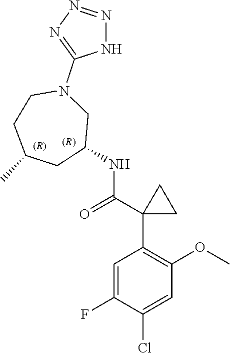 | 82A* | TA | 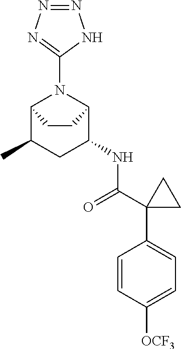 [SMILES: C[C@@H]1C[C@@H](NC(=O)C2(c3ccc(OC(F)(F)F)cc3)CC2)[C@H]2CC[C@@H]1N2c1nnn[nH]1] |
| | | 1-(4-chloro-5-fluoro-2- | | | N-((1R,2R,4R,5S)-4-methyl-8-(1H- |
| | | methoxyphenyl)-N-((3R,5R)-5- | | | tetrazol-5-yl)-8-azabicyclo[3.2.1]octan- |
| | | methyl-1-(1H-tetrazol-5-yl)azepan- | | | 2-yl)-1-(4- |
| | | 3-yl)cyclopropane-1-carboxamide | | | (trifluoromethoxy)phenyl)cyclopropane- |
| | | | | | 1-carboxamide |
| 29B- OMe * | 29 | 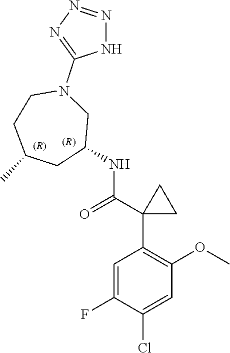 | 82B* | TA | 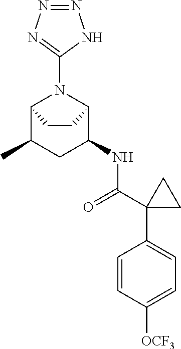 [SMILES: C[C@@H]1C[C@H](NC(=O)C2(c3ccc(OC(F)(F)F)cc3)CC2)[C@H]2CC[C@@H]1N2c1nnn[nH]1] |
| | | 1-(4-chloro-5-fluoro-2- | | | N-((1R,2S,4R,5S)-4-methyl-8-(1H- |
| | | methoxyphenyl)-N-((3S,5R)-5- | | | tetrazol-5-yl)-8-azabicyclo[3.2.1]octan- |
| | | methyl-1-(1H-tetrazol-5-yl)azepan- | | | 2-yl)-1-(4- |
| | | 3-yl)cyclopropane-1-carboxamide | | | (trifluoromethoxy)phenyl)cyclopropane- |
| | | | | | 1-carboxamide |
| 29C- OMe * | 29 | 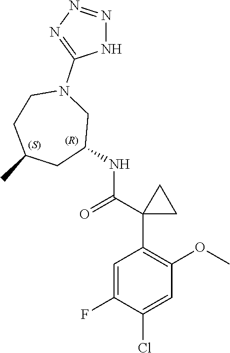 | 82C* | TA | 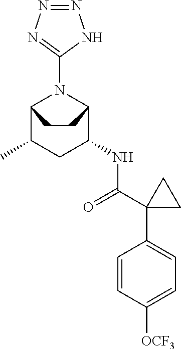 [SMILES: C[C@H]1C[C@@H](NC(=O)C2(c3ccc(OC(F)(F)F)cc3)CC2)[C@@H]2CC[C@H]1N2c1nnn[nH]1] |
| | | 1-(4-chloro-5-fluoro-2- | | | N-((1S,2R,4S,5R)-4-methyl-8-(1H- |
| | | methoxyphenyl)-N-((3R,5S)-5- | | | tetrazol-5-yl)-8-azabicyclo[3.2.1]octan- |
| | | methyl-1-(1H-tetrazol-5-yl)azepan- | | | 2-yl)-1-(4- |
| | | 3-yl)cyclopropane-1-carboxamide | | | (trifluoromethoxy)phenyl)cyclopropane- |
| | | | | | 1-carboxamide |
| 29D- OMe * | 29 | 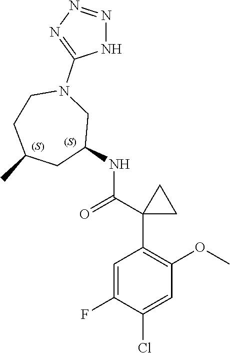 | 82D* | TA | 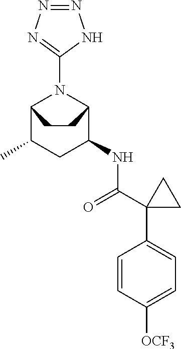 [SMILES: C[C@H]1C[C@H](NC(=O)C2(c3ccc(OC(F)(F)F)cc3)CC2)[C@@H]2CC[C@H]1N2c1nnn[nH]1] |
| | | 1-(4-chloro-5-fluoro-2- | | | N-((1S,2S,4S,5R)-4-methyl-8-(1H- |
| | | methoxyphenyl)-N-((3S,5S)-5- | | | tetrazol-5-yl)-8-azabicyclo[3.2.1]octan- |
| | | methyl-1-(1H-tetrazol-5-yl)azepan- | | | 2-yl)-1-(4- |
| | | 3-yl)cyclopropane-1-carboxamide | | | (trifluoromethoxy)phenyl)cyclopropane- |
| | | | | | 1-carboxamide |
| 30A* | 30 | 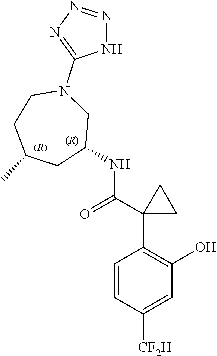 | Rac-83 | TA | 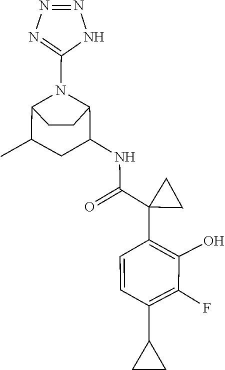 [SMILES: CC1CC(NC(=O)C2(c3ccc(C4CC4)c(F)c3O)CC2)C2CCC1N2c1nnn[nH]1] |
| | | 1-(4-(difluoromethyl)-2- | | | 1-(4-cyclopropyl-3-fluoro-2- |
| | | hydroxyphenyl)-N-((3R,5R)-5- | | | hydroxyphenyl)-N-(4-methyl-8-(1H- |
| | | methyl-1-(1H-tetrazol-5-yl)azepan- | | | tetrazol-5-yl)-8-azabicyclo[3.2.1]octan- |
| | | 3-yl)cyclopropane-1-carboxamide | | | 2-yl)cyclopropane-1-carboxamide |
| 30B* | 30 | 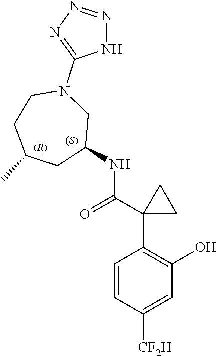 | 83′* | TA | 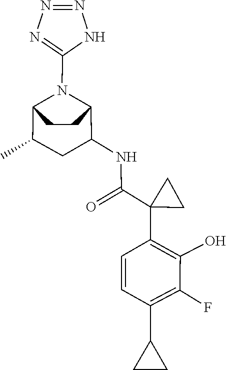 [SMILES: C[C@H]1CC(NC(=O)C2(c3ccc(C4CC4)c(F)c3O)CC2)[C@@H]2CC[C@H]1N2c1nnn[nH]1] |
| | | 1-(4-(difluoromethyl)-2- | | | N-((1R,2R,5S)-4-methyl-8-(1H-tetrazol- |
| | | hydroxyphenyl)-N-((3S,5R)-5- | | | 5-yl)-8-azabicyclo[3.2.1]octan-2-yl)-1- |
| | | methyl-1-(1H-tetrazol-5-yl)azepan- | | | (4-cyclopropyl-3-fluoro-2- |
| | | 3-yl)cyclopropane-1-carboxamide | | | hydroxyphenyl)cyclopropane-1 |
| 30C* | 30 | 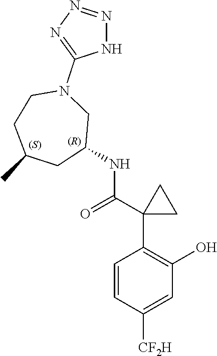 | 83″* | TA | 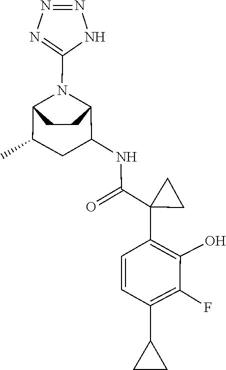 [SMILES: C[C@H]1CC(NC(=O)C2(c3ccc(C4CC4)c(F)c3O)CC2)[C@@H]2CC[C@H]1N2c1nnn[nH]1] |
| | | 1-(4-(difluoromethyl)-2- | | | N-((1S,2S,5R)-4-methyl-8-(1H-tetrazol- |
| | | hydroxyphenyl)-N-((3R,5S)-5- | | | 5-yl)-8-azabicyclo[3.2.1]octan-2-yl)-1- |
| | | methyl-1-(1H-tetrazol-5-yl)azepan- | | | (4-cyclopropyl-3-fluoro-2- |
| | | 3-yl)cyclopropane-1-carboxamide | | | hydroxyphenyl)cyclopropane |
| 30D* | 30 | 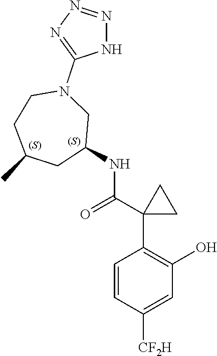 | 83A* | TA | 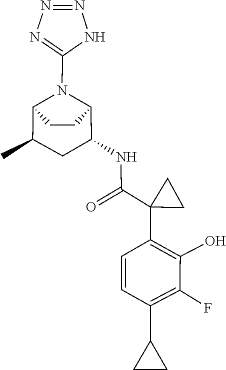 [SMILES: C[C@@H]1C[C@@H](NC(=O)C2(c3ccc(C4CC4)c(F)c3O)CC2)[C@H]2CC[C@@H]1N2c1nnn[nH]1] |
| | | 1-(4-(difluoromethyl)-2- | | | 1-(4-cyclopropyl-3-fluoro-2- |
| | | hydroxyphenyl)-N-((3S,5S)-5- | | | hydroxyphenyl)-N-((1R,2R,4R,5S)-4- |
| | | methyl-1-(1H-tetrazol-5-yl)azepan- | | | methyl-8-(1H-tetrazol-5-yl)-8- |
| | | 3-yl)cyclopropane-1-carboxamide | | | azabicyclo[3.2.1]octan-2- |
| | | | | | yl)cyclopropane-1-carboxamide |
| 30A- OMe * | 30 | 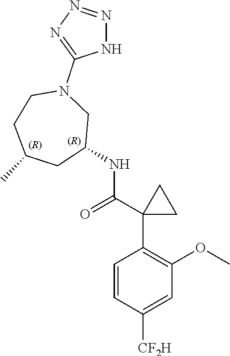 | 83B* | TA | 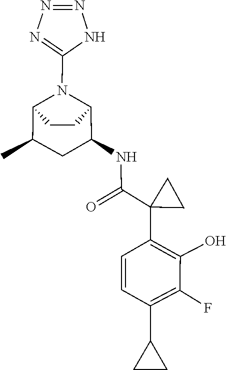 [SMILES: C[C@@H]1C[C@H](NC(=O)C2(c3ccc(C4CC4)c(F)c3O)CC2)[C@H]2CC[C@@H]1N2c1nnn[nH]1] |
| | | 1-(4-(difluoromethyl)-2- | | | 1-(4-cyclopropyl-3-fluoro-2- |
| | | methoxyphenyl)-N-((3R,5R)-5- | | | hydroxyphenyl)-N-((1R,2S,4R,5S)-4- |
| | | methyl-1-(1H-tetrazol-5-yl)azepan- | | | methyl-8-(1H-tetrazol-5-yl)-8- |
| | | 3-yl)cyclopropane-1-carboxamide | | | azabicyclo[3.2.1]octan-2- |
| | | | | | yl)cyclopropane-1-carboxamide |
| 30B- OMe * | 30 | 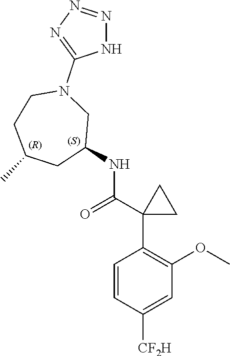 | 83C* | TA | 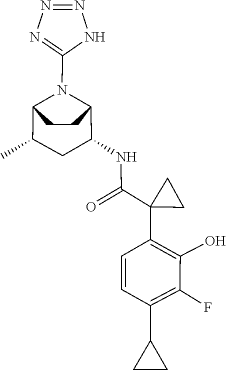 [SMILES: C[C@H]1C[C@@H](NC(=O)C2(c3ccc(C4CC4)c(F)c3O)CC2)[C@@H]2CC[C@H]1N2c1nnn[nH]1] |
| | | 1-(4-(difluoromethyl)-2- | | | 1-(4-cyclopropyl-3-fluoro-2- |
| | | methoxyphenyl)-N-((3S,5R)-5- | | | hydroxyphenyl)-N-((1S,2R,4S,5R)-4- |
| | | methyl-1-(1H-tetrazol-5-yl)azepan- | | | methyl-8-(1H-tetrazol-5-yl)-8- |
| | | 3-yl)cyclopropane-1-carboxamide | | | azabicyclo [3.2.1 ]octan-2- |
| | | | | | yl)cyclopropane-1-carboxamide |
| 30C- OMe * | 30 | 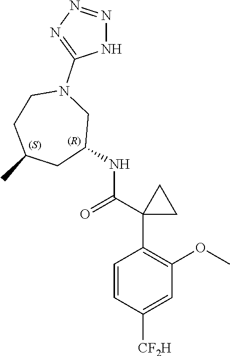 | 83D* | TA | 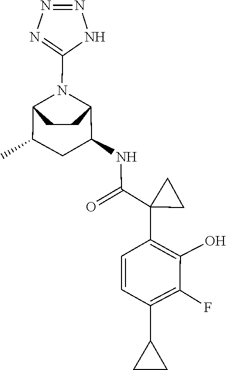 [SMILES: C[C@H]1C[C@H](NC(=O)C2(c3ccc(C4CC4)c(F)c3O)CC2)[C@@H]2CC[C@H]1N2c1nnn[nH]1] |
| | | 1-(4-(difluoromethyl)-2- | | | 1-(4-cyclopropyl-3-fluoro-2- |
| | | methoxyphenyl)-N-((3R,5S)-5- | | | hydroxyphenyl)-N-((1S,2S,4S,5R)-4- |
| | | methyl-1-(1H-tetrazol-5-yl)azepan- | | | methyl-8-(1H-tetrazol-5-yl)-8- |
| | | 3-yl)cyclopropane-1-carboxamide | | | azabicyclo[3.2.1]octan-2- |
| | | | | | yl)cyclopropane-1-carboxamide |
| 30D- OMe * | 30 | 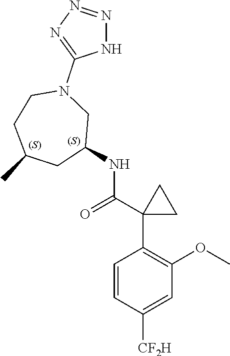 | Rac-84 | TA | 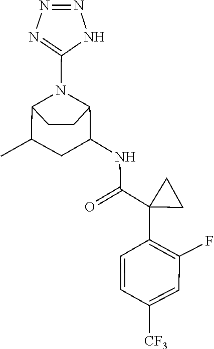 [SMILES: CC1CC(NC(=O)C2(c3ccc(C(F)(F)F)cc3F)CC2)C2CCC1N2c1nnn[nH]1] |
| | | 1-(4-(difluoromethyl)-2- | | | 1-(2-fluoro-4-(trifluoromethyl)phenyl)- |
| | | methoxyphenyl)-N-((3S,5S)-5- | | | N-(4-methyl-8-(1H-tetrazol-5-yl)-8- |
| | | methyl-1-(1H-tetrazol-5-yl)azepan- | | | azabicyclo[3.2.1]octan-2- |
| | | 3-yl)cyclopropane-1-carboxamide | | | yl)cyclopropane-1-carboxamide |
| 31A* | 31 | 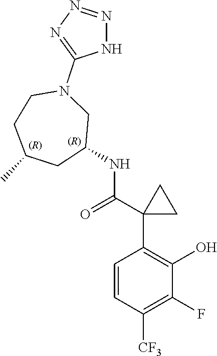 | 84′* | TA | 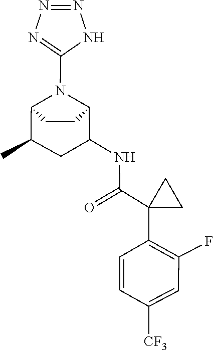 [SMILES: C[C@@H]1CC(NC(=O)C2(c3ccc(C(F)(F)F)cc3F)CC2)[C@H]2CC[C@@H]1N2c1nnn[nH]1] |
| | | 1-(3-fluoro-2-hydroxy-4- | | | N-((1R,2R,5S)-4-methyl-8-(1H-tetrazol- |
| | | (trifluoromethyl)phenyl)-N- | | | 5-yl)-8-azabicyclo[3.2.1]octan-2-yl)-1- |
| | | ((3R,5R)-5-methyl-1-(1H-tetrazol- | | | (2-fluoro-4- |
| | | 5-yl)azepan-3-yl)cyclopropane-1- | | | (trifluoromethyl)phenyl)cyclopropane-1- |
| | | carboxamide | | | carboxamide yl)cyclopropane-1- |
| | | | | | carboxamide |
| 31B* | 31 | 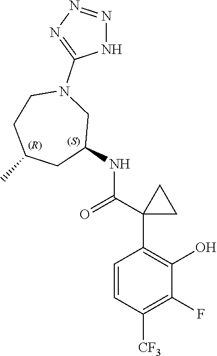 | 84″* | TA | 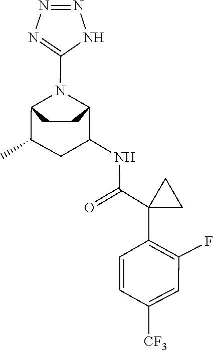 [SMILES: C[C@H]1CC(NC(=O)C2(c3ccc(C(F)(F)F)cc3F)CC2)[C@@H]2CC[C@H]1N2c1nnn[nH]1] |
| | | 1-(3-fluoro-2-hydroxy-4- | | | N-((1S,2S,5R)-4-methyl-8-(1H-tetrazol- |
| | | (trifluoromethyl)phenyl)-N- | | | 5-yl)-8-azabicyclo[3.2.1]octan-2-yl)-1- |
| | | ((3S,5R)-5-methyl-1-(1H-tetrazol- | | | (2-fluoro-4- |
| | | 5-yl)azepan-3-yl)cyclopropane-1- | | | (trifluoromethyl)phenyl)cyclopropane-1- |
| | | carboxamide | | | carboxamide |
| 31C* | 31 | 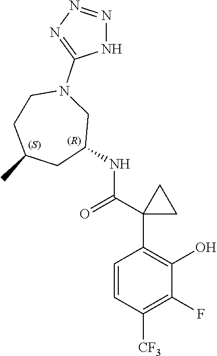 | 84A* | TA | 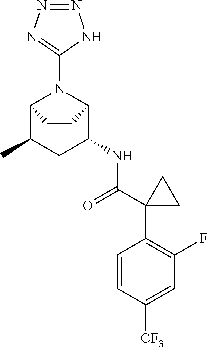 [SMILES: C[C@@H]1C[C@@H](NC(=O)C2(c3ccc(C(F)(F)F)cc3F)CC2)[C@H]2CC[C@@H]1N2c1nnn[nH]1] |
| | | 1-(3-fluoro-2-hydroxy-4- | | | 1-(2-fluoro-4-(trifluoromethyl)phenyl)- |
| | | (trifluoromethyl)phenyl)-N- | | | N-((1R,2R,4R,5S)-4-methyl-8-(1H- |
| | | ((3R,5S)-5-methyl-1-(1H-tetrazol- | | | tetrazol-5-yl)-8-azabicyclo[3.2.1]octan- |
| | | 5-yl)azepan-3-yl)cyclopropane-1- | | | 2-yl)cyclopropane-1-carboxamide |
| | | carboxamide | | | |
| 31D* | 31 | 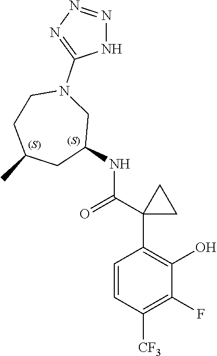 | 84B* | TA | 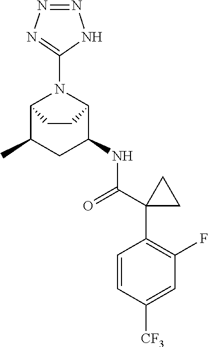 [SMILES: C[C@@H]1C[C@H](NC(=O)C2(c3ccc(C(F)(F)F)cc3F)CC2)[C@H]2CC[C@@H]1N2c1nnn[nH]1] |
| | | 1-(3-fluoro-2-hydroxy-4- | | | 1-(2-fluoro-4-(trifluoromethyl)phenyl)- |
| | | (trifluoromethyl)phenyl)-N- | | | N-((1R,2S,4R,5S)-4-methyl-8-(1H- |
| | | ((3S,5S)-5-methyl-1-(1H-tetrazol- | | | tetrazol-5-yl)-8-azabicyclo[3.2.1]octan- |
| | | 5-yl)azepan-3-yl)cyclopropane-1- | | | 2-yl)cyclopropane-1-carboxamide |
| | | carboxamide | | | |
| 31A- OMe * | 31 | 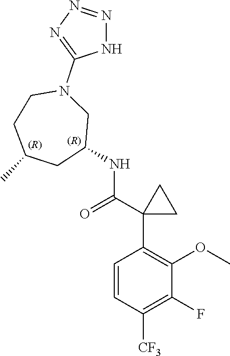 | 84C* | TA | 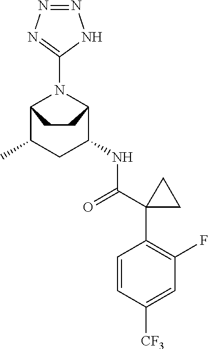 [SMILES: C[C@H]1C[C@@H](NC(=O)C2(c3ccc(C(F)(F)F)cc3F)CC2)[C@@H]2CC[C@H]1N2c1nnn[nH]1] |
| | | 1-(3-fluoro-2-methoxy-4- | | | 1-(2-fluoro-4-(trifluoromethyl)phenyl)- |
| | | (trifluoromethyl)phenyl)-N- | | | N-((1S,2R,4S,5R)-4-methyl-8-(1H- |
| | | ((3R,5R)-5-methyl-1-(1H-tetrazol- | | | tetrazol-5-yl)-8-azabicyclo[3.2.1]octan- |
| | | 5-yl)azepan-3-yl)cyclopropane-1- | | | 2-yl)cyclopropane-1-carboxamide |
| | | carboxamide | | | |
| 31B- OMe * | 31 | 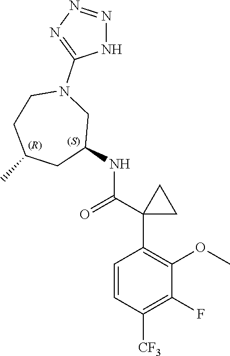 | 84D* | TA | 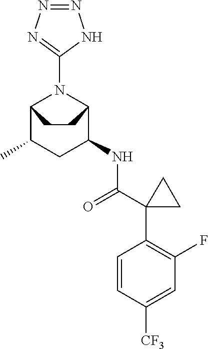 [SMILES: C[C@H]1C[C@H](NC(=O)C2(c3ccc(C(F)(F)F)cc3F)CC2)[C@@H]2CC[C@H]1N2c1nnn[nH]1] |
| | | 1-(3-fluoro-2-methoxy-4- | | | 1-(2-fluoro-4-(trifluoromethyl)phenyl)- |
| | | (trifluoromethyl)phenyl)-N- | | | N-((1S,2S,4S,5R)-4-methyl-8-(1H- |
| | | ((3S,5R)-5-methyl-1-(1H-tetrazol- | | | tetrazol-5-yl)-8-azabicyclo[3.2.1]octan- |
| | | 5-yl)azepan-3-yl)cyclopropane-1- | | | 2-yl)cyclopropane-1-carboxamide |
| | | carboxamide | | | |
| 31C- OMe * | 31 | 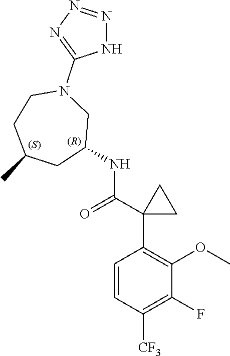 | Rac-85 | TA | 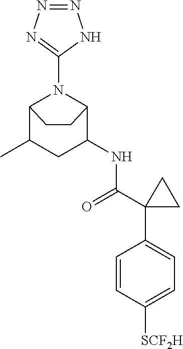 |
| | | 1-(3-fluoro-2-methoxy-4- | | | 1-(4-((difluoromethyl)thio)phenyl)-N-(4- |
| | | (trifluoromethyl)phenyl)-N- | | | methyl-8-(1H-tetrazol-5-yl)-8- |
| | | ((3R,5S)-5-methyl-1-(1H-tetrazol- | | | azabicyclo[3.2.1]octan-2- |
| | | 5-yl)azepan-3-yl)cyclopropane-1- | | | yl)cyclopropane-1-carboxamide |
| | | carboxamide | | | |
| 31D- OMe * | 31 | 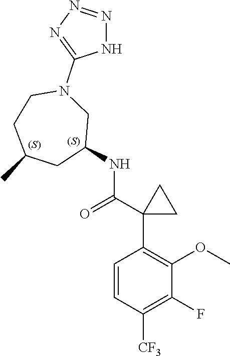 | 85′* | TA | 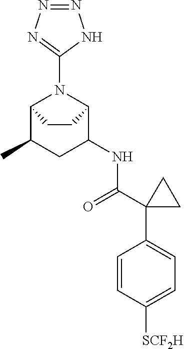 |
| | | 1-(3-fluoro-2-methoxy-4- | | | N-((1R,2R,5S)-4-methyl-8-(1H-tetrazol- |
| | | (trifluoromethyl)phenyl)-N- | | | 5-yl)-8-azabicyclo[3.2.1]octan-2-yl)-1- |
| | | ((3S,5S)-5-methyl-1-(1H-tetrazol- | | | (4-((difluoromethyl) |
| | | 5-yl)azepan-3-yl)cyclopropane-1- | | | thio)phenyl)cyclopropane- |
| | | carboxamide | | | 1-carboxamide |
| 32A* | 32 | 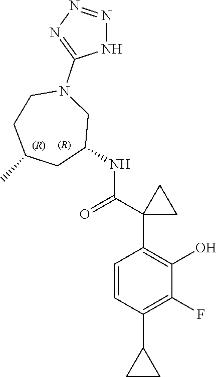 | 85″* | TA | 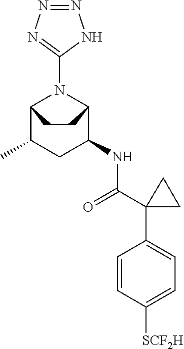 |
| | | 1-(4-cyclopropyl-3-fluoro-2- | | | N-((1S,2S,5R)-4-methyl-8-(1H-tetrazol- |
| | | hydroxyphenyl)-N-((3R,5R)-5- | | | 5-yl)-8-azabicyclo[3.2.1]octan-2-yl)-1- |
| | | methyl-1-(1H-tetrazol-5-yl)azepan- | | | (4-((difluoromethyl) |
| | | 3-yl)cyclopropane-1-carboxamide | | | thio)phenyl)cyclopropane- |
| | | | | | 1-carboxamide |
| 32B* | 32 | 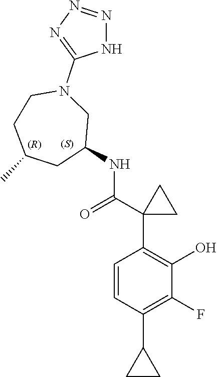 | 85A* | TA | 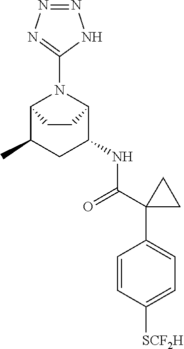 |
| | | 1-(4-cyclopropyl-3-fluoro-2- | | | 1-(4-((difluoromethyl)thio)phenyl)-N- |
| | | hydroxyphenyl)-N-((3S,5R)-5- | | | ((1R,2R,4R,5S)-4-methyl-8-(1H- |
| | | methyl-1-(1H-tetrazol-5-yl)azepan- | | | tetrazol-5-yl)-8-azabicyclo[3.2.1]octan- |
| | | 3-yl)cyclopropane-1-carboxamide | | | 2-yl)cyclopropane-1-carboxamide |
| 32C* | 32 | 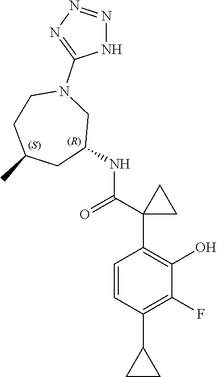 | 85B* | TA | 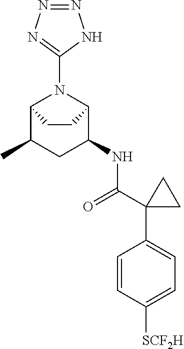 |
| | | 1-(4-cyclopropyl-3-fluoro-2- | | | 1-(4-((difluoromethyl)thio)phenyl)-N- |
| | | hydroxyphenyl)-N-((3R,5S)-5- | | | ((1R,2S,4R,5S)-4-methyl-8-(1H- |
| | | methyl-1-(1H-tetrazol-5-yl)azepan- | | | tetrazol-5-yl)-8-azabicyclo[3.2.1]octan- |
| | | 3-yl)cyclopropane-1-carboxamide | | | 2-yl)cyclopropane-1-carboxamide |
| 32D* | 32 | 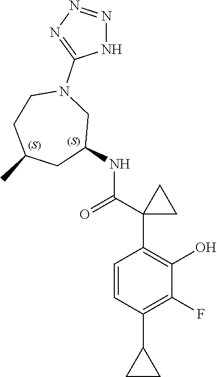 | 85C* | TA | 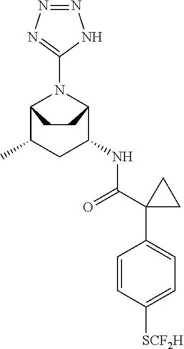 |
| | | 1-(4-cyclopropyl-3-fluoro-2- | | | 1-(4-((difluoromethyl)thio)phenyl)-N- |
| | | hydroxyphenyl)-N-((3S,5S)-5- | | | ((1S,2R,4S,5R)-4-methyl-8-(1H- |
| | | methyl-1-(1H-tetrazol-5-yl)azepan- | | | tetrazol-5-yl)-8-azabicyclo[3.2.1]octan- |
| | | 3-yl)cyclopropane-1-carboxamide | | | 2-yl)cyclopropane-1-carboxamide |
| 32A- OMe * | 32 | 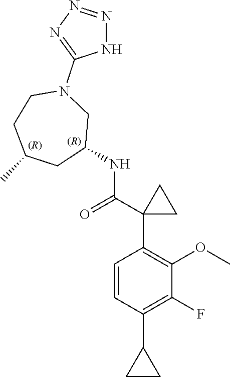 | 85D* | TA | 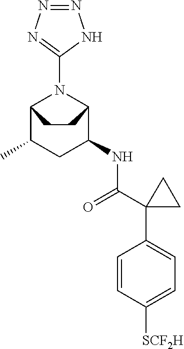 |
| | | 1-(4-cyclopropyl-3-fluoro-2- | | | 1-(4-((difluoromethyl)thio)phenyl)-N- |
| | | methoxyphenyl)-N-((3R,5R)-5- | | | ((1S,2S,4S,5R)-4-methyl-8-(1H-tetrazol- |
| | | methyl-1-(1H-tetrazol-5-yl)azepan- | | | 5-yl)-8-azabicyclo[3.2.1]octan-2- |
| | | 3-yl)cyclopropane-1-carboxamide | | | yl)cyclopropane-1-carboxamide |
| 32B- OMe * | 32 | 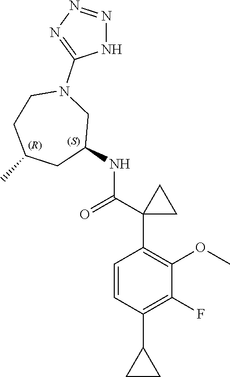 | Rac-86 | TA | 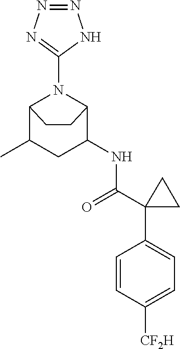 [SMILES: CC1CC(NC(=O)C2(c3ccc(C(F)F)cc3)CC2)C2CCC1N2c1nnn[nH]1] |
| | | 1-(4-cyclopropyl-3-fluoro-2- | | | 1-(4-(difluoromethyl)phenyl)-N-(4- |
| | | methoxyphenyl)-N-((3S,5R)-5- | | | methyl-8-(1H-tetrazol-5-yl)-8- |
| | | methyl-1-(1H-tetrazol-5-yl)azepan- | | | azabicyclo [3.2.1]octan-2- |
| | | 3-yl)cyclopropane-1-carboxamide | | | yl)cyclopropane-1-carboxamide |
| 32C- OMe * | 32 | 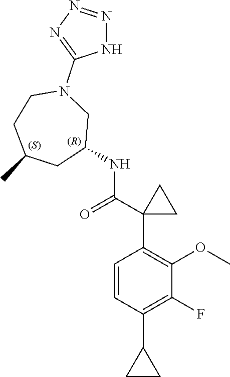 | 86′* | TA | 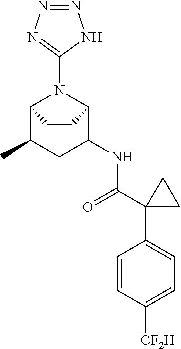 [SMILES: C[C@@H]1CC(NC(=O)C2(c3ccc(C(F)F)cc3)CC2)[C@H]2CC[C@@H]1N2c1nnn[nH]1] |
| | | 1-(4-cyclopropyl-3-fluoro-2- | | | N-((1R,2R,5S)-4-methyl-8-(1H-tetrazol- |
| | | methoxyphenyl)-N-((3R,5S)-5- | | | 5-yl)-8-azabicyclo[3.2.1]octan-2-yl)-1- |
| | | methyl-1-(1H-tetrazol-5-yl)azepan- | | | (4-(difluoromethyl)phenyl)cyclopropane |
| | | 3-yl)cyclopropane-1-carboxamide | | | |
| 32D- OMe * | 32 | 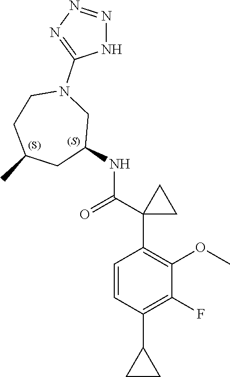 | 86″* | TA | 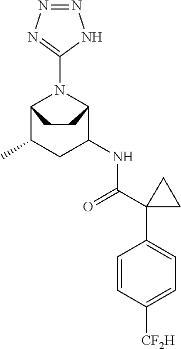 [SMILES: C[C@H]1CC(NC(=O)C2(c3ccc(C(F)F)cc3)CC2)[C@@H]2CC[C@H]1N2c1nnn[nH]1] |
| | | 1-(4-cyclopropyl-3-fluoro-2- | | | N-((1S,2S,5R)-4-methyl-8-(1H-tetrazol- |
| | | methoxyphenyl)-N-((3S,5S)-5- | | | 5-yl)-8-azabicyclo[3.2.1]octan-2-yl)-1- |
| | | methyl-1-(1H-tetrazol-5-yl)azepan- | | | (4-(difluoromethyl) |
| | | 3-yl)cyclopropane-1-carboxamide | | | phenyl)cyclopropane-1- |
| | | | | | carboxamide |
| 33A | 33 | 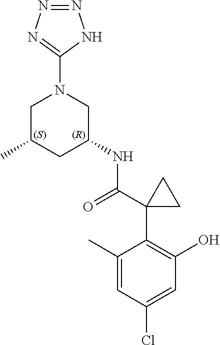 | 86A* | TA | 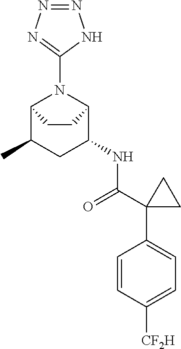 [SMILES: C[C@@H]1C[C@@H](NC(=O)C2(c3ccc(C(F)F)cc3)CC2)[C@H]2CC[C@@H]1N2c1nnn[nH]1] |
| | | 1-(4-chloro-2-hydroxy-6- | | | 1-(4-(difluoromethyl)phenyl)-N- |
| | | methylphenyl)-N-((3R,5S)-5- | | | ((1R,2R,4R,5S)-4-methyl-8-(1H- |
| | | methyl-1-(1H-tetrazol-5- | | | tetrazol-5-yl)-8-azabicyclo[3.2.1]octan- |
| | | yl)piperidin-3-yl)cyclopropane-1- | | | 2-yl)cyclopropane-1-carboxamide |
| | | carboxamide | | | |
| 33B | 33 | 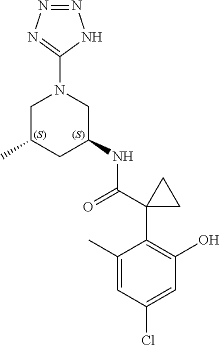 | 86B* | TA | 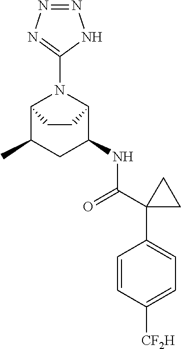 [SMILES: C[C@@H]1C[C@H](NC(=O)C2(c3ccc(C(F)F)cc3)CC2)[C@H]2CC[C@@H]1N2c1nnn[nH]1] |
| | | 1-(4-chloro-2-hydroxy-6- | | | 1-(4-(difluoromethyl)phenyl)-N- |
| | | methylphenyl)-N-((3S,5S)-5- | | | ((1R,2S,4R,5S)-4-methyl-8-(1H- |
| | | methyl-1-(1H-tetrazol-5- | | | tetrazol-5-yl)-8-azabicyclo[3.2.1]octan- |
| | | yl)piperidin-3-yl)cyclopropane-1- | | | 2-yl)cyclopropane-1-carboxamide |
| | | carboxamide | | | |
| 33C | 33 | 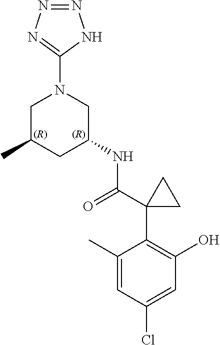 | 86C* | TA | 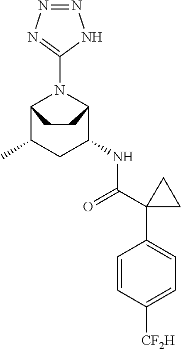 [SMILES: C[C@H]1C[C@@H](NC(=O)C2(c3ccc(C(F)F)cc3)CC2)[C@@H]2CC[C@H]1N2c1nnn[nH]1] |
| | | 1-(4-chloro-2-hydroxy-6- | | | 1-(4-(difluoromethyl)phenyl)-N- |
| | | methylphenyl)-N-((3R,5R)-5- | | | ((1S,2R,4S,5R)-4-methyl-8-(1H- |
| | | methyl-1-(1H-tetrazol-5- | | | tetrazol-5-yl)-8-azabicyclo[3.2.1]octan- |
| | | yl)piperidin-3-yl)cyclopropane-1- | | | 2-yl)cyclopropane-1-carboxamide |
| | | carboxamide | | | |
| 33D | 33 | 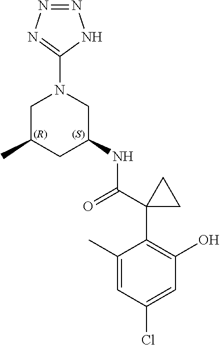 | 86D* | TA | 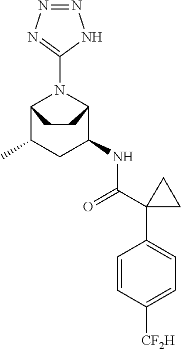 [SMILES: C[C@H]1C[C@H](NC(=O)C2(c3ccc(C(F)F)cc3)CC2)[C@@H]2CC[C@H]1N2c1nnn[nH]1] |
| | | 1-(4-chloro-2-hydroxy-6- | | | 1-(4-(difluoromethyl)phenyl)-N- |
| | | methylphenyl)-N-((3S,5R)-5- | | | ((1S,2S,4S,5R)-4-methyl-8-(1H-tetrazol- |
| | | methyl-1-(1H-tetrazol-5- | | | 5-yl)-8-azabicyclo[3.2.1]octan-2- |
| | | yl)piperidin-3-yl)cyclopropane-1- | | | yl)cyclopropane-1-carboxamide |
| | | carboxamide | | | |
| 33A- OMe | 33 | 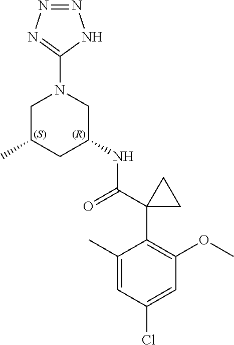 | Rac-87 | TA | 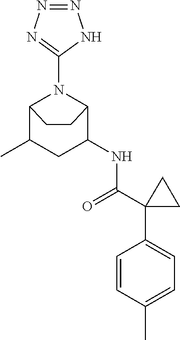 [SMILES: Cc1ccc(C2(C(=O)NC3CC(C)C4CCC3N4c3nnn[nH]3)CC2)cc1] |
| | | 1-(4-chloro-2-methoxy-6- | | | N-(4-methyl-8-(1H-tetrazol-5-yl)-8- |
| | | methylphenyl)-N-((3R,5S)-5- | | | azabicyclo[3.2.1]octan-2-yl)-1-(p- |
| | | methyl-1-(1H-tetrazol-5- | | | tolyl)cyclopropane-1-carboxamide |
| | | yl)piperidin-3-yl)cyclopropane-1- | | | |
| | | carboxamide | | | |
| 33- OMe B | 33 | 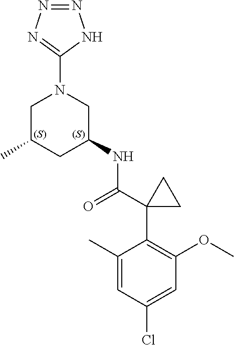 | 87′* | TA | 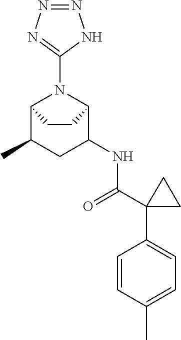 [SMILES: Cc1ccc(C2(C(=O)NC3C[C@@H](C)[C@@H]4CC[C@H]3N4c3nnn[nH]3)CC2)cc1] |
| | | 1-(4-chloro-2-methoxy-6- | | | N-((1R,2R,5S)-4-methyl-8-(1H-tetrazol- |
| | | methylphenyl)-N-((3S,5S)-5- | | | 5-yl)-8-azabicyclo[3.2.1]octan-2-yl)-1- |
| | | methyl-1-(1H-tetrazol-5- | | | (4-tolyl)cyclopropane-1-carboxamide |
| | | yl)piperidin-3-yl)cyclopropane-1- | | | |
| | | carboxamide | | | |
| 33- OMe C | 33 | 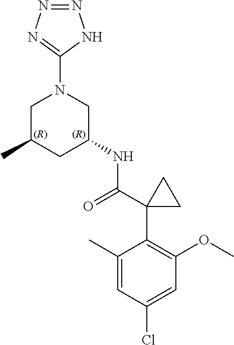 | 87″* | TA | 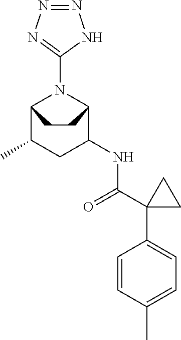 [SMILES: Cc1ccc(C2(C(=O)NC3C[C@H](C)[C@H]4CC[C@@H]3N4c3nnn[nH]3)CC2)cc1] |
| | | 1-(4-chloro-2-methoxy-6- | | | N-((1S,2S,5R)-4-methyl-8-(1H-tetrazol- |
| | | methylphenyl)-N-((3R,5R)-5- | | | 5-yl)-8-azabicyclo[3.2.1]octan-2-yl)-1- |
| | | methyl-1-(1H-tetrazol-5- | | | (4-tolyl)cyclopropane-1-carboxamide |
| | | yl)piperidin-3-yl)cyclopropane-1- | | | |
| | | carboxamide | | | |
| 33- OMe D | 33 | 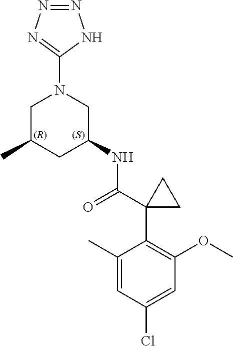 | 87A* | TA | 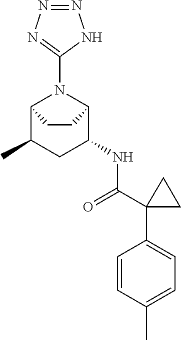 [SMILES: Cc1ccc(C2(C(=O)N[C@@H]3C[C@@H](C)[C@@H]4CC[C@H]3N4c3nnn[nH]3)CC2)cc1] |
| | | 1-(4-chloro-2-methoxy-6- | | | N-((1R,2R,4R,5S)-4-methyl-8-(1H- |
| | | methylphenyl)-N-((3S,5R)-5- | | | tetrazol-5-yl)-8-azabicyclo[3.2.1]octan- |
| | | methyl-1-(1H-tetrazol-5- | | | 2-yl)-1-(p-tolyl)cyclopropane-1- |
| | | yl)piperidin-3-yl)cyclopropane-1- | | | carboxamide |
| | | carboxamide | | | |
| 34A* | 34 | 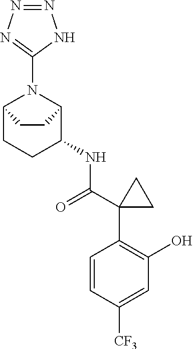 [SMILES: O=C(N[C@@H]1CC[C@@H]2CC[C@H]1N2c1nnn[nH]1)C1(c2ccc(C(F)(F)F)cc2O)CC1] | 87B* | TA | 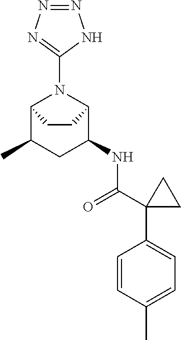 [SMILES: Cc1ccc(C2(C(=O)N[C@H]3C[C@@H](C)[C@@H]4CC[C@H]3N4c3nnn[nH]3)CC2)cc1] |
| | | N-((1R,2R,5R)-8-(1H-tetrazol-5- | | | N-((1R,2S,4R,5S)-4-methyl-8-(1H- |
| | | yl)-8-azabicyclo[3.2.1]octan-2-yl)- | | | tetrazol-5-yl)-8-azabicyclo[3.2.1]octan- |
| | | 1-(2-hydroxy-4- | | | 2-yl)-1-(p-tolyl)cyclopropane-1- |
| | | (trifluoromethyl)phenyl) | | | carboxamide |
| | | cyclopropane-1-carboxamide | | | |
| 34B* | 34 | 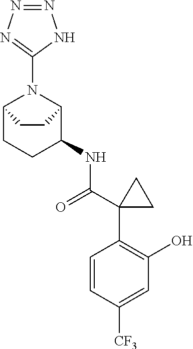 [SMILES: O=C(N[C@H]1CC[C@@H]2CC[C@H]1N2c1nnn[nH]1)C1(c2ccc(C(F)(F)F)cc2O)CC1] | 87C* | TA | 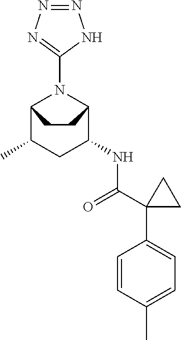 [SMILES: Cc1ccc(C2(C(=O)N[C@@H]3C[C@H](C)[C@H]4CC[C@@H]3N4c3nnn[nH]3)CC2)cc1] |
| | | N-((1R,2S,5R)-8-(1H-tetrazol-5- | | | N-((1S,2R,4S,5R)-4-methyl-8-(1H- |
| | | yl)-8-azabicyclo[3.2.1]octan-2-yl)- | | | tetrazol-5-yl)-8-azabicyclo[3.2.1]octan- |
| | | 1-(2-hydroxy-4- | | | 2-yl)-1-(p-tolyl)cyclopropane-1- |
| | | (trifluoromethyl)phenyl) | | | carboxamide |
| | | cyclopropane-1-carboxamide | | | |
| 34C* | 34 | 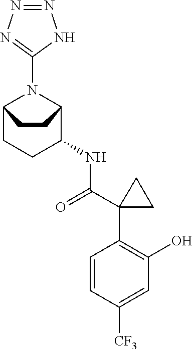 | 87D* | TA | 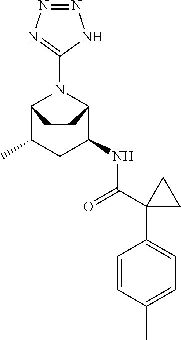 [SMILES: Cc1ccc(C2(C(=O)N[C@H]3C[C@H](C)[C@H]4CC[C@@H]3N4c3nnn[nH]3)CC2)cc1] |
| | | N-((1S,2R,5S)-8-(1H-tetrazol-5- | | | N-((1S,2S,4S,5R)-4-methyl-8-(1H- |
| | | yl)-8-azabicyclo[3.2.1]octan-2-yl)- | | | tetrazol-5-yl)-8-azabicyclo[3.2.1]octan- |
| | | 1-(2-hydroxy-4- | | | 2-yl)-1-(p-tolyl)cyclopropane-1- |
| | | (trifluoromethyl)phenyl) | | | carboxamide |
| | | cyclopropane-1-carboxamide | | | |
| 34D* | 34 | 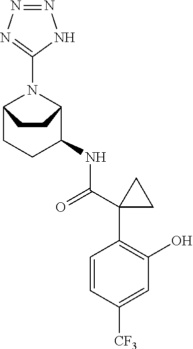 | Rac-88 | TA | 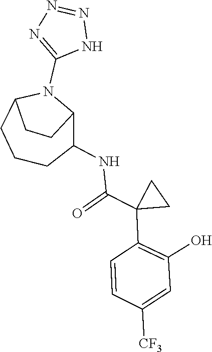 [SMILES: O=C(NC1CCCC2CCC1N2c1nnn[nH]1)C1(c2ccc(C(F)(F)F)cc2O)CC1] |
| | | N-((1S,2S,5S)-8-(1H-tetrazol-5- | | | N-(9-(1H-tetrazol-5-yl)-9- |
| | | yl)-8-azabicyclo[3.2.1]octan-2-yl)- | | | azabicyclo[4.2.1]nonan-2-yl)-1-(2- |
| | | 1-(2-hydroxy-4- | | | hydroxy-4- |
| | | (trifluoromethyl)phenyl) | | | (trifluoromethyl)phenyl)cyclopropane- |
| | | cyclopropane-1-carboxamide | | | 1-carboxamide |
| 34A- OMe * | 34 | 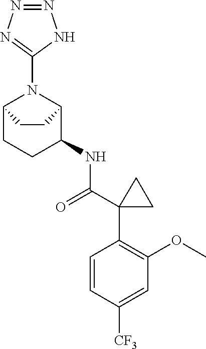 [SMILES: COc1cc(C(F)(F)F)ccc1C1(C(=O)N[C@H]2CC[C@@H]3CC[C@H]2N3c2nnn[nH]2)CC1] | 88A* | TA | 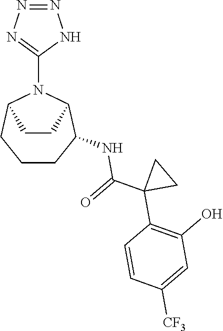 [SMILES: O=C(N[C@@H]1CCC[C@@H]2CC[C@H]1N2c1nnn[nH]1)C1(c2ccc(C(F)(F)F)cc2O)CC1] |
| | | N-((1R,2R,5R)-8-(1H-tetrazol-5- | | | N-((1R,2R,6R)-9-(1H-tetrazol-5-yl)-9- |
| | | yl)-8-azabicyclo[3.2.1]octan-2-yl)- | | | azabicyclo[4.2.1]nonan-2-yl)-1-(2- |
| | | 1-(2-methoxy-4- | | | hydroxy-4- |
| | | (trifluoromethyl)phenyl) | | | (trifluoromethyl)phenyl)cyclopropane-1- |
| | | cyclopropane-1-carboxamide | | | carboxamide |
| 34B- OMe * | 34 | 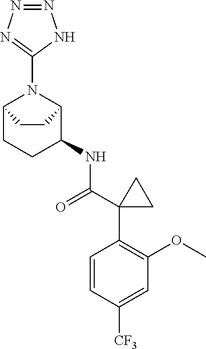 [SMILES: COc1cc(C(F)(F)F)ccc1C1(C(=O)N[C@H]2CC[C@@H]3CC[C@H]2N3c2nnn[nH]2)CC1] | 88B* | TA | 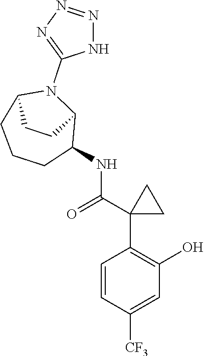 [SMILES: O=C(N[C@H]1CCC[C@@H]2CC[C@H]1N2c1nnn[nH]1)C1(c2ccc(C(F)(F)F)cc2O)CC1] |
| | | N-((1R,2S,5R)-8-(1H-tetrazol-5- | | | N-((1R,2S,6R)-9-(1H-tetrazol-5-yl)-9- |
| | | yl)-8-azabicyclo[3.2.1]octan-2-yl)- | | | azabicyclo[4.2.1]nonan-2-yl)-1-(2- |
| | | 1-(2-methoxy-4- | | | hydroxy-4- |
| | | (trifluoromethyl)phenyl) | | | (trifluoromethyl)phenyl)cyclopropane- |
| | | cyclopropane-1-carboxamide | | | 1-carboxamide |
| 34C- OMe * | 34 | 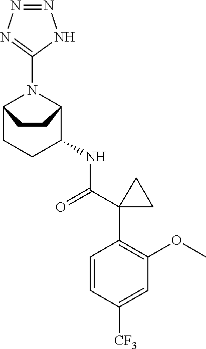 | 88C* | TA | 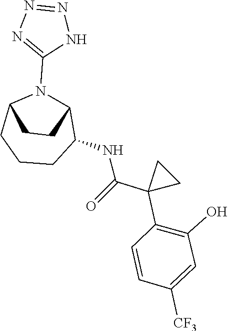 [SMILES: O=C(N[C@@H]1CCC[C@H]2CC[C@@H]1N2c1nnn[nH]1)C1(c2ccc(C(F)(F)F)cc2O)CC1] |
| | | N-((1S,2R,5S)-8-(1H-tetrazol-5- | | | N-((1S,2R,6S)-9-(1H-tetrazol-5-yl)-9- |
| | | yl)-8-azabicyclo[3.2.1]octan-2-yl)- | | | azabicyclo[4.2.1]nonan-2-yl)-1-(2- |
| | | 1-(2-methoxy-4- | | | hydroxy-4- |
| | | (trifluoromethyl)phenyl) | | | (trifluoromethyl)phenyl)cyclopropane- |
| | | cyclopropane-1-carboxamide | | | 1-carboxamide |
| 34D- OMe * | 34 | 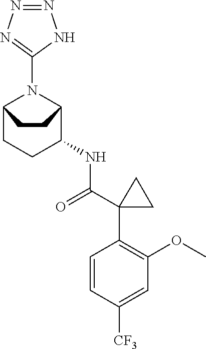 | 88D* | TA | 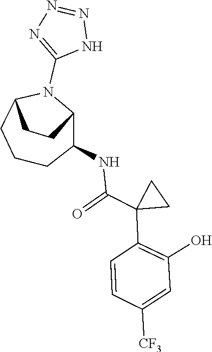 [SMILES: O=C(N[C@H]1CCC[C@H]2CC[C@@H]1N2c1nnn[nH]1)C1(c2ccc(C(F)(F)F)cc2O)CC1] |
| | | N-((1S,2S,5S)-8-(1H-tetrazol-5- | | | N-((1S,2S,6S)-9-(1H-tetrazol-5-yl)-9- |
| | | yl)-8-azabicyclo[3.2.1]octan-2-yl)- | | | azabicyclo[4.2.1]nonan-2-yl)-1-(2- |
| | | 1-(2-methoxy-4- | | | hydroxy-4- |
| | | (trifluoromethyl)phenyl) | | | (trifluoromethyl)phenyl)cyclopropane- |
| | | cyclopropane-1-carboxamide | | | 1-carboxamide |
| 35′* | 35 | 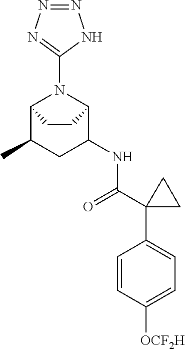 | Rac-89 | TA | 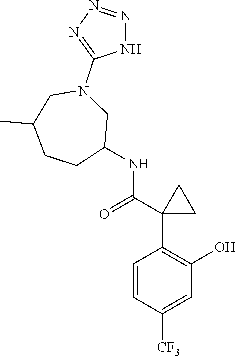 [SMILES: CC1CCC(NC(=O)C2(c3ccc(C(F)(F)F)cc3O)CC2)CN(c2nnn[nH]2)C1] |
| | | 1-(4-(difluoromethoxy)phenyl)-N- | | | 1-(2-hydroxy-4- |
| | | ((1R,4R,5S)-4-methyl-8-(1H- | | | (trifluoromethyl)phenyl)-N-(6-methyl-1- |
| | | tetrazol-5-yl)-8- | | | (1H-tetrazol-5-yl)azepan-3- |
| | | azabicyclo[3.2.1]octan-2- | | | yl)cyclopropane-1-carboxamide |
| | | yl)cyclopropane-1-carboxamide | | | |
| 35″* | 35 | 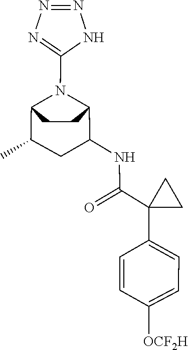 | 89A* | TA | 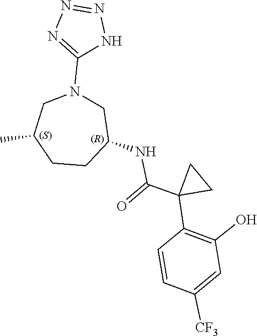 |
| | | 1-(4-(difluoromethoxy)phenyl)-N- | | | 1-(2-hydroxy-4- |
| | | ((1S,4S,5R)-4-methyl-8-(1H- | | | (trifluoromethyl)phenyl)-N-((3R,6S)-6- |
| | | tetrazol-5-yl)-8- | | | methyl-1-(1H-tetrazol-5-yl)azepan-3- |
| | | azabicyclo[3.2.1]octan-2- | | | yl)cyclopropane-1-carboxamide |
| | | yl)cyclopropane-1-carboxamide | | | |
| 35′′′ | 35 | 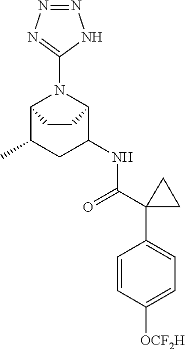 | 89B* | TA | 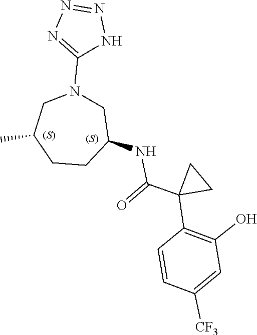 |
| | | 1-(4-(difluoromethoxy)phenyl)-N- | | | 1-(2-hydroxy-4- |
| | | ((1R,4S,5S)-4-methyl-8-(1H- | | | (trifluoromethyl)phenyl)-N-((3S,6S)-6- |
| | | tetrazol-5-yl)-8- | | | methyl-1-(1H-tetrazol-5-yl)azepan-3- |
| | | azabicyclo[3.2.1]octan-2- | | | yl)cyclopropane-1-carboxamide |
| | | yl)cyclopropane-1-carboxamide | | | |
| 35′′′′ | 35 | 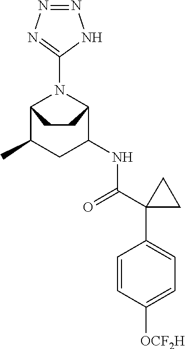 | 89C* | TA | 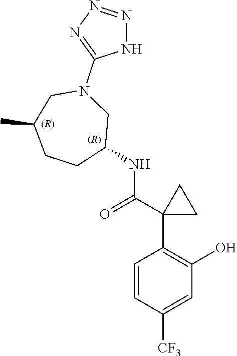 |
| | | 1-(4-(difluoromethoxy)phenyl)-N- | | | 1-(2-hydroxy-4- |
| | | ((1S,4R,5R)-4-methyl-8-(1H- | | | (trifluoromethyl)phenyl)-N-((3R,6R)-6- |
| | | tetrazol-5-yl)-8- | | | methyl-1-(1H-tetrazol-5-yl)azepan-3- |
| | | azabicyclo[3.2.1]octan-2- | | | yl)cyclopropane-1-carboxamide |
| | | yl)cyclopropane-1-carboxamide | | | |
| 35A* | 35 | 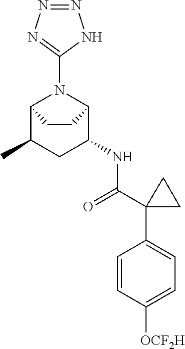 | 89D* | TA | 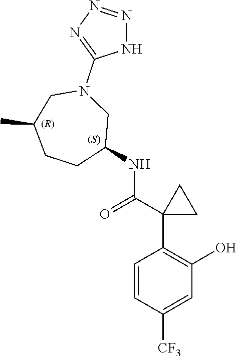 |
| | | 1-(4-(difluoromethoxy)phenyl)-N- | | | 1-(2-hydroxy-4- |
| | | ((1R,2R,4R,5S)-4-methyl-8-(1H- | | | (trifluoromethyl)phenyl)-N-((3S,6R)-6- |
| | | tetrazol-5-yl)-8- | | | methyl-1-(1H-tetrazol-5-yl)azepan-3- |
| | | azabicyclo [3.2.1]octan-2- | | | yl)cyclopropane-1-carboxamide |
| | | yl)cyclopropane-1-carboxamide | | | |
| 35B* | 35 | 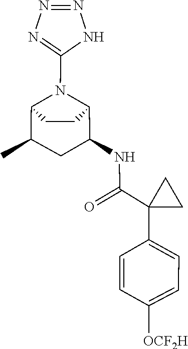 | Rac-90 | TA | 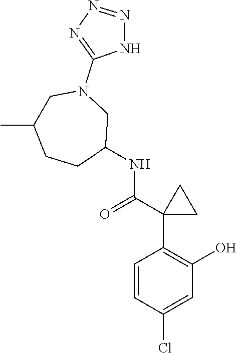 [SMILES: CC1CCC(NC(=O)C2(c3ccc(Cl)cc3O)CC2)CN(c2nnn[nH]2)C1] |
| | | 1-(4-(difluoromethoxy)phenyl)-N- | | | 1-(4-chloro-2-hydroxyphenyl)-N-(6- |
| | | ((1R,2S,4R,5S)-4-methyl-8-(1H- | | | methyl-1-(1H-tetrazol-5-yl)azepan-3- |
| | | tetrazol-5-yl)-8- | | | yl)cyclopropane-1-carboxamide |
| | | azabicyclo[3.2.1]octan-2- | | | |
| | | yl)cyclopropane-1-carboxamide | | | |
| 35C* | 35 | 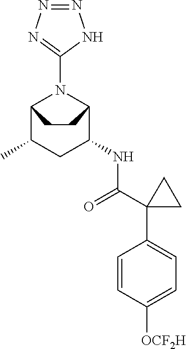 | 90A* | TA | 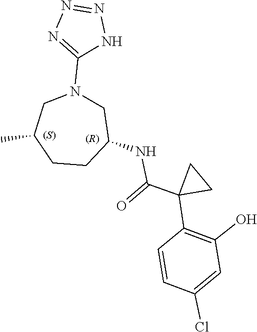 |
| | | 1-(4-(difluoromethoxy)phenyl)-N- | | | 1-(4-chloro-2-hydroxyphenyl)-N- |
| | | ((1S,2R,4S,5R)-4-methyl-8-(1H- | | | ((3R,6S)-6-methyl-1-(1H-tetrazol-5- |
| | | tetrazol-5-yl)-8- | | | yl)azepan-3-yl)cyclopropane-1- |
| | | azabicyclo[3.2.1]octan-2- | | | carboxamide |
| | | yl)cyclopropane-1-carboxamide | | | |
| 35D* | 35 | 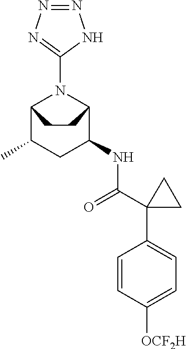 | 90B* | TA | 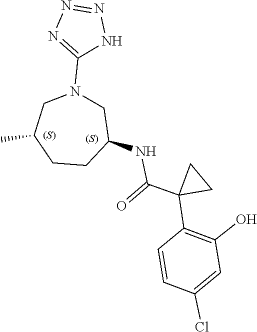 |
| | | 1-(4-(difluoromethoxy)phenyl)-N- | | | 1-(4-chloro-2-hydroxyphenyl)-N- |
| | | ((1S,2S,4S,5R)-4-methyl-8-(1H- | | | ((3S,6S)-6-methyl-1-(1H-tetrazol-5- |
| | | tetrazol-5-yl)-8- | | | yl)azepan-3-yl)cyclopropane-1- |
| | | azabicyclo[3.2.1]octan-2- | | | carboxamide |
| | | yl)cyclopropane-1-carboxamide | | | |
| 35E* | 35 | 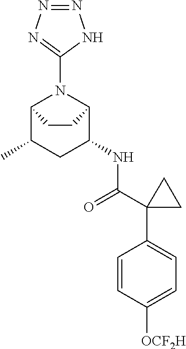 | 90C* | TA | 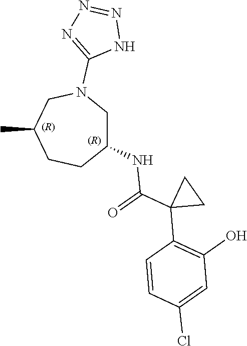 |
| | | 1-(4-(difluoromethoxy)phenyl)-N- | | | 1-(4-chloro-2-hydroxyphenyl)-N- |
| | | ((1R,2R,4S,5S)-4-methyl-8-(1H- | | | ((3R,6R)-6-methyl-1-(1H-tetrazol-5- |
| | | tetrazol-5-yl)-8- | | | yl)azepan-3-yl)cyclopropane-1- |
| | | azabicyclo[3.2.1]octan-2- | | | carboxamide |
| | | yl)cyclopropane-1-carboxamide | | | |
| 35F* | 35 | 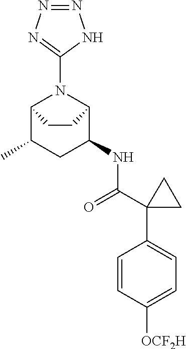 | 90D* | TA | 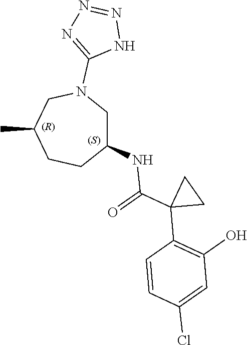 |
| | | 1-(4-(difluoromethoxy)phenyl)-N- | | | 1-(4-chloro-2-hydroxyphenyl)-N- |
| | | ((1R,2S,4S,5S)-4-methyl-8-(1H- | | | ((3S,6R)-6-methyl-1-(1H-tetrazol-5- |
| | | tetrazol-5-yl)-8- | | | yl)azepan-3-yl)cyclopropane-1- |
| | | azabicyclo[3.2.1]octan-2- | | | carboxamide |
| | | yl)cyclopropane-1-carboxamide | | | |
| 35G* | 35 | 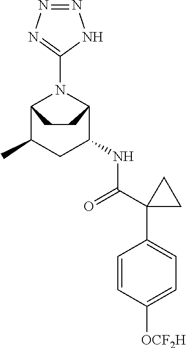 | Rac-91 | TA | 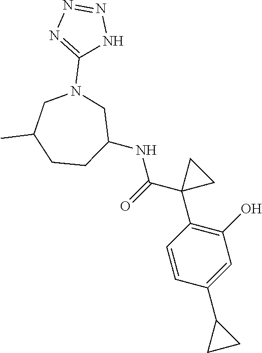 [SMILES: CC1CCC(NC(=O)C2(c3ccc(C4CC4)cc3O)CC2)CN(c2nnn[nH]2)C1] |
| | | 1-(4-(difluoromethoxy)phenyl)-N- | | | 1-(4-cyclopropyl-2-hydroxyphenyl)-N- |
| | | ((1S,2R,4R,5R)-4-methyl-8-(1H- | | | (6-methyl-1-(1H-tetrazol-5-yl)azepan-3- |
| | | tetrazol-5-yl)-8- | | | yl)cyclopropane-1-carboxamide |
| | | azabicyclo[3.2.1]octan-2- | | | |
| | | yl)cyclopropane-1-carboxamide | | | |
| 35H* | 35 | 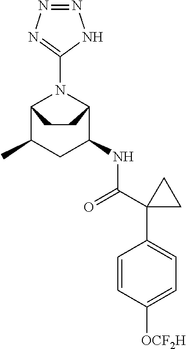 | 91A* | TA | 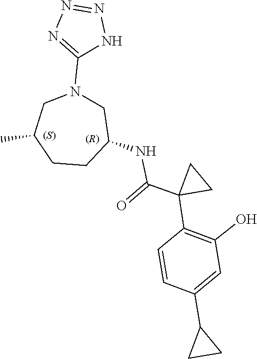 |
| | | 1-(4-(difluoromethoxy)phenyl)-N- | | | 1-(4-cyclopropyl-2-hydroxyphenyl)-N- |
| | | ((1S,2S,4R,5R)-4-methyl-8-(1H- | | | ((3R,6S)-6-methyl-1-(1H-tetrazol-5- |
| | | tetrazol-5-yl)-8- | | | yl)azepan-3-yl)cyclopropane-1- |
| | | azabicyclo[3.2.1]octan-2- | | | carboxamide |
| | | yl)cyclopropane-1-carboxamide | | | |
| 36A* | 36 | 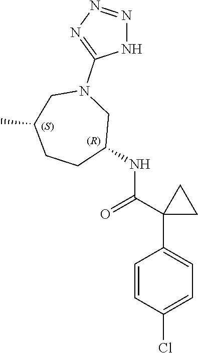 | 91B* | TA | 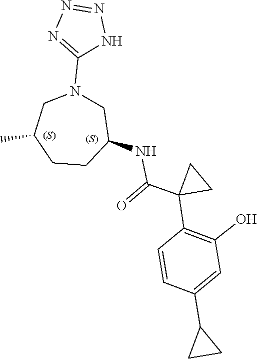 |
| | | 1-(4-chlorophenyl)-N-((3R,6S)-6- | | | 1-(4-cyclopropyl-2-hydroxyphenyl)-N- |
| | | methyl-1-(1H-tetrazol-5-yl)azepan- | | | ((3S,6S)-6-methyl-1-(1H-tetrazol-5- |
| | | 3-yl)cyclopropane-1-carboxamide | | | yl)azepan-3-yl)cyclopropane-1- |
| | | | | | carboxamide |
| 36B* | 36 | 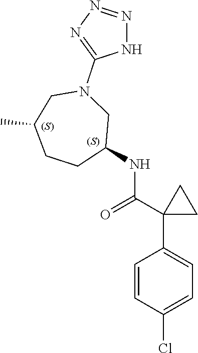 | 91C* | TA | 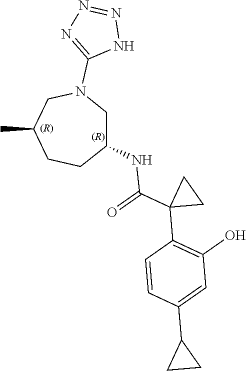 |
| | | 1-(4-chlorophenyl)-N-((3S,6S)-6- | | | 1-(4-cyclopropyl-2-hydroxyphenyl)-N- |
| | | methyl-1-(1H-tetrazol-5-yl)azepan- | | | ((3R,6R)-6-methyl-1-(1H-tetrazol-5- |
| | | 3-yl)cyclopropane-1-carboxamide | | | yl)azepan-3-yl)cyclopropane-1- |
| | | | | | carboxamide |
| 36C* | 36 | 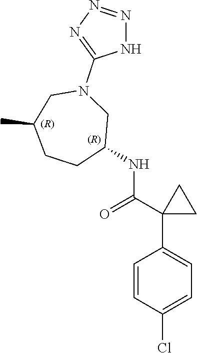 | 91D* | TA | 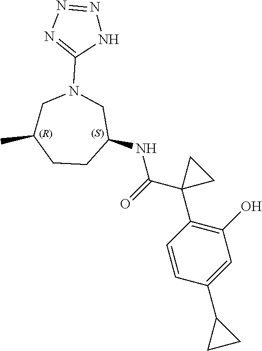 |
| | | 1-(4-chlorophenyl)-N-((3R,6R)-6- | | | 1-(4-cyclopropyl-2-hydroxyphenyl)-N- |
| | | methyl-1-(1H-tetrazol-5-yl)azepan- | | | ((3S,6R)-6-methyl-1-(1H-tetrazol-5- |
| | | 3-yl)cyclopropane-1-carboxamide | | | yl)azepan-3-yl)cyclopropane-1- |
| | | | | | carboxamide |
| 36D* | 36 | 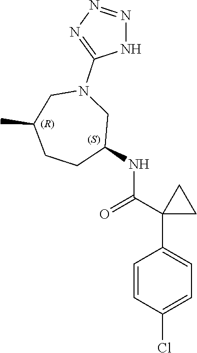 | Rac-92 | TA | 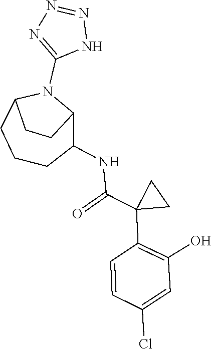 [SMILES: O=C(NC1CCCC2CCC1N2c1nnn[nH]1)C1(c2ccc(Cl)cc2O)CC1] |
| | | 1-(4-chlorophenyl)-N-((3S,6R)-6- | | | N-(9-(1H-tetrazol-5-yl)-9- |
| | | methyl-1-(1H-tetrazol-5-yl)azepan- | | | azabicyclo[4.2.1]nonan-2-yl)-1-(4- |
| | | 3-yl)cyclopropane-1-carboxamide | | | chloro-2-hydroxyphenyl)cyclopropane- |
| | | | | | 1-carboxamide |
| Rac- 37 | TA | 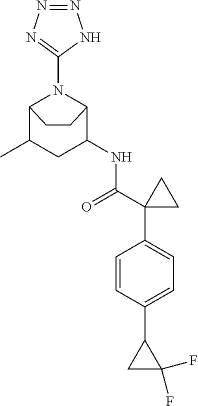 [SMILES: CC1CC(NC(=O)C2(c3ccc(C4CC4(F)F)cc3)CC2)C2CCC1N2c1nnn[nH]1] | 92A* | TA | 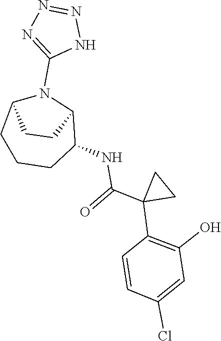 [SMILES: O=C(N[C@@H]1CCC[C@@H]2CC[C@H]1N2c1nnn[nH]1)C1(c2ccc(Cl)cc2O)CC1] N-((1R,2R,6R)-9-(1H-tetrazol-5-yl)-9- azabicyclo[4.2.1]nonan-2-yl)-1-(4- chloro-2-hydroxyphenyl)cyclopropane- 1-carboxamide |
| | | 4-methyl-8-(1H-tetrazol-5-yl)-8- | | | |
| | | azabicyclo[3.2.1]octan-2-yl)-1-(4- | | | |
| | | (2,2- | | | |
| | | difluorocyclopropyl)phenyl) | | | |
| | | cyclopropane-1-carboxamide | | | |
| 37′* | TA | 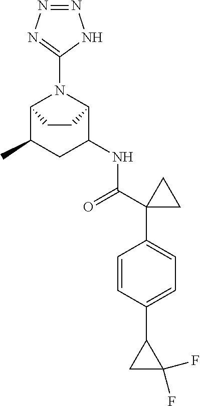 [SMILES: C[C@@H]1CC(NC(=O)C2(c3ccc(C4CC4(F)F)cc3)CC2)[C@H]2CC[C@@H]1N2c1nnn[nH]1] | 92B* | TA | 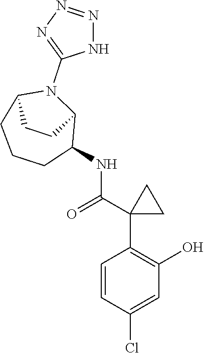 [SMILES: O=C(N[C@H]1CCC[C@@H]2CC[C@H]1N2c1nnn[nH]1)C1(c2ccc(Cl)cc2O)CC1] N-((1R,2S,6R)-9-(1H-tetrazol-5-yl)-9- azabicyclo[4.2.1]nonan-2-yl)-1-(4- chloro-2-hydroxyphenyl)cyclopropane- |
| | | | | | 1-carboxamide |
| | | N-((1R,2R,5S)-4-methyl-8-(1H- | | | |
| | | tetrazol-5-yl)-8- | | | |
| | | azabicyclo[3.2.1]octan-2-yl)-1-(4- | | | |
| | | (2,2- | | | |
| | | difluorocyclopropyl)phenyl) | | | |
| | | cyclopropane-1-carboxamide | | | |
| 37″* | TA | 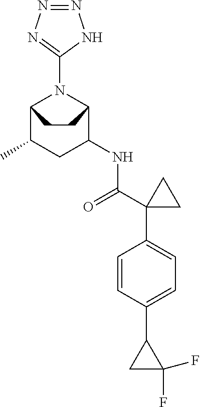 [SMILES: C[C@H]1CC(NC(=O)C2(c3ccc(C4CC4(F)F)cc3)CC2)[C@@H]2CC[C@H]1N2c1nnn[nH]1] | 92C* | TA | 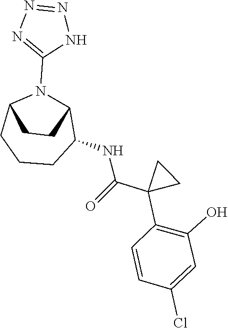 [SMILES: O=C(N[C@@H]1CCC[C@H]2CC[C@@H]1N2c1nnn[nH]1)C1(c2ccc(Cl)cc2O)CC1] N-((1S,2R,6S)-9-(1H-tetrazol-5-yl)-9- azabicyclo[4.2.1]nonan-2-yl)-1-(4- chloro-2-hydroxyphenyl)cyclopropane- 1-carboxamide |
| | | N-((1S,2S,5R)-4-methyl-8-(1H- | | | |
| | | tetrazol-5-yl)-8- | | | |
| | | azabicyclo[3.2.1]octan-2-yl)-1-(4- | | | |
| | | (2,2- | | | |
| | | difluorocyclopropyl)phenyl) | | | |
| | | cyclopropane-1-carboxamide | | | |
| 37A* | TA | 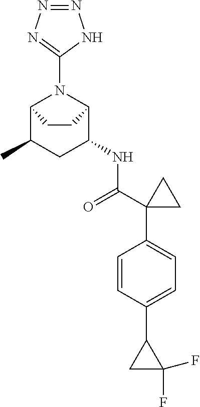 [SMILES: C[C@@H]1C[C@@H](NC(=O)C2(c3ccc(C4CC4(F)F)cc3)CC2)[C@H]2CC[C@@H]1N2c1nnn[nH]1] | 92D* | TA | 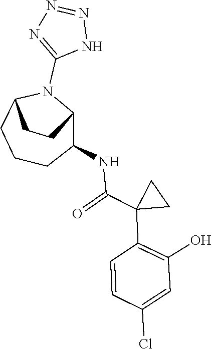 [SMILES: O=C(N[C@H]1CCC[C@H]2CC[C@@H]1N2c1nnn[nH]1)C1(c2ccc(Cl)cc2O)CC1] N-((1S,2S,6S)-9-(1H-tetrazol-5-yl)-9- azabicyclo[4.2.1]nonan-2-yl)-1-(4- chloro-2-hydroxyphenyl)cyclopropane- |
| | | | | | 1-carboxamide |
| | | N-((1R,2R,4R,5S)-4-methyl-8- | | | |
| | | (1H-tetrazol-5-yl)-8- | | | |
| | | azabicyclo[3.2.1]octan-2-yl)-1-(4- | | | |
| | | 2,2- | | | |
| | | difluorocyclopropyl)phenyl) | | | |
| | | cyclopropane-1-carboxamide | | | |
| 37B* | TA | 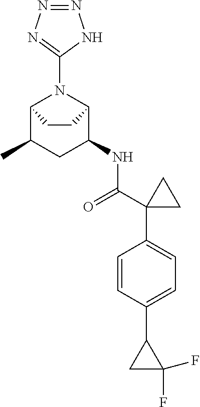 [SMILES: C[C@@H]1C[C@H](NC(=O)C2(c3ccc(C4CC4(F)F)cc3)CC2)[C@H]2CC[C@@H]1N2c1nnn[nH]1] | Rac-93 | TA | 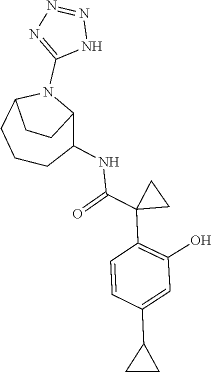 [SMILES: O=C(NC1CCCC2CCC1N2c1nnn[nH]1)C1(c2ccc(C3CC3)cc2O)CC1] N-(9-(1H-tetrazol-5-yl)-9- azabicyclo[4.2.1]nonan-2-yl)-1-(4- |
| | | | | | cyclopropyl-2- |
| | | N-((1R,2S,4R,5S)-4-methyl-8-(1H- | | | hydroxyphenyl)cyclopropane-1- |
| | | tetrazol-5-yl)-8- | | | carboxamide |
| | | azabicyclo[3.2.1]octan-2-yl)-1-(4- | | | |
| | | (2,2- | | | |
| | | difluorocyclopropyl)phenyl) | | | |
| | | cyclopropane-1-carboxamide | | | |
| 37C* | TA | 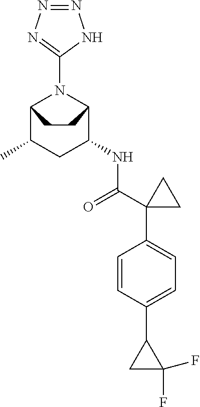 [SMILES: C[C@H]1C[C@@H](NC(=O)C2(c3ccc(C4CC4(F)F)cc3)CC2)[C@@H]2CC[C@H]1N2c1nnn[nH]1] | 93A* | TA | 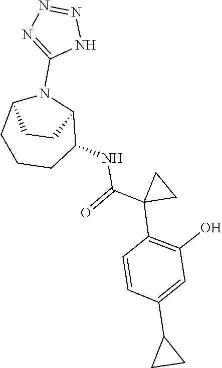 [SMILES: O=C(N[C@@H]1CCC[C@@H]2CC[C@H]1N2c1nnn[nH]1)C1(c2ccc(C3CC3)cc2O)CC1] N-((1R,2R,6R)-9-(1H-tetrazol-5-yl)-9- azabicyclo[4.2.1]nonan-2-yl)-1-(4- |
| | | | | | cyclopropyl-2- |
| | | N-((1S,2R,4S,5R)-4-methyl-8-(1H- | | | hydroxyphenyl)cyclopropane-1- |
| | | tetrazol-5-yl)-8- | | | carboxamide |
| | | azabicyclo[3.2.1]octan-2-yl)-1-(4- | | | |
| | | (2,2- | | | |
| | | difluorocyclopropyl)phenyl) | | | |
| | | cyclopropane-1-carboxamide | | | |
| 37D* | TA | 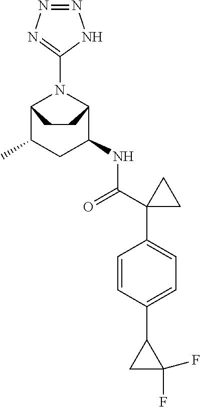 [SMILES: C[C@H]1C[C@H](NC(=O)C2(c3ccc(C4CC4(F)F)cc3)CC2)[C@@H]2CC[C@H]1N2c1nnn[nH]1] | 93B* | TA | 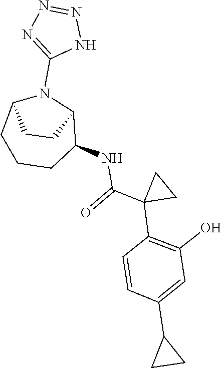 [SMILES: O=C(N[C@H]1CCC[C@@H]2CC[C@H]1N2c1nnn[nH]1)C1(c2ccc(C3CC3)cc2O)CC1] N-((1R,2S,6R)-9-(1H-tetrazol-5-yl)-9- azabicyclo[4.2.1]nonan-2-yl)-1-(4- |
| | | | | | cyclopropyl-2- |
| | | N-((1S,2S,4S,5R)-4-methyl-8-(1H- | | | hydroxyphenyl)cyclopropane-1- |
| | | tetrazol-5-yl)-8- | | | carboxamide |
| | | azabicyclo[3.2.1]octan-2-yl)-1-(4- | | | |
| | | (2,2- | | | |
| | | difluorocyclopropyl)phenyl) | | | |
| | | cyclopropane-1-carboxamide | | | |
| 38A | TA | 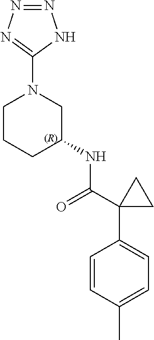 (R)-N-(1-(1H-tetrazol-5- | 93C* | TA | 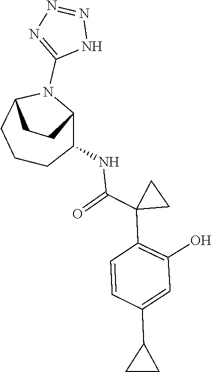 [SMILES: O=C(N[C@@H]1CCC[C@H]2CC[C@@H]1N2c1nnn[nH]1)C1(c2ccc(C3CC3)cc2O)CC1] |
| | | yl)piperidin-3-yl)-1-(p- | | | |
| | | tolyl)cyclopropane-1-carboxamide | | | N-((1S,2R,6S)-9-(1H-tetrazol-5-yl)-9- |
| | | | | | azabicyclo[4.2.1]nonan-2-yl)-1-(4- |
| | | | | | cyclopropyl-2- |
| | | | | | hydroxyphenyl)cyclopropane-1- |
| | | | | | carboxamide |
| 38B | TA | 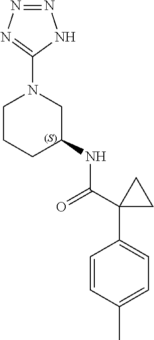 (S)-N-(1-(1H-tetrazol-5- | 93D* | TA | 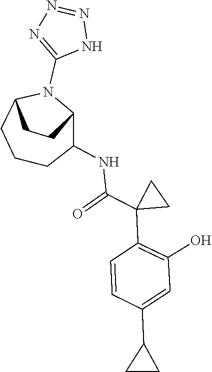 [SMILES: O=C(NC1CCC[C@H]2CC[C@@H]1N2c1nnn[nH]1)C1(c2ccc(C3CC3)cc2O)CC1] |
| | | yl)piperidin-3-yl)-1-(p- | | | |
| | | tolyl)cyclopropane-1-carboxamide | | | N-((1S,2S,6S)-9-(1H-tetrazol-5-yl)-9- |
| | | | | | azabicyclo[4.2.1]nonan-2-yl)-1-(4- |
| | | | | | cyclopropyl-2- |
| | | | | | hydroxyphenyl)cyclopropane-1- |
| | | | | | carboxamide |
| 39A | TA | 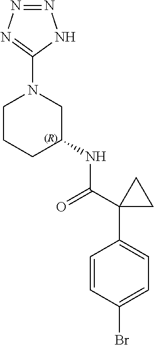 1-(4-bromophenyl)-N-[(3R)-1- (1H-tetrazol-5-yl)-3- piperidyl]cyclopropanecarboxamide | 94A* | TA | 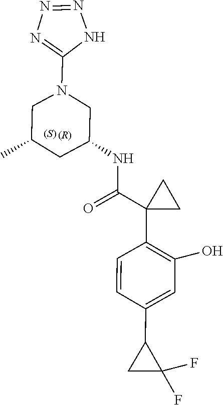 |
| | | | | | 1-(4-(2,2-difluorocyclopropyl)-2- |
| | | | | | hydroxyphenyl)-N-((3R,5S)-5-methyl-1- |
| | | | | | (1H-tetrazol-5-yl)piperidin-3- |
| | | | | | yl)cyclopropane-1-carboxamide |
| 39B | TA | 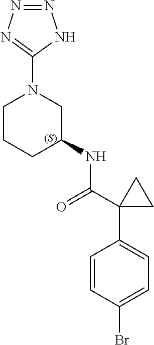 1-(4-bromophenyl)-N-[(3S)-1-(1H- tetrazol-5-yl)-3- piperidyl]cyclopropanecarboxamide | 94B* | TA | 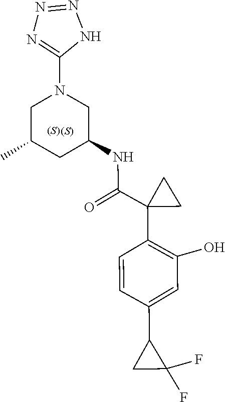 |
| | | | | | 1-(4-(2,2-difluorocyclopropyl)-2- |
| | | | | | hydroxyphenyl)-N-((3S,5S)-5-methyl-1- |
| | | | | | (1H-tetrazo1-5-yl)piperidin-3- |
| | | | | | yl)cyclopropane-1-carboxamide |
| 40A | TA | 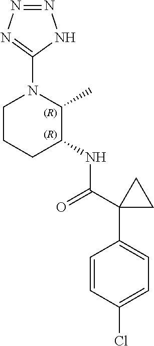 1-(4-chlorophenyl)-N-[(2R,3R)-2- methyl-1-(1H-tetrazol-5-yl)-3- piperidyl]cyclopropanecarboxamide | 94C* | TA | 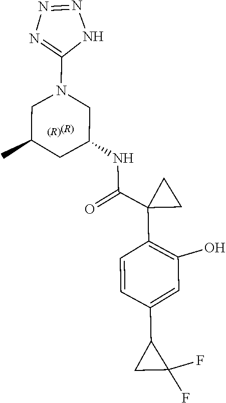 |
| | | | | | 1-(4-(2,2-difluorocyclopropyl)-2- |
| | | | | | hydroxyphenyl)-N-((3R,5R)-5-methyl-1- |
| | | | | | (1H-tetrazol-5-yl)piperidin-3- |
| 40B | TA | 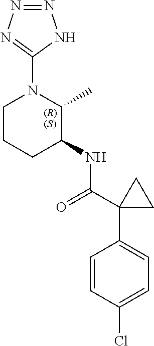 | 94D* | TA | 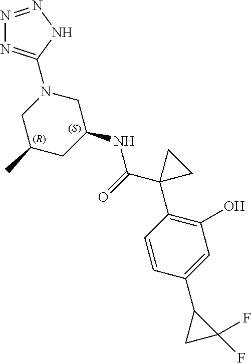 |
| | | 1-(4-chlorophenyl)-N-[(2R,3S)-2- | | | 1-(4-(2,2-difluorocyclopropyl)-2- |
| | | methyl-1-(1H-tetrazol-5-yl)-3- | | | hydroxyphenyl)-N-((3S,5R)-5-methyl-1- |
| | | piperidyl]cyclopropanecarboxamide | | | (1H-tetrazol-5-yl)piperidin-3- |
| | | | | | yl)cyclopropane-1-carboxamide |
| 40C | TA | 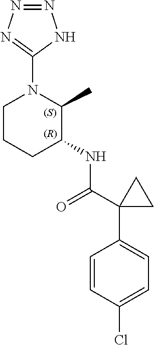 | 95A* | TA | 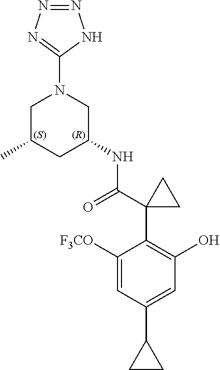 |
| | | 1-(4-chlorophenyl)-N-[(2S,3R)-2- | | | 1-(4-cyclopropyl-2-hydroxy-6- |
| | | methyl-1-(1H-tetrazol-5-yl)-3- | | | (trifluoromethoxy)phenyl)-N-((3R,5S)- |
| | | piperidyl]cyclopropanecarboxamide | | | 5-methyl-1-(1H-tetrazol-5-yl)piperidin- |
| | | | | | 3-yl)cyclopropane-1-carboxamide |
| 40D | TA | 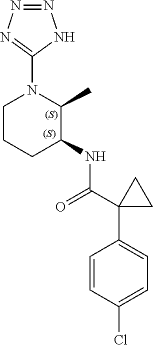 | 95B* | TA | 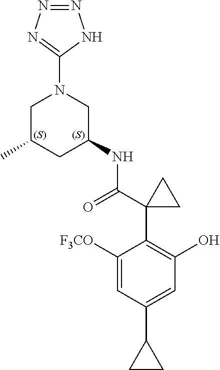 |
| | | 1-(4-chlorophenyl)-N-[(2S,3S)-2- | | | 1-(4-cyclopropyl-2-hydroxy-6- |
| | | methyl-1-(1H-tetrazol-5-yl)-3- | | | (trifluoromethoxy)phenyl)-N-((3S,5S)-5- |
| | | piperidyl]cyclopropanecarboxamide | | | methyl-1-(1H-tetrazol-5-yl)piperidin-3- |
| | | | | | yl)cyclopropane-1-carboxamide |
| 41A | TA | 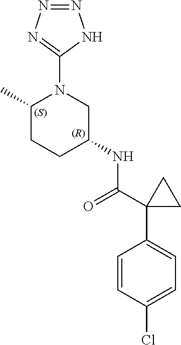 | 95C* | TA | 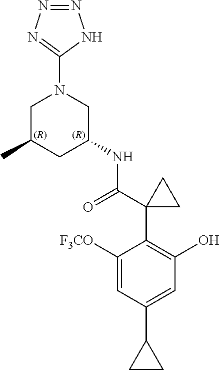 |
| | | 1-(4-chlorophenyl)-N-[(3R,6S)-6- | | | 1-(4-cyclopropyl-2-hydroxy-6- |
| | | methyl-1-(1H-tetrazol-5-yl)-3- | | | (trifluoromethoxy)phenyl)-N-((3R,5R)- |
| | | piperidyl]cyclopropanecarboxamide | | | 5-methyl-1-(1H-tetrazol-5-yl)piperidin- |
| | | | | | 3-yl)cyclopropane-1-carboxamide |
| 41B | TA | 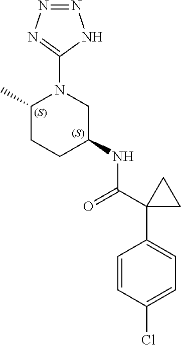 | 95D* | TA | 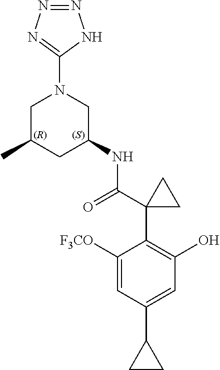 |
| | | 1-(4-chlorophenyl)-N-[(3S,6S)-6- | | | 1-(4-cyclopropyl-2-hydroxy-6- |
| | | methyl-1-(1H-tetrazol-5-yl)-3- | | | (trifluoromethoxy)phenyl)-N-((3S,5R)- |
| | | piperidyl]cyclopropanecarboxamide | | | 5-methyl-1-(1H-tetrazol-5-yl)piperidin- |
| | | | | | 3-yl)cyclopropane-1-carboxamide |
| 41C | TA | 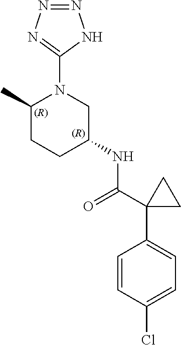 | 96A* | TA | 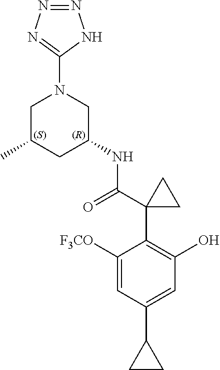 |
| | | 1-(4-chlorophenyl)-N-[(3R,6R)-6- | | | 1-(4-cyclopropyl-2-hydroxy-6- |
| | | methyl-1-(1H-tetrazol-5-yl)-3- | | | (trifluoromethyl)phenyl)-N-((3R,5S)-5- |
| | | piperidyl]cyclopropanecarboxamide | | | methyl-1-(1H-tetrazol-5-yl)piperidin-3- |
| | | | | | yl)cyclopropane-1-carboxamide |
| 41D | TA | 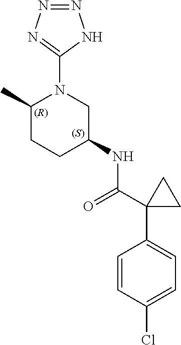 | 96B* | TA | 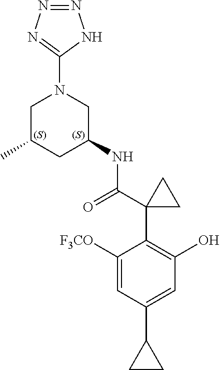 |
| | | 1-(4-chlorophenyl)-N-[(3S,6R)-6- | | | 1-(4-cyclopropyl-2-hydroxy-6- |
| | | methyl-1-(1H-tetrazol-5-yl)-3- | | | (trifluoromethyl)phenyl)-N-((3S,5S)-5- |
| | | piperidyl]cyclopropanecarboxamide | | | methyl-1-(1H-tetrazo1-5-yl)piperidin-3- |
| | | | | | yl)cyclopropane-1-carboxamide |
| Rac- 42 | | 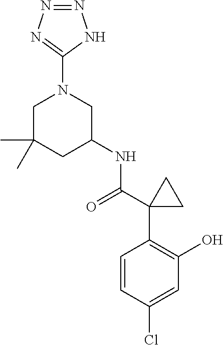 [SMILES: CC1(C)CC(NC(=O)C2(c3ccc(Cl)cc3O)CC2)CN(c2nnn[nH]2)C1] | 96C* | TA | 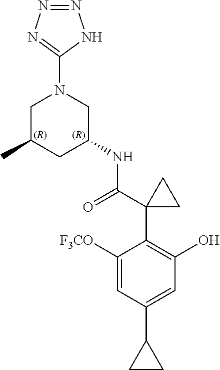 |
| | | 1-(4-chloro-2-hydroxyphenyl)-N- | | | 1-(4-cyclopropyl-2-hydroxy-6- |
| | | (5,5-dimethyl-1-(1H-tetrazol-5- | | | (trifluoromethyl)phenyl)-N-((3R,5R)-5- |
| | | yl)piperidin-3-yl)cyclopropane-1- | | | methyl-1-(1H-tetrazol-5-yl)piperidin-3- |
| | | carboxamide | | | yl)cyclopropane-1-carboxamide |
| 42A* | TA | 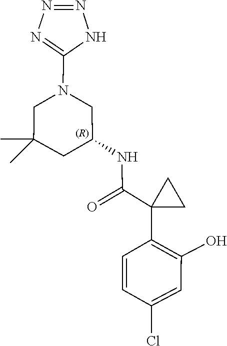 | 96D* | TA | 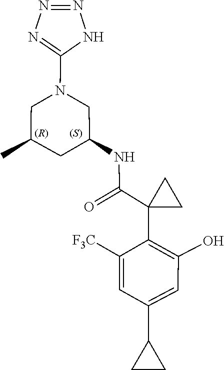 |
| | | (R)-1-(4-chloro-2-hydroxyphenyl)- | | | 1-(4-cyclopropyl-2-hydroxy-6- |
| | | N-(5,5-dimethyl-1-(1H-tetrazol-5- | | | (trifluoromethyl)phenyl)-N-((3S,5R)-5- |
| | | yl)piperidin-3-yl)cyclopropane-1- | | | methyl-1-(1H-tetrazol-5-yl)piperidin-3- |
| | | carboxamide | | | yl)cyclopropane-1-carboxamide |
| 42B* | TA | 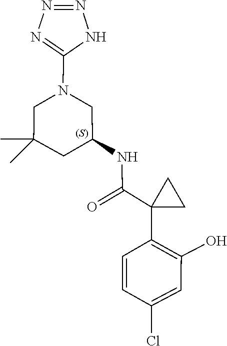 | 97A* | TA | 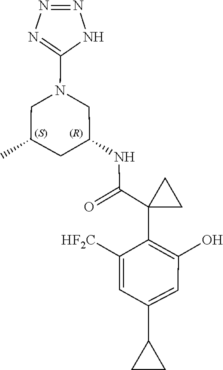 |
| | | (S)-1-(4-chloro-2-hydroxyphenyl)- | | | 1-(4-cyclopropyl-2-(difluoromethyl)-6- |
| | | N-(5,5-dimethyl-1-(1H-tetrazol-5- | | | hydroxyphenyl)-N-((3R,5S)-5-methyl-1- |
| | | yl)piperidin-3-yl)cyclopropane-1- | | | (1H-tetrazol-5-yl)piperidin-3- |
| | | carboxamide | | | yl)cyclopropane-1-carboxamide |
| Rac- 43 | TA | 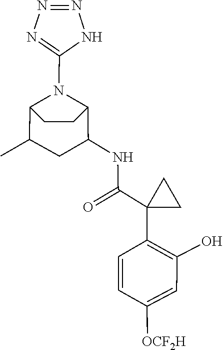 | 97B* | TA | 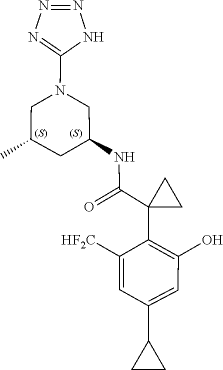 |
| | | 4-methyl-8-(1H-tetrazol-5-yl)-8- | | | 1-(4-cyclopropyl-2-(difluoromethyl)-6- |
| | | azabicyclo[3.2.1]octan-2-yl)-1-(4- | | | hydroxyphenyl)-N-((3S,5S)-5-methyl-1- |
| | | (difluoromethoxy)-2- | | | (1H-tetrazol-5-yl)piperidin-3- |
| | | hydroxyphenyl)cyclopropane-1- | | | yl)cyclopropane-1-carboxamide |
| | | carboxamide | | | |
| 43′* | TA | 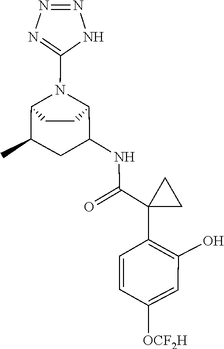 | 97C* | TA | 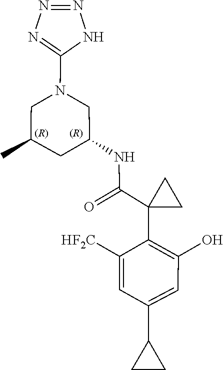 |
| | | N-((1R,2R,5S)-4-methyl-8-(1H- | | | 1-(4-cyclopropyl-2-(difluoromethyl)-6- |
| | | tetrazol-5-yl)-8- | | | hydroxyphenyl)-N-((3R,5R)-5-methyl-1- |
| | | azabicyclo[3.2.1]octan-2-yl)-1-(4- | | | (1H-tetrazol-5-yl)piperidin-3- |
| | | (difluoromethoxy)-2- | | | yl)cyclopropane-1-carboxamide |
| | | hydroxyphenyl)cyclopropane-1- | | | |
| | | carboxamide | | | |
| 43″* | TA | 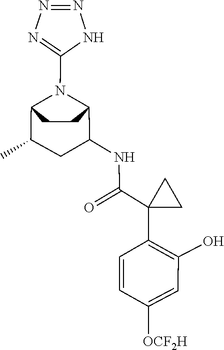 | 97D* | TA | 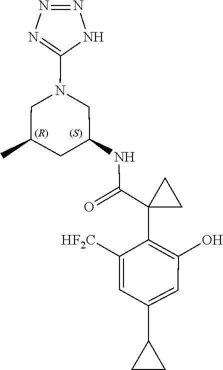 |
| | | N-((1S,2S,5R)-4-methyl-8-(1H- | | | 1-(4-cyclopropyl-2-(difluoromethyl)-6- |
| | | tetrazol-5-yl)-8- | | | hydroxyphenyl)-N-((3S,5R)-5-methyl-1- |
| | | azabicyclo[3.2.1]octan-2-yl)-1-(4- | | | (1H-tetrazo1-5-yl)piperidin-3- |
| | | (difluoromethoxy)-2- | | | yl)cyclopropane-1-carboxamide |
| | | hydroxyphenyl)cyclopropane-1- | | | |
| | | carboxamide | | | |
| 43A* | TA | 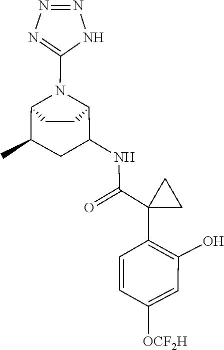 | Rac-98 | TA | 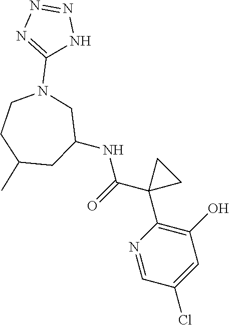 [SMILES: CC1CCN(c2nnn[nH]2)CC(NC(=O)C2(c3ncc(Cl)cc3O)CC2)C1] |
| | | N-((1R,2R,4R,5S)-4-methyl-8- | | | 1-(5-chloro-3-hydroxypyridin-2-yl)-N- |
| | | (1H-tetrazol-5-yl)-8- | | | (5-methyl-1-(1H-tetrazol-5-yl)azepan-3- |
| | | azabicyclo[3.2.1]octan-2-yl)-1-(4- | | | yl)cyclopropane-1-carboxamide |
| | | (difluoromethoxy)-2- | | | |
| | | hydroxyphenyl)cyclopropane-1- | | | |
| | | carboxamide | | | |
| 43B* | TA | 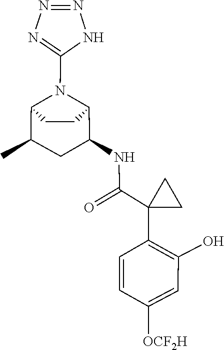 | 98A* | TA | 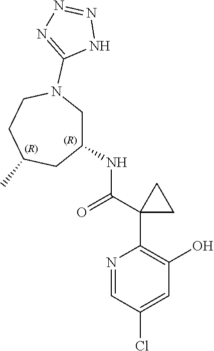 |
| | | N-((1R,2S,4R,5S)-4-methyl-8-(1H- | | | 1-(5-chloro-3-hydroxypyridin-2-yl)-N- |
| | | tetrazol-5-yl)-8- | | | ((3R,5R)-5-methyl-1-(1H-tetrazol-5- |
| | | azabicyclo[3.2.1]octan-2-yl)-1-(4- | | | yl)azepan-3-yl)cyclopropane-1- |
| | | (difluoromethoxy)-2- | | | carboxamide |
| | | hydroxyphenyl)cyclopropane-1- | | | |
| | | carboxamide | | | |
| 43C* | TA | 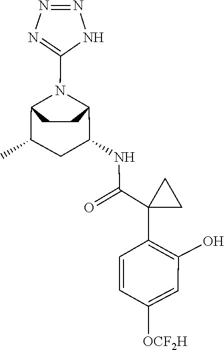 | 98B* | TA | 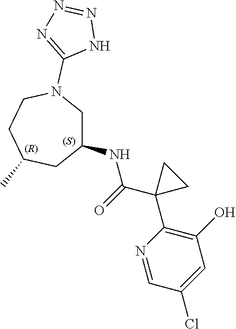 |
| | | N-((1S,2R,4S,5R)-4-methyl-8-(1H- | | | 1-(5-chloro-3-hydroxypyridin-2-yl)-N- |
| | | tetrazol-5-yl)-8- | | | ((3S,5R)-5-methyl-1-(1H-tetrazol-5- |
| | | azabicyclo[3.2.1]octan-2-yl)-1-(4- | | | yl)azepan-3-yl)cyclopropane-1- |
| | | (difluoromethoxy)-2- | | | carboxamide |
| | | hydroxyphenyl)cyclopropane-1- | | | |
| | | carboxamide | | | |
| 43D* | TA | 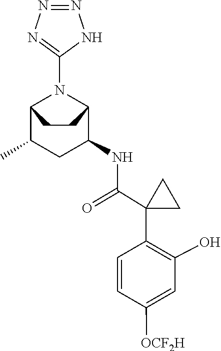 | 98C* | TA | 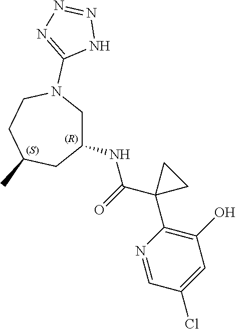 |
| | | N-((1S,2S,4S,5R)-4-methyl-8-(1H- | | | 1-(5-chloro-3-hydroxypyridin-2-yl)-N- |
| | | tetrazol-5-yl)-8- | | | ((3R,5S)-5-methyl-1-(1H-tetrazol-5- |
| | | azabicyclo[3.2.1]octan-2-yl)-1-(4- | | | yl)azepan-3-yl)cyclopropane-1- |
| | | (difluoromethoxy)-2- | | | carboxamide |
| | | hydroxyphenyl)cyclopropane-1- | | | |
| | | carboxamide | | | |
| 44A | TA | 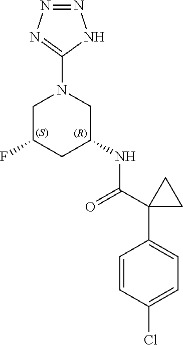 | 98D* | TA | 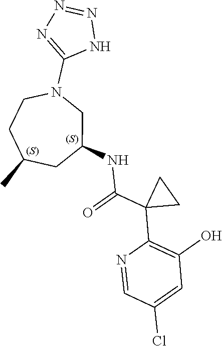 |
| | | 1-(4-chlorophenyl)-N-[(3R,5S)-5- | | | 1-(5-chloro-3-hydroxypyridin-2-yl)-N- |
| | | fluoro-1-(1H-tetrazol-5-yl)-3- | | | ((3S,5S)-5-methyl-1-(1H-tetrazol-5- |
| | | piperidyl]cyclopropanecarboxamide | | | yl)azepan-3-yl)cyclopropane-1- |
| | | | | | carboxamide |
| 44B | TA | 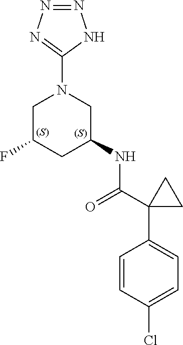 | Rac-99 | TA | 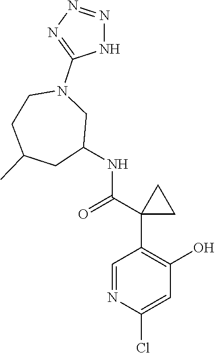 [SMILES: CC1CCN(c2nnn[nH]2)CC(NC(=O)C2(c3cnc(Cl)cc3O)CC2)C1] |
| | | 1-(4-chlorophenyl)-N-[(3S,5S)-5- | | | 1-(6-chloro-4-hydroxypyridin-3-yl)-N- |
| | | fluoro-1-(1H-tetrazol-5-yl)-3- | | | (5-methyl-1-(1H-tetrazol-5-yl)azepan-3- |
| | | piperidyl]cyclopropanecarboxamide | | | yl)cyclopropane-1-carboxamide |
| 44C | TA | 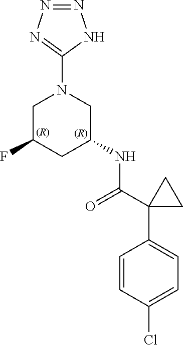 | 99A* | TA | 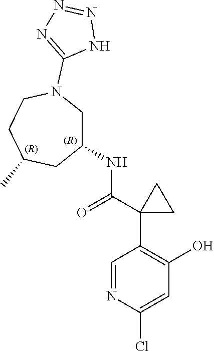 |
| | | 1-(4-chlorophenyl)-N-[(3R,5R)-5- | | | 1-(6-chloro-4-hydroxypyridin-3-yl)-N- |
| | | fluoro-1-(1H-tetrazol-5-yl)-3- | | | ((3R,5R)-5-methyl-1-(1H-tetrazol-5- |
| | | piperidyl]cyclopropanecarboxamide | | | yl)azepan-3-yl)cyclopropane-1- |
| | | | | | carboxamide |
| 44D | TA | 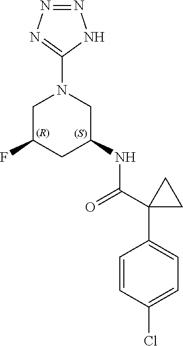 | 99B* | TA | 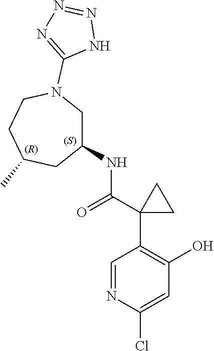 |
| | | 1-(4-chlorophenyl)-N-[(3S,5R)-5- | | | 1-(6-chloro-4-hydroxypyridin-3-yl)-N- |
| | | fluoro-1-(1H-tetrazol-5-yl)-3- | | | ((3S,5R)-5-methyl-1-(1H-tetrazol-5- |
| | | piperidyl]cyclopropanecarboxamid | | | yl)azepan-3-yl)cyclopropane-1- |
| | | | | | carboxamide |
| 45A | TA | 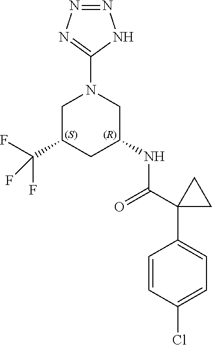 | 99C* | TA | 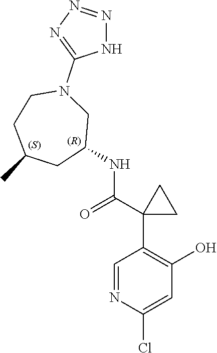 |
| | | 1-(4-chlorophenyl)-N-[(3R,5S)-1- | | | 1-(6-chloro-4-hydroxypyridin-3-yl)-N- |
| | | (1H-tetrazol-5-yl)-5- | | | ((3R,5S)-5-methyl-1-(1H-tetrazol-5- |
| | | (trifluoromethyl)-3- | | | yl)azepan-3-yl)cyclopropane-1- |
| | | piperidyl]cyclopropanecarboxamide | | | carboxamide |
| 45B | TA | 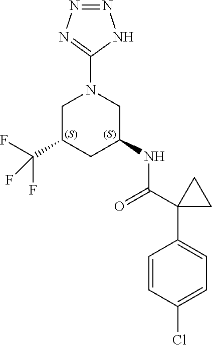 | 99D* | TA | 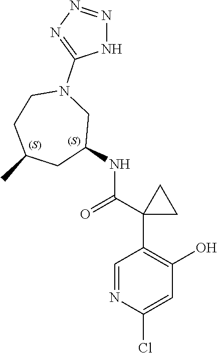 |
| | | 1-(4-chlorophenyl)-N-[(3S,5S)-1- | | | 1-(6-chloro-4-hydroxypyridin-3-yl)-N- |
| | | (1H-tetrazol-5-yl)-5- | | | ((3S,5S)-5-methyl-1-(1H-tetrazol-5- |
| | | (trifluoromethyl)-3- | | | yl)azepan-3-yl)cyclopropane-1- |
| | | piperidyl]cyclopropanecarboxamide | | | carboxamide |
| 45C | TA | 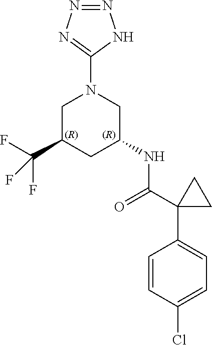 | Rac-100 | TA | 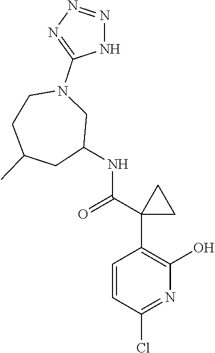 [SMILES: CC1CCN(c2nnn[nH]2)CC(NC(=O)C2(c3ccc(Cl)nc3O)CC2)C1] |
| | | 1-(4-chlorophenyl)-N-[(3R,5R)-1- | | | 1-(6-chloro-2-hydroxypyridin-3-yl)-N- |
| | | (1H-tetrazol-5-yl)-5- | | | (5-methyl-1-(1H-tetrazol-5-yl)azepan-3- |
| | | (trifluoromethyl)-3- | | | yl)cyclopropane-1-carboxamide |
| | | piperidyl]cyclopropanecarboxamide | | | |
| 45D | TA | 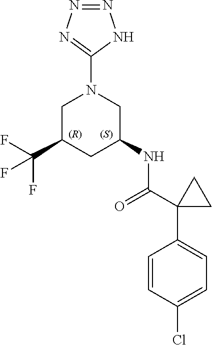 | 100A* | TA | 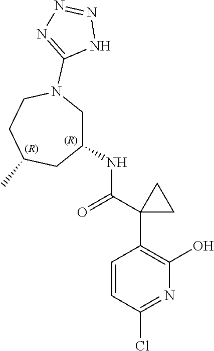 |
| | | 1-(4-chlorophenyl)-N-[(3S,5R)-1- | | | 1-(6-chloro-2-hydroxypyridin-3-yl)-N- |
| | | (1H-tetrazol-5-yl)-5- | | | ((3R,5R)-5-methyl-1-(1H-tetrazol-5- |
| | | (trifluoromethyl)-3- | | | yl)azepan-3-yl)cyclopropane-1- |
| | | piperidyl]cyclopropanecarboxamide | | | carboxamide |
| 46A | TA | 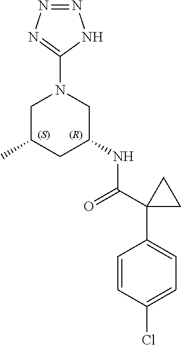 | 100B* | TA | 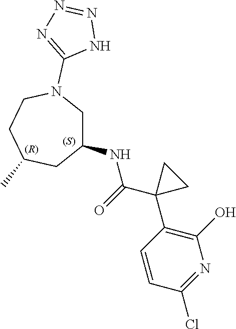 |
| | | 1-(4-chlorophenyl)-N-((3R,5S)-5- | | | 1-(6-chloro-2-hydroxypyridin-3-yl)-N- |
| | | methyl-1-(1H-tetrazol-5- | | | ((3S,5R)-5-methyl-1-(1H-tetrazol-5- |
| | | yl)piperidin-3-yl)cyclopropane-1- | | | yl)azepan-3-yl)cyclopropane-1- |
| | | carboxamide | | | carboxamide |
| 46B | TA | 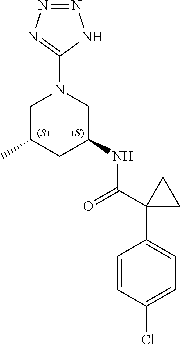 | 100C* | TA | 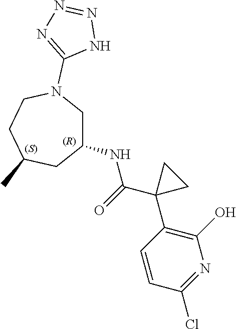 |
| | | 1-(4-chlorophenyl)-N-((3S,5S)-5- | | | 1-(6-chloro-2-hydroxypyridin-3-yl)-N- |
| | | methyl-1-(1H-tetrazol-5- | | | ((3R,5S)-5-methyl-1-(1H-tetrazol-5- |
| | | yl)piperidin-3-yl)cyclopropane-1- | | | yl)azepan-3-yl)cyclopropane-1- |
| | | carboxamide | | | carboxamide |
| 46C | TA | 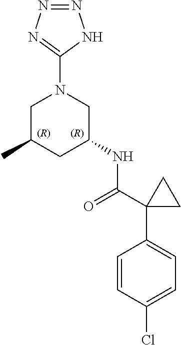 | 100D* | TA | 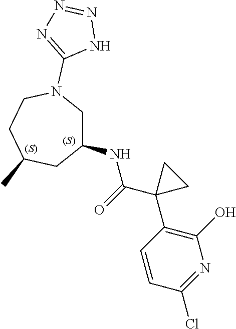 |
| | | 1-(4-chlorophenyl)-N-[(3R,5R)-5- | | | 1-(6-chloro-2-hydroxypyridin-3-yl)-N- |
| | | methyl-1-(1H-tetrazol-5-yl)-3- | | | ((3S,5S)-5-methyl-1-(1H-tetrazol-5- |
| | | piperidyl]cyclopropanecarboxamide | | | yl)azepan-3-yl)cyclopropane-1- |
| | | | | | carboxamide |
| 46D | TA | 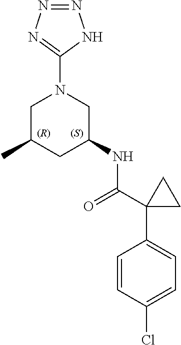 | 47C | TA | 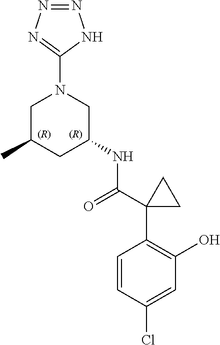 |
| | | 1-(4-chlorophenyl)-N-[(3S,5R)-5- | | | 1-(4-chloro-2-hydroxyphenyl)-N- |
| | | methyl-1-(1H-tetrazol-5-yl)-3- | | | ((3R,5R)-5-methyl-1-(1H-tetrazol-5- |
| | | piperidyl]cyclopropanecarboxamide | | | yl)piperidin-3-yl)cyclopropane-1- |
| | | | | | carboxamide |
| 47A | TA | 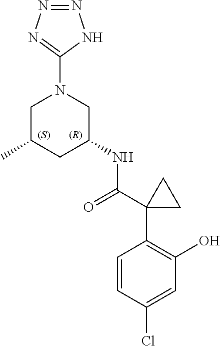 | 47D | TA | 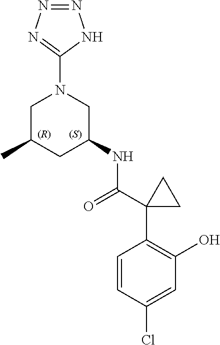 |
| | | 1-(4-chloro-2-hydroxyphenyl)-N- | | | 1-(4-chloro-2-hydroxyphenyl)-N- |
| | | ((3R,5S)-5-methyl-1-(1H-tetrazol- | | | ((3S,5R)-5-methyl-1-(1H-tetrazol-5- |
| | | 5-yl)piperidin-3-yl)cyclopropane- | | | yl)piperidin-3-yl)cyclopropane-1- |
| | | 1-carboxamide | | | carboxamide |
| 47B | TA | 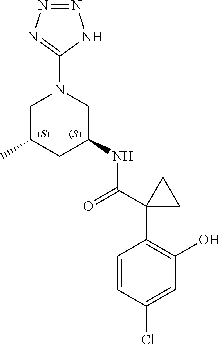 | | | |
| | | 1-(4-chloro-2-hydroxyphenyl)-N- | | | |
| | | ((3S,5S)-5-methyl-1-(1H-tetrazol- | | | |
| | | 5-yl)piperidin-3-yl)cyclopropane- | | | |
| | | 1-carboxamide | | | |

TABLE 2
Compounds of Formula (I-A)

| # | Ex | Structure/Name |
|---|---|---|
| Rac- 101 | 37 | 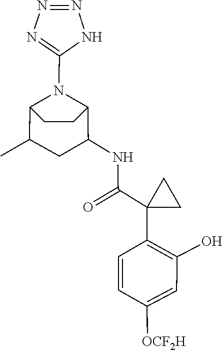 |
| | | 1-(4-(difluoromethoxy)-2- |
| | | fluorophenyl)-N-(4-methyl-8-(1H- |
| | | tetrazol-5-yl)-8- |
| | | azabicyclo[3.2.1]octan-2- |
| | | yl)cyclopropane-1-carboxamide |
| 101′* | 37 | 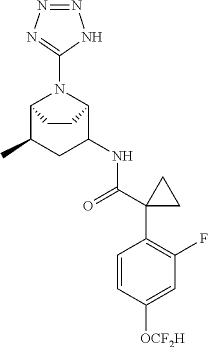 |
| | | 1-(4-(difluoromethoxy)-2- |
| | | fluorophenyl)-N-((1R,4R,5S)-4- |
| | | methyl-8-(1H-tetrazol-5-yl)-8- |
| | | azabicyclo[3.2.1]octan-2- |
| | | yl)cyclopropane-1-carboxamide |
| 101″* | 37 | 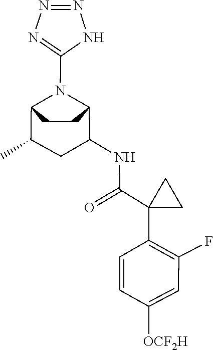 |
| | | 1-(4-(difluoromethoxy)-2- |
| | | fluorophenyl)-N-((1S,4S,5R)-4- |
| | | methyl-8-(1H-tetrazol-5-yl)-8- |
| | | azabicyclo[3.2.1]octan-2- |
| | | yl)cyclopropane-1-carboxamide |
| 101A | 37 | 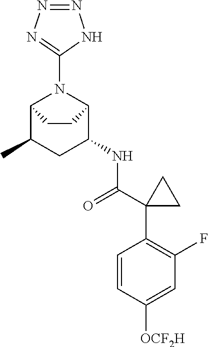 |
| | | 1-(4-(difluoromethoxy)-2- |
| | | fluorophenyl)-N-((1R,2R,4R,5S)-4- |
| | | methyl-8-(1H-tetrazol-5-yl)-8- |
| | | azabicyclo[3.2.1]octan-2- |
| | | yl)cyclopropane-1-carboxamide |
| 101B* | 37 | 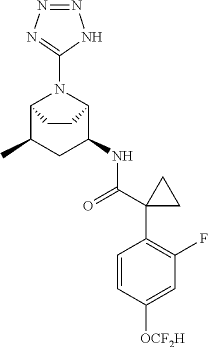 |
| | | 1-(4-(difluoromethoxy)-2- |
| | | fluorophenyl)-N-((1R,2S,4R,5S)-4- |
| | | methyl-8-(1H-tetrazol-5-yl)-8- |
| | | azabicyclo[3.2.1]octan-2- |
| | | yl)cyclopropane-1-carboxamide |
| 101C* | 37 | 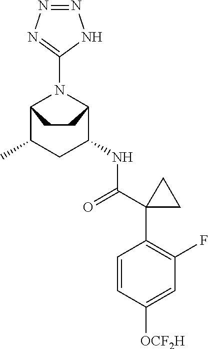 |
| | | 1-(4-(difluoromethoxy)-2- |
| | | fluorophenyl)-N-((1S,2R,4S,5R)-4- |
| | | methyl-8-(1H-tetrazol-5-yl)-8- |
| | | azabicyclo[3.2.1]octan-2- |
| | | yl)cyclopropane-1-carboxamide |
| 101D* | 37 | 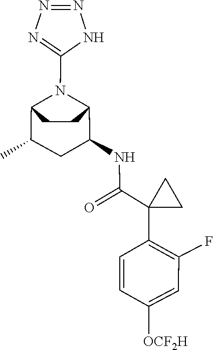 |
| | | 1-(4-(difluoromethoxy)-2- |
| | | fluorophenyl)-N-((1S,2S,4S,5R)-4- |
| | | methyl-8-(1H-tetrazol-5-yl)-8- |
| | | azabicyclo[3.2.1]octan-2- |
| | | yl)cyclopropane-1-carboxamide |

TABLE 3
Compounds of Formula (I-A)

| # | Ex | Structure/Name |
|---|---|---|
| Rac- 102 | TA | 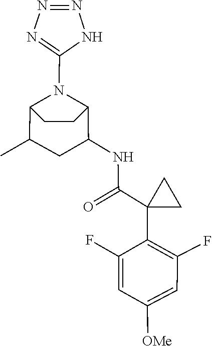 [SMILES: COc1cc(F)c(C2(C(=O)NC3CC(C)C4CCC3N4c3nnn[nH]3)CC2)c(F)c1] |
| | | 1-(2,6-difluoro-4-methoxyphenyl)-N- |
| | | (4-methyl-8-(1H-tetrazol-5-yl)-8- |
| | | azabicyclo[3.2.1]octan-2- |
| | | yl)cyclopropane-1-carboxamide |
| 102A* | 38 | 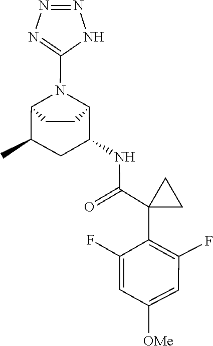 [SMILES: COc1cc(F)c(C2(C(=O)N[C@@H]3C[C@@H](C)[C@@H]4CC[C@H]3N4c3nnn[nH]3)CC2)c(F)c1] |
| | | 1-(2,6-difluoro-4-methoxyphenyl)-N- |
| | | ((1S,2R,4R,5R)-4-methyl-8-(1H- |
| | | tetrazol-5-yl)-8- |
| | | azabicyclo[3.2.1]octan-2- |
| | | yl)cyclopropane-1-carboxamide |
| 102B* | TA | 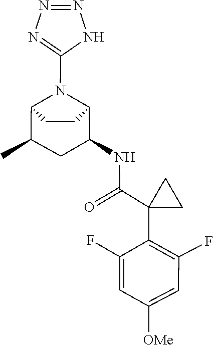 [SMILES: COc1cc(F)c(C2(C(=O)N[C@H]3C[C@@H](C)[C@@H]4CC[C@H]3N4c3nnn[nH]3)CC2)c(F)c1] |
| | | 1-(2,6-difluoro-4-methoxyphenyl)-N- |
| | | ((1S,2S,4R,5R)-4-methyl-8-(1H- |
| | | tetrazol-5-yl)-8- |
| | | azabicyclo[3.2.1]octan-2- |
| | | yl)cyclopropane-1-carboxamide |
| 102C* | TA | 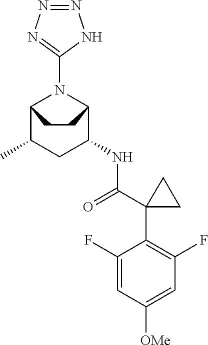 [SMILES: COc1cc(F)c(C2(C(=O)N[C@@H]3C[C@H](C)[C@H]4CC[C@@H]3N4c3nnn[nH]3)CC2)c(F)c1] |
| | | 1-(2,6-difluoro-4-methoxyphenyl)-N- |
| | | ((1S,2R,4S,5R)-4-methyl-8-(1H- |
| | | tetrazol-5-yl)-8- |
| | | azabicyclo[3.2.1]octan-2- |
| | | yl)cyclopropane-1-carboxamide |
| 102D* | 38 | 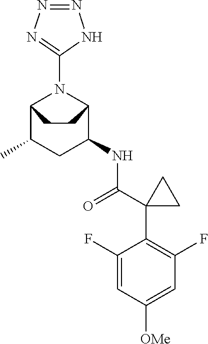 [SMILES: COc1cc(F)c(C2(C(=O)N[C@H]3C[C@H](C)[C@H]4CC[C@@H]3N4c3nnn[nH]3)CC2)c(F)c1] |
| | | 1-(2,6-difluoro-4-methoxyphenyl)-N- |
| | | ((1S,2S,4S,5R)-4-methyl-8-(1H- |
| | | tetrazol-5-yl)-8- |
| | | azabicyclo[3.2.1]octan-2- |
| | | yl)cyclopropane-1-carboxamide |
| Rac- 103 | 39 | 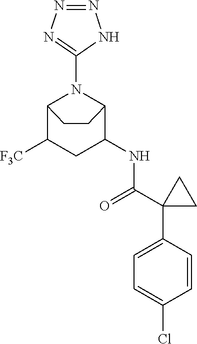 [SMILES: O=C(NC1CC(C(F)(F)F)C2CCC1N2c1nnn[nH]1)C1(c2ccc(Cl)cc2)CC1] |
| | | 1-(4-chlorophenyl)-N-(4- |
| | | trifluoromethyl-8-(1H-tetrazol-5-yl)- |
| | | 8-azabicyclo[3.2.1]octan-2- |
| | | yl)cyclopropane-1-carboxamide |
| 103′ | 39 | 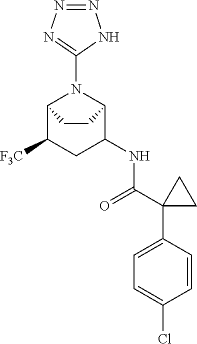 [SMILES: O=C(NC1C[C@@H](C(F)(F)F)[C@@H]2CC[C@H]1N2c1nnn[nH]1)C1(c2ccc(Cl)cc2)CC1] |
| | | N-((1R,4R,5S)-8-(1H-tetrazol-5-yl)-4- |
| | | (trifluoromethyl)-8- |
| | | azabicyclo[3.2.1]octan-2-yl)-1-(4- |
| | | chlorophenyl)cyclopropane-1- |
| | | carboxamide |
| 103″ | 39 | 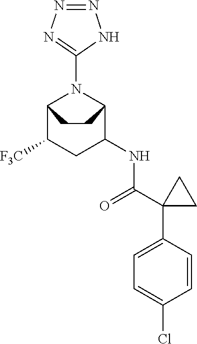 [SMILES: O=C(NC1C[C@H](C(F)(F)F)[C@H]2CC[C@@H]1N2c1nnn[nH]1)C1(c2ccc(Cl)cc2)CC1] |
| | | N-((1S,4S,5R)-8-(1H-tetrazol-5-yl)-4- |
| | | (trifluoromethyl)-8- |
| | | azabicyclo[3.2.1]octan-2-yl)-1-(4- |
| | | chlorophenyl)cyclopropane-1- |
| | | carboxamide |
| 103A* | 39 | 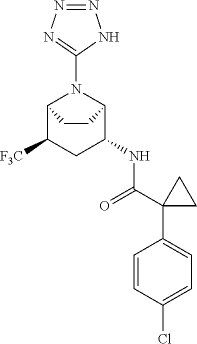 [SMILES: O=C(N[C@@H]1C[C@@H](C(F)(F)F)[C@@H]2CC[C@H]1N2c1nnn[nH]1)C1(c2ccc(Cl)cc2)CC1] |
| | | N-((1R,2R,4R,5S)-8-(1H-tetrazol-5- |
| | | yl)-4-(trifluoromethyl)-8- |
| | | azabicyclo[3.2.1]octan-2-yl)-1-(4- |
| | | chlorophenyl)cyclopropane-1- |
| | | carboxamide |
| 103B* | 39 | 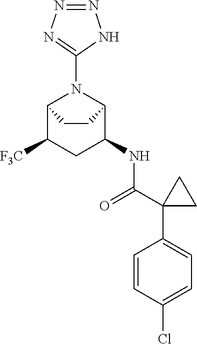 [SMILES: O=C(N[C@H]1C[C@@H](C(F)(F)F)[C@@H]2CC[C@H]1N2c1nnn[nH]1)C1(c2ccc(Cl)cc2)CC1] |
| | | N-((1R,2S,4R,5S)-8-(1H-tetrazol-5- |
| | | yl)-4-(trifluoromethyl)-8- |
| | | azabicyclo[3.2.1]octan-2-yl)-1-(4- |
| | | chlorophenyl)cyclopropane-1- |
| | | carboxamide |
| 103C* | 39 | 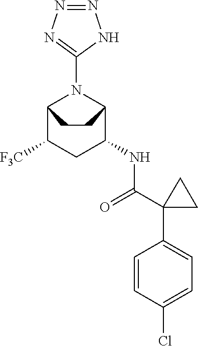 [SMILES: O=C(N[C@@H]1C[C@H](C(F)(F)F)[C@H]2CC[C@@H]1N2c1nnn[nH]1)C1(c2ccc(Cl)cc2)CC1] |
| | | N-((1S,2R,4S,5R)-8-(1H-tetrazol-5- |
| | | yl)-4-(trifluoromethyl)-8- |
| | | azabicyclo[3.2.1]octan-2-yl)-1-(4- |
| | | chlorophenyl)cyclopropane-1- |
| | | carboxamide |
| 103D* | 39 | 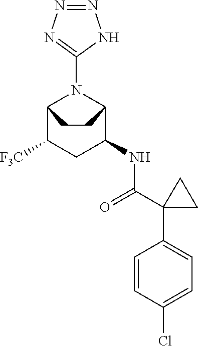 [SMILES: O=C(N[C@H]1C[C@H](C(F)(F)F)[C@H]2CC[C@@H]1N2c1nnn[nH]1)C1(c2ccc(Cl)cc2)CC1] |
| | | N-((1S,2S,4S,5R)-8-(1H-tetrazol-5- |
| | | yl)-4-(trifluoromethyl)-8- |
| | | azabicyclo[3.2.1]octan-2-yl)-1-(4- |
| | | chlorophenyl)cyclopropane-1- |
| | | carboxamide |
| Rac- 104 | 40 | 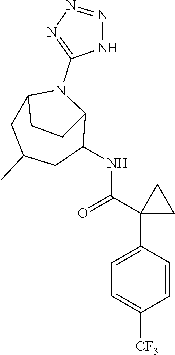 [SMILES: CC1CC(NC(=O)C2(c3ccc(C(F)(F)F)cc3)CC2)C2CCC(C1)N2c1nnn[nH]1] |
| | | N-(4-methyl-9-(1H-tetrazol-5-yl)-9- |
| | | azabicyclo[4.2.1]nonan-2-yl)-1-(4- |
| | | (trifluoromethyl)phenyl)cyclopropane- |
| | | 1-carboxamide |
| 104A* | 40 | 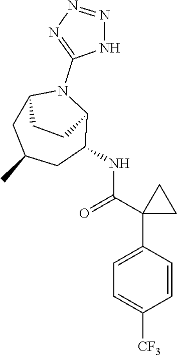 [SMILES: C[C@H]1C[C@@H]2CC[C@H]([C@H](NC(=O)C3(c4ccc(C(F)(F)F)cc4)CC3)C1)N2c1nnn[nH]1] |
| | | N-((1R,2R,4R,6S)-4-methyl-9-(1H- |
| | | tetrazol-5-yl)-9-azabicyclo[4.2.1]nonan- |
| | | 2-yl)-1-(4- |
| | | (trifluoromethyl)phenyl)cyclopropane- |
| | | 1-carboxamide |
| 104B* | 40 | 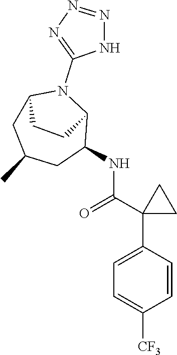 [SMILES: C[C@H]1C[C@@H]2CC[C@H]([C@@H](NC(=O)C3(c4ccc(C(F)(F)F)cc4)CC3)C1)N2c1nnn[nH]1] |
| | | N-((1R,2S,4R,6S)-4-methyl-9-(1H- |
| | | tetrazol-5-yl)-9-azabicyclo[4.2.1]nonan- |
| | | 2-yl)-1-(4- |
| | | (trifluoromethyl)phenyl)cyclopropane- |
| | | 1-carboxamide |
| 104C* | 40 | 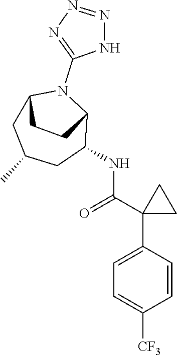 [SMILES: C[C@@H]1C[C@H]2CC[C@@H]([C@H](NC(=O)C3(c4ccc(C(F)(F)F)cc4)CC3)C1)N2c1nnn[nH]1] |
| | | N-((1S,2R,4S,6R)-4-methyl-9-(1H- |
| | | tetrazol-5-yl)-9-azabicyclo[4.2.1]nonan- |
| | | 2-yl)-1-(4- |
| | | (trifluoromethyl)phenyl)cyclopropane- |
| | | 1-carboxamide |
| 104D* | 40 | 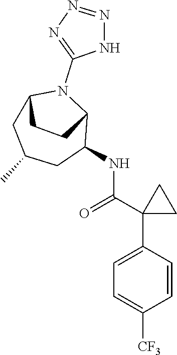 [SMILES: C[C@@H]1C[C@H]2CC[C@@H]([C@@H](NC(=O)C3(c4ccc(C(F)(F)F)cc4)CC3)C1)N2c1nnn[nH]1] |
| | | N-((1S,2S,4S,6R)-4-methyl-9-(1H- |
| | | tetrazol-5-yl)-9-azabicyclo[4.2.1]nonan- |
| | | 2-yl)-1-(4- |
| | | (trifluoromethyl)phenyl)cyclopropane- |
| | | 1-carboxamide |
| 104E* | 40 | 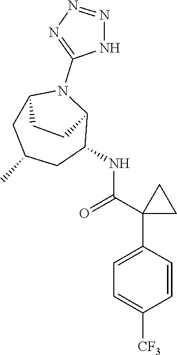 [SMILES: C[C@@H]1C[C@@H]2CC[C@H]([C@H](NC(=O)C3(c4ccc(C(F)(F)F)cc4)CC3)C1)N2c1nnn[nH]1] |
| | | N-((1R,2R,4S,6S)-4-methyl-9-(1H- |
| | | tetrazol-5-yl)-9-azabicyclo[4.2.1]nonan- |
| | | 2-yl)-1-(4- |
| | | (trifluoromethyl)phenyl)cyclopropane- |
| | | 1-carboxamide |
| 104F* | 40 | 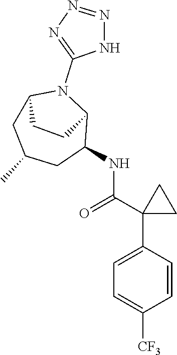 [SMILES: C[C@@H]1C[C@@H]2CC[C@H]([C@@H](NC(=O)C3(c4ccc(C(F)(F)F)cc4)CC3)C1)N2c1nnn[nH]1] |
| | | N-((1R,2S,4S,6S)-4-methyl-9-(1H- |
| | | tetrazol-5-yl)-9-azabicyclo[4.2.1]nonan- |
| | | 2-yl)-1-(4- |
| | | (trifluoromethyl)phenyl)cyclopropane- |
| | | 1-carboxamide |
| 104G* | 40 | 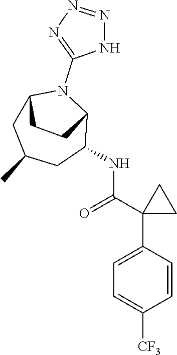 [SMILES: C[C@H]1C[C@H]2CC[C@@H]([C@H](NC(=O)C3(c4ccc(C(F)(F)F)cc4)CC3)C1)N2c1nnn[nH]1] |
| | | N-((1S,2R,4R,6R)-4-methyl-9-(1H- |
| | | tetrazol-5-yl)-9-azabicyclo[4.2.1]nonan- |
| | | 2-yl)-1-(4- |
| | | (trifluoromethyl)phenyl)cyclopropane- |
| | | 1-carboxamide |
| 104H* | 40 | 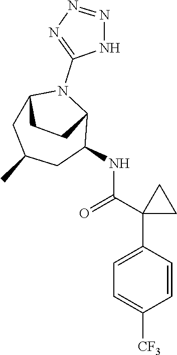 [SMILES: C[C@H]1C[C@H]2CC[C@@H]([C@@H](NC(=O)C3(c4ccc(C(F)(F)F)cc4)CC3)C1)N2c1nnn[nH]1] |
| | | N-((1S,2S,4R,6R)-4-methyl-9-(1H- |
| | | tetrazol-5-yl)-9-azabicyclo[4.2.1]nonan- |
| | | 2-yl)-1-(4- |
| | | (trifluoromethyl)phenyl)cyclopropane- |
| | | 1-carboxamide |
| 105 | TA | 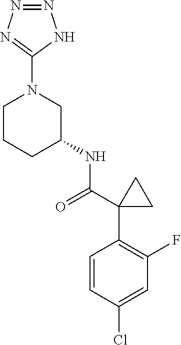 [SMILES: O=C(N[C@@H]1CCCN(c2nnn[nH]2)C1)C1(c2ccc(Cl)cc2F)CC1] |
| | | (R)-N-(1-(1H-tetrazol-5-yl)piperidin-3- |
| | | yl)-1-(4-chloro-2- |
| | | fluorophenyl)cyclopropane-1- |
| | | carboxamide |
| 106 | TA | 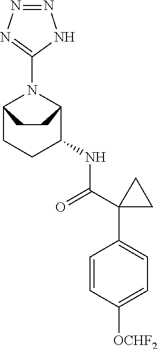 [SMILES: O=C(N[C@@H]1CC[C@H]2CC[C@@H]1N2c1nnn[nH]1)C1(c2ccc(OC(F)F)cc2)CC1] |
| | | N-((1S,2R,5S)-8-(1H-tetrazol-5-yl)-8- |
| | | azabicyclo[3.2.1]octan-2-yl)-1-(4- |
| | | (difluoromethoxy)phenyl)cyclopropane- |
| | | 1-carboxamide |

In certain embodiments, the compound is selected from the group consisting of Compound 2A*, Compound 3A*, Compound 4A*, Compound 5A*, Compound 7A*, Compound 12A*, Compound 18A*, Compound 21A*, Compound 22A*, Compound 27A*, Compound 29A*, Compound 30A*, Compound 32A*, Compound 36A*, Compound 52A, Compound 53A*, Compound 54A*, Compound 55A*, Compound 56A*, Compound 57A*, Compound 58A*, Compound 59A*, Compound 60A*, Compound 61A*, Compound 62A*, Compound 63A*, Compound 64A*, Compound 65A*, Compound 66A*, Compound 67A*, Compound 68A*, Compound 69A*, Compound 70A*, Compound 71A*, Compound 73A*, Compound 74A*, Compound 88A*, Compound 89A*, Compound 90A*, Compound 91A*, Compound 92A*, and Compound 104A*, or a pharmaceutically acceptable salt or tautomer of any of the foregoing.

In certain embodiments, the compound is selected from the group consisting of Compound 16A, Compound 17A, Compound 19A, Compound 20A, Compound 23A, Compound 33A, Compound 47A, and Compound 48A, or a pharmaceutically acceptable salt or tautomer of any of the foregoing.

In certain embodiments, the compound is selected from the group consisting of Compound 34C*, Compound 75C*, Compound 77C*, and Compound 78C*, or a pharmaceutically acceptable salt or tautomer of any of the foregoing.

In certain embodiments, the compound is selected from the group consisting of Compound 35A*, Compound 43A*, Compound 79A*, Compound 80A*, Compound 81A*, Compound 82A*, Compound 83A*, Compound 84A*, Compound 86A*, Compound 87A*, Compound 101A, Compound 102A*, and Compound 103A*, or a pharmaceutically acceptable salt or tautomer of any of the foregoing.

### (ii) Pharmaceutical Compositions

Pharmaceutical compositions comprising a compound of Formula (I-A), or a pharmaceutically acceptable salt or tautomer thereof, and a pharmaceutically acceptable carrier, are further contemplated herein.

For example, in some aspects, provided is a pharmaceutical composition comprising a compound of Formula (I-A), or a pharmaceutically acceptable salt or tautomer thereof, and a pharmaceutically acceptable carrier.

Exemplary pharmaceutical acceptable carriers may include diluents, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine.

Administration to the subject can be accomplished via any mode of administration, for example, by oral administration, topical administration, or by injection. Depending on the intended mode of administration, the pharmaceutical composition comprising the compound of Formula (I-A), or a pharmaceutically acceptable salt or tautomer thereof, can be in solid, semi-solid or liquid dosage form.

A compound of Formula (I-A), or a pharmaceutically acceptable salt or tautomer thereof, may be administered alone in the pharmaceutical composition as the sole therapeutic agent, or may be administered in combination with another therapeutic agent. Combination treatment may be achieved by way of co-administration (e.g., the two agents being administered at the same time) or sequential administration (e.g., one agent being administered first, then the other). In the case of co-administration, the compound of Formula (I-A), or a pharmaceutically acceptable salt or tautomer thereof, may be administered in the same pharmaceutical composition as the other therapeutic agent, or may be administered in a separate pharmaceutical composition. The choice of the other therapeutic agent will depend upon the diagnosis of the attending physicians and their judgment of the condition of the subject and the appropriate treatment protocol.

### (iii) Methods of Treatment

Compounds of Formula (I-A), and pharmaceutically acceptable salts and tautomers thereof, have been found useful as inhibitors of NLRP3 activity.

In some aspects, provided is a method of treating a disease or disorder in a subject in need thereof, comprising administering to the subject a compound of Formula (I-A), or a pharmaceutically acceptable salt or tautomer thereof, or a pharmaceutical composition comprising same. In some aspects, provided is a method of treating a disease or disorder in a subject in need thereof, comprising administering to the subject an effective amount of a compound of Formula (I-A), or a pharmaceutically acceptable salt or tautomer thereof, or a pharmaceutical composition comprising same. In some embodiments, the disease or disorder is associated with aberrant NLRP3 activity, and the method comprises inhibiting the aberrant NLRP3 activity such that the subject is treated.

In some embodiments, the disease or disorder is a disease or disorder of the central nervous system (CNS), a disease or disorder of the peripheral nervous system (PNS), a primary neurological disease of the muscles, an inflammatory disorder, an autoimmune disorder, cancer, an infection, obesity, a metabolic disease, a cardiovascular disease, a respiratory disease, a kidney disease, a liver disease, an ocular disease, a skin disease, a lymphatic disease, a rheumatic disease, a psychological disease, graft versus host disease, pain (including disorders related to pain management), or an NLRP3-related disease in a subject that has been determined to carry a germline or somatic non-silent mutation in NLRP3.

In some embodiments, the disease or disorder is a disease or disorder of central nervous system and/or peripheral nervous system (“PNS”), such as dementia, Alzheimer's disease (“AD”) epilepsy, traumatic brain injury (“TBI”), multiple sclerosis (“MS”), a developmental disturbance, acute disseminated encephalopathy, transverse myelitis, Parkinson's disease (“PD”), amyotrophic lateral sclerosis (“ALS”), Huntington's disease (“HD”), spinal cord injury, or obesity related to neuroinflammation.

In some embodiments, the disease or disorder is a primary neurological disease of the muscle, such as a dystrophy or spinal muscular atrophy.

In some embodiments, the disease or disorder is an inflammatory disorder, such as gout or anemia of inflammation.

In some embodiments, the disease or disorder is an autoimmune disease, such as ulcerative colitis.

In some embodiments, the disease or disorder is cancer, such as skin cancer or colon cancer.

In some embodiments, the disease or disorder is an infection, such as a neuro-infection.

In some embodiments, the disease or disorder is a metabolic disease, such as diabetes, e.g., type 2 diabetes.

In some embodiments, the disease or disorder is obesity. In some embodiments, the obesity is related to neuroinflammation, e.g., hypothalamic inflammation and/or gliosis. In some embodiments, the obesity is related to a metabolic disorder.

In some embodiments, the disease or disorder is a cardiovascular disease, such as stroke, atherosclerosis or atherosclerotic cardiovascular disease (ASCVD).

In some embodiments, the disease or disorder is a respiratory disease, such as asthma (e.g., steroid-resistant asthma, severe steroid-resistant asthma) or chronic obstructive pulmonary disease (“COPD”).

In some embodiments, the disease or disorder is a kidney disease, such as acute kidney disease, a chronic kidney disease, or a rare kidney disease. In certain embodiments, the chronic kidney disease is chronic kidney failure.

In some embodiments, the disease or disorder is a liver disease, such as nonalcoholic fatty liver disease (NAFLD) or non-alcoholic steatohepatitis (NASH, also known as MASH or metabolic dysfunction-associated steatohepatitis).

In some embodiments, the disease or disorder is an ocular disease, such as optic neuritis or macular degeneration.

In some embodiments, the disease or disorder is a skin disease, such as psoriasis, hidradenitis suppurativa (HS), or atopic dermatitis.

In some embodiments, the disease or disorder is a lymphatic disease.

In some embodiments, the disease or disorder is a rheumatic disease, such as osteoarthritis, dermatomyositis, Still's disease, or juvenile idiopathic arthritis.

In some embodiments, the disease or disorder is a psychological disease, such as a neuropsychiatric condition, including depression, major depressive disorder, or refractory depression.

In some embodiments, the disease or disorder is a graft versus host disease.

In some embodiments, the disease or disorder is pain (including disorders related to pain management), such as pain management addiction, osteoarthritis pain, or allodynia.

In some embodiments, the NLRP3-related disease in a subject that has been determined to carry a germline or somatic non-silent mutation in NLRP3 is cryopyrin-associated autoinflammatory syndrome.

In some embodiments, the cryopyrin-associated autoinflammatory syndrome is familial cold autoinflammatory syndrome, Muckle-Wells syndrome, or neonatal onset multisystem inflammatory disease (NOMID).

In some embodiments, the disease or disorder is dementia, Alzheimer's disease (“AD”), epilepsy, traumatic brain injury (“TBI”), multiple sclerosis (“MS”), developmental disturbances, acute disseminated encephalopathy, transverse myelitis, Parkinson's disease (“PD”), amyotrophic lateral sclerosis (“ALS”), spinal muscular atrophy, Huntington's disease (“HD”), a spinal cord injury, a dystrophy, a neuro-infection, a pain management addiction, a neuropsychiatric condition (e.g. depression, major depressive disorder, refractory depression), neonatal onset multisystem inflammatory disease (“NOMID”), asthma, osteoarthritis, ulcerative colitis, gout, anemia of inflammation, Still's disease, chronic obstructive pulmonary disease (“COPD”), osteoarthritis pain, hidradenitis suppurativa, or obesity related to neuroinflammation.

In other aspects, provided is a method of modulating (e.g., inhibiting) NLRP3 activity (e.g., in vitro or in vivo in a cell, or in a subject), comprising contacting the cell with or administering to the subject a compound of Formula (I-A), or a pharmaceutically acceptable salt or tautomer thereof. In certain the compound or a pharmaceutically acceptable salt or tautomer thereof is administered to the cell or subject in an effective amount.

### (iv) Methods of Preparation

Compounds of Formula (I-A), and salts and tautomers thereof, may be synthesized following General Schemes A-C, as provided below. The Examples further described non-limiting examples of this general syntheses.

### General Method, Protocol A

A suitable general route for the preparation of compounds described herein follows Protocol A as depicted in General Scheme A.

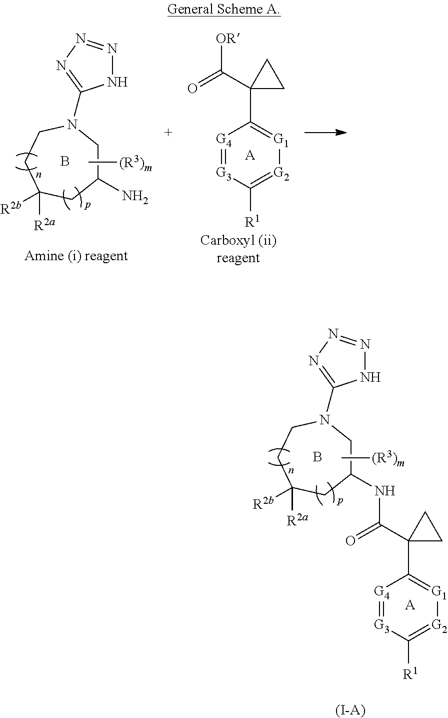

The reaction involves peptide coupling of an amine (i) reagent, or a salt or tautomer thereof, with a carboxyl (ii) reagent, or salt thereof, wherein R′ is hydrogen or an oxygen protecting group, to provide a compound of Formula (I-A), or a salt or tautomer thereof.

### General Method, Protocol B

Another suitable general route for the preparation of compounds described herein follows Protocol B as depicted in General Scheme B.

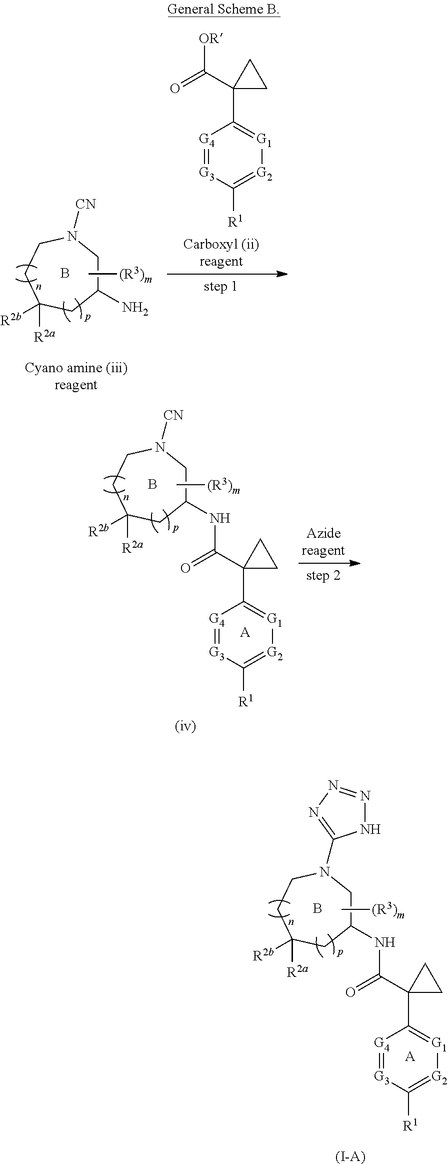

Step 1 involves reaction of a cyano amine (iii) reagent, or a salt thereof, with a carboxyl (ii) reagent, or a salt thereof, wherein R′ is hydrogen or an oxygen protecting group, to provide a cyano amide (iv) intermediate, or a salt thereof. Step 2 involves subsequent treatment of the cyano amide (iv) intermediate, or a salt thereof, with an azide reagent, such as NaN₃ or trimethylsilyl azide (TMS-N₃), to provide a compound of Formula (I-A), or a salt or tautomer thereof.

### General Method, Protocol C

Another suitable general route for the preparation of compounds described herein follows Protocol C as depicted in General Scheme C.

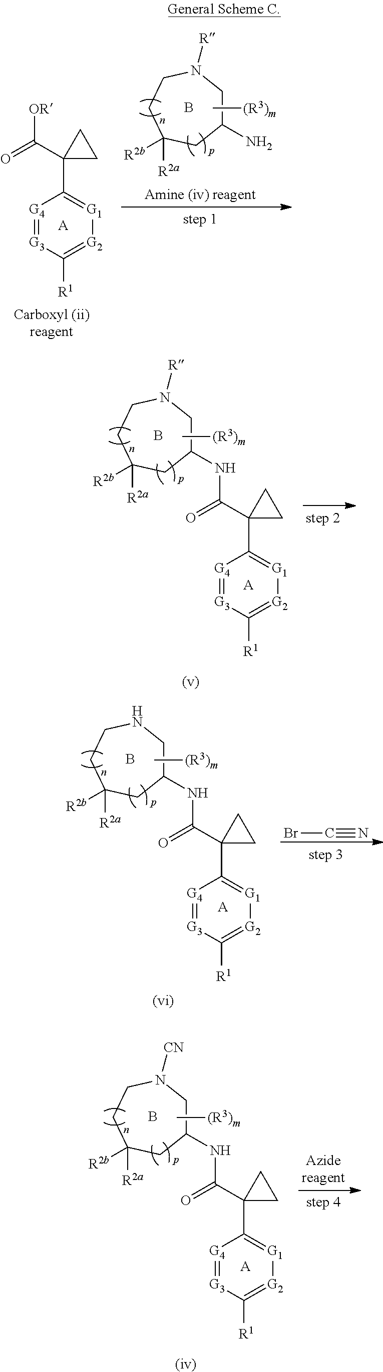

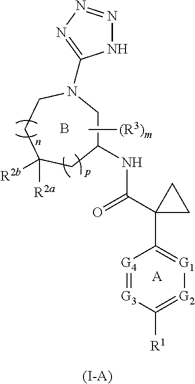

Step 1 involves peptide coupling of a carboxyl (ii) reagent, or a salt thereof, wherein R′ is hydrogen or an oxygen protecting group, with a protected amine (iv) reagent, or a salt thereof, wherein R″ is an amine protecting group, to provide the protected amide (v) intermediate, or a salt thereof. Step 2 involves deprotection of the protected amide (v) intermediate, or a salt thereof, to provide the deprotected amide (vi) intermediate, or salt thereof. Step 3 involves treatment of the deprotected amide (vi) intermediate, or a salt thereof, with cyanobromide to provide a cyano amide (iv) intermediate, or a salt thereof. Step 4 involves treatment of the cyano amide (iv) intermediate, or a salt thereof with an azide reagent, such as NaN₃ or trimethylsilyl azide (TMS-N₃), to provide a compound of Formula (I-A), or a salt or tautomer thereof.

### (v) Biological Assays

Various in vitro or in vivo biological assays may be suitable for detecting the effect of the compounds of the present disclosure. These in vitro or in vivo biological assays can include, but are not limited to, binding assays, cellular assays (cell lines, primary cells and whole blood), in vitro cell viability assays, as well as assays for determining NLRP3 potency, unbound clearance, solubility, and permeability.

In some embodiments, the compounds of the instant disclosure may be tested for their human-NLRP3 inhibitory activity/potency using known procedures, such as the methodology reported in Coll et al. Nat Med. (2015) 21(3):248-255. See also the Examples, Biological Assay Methods section.

In some embodiments, the compounds of the instant disclosure may be tested for unbound clearance (Clu) following known procedures, such as described in Miller et al., J Med. Chem. (2020) 63:12156-12170. For example, unbound clearance (Clu) may be calculated by dividing total clearance (‘CL’ in mL/min/kg) as measured in blood or plasma by the unbound fraction in plasma (fu).

In some embodiments, the solubility of compounds of the instant disclosure may be determined following known procedures, such as described in Alsenz and Kansy, Advanced Drug Delivery Reviews (2007) 59:546-567, and Wang et al. J Mass Spectrom. (2000) 35:71-76. For example, the kinetic solubility in physiologically relevant media may be measured using serial dilution and two hour incubation period, followed by filtration, and reported in uM by LC-MS/MS. Thermodynamic solubility in physiologically relevant media may be measured by LC-MS/MS, after a twenty-four hour incubation, followed by filtration, and reported in mg/mL.

### (vi) Exemplary Embodiments

Additional Exemplary Embodiments are as set forth below. Other embodiments are contemplated in the claims.

Exemplary Embodiment 1. A compound of Formula (I-A):

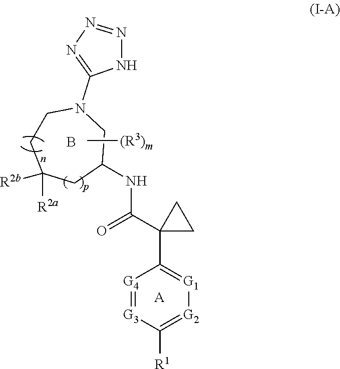

or a pharmaceutically acceptable salt or tautomer thereof;
wherein:
Ring A is a ring system wherein:
   G₁ is CR^{G1} or N; G₂ is CR^{G2} or N; G₃ is CR^{G3} or N; and G₄ is CR^{G4} or N; provided no more than two of G₁, G₂, G₃, and G₄ are N;
   R¹ is halo, C₁₋₆ alkyl, C₁₋₆ haloalkyl, —OR^{G5}, —SR^{G5}, —N(R^{G5})₂, C₃-C₄ carbocyclyl, or 3-4 membered heterocyclyl, wherein the carbocyclyl and heterocyclyl are independently substituted with 0, 1, 2, or 3 halo, C₁₋₆ alkyl, C₁₋₆haloalkyl, —OR^{G5}, —SR^{G5}, or —N(R^{G5})₂
   or R¹ and G₂, together with the atoms to which they are attached, are joined to form a 5-membered heteroaryl ring independently substituted with 0, 1, 2, or 3;
   R^{G1}, R^{G2}, R^{G3}, and R^{G4} are each independently selected from the group consisting of hydrogen, halo, C₁₋₆alkyl, 6 haloalkyl, and —OR^{G6}; and
   R^{G5} and R^{G6} are each independently hydrogen, C₁₋₆ alkyl, or C₁₋₆ haloalkyl; each instance of R^{G7} is independently halo, C₁₋₆ alkyl, C₁₋₆ haloalkyl, —OR^{G5}, —SR^{G5}, and —N(R^{G5})₂; and
Ring B is a ring system wherein:
   n is 0 or 1;
   p is 1 or 2;
   m is 0, 1, 2, or 3;
   each instance of R^{2a} and R^{2b} is independently hydrogen, halo, C₁₋₆ alkyl, C₁₋₆ haloalkyl, C₃-C₄ carbocyclyl, or 3-4 membered heterocyclyl, wherein the carbocyclyl or heterocyclyl are each independently substituted with 0, 1, 2, or 3 halo, or R^{2a} and R^{2b} are joined to form a C₃ carbocyclyl independently substituted with 0, 1, 2, or 3 halo; and
   each instance of R³ is independently halo, C₁₋₆ alkyl or C₁₋₆ haloalkyl, or two R³ groups may be joined to form a C₁₋₃ alkylene bridging group or C₁₋₃ haloalkylene bridging group between the two atoms to which they are attached.

Exemplary Embodiment 2. The compound of Exemplary Embodiment 1, wherein the compound is of Formula:

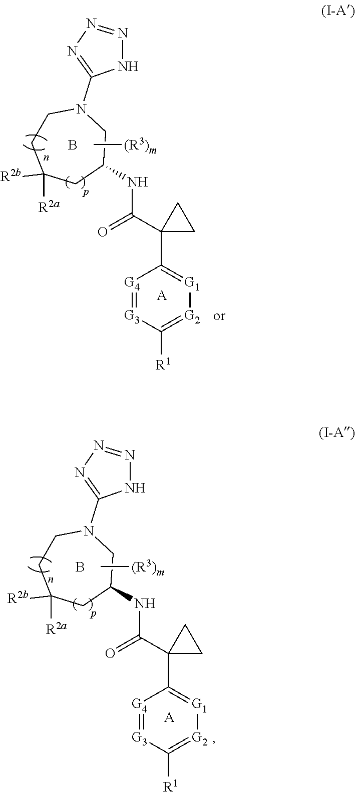

or a pharmaceutically acceptable salt or tautomer thereof.

Exemplary Embodiment 3. The compound of Exemplary Embodiment 2, wherein the compound is of Formula:

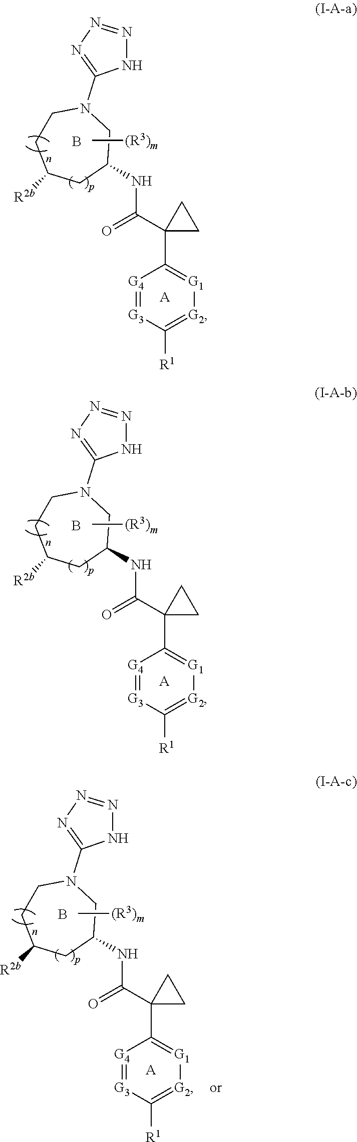

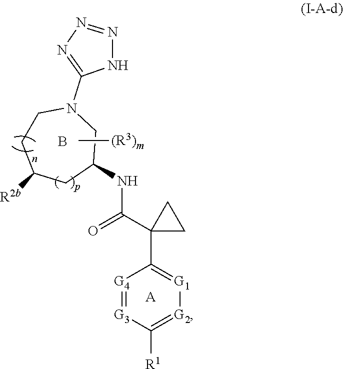

or a pharmaceutically acceptable salt or tautomer thereof.

Exemplary Embodiment 4. The compound of Exemplary Embodiment 1, wherein the compound is of the Formula:

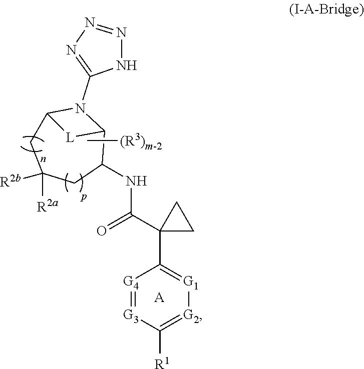

or a pharmaceutically acceptable salt or tautomer thereof, wherein L is a C₁₋₃ alkylene bridging group or a C₁₋₃ haloalkylene bridging group.

Exemplary Embodiment 5. The compound of Exemplary Embodiment 4, wherein the compound is of the Formula:

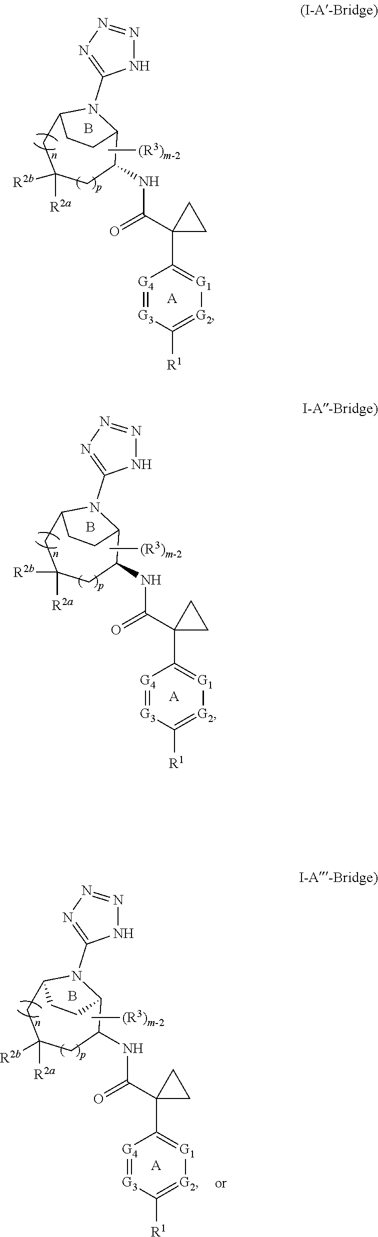

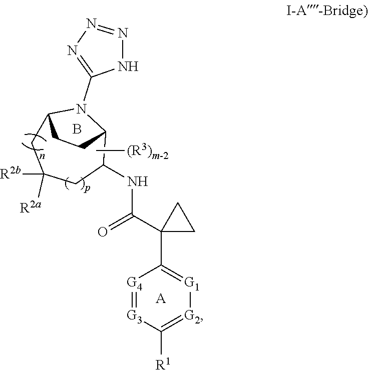

or a pharmaceutically acceptable salt or tautomer thereof, wherein L is a C₁₋₃ alkylene bridging group or a C₁₋₃ haloalkylene bridging group.

Exemplary Embodiment 6. The compound of Exemplary Embodiment 4, wherein the compound is of the Formula:

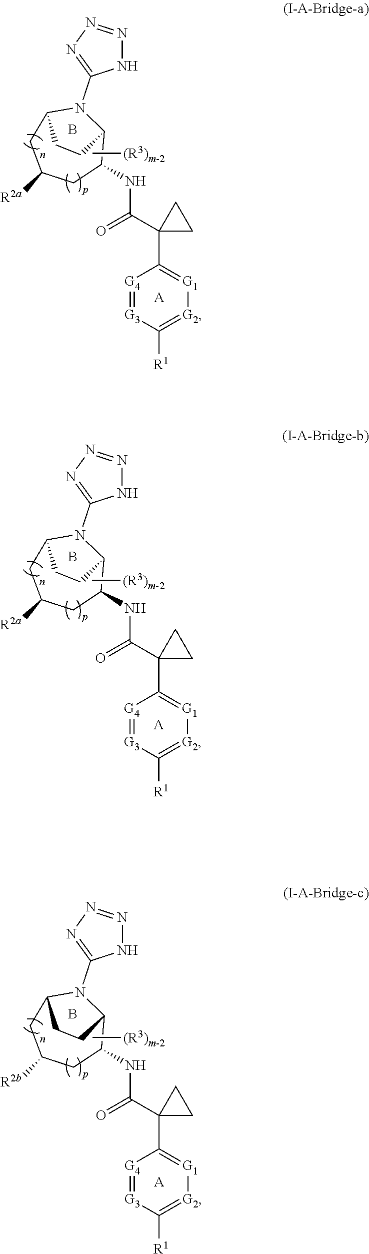

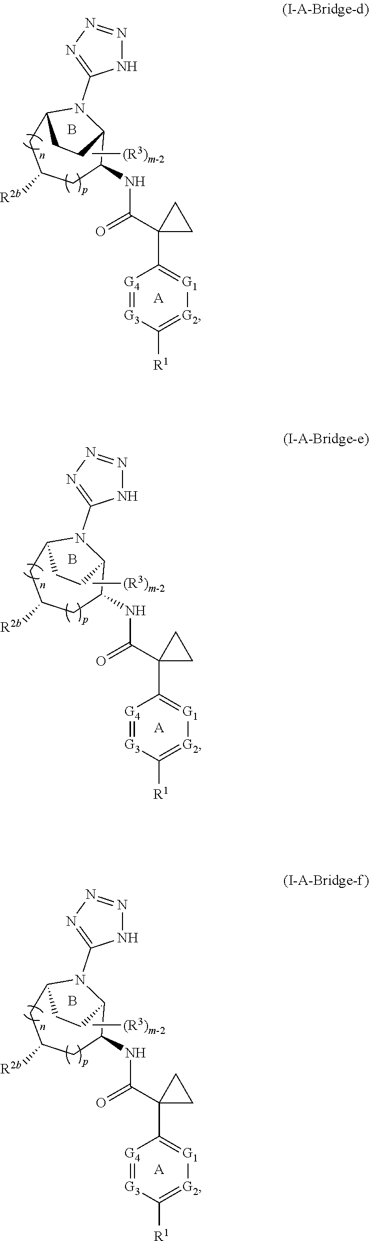

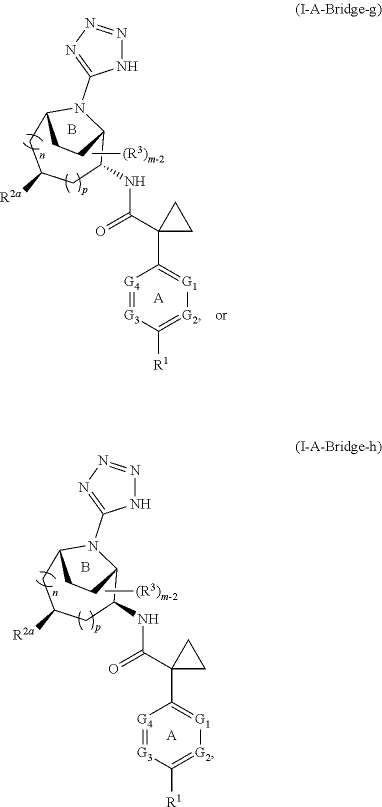

or a pharmaceutically acceptable salt or tautomer thereof.

Exemplary Embodiment 7. The compound of any one of Exemplary Embodiments 1-6, or a pharmaceutically acceptable salt or tautomer thereof, wherein Ring A is a ring system wherein:
R¹ is halo, C₁₋₆ alkyl, C₁₋₆ haloalkyl, —OR^{G}, —SR^{G}, C₃-C₄ carbocyclyl, wherein the carbocyclyl is independently substituted with 0, 1, 2, or 3 halo, C₁₋₆ alkyl, C₁₋₆ haloalkyl, —OR^{G5}, —SR^{G5}, or —N(R^{G5})₂
R^{G1}, R^{G2}, R^{G3}, and R^{G4} are each independently selected from the group consisting of hydrogen, halo, C₁₋₆ alkyl, and —OR^{G6}; and
R^{G5} and R^{G6} are each independently hydrogen, C₁₋₆ alkyl, or C₁₋₆ haloalkyl.

Exemplary Embodiment 8. The compound of Exemplary Embodiment 7, or a pharmaceutically acceptable salt or tautomer thereof, wherein Ring A is a ring system wherein:
R¹ is Cl, Br, methyl, isopropyl, cyclopropyl, difluorocyclopropyl, cyclobutyl, OCF₂H, OCF₃, CF₂H, CF₃, SCF₃, or SCF₂H; and
R^{G1}, R^{G2}, R^{G3}, and R^{G4} are each independently selected from the group consisting of hydrogen, F, Cl, methyl, OH, OCH₃, and OCF₂H.

Exemplary Embodiment 9. The compound of any one of Exemplary Embodiments 1-8, or a pharmaceutically acceptable salt or tautomer thereof, wherein Ring B is a ring system wherein:
each instance of R^{2a} and R^{2b} is independently hydrogen, halo, C₁₋₆ alkyl, C₁₋₆ haloalkyl, or C₃-C₄ carbocyclyl; and
each instance of R³ is independently C₁₋₆ alkyl, or two R³ groups may be joined to form a C₁₋₃ alkylene bridging group between the two atoms to which they are attached.

Exemplary Embodiment 10. The compound of Exemplary Embodiment 9, or a pharmaceutically acceptable salt or tautomer thereof, wherein Ring B is a ring system wherein:
each instance of R^{2a} and R^{2b} is independently hydrogen, F, CF₃, methyl or cyclopropyl; and
each instance of R³ is independently methyl, or two R³ groups may be joined to form an ethylene bridging group between the two atoms to which they are attached.

Exemplary Embodiment 11. The compound of any one of Exemplary Embodiments 1-10, or a pharmaceutically acceptable salt or tautomer thereof, wherein G₁ is CR^{G1}, G₂ is CR^{G2}, G₃ is CR^{G3}, and G₄ is CR^{G4}.

Exemplary Embodiment 12. The compound of Exemplary Embodiment 11, or a pharmaceutically acceptable salt or tautomer thereof, wherein G₁ is CH, G₂ is CH, G₃ is CH, and G₄ is CH.

Exemplary Embodiment 13. The compound of Exemplary Embodiment 11, or a pharmaceutically acceptable salt or tautomer thereof, wherein G₁ is CR^{G1}, G₂ is CH, G₃ is CH, and G₄ is CH.

Exemplary Embodiment 14. The compound of Exemplary Embodiment 11, or a pharmaceutically acceptable salt or tautomer thereof, wherein G₁ is CH, G₂ is CR^{G2}, G₃ is CH, and G₄ is CH.

Exemplary Embodiment 15. The compound of Exemplary Embodiment 11, or a pharmaceutically acceptable salt or tautomer thereof, wherein G₁ is CR^{G1}, G₂ is CR^{G2}, G₃ is CH, and G₄ is CH.

Exemplary Embodiment 16. The compound of Exemplary Embodiment 11, or a pharmaceutically acceptable salt or tautomer thereof, wherein G₁ is CR^{G1}, G₂ is CH, G₃ is CR^{G3}, and G₄ is CH.

Exemplary Embodiment 17. The compound of any one of Exemplary Embodiments 1-10, or a pharmaceutically acceptable salt or tautomer thereof, wherein G₁ is CR^{G1}, G₂ is N, G₃ is CR^{G3}, and G₄ is CR^{G4}.

Exemplary Embodiment 18. The compound of Exemplary Embodiment 17, or a pharmaceutically acceptable salt or tautomer thereof, wherein G₁ is CR^{G1}, G₂ is N, G₃ is CH, and G₄ is CH.

Exemplary Embodiment 19. The compound of any one of Exemplary Embodiments 1-10, or a pharmaceutically acceptable salt or tautomer thereof, wherein G₁ is CR^{G1}, G₂ is CR^{G2}, G₃ is N, and G₄ is CR^{G4}.

Exemplary Embodiment 20. The compound of Exemplary Embodiment 19, or a pharmaceutically acceptable salt or tautomer thereof, wherein G₁ is CR^{G1}, G₂ is CH, G₃ is N, and G₄ is CH.

Exemplary Embodiment 21. The compound of any one of Exemplary Embodiments 1-10, or a pharmaceutically acceptable salt t or tautomer hereof, wherein G₁ is CR^{G1}, G₂ is CR^{G2}, G₃ is CR^{G3}, and G₄ is N.

Exemplary Embodiment 22. The compound of Exemplary Embodiment 21, or a pharmaceutically acceptable salt or tautomer thereof, wherein G₁ is CR^{G1}, G₂ is CH, G₃ is CH, and G₄ is N.

Exemplary Embodiment 23. The compound of any one of Exemplary Embodiments 1-22, or a pharmaceutically acceptable salt or tautomer thereof, wherein R¹ is halo, C₁₋₆ alkyl, C₁₋₆ haloalkyl, —OR^{G5}, —SR^{G5}, C₃-C₄ carbocyclyl, wherein the carbocyclyl is independently substituted with 0, 1, 2, or 3 halo, C₁₋₆ alkyl, C₁₋₆haloalkyl, —OR^{G5}, —SR^{G5}, or —N(R^{G5})₂.

Exemplary Embodiment 24. The compound of Exemplary Embodiment 23, or a pharmaceutically acceptable salt or tautomer thereof, wherein R¹ is Cl, Br, methyl, isopropyl, cyclopropyl, difluorocyclopropyl, cyclobutyl, OCF₂H, OCF₃, CF₂H, CF₃, SCF₃, or SCF₂H.

Exemplary Embodiment 25. The compound of any one of Exemplary Embodiments 1-22, or a pharmaceutically acceptable salt or tautomer thereof, wherein R^{G1}, R^{G2}, R^{G3}, and R^{G4} are each independently selected from the group consisting of hydrogen, halo, C₁₋₆ alkyl, and —OR^{G6}.

Exemplary Embodiment 26. The compound of Exemplary Embodiment 25, or a pharmaceutically acceptable salt or tautomer thereof, wherein R^{G1}, R^{G2}, R^{G3}, and R^{G4} are each independently selected from the group consisting of hydrogen, F, Cl, methyl, OH, OCH₃, and OCF₂H.

Exemplary Embodiment 27. The compound of any one of Exemplary Embodiments 1-22, or a pharmaceutically acceptable salt or tautomer thereof, wherein R^{G5} and R^{G6} are each independently hydrogen, C₁₋₆alkyl, or C₁₋₆haloalkyl.

Exemplary Embodiment 28. The compound of Exemplary Embodiment 27, or a pharmaceutically acceptable salt or tautomer thereof, wherein R^{G5} is CF₂H or CF₃.

Exemplary Embodiment 29. The compound of Exemplary Embodiment 27, or a pharmaceutically acceptable salt or tautomer thereof, wherein R^{G6} is hydrogen, methyl or CF₂H.

Exemplary Embodiment 30. The compound of any one of Exemplary Embodiment 1-29, or a pharmaceutically acceptable salt or tautomer thereof, wherein each instance of R^{2a} and R^{2b} is independently hydrogen, halo, C₁₋₆ alkyl, C₁₋₆ haloalkyl, or C₃-C₄ carbocyclyl.

Exemplary Embodiment 31. The compound of Exemplary Embodiment 30, or a pharmaceutically acceptable salt or tautomer thereof, wherein each instance of R^{2a} and R^{2b} is independently hydrogen, F, CF₃, methyl or cyclopropyl.

Exemplary Embodiment 32. The compound of any one of Exemplary Embodiments 1-31, or a pharmaceutically acceptable salt or tautomer thereof, wherein each instance of R³ is independently C₁₋₆ alkyl.

Exemplary Embodiment 33. The compound of Exemplary Embodiment 32, or a pharmaceutically acceptable salt or tautomer thereof, wherein each instance of R³ is independently methyl.

Exemplary Embodiment 34. The compound of any one of Exemplary Embodiments 1-31, or a pharmaceutically acceptable salt or tautomer thereof, wherein two R³ groups may be joined to form a C₁₋₃ alkylene bridging group between the two atoms to which they are attached.

Exemplary Embodiment 35. The compound of Exemplary Embodiment 34, or a pharmaceutically acceptable salt or tautomer thereof, wherein two R³ groups may be joined to form an ethylene bridging group between the two atoms to which they are attached.

Exemplary Embodiment 36. The compound of any one of Exemplary Embodiments 1-35, or a pharmaceutically acceptable salt or tautomer thereof, wherein n is 0.

Exemplary Embodiment 37. The compound of any one of Exemplary Embodiments 1-35, or a pharmaceutically acceptable salt or tautomer thereof, wherein n is 1.

Exemplary Embodiment 38. The compound of any one of Exemplary Embodiments 1-37, or a pharmaceutically acceptable salt or tautomer thereof, wherein p is 1.

Exemplary Embodiment 39. The compound of any one of Exemplary Embodiments 1-37, or a pharmaceutically acceptable salt or tautomer thereof, wherein p is 2.

Exemplary Embodiment 40. The compound of any one of Exemplary Embodiments 1-39, or a pharmaceutically acceptable salt or tautomer thereof, wherein m is 0.

Exemplary Embodiment 41. The compound of any one of Exemplary Embodiments 1-39, or a pharmaceutically acceptable salt or tautomer thereof, wherein m is 1.

Exemplary Embodiment 42. The compound of any one of Exemplary Embodiments 1-39, or a pharmaceutically acceptable salt or tautomer thereof, wherein m is 2.

Exemplary Embodiment 43. The compound of any one of Exemplary Embodiments 1-42, or a pharmaceutically acceptable salt or tautomer thereof, wherein Ring A of formula:

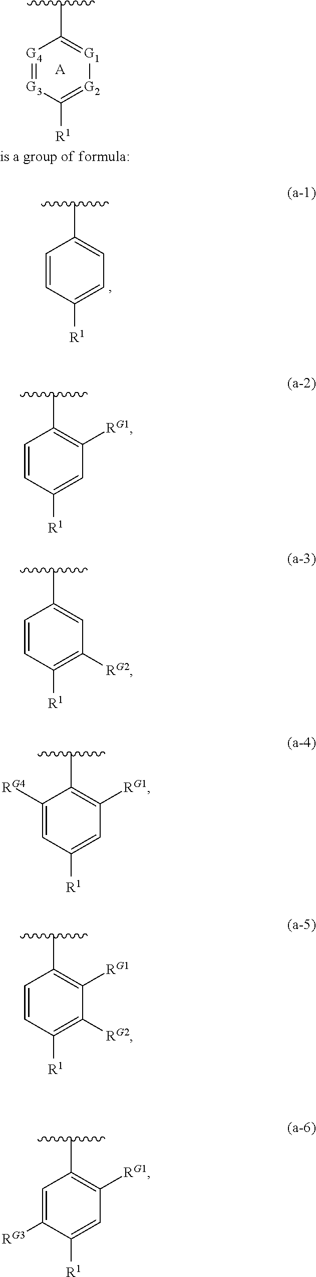

wherein R^{G1}, R^{G2}, R^{G3}, and R^{G4} are each independently selected from the group consisting of halo, C₁₋₆ alkyl, C₆₋₆haloalkyl, and —OR^{G6}.

Exemplary Embodiment 44. The compound of Exemplary Embodiment 43, or a pharmaceutically acceptable salt or tautomer thereof, wherein Ring A of formula (a-2), (a-4), (a-5), or (a-6) is a group of formula:

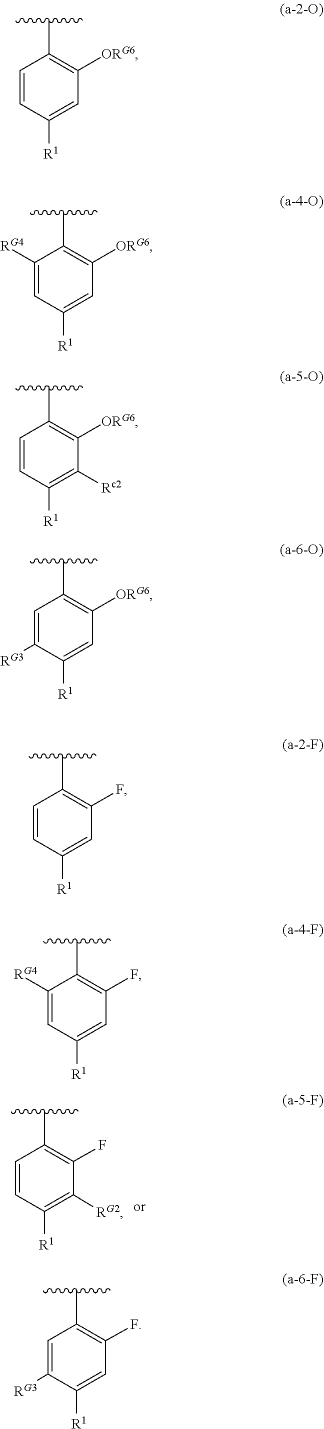

Exemplary Embodiment 45. The compound of any one of Exemplary Embodiments 1-42, or a pharmaceutically acceptable salt or tautomer thereof, wherein Ring A of formula:

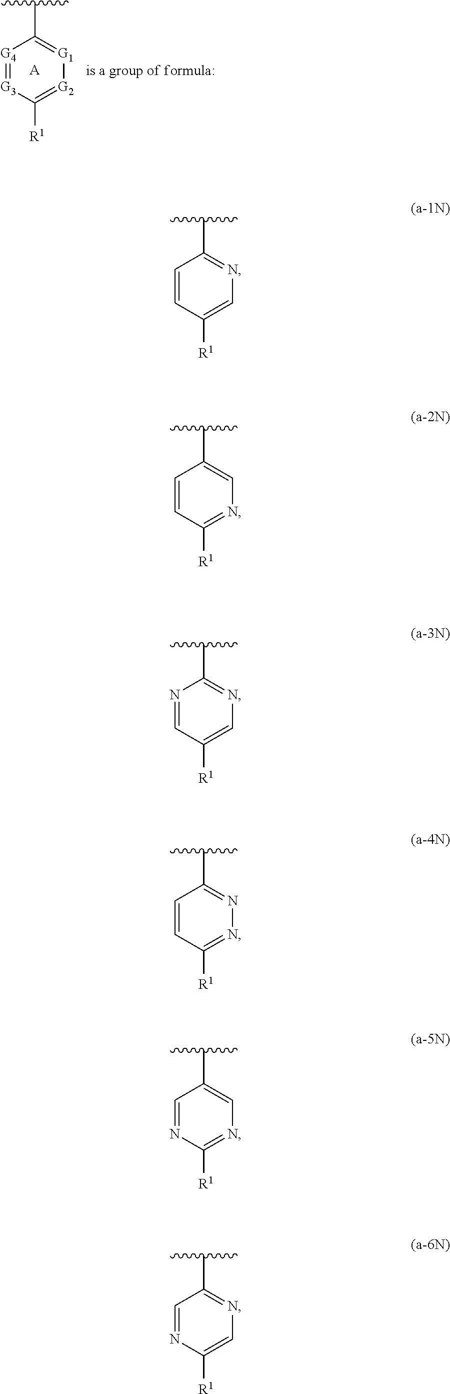

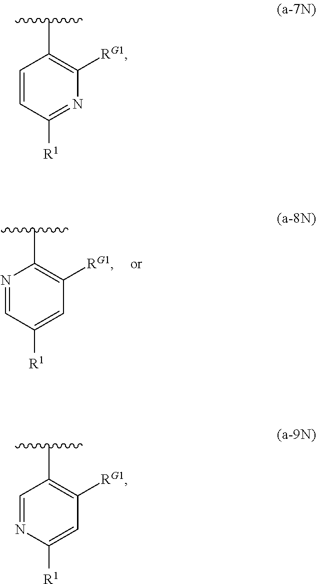

wherein R^{G1} is halo, C₁₋₆ alkyl, C₁₋₆haloalkyl, or —OR^{G6}.

Exemplary Embodiment 46. The compound of Exemplary Embodiment 45, or a pharmaceutically acceptable salt or tautomer thereof, wherein Ring A of formula (a-7N), (a-8N), or (a-9N) is of the formula:

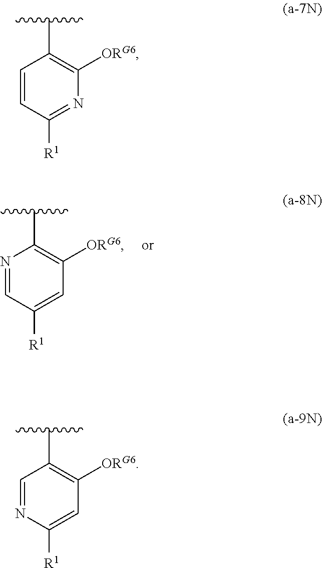

Exemplary Embodiment 47. The compound of any one of Exemplary Embodiments 1-42, or a pharmaceutically acceptable salt or tautomer thereof, wherein Ring A of formula:

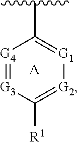

and R¹ and G₂, together with the atoms to which they are attached, are joined to form a 5-membered heteroaryl ring, wherein the Ring A, R¹, and G₂ provide a group of formula:

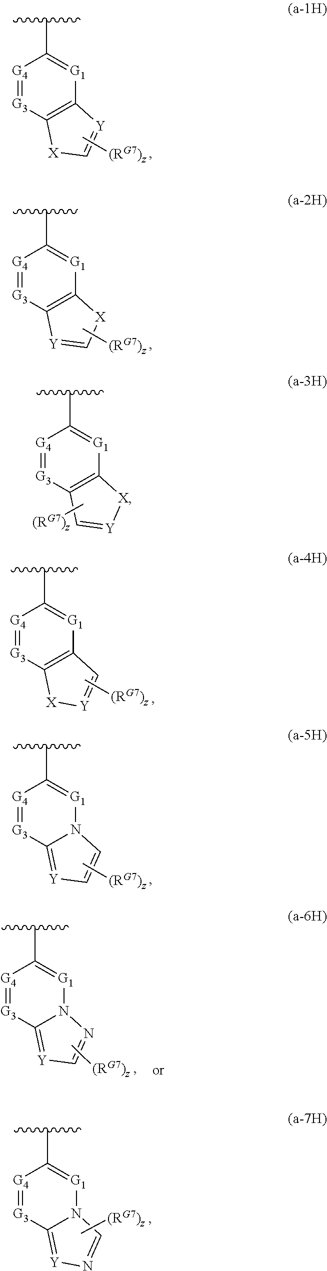

wherein:
X is O, S, NH, or NR^{G7}.
Y is N, CH, or CR^{G7}; and
z is 0 or 1;
provided if R^{G7} is a group attached to a nitrogen (N) atom, then R^{G7} is C₁₋₆ alkyl or C₁₋₆ haloalkyl.

Exemplary Embodiment 48. The compound of Exemplary Embodiment 47, or a pharmaceutically acceptable salt or tautomer thereof, wherein Ring A, when R¹ and G₂, together with the atoms to which they are attached, are joined to form a 5-membered heteroaryl ring, is a group of formula:

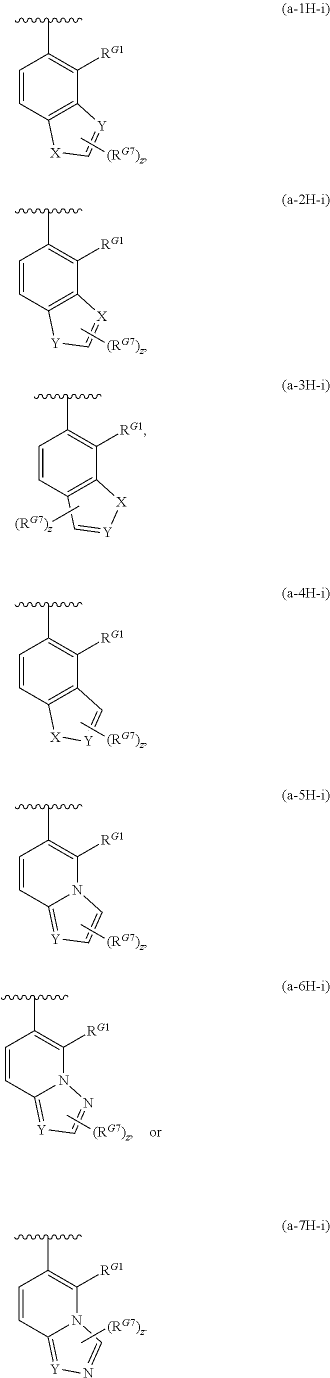

Exemplary Embodiment 49. The compound of any one of Exemplary Embodiments 1-42, or a pharmaceutically acceptable salt or tautomer thereof, wherein Ring A is a group of formula:

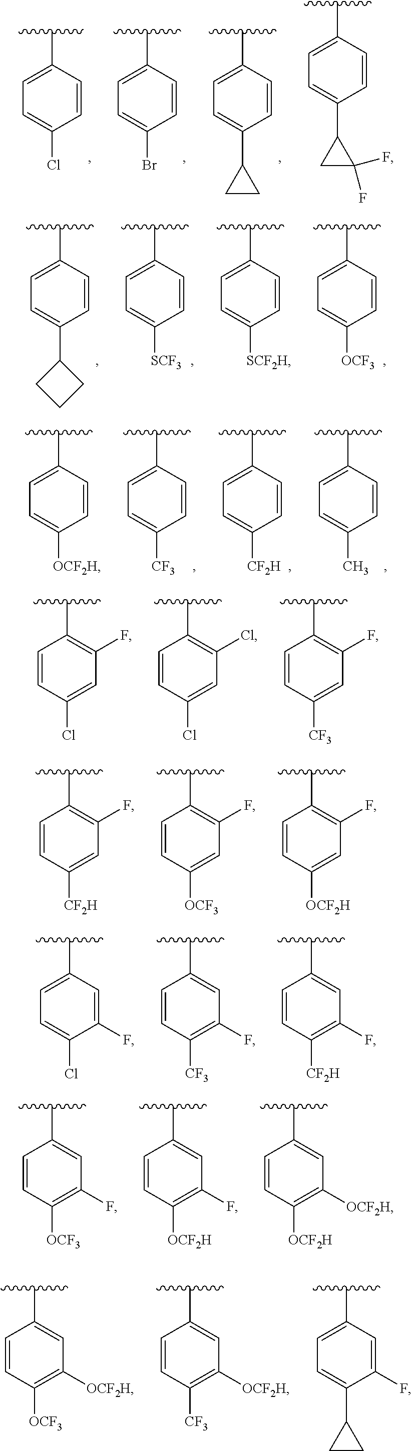

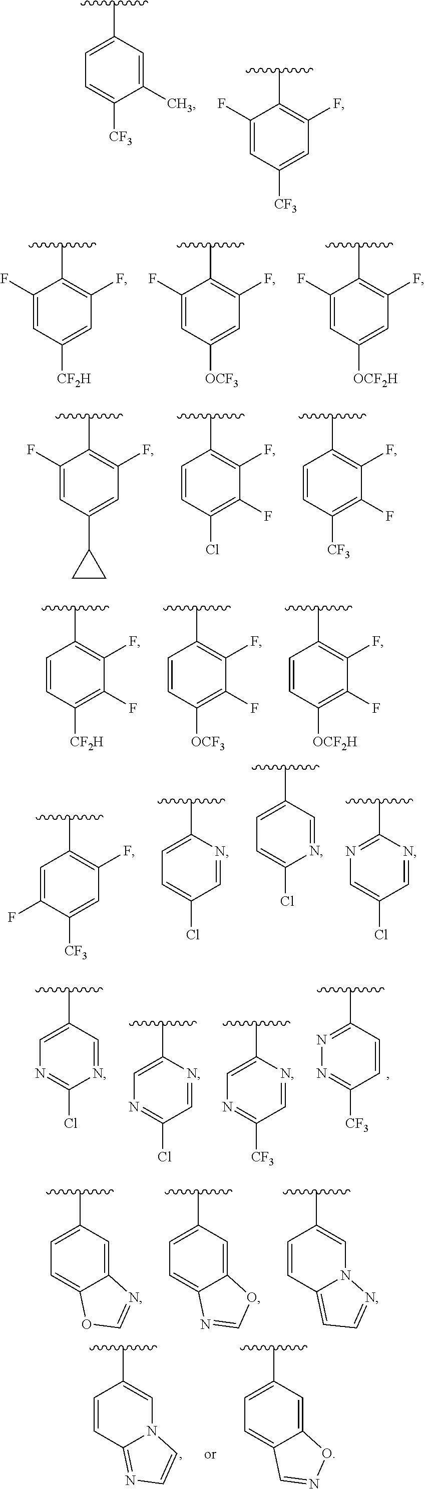

Exemplary Embodiment 50. The compound of any one of Exemplary Embodiments 1-42, or a pharmaceutically acceptable salt or tautomer thereof, wherein Ring A is a group of formula:

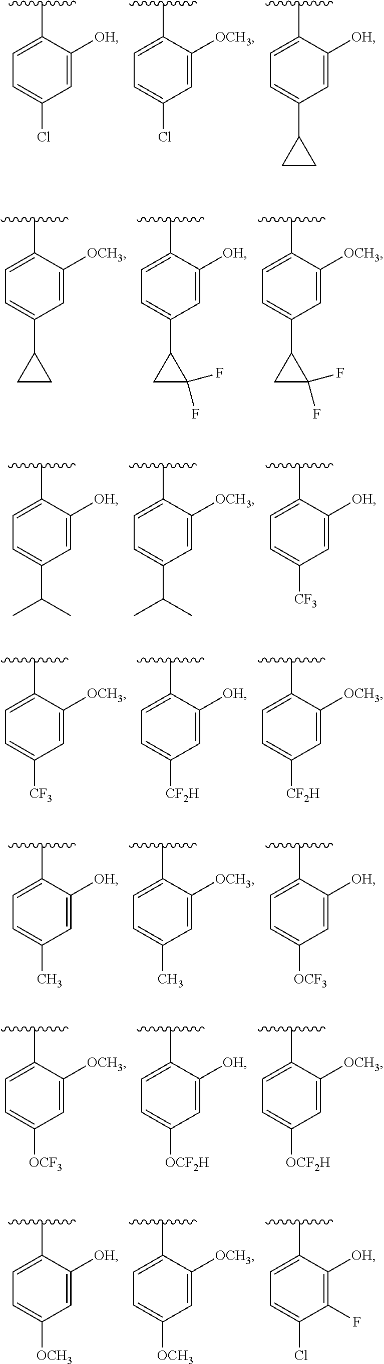

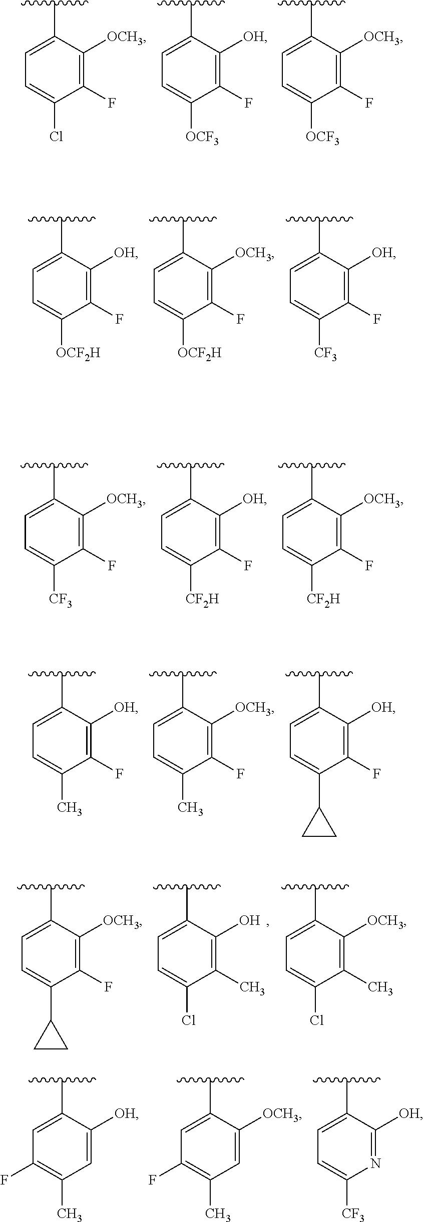

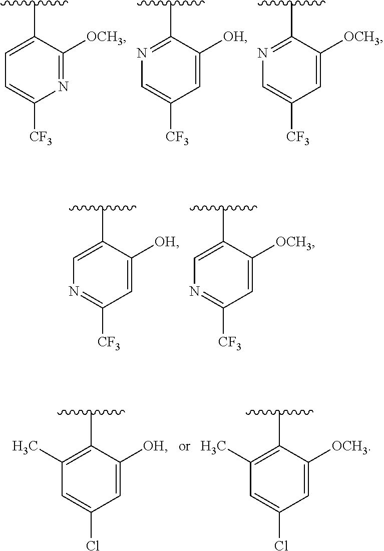

Exemplary Embodiment 51. The compound of any one of Exemplary Embodiments 1-50, or a pharmaceutically acceptable salt or tautomer thereof, wherein Ring B of formula:

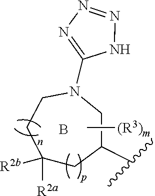

is a group of formula:

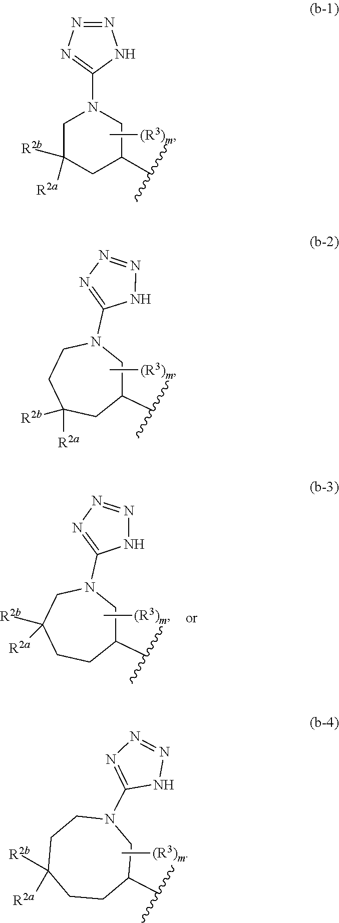

Exemplary Embodiment 52. The compound of Exemplary Embodiment 51, or a pharmaceutically acceptable salt or tautomer thereof, wherein Ring B is a group of formula:

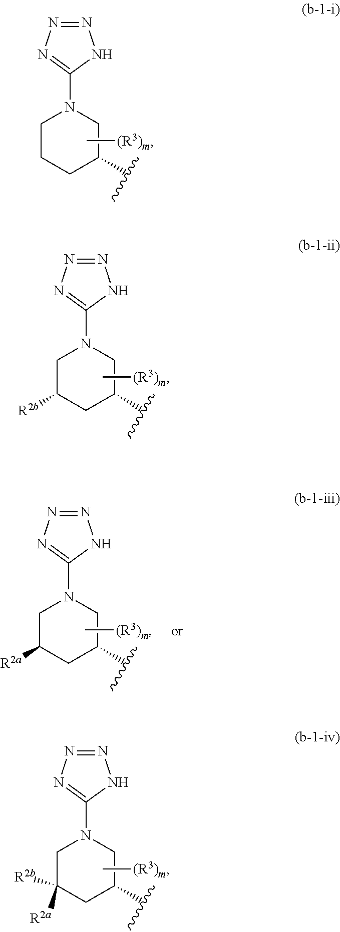

wherein each instance of R^{2a} and R^{2b} is independently halo, C₁₋₆ alkyl, C₁₋₆haloalkyl, C₃-C₄ carbocyclyl, or 3-4 membered heterocyclyl.

Exemplary Embodiment 53. The compound of Exemplary Embodiment 51, or a pharmaceutically acceptable salt or tautomer thereof, wherein Ring B of formula (b-1), (b-2), (b-3), or (b-4), when two R³ groups are joined to form a C₁₋₃ alkylene bridging group or C₁₋₃ haloalkylene bridging group, is a group of formula:

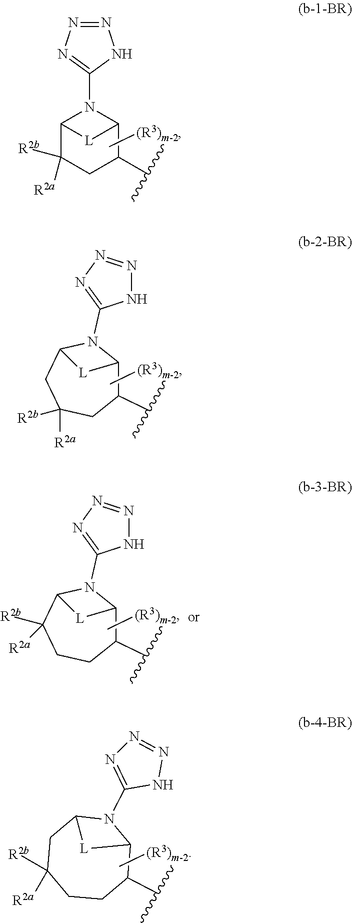

wherein L is a C₁₋₃ alkylene bridging group or C₁₋₃ haloalkylene bridging group.

Exemplary Embodiment 54. The compound of Exemplary Embodiment 53, or a pharmaceutically acceptable salt or tautomer thereof, wherein Ring B is a group of formula:

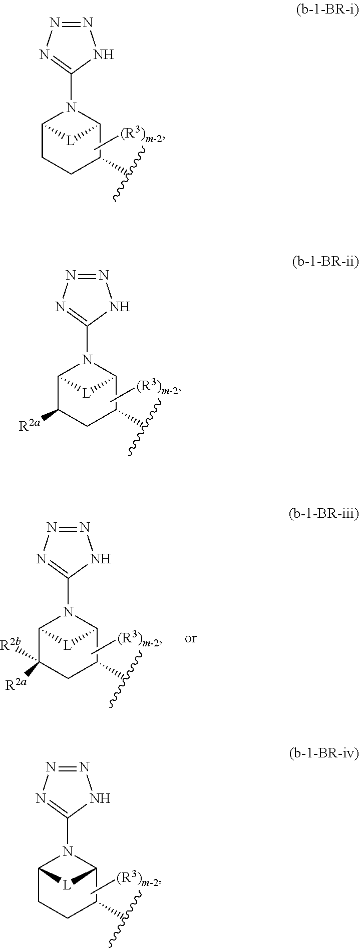

wherein each instance of R^{2a} and R^{2b} is independently halo, C₁₋₆ alkyl, C₁₋₆ haloalkyl, C₃-C₄ carbocyclyl, or 3-4 membered heterocyclyl.

Exemplary Embodiment 55. The compound of any one of Exemplary Embodiments 1-50, or a pharmaceutically acceptable salt or tautomer thereof, wherein Ring B is a group of formula:

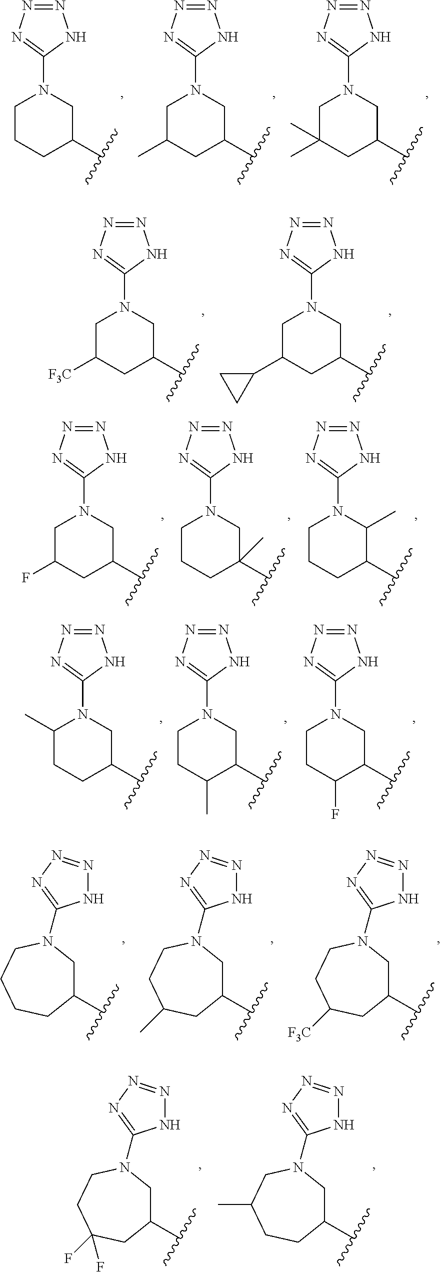

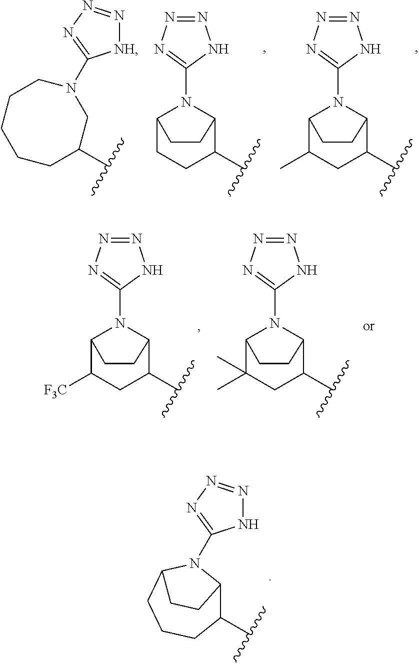

Exemplary Embodiment 56. The compound of any one of Exemplary Embodiments 1-50, or a pharmaceutically acceptable salt or tautomer thereof, wherein Ring B is a group of formula:

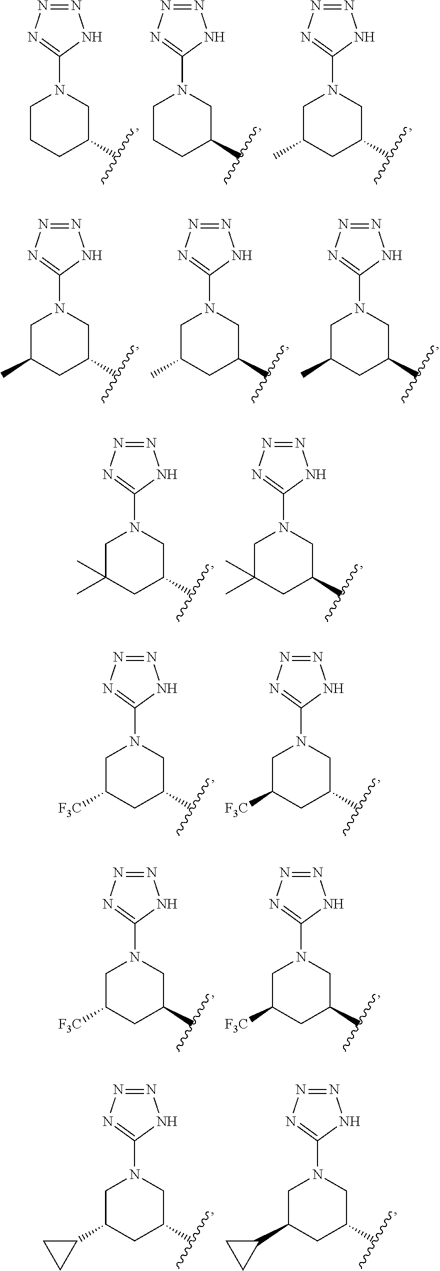

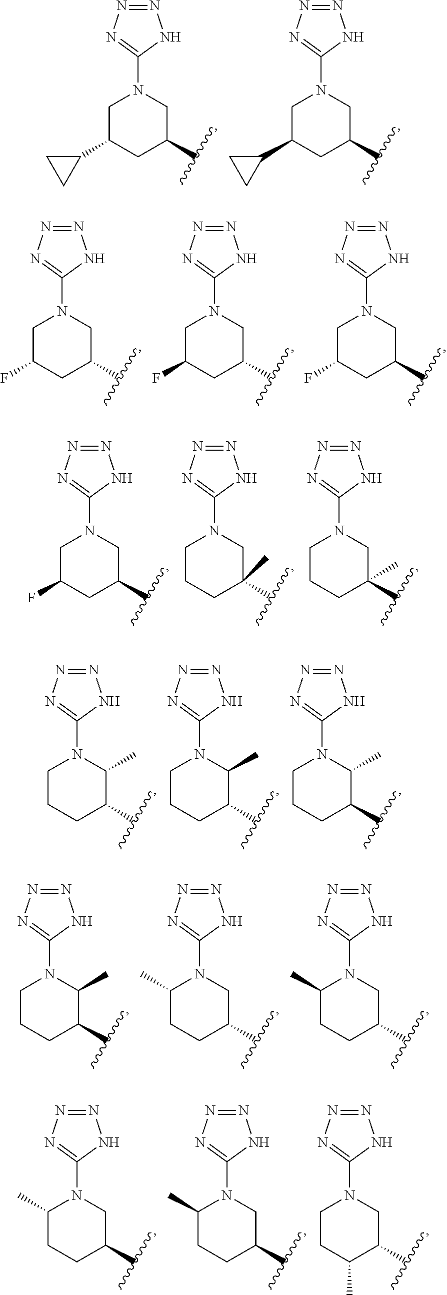

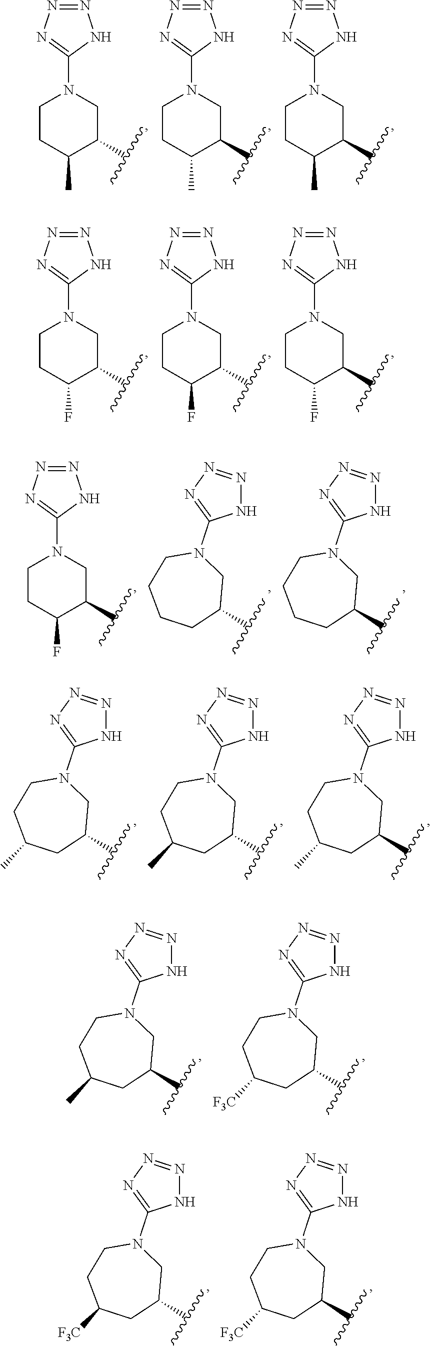

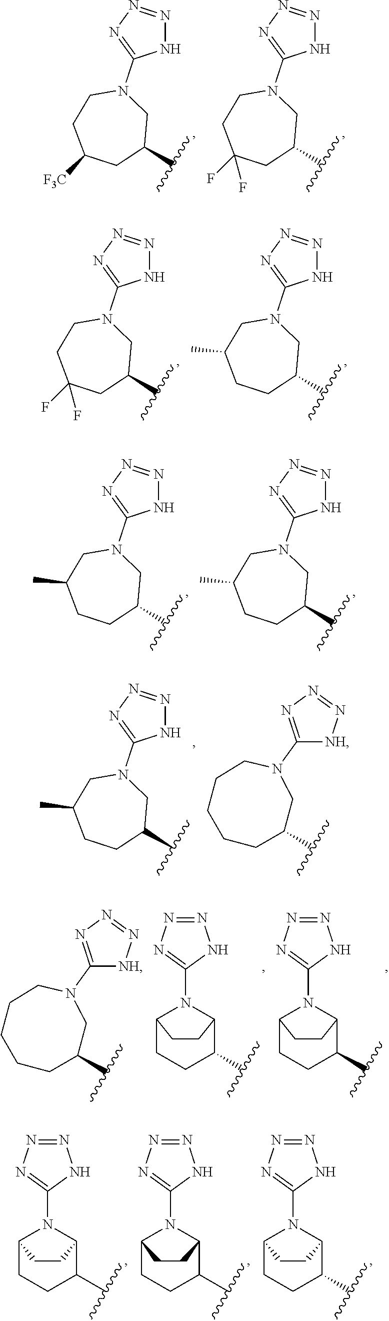

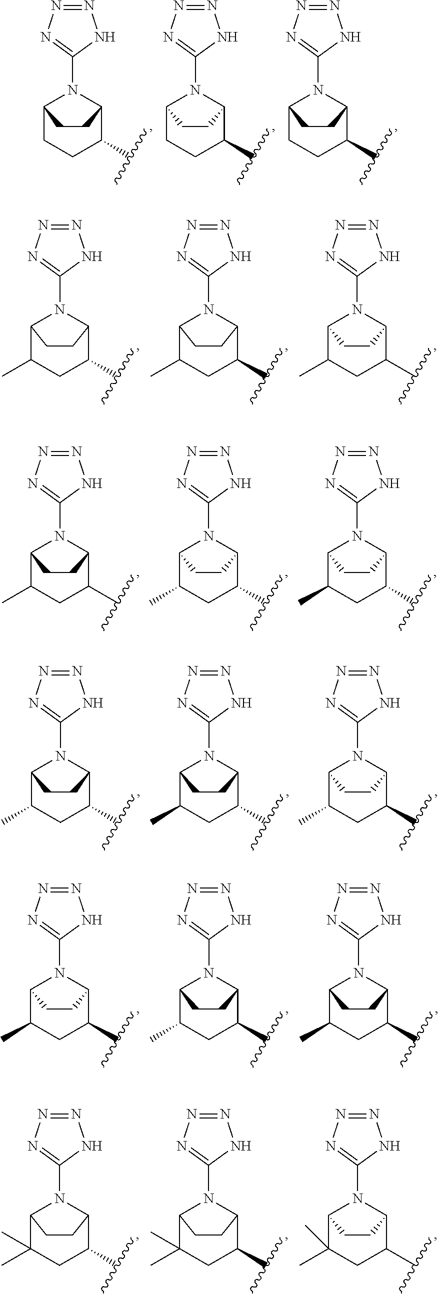

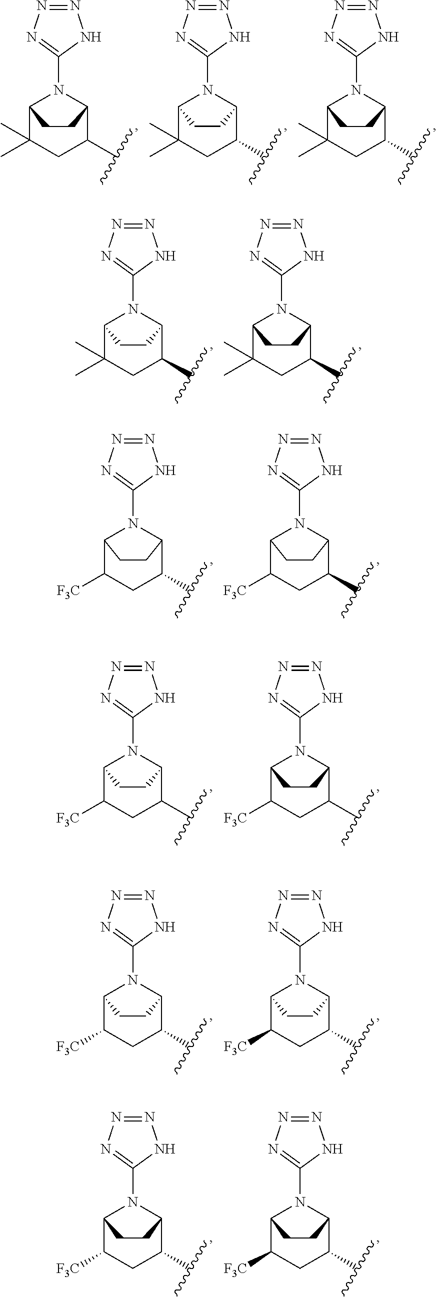

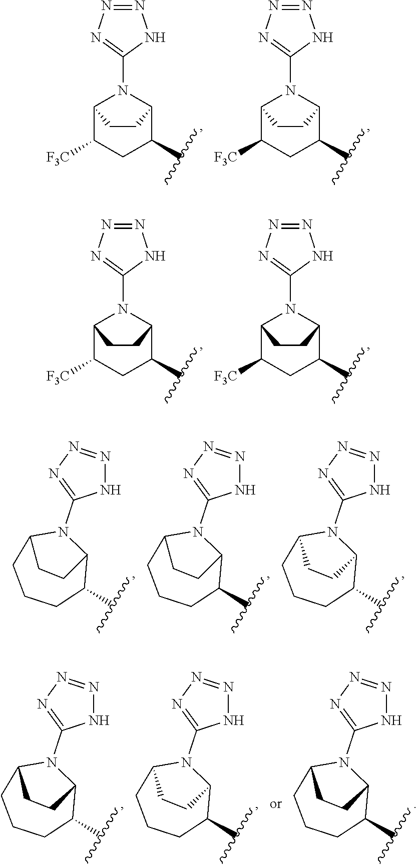

Exemplary Embodiment 57. The compound of any one of the preceding Exemplary Embodiments, wherein the compound is selected from the compounds described in Tables 1, 2, or 3, or a pharmaceutically acceptable salt or tautomer thereof.

Exemplary Embodiment 58. A pharmaceutical composition comprising the compound of any one of Exemplary Embodiments 1-57, or a pharmaceutically acceptable salt or tautomer thereof, and one or more pharmaceutically acceptable carriers.

Exemplary Embodiment 59. A method of modulating NLRP3, the method comprising administering to the subject a compound of any one of Exemplary Embodiments 1-57, or a pharmaceutically acceptable salt or tautomer thereof, or a pharmaceutical composition of Exemplary Embodiment 58.

Exemplary Embodiment 60. A method of treating or preventing a disease or disorder, the method comprising administering to the subject a compound of any one of Exemplary Embodiments 1-57, or a pharmaceutically acceptable salt or tautomer thereof, or a pharmaceutical composition of Exemplary Embodiment 58.

Exemplary Embodiment 61. The compound of any one of Exemplary Embodiments 1-57, or a pharmaceutically acceptable salt or tautomer thereof, or a pharmaceutical composition of Exemplary Embodiment 58, for use in treating or preventing a disease or disorder.

Exemplary Embodiment 62. Use of the compound of any one of Exemplary Embodiments 1-57, or a pharmaceutically acceptable salt or tautomer thereof, in the manufacture of a medicament, for the treatment or prevention of a disease or disorder.

Exemplary Embodiment 63. Use of the compound of any one of Exemplary Embodiments 1-57, or a pharmaceutically acceptable salt or tautomer thereof, for the treatment or prevention of a disease or disorder.

Exemplary Embodiment 64. The method, compound, or use of any one of Exemplary Embodiments 59-63, wherein the disease or disorder is an NLRP3-related disease or disorder.

Exemplary Embodiment 65. The method, compound, or use of any one of Exemplary Embodiments 59-64, wherein the subject is a human.

Exemplary Embodiment 66. The method, compound, or use of any one of Exemplary Embodiments 59-65, wherein the disease or disorder is a disease or disorder of the central nervous system (CNS), a disease or disorder of the peripheral nervous system (PNS), a primary neurological disease of the muscles, an inflammatory disorder, an autoimmune disorder, cancer, an infection, a metabolic disease, a cardiovascular disease, a respiratory disease, a kidney disease, a liver disease, an ocular disease, a skin disease, a lymphatic disease, a rheumatic disease, a psychological disease, graft versus host disease, pain (including disorders related to pain management), or an NLRP3-related disease in a subject that has been determined to carry a germline or somatic non-silent mutation in NLRP3.

Exemplary Embodiment 67. The method, compound, or use of Exemplary Embodiment 66, wherein the disease or disorder of the central nervous system is dementia, Alzheimer's disease (“AD”) epilepsy, traumatic brain injury (“TBI”), multiple sclerosis (“MS”), developmental disturbances, acute disseminated encephalopathy, transverse myelitis, Parkinson's disease (“PD”), amyotrophic lateral sclerosis (“ALS”), Huntington's disease (“HD”), or spinal cord injury.

Exemplary Embodiment 68. The method, compound, or use of Exemplary Embodiment 66, wherein the primary neurological disease of the muscle is a dystrophy or spinal muscular atrophy.

Exemplary Embodiment 69. The method, compound, or use of Exemplary Embodiment 66, wherein the inflammatory disorder is gout or anemia of inflammation.

Exemplary Embodiment 70. The method, compound, or use of Exemplary Embodiment 66, wherein the autoimmune disease is ulcerative colitis.

Exemplary Embodiment 71. The method, compound, or use of Exemplary Embodiment 66, wherein the cancer is skin cancer or colon cancer.

Exemplary Embodiment 72. The method, compound, or use of Exemplary Embodiment 66, wherein the infection is a neuro-infection.

Exemplary Embodiment 73. The method, compound, or use of Exemplary Embodiment 66, wherein the metabolic disease is diabetes.

Exemplary Embodiment 74. The method, compound, or use of Exemplary Embodiment 66, wherein the cardiovascular disease is stroke.

Exemplary Embodiment 75. The method, compound, or use of Exemplary Embodiment 66, wherein the respiratory disease is asthma or chronic obstructive pulmonary disease.

Exemplary Embodiment 76. The method, compound, or use of Exemplary Embodiment 66, wherein the kidney disease is acute kidney disease, a chronic kidney disease, or a rare kidney disease.

Exemplary Embodiment 77. The method, compound, or use of Exemplary Embodiment 66, wherein the liver disease is nonalcoholic fatty liver disease (NAFLD) or non-alcoholic steatohepatitis (NASH).

Exemplary Embodiment 78. The method, compound, or use of Exemplary Embodiment 66, wherein the ocular disease is optic neuritis or macular degeneration.

Exemplary Embodiment 79. The method, compound, or use of Exemplary Embodiment 66, wherein the skin disease is psoriasis, hidradenitis suppurativa (HS), or atopic dermatitis.

Exemplary Embodiment 80. The method, compound, or use of Exemplary Embodiment 66, wherein the rheumatic disease is osteoarthritis, dermatomyositis, Still's disease, or juvenile idiopathic arthritis.

Exemplary Embodiment 81. The method, compound, or use of Exemplary Embodiment 66, wherein the psychological disease is a neuropsychiatric condition selected from the group consisting of depression, major depressive disorder, and refractory depression.

Exemplary Embodiment 82. The method, compound, or use of Exemplary Embodiment 66, wherein the pain is pain management addiction, osteoarthritis pain, or allodynia.

Exemplary Embodiment 83. The method, compound, or use of Exemplary Embodiment 66, wherein the NLRP3-related disease in a subject that has been determined to carry a germline or somatic non-silent mutation in NLRP3 is cryopyrin-associated autoinflammatory syndrome.

Exemplary Embodiment 84. The method, compound, or use of Exemplary Embodiment 66, wherein the disease or disorder is dementia, Alzheimer's disease (“AD”), epilepsy, traumatic brain injury (“TBI”), multiple sclerosis (“MS”), a developmental disturbance, acute disseminated encephalopathy, transverse myelitis, Parkinson's disease (“PD”), amyotrophic lateral sclerosis (“ALS”), spinal muscular atrophy, Huntington's disease (“HD”), spinal cord injury, a dystrophy, a neuro-infection, a pain management addiction, a neuropsychiatric condition, neonatal onset multisystem inflammatory disease (“NOMID”), asthma, osteoarthritis, ulcerative colitis, gout, anemia of inflammation, Still's disease, chronic obstructive pulmonary disease (“COPD”), osteoarthritis pain, or hidradenitis suppurativa.

Exemplary Embodiment 85. A process for preparing a compound of Formula (I-A) of any one of the preceding Exemplary Embodiments, or a salt or tautomer thereof, wherein the compound is synthesized according to General Schemes A, B, or C.

### EXEMPLIFICATION

In order that this disclosure may be more fully understood, the following Examples are set forth. It should be understood that these examples are for illustrative purposes only and are not to be construed as limiting this disclosure in any manner.

### Analytical Methods

Nuclear magnetic resonance (NMR) spectra were recorded at 400 MHz as stated and at 300.3 K unless otherwise stated; the chemical shifts (δ) are reported in parts per million (ppm). Spectra were recorded using a Bruker Avance 400 instrument with 8, 16 or 32 scans. Typical NMR solvents include deuterated dimethylsulfoxide (DMSO-d6) and deuterated methanol (CD₃OD).

Gas Chromatography-Mass Spectrometry (GCMS) chromatograms and spectra were recorded using Agilent GCMS 8890-5977 and Detector Channel FID. GC Parameters: DB-5MS, 12 m×0.20 mm×0.33 um; Column Oven Temp: 50.0; Injection volume: 0.5 μL; Column Flow: 1.2 ml/min; Injection temperature: 300° C.; Injection Mode:Split; Split Ratio: 20:1; Detector temperature: 300° C.; Initial temperature: 50° C. for 1 min then 40° C./min to 300° C. for 1.75 min. Makeup Gas: He; Makeup Flow: 25.0 mL/min; H2; Flow: 30.0 mL/min; Air Flow: 400.0 mL/min; Final temperature: 300° C. The MS detector of acquisition mode: Start Time: 2.00 min; End Time: 9.00 min; Acquisition Mode: Scan; Interface Type: EI Threshold: 150; Scan Speed: 1562; Start m/z: 50.00; End m/z: 550.00; MS Source: 230.00° C.; MS Quad: 150.00° C.; Solvent Cut Time: 2.00 min.

Liquid Chromatography-Mass Spectrometry (LCMS) chromatograms and spectra were recorded using a Shimadzu LCMS-2020. Injection volumes were 0.7-8.0 μl and the flow rates were typically 0.8 or 1.2 mL/min. Detection methods were diode array (DAD) or evaporative light scattering (ELSD) as well as positive ion electrospray ionization. MS range was 100-1000 Da. Mobile phases of water and/or acetonitrile (MeCN) may contain a modifier (typically 0.01-0.04% yield) such as trifluoroacetic acid (TFA), formic acid (FA), or ammonium carbonate. ESI or ES=electrospray ionization; m/z=mass/charge; RT=retention time (minutes).

Purification/Separation Methods. The Synthetic methods describe purification and/or separation chromatographic methods which have been employed in the purification and/or isolation of the exemplified compounds. RT=retention time (minutes); Prep=Preparative High-performance liquid chromatography.

Compounds were numbered following the below numbering system, where R^{2a} and R^{2b} are not hydrogen.

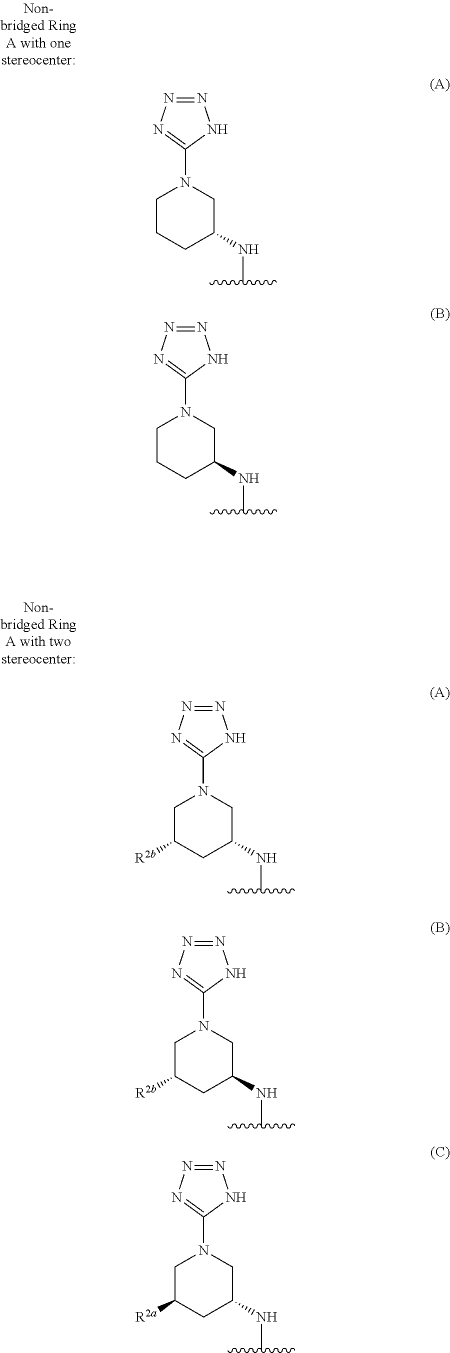

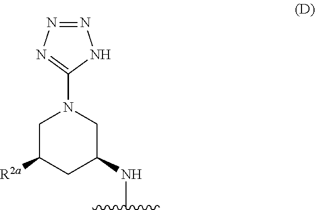

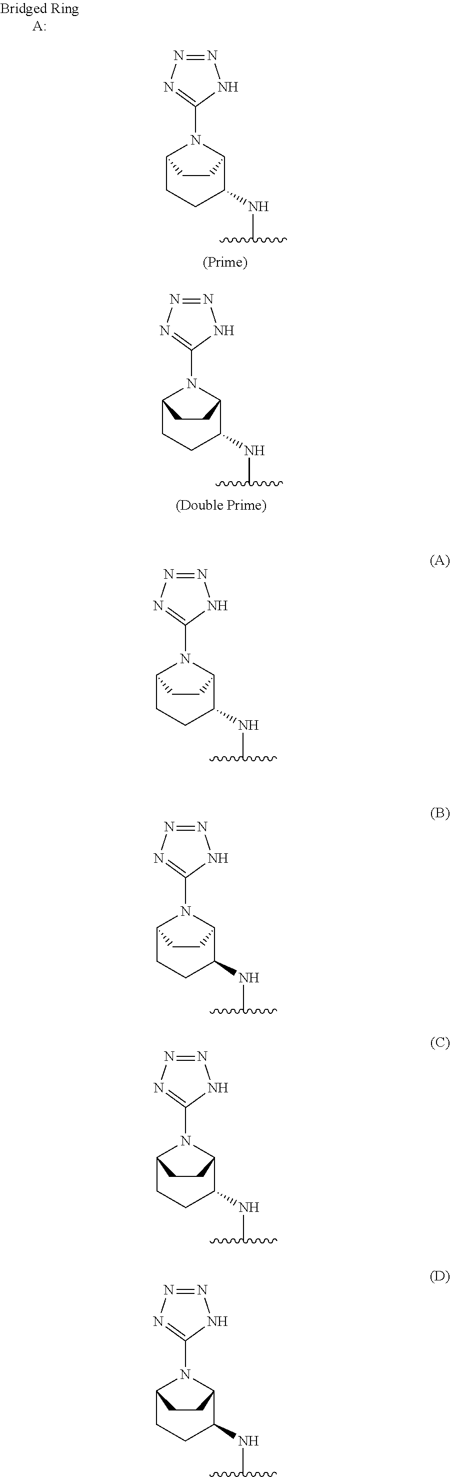 [SMILES: *N[C@@H]1CC[C@@H]2CC[C@H]1N2c1nnn[nH]1 ; *N[C@@H]1CC[C@H]2CC[C@@H]1N2c1nnn[nH]1 ; *N[C@@H]1CC[C@@H]2CC[C@H]1N2c1nnn[nH]1 ; *N[C@H]1CC[C@@H]2CC[C@H]1N2c1nnn[nH]1 ; *N[C@@H]1CC[C@H]2CC[C@@H]1N2c1nnn[nH]1 ; *N[C@H]1CC[C@H]2CC[C@@H]1N2c1nnn[nH]1]

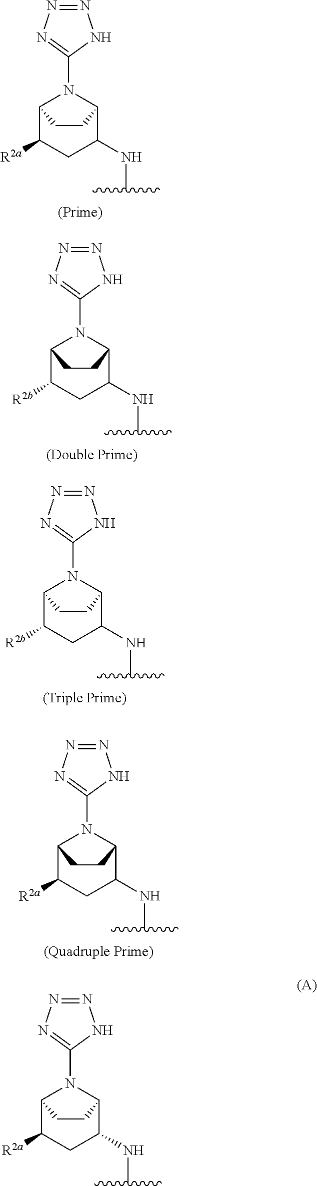

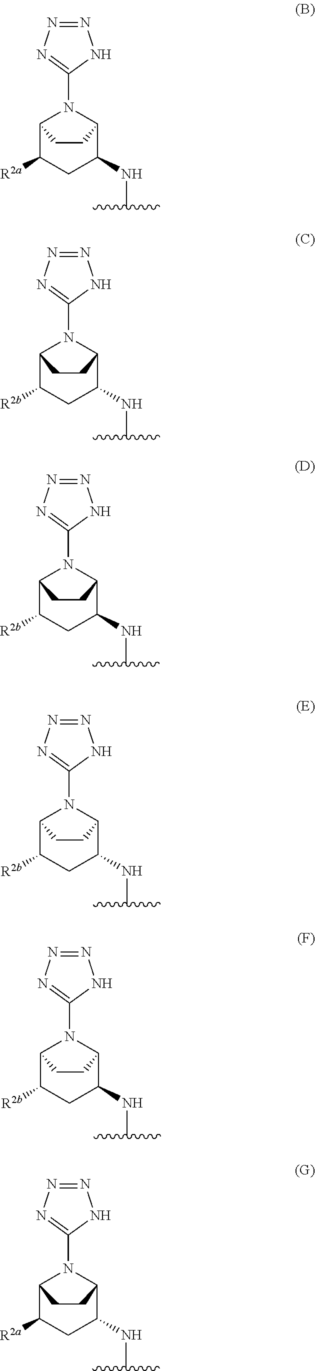

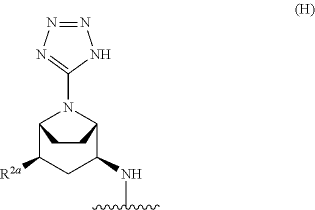

The Asterix (*) next to the Compound Number (#) signifies that arbitrary stereochemistry has been assigned. Future tense (“may be” prepared/synthesized) language signify examples to be conducted.

### SYNTHETIC EXAMPLES

### Example 1. Synthesis of 1-(4-chlorophenyl)-N-[1-(1H-1,2,3,4-tetrazol-5-yl)-5-(trifluoromethyl)azepan-3-yl]cyclopropane-1-carboxamide (Compound 1, rac-1) and Compounds 1A*, 1B*, 1C*, and 1D*

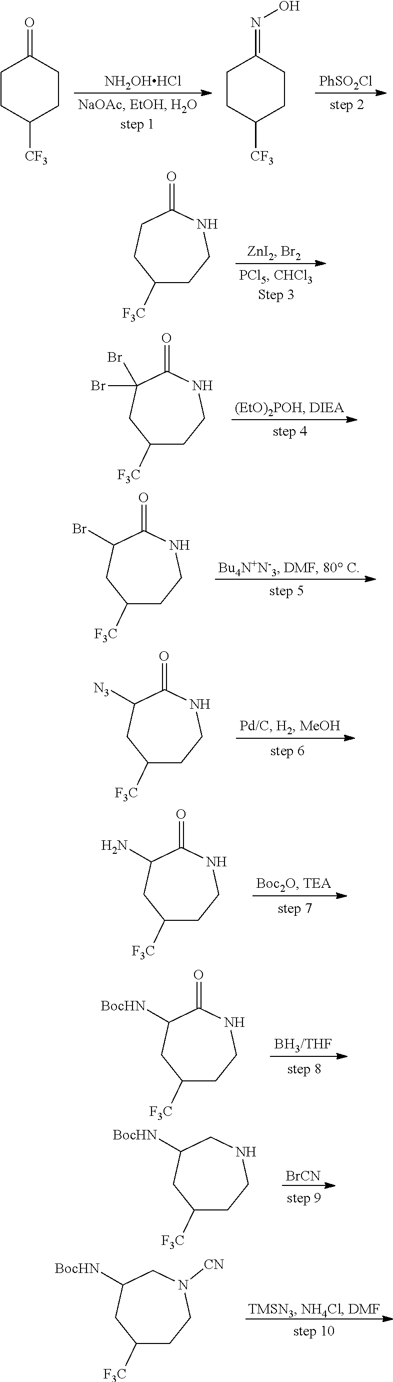

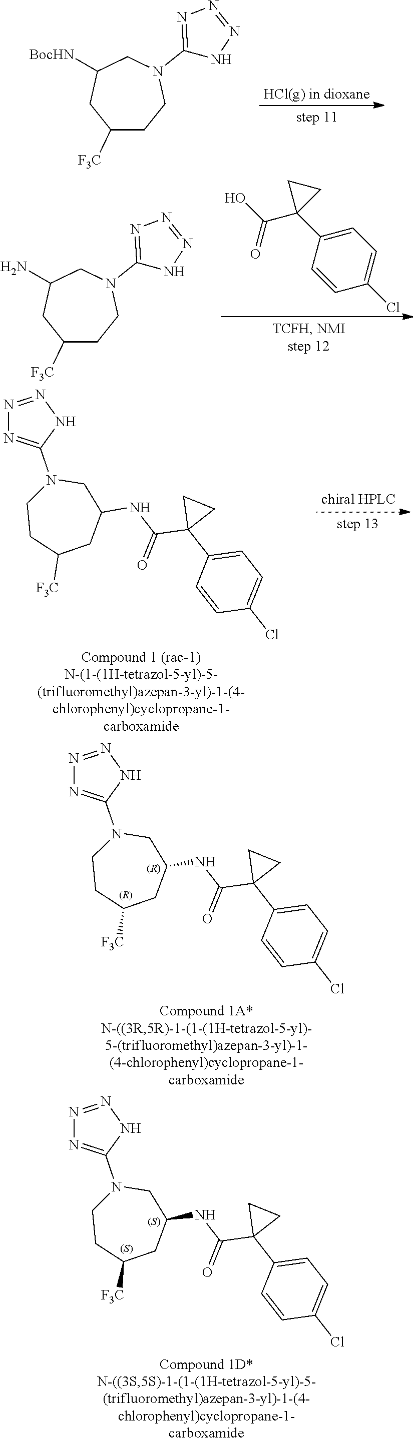

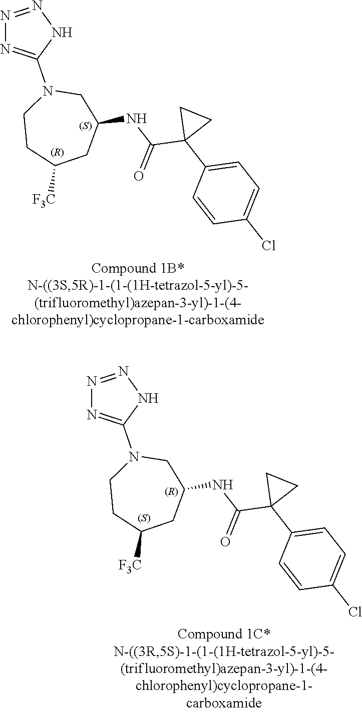

### Example 1 Follows Protocol A

Step 1: Into a 250 mL round-bottom flask was added 4-(trifluoromethyl)cyclohexan-1-one (5 g, 30.1 mmol, 1 equiv), water (50 mL), ethanol (EtOH) (70 mL), CH₃C(═O)ONa (3.70 g, 45.14 mmol, 1.5 equiv) and hydroxylamine hydrochloride (3.14 g, 45.14 mmol, 1.5 equiv) at room temperature. The resulting mixture was stirred for 3 h at 100° C. The reaction progress was monitored by LCMS. The resulting mixture was extracted with ethyl acetate (EtOAc) (2×200 mL). The combined organic layers were washed with water (lx 500 mL), dried over anhydrous Na₂SO₄, filtered, and the filtrate was concentrated under reduced pressure to provide a residue, which was purified by reverse flash chromatography (column, C18 gel; mobile phase, acetonitrile (MeCN) in water (10 mmol/L NH₄HCO₃), 0% to 100% gradient in 40 min; detector, UV 220 nm) to provide N-[4-(trifluoromethyl)cyclohexylidene]hydroxylamine (4 g, 73% yield). LCMS (ES, m/z): RT=0.681 min, m/z=182 [M+1]⁺.

Step 2: Into a 2 L round-bottom flask was added N-[4-(trifluoromethyl)cyclohexylidene]hydroxylamine (10 g, 55.2 mmol, 1 equiv), benzenesulfonyl chloride (19.5 g, 110.40 mmol, 2 equiv), Na₂CO₃ (23.4 g, 220.8 mmol, 4 equiv), propan-2-one (500 mL) and water (500 mL) at 0° C. The resulting mixture was stirred overnight at room temperature under nitrogen atmosphere. The reaction progress was monitored by LCMS. The resulting mixture was extracted with ethyl acetate (EtOAc) (2×500 mL), the combined organic layers were washed with brine (1×1 L), dried over anhydrous Na₂SO₄, filtered, and concentrated under reduced pressure to provide a residue, which was purified by reverse flash chromatography (column, C18 gel; mobile phase, Acetonitrile (MeCN) in water (0.1% NH₃. water), 0% to 100% gradient in 40 min; detector, UV 220 nm) to provide 5-(trifluoromethyl)azepan-2-one (9 g, 90% yield). LCMS (ES, m/z): RT=0.530 min, m/z=182 [M+H]⁺.

Step 3: Into a 500 mL round-bottom flask was added 5-(trifluoromethyl)azepan-2-one (8 g, 44.2 mmol, 1 equiv) and CHCl₃ (250 mL), and PCl₅ (18.4 g, 88.32 mmol, 2 equiv) at 0° C. The resulting mixture was stirred for 30 min at 0° C. under nitrogen atmosphere. To the above mixture was added ZnI₂ (0.51 g, 1.59 mmol, 0.036 equiv) at 0° C. The resulting mixture was stirred for additional 30 min at 0° C. To the above mixture was added Br₂ (14.11 g, 88.32 mmol, 2 equiv) at 0° C. The resulting mixture was stirred for additional overnight at room temperature. The reaction progress was monitored by LCMS. The reaction was quenched with sat. sodium hyposulfite (aq.500 mL.0.5 mol/L) at room temperature, extracted with Dichloromethane (DCM) (3×500 mL), and the combined organic layers were washed with water (1×1 L), dried over anhydrous Na₂SO₄, filtered, and the filtrate was concentrated under reduced pressure to provide 3,3-dibromo-5-(trifluoromethyl)azepan-2-one (6 g, 40% yield). LCMS (ES, m/z): RT=0.785 min, m/z=338 [M+H]⁺.

Step 4: Into a 250 mL vial was added 3,3-dibromo-5-(trifluoromethyl)azepan-2-one (10 g, 29.50 mmol, 1 equiv), dichloromethane (DCM) (100 mL), dichloroethylamine (diisopropylethyl amine (DIEA)) (3.81 g, 29.50 mmol, 1 equiv), and diethoxyphosphinous acid (8.15 g, 59.006 mmol, 2 equiv) at 0° C. The resulting mixture was stirred for 1 h at 0° C. under nitrogen atmosphere, then stirred for additional 1h at 50° C. under nitrogen atmosphere. The reaction progress was monitored by LCMS. The resulting mixture was extracted with dichloromethane (DCM) (2×200 mL), the combined organic layers were washed with water (1×500 mL), dried over anhydrous Na₂SO₄, filtered, concentrated under reduced pressure, and the residue was purified by reverse flash chromatography (column, silica gel; mobile phase, Acetonitrile (MeCN) in water, 0% to 100% gradient in 40 min; detector, UV 254 nm) to provide 3-bromo-5-(trifluoromethyl)azepan-2-one (5 g, 65% yield). LCMS (ES, m/z): RT=0.680 min, m/z=260 [M+1]⁺.

Step 5: Into a 250 mL round-bottom flask was added 3-bromo-5-(trifluoromethyl)azepan-2-one (7 g, 26.91 mmol, 1 equiv) and dimethylformamide (DMF) (70 mL), and tetrabutyl azide amine (11.49 g, 40.37 mmol, 1.5 equiv) at room temperature. The resulting mixture was stirred for 1 h at 100° C. under nitrogen atmosphere. The reaction progress was monitored by LCMS. The resulting mixture was extracted with ethyl acetate (EtOAc) (2×200 mL), the combined organic layers were washed with water (lx 500 mL), and dried over anhydrous Na₂SO₄. After filtration, the filtrate was concentrated under reduced pressure. The crude product, 3-azido-5-(trifluoromethyl)azepan-2-one, was used in the next step directly without further purification (4 g, 67% yield). LCMS (ES, m/z): RT=0.371 min, m/z=223 [M+1]⁺.

Step 6: Into a 500 mL round-bottom flask was added 3-azido-5-(trifluoromethyl)azepan-2-one (6 g, 27.00 mmol, 1 equiv), Pd/C (1437 mg, 13.50 mmol, 0.5 equiv), and methanol (MeOH) (200 mL) at room temperature. The resulting mixture was stirred for 1 h at room temperature under hydrogen atmosphere. The reaction progress was monitored by LCMS, and upon completion, the resulting mixture was filtered, the filter cake was washed with Methanol (MeOH) (1×50 mL). The resulting mixture, providing 3-amino-5-(trifluoromethyl)azepan-2-one, was used in the next step directly without further purification. LCMS (ES, m/z): RT=0.675 min, m/z=197 [M+1]⁺.

Step 7: Into a 500 mL round-bottom flask was added the reaction mixture from Step 6, 3-amino-5-(trifluoromethyl)azepan-2-one (25.48 mmol, 1 equiv.), ditertbutyldicarbonate (Boc₂O) (11.13 g, 50.97 mmol, 2 equiv), and triethylamine (TEA) (7.74 g, 76.46 mmol, 3 equiv) at room temperature. The resulting mixture was stirred for 1 h at room temperature under nitrogen atmosphere. The reaction progress was monitored by LCMS, and upon completion, the resulting mixture was extracted with ethyl acetate (EtOAc) (2×100 mL). The combined organic layers were washed with brine (1×100 mL), dried over anhydrous Na₂SO₄. After filtration, the filtrate was concentrated under reduced pressure. The residue was purified by silica gel column chromatography, eluting with petroleum ether(PE):ethyl acetate(EA) (1:1) to provide tert-butyl N-[2-oxo-5-(trifluoromethyl)azepan-3-yl]carbamate (3 g, 40% yield). LCMS (ES, m/z): RT=0.675 min, m/z=197 [M+1]⁺.

Step 8: Into an 8 mL vial was added tert-butyl N-[2-oxo-5-(trifluoromethyl)azepan-3-yl]carbamate (120 mg, 0.40 mmol, 1 equiv) and borane tetrahydrofuran complex (BH₃.tetrahydrofuran (THF)) (2 mL) at 0° C. The resulting mixture was stirred for 2 h at room temperature under nitrogen atmosphere. The reaction progress was monitored by LCMS, and upon completion, the reaction was quenched with methanol (MeOH) (3 mL) at 0° C. The resulting mixture was extracted with ethyl acetate (EtOAc) (3×20 mL). The combined organic layers were washed with water (1×50 mL), dried over anhydrous Na₂SO₄.

After filtration, the filtrate was concentrated under reduced pressure. This resulted in tert-butyl N-[5-(trifluoromethyl)azepan-3-yl]carbamate (80 mg, 70% yield), which was used crude in the next step. LCMS (ES, m/z): RT=0.603 min, m/z=283 [M+1]⁺.

Step 9: Into an 8 mL vial was added tert-butyl N-[5-(trifluoromethyl)azepan-3-yl]carbamate (80 mg, 0.28 mmol, 1 equiv), K₂CO₃ (117.49 mg, 0.84 mmol, 3 equiv), acetonitrile (MeCN) (2 mL), and BrCN (60.03 mg, 0.56 mmol, 2 equiv) at room temperature. The resulting mixture was stirred for 1 h at room temperature under nitrogen atmosphere. The reaction progress was monitored by LCMS. The resulting mixture was extracted with ethyl acetate (EtOAc) (3×20 mL). The combined organic layers were washed with brine (1×50 mL), dried over anhydrous Na₂SO₄. After filtration, the filtrate was concentrated under reduced pressure. This resulted in tert-butyl N-[1-cyano-5-(trifluoromethyl)azepan-3-yl]carbamate (50 mg, 57% yield). LCMS (ES, m/z): RT=0.874 min, m/z=308 [M+1]⁺.

Step 10: Into an 8 mL vial was added tert-butyl N-[1-cyano-5-(trifluoromethyl)azepan-3-yl]carbamate (60 mg, 0.19 mmol, 1 equiv), NH₄Cl (31.33 mg, 0.58 mmol, 3 equiv), azidotrimethylsilane (67.48 mg, 0.58 mmol, 3 equiv), and DMF (2 mL) at room temperature. The resulting mixture was stirred for 1 h at 120° C. under nitrogen atmosphere. The reaction progress was monitored by LCMS. The crude mixture was diluted with water (50 mL). The resulting mixture was extracted with ethyl acetate (EtOAc) (3×20 mL). The combined organic layers were washed with brine (1×50 mL), dried over anhydrous Na₂SO₄. After filtration, the filtrate was concentrated under reduced pressure. The residue was purified by reverse flash chromatography (C18 gel; mobile phase, acetonitrile (MeCN) in water (0.1% NH₃ in water), 0% to 100% gradient in 40 min; detector, UV 220 nm) to provide tert-butyl N-[1-(1H-1,2,3,4-tetrazol-5-yl)-5-(trifluoromethyl)azepan-3-yl]carbamate (40 mg, 58% yield). LCMS (ES, m/z): RT=0.751 min, m/z=351 [M+1]⁺.

Step 11: Into an 8 mL vial was added tert-butyl N-[1-(1H-1,2,3,4-tetrazol-5-yl)-5-(trifluoromethyl)azepan-3-yl]carbamate (50 mg, 0.14 mmol, 1 equiv) and HCl(gas) in 1,4-dioxane (2 mL) at room temperature. The resulting mixture was stirred for 1 h at room temperature under nitrogen atmosphere. The reaction progress was monitored by LCMS. The resulting mixture was concentrated under vacuum to provide a crude product, 1-(1H-1,2,3,4-tetrazol-5-yl)-5-(trifluoromethyl)azepan-3-amine (30 mg HCl salt) (“amine (i) reagent”), which was used in the next step directly without further purification. LCMS (ES, m/z): RT=0.184 min, m/z=251 [M+1]⁺.

Step 12: Into an 8 mL vial was added 1-(1H-1,2,3,4-tetrazol-5-yl)-5-(trifluoromethyl)azepan-3-amine (30 mg, 0.120 mmol, 1 equiv) and 1-(4-chlorophenyl)cyclopropane-1-carboxylic acid (“carboxyl (ii) reagent”) (23.57 mg, 0.12 mmol, 1 equiv), acetonitrile (MeCN) (1.5 mL), N-methyl imidazole (NMI) (39.37 mg, 0.48 mmol, 4 equiv), N,N,N′,N-tetramnethylchloroformamidinium hexafluorophosphate (TCFH) (40.37 mg, 0.14 mmol, 1.2 equiv) at room temperature. The resulting mixture was stirred for 1 h at room temperature under nitrogen atmosphere. The reaction progress was monitored by LCMS. The crude mixture was diluted with water (10 mL). The resulting mixture was extracted with ethyl acetate (EtOAc) (3×20 mL). The combined organic layers were washed with brine (1×10 mL), dried over anhydrous Na₂SO₄. After filtration, the filtrate was concentrated under reduced pressure. The residue was purified by reverse flash chromatography (C18 gel; mobile phase, acetonitrile (MeCN) in water (0.1% NH₃ in water), 0% to 100% gradient in 40 min; detector, UV 220 nm) to provide crude product, 1-(4-chlorophenyl)-N-[1-(1H-1,2,3,4-tetrazol-5-yl)-5-(trifluoromethyl)azepan-3-yl]cyclopropane-1-carboxamide (Compound 1, rac-1), (15 mg; 60% purity), which was further purified by PREP HPLC (XBridge Prep OBD C18 Column, 30*150 mm, 5 μm; mobile phase, water (10 mmol/L NH₄HCO₃) and acetonitrile (MeCN) (27% up to 37% in 8 min); Detector, UV 220 nm) to provide a mixture of trans and cis isomers (2.8 mg). LCMS (ES, m/z): RT=1.642 min, m/z=429 [M+1]⁺. ¹H NMR (400 MHz, Methanol-d₄) δ 7.45-7.33 (m, 4H), 4.19-4.04 (m, 1H), 3.82-3.63 (m, 2H), 3.56-3.42 (m, 2H), 2.28 (q, J=10.4 Hz, 1H), 2.19-2.09 (m, 1H), 2.07-1.95 (m, 1H), 1.82-1.68 (m, 1H), 1.59-1.37 (m, 3H), 1.17-1.02 (m, 2H).

Step 13: Compounds 1A*, 1B*, 1C*, and 1D* may be isolated from rac-1 by chiral HPLC.

Stereochemistry is arbitrarily assigned.

### Example 2. Synthesis of 1-(4-cyclopropylphenyl)-N-(5-methyl-1-(1H-tetrazol-5-yl)azepan-3-yl)cyclopropane-1-carboxamide (Compound 2, rac-2), and Compounds 2A*, 2B*, 2C*, and 2D*

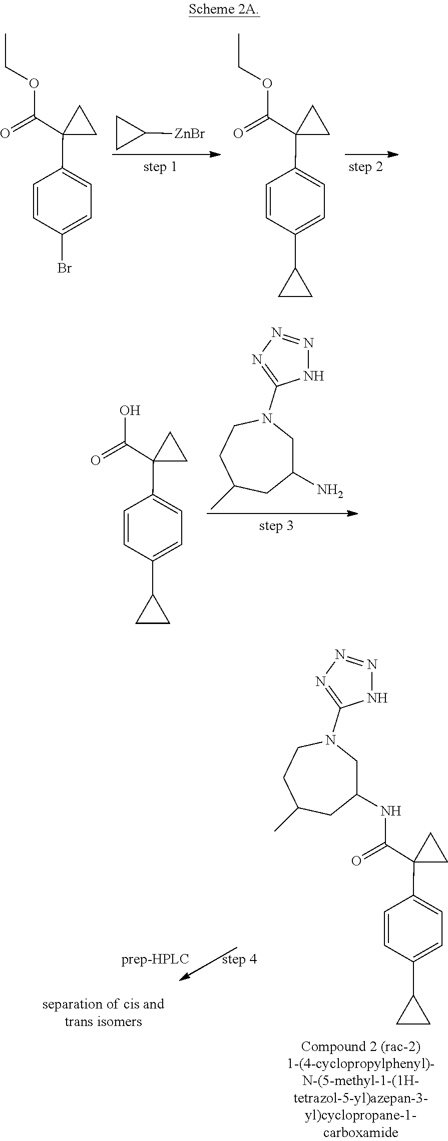

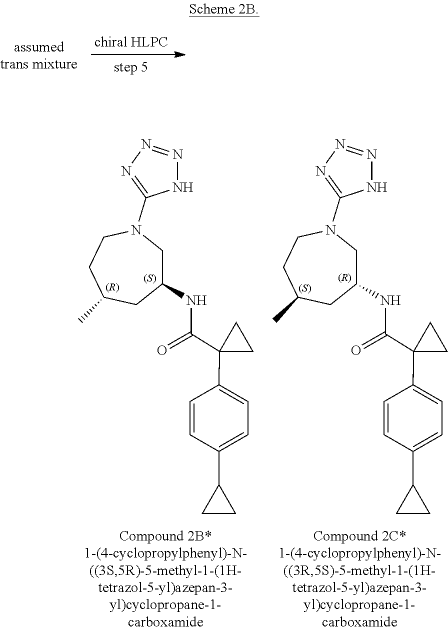

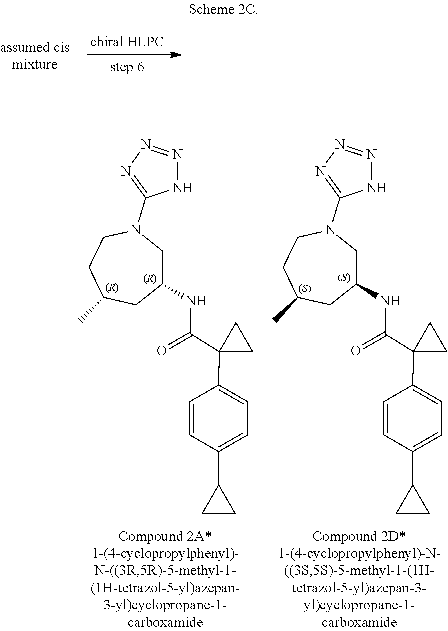

### Example 2 Follows Protocol A

Step 1: Into a 40 mL vial was added ethyl 1-(4-bromophenyl)cyclopropane-1-carboxylate (1.00 g, 3.72 mmol, 1 equiv), tetrahydrofuran (THF) (10 mL), bromo(cyclopropyl)zinc (1.73 g, 9.29 mmol, 2.50 equiv) and bis(tri-tert-butylphosphine)palladium(0) (Pd(t-Bu₃P)₂) (0.57 g, 1.12 mmol, 0.30 equiv) at room temperature under nitrogen atmosphere. The resulting mixture was stirred for 4 h at 80° C. The resulting mixture was concentrated under vacuum, and then extracted with ethyl acetate (EtOAc) (3×10 mL). The combined organic layers were washed with brine (1×10 mL) and dried over anhydrous Na₂SO₄. After filtration, the filtrate was concentrated under reduced pressure. The residue was purified by silica gel column chromatography, eluting with petroleum ether/ethyl acetate (5:1), to provide ethyl 1-(4-cyclopropylphenyl)cyclopropane-1-carboxylate (560 mg, 65% yield). LCMS (ESI): RT=1.42 min, m/z=231.0 [M+H]+.

Step 2: Into an 8 mL vial was added ethyl 1-(4-cyclopropylphenyl)cyclopropane-1-carboxylate (260 mg, 1.13 mmol, 1 equiv), ethanol (EtOH) (4 mL), water (1 mL) and NaOH (135.46 mg, 3.39 mmol, 3 equiv) at room temperature. The resulting mixture was stirred for 2 h at room temperature. The mixture was acidified to pH 3 with HCl (aq.). The resulting mixture was extracted with ethyl acetate (EtOAc) (3×10 mL). The combined organic layers were washed with brine (1×10 mL), dried over anhydrous Na₂SO₄.

After filtration, the filtrate was concentrated under reduced pressure. The residue was purified by reverse flash chromatography (C18 silica gel; mobile phase, acetonitrile (MeCN) in water, 20% to 55% gradient in 10 min; detector, UV 254 nm) to provide 1-(4-cyclopropylphenyl)cyclopropane-1-carboxylic acid (220 mg, 96% yield). LCMS (ESI): RT=0.84 min, m/z=203.0 [MH]+.

Step 3: Into an 8 mL vial was added 1-(4-cyclopropylphenyl)cyclopropane-1-carboxylic acid (“carboxyl (ii) reagent”) (123.67 mg, 0.61 mmol, 1 equiv), dimethyl formamide (DMF) (2 mL), 5-methyl-1-(1H-1,2,3,4-tetrazol-5-yl)azepan-3-amine (“amine (i) reagent”) (120 mg, 0.61 mmol, 1 equiv), (1-[bis(dimethylamino)methylene]-1H-1,2,3-triazolo[4,5-b]pyridinium 3-oxide hexafluorophosphate (HATU) (302.24 mg, 0.79 mmol, 1.3 equiv) and triethylamine (TEA) (185.62 mg, 1.83 mmol, 3 equiv) at room temperature. The resulting mixture was stirred for 4 h at room temperature then extracted with ethyl acetate (EtOAc) (3×5 mL). The combined organic layers were washed with brine (1×5 mL), dried over anhydrous Na₂SO₄. After filtration, the filtrate was concentrated under reduced pressure to provide crude 1-(4-cyclopropylphenyl)-N-(5-methyl-1-(1H-tetrazol-5-yl)azepan-3-yl)cyclopropane-1-carboxamide (Compound 2, rac-2) (70 mg, 85% purity).

Step 4: The residue was purified by PREP HPLC (Xselect CSH C18 OBD Column 30*150 mm 5 um; mobile phase, acetonitrile (MeCN) and water (0.05% trifluoroacetic acid (TFA)) (37% water, 0.05% trifluoroacetic acid (TFA), up to 47% in 8 min, up to 54% in 4 min); Detector, UV 254 nm) to provide an assumed mixture of trans isomers (RT (min)=7.82, 40 mg) and assumed mixture of cis isomers (RT (min)=14.35, 40 mg), stereochemistry arbitrarily assigned.

Step 5: The assumed trans mixture product (40 mg) was purified by Chiral-PREP HPLC (Lux 5 um Cellulose-4, 2.12*25 cm, 5 μm; Mobile Phase A: Hexanes (0.1% trifluoroacetic acid (TFA), Mobile Phase B: isopropanol; Flow rate: 20 mL/min; Gradient: 50% B to 50% B in 17 min; Wave Length: 220/254 nm) to provide Compound 2B* (RT(min): 9.58, 2.8 mg) and Compound 2C* (RT(min): 14.35, 2.8 mg). Stereochemistry was arbitrarily assigned.

Compound 2B*: LCMS (ESI): RT=0.84 min, m/z=381.0 [M+H]+; 1H NMR (400 MHz, Methanol-d₄) δ 7.10-7.03 (m, 2H), 6.95-6.88 (m, 2H), 4.19 (d, J=5.0 Hz, 1H), 3.85-3.76 (m, 1H), 3.54 (dd, J=15.0, 1.7 Hz, 1H), 3.45-3.35 (m, 1H), 3.12-3.00 (m, 1H), 1.93-1.82 (m, 1H), 1.81-1.57 (m, 3H), 1.56-1.28 (m, 5H), 1.07-1.00 (m, 2H), 1.00-0.93 (m, 5H), 0.76-0.62 (m, 2H).

Compound 2C*: LCMS (ESI): RT=0.83 min, m/z=381.0 [M+H]+; 1H NMR (400 MHz, Methanol-d₄) δ 7.09-7.03 (m, 2H), 6.91 (d, J=7.9 Hz, 2H), 4.19 (d, J=5.0 Hz, 1H), 3.85-3.76 (m, 1H), 3.58-3.50 (m, 1H), 3.45-3.35 (m, 1H), 3.12-3.00 (m, 1H), 1.93-1.82 (m, 1H), 1.80-1.57 (m, 3H), 1.55-1.25 (m, 5H), 1.07-1.01 (m, 2H), 1.00-0.92 (m, 5H), 0.76-0.61 (m, 2H).

Step 6: The assumed cis mixture product (40 mg) was further purified by Chiral-PREP HPLC (Lux 5 um Cellulose-4, 2.12*25 cm, 5 um; mobile phase, hexanes (0.1% trifluoroacetic acid (TFA)) and methanol (MeOH):ethanol (EtOH) (1:1) (hold 50% MeOH:EtOH=1:1 in 8 min); Detector, UV 254 nm) to provide Compound 2D* (RT (min)=6.59, 8.9 mg) and Compound 2A* (RT (min)=7.56, 17.5 mg).

Stereochemistry was arbitrarily assigned.

Compound 2A*: LCMS (ESI): RT=1.63 min, m/z=381.0 [M+H]⁺. ¹H NMR (400 MHz, Methanol-d₄) δ 7.32-7.25 (m, 2H), 7.14-7.07 (m, 2H), 4.01-3.90 (m, 1H), 3.64-3.53 (m, 2H), 3.53-3.40 (m, 2H), 1.99-1.88 (m, 1H), 1.88-1.80 (m, 1H), 1.71-1.60 (m, 2H), 1.58-1.40 (m, 3H), 1.31 (s, OH), 1.28-1.16 (m, 1H), 1.16-1.03 (m, 2H), 1.03-0.88 (m, 5H), 0.76-0.67 (m, 2H).

Compound 2D*: LCMS (ESI): RT=0.94 min, m/z=381.0 [M+H]+; ¹H NMR (400 MHz, Methanol-d₄) δ 7.31-7.25 (m, 2H), 7.14-7.07 (m, 2H), 4.01-3.90 (m, 1H), 3.69-3.53 (m, 2H), 3.53-3.39 (m, 3H), 1.99-1.88 (m, 1H), 1.85 (d, J=14.5 Hz, 1H), 1.67 (d, J=13.9 Hz, 2H), 1.58-1.48 (m, 3H), 1.28-1.15 (m, 1H), 1.13-1.03 (m, 2H), 1.03-0.93 (m, 5H), 0.76-0.67 (m, 2H).

### Example 3. Synthesis of 1-(4-(difluoromethoxy)-2-fluorophenyl)-N-(5-methyl-1-(1H-tetrazol-5-yl)azepan-3-yl)cyclopropane-1-carboxamide (Compound 3, rac-3) and Compounds 3A*, 3B*, 3C*, and 3D*

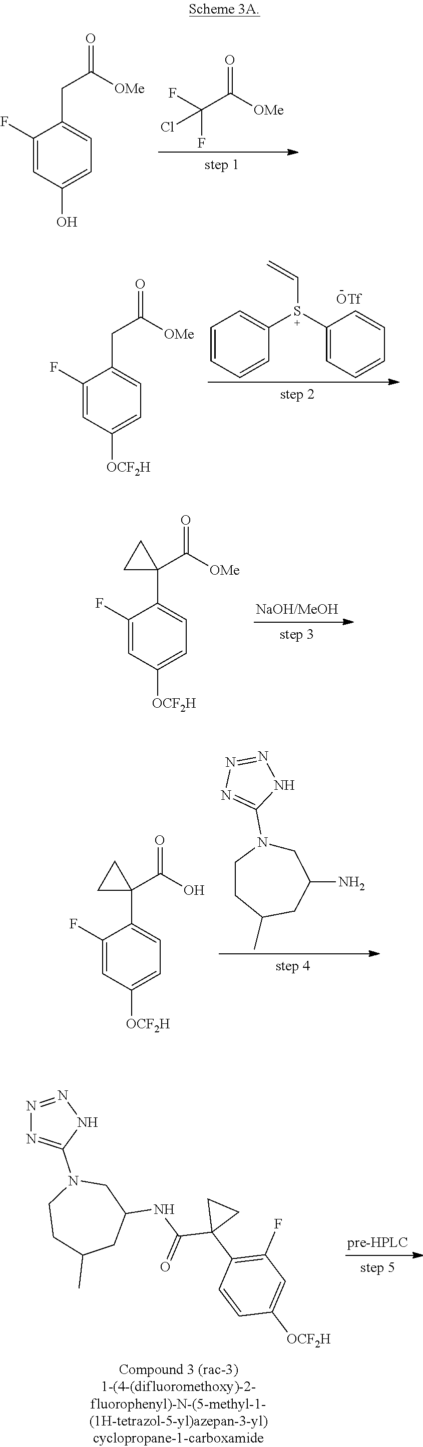

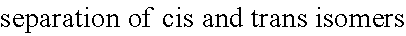

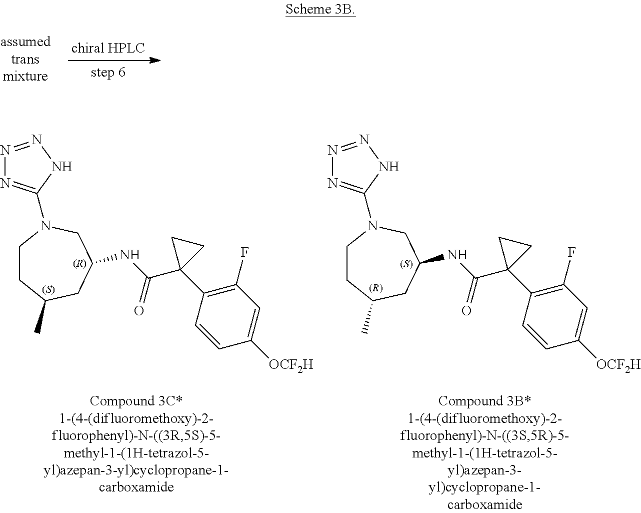

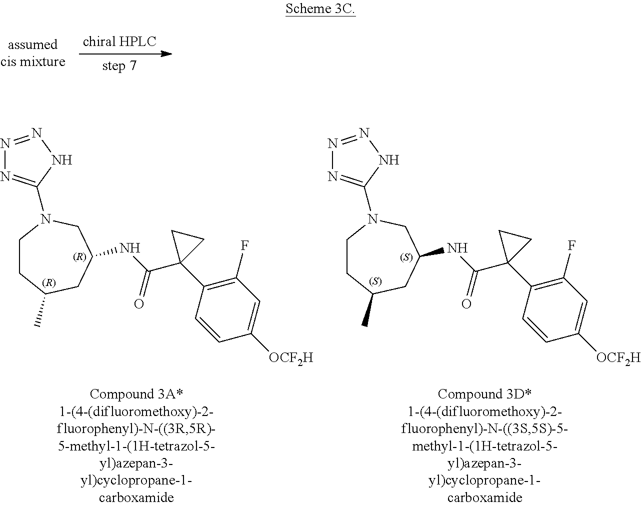

### Example 3 Follows Protocol A

Step 1: Into an 8 mL vial was added methyl 2-(2-fluoro-4-hydroxyphenyl)acetate (450 mg, 2.443 mmol, 1 equiv), sodium 2-chloro-2,2-difluoroacetate (558.79 mg, 3.665 mmol, 1.5 equiv), K₂CO₃ (1013.09 mg, 7.329 mmol, 3 equiv) and dimethylformamide (DMF) (3 mL). The resulting mixture was stirred for 2 h at 80° C. under air atmosphere. Desired product could be detected by LCMS. The aqueous layer was extracted with ethyl acetate (EtOAc) (3×15 mL). The residue was purified by silica gel column chromatography, eluting with petroleum ether:ethyl acetate (6:1), to provide methyl 2-[4-(difluoromethoxy)-2-fluorophenyl]acetate (300 mg, 52% yield).

Step 2: Into a 20 mL vial was added methyl 2-[4-(difluoromethoxy)-2-fluorophenyl]acetate (320 mg, 1.367 mmol, 1 equiv), diphenylbinylsulfonium triflate (594.23 mg, 1.640 mmol, 1.2 equiv), 1,8-diazabicyclo[5.4.0]undec-7-ene (DBU) (624.11 mg, 4.101 mmol, 3 equiv) and dimethyl sulfoxide (DMSO) (5 mL, 4.224 mmol). The resulting mixture was stirred for 1 h at room temperature under air atmosphere. Desired product could be detected by LCMS. The aqueous layer was extracted with ethyl acetate (EtOAc) (3×10 mL). The residue was purified by silica gel column chromatography, eluting with dichloromethane (DCM):petroleum ether (1:4), to provide 1-[4-(difluoromethoxy)-2-fluorophenyl]cyclopropane-1-carboxylic acid (305 mg, 86% yield).

Step 3: Into a 20 mL vial was added 1-[4-(difluoromethoxy)-2-fluorophenyl]cyclopropane-1-carboxylic acid (300 mg, 1.153 mmol, 1 equiv), NaOH (138.34 mg, 3.459 mmol, 3 equiv), methanol (MeOH) (5 mL) and water (1 mL). The resulting mixture was stirred overnight at 40° C. under air atmosphere. Desired product could be detected by LCMS. The resulting residue was dried under vacuum. The mixture was acidified to pH 3 with conc. HCl. The aqueous layer was extracted with ethyl acetate (EtOAc) (3×10 mL) to provide 1-[4-(difluoromethoxy)-2-fluorophenyl]cyclopropane-1-carboxylic acid (284 mg, 85% yield).

Steps 4-5: Into an 8 mL vial was added 1-[4-(difluoromethoxy)-2-fluorophenyl]cyclopropane-1-carboxylic acid (“carboxyl (ii) reagent”) (156.80 mg, 0.637 mmol, 1 equiv), (1-[bis(dimethylamino)methylene]1H-1,2,3-triazolo[4,5-b]pyridinium 3-oxide hexafluorophosphate (HATU) (242.18 mg, 0.637 mmol, 1 equiv), triethylamine (TEA) (193.36 mg, 1.911 mmol, 3 equiv) and dimethyl formamide (DMF) (2.5 mL) for 30 min at room temperature. To the above mixture was added 5-methyl-1-(1H-1,2,3,4-tetrazol-5-yl)azepan-3-amine (“amine (i) reagent”) (150 mg, 0.764 mmol, 1.2 equiv). The resulting mixture was stirred for additional 2 h at room temperature. Desired product could be detected by LCMS. The residue was purified by reverse flash chromatography (C18 silica gel; mobile phase, acetonitrile (MeCN): water=3:7) to provide 1-(4-(difluoromethoxy)-2-fluorophenyl)-N-(5-methyl-1-(1H-tetrazol-5-yl)azepan-3-yl)cyclopropane-1-carboxamide (Compound 3, rac-3) as a mixture of cis and trans isomers. The crude product was purified by PREP HPLC (XBridge Shield RP18 OBD Column, 30*150 mm, 5 μm; Mobile Phase A: water (10 mmol/L NH₄HCO₃), Mobile Phase B: acetonitrile (MeCN); Flow rate: 60 mL/min; Gradient: 20% B to 35% B in 8 min, 35% B; Wave Length: 254 nm; RT(min): 7.5 min. for the trans isomer and RT(min): 9.8 min. for the cis isomer) to provide a mixture of assumed cis isomers (12 mg, 4.4% yield) as the second eluting peak, and a mixture of assumed trans isomers (22 mg, 8.1% yield) as the first eluting peak, stereochemistry arbitrarily assigned.

Step 6: The assumed mixture of trans isomers from Step 5 was purified by PREP-CHIRAL HPLC (Lux 5 um Cellulose-4, 2.12*25 cm, 5 m; Mobile Phase A: Hexanes (0.1% trifluoroacetic acid (TFA)), Mobile Phase B: isopropyl alcohol (IPA); Flow rate: 20 mL/min; Gradient: 30% B to 30% B in 13 min; Wave Length: 220254 nm) to provide Compound 3B* (RT(min): 8.71, 5.20 mg, 2% yield) and Compound 3C* (RT(min): 11.29, 4.9 mg, 2% yield). Stereochemistry was arbitrarily assigned.

Compound 3B*: LCMS (ES, m/z): RT=0.99 min, m/z=425.2[M+H]⁺; ¹H NMR (400 MHz, Methanol-d4) δ 7.33-7.26 (m, 1H), 7.14-6.73 (m, 3H), 4.22-4.13 (m, 1H), 3.83-3.74 (m, 1H), 3.61-3.54 (m, 1H), 3.49-3.41 (m, 1H), 3.26-3.17 (m, 1H), 1.86-1.77 (m, 2H), 1.69-1.50 (m, 4H), 1.47-1.38 (m, 1H), 1.18-1.10 (m, 1H), 1.04-0.95 (m, 4H).

Compound 3C*: LCMS (ES, m/z): RT=0.99 min, m/z=425.2[M+H]⁺; ¹H NMR (400 MHz, Methanol-d4) δ 7.33-7.26 (m, 1H), 7.14-6.73 (m, 3H), 4.22-4.13 (m, 1H), 3.83-3.74 (m, 1H), 3.61-3.54 (m, 1H), 3.49-3.41 (m, 1H), 3.26-3.17 (m, 1H), 1.86-1.77 (m, 2H), 1.69-1.50 (m, 4H), 1.47-1.38 (m, 1H), 1.18-1.10 (m, 1H), 1.04-0.95 (m, 4H).

Step 7: The assumed mixture of cis isomers from Step 5 was purified by Chiral HPLC (Lux 5 um Cellulose-4, 2.12*25 cm, 5 m; Mobile Phase A: hexanes (0.1% trifluoroacetic acid (TFA)), Mobile Phase B: isopropanol (IPA); Flow rate: 20 mL/min; Gradient: 30% B to 30% B in 11 min; Wave Length: 220254 nm) to provide Compound 3A* (RT(min): 9.57, 2.0 mg) and Compound 3D* (RT(min): 7.64, 2.0 mg). Stereochemistry was arbitrarily assigned.

Compound 3A*: LCMS (ES, m/z): RT=1.11 min, m/z=425.2[M+H]+; ¹H NMR (400 MHz, Methanol-d4) δ 7.48-7.41 (m, 1H), 7.19-6.78 (m, 3H), 4.13-3.95 (m, 1H), 3.68-3.59 (m, 2H), 3.59-3.49 (m, 1H), 3.43-3.35 (m, 1H), 1.84-1.75 (m, 1H), 1.75-1.68 (m, 1H), 1.67-1.60 (m, 1H), 1.61-1.55 (m, 2H), 1.49-1.36 (m, 1H), 1.17-1.03 (m, 3H), 0.97 (d, J=6.8 Hz, 3H).

Compound 3D*: LCMS (ES, m/z): RT=1.11 min, m/z=425.2[M+H]+; ¹H NMR (400 MHz, Methanol-d4) δ 7.48-7.41 (m, 1H), 7.18-6.78 (m, 3H), 4.13-3.99 (m, 1H), 3.67-3.57 (m, 2H), 3.57-3.49 (m, 1H), 3.43-3.35 (m, 1H), 1.84-1.75 (m, 1H), 1.74-1.68 (m, 1H), 1.68-1.61 (m, 1H), 1.61-1.54 (m, 2H), 1.49-1.37 (m, 1H), 1.17-1.02 (m, 3H), 0.99-0.91 (m, 3H).

### Example 4. Synthesis of 1-(4-chloro-2,3-difluorophenyl)-N-(5-methyl-1-(1H-tetrazol-5-yl)azepan-3-yl)cyclopropane-1-carboxamide (Compound 4, rac-4) and Compounds 4A*, 4B*, 4C*, and 4D*

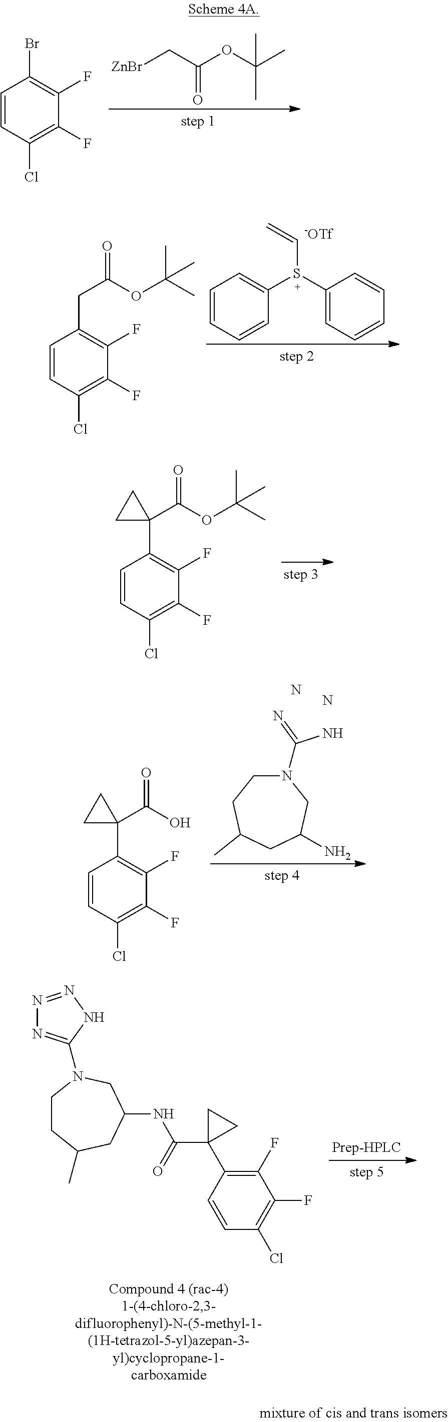

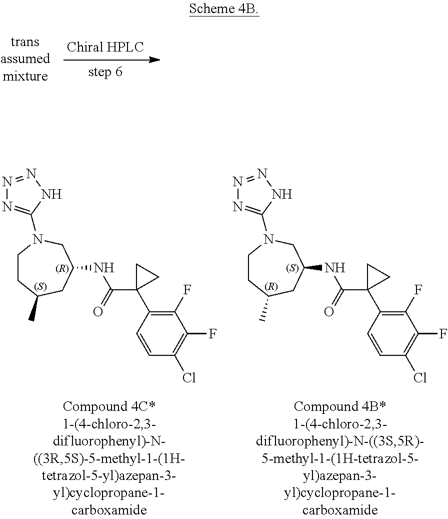

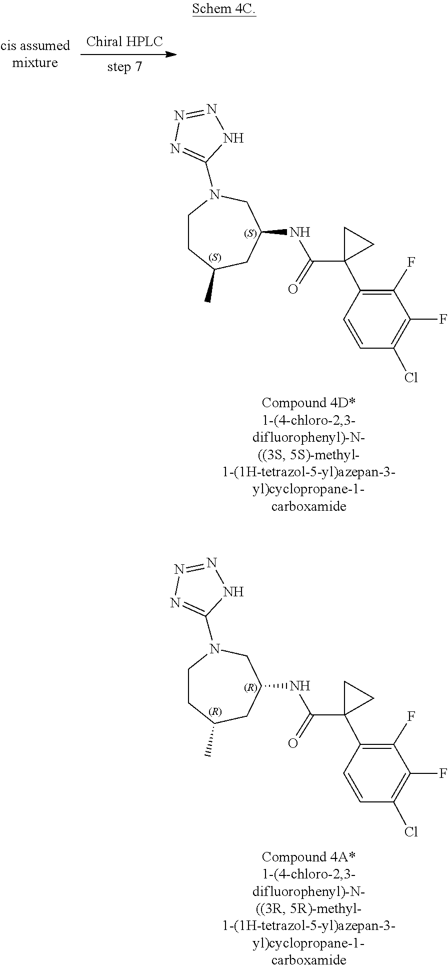

### Example 4Follows Protocol A

Step 1: Into a 40 mL round-bottom flask was added 1-bromo-4-chloro-2,3-difluorobenzene (100 mg, 0.44 mmol, 1 equiv), bis(tri-tert-butylphosphine)palladium(0) (Pd(t-Bu₃P)₂) (22.47 mg, 0.04 mmol, 0.1 equiv), tetrahydrofuran (THF) (3 mL) and tert-butyl 2-(bromozincio)acetate (229.03 mg, 0.88 mmol, 2 equiv) at room temperature. The mixture was stirred for 2 h at 80° C. under nitrogen atmosphere. Desired product could be detected by GCMS. The residue was purified by silica gel column chromatography, eluting with petroleum ether:ethyl acetate (8:1) to provide tert-butyl 2-(4-chloro-2,3-difluorophenyl)acetate (30 mg, 26% yield).

Step 2: Into a 25 mL round-bottom flask was added tert-butyl 2-(4-chloro-2,3-difluorophenyl)acetate (180 mg, 0.68 mmol, 1 equiv) and ethenyldiphenylsulfanium (292.35 mg, 1.37 mmol, 2 equiv, triflate), 1,8-diazabicyclo[5.4.0]undec-7-ene (DBU) (312.97 mg, 2.05 mmol, 3 equiv), and dimethylsulfoxide (DMSO) (3 mL) at room temperature. The resulting mixture was stirred for 6 h at room temperature. The residue was purified by Prep-TLC (petroleum ether:ethyl acetate 10:1) to provide tert-butyl 1-(4-chloro-2,3-difluorophenyl)cyclopropane-1-carboxylate (100 mg, 51% yield).

Step 3: Into a 100 mL round-bottom flask was added tert-butyl 1-(4-chloro-2,3-difluorophenyl)cyclopropane-1-carboxylate (150 mg, 0.52 mmol, 1 equiv) and trifluoroacetic acid (TFA) (1.50 mL), and dichloromethane (DCM) (2 mL) at room temperature. The mixture was stirred for 2 h at room temperature. Desired product could be detected by LCMS. The resulting mixture was concentrated under reduced pressure. This resulted in 1-(4-chloro-2,3-difluorophenyl)cyclopropane-1-carboxylic acid (130 mg, >100% yield). LCMS (ES, m/z): RT=0.82 min, m/z=231[M−1]⁻.

Step 4: Into a 40 mL round-bottom flask was added 1-(4-chloro-2,3-difluorophenyl)cyclopropane-1-carboxylic acid (“carboxyl (ii) reagent”) (130 mg, 0.55 mmol, 1 equiv), hydroxybenzotriazole (HOBT) (151 mg, 1.11 mmol, 2 equiv), 1-ethyl-3-(3-dimethylaminopropyl)carbodiimide (EDCI) (214.27 mg, 1.11 mmol, 2 equiv), diisopropylethyl amine (DIEA) (216.70 mg, 1.67 mmol, 3 equiv), dimethyl formamide (DMF) (0.50 mL) and 5-methyl-1-(1H-1,2,3,4-tetrazol-5-yl)azepan-3-amine (“amine (i) reagent”) (120.65 mg, 0.61 mmol, 1.1 equiv) at room temperature. The mixture was stirred for 2 h at room temperature. Desired product could be detected by LCMS. The residue was purified by reverse flash chromatography (C18 silica gel; mobile phase, acetonitrile (MeCN) in water, 10% to 50% gradient in 30 min; detector, UV 254 nm) to provide 1-(4-chloro-2,3-difluorophenyl)-N-(5-methyl-1-(1H-tetrazol-5-yl)azepan-3-yl)cyclopropane-1-carboxamide (Compound 4, rac-4) (110 mg, 48% yield). LCMS (ES, m/z): RT=0.83 min, m/z=411[M+1]⁺.

Step 5: The crude rac-4 (100 mg) was purified by Prep HPLC (Xselect CSH C18 OBD Column 30*150 mm 5 m; Mobile Phase A: acetonitrile (MeCN), Mobile Phase B: water (0.05% trifluoroacetic acid (TFA)); Flow rate: 60 mL/min; Gradient: 36% B to 46% B in 8 min, 46% B to 56% B in 12 min, 56% B; Wave Length: 254/220 nm) to provide an assumed mixture of trans isomers (25 mg; LCMS (ES, m/z): RT=0.63 min, m/z=411[M+1]⁺) as the first eluting peak and assumed cis mixture of isomers (18 mg, LCMS (ES, m/z): RT=0.66 min, m/z=411[M+1]⁺) as second eluting peak.

Step 6: The assumed trans mixture of isomers from step 5 was purified by PREP-CHIRAL HPLC (CHIRALART Cellulose-SB, 2*25 cm, 5 m; Mobile Phase A: Hexanes (0.1% trifluoroacetic acid (TFA)), Mobile Phase B: isopropanol (IPA): dichloromethane (DCM)=1:1; Flow rate: 20 mL/min; Gradient: 20% B to 20% B in 11 min; Wave Length: 254/220 nm) to provide Compound 4B* (RT(min): 9.05, 11.5 mg) and Compound 4C* (RT(min): 10.70, 9.6 mg). Stereochemistry was arbitrarily assigned.

Compound 4B*: LCMS (ES, m/z): LCMS RT=1.38 min, m/z=411[M+1]⁺; ¹H NMR (400 MHz, DMSO-d₆) δ 7.36 (d, J=8.7, 6.8, 1.8 Hz, 1H), 7.20 (d, J=8.9, 7.2, 2.0 Hz, 1H), 6.89 (d, J=7.7 Hz, 1H), 4.19 (q, J=7.1, 5.1 Hz, 1H), 3.55 (t, J=14.9, 9.7, 6.3 Hz, 3H), 3.24 (d, J=13.7, 8.8, 4.6 Hz, 1H), 1.82-1.75 (m, 1H), 1.71 (d, J=14.6, 6.8 Hz, 1H), 1.58-1.51 (m, 1H), 1.43 (t, J=13.5, 7.0, 6.2, 3.7 Hz, 3H), 1.32 (d, J=13.9, 9.0, 4.6 Hz, 1H), 1.11 (d, J=10.2, 6.2, 3.3 Hz, 1H), 0.99 (d, J=8.9, 6.3, 3.0 Hz, 1H), 0.87 (d, J=6.9 Hz, 2H).

Compound 4C*: LCMS (ES, m/z): RT=1.37 min, m/z=411[M+1]+; ¹H NMR (400 MHz, DMSO-d₆) δ 7.36 (d, J=8.8, 6.8, 1.9 Hz, 1H), 7.20 (d, J=9.0, 7.0, 2.0 Hz, 1H), 6.88 (s, 1H), 4.20 (d, J=6.0 Hz, 1H), 3.58 (d, J=14.9, 5.1 Hz, 3H), 3.52 (d, J=6.9 Hz, 1H), 1.79 (d, J=7.0 Hz, 1H), 1.69 (d, J=14.9 Hz, 1H), 1.59-1.51 (m, 1H), 1.43 (t, J=16.0, 6.1, 3.5 Hz, 3H), 1.32 (d, J=13.8, 8.9, 4.5 Hz, 1H), 1.11 (d, J=10.2, 6.3, 3.2 Hz, 1H), 0.99 (d, J=9.0, 6.3, 3.0 Hz, 1H), 0.87 (d, J=6.8 Hz, 3H).

Step 7: The assumed cis mixture of isomers from step 5 was purified by PREP-CHIRAL HPLC (CHIRAL ART Cellulose-SB, 2*25 cm, 5 m; Mobile Phase A: Hexanes (0.1% trifluoroacetic acid (TFA)), Mobile Phase B: isopropanol (IPA): dichloromethane (DCM)=1:1; Flow rate: 20 mL/min; Gradient: 12% B to 12% B in 20 min; Wave Length: 220/254 nm) to provide Compound 4A* (RT(min): 16.46, 5.2 mg) and Compound 4D* (RT(min): 18.42, 5.8 mg). Stereochemistry was arbitrarily assigned.

Compound 4A*: LCMS (ES, m/z): RT=1.53 min, m/z=411[M+1]⁺; ¹H NMR (400 MHz, DMSO-d₆) δ 7.47-7.32 (m, 1H), 7.29-7.20 (m, 1H), 3.58 (dd, J=14.3, 5.5 Hz, 1H), 3.53-3.47 (m, 1H), 3.45-3.40 (m, 1H), 3.33-3.27 (m, 1H), 1.72 (d, J=13.6 Hz, 1H), 1.54 (dd, J=13.3, 4.3 Hz, 1H), 1.44 (d, J=3.1 Hz, 1H), 1.37-1.22 (m, 1H), 1.07 (ddd, J=20.0, 12.8, 9.8 Hz, 2H), 0.88 (d, J=6.6 Hz, 2H).

Compound 4D*: LCMS (ES, m/z): RT=1.53 min, m/z=411[M+1]⁺; ¹H NMR (400 MHz, DMSO-d₆) δ 7.45-7.36 (m, 2H), 7.29-7.20 (m, 1H), 4.00 (s, 1H), 3.58 (dd, J=14.4, 5.5 Hz, 1H), 3.51 (d, J=13.6 Hz, 1H), 3.45-3.40 (m, 2H), 3.39 (s, 1H), 1.73 (d, J=14.1 Hz, 1H), 1.54 (d, J=12.8 Hz, 1H), 1.44 (d, J=3.1 Hz, 2H), 1.36-1.24 (m, 1H), 1.07 (ddd, J=19.8, 12.8, 9.8 Hz, 3H), 0.88 (d, J=6.7 Hz, 3H).

### Example 5. Synthesis of 1-(4-(difluoromethyl)phenyl)-N-(5-methyl-1-(1H-tetrazol-5-yl)azepan-3-yl)cyclopropane-1-carboxamide (Compound 5, rac-5) and Compounds 5A*, 5B*, 5C*, and 5D*

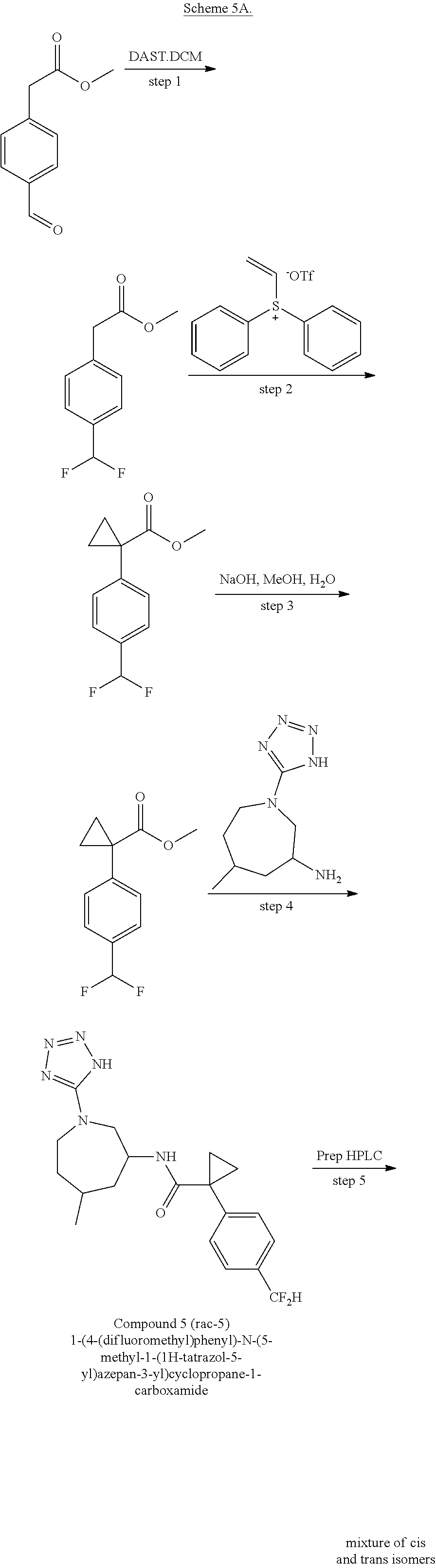

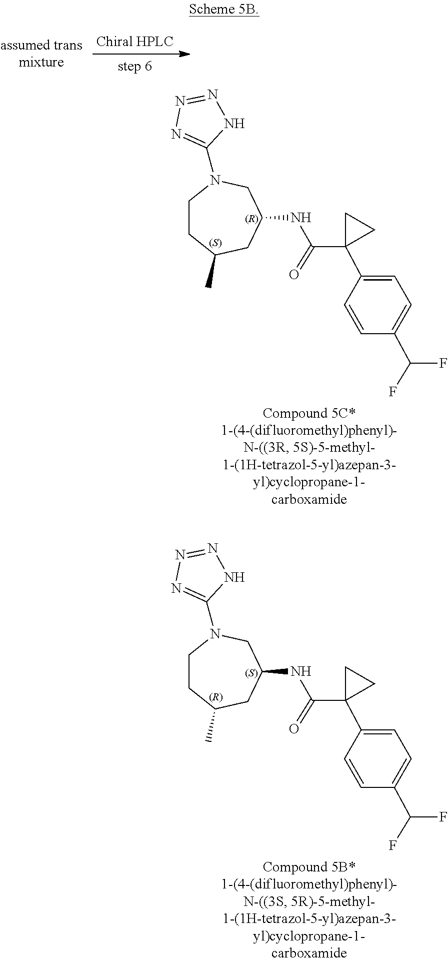

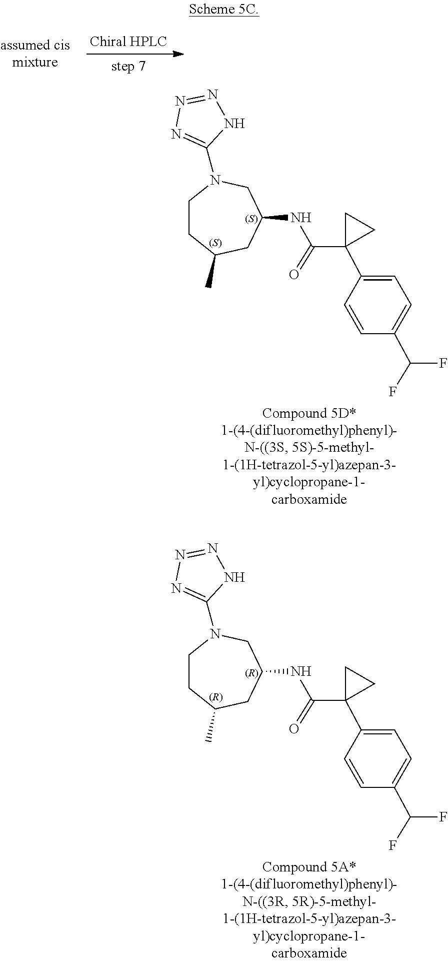

### Example 5 Follows Protocol A

Step 1: Into a 100 mL round-bottom flask was added methyl 2-(4-formylphenyl)acetate (1 g, 5.61 mmol, 1 equiv), diethylaminosulfur trifluoride (DAST) (17 mL, 0.105 mmol, 0.02 equiv), and dichloromethane (DCM) (20 mL) at 0° C. The mixture was stirred for 1 h at room temperature. Desired product could be detected by LCMS. The reaction was quenched with NaHCO₃(aq.) at room temperature.

The residue was purified by silica gel column chromatography, eluting with petroleum ether:ethyl acetate (1:1) to provide methyl 2-[4-(difluoromethyl)phenyl]acetate (800 mg, 71% yield).

Step 2: Into a 100 mL round-bottom flask was added methyl 2-[4-(difluoromethyl)phenyl]acetate (800 mg, 3.99 mmol, 1 equiv), 1,8-diazabicyclo[5.4.0]undec-7-ene (DBU) (3650.41 mg, 23.97 mmol, 6 equiv), dimethylsulfoxide (DMSO) (5 mL) and ethenyldiphenylsulfanium triflate (2896.36 mg, 7.99 mmol, 2 equiv) at room temperature. The mixture was stirred for 2 h at room temperature. Desired product could be detected by GCMS. The residue was purified by reverse flash chromatography (C18 silica gel; mobile phase, Acetonitrile (MeCN) in water, 10% to 50% gradient in 10 min; detector, UV 254 nm) to provide methyl 1-[4-(difluoromethyl) phenyl]cyclopropane-1-carboxylate (500 mg, 55% yield).

Step 3: Into a 50 mL round-bottom flask was added methyl 1-[4-(difluoromethyl)phenyl]cyclopropane-1-carboxylate (450 mg, 1.98 mmol, 1 equiv), methanol (MeOH) (5 mL), water (1 mL) and NaOH (795.62 mg, 19.89 mmol, 10 equiv) at room temperature. Desired product could be detected by LCMS. The resulting mixture was extracted with water (3×10 mL). Into above mixture was added HCl (aq)(10 mL, 1 mol/L) at room temperature. The resulting mixture was extracted with ethyl acetate (3×20 mL), and the layers were combined. After filtration, the filtrate dried over sodium sulfate and concentrated under reduced pressure. This resulted in 1-[4-(difluoromethyl)phenyl]cyclopropane-1-carboxylic acid (400 mg, 95% yield). LCMS (ES, m/z): RT=0.72 min, m/z=211[M−1]⁻.

Step 4: Into a 40 mL round-bottom flask was added 1-[4-(difluoromethyl)phenyl]cyclopropane-1-carboxylic acid (“carboxyl (ii) reagent”) (100 mg, 0.47 mmol, 1 equiv), diisopropylethyl amine (DIEA) (182.73 mg, 1.41 mmol, 3 equiv), dimethyl formamide (DMF) (4 mL) and 5-methyl-1-(1H-1,2,3,4-tetrazol-5-yl)azepan-3-amine (“amine (i) reagent”) (101.74 mg, 0.51 mmol, 1.1 equiv) at room temperature. The mixture was stirred for 1 h at room temperature. Desired product could be detected by LCMS. The residue was purified by reverse flash chromatography (C18 silica gel; mobile phase, Acetonitrile (MeCN) in water, 10% to 50% gradient in 30 min; detector, UV 254 nm) to provide 1-(4-(difluoromethyl)phenyl)-N-(5-methyl-1-(1H-tetrazol-5-yl)azepan-3-yl)cyclopropane-1-carboxamide (Compound 5, rac-5) (100 mg, 48% yield). LCMS (ES, m/z): RT=0.84 min, m/z=391[M+1]⁺.

Step 5: The crude product, rac-5, (100 mg) was purified by PREP HPLC (XBridge Prep Phenyl OBD Column, 19*150 mm, 5 m; Mobile Phase A: water(10 mmol/L NH₄HCO₃), Mobile Phase B: acetonitrile (MeCN); Flow rate: 60 mL/min; Gradient: 20% B to 28% B in 8 min, 28% B; Wave Length: 254 nm) to provide an assumed mixture of trans isomers (26 mg, LCMS (ES, m/z): RT=0.78 min, m/z=391[M+1]⁺) as first eluting peak and an assumed mixture of cis isomers (40 mg, LCMS (ES, m/z): RT=0.79 min, m/z=391[M+1]⁺) as second eluting peak, stereochemistry arbitrarily assigned.

Step 6: The assumed mixture of trans isomers of rac-5 were separated by Chiral HPLC (CHIRAL ART Cellulose-SC, 2*25 cm, 5 m; Mobile Phase A: Hexanes (0.1% trifluoroacetic acid (TFA)), Mobile Phase B: isopropanol (IPA): dichloromethane (DCM)=1:1; Flow rate: 20 mL/min; Gradient: 35% B to 35% B in 43 min; Wave Length: 254/220 nm) to provide Compound 5B* (RT(min): 29.35, 3.7 mg) and Compound 5C* (RT(min): 38.07, 5.6 mg). Stereochemistry was arbitrarily assigned.

Compound 5B*: LCMS (ES, m/z): RT=1.23 min, m/z=391[M+1]⁺; ¹H NMR (400 MHz, DMSO-d₆) δ 7.44 (d, J=8.0 Hz, 2H), 7.35 (d, J=8.0 Hz, 2H), 6.99 (s, 1H), 6.55 (d, J=8.2 Hz, 1H), 4.19 (dp, J=10.3, 5.1 Hz, 1H), 3.58 (d, J=1.5 Hz, 2H), 3.49 (dt, J=13.4, 4.7 Hz, 1H), 3.11 (ddd, J=13.7, 10.2, 4.2 Hz, 1H), 1.65 (dd, J=11.2, 7.0 Hz, 2H), 1.61-1.41 (m, 2H), 1.41-1.24 (m, 3H), 1.07-0.94 (m, 2H), 0.87 (d, J=6.7 Hz, 3H).

Compound 5C*: LCMS (ES, m/z): RT=0.73 min, m/z=391[M+1]⁺; ¹H NMR (400 MHz, DMSO-d₆) δ 7.44 (d, J=8.0 Hz, 2H), 7.35 (d, J=8.0 Hz, 2H), 6.55 (d, J=8.2 Hz, 1H), 4.19 (d, J=10.1, 5.0 Hz, 1H), 3.52-3.47 (m, 3H), 3.11 (d, J=13.7, 10.3, 4.2 Hz, 1H), 1.69-1.58 (m, 2H), 1.49 (d, J=20.1, 14.9, 10.2, 4.8 Hz, 2H), 1.42-1.22 (m, 3H), 1.09-0.94 (m, 2H), 0.87 (d, J=6.7 Hz, 3H).

Step 7: The assumed mixture of cis isomers of rac-5 were separated by Chiral HPLC (LUX 5 um Cellulose-4, 2.12*25 cm, 5 m; Mobile Phase A: Hexanes (0.1% trifluoroacetic acid (TFA)), Mobile Phase B: isopropanol (IPA); Flow rate: 20 mL/min; Gradient: 50% B to 50% B in 13.5 min; Wave Length: 254/220 nm) to provide Compound 5A* (RT(min): 11.63, 4.3 mg) and Compound 5D* (RT(min): 8.02, 7.2 mg). Stereochemistry was arbitrarily assigned.

Compound 5A* LCMS (ES, m/z): RT=1.40 min, m/z=391[M+1]⁺; ¹H NMR (400 MHz, DMSO-d₆) δ 7.55 (d, J=8.0 Hz, 2H), 7.48 (d, J=8.0 Hz, 2H), 7.20-6.88 (m, 2H), 3.96 (d, J=12.0, 3.7 Hz, 1H), 3.57 (d, J=14.1, 5.2 Hz, 1H), 3.41 (d, J=24.4, 18.7, 14.4, 9.9, 4.6 Hz, 3H), 1.74 (d, J=13.4, 3.9 Hz, 1H), 1.52 (td, J=11.6, 10.5, 6.1 Hz, 2H), 1.41-1.31 (m, 2H), 1.29-1.12 (m, 2H), 1.08-0.97 (m, 2H), 0.88 (d, J=6.6 Hz, 3H).

Compound 5D* LCMS (ES, m/z): RT=1.39 min, m/z=391[M+1]⁺; ¹H NMR (400 MHz, DMSO-d₆) δ 7.55 (d, J=8.0 Hz, 2H), 7.48 (d, J=8.0 Hz, 2H), 7.19-6.87 (m, 2H), 3.97 (d, J=11.3, 4.1 Hz, 1H), 3.57 (d, J=14.1, 5.2 Hz, 2H), 3.46-3.34 (m, 2H), 1.75 (d, J=14.2, 4.0 Hz, 1H), 1.52 (td, J=11.3, 9.8, 5.9 Hz, 2H), 1.45-1.13 (m, 4H), 1.08-0.97 (m, 2H), 0.88 (d, J=6.7 Hz, 3H).

### Example 6. Synthesis of N-(5-methyl-1-(1H-tetrazol-5-yl)azepan-3-yl)-1-(3-methyl-4-(trifluoromethyl)phenyl)cyclopropane-1-carboxamide (Compound 6, rac-6) and Compounds 6A*, 6B*, 6C*, and 6D*

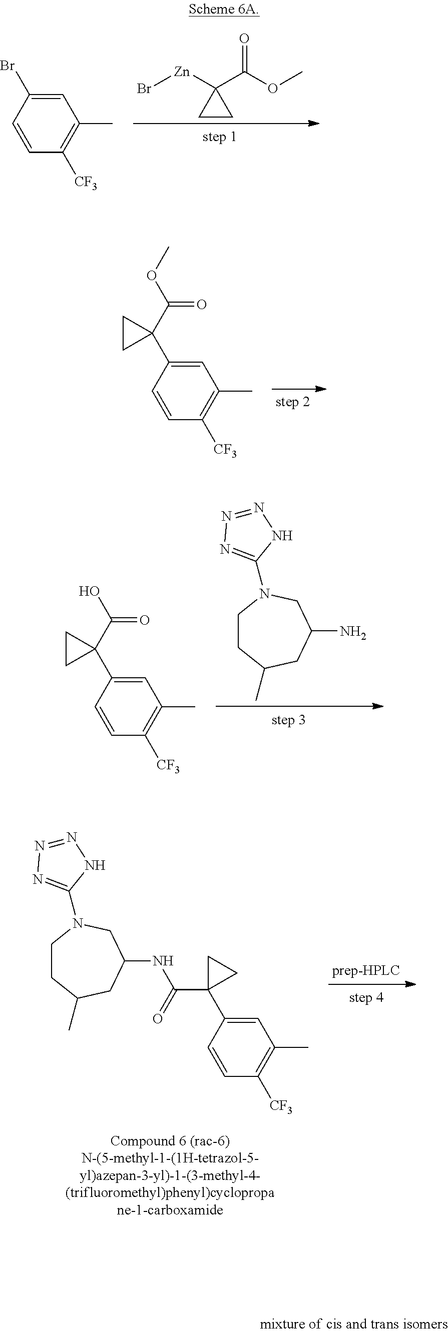

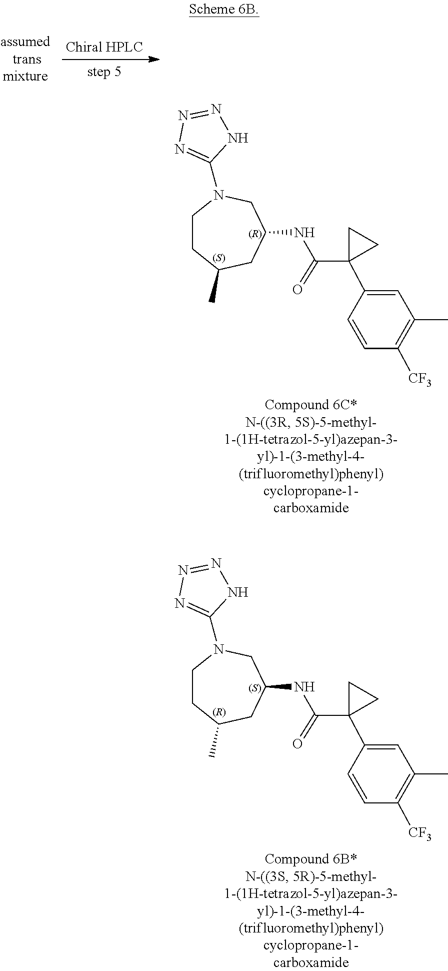

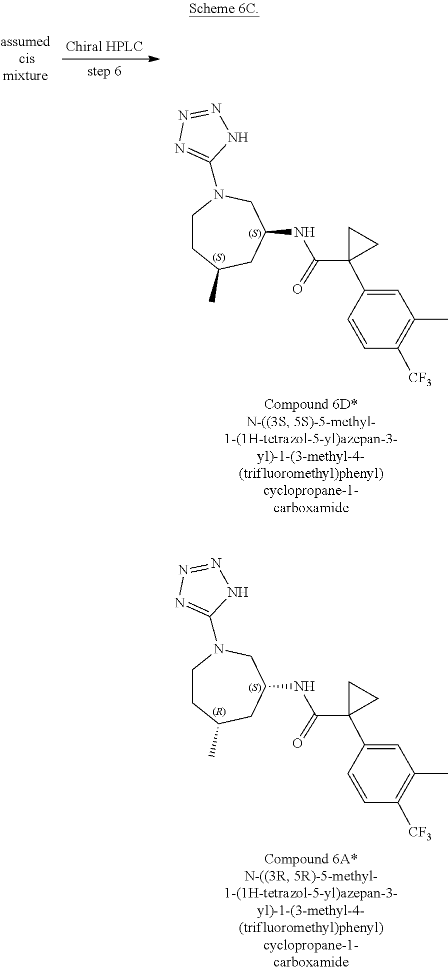

### Example 6 Follows Protocol A

Step 1: Into a 20 mL vial was added 4-bromo-2-methyl-1-(trifluoromethyl) benzene (700 mg, 2.92 mmol, 1 equiv), bis(tri-tert-butylphosphine)palladium(0) (Pd[(t-Bu)₃P]₂) (448.98 mg, 0.87 mmol, 0.3 equiv), bromo[1-(methoxycarbonyl)cyclopropyl]zinc (7156.82 mg, 29.28 mmol, 10 equiv), tetrahydrofuran (THF) (7 mL). The resulting mixture was stirred overnight at 60° C. under nitrogen atmosphere. Desired product could be detected by LCMS. The resulting mixture was extracted with ethyl acetate (EtOAc) (2×30 mL). The combined organic layers were washed with water (1×10 mL) and dried over anhydrous Na₂SO₄. After filtration, the filtrate was concentrated under reduced pressure. The residue was purified by Prep-TLC (petroleum ether:ethyl acetate 5:1) to provide methyl 1-[3-methyl-4-(trifluoromethyl) phenyl]cyclopropane-1-carboxylate (360 mg, 43% yield). LCMS:(ES, m/z): RT=1.05 min, m/z=259.0[M+1]⁺.

Step 2: Into a 20 mL vial was added methyl 1-[3-methyl-4-(trifluoromethyl) phenyl]cyclopropane-1-carboxylate (350 mg, 1.35 mmol, 1 equiv), LiOH (194.76 mg, 8.13 mmol, 6 equiv), methanol (MeOH) (3.5 mL), and water (0.7 mL). The resulting mixture was stirred for 1 h at room temperature. Desired product could be detected by LCMS. The mixture was acidified to pH 3 with HCl (aq.). The resulting mixture was extracted with ethyl acetate (EtOAc) (3×20 mL). The combined organic layers were washed with water (1×10 mL), and dried over anhydrous Na₂SO₄. After filtration, the filtrate was concentrated under reduced pressure. This resulted in 1-[3-methyl-4-(trifluoromethyl) phenyl]cyclopropane-1-carboxylic acid (200 mg, 51% yield). LCMS:(ES, m/z): RT=1.07 min, m/z=245.0[M+1]⁺.

Step 3: Into an 8 mL vial was added 1-[3-methyl-4-(trifluoromethyl) phenyl]cyclopropane-1-carboxylic acid (“carboxyl (ii) reagent”) (150 mg, 0.61 mmol, 1 equiv), hydroxybenzotriazole (HOBT) (165.99 mg, 1.22 mmol, 2 equiv), 1-ethyl-3-(3-dimethylaminopropyl)carbodiimide (EDCI) (235.49 mg, 1.22 mmol, 2 equiv), diisopropylethyl amine (DIEA) (238.16 mg, 1.84 mmol, 3 equiv), dimethyl formamide (DMF) (1.5 mL), and 5-methyl-1-(1H-1,2,3,4-tetrazol-5-yl) azepan-3-amine (“amine (i) reagent”) (120.55 mg, 0.61 mmol, 1 equiv). The resulting mixture was stirred for 1 h at room temperature. Desired product could be detected by LCMS. The resulting mixture was concentrated under reduced pressure. The residue was purified by reverse flash chromatography (C18 silica gel; mobile phase, Acetonitrile (MeCN) in water, 40% to 50% gradient in 10 min; detector, UV 254 nm) to provide the crude product, N-(5-methyl-1-(1H-tetrazol-5-yl)azepan-3-yl)-1-(3-methyl-4-(trifluoromethyl)phenyl)cyclopropane-1-carboxamide (Compound 6, rac-6), (140 mg, 80% purity).

Step 4: The crude product, rac-6, was purified by PREP HPLC (Xselect CSH C18 OBD Column 30*150 mm 5 um; mobile phase, water (0.05% trifluoroacetic acid (TFA)) and acetonitrile (MeCN) (40% acetonitrile (MeCN) up to 50% in 10 min, up to 60% in 2 min); Detector, UV 254 nm) to provide an assumed mixture of cis isomers (second eluting peak) and assumed mixture of trans isomers (first eluting peak). Stereochemistry was arbitrarily assigned.

Step 5: The assumed trans mixture (26 mg) was purified by Chiral-PREP HPLC (Lux 5 um Cellulose-4, 2.12*25 cm, 5 m; Mobile Phase A: Hexanes (0.1% trifluoroacetic acid), Mobile Phase B: isopropanol (IPA); Flow rate: 20 mL/min; Gradient: 50% B to 50% B in 10 min; Wave Length: 220/254 nm) to provide Compound 6B* (RT(min): 6.93, 4.90 mg) and Compound 6C* (RT(min): 8.82, 6.20 mg). Stereochemistry was arbitrarily assigned.

Compound 6B*: LCMS:(ES, m/z): RT=0.80 min, m/z=423.0[M+H]⁺. ¹H NMR (400 MHz, Methanol-d₄) δ 7.50 (d, J=8.0 Hz, 1H), 7.27-7.19 (m, 2H), 4.21 (s, 1H), 3.85-3.75 (m, 1H), 3.61-3.52 (m, 1H), 3.50-3.40 (m, 1H), 3.20-3.08 (m, 1H), 2.46-2.40 (m, 3H), 1.77 (t, J=17.0 Hz, 2H), 1.71-1.36 (m, 4H), 1.19-1.14 (m, 1H), 1.15-1.10 (m, 1H), 1.10-1.01 (m, 1H), 0.99 (d, J=6.6 Hz, 3H).

Compound 6C*: LCMS:(ES, m/z): RT=0.80 min, m/z=423.0[M+H]⁺. ¹H NMR (400 MHz, Methanol-d₄) δ 7.50 (d, J=8.0 Hz, 1H), 7.27-7.19 (m, 2H), 4.22 (d, J=5.6 Hz, 1H), 3.85-3.75 (m, 1H), 3.61-3.52 (m, 1H), 3.50-3.40 (m, 1H), 3.20-3.08 (m, 1H), 2.46-2.41 (m, 3H), 1.84-1.71 (m, 2H), 1.71-1.60 (m, 1H), 1.63-1.37 (m, 3H), 1.19-1.15 (m, 1H), 1.14-1.10 (m, 1H), 1.10-1.01 (m, 1H), 0.99 (d, J=6.6 Hz, 3H).

Step 6: The assumed cis mixture of isomers (19 mg) was purified by Chiral-PREP HPLC (LUX 5 um Cellulose-4, 2.12*25 cm, 5 um; mobile phase, Hexanes (0.1% trifluoroacetic acid (TFA)) and isopropanol (IPA) (hold 50% isopropanol (IPA) in 12 min); Detector, UV 254 nm) to provide Compound 6D* (RT(min): 7.83, 2.0 mg) and Compound 6A* (RT(min): 9.78, 2.4 mg). Stereochemistry was arbitrarily assigned.

Compound 6A*: LCMS:(ES, m/z): RT=1.31 min, m/z=423.0[M+1]⁺; ¹H NMR (400 MHz, DMSO-d₆) δ 14.69 (s, 1H), 7.62 (d, J=8.1 Hz, 1H), 7.39 (s, 1H), 7.34 (d, J=8.1 Hz, 1H), 7.24 (s, 1H), 3.98 (s, 1H), 3.63-3.54 (m, 1H), 3.54-3.38 (m, 2H), 3.29 (s, 1H), 2.45 (d, J=2.0 Hz, 3H), 1.73 (d, J=13.9 Hz, 1H), 1.55 (d, J=13.7 Hz, 2H), 1.44-1.36 (m, 1H), 1.40-1.28 (m, 1H), 1.24 (s, 1H), 1.22-1.08 (m, 1H), 1.04 (d, J=2.3 Hz, 2H), 0.88 (d, J=6.7 Hz, 3H).

Compound 6D*: LCMS:(ES, m/z): RT=1.31 min, m/z=423.0[M+1]⁺; ¹H NMR (400 MHz, Methanol-d₄) δ 7.65 (d, J=8.1 Hz, 1H), 7.44-7.35 (m, 2H), 4.02 (s, 1H), 3.60 (s, 1H), 3.61-3.45 (m, 2H), 3.49-3.37 (m, 1H), 2.54-2.48 (m, 3H), 1.83 (d, J=14.4 Hz, 1H), 1.70 (d, J=13.9 Hz, 1H), 1.64 (s, 1H), 1.62-1.42 (m, 3H), 1.33 (d, J=17.2 Hz, 1H), 1.28-1.16 (m, 1H), 1.19-1.09 (m, 2H), 0.97 (d, J=6.7 Hz, 3H).

### Example 7. Synthesis of 1-(2,5-difluoro-4-(trifluoromethyl)phenyl)-N-(5-methyl-1-(1H-tetrazol-5-yl)azepan-3-yl)cyclopropane-1-carboxamide (Compound 7, rac-7) and Compounds 7A*, 7B*, 7C*, and 7D*

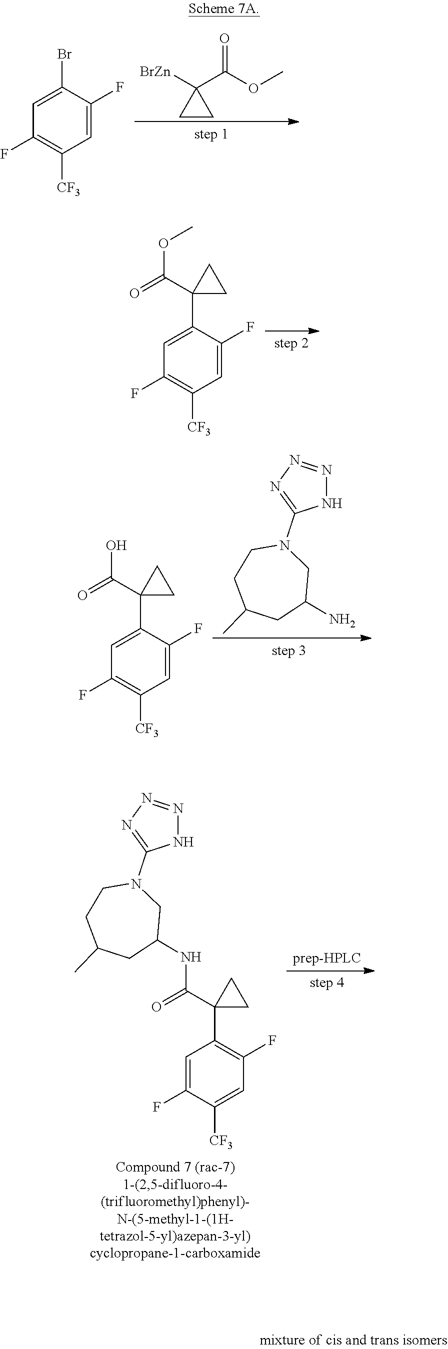

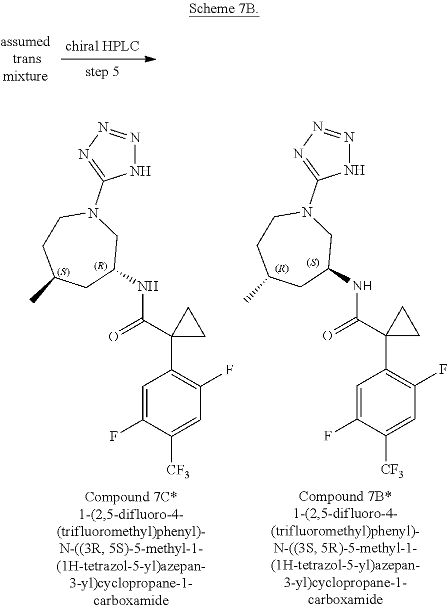

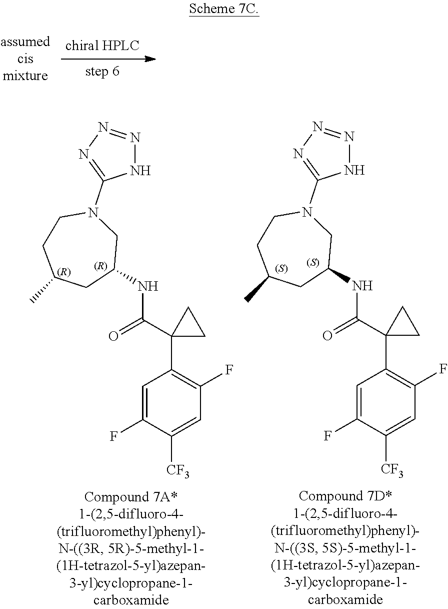

### Example 7 Follows Protocol A

Step 1: Into a 20 mL vial was added 1-bromo-2,5-difluoro-4-(trifluoromethyl)benzene (500 mg, 1.916 mmol, 1 equiv), bis(tri-tert-butylphosphine)palladium(0) (Pd[(t-Bu)₃P]₂) (293.73 mg, 0.575 mmol, 0.3 equiv) bromo[1-(methoxycarbonyl)cyclopropyl]zinc (9 mL, 28.740 mmol, 15 equiv), and tetrahydrofuran (THF) (0.5 mL). The resulting mixture was stirred overnight at 60° C. under nitrogen atmosphere. Desired product could be detected by GCMS. The resulting mixture was filtered, and the filter cake was washed with dichloromethane (DCM) (3×3 mL). The filtrate was concentrated under reduced pressure. The residue was purified by Prep-TLC (petroleum ether:ethyl acetate 7:1) to provide methyl 1-[2,5-difluoro-4-(trifluoromethyl)phenyl]cyclopropane-1-carboxylate (500 mg, 93% yield).

Step 2: Into a 40 mL vial was added methyl 1-[2,5-difluoro-4-(trifluoromethyl)phenyl]cyclopropane-1-carboxylate (500 mg, 1.784 mmol, 1 equiv), NaOH (214.12 mg, 5.352 mmol, 3 equiv), methanol (MeOH) (10 mL) and water (2 mL). The resulting mixture was stirred overnight at 40° C. under air atmosphere. Desired product could be detected by LCMS. The resulting liquid was dried under vacuum. The mixture was acidified to pH 2 with conc. HCl to provide 1-[2,5-difluoro-4-(trifluoromethyl)phenyl]cyclopropane-1-carboxylic acid (450 mg, 95% yield). The crude product was used in the next step directly without further purification.

Step 3: Into an 8 mL vial was added 1-[2,5-difluoro-4-(trifluoromethyl)phenyl]cyclopropane-1-carboxylic acid (“carboxyl (ii) reagent”) (150 mg, 0.564 mmol, 1 equiv), (1-[bis(dimethylamino)methylene]-1H-1,2,3-triazolo[4,5-b]pyridinium 3-oxide hexafluorophosphate (HATU) (214.28 mg, 0.564 mmol, 1 equiv), triethylamine (TEA) (171.08 mg, 1.692 mmol, 3 equiv), and dimethyl formamide (DMF) (1.5 mL). To the mixture was added 5-methyl-1-(1H-1,2,3,4-tetrazol-5-yl)azepan-3-amine (“amine (i) reagent”) (132.72 mg, 0.677 mmol, 1.2 equiv) in portions at room temperature. The resulting mixture was stirred for 2 h at room temperature. Desired product could be detected by LCMS. The resulting oil was dried under vacuum and the residue was purified by reverse flash chromatography (C18 silica gel; mobile phase, acetonitrile (MeCN): water=1:1) to provide 1-(2,5-difluoro-4-(trifluoromethyl)phenyl)-N-(5-methyl-1-(1H-tetrazol-5-yl)azepan-3-yl)cyclopropane-1-carboxamide (Compound 7, rac-7).

Step 4: The crude product, rac-7, was purified by PREP HPLC (Xselect CSH F-Phenyl OBD column, 19*250 mm, 5 m; Mobile Phase A: water (0.05% trifluoroacetic acid (TFA), Mobile Phase B: Methanol (MeOH); Flow rate: 20 mL/min; Gradient: 56% B to 61% B in 8 min, 61% B; Wave Length: 254 nm) to provide an assumed mixture of cis isomers (30 mg), as first eluting peak and an assumed mixture of trans isomers (40 mg) as second eluting peak. Stereochemistry of cis and trans isomers were assigned arbitrarily.

Step 5: The assumed trans mixture (40 mg) was purified by PREP-CHIRAL HPLC (Lux 5 um Cellulose-4, 2.12*25 cm, 5 m Mobile Phase A: Hexanes (0.1% trifluoroacetic acid), Mobile Phase B: isopropanol (IPA); Flow rate: 20 mL/min; Gradient: 25% B to 25% B in 13 min; Wave Length: 254/220 nm) to provide Compound 7B* (RT(min): 8.99, 7.8 mg) and Compound 7C* (RT(min): 12.16, 8.4 mg). Stereochemistry was arbitrarily assigned.

Compound 7B*: LCMS:(ES, m/z): 1.59 min, m/z=445.1[M+H]⁺; ¹H NMR (400 MHz, Methanol-d₄) δ 7.46-7.33 (m, 2H), 4.25-4.17 (m, 1H), 3.75-3.67 (m, 1H), 3.32-3.27 (m, 1H), 1.90-1.73 (m, 3H), 1.69-1.55 (m, 3H), 1.54-1.45 (m, 1H), 1.26-1.19 (m, 1H), 1.14-1.07 (m, 1H), 0.98 (d, J=6.8 Hz, 3H).

Compound 7C*: LCMS:(ES, m/z): 1.59 min, m/z=445.1[M+H]⁺; ¹H NMR (400 MHz, Methanol-d₄) δ 7.46-7.33 (m, 2H), 4.25-4.17 (m, 1H), 3.75-3.67 (m, 1H), 3.32-3.27 (m, 1H), 1.90-1.73 (m, 3H), 1.69-1.55 (m, 3H), 1.54-1.45 (m, 1H), 1.26-1.19 (m, 1H), 1.14-1.07 (m, 1H), 0.98 (d, J=6.8 Hz, 3H).

Step 6: The assumed cis mixture (30 mg) was purified by PREP-CHIRAL HPLC (CHIRALPAK IF, 2*25 cm, 5 m; Mobile Phase A: Hexanes (0.1% trifluoroacetic acid), Mobile Phase B: ethanol; Flow rate: 20 mL/min; Gradient: 5% B to 5% B in 30 min; Wave Length: 254/220 nm) to provide Compound 7A* (RT(min): 18.82, 5.0 mg) and Compound 7D* (RT(min): 22.40, 4.0 mg). Stereochemistry was arbitrarily assigned.

Compound 7A*: LCMS: (ES, m/z): 1.05 min, m/z=445.2[M+H]⁺; ¹H NMR (400 MHz, Methanol-d₄) δ 7.55-7.40 (m, 2H), 4.11-4.00 (m, 1H), 3.67-3.56 (m, 2H), 3.56-3.49 (m, 1H), 3.47-3.37 (m, 1H), 1.82 (d, J=14.2 Hz, 1H), 1.77-1.69 (m, 1H), 1.64 (d, J=3.1 Hz, 2H), 1.52-1.36 (m, 1H), 1.26-1.10 (m, 3H), 0.98 (d, J=6.7 Hz, 3H).

Compound 7D*: LCMS:(ES, m/z): 1.05 min, m/z=445.2[M+H]⁺; ¹H NMR (400 MHz, Methanol-d₄) δ 7.55-7.40 (m, 2H), 4.11-4.00 (m, 1H), 3.67-3.56 (m, 2H), 3.56-3.49 (m, 1H), 3.47-3.37 (m, 1H), 1.82 (d, J=14.2 Hz, 1H), 1.77-1.69 (m, 1H), 1.64 (d, J=3.1 Hz, 2H), 1.52-1.36 (m, 1H), 1.26-1.10 (m, 3H), 0.98 (d, J=6.7 Hz, 3H).

### Example 8. Synthesis of 1-(4-cyclobutylphenyl)-N-(5-methyl-1-(1H-tetrazol-5-yl)azepan-3-yl)cyclopropane-1-carboxamide (Compound 8, rac-8), and Compounds 8A*, 8B*, 8C*, and 8D*

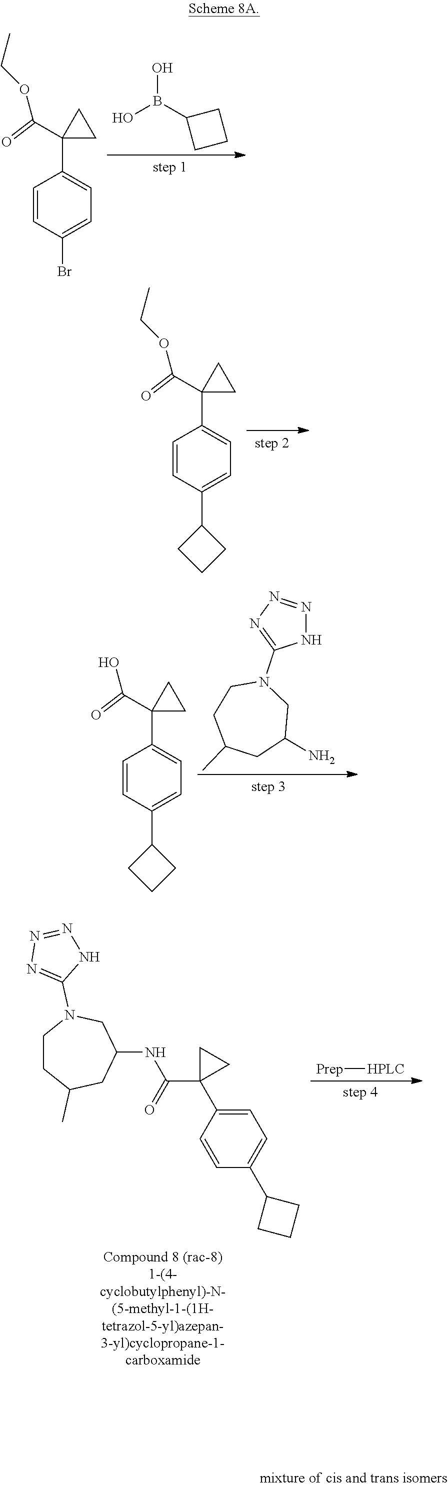

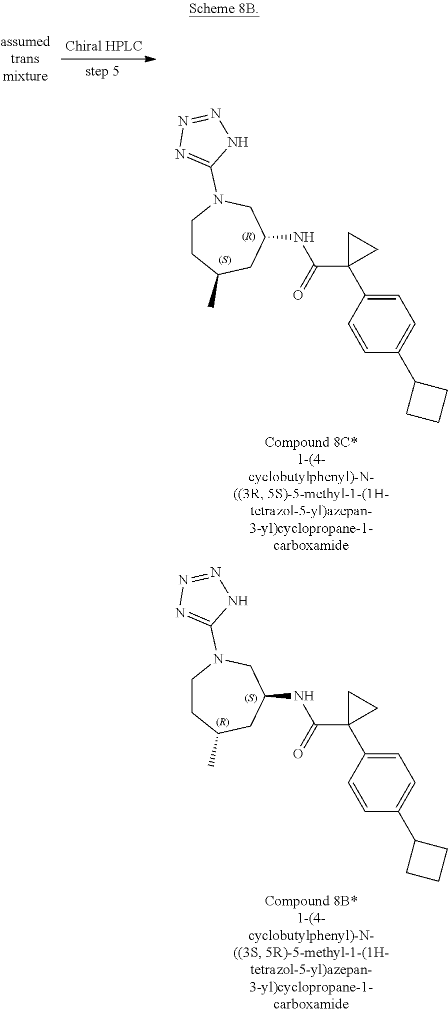

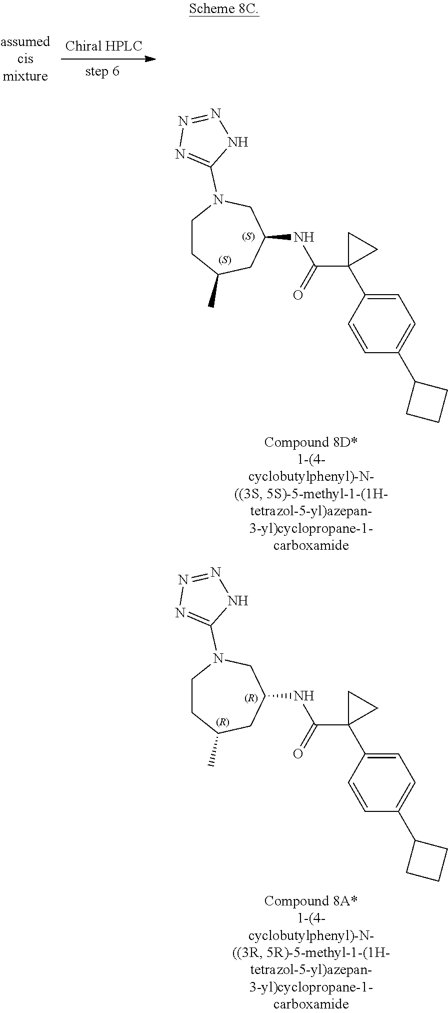

### Example 8 Follows Protocol A

Step 1: Into a 20 mL vial was added ethyl 1-(4-bromophenyl)cyclopropane-1-carboxylate (600 mg, 2.22 mmol, 1 equiv), cyclobutylboronic acid (668.27 mg, 6.68 mmol, 3 equiv),4-(anthracen-9-yl)-3-tert-butyl-2H-1,3-benzoxaphosphole (165.16 mg, 0.44 mmol, 0.2 equiv), K₃PO₄ (1419.63 mg, 6.68 mmol, 3 equiv), palladium (II) acetate (Pd(OAc)₂) (50.05 mg, 0.22 mmol, 0.1 equiv), and toluene (5 mL). The resulting mixture was stirred for 2 h at 100° C. under nitrogen atmosphere. Desired product could be detected by LCMS. The resulting mixture was extracted with ethyl acetate (EtOAc) (2×30 mL). The combined organic layers were washed with water (1×20 mL), dried over anhydrous Na₂SO₄. After filtration, the filtrate was concentrated under reduced pressure. The residue was purified by silica gel column chromatography, eluting with dichloromethane (DCM)/petroleum ether (9:1), to provide ethyl 1-(4-cyclobutylphenyl) cyclopropane-1-carboxylate (410 mg, 67% yield). LCMS:(ES, m/z): RT=1.19 min, m/z=245.0[M+1]⁺.

Step 2: Into an 8 mL vial was added ethyl 1-(4-cyclobutylphenyl) cyclopropane-1-carboxylate (400 mg, 1.63 mmol, 1 equiv), methanol (MeOH) (4 mL), NaOH (327.40 mg, 8.18 mmol, 5 equiv), and water (1.25 mL). The resulting mixture was stirred for 2 h at 40° C. Desired product could be detected by LCMS. The mixture was acidified to pH3 with HCl (aq.). The resulting mixture was extracted with ethyl acetate (EtOAc) (3×10 mL). The combined organic layers were washed with water (1×10 mL) and dried over anhydrous Na₂SO₄. After filtration, the filtrate was concentrated under reduced pressure. This resulted in 1-(4-cyclobutylphenyl) cyclopropane-1-carboxylic acid (280 mg, 68% yield). LCMS:(ES, m/z): RT=0.70 min, m/z=217.0[M+1]⁺.

Step 3: Into an 8 mL vial was added 1-(4-cyclobutylphenyl)cyclopropane-1-carboxylic acid (“carboxyl (ii) reagent”) (270 mg, 1.24 mmol, 1 equiv), 5-methyl-1-(1H-1,2,3,4-tetrazol-5-yl)azepan-3-amine (“amine (i) reagent”) (245 mg, 1.24 mmol, 1 equiv), hydroxybenzotriazole (HOBT) (337.38 mg, 2.49 mmol, 2 equiv), diisopropylethyl amine (DIEA) (484.05 mg, 3.74 mmol, 3 equiv), 1-ethyl-3-(3-dimethylaminopropyl)carbodiimide (EDCI) (478.63 mg, 2.49 mmol, 2 equiv), and dimethyl formamide (DMF) (2.7 mL). The resulting mixture was stirred for 1 h at room temperature. Desired product could be detected by LCMS. The resulting mixture was concentrated under reduced pressure. The residue was purified by reverse flash chromatography (C18 silica gel; mobile phase, acetonitrile (MeCN) in water, 40% to 50% gradient in 10 min; detector, UV 254 nm) to provide crude product, 1-(4-cyclobutylphenyl)-N-(5-methyl-1-(1H-tetrazol-5-yl)azepan-3-yl)cyclopropane-1-carboxamide (Compound 8, rac-8) (240 mg, 80% yield) as a mixture of cis and trans isomers.

Step 4: The crude product, rac-8, was purified by PREP HPLC (XBridge Prep Phenyl OBD Column, 19*250 mm, 5 μm; mobile phase, water (0.1% trifluoroacetic acid (TFA)) and acetonitrile (MeCN) (43% acetonitrile (MeCN) up to 53% in 10 min); Detector, UV 254 nm) to provide a mixture of assumed trans isomers (30 mg) as first eluting peak and assumed cis isomers (25 mg) as second eluting peak. Stereochemistry was arbitrarily assigned.

Step 5: The assumed trans mixture (30 mg) was purified by Chiral-PREP HPLC (CHIRAL ART Cellulose-SB, 2*25 cm, 5 gm; Mobile Phase A: Hexanes (0.1% trifluoroacetic acid), Mobile Phase B: ethanol (EtOH): dichloromethane (DCM)=1:1; Flow rate: 20 mL/min; Gradient: 15% B to 15% B in 10 min; Wave Length: 220/254 nm) to provide Compound 8B* (RT(min): 7.10, 9.7 mg) and Compound 8C* (RT(min): 8.35, 9.8 mg). Stereochemistry was arbitrarily assigned.

Compound 8B*: LCMS:(ES, m/z): RT=0.84 min, m/z=395.0[M+H]⁺. ¹H NMR (400 MHz, Methanol-d₄) δ 7.12 (d, J=8.2 Hz, 2H), 7.05 (d, J=8.0 Hz, 2H), 4.22-4.14 (m, 1H), 3.85-3.75 (m, 1H), 3.60-3.50 (m, 2H), 3.42-3.35 (m, 1H), 3.10-2.99 (m, 1H), 2.42-2.30 (m, 2H), 2.20-2.03 (m, 3H), 1.94-1.85 (m, 1H), 1.79-1.69 (m, 2H), 1.69-1.56 (m, 1H), 1.56-1.44 (m, 2H), 1.43-1.28 (m, 2H), 1.10-1.00 (m, 2H), 0.97 (d, J=6.1 Hz, 3H).

Compound 8C*: LCMS:(ES, m/z): RT=0.84 min, m/z=395.0[M+H]⁺. ¹H NMR (400 MHz, Methanol-d₄) δ 7.15-7.09 (m, 2H), 7.05 (d, J=8.1 Hz, 2H), 4.22-4.14 (m, 1H), 3.83-3.74 (m, 1H), 3.59-3.49 (m, 2H), 3.44-3.36 (m, 1H), 3.09-2.99 (m, 1H), 2.41-2.31 (m, 2H), 2.21-2.03 (m, 3H), 1.94-1.85 (m, 1H), 1.81-1.70 (m, 2H), 1.69-1.58 (m, 1H), 1.54-1.43 (m, 2H), 1.41-1.32 (m, 2H), 1.08-1.01 (m, 2H), 0.97 (d, J=6.3 Hz, 3H).

Step 6: The assumed cis mixture (25 mg) was purified by Chiral-PREP HPLC (Lux 5 um Cellulose-4, 2.12*25 cm, 5 m; Mobile Phase A: Hexanes (0.1% trifluoroacetic acid), Mobile Phase B: methanol:ethanol=1:1; Flow rate: 20 mL/min; Gradient: 40% B to 40% B in 7.2 min; Wave Length: 220/254 nm) to provide Compound 8D* (RT(min): 7.22, 8.4 mg) and Compound 8A*(RT(min): 8.52, 9.9 mg). Stereochemistry was arbitrarily assigned.

Compound 8A*: LCMS:(ES, m/z): RT=1.48 min, m/z=395.0[M+1]⁺. ¹H NMR (400 MHz, Methanol-d₄) δ 7.36-7.29 (m, 2H), 7.26 (d, J=8.1 Hz, 2H), 4.03-3.92 (m, 1H), 3.65-3.50 (m, 3H), 3.50-3.40 (m, 2H), 2.43-2.31 (m, 2H), 2.26-2.12 (m, 2H), 2.15-1.99 (m, 1H), 1.96-1.80 (m, 2H), 1.66 (s, 1H), 1.58-1.43 (m, 3H), 1.31 (s, 1H), 1.28-1.14 (m, 1H), 1.14-1.03 (m, 2H), 0.96 (d, J=6.7 Hz, 3H).

Compound 8D*: LCMS:(ES, m/z): RT=1.04 min, m/z=395.0[M+1]+. H NMR (400 MHz, Methanol-d₄) δ 7.37-7.29 (m, 2H), 7.29-7.22 (m, 2H), 4.03-3.92 (m, 1H), 3.65-3.51 (m, 3H), 3.51-3.40 (m, 2H), 2.43-2.31 (m, 2H), 2.26-2.12 (m, 2H), 2.15-1.99 (m, 1H), 1.96-1.80 (m, 1H), 1.72-1.59 (m, 2H), 1.58-1.43 (m, 3H), 1.34 (d, J=22.9 Hz, 1H), 1.28-1.14 (m, 1H), 1.14-1.03 (m, 2H), 0.96 (d, J=6.8 Hz, 3H).

### Example 9. Synthesis of N-(9-(1H-tetrazol-5-yl)-9-azabicyclo[4.2.1]nonan-2-yl)-1-(4-chlorophenyl)cyclopropane-1-carboxamide (Compound 9, rac-9), and 9A*, 9B*, 9C*, and 9D*

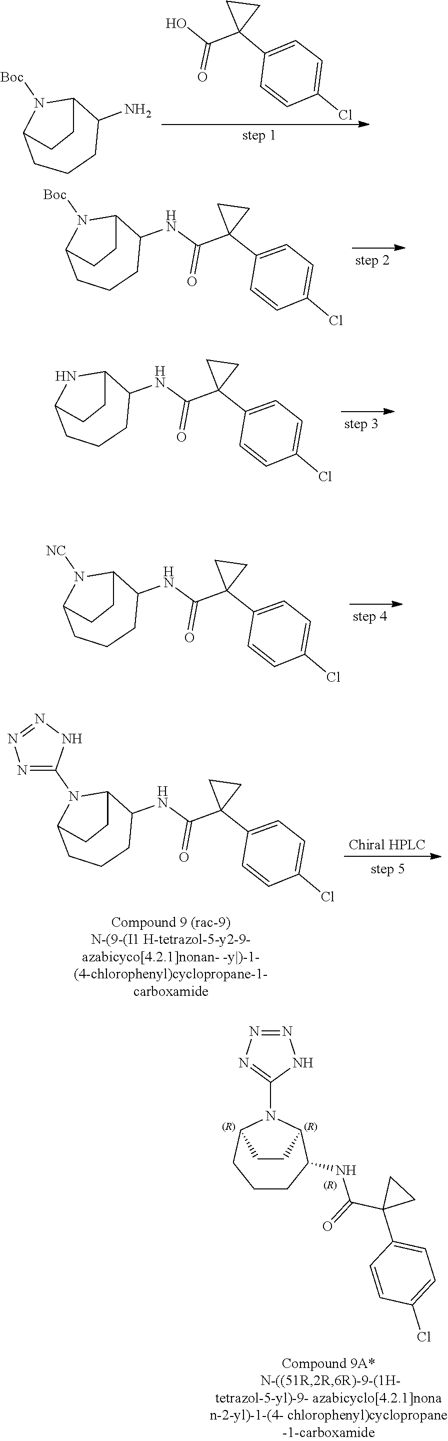

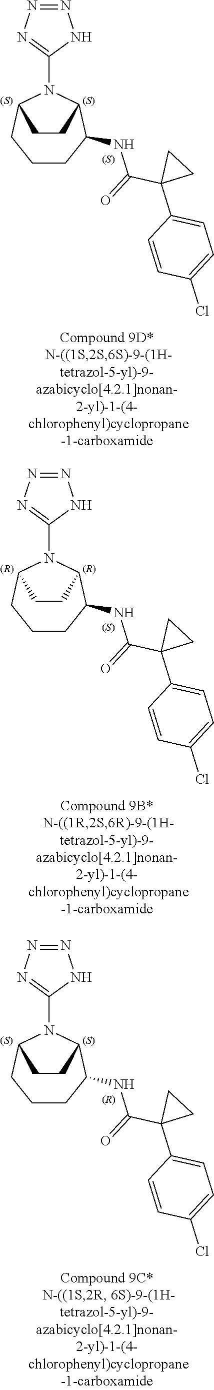

### Example 9 Follows Protocol C

Step 1: Into a 40 mL vial was added tert-butyl 2-amino-9-azabicyclo[4.2.1]nonane-9-carboxylate (“amine (iv) reagent”) (500 mg, 2.080 mmol, 1 equiv) and 1-(4-chlorophenyl)cyclopropane-1-carboxylic acid (“carboxyl (ii) reagent”) (449.96 mg, 2.288 mmol, 1.1 equiv), N,N,N′,N′-tetramethylichloroformamidinium hexafluorophosphate (TCFH) (1167.40 mg, 4.160 mmol, 2 equiv), N-methyl imidazole (NMI) (341.61 mg, 4.160 mmol, 2 equiv), acetonitrile (MeCN) (15 mL) at room temperature. The resulting mixture was stirred for 1 h at room temperature under nitrogen atmosphere.

The residue was purified by reverse flash chromatography (C18 silica gel; mobile phase, acetonitrile (MeCN) in water (10 mmol/L NH₄HCO₃), 10% to 50% gradient in 10 min; detector, UV 254 nm) to provide tert-butyl 2-(1-(4-chlorophenyl)cyclopropane-1-carboxamido)-9-azabicyclo[4.2.1]nonane-9-carboxylate (400 mg, 46% yield).

Step 2: Into a 40 mL vial was added tert-butyl 2-[1-(4-chlorophenyl)cyclopropaneamido]-9-azabicyclo[4.2.1]nonane-9-carboxylate (400 mg, 0.955 mmol, 1 equiv), dichloromethane (DCM) (15 mL), and trifluoroacetic acid (TFA) (3 mL) at room temperature. The resulting mixture was stirred for 1 h at room temperature under nitrogen atmosphere. The resulting mixture was concentrated under reduced pressure. This resulted in N-{9-azabicyclo[4.2.1]nonan-2-yl}-1-(4-chlorophenyl)cyclopropane-1-carboxamide (300 mg, 99% yield), which was used in the next step directly without further purification.

Step 3: Into a 40 mL vial was added N-{9-azabicyclo[4.2.1]nonan-2-yl}-1-(4-chlorophenyl)cyclopropane-1-carboxamide (300 mg, 0.94 mmol, 1 equiv), BrCN (199.32 mg, 1.88 mmol, 2 equiv), K₂CO₃ (260.07 mg, 1.88 mmol, 2 equiv), and acetonitrile (MeCN) (10 mL) at room temperature.

The resulting mixture was stirred for 1 h at room temperature under nitrogen atmosphere. The reaction was monitored by LCMS. The reaction was quenched with water (10 ml) at room temperature. The resulting mixture was extracted with ethyl acetate (EtOAc) (3×40 mL), dried over anhydrous Na₂SO₄, filtered, and the filtrate concentrated under reduced pressure to provide 1-(4-chlorophenyl)-N-[9-cyano-9-azabicyclo[4.2.1]nonan-2-yl]cyclopropane-1-carboxamide (280 mg, 87% yield). The resulting mixture was used in the next step directly without further purification. LCMS (ESI): RT=0.950 min, m/z=344 [M+H]+.

Step 4: Into a 40 mL vial was added 1-(4-chlorophenyl)-N-[9-cyano-9-azabicyclo[4.2.1]nonan-2-yl]cyclopropane-1-carboxamide (200 mg, 0.582 mmol, 1 equiv), NH₄Cl (93.33 mg, 1.746 mmol, 3 equiv), trimethylsilyl azide (TMS-N₃) (407.57 mg, 1.746 mmol, 3 equiv), and dimethyl formamide (DMF) (10 mL) at room temperature. The resulting mixture was stirred for 2 h at 120° C. under nitrogen atmosphere.

The reaction was monitored by LCMS. The reaction was quenched by the addition of water (0.5 mL) at room temperature. The resulting mixture was concentrated under vacuum. The residue was purified by reversed-phase flash chromatography (C18 silica gel; mobile phase, acetonitrile (MeCN) in water, 40% to 50% gradient in 10 min; detector, UV 220 nm) to provide a crude product (250 mg), which was purified by PREP HPLC (Sunfire prep C18 column, 30*150 mm, 5 m; Mobile Phase A: acetonitrile, Mobile Phase B: Water (0.05% trifluoroacetic acid (TFA)); Flow rate: 60 mL/min; Gradient: 40% B to 50% B in 8 min, 50% B to 50% B in 9 min, 50% B; Wave Length: 254/220 nm; RT(min): 8.22) to provide N-(9-(1H-tetrazol-5-yl)-9-azabicyclo[4.2.1]nonan-2-yl)-1-(4-chlorophenyl)cyclopropane-1-carboxamide (Compound 9, rac-9) as a mixture of stereoisomers (150 mg, 47% yield). LCMS (ESI): RT(min)=88, m/z=387 [M+H]+.

Step 5: The mixture product rac-9 was purified by Prep-Chiral HPLC (CHIRALPAK IE, 2*25 cm, 5 m; Mobile Phase A: methyl tertbutyl ether (MBTE) (0.1% formic acid), Mobile Phase B: methanol:dichloromethane=1:1; Flow rate: 20 mL/min; Gradient: 20% B to 20% B in 25 min; Wave Length: 220/254 nm) to provide an assumed mixture of trans isomers Compounds 9B* and 9C* (RT(min): 10.72, 100 mg), assumed Compound 9A* (RT(min): 16.14, 6.7 mg) and assumed Compound 9D* (RT(min): 22.38, 6.2 mg). The assumed mixture of Compounds 9B* and 9C* was further purified by Prep-Chiral HPLC (CHIRAL ART Amylose-SA, 2*25 cm, 5 m; Mobile Phase A: hexanes (0.2% formic acid), Mobile Phase B: ethanol:dichloromethane=1:1; Flow rate: 20 mL/min; Gradient: 15% B to 15% B in 12 min; Wave Length: 220/254 nm) to provide Compound 9B* (RT(min): 7.32, 27.7 mg) and Compound 9C* (RT(min): 10.11, 33.1 mg). Stereochemistry was arbitrarily assigned for each isomer.

Compound 9A* LCMS (ESI): RT=1.529 min, m/z=387 [M+H]⁺; ¹H NMR (400 MHz, Methanol-d4) δ 7.43 (s, 4H), 4.32 (t, J=8.4 Hz, 1H), 4.25 (dd, J=8.5, 4.6 Hz, 1H), 4.09 (dt, J=9.4, 4.3 Hz, 1H), 2.43-2.28 (m, 2H), 2.01-1.86 (m, 1H), 1.76 (dd, J=10.4, 8.0 Hz, 2H), 1.68-1.47 (m, 6H), 1.13 (dtdd, J=12.7, 9.8, 6.1, 3.5 Hz, 3H).

Compound 9D* LCMS (ESI): RT=1.512 min, m/z=387 [M+H]⁺; ¹H NMR (400 MHz, Methanol-d4) δ 7.47-7.38 (m, 4H), 4.20 (dt, J=11.6, 5.5 Hz, 1H), 4.06-3.99 (m, 1H), 2.16-1.97 (m, 2H), 1.99-1.86 (m, 2H), 1.80 (ddd, J=18.4, 12.6, 5.4 Hz, 2H), 1.66 (d, J=8.8 Hz, 1H), 1.51 (dtdd, J=13.3, 9.5, 6.0, 3.4 Hz, 4H), 1.12 (dddd, J=31.5, 9.0, 5.9, 3.1 Hz, 2H).

Compound 9B* (27.7 mg, 18.47% yield). LCMS (ESI): RT=1.505 min, m/z=387 [M+H]⁺; ¹H NMR (400 MHz, Methanol-d4) δ 7.48-7.38 (m, 4H), 4.21 (dt, J=11.6, 5.6 Hz, 1H), 4.03 (d, J=6.6 Hz, 2H), 2.16-2.00 (m, 1H), 1.96 (dq, J=17.7, 6.8, 6.1 Hz, 3H), 1.79 (hept, J=6.5, 5.9 Hz, 2H), 1.64 (d, J=8.7 Hz, 1H), 1.51 (dtdd, J=13.3, 9.6, 6.1, 3.4 Hz, 4H), 1.16 (ddd, J=9.8, 6.1, 3.4 Hz, 1H), 1.08 (ddd, J=9.1, 6.1, 3.1 Hz, 1H).

Compound 9C* LCMS (ESI): RT=1.51 min, m/z=387 [M+H]+; 1H NMR (400 MHz, Methanol-d4) δ 7.47-7.38 (m, 4H), 4.20 (d, J=11.6 Hz, 1H), 4.06-3.99 (m, 1H), 2.16-1.97 (m, 2H), 1.99-1.86 (m, 2H), 1.80 (d, J=18.4 Hz, 2H), 1.66 (d, J=8.8 Hz, 1H), 1.51 (d, J=13.3 Hz, 4H), 1.12 (d, J=31.5 Hz, 2H).

### Example 10. Synthesis of (R)—N-(1-(1H-tetrazol-5-yl)piperidin-3-yl)-1-(4-chlorophenyl)cyclopropane-1-carboxamide (Compound 10A) and (S)—N-(1-(1H-tetrazol-5-yl)piperidin-3-yl)-1-(4-chlorophenyl)cyclopropane-1-carboxamide (Compound 10B)

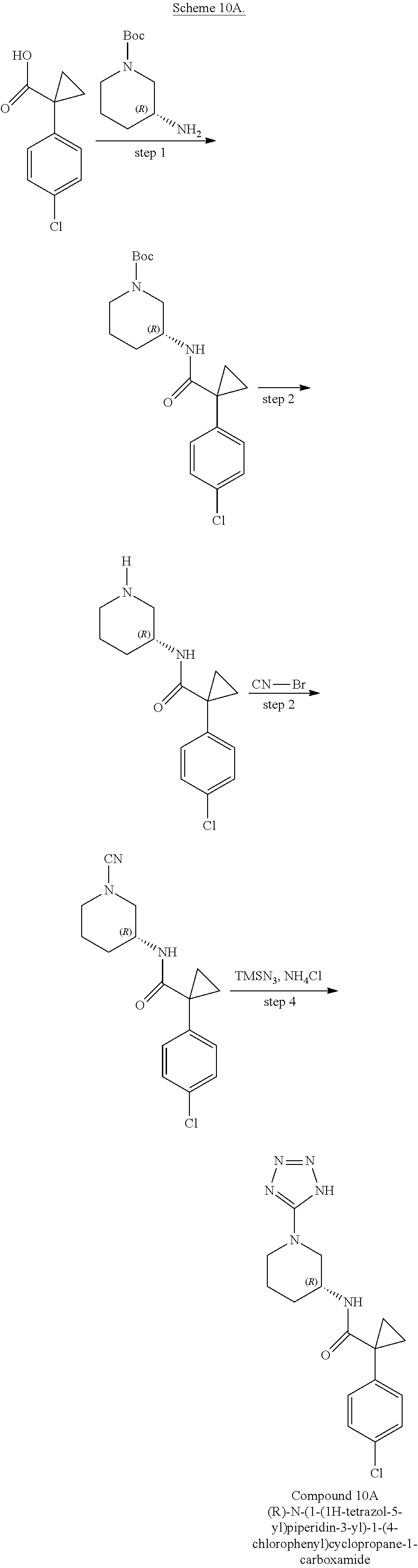

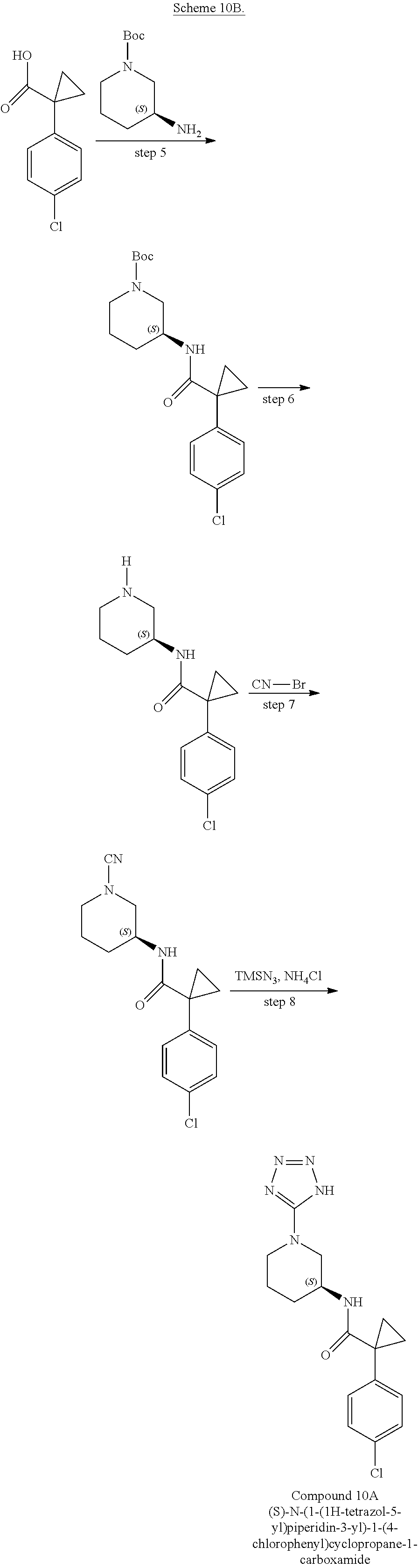

### Example 10 Follows Protocol C

Step 1: Into an 8 mL vial was added tert-butyl (R)-3-aminopiperidine-1-carboxylate (“amine (iv) reagent”) (565 mg, 2.82 mmol, 1 equiv), 1-(4-chlorophenyl)cyclopropane-1-carboxylic acid (“carboxyl (ii) reagent”) (782.22 mg, 3.38 mmol, 1.2 equiv), dichloromethane (DCM) (5 mL, 78.65 mmol), and triethylamine (TEA) (856.38 mg, 8.46 mmol, 3 equiv). The resulting mixture was stirred for 2 h at 30° C. The reaction was monitored by LCMS. The reaction was then quenched by the addition of 20 mL of water. The resulting solution was extracted with ethyl acetate (3×20 mL). The residue purified by silica gel column with ethyl acetate/petroleum ether (2:3) as elutant. This resulted in tert-butyl (R)-3-(1-(4-chlorophenyl)cyclopropane-1-carboxamido)piperidine-1-carboxylate (400 mg, 37% yield). LCMS (ES, m/z): RT=1.04 min, m/z=379.0[M+1]⁺.

Step 2: Into a 50-mL round-bottom flask, was placed tert-butyl 3-[1-(4-chlorophenyl)cyclopropaneamido]piperidine-1-carboxylate (400 mg, 1.05 mmol, 1 equiv), and HCl (gas) in 1,4-dioxane (4 mL, 131.64 mmol). The resulting solution was stirred for 2 h at 25° C., then concentrated under vacuum. Desired product could be detected by LCMS. This resulted in (R)-1-(4-chlorophenyl)-N-(piperidin-3-yl)cyclopropane-1-carboxamide (290 mg, 99% yield). LCMS (ES, m/z): RT=0.57 min, m/z=279.0[M+1]⁺.

Step 3: Into a 20-mL vial, was placed 1-(4-chlorophenyl)-N-(piperidin-3-yl)cyclopropane-1-carboxamide (300 mg, 1.076 mmol, 1 equiv), cyanogen bromide (136.78 mg, 1.291 mmol, 1.2 equiv), triethylamine (TEA) (326.68 mg, 3.228 mmol, 3 equiv), and dichloromethane (DCM) (3 mL, 47.190 mmol). The resulting solution was stirred for 2 h at 25° C. The desired product could be detected by LCMS. The resulting mixture was concentrated under vacuum. The residue was applied onto a silica gel column with ethyl acetate/petroleum ether (2:3) as elutant. This resulted in (R)-1-(4-chlorophenyl)-N-(1-cyanopiperidin-3-yl)cyclopropane-1-carboxamide (200 mg, 61% yield). LCMS (ES, m/z): RT=0.99 min, m/z=304.0[M+1]⁺.

Step 4: Into an 8-mL microwave tube purged and maintained with an inert atmosphere of nitrogen, was placed (R)-1-(4-chlorophenyl)-N-(1-cyanopiperidin-3-yl)cyclopropane-1-carboxamide (180 mg, 0.59 mmol, 1 equiv), azidotrimethylsilane (81.92 mg, 0.71 mmol, 1.2 equiv), NH₄Cl (95.08 mg, 1.77 mmol, 3 equiv), and dimethyl formamide (DMF) (2 mL, 25.84 mmol, 43.62 equiv). The resulting solution was stirred for 2 h at 120° C. in an oil bath. The desired product could be detected by LCMS. The residue was purified by reverse flash chromatography (C18 silica gel; mobile phase, triethylamine (TEA) in water, 10% to 50% gradient in 10 min; detector, UV 254 nm) to provide (R)—N-(1-(1H-tetrazol-5-yl)piperidin-3-yl)-1-(4-chlorophenyl)cyclopropane-1-carboxamide (Compound 10A) (150 mg). Compound 10A was further purified by chiral-PREP HPLC (CHIRAL ART Cellulose-SB, 2*25 cm, 5 m; Mobile Phase A: Hexanes (0.1% formic acid), Mobile Phase B: ethanol (EtOH); Flow rate: 20 mL/min; Gradient: 10% B to 10% B in 16 min; Wave Length: 220/254 nm) to provide Compound 10A (21.9 mg, 11% yield). LCMS (ES, m/z): RT=0.66 min, m/z=347.0[M+1]⁺. ¹H NMR (400 MHz, DMSO-d₆) δ 14.86 (s, 1H), 7.61-7.17 (m, 4H), 6.84 (d, J=8.0 Hz, 1H), 3.89-3.72 (m, 1H), 3.70-3.52 (m, 2H), 3.00-2.81 (m, 2H), 1.68 (t, J1=J2=14.0 Hz, 2H), 1.57-1.39 (m, 2H), 1.38-1.28 (m, 2H), 1.10-0.82 (m, 2H).

Steps 5-8: Compound 10B was synthesized following Example 10 and Compound 10A synthesis, but using tert-butyl (S)-3-aminopiperidine-1-carboxylate as the starting material. LCMS (ES, m/z): RT=0.66 min, m/z=347.0[M+1]⁺. ¹H NMR (400 MHz, DMSO-d₆) δ14.89 (s, 1H), 7.41-7.36 (m, 2H), 7.35-7.30 (m, 2H), 6.82 (d, J=8.2 Hz, 1H), 3.85-3.73 (m, 1H), 3.68-3.57 (m, 2H), 2.90 (dt, J=12.2, 9.2 Hz, 2H), 1.76-1.60 (m, 2H), 1.54-1.40 (m, 2H), 1.35 (m, 2H), 0.99 (q, J=3.8 Hz, 2H).

### Example 11. Synthesis of 1-(4-chlorophenyl)-N-(3-methyl-1-(1H-tetrazol-5-yl)piperidin-3-yl)cyclopropane-1-carboxamide (Compound 11, rac-11), and Compounds 11A* and 11B*

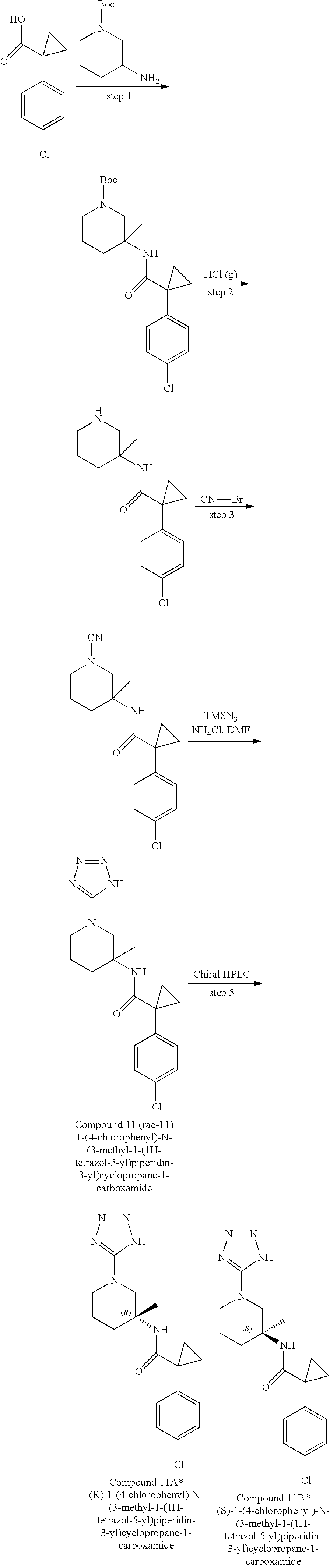

### Example 11 Follows Protocol C

Step 1: Into a 20-mL vial, was placed 1-(4-chlorophenyl)cyclopropane-1-carboxylic acid (“carboxyl (ii) reagent”) (330 mg, 1.67 mmol, 1 equiv), dichloromethane (DCM) (5 mL) and SOCl₂ (1.80 g, 16.70 mmol, 10 equiv). The resulting solution was stirred for 2 h at room temperature. The reaction progress was monitored by LCMS, The resulting solution was concentrated under vacuum, then the residue was dissolved in DCM (2 mL) was dropwise into a solution of tert-butyl 3-amino-3-methylpiperidine-1-carboxylate (“amine (iv) reagent”) (280 mg, 1.30 mmol, 1 equiv), triethylamine (528 mg, 5.22 mmol, 4 equiv) in DCM (2.5 mL) at 0° C., the reaction was slowly to warm to room temperature for 1 h, the reaction progress was monitored by LCMS. The reaction was quenched with 5 ml of H₂O, extracted with DCM (2×30 mL), dried over anhydrous Na₂SO₄. After filtration, the filtrate was concentrated under reduced pressure. The residue was applied onto a silica gel column with ethyl acetate/petroleum ether (2:3). This resulted in tert-butyl 3-[1-(4-chlorophenyl)cyclopropaneamido]-3-methylpiperidine-1-carboxylate (380 mg, 74% yield). LCMS (ES, m/z): RT=1.17 min, m/z=393.0[M+1]+.

Step 2: Into a 25 mL round bottom flask was added tert-butyl 3-[1-(4-chlorophenyl)cyclopropaneamido]-3-methylpiperidine-1-carboxylate (320 mg, 0.81 mmol, 1 equiv) and HCl(gas) in 1,4-dioxane (4M, 3 mL) at room temperature. The resulting solution was stirred for 3 h at room temperature. The reaction progress was monitored by LCMS. The resulting solution was concentrated under vacuum. This resulted in 1-(4-chlorophenyl)-N-(3-methylpiperidin-3-yl)cyclopropane-1-carboxamide HCl salt (238 mg). LCMS (ES, m/z): RT=0.70 min, m/z=293.0[M+1]⁺.

Step 3: Into a 20-mL vial was added 1-(4-chlorophenyl)-N-(3-methylpiperidin-3-yl)cyclopropane-1-carboxamide HCl salt (240.00 mg, 0.82 mmol, 1 equiv), cyanogen bromide (521 mg, 4.92 mmol, 6 equiv), acetonitrile (3 mL) and potassium carbonate (678.96 mg, 4.92 mmol, 6 equiv) at room temperature. The resulting solution was stirred for 16 h at 80° C. The reaction progress was monitored by LCMS. The reaction was quenched with 5.00 ml of H₂O, extracted with DCM (2×30 ml), dried over anhydrous Na₂SO₄. After filtration, the filtrate was concentrated under reduced pressure. The residue was applied onto a silica gel column with ethyl acetate/petroleum ether (2:3). This resulted in 1-(4-chlorophenyl)-N-(1-cyano-3-methylpiperidin-3-yl)cyclopropane-1-carboxamide (250 mg, 96% yield).

Step 4: Into a 10-mL microwave tube was added 1-(4-chlorophenyl)-N-(1-cyano-3-methylpiperidin-3-yl)cyclopropane-1-carboxamide (220 mg, 0.69 mmol, 1 equiv), trimethylsilyl azide (239 mg, 2.07 mmol, 3 equiv) NH₄Cl (111 mg, 2.07 mmol, 3 equiv), and dimethyl formamide (DMF) (2.5 mL). The resulting solution was stirred for 2.5 h at 120° C. for 2h. the reaction progress was monitored by LCMS. The reaction was quenched with 5.00 mL of H₂O, extracted with DCM (2×30 ml), dried over anhydrous Na₂SO₄. After filtration, the filtrate was concentrated under reduced pressure. The residue was purified by reverse flash chromatography (C18 silica gel; mobile phase, acetonitrile in H₂O, 0% to 100% gradient in 30 min; detector, UV 220/254 nm) to provide 1-(4-chlorophenyl)-N-(3-methyl-1-(1H-tetrazol-5-yl)piperidin-3-yl)cyclopropanecarboxamide (Compound 11, rac-11) (100 mg, 40% yield, 95% purity).

Step 5: The two stereoisomers of the product, rac-11, (99 mg, 95% purity) were separated by chiral-PREP HPLC (CHIRAL ART Cellulose-SC, 2*25 cm, 5 μm; Mobile Phase A: Hexanes (0.2% formic acid), Mobile Phase B: Methanol (MeOH): ethanol (EtOH)=1:1; Flow rate: 20 mL/min; Gradient: 20% B to 20% B in 14 min; Wave Length: 220/254 nm) to provide Compound 11B* (RT(min): 13.27, 23.9 mg, 12% yield) and Compound 11A* (RT(min): 10.89, 31.9 mg, 16% yield). Stereochemistry was arbitrarily assigned.

Compound 11A*: LCMS (ES, m/z): RT=0.87 min, m/z=361.0[M+1]+. H NMR (400 MHz, DMSO-d₆) δ 7.38-7.09 (m, 4H), 5.80 (s, 1H), 3.85 (d, J=12.8 Hz, 1H), 3.61 (d, J=12.4 Hz, 1H), 3.08-2.89 (m, 2H), 2.06 (d, J=10.4 Hz, 1H), 1.54-1.31 (m, 3H), 1.25 (m, 5H), 0.96-0.83 (m, 2H).

Compound 11B*: LCMS (ES, m/z): RT=0.90 min, m/z=361.0[M+1]+. H NMR (400 MHz, DMSO-d₆) δ 7.41-7.06 (m, 4H), 5.81 (s, 1H), 3.84 (d, J=12.8 Hz, 1H), 3.61 (d, J=12.4 Hz, 1H), 3.08-2.85 (m, 2H), 2.06 (d, J=11.2 Hz, 1H), 1.56-1.30 (m, 3H), 1.30-1.18 (m, 5H), 0.97-0.82 (m, 2H).

### Example 12. Synthesis of 1-(4-chlorophenyl)-N-(5-methyl-1-(1H-tetrazol-5-yl)azepan-3-yl)cyclopropane-1-carboxamide (Compound 12, rac-12) and Compounds 12A*, 12B*, 12C*, and 12D*

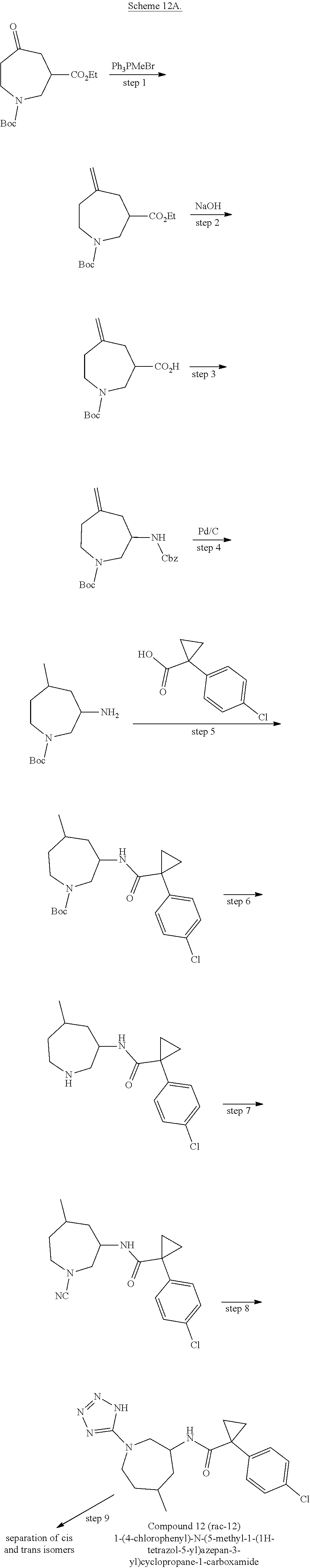

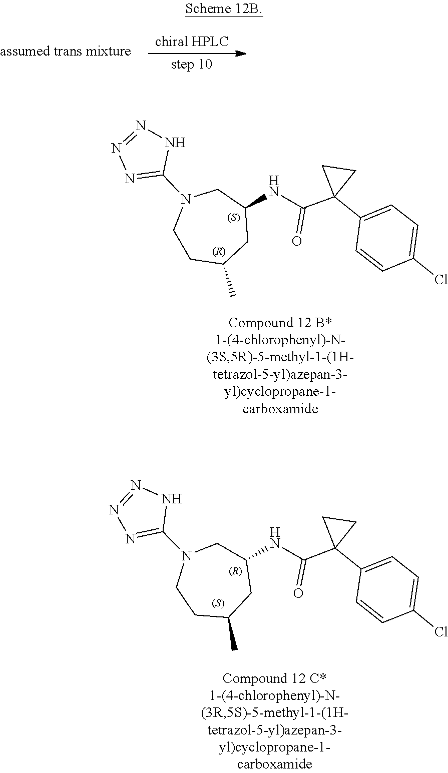

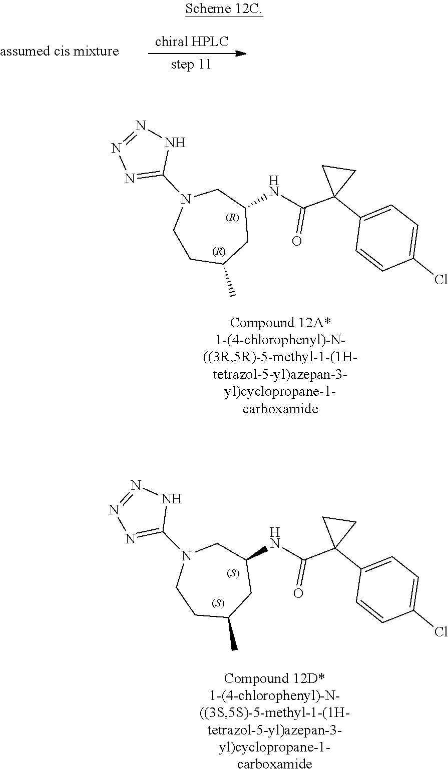

### Example 12 Follows Protocol C

Step 1: In a 500-mL round bottom flask, to a solution of bromo(methyl)triphenyl-lambda5-phosphane (2.75 g, 7.71 mmol, 1.1 equiv) in tetrahydrofuran (THF) (200 mL) was added n-butyl lithium (n-BuLi) (3.08 mL, 7.71 mmol, 1.1 equiv) at 0° C. under N₂ atmosphere. The reaction mixture was stirred at 0° C. for 30 min. Then a solution of 1-tert-butyl 3-ethyl 5-oxoazepane-1,3-dicarboxylate (2.00 g, 7.00 mmol, 1 equiv) in tetrahydrofuran (THF) was added and the mixture was stirred for another 60 mins at 0° C. The reaction progress was monitored by LCMS. The reaction was quenched with 100 mL NH₄Cl (aq). The resulting mixture was extracted with ethyl acetate (EtOAc) (3×500 mL) and dried over anhydrous Na₂SO₄. After filtration, the filtrate was concentrated under reduced pressure. The residue was purified by flash chromatography (silica gel; mobile phase, ethyl acetate in petroleum ether, 0% to 100% gradient in 40 min; detector, UV 254 nm) to provide 1-tert-butyl 3-ethyl 5-methylideneazepane-1,3-dicarboxylate (1.00 g, 50% yield). LCMS (ES, m/z): RT=1.050 min, m/z=284 [M+1]⁺.

Step 2: Into a 100 mL round-bottom flask was added 1-tert-butyl 3-ethyl 5-methylideneazepane-1,3-dicarboxylate (1 g, 3.52 mmol, 1 equiv), NaOH (282 mg, 7.05 mmol, 2 equiv), methanol (MeOH) (50 mL), and water (10 mL) at room temperature. The resulting mixture was stirred for 1 h at room temperature. The reaction progress was monitored by LCMS. The resulting mixture was quenched with 100 mL of water, extracted with ethyl acetate (EtOAc) (2×200 mL). The aqueous layer was acidified to pH 6 with HCl (1M). The resulting mixture was extracted with EtOAc (3×200 mL). The combined organic layers were dried over anhydrous Na₂SO₄. After filtration, the filtrate was concentrated under reduced pressure to provide 5-methylideneazepane-1,3-dicarboxylic acid (500 mg, crude), which was used in the next step without purification. LCMS (ES, m/z): RT=0.805 min, m/z=256 [M+1]⁺.

Step 3: Into a 40 mL vial was added 1-(tert-butoxycarbonyl)-5-methylideneazepane-3-carboxylic acid (500 mg, 1.95 mmol, 1 equiv), benzyl alcohol (2 mL, 18.49 mmol, 9.44 equiv), diphenylphosphoryl azide (DPPA) (1077.90 mg, 3.91 mmol, 2 equiv), triethylamine (TEA) (990.87 mg, 9.79 mmol, 5 equiv), and toluene (20 mL) at room temperature. The resulting mixture was stirred for 2 h at 100° C. under N₂. The reaction progress was monitored by LCMS. The resulting mixture was concentrated under reduced pressure. The residue was purified by reverse flash chromatography (C18 silica gel; mobile phase, acetonitrile (MeCN) in water, 0% to 50% gradient in 30 min; detector, UV 254 nm) to provide tert-butyl 3-{[(benzyloxy)carbonyl]amino}-5-methylideneazepane-1-carboxylate (400 mg, 57% yield). LCMS (ES, m/z): RT=1.061 min, m/z=361 [M+1]⁺.

Step 4: Into a 25 mL vial was added tert-butyl 3-{[(benzyloxy)carbonyl]amino}-5-methylideneazepane-1-carboxylate (400 mg, 1.11 mmol, 1 equiv), Pd/C (100 mg), and methanol (MeOH) (15 mL) at room temperature. The resulting mixture was stirred for 1 h at room temperature under hydrogen atmosphere. The reaction was monitored by LCMS. The resulting mixture was filtered, the filter cake was washed with methanol (MeOH) (3×10 mL). The filtrate was concentrated under reduced pressure to provide tert-butyl 3-amino-5-methylazepane-1-carboxylate (250 mg, 99% yield). LCMS (ES, m/z): RT=0.596 min, m/z=229 [M+1]⁺.

Step 5: Into an 8 mL vial was added tert-butyl 3-amino-5-methylazepane-1-carboxylate (“amine (iv) reagent”) (250 mg, 1.09 mmol, 1 equiv), 1-(4-chlorophenyl)cyclopropane-1-carboxylic acid (“carboxyl (ii) reagent”) (258.34 mg, 1.31 mmol, 1.2 equiv), N-methyl imidazole (NMI) (269.69 mg, 3.28 mmol, 3 equiv), N,N,N′,N′-tetramethylchloroformaridinium hexafluorophosphate (TCFH) (399.36 mg, 1.42 mmol, 1.3 equiv), and acetonitrile (MeCN) (5 mL) at room temperature. The resulting mixture was stirred for 2 h at room temperature. The reaction progress was monitored by LCMS. The residue was purified by reverse flash chromatography (C18 silica gel; mobile phase, acetonitrile (MeCN) in water, 0% to 100% gradient in 40 min; detector, UV 254 nm) to provide tert-butyl 3-[1-(4-chlorophenyl)cyclopropaneamido]-5-methylazepane-1-carboxylate (200 mg, 45% yield). LCMS (ES, m/z): RT=1.176 min, m/z=407 [M+1]⁺.

Step 6: Into an 8 mL vial was added tert-butyl 3-[1-(4-chlorophenyl)cyclopropaneamido]-5-methylazepane-1-carboxylate (180 mg, 0.44 mmol, 1 equiv) and HCl (gas) in 1,4-dioxane (2 mL) at room temperature. The resulting mixture was stirred for 1 h at room temperature. The reaction progress was monitored by LCMS. The resulting mixture was concentrated under vacuum to provide 1-(4-chlorophenyl)-N-(5-methylazepan-3-yl)cyclopropane-1-carboxamide (130 mg, HCl salt). LCMS (ES, m/z): RT=0.687 min, m/z=307 [M+1]⁺.

Step 7: Into an 8 mL vial was added 1-(4-chlorophenyl)-N-(5-methylazepan-3-yl)cyclopropane-1-carboxamide (120 mg, 0.39 mmol, 1 equiv), BrCN (82.85 mg, 0.78 mmol, 2 equiv), and acetonitrile (MeCN) (3 mL) at room temperature. The resulting mixture was stirred for 1 h at room temperature. The reaction progress was monitored by LCMS. The residue was purified by reverse flash chromatography (C18 silica gel; mobile phase, acetonitrile (MeCN) in water, 0% to 100% gradient in 30 min; detector, UV 254 nm) to provide 1-(4-chlorophenyl)-N-(1-cyano-5-methylazepan-3-yl)cyclopropane-1-carboxamide (120 mg, 92% yield). LCMS (ES, m/z): RT=0.923 min, m/z=332 [M+1]⁺.

Step 8: Into an 8 mL vial was added 1-(4-chlorophenyl)-N-(1-cyano-5-methylazepan-3-yl)cyclopropane-1-carboxamide (80 mg, 0.24 mmol, 1 equiv), trimethylsilyl azide (55.6 mg, 0.48 mmol, 2 equiv), dimethyl formamide (DMF) (3 mL), and NH₄Cl (51.6 mg, 0.96 mmol, 4 equiv) at room temperature. The resulting mixture was stirred for 2 h at 120° C. The reaction progress was monitored by LCMS. The reaction was quenched with 1 mL water. The resulting mixture was filtered and concentrated to provide the crude product, 1-(4-chlorophenyl)-N-(5-methyl-1-(1H-tetrazol-5-yl)azepan-3-yl)cyclopropane-1-carboxamide (Compound 12, rac-12), as a mixture of cis and trans stereoisomers.

Step 8: The crude product, rac-12, was purified by PREP HPLC (XBridge Shield RP18 OBD Column, 30*150 mm, 5 μm; mobile phase, water (10 mmol/L NH₄HCO₃) and acetonitrile (MeCN) (25% acetonitrile (MeCN) up to 40% in 8 min); Detector, UV 254 nm) to provide an assumed mixture of cis isomers as first eluting peak, and assumed trans isomers as second eluting peak. Stereochemistry arbitrarily assigned.

Step 9: The assumed trans isomeric mixture of rac-12 was purified by Chiral-PREP HPLC (CHIRALPAK IG, 2*25 cm, 5 μm; Mobile Phase A: Hexanes (0.2% formic acid), Mobile Phase B: ethanol:dichloromethane=1:1; Flow rate: 20 mL/min; Gradient: 15% B to 15% B in 22.2 min; Wave Length: 220/254 nm) to provide Compound 12B* (RT(min): 16.3, 2.6 mg) and Compound 12C* (RT(min): 19.7, 5.1 mg). Stereochemistry was arbitrarily assigned.

Compound 12B*: LCMS (ES, m/z): RT=1.432 min, m/z=375 [M+1]⁺. ¹H NMR (400 MHz, Methanol-d₄) δ 7.40 (s, 4H), 4.05-4.93 (m, 1H), 3.66-3.54 (m, 2H), 3.53-3.40 (m, 2H), 1.89-1.79 (m, 1H), 1.74-1.59 (m, 2H), 1.58-1.52 (m, 2H), 1.31 (s, 1H), 1.14-1.06 (m, 2H), 0.97 (d, J=6.7 Hz, 3H), 0.95-0.88 (m, 1H).

Compound 12C*: LCMS (ES, m/z): RT=1.101 min, m/z=375 [M+1]⁺. ¹H NMR (400 MHz, Methanol-d₄) δ 7.26-7.14 (m, 4H), 4.24-4.17 (m, 1H), 3.88-3.78 (m, 1H), 3.59-3.52 (m, 1H), 3.48-3.39 (m, 1H), 3.18-3.07 (m, 1H), 1.83-1.71 (m, 2H), 1.71-1.60 (m, 1H), 1.58-1.45 (m, 3H), 1.43-1.37 (m, 1H), 1.12-1.02 (m, 2H), 0.98 (d, J=6.7 Hz, 3H).

Step 10: The assumed cis isomeric mixture of rac-12 was purified by Chiral-PREP HPLC (CHIRAL ART Cellulose-SB, 2*25 cm, 5 μm; Mobile Phase A: Hexanes (0.2% formic acid), Mobile Phase B: isopropanol; Flow rate: 20 mL/min; Gradient: 10% B to 10% B in 24 min; Wave Length: 220/254 nm) to provide Compound 12A* (RT(min): 18.03, 2.60 mg) and Compound 12D* (RT(min): 21.84, 5.1 mg). Stereochemistry was arbitrarily assigned.

Compound 12A*: LCMS (ES, m/z): RT=1.551 min, m/z=375 [M+1]⁺. ¹H NMR (400 MHz, Methanol-d₄) δ 7.40 (s, 4H), 4.05-4.93 (m, 1H), 3.66-3.53 (m, 2H), 3.53-3.40 (m, 2H), 1.89-1.80 (m, 1H), 1.74-1.60 (m, 2H), 1.59-1.43 (m, 3H), 1.22-1.16 (m, 1H), 1.16-1.06 (m, 2H), 0.97 (d, J=6.7 Hz, 3H).

Compound 12D*: LCMS (ES, m/z): RT=0.802 min, m/z=375 [M+1]⁺. ¹H NMR (400 MHz, Methanol-d₄) δ 7.28-7.13 (m, 4H), 4.20 (q, J=5.0, 3.7 Hz, 1H), 3.89-3.78 (m, 1H), 3.61-3.52 (m, 1H), 3.49-3.40 (m, 1H), 3.18-3.06 (m, 1H), 1.83-1.71 (m, 2H), 1.70-1.60 (m, 1H), 1.58-1.45 (m, 3H), 1.43-1.35 (m, 1H), 1.11-1.02 (m, 2H), 0.98 (d, J=6.6 Hz, 3H).

### Example 13. Synthesis of 1-(4-chlorophenyl)-N-(5-cyclopropyl-1-(1H-tetrazol-5-yl)piperidin-3-yl)cyclopropane-1-carboxamide (Compound 13, rac-13), and Compounds 13A*, 13B*, 13C*, and 13D*

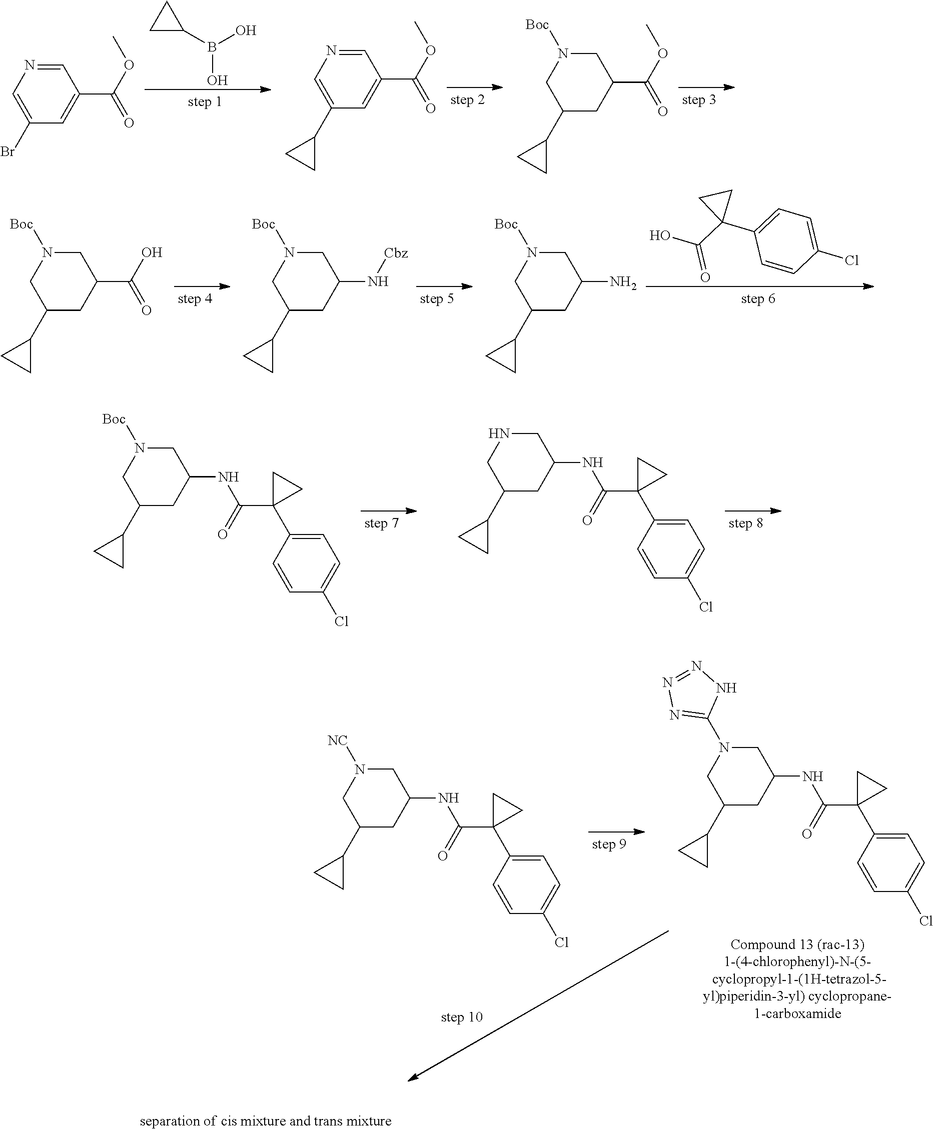

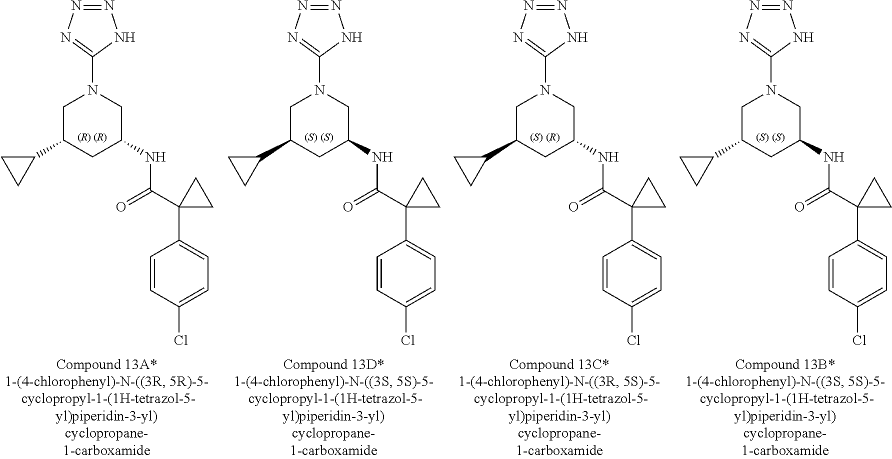

### Example 13 Follows Protocol C

Step 1: To a stirred solution of methyl 5-bromopyridine-3-carboxylate (1 g, 4.629 mmol, 1 equiv) and cyclopropylboronic acid (2.39 g, 27.774 mmol, 6 equiv) in dioxane (10 mL) and water (1 mL) was added [1,1-Bis(diphenylphosphino)ferrocene]palladiun(II) dichloride (Pd(dppf)Cl₂), dichloromethane (DCM) (1.35 g, 1.852 mmol, 0.4 equiv) and Na₂CO₃ (0.98 g, 9.258 mmol, 2 equiv) at room temperature.

The resulting mixture was stirred overnight at 80° C. under nitrogen atmosphere. The resulting mixture was poured into water and extracted with ethyl acetate (EtOAc) (3×10 mL). The combined organic layers were washed with water (3×10 mL) and brine (3×10 mL), dried over anhydrous Na₂SO₄. After filtration, the filtrate was concentrated under reduced pressure. The residue was purified by silica gel column chromatography, eluting with petroleum ether:ethyl acetate (56:44) to provide methyl 5-cyclopropylpyridine-3-carboxylate (0.68 g, 83% yield). LCMS (ES, m/z): RT=0.71 min, m/z=178.2[M+1]⁺.

Step 2: To a stirred solution of methyl 5-cyclopropylpyridine-3-carboxylate (300 mg, 1.693 mmol, 1 equiv) in methanol (MeOH) (1.5 mL) and acetic acid (AcOH) (1.5 mL) was added PtO₂ (192.22 mg, 0.847 mmol, 0.5 equiv) in portions at room temperature. The resulting mixture was stirred overnight at 50° C. under hydrogen atmosphere. The resulting mixture was filtered, the filter cake was washed with methanol (MeOH) (3×10 mL). The filtrate was concentrated under reduced pressure. To the above mixture was added ditertbutyldicarbonate (Boc₂O) (2.86 g, 13.10 mmol, 1.20 equiv) by dropwise at room temperature. The resulting mixture was stirred for additional 1 h at room temperature. The reaction was monitored by LCMS. The resulting mixture was quenched with water (10 mL) and extracted with EtOAc (3×30 mL). The combined organic layers were washed with brine (30 mL), dried over anhydrous Na₂SO₄. After filtration, the filtrate was concentrated under reduced pressure. The residue was purified by silica gel column chromatography, eluted with (dichloromethane/petroleum ether=6:4) to provide 1-tert-butyl 3-methyl 5-cyclopropylpiperidine-1,3-dicarboxylate (800 mg, 42% yield). LCMS (ES, m/z): RT=0.66 min, m/z=284.2[M+H]⁺.

Step 3: Into a 40 mL vial was added 1-(tert-butyl) 3-methyl 5-cyclopropylpiperidine-1,3-dicarboxylate (600 mg, 2.12 mmol, 1 equiv), methanol (MeOH) (8 mL), water (2 mL) and LiOH (152.12 mg, 6.35 mmol, 3 equiv) at room temperature. The resulting mixture was stirred overnight at room temperature, then concentrated under vacuum. The aqueous layer was extracted with ethyl acetate (EtOAc) (3×30 mL). The resulting mixture was concentrated under vacuum. This resulted in (tert-butoxycarbonyl)-5-cyclopropylpiperidine-3-carboxylic acid (506 mg, 89% yield). LCMS (ES, m/z): RT=0.85 min, m/z=268.0[M−1]⁻.

Step 4: Into an 8 mL vial was added (tert-butoxycarbonyl)-5-cyclopropylpiperidine-3-carboxylic acid (400 mg, 1.49 mmol, 1 equiv), toluene (0.40 mL), benzyl alcohol (4 mL), diphenylphosphoryl azide (DPPA) (1226.10 mg, 4.46 mmol, 3 equiv) and triethylamine (TEA) (450.83 mg, 4.46 mmol, 3 equiv) under nitrogen atmosphere at room temperature. The resulting mixture was stirred for 2 h at 100° C. The reaction was quenched with water at room temperature. The resulting mixture was concentrated under vacuum. The aqueous layer was extracted with EtOAc (3×20 mL). The resulting mixture was concentrated under vacuum. The residue was purified by silica gel column chromatography, eluting with dichloromethane (DCM):petroleum ether (PE) (5:4) to provide tert-butyl 3-(((benzyloxy)carbonyl)amino)-5-cyclopropylpiperidine-1-carboxylate (140 mg, 25% yield). LCMS (ES, m/z): RT=1.10 min, m/z=375.0[M−1]⁻.

Step 5: To a solution of tert-butyl 3-(((benzyloxy)carbonyl)amino)-5-cyclopropylpiperidine-1-carboxylate (300 mg, 0.80 mmol, 1 equiv) in methanol (MeOH) (5 mL) was added Pd/C (10%, 225.76 mg) in a pressure tank. The mixture was hydrogenated at room temperature under 10 psi of hydrogen pressure for 2 h, filtered through a Celite pad and concentrated under reduced pressure. This resulted in tert-butyl 3-amino-5-cyclopropylpiperidine-1-carboxylate (180 mg, 98.40% yield). LCMS (ES, m/z): RT=0.87 min, m/z=241.0[M+1]⁺.

Step 6: Into an 8 mL vial was added tert-butyl 3-amino-5-cyclopropylpiperidine-1-carboxylate (“amine (iv) reagent”) (100 mg, 0.416 mmol, 1 equiv), acetonitrile (MeCN) (1 mL), 1-(4-chlorophenyl)cyclopropane-1-carboxylic acid (“carboxyl (ii) reagent”) (81.81 mg, 0.416 mmol, 1 equiv), N,N,N,N-tetrametivlchliorofoninanidinium hexatluorophosphate (TCFH) (151.76 mg, 0.541 mmol, 1.30 equiv) and N-methyl imidazole (NMI) (102.48 mg, 1.248 mmol, 3 equiv) at room temperature. The resulting mixture was stirred for 2 h at room temperature. The resulting mixture was concentrated under vacuum. The residue was purified by silica gel column chromatography, eluting with dichloromethane (DCM):methanol (MeOH) (4:1) to provide tert-butyl 3-(1-(4-chlorophenyl)cyclopropane-1-carboxamido)-5-cyclopropylpiperidine-1-carboxylate (80 mg, 45.8% yield). LCMS (ES, m/z): RT=1.19 min, m/z=419.1[M+1]⁺.

Step 7: A mixture of tert-butyl 3-(1-(4-chlorophenyl)cyclopropane-1-carboxamido)-5-cyclopropylpiperidine-1-carboxylate (70 mg, 0.024 mmol, 1 equiv) and HCl (gas) in 1,4-dioxane (1 mL) was stirred for 2 h at room temperature. The reaction was monitored by LCMS, then concentrated under reduced pressure. This resulted in 1-(4-chlorophenyl)-N-(5-cyclopropylpiperidin-3-yl)cyclopropane-1-carboxamide (50 mg, 96% yield). LCMS (ES, m/z): RT=1.07 min, m/z=319.1[M−1]⁻.

Step 8: A mixture of 1-(4-chlorophenyl)-N-(5-cyclopropylpiperidin-3-yl)cyclopropane-1-carboxamide (50 mg, 0.157 mmol, 1 equiv) and cyanogen bromide (49.83 mg, 0.471 mmol, 3 equiv), triethylamine (TEA) (47.61 mg, 0.471 mmol, 3 equiv), and acetonitrile (MeCN) (2 mL) was stirred for 2 h at 80° C. The reaction was monitored by LCMS. The resulting mixture was diluted with water (10 mL).

The aqueous layer was extracted with EtOAc (3×10 mL), organic layers combined, dried, and concentrated under reduced pressure. The residue was purified by silica gel column chromatography, eluting with petroleum ether:ethyl acetate (1:1) to provide 1-(4-chlorophenyl)-N-(1-cyano-5-cyclopropylpiperidin-3-yl)cyclopropane-1-carboxamide (35 mg, 65% yield). LCMS (ES, m/z): RT=1.14 min, m/z=344.1[M+1]⁺.

Step 9: A mixture of 1-(4-chlorophenyl)-N-(1-cyano-5-cyclopropylpiperidin-3-yl)cyclopropane-1-carboxamide (50 mg, 0.145 mmol, 1 equiv), trimethylsilyl azide (50.26 mg, 0.435 mmol, 3 equiv), NH₄Cl (23.33 mg, 0.435 mmol, 3 equiv) in dimethylformamide (DMF) (1 mL) was stirred for 2 h at 120° C. The reaction was monitored by LCMS. The resulting mixture was filtered, the filter cake was washed with methanol (MeOH) (3×4 mL), and the filtrate was concentrated under reduced pressure to provide a residue, which was purified by reverse flash chromatography (silica gel; mobile phase, acetonitrile (MeCN) in water, 50% to 70% gradient in 10 min; detector, UV 254 nm) to provide crude 1-(4-chlorophenyl)-N-(5-cyclopropyl-1-(1H-tetrazol-5-yl)piperidin-3-yl)cyclopropane-1-carboxamide (Compound 13, rac-13) (30 mg) as a mixture of cis and trans isomers.

Step 10. The crude product, rac-13, was purified by PREP HPLC (Xselect CSH C18 OBD Column 30*150 mm 5 m, n; Mobile Phase A: water(10 mmol/L NH₄HCO₃), Mobile Phase B: acetonitrile (MeCN); Flow rate: 60 mL/min; Gradient: 24% B to 34% B in 8 min, 34% B; Wave Length: 254 nm; to provide an assumed mixture of cis isomers (Compounds 13A* and 13D*) as a first eluting peak and an assumed mixture of trans isomers (Compounds 13B* and 13C*) as a second eluting peak, stereochemistry arbitrarily assigned.

Mixture of Compounds 13A* and 13D*: LCMS (ES, m/z): RT=0.72 min, m/z=387.1[M+H]⁺. ¹H NMR (400 MHz, DMSO-d₆) δ 7.33-7.24 (m, 4H), 6.58 (d, J=7.7 Hz, 1H), 4.03 (s, 1H), 3.24-3.21 (m, 2H), 3.20-3.16 (m, 2H), 3.00-2.90 (m, 1H), 1.73-1.63 (m, 1H), 1.53-1.42 (m, 1H), 1.37-1.22 (m, 2H), 0.97 (td, J=8.3, 6.9, 4.6 Hz, 2H), 0.80-0.70 (m, 1H), 0.64 (m, J=8.4, 4.8 Hz, 1H), 0.47-0.33 (m, 2H), 0.07 (d, J=4.9 Hz, 1H).

Mixture of Compounds 13B* and 13C*: LCMS (ES, m/z): RT=0.81 min, m/z=387.1[M+H]⁺. ¹H NMR (400 MHz, DMSO-d₆) δ 7.45-7.37 (m, 2H), 7.40-7.31 (m, 2H), 6.86 (d, J=7.8 Hz, 1H), 3.83-3.71 (m, 3H), 2.66-2.54 (m, 2H), 1.80 (d, J=12.6 Hz, 1H), 1.35 (d, J=3.0 Hz, 2H), 1.25 (t, J=12.1 Hz, 1H), 1.05-0.93 (m, 2H), 0.86 (s, 1H), 0.47 (td, J=8.6, 4.7 Hz, 1H), 0.43-0.34 (m, 2H), 0.21-0.07 (m, 2H).

### Example 14. Synthesis of N-(1-(1H-tetrazol-5-yl)azocan-3-yl)-1-(4-chlorophenyl)cyclopropane-1-carboxamide (Compound 14, rac-14) and Compounds 14A* and 14B*

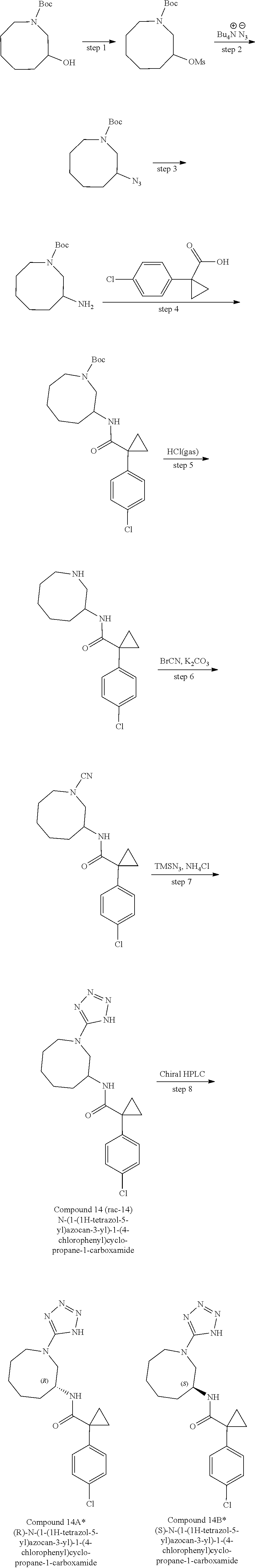

### Example 14Follows Protocol C

Step 1: To a stirred solution of tert-butyl 3-hydroxyazocane-1-carboxylate (350 mg, 1.53 mmol, 1 equiv) and pyridine (363.07 mg, 4.59 mmol, 3 equiv) in dichloromethane (DCM) (15 mL) was added methanesulfonic anhydride (398.79 mg, 2.29 mmol, 1.5 equiv) at 0° C. under nitrogen atmosphere. The final reaction mixture was stirred for 2 h at room temperature. The resulting mixture was diluted with water (50 mL). The resulting mixture was extracted with ethyl acetate (EtOAc) (3×50 mL). The combined organic layers were washed with brine (1×50 mL), dried over anhydrous Na₂SO₄. After filtration, the filtrate was concentrated under reduced pressure. The residue was purified by Prep-TLC (petroleum ether:ethyl acetate 1:1) to provide tert-butyl 3-(methanesulfonyloxy)azocane-1-carboxylate (230 mg, 49% yield). LCMS (ES, m/z): RT=1.06 min, m/z=308[M+H]⁺.

Step 2: A mixture of tert-butyl 3-(methanesulfonyloxy)azocane-1-carboxylate (230 mg, 0.75 mmol, 1 equiv) and tetrabutylammonium azide (319.28 mg, 1.12 mmol, 1.5 equiv) in dimethyl formamide (DMF) (7.0 mL) was stirred overnight at 100° C. The mixture was allowed to cool down to room temperature. The resulting mixture was diluted with water (70 mL). The resulting mixture was extracted with ethyl acetate (EtOAc) (3×100 mL). The combined organic layers were washed with brine (1×100 mL), and dried over anhydrous Na₂SO₄. After filtration, the filtrate was concentrated under reduced pressure. This resulted in tert-butyl 3-azidoazocane-1-carboxylate (140 mg, 74% yield). LCMS (ES, m/z): RT=1.29 min, m/z=255[M+H]⁺.

Step 3: A solution of tert-butyl 3-azidoazocane-1-carboxylate (150 mg, 0.59 mmol, 1 equiv) and Pd/C (50 mg, 0.47 mmol, 0.8 equiv) in methanol (MeOH) (5.0 mL) was stirred for 2 h at room temperature. The resulting mixture was filtered, the filter cake was washed with methanol (MeOH) (3×20 mL). The filtrate was concentrated under reduced pressure. This resulted in tert-butyl 3-aminoazocane-1-carboxylate (100 mg, 74% yield). LCMS (ES, m/z): RT=0.62 min, m/z=229[M+H]⁺.

Step 4: To a stirred solution of tert-butyl 3-aminoazocane-1-carboxylate (“amine (iv) reagent”) (110 mg, 0.48 mmol, 1.1 equiv) and 1-(4-chlorophenyl)cyclopropane-1-carboxylic acid (“carboxyl (ii) reagent”) (86.11 mg, 0.44 mmol, 1 equiv) in dimethyl formamide (DMF) (5 mL) was added triethylamine (TEA) (132.95 mg, 1.31 mmol, 3 equiv) followed by (1-[bis(dimethylamino)methylene]-1H-1,2,3-triazolo[4,5-b]pyridinium 3-oxide hexafluorophosphate (HATU) (183.17 mg, 0.48 mmol, 1.1 equiv). The resulting mixture was stirred for 1 h at room temperature under nitrogen atmosphere. The resulting mixture was diluted with water (50 mL). The resulting mixture was extracted with ethyl acetate (EtOAc) (3×50 mL). The combined organic layers were washed with brine (1×50 mL), dried over anhydrous Na₂SO₄. After filtration, the filtrate was concentrated under reduced pressure. The residue was purified by silica gel column chromatography, eluting with dichloromethane (DCM)/petroleum ether (PE) (1:5), to provide tert-butyl 3-[1-(4-chlorophenyl)cyclopropaneamido]azocane-1-carboxylate (110 mg, 40% yield). LCMS (ES, m/z): RT=1.22 min, m/z=407[M+H]⁺.

Step 5: A solution of tert-butyl 3-[1-(4-chlorophenyl)cyclopropaneamido]azocane-1-carboxylate (110 mg, 0.27 mmol, 1 equiv) in dichloromethane (DCM) (3.0 mL) was bubbled with HCl (gas). The resulting mixture was stirred for 1 h at room temperature. The resulting mixture was concentrated under reduced pressure. This resulted in N-(azocan-3-yl)-1-(4-chlorophenyl)cyclopropane-1-carboxamide (80 mg, 66% yield). LCMS (ES, m/z): RT=0.69 min, m/z=307[M+H]⁺.

Step 6: To a stirred solution of N-(azocan-3-yl)-1-(4-chlorophenyl)cyclopropane-1-carboxamide (80 mg, 0.26 mmol, 1 equiv) in acetonitrile (MeCN) (3.0 mL) was added K₂CO₃ (108.10 mg, 0.78 mmol, 3 equiv) and cyanogen bromide (138.09 mg, 1.31 mmol, 5 equiv). The resulting mixture was stirred for 2 h at 80° C. The resulting mixture was diluted with water (20 mL), and then extracted with EtOAc (3×20 mL). The combined organic layers were washed with brine (1×20 mL), dried over anhydrous Na₂SO₄, filtered, and the filtrate was concentrated under reduced pressure. The residue was purified by reverse flash chromatography (C18 silica gel; mobile phase, Acetonitrile (MeCN) in water (10 mmol/L NH₄HCO₃), 10% to 50% gradient in 10 min; detector, UV 254 nm) to provide 1-(4-chlorophenyl)-N-(1-cyanoazocan-3-yl)cyclopropane-1-carboxamide (51 mg, 59% yield). LCMS (ES, m/z): RT=0.93 min, m/z=332 [M+H]⁺.

Step 7: To a stirred solution of 1-(4-chlorophenyl)-N-(1-cyanoazocan-3-yl)cyclopropane-1-carboxamide (90 mg, 0.27 mmol, 1 equiv) in dimethyl formamide (DMF) (2 mL) was added trimethylsilyl azide (62.49 mg, 0.54 mmol, 2 equiv) and NH₄Cl (43.52 mg, 0.81 mmol, 3 equiv). The resulting mixture was stirred overnight at 120° C. The resulting mixture was concentrated under reduced pressure. The crude product was purified by PREP HPLC (XBridge Shield RP18 OBD Column, 30*150 mm, 5 μm; mobile phase, water (10 mmol/L NH₄HCO₃) and acetonitrile (MeCN) (21% acetonitrile (MeCN) up to 38% in 8 min); Detector, UV 254 nm, to provide N-(1-(1H-tetrazol-5-yl)azocan-3-yl)-1-(4-chlorophenyl)cyclopropane-1-carboxamide (Compound 14, rac-14) as a mixture of two stereoisomers.

Step 8. The crude product, rac-14, was purified by Chiral-PREP HPLC (CHIRAL ART Cellulose-SB, 2*25 cm, 5 μm; mobile phase, Hex- and EtOH- (hold 10% EtOH- in 15 min); Detector, UV 254 nm, to provide Compound 14A* (8.7 mg) as first eluting peak and Compound 14B* (6.8 mg) as second eluting peak, stereochemistry arbitrarily assigned.

Compound 14A*: LCMS (ES, m/z): RT=1.47 min, m/z=375[M+H]⁺; ¹H NMR (400 MHz, DMSO-d6) δ 7.43-7.29 (m, 4H), 7.18 (d, J=8.1 Hz, 1H), 3.97 (s, 1H), 3.44 (q, J=4.0, 3.1 Hz, 3H), 1.75-1.61 (m, 2H), 1.54 (d, J=9.2 Hz, 2H), 1.44 (t, J=10.2 Hz, 2H), 1.37 (d, J=2.5 Hz, 2H), 1.30-1.22 (m, 2H), 1.05-0.95 (m, 2H).

Compound 14B*: LCMS (ES, m/z): RT=1.47 min, m/z=375[M+H]⁺; ¹H NMR (400 MHz, DMSO-d6) δ 7.41-7.31 (m, 4H), 7.17 (d, J=7.9 Hz, 1H), 3.97 (d, J=8.5 Hz, 1H), 3.48-3.42 (m, 3H), 1.77-1.61 (m, 2H), 1.53 (t, J=9.0 Hz, 2H), 1.48-1.39 (m, 2H), 1.37 (d, J=2.6 Hz, 2H), 1.31-1.22 (m, 2H), 1.04-0.93 (m, 2H).

### Example 15. Synthesis of (R)—N-(1-(1H-tetrazol-5-yl)piperidin-3-yl)-1-(4-chloro-2-methoxyphenyl)cyclopropane-1-carboxamide (Compound 15A-OMe), (R)—N-(1-(1H-tetrazol-5-yl)piperidin-3-yl)-1-(4-chloro-2-hydroxyphenyl)cyclopropane-1-carboxamide (Compound 15A), and Compounds 15B—OMe and 15B—OMe

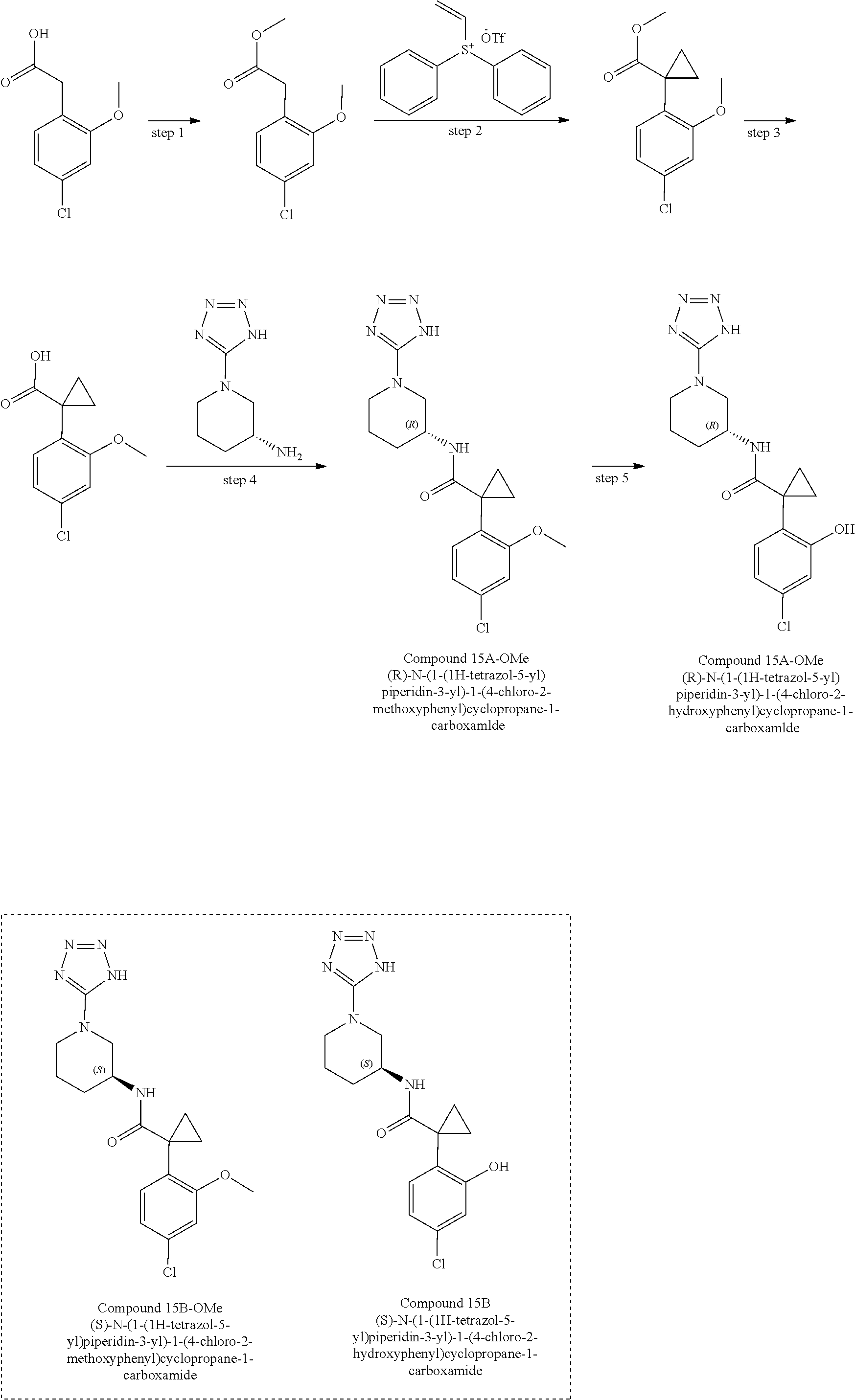

### Example 15 Follows Protocol A

Step 1: Into a 40 mL vial was added (4-chloro-2-methoxyphenyl)acetic acid (1 g, 4.99 mmol, 1 equiv), methanol (MeOH) (10 mL), and H₂SO₄ (2 mL) at room temperature. The resulting mixture was stirred for 2 h at 60° C. under nitrogen atmosphere. The aqueous layer was extracted with ethyl acetate (EtOAc) (3×100 mL). The resulting mixture was concentrated under reduced pressure to provide methyl 2-(4-chloro-2-methoxyphenyl)acetate (900 mg, 84% yield).

Step 2: Into an 8 mL vial was added methyl 2-(4-chloro-2-methoxyphenyl)acetate (200 mg, 0.932 mmol, 1 equiv), 1,8-diazabicyclo[5.4.0]undec-7-one (DBU) (284 mg, 1.86 mmol, 2 equiv), dimethylsulfoxide (DMSO) (10 mL), and diphenyl vinylsulfonium triflate (371 mg, 1.03 mmol, 1.1 equiv) at room temperature. The resulting mixture was stirred for 2 h at room temperature under nitrogen atmosphere. The residue was purified by reverse phase flash chromatography (water: acetonitrile (MeCN)=1:1) to provide methyl 1-(4-chloro-2-methoxyphenyl)cyclopropane-1-carboxylate (190 mg, 85% yield).

Step 3: Into an 8 mL vial was added methyl 1-(4-chloro-2-methoxyphenyl)cyclopropane-1-carboxylate (200 mg, 0.831 mmol, 1 equiv), NaOH (66.5 mg, 1.66 mmol, 2 equiv), methanol (MeOH) (5 mL), and water (5 mL) at room temperature. The resulting mixture was stirred for 5 h at room temperature under nitrogen atmosphere. The mixture was acidified to pH 3 with conc. HCl. The aqueous layer was extracted with ethyl acetate (EtOAc) (3×50 mL), dried, and concentrated under reduced pressure to provide 1-(4-chloro-2-methoxyphenyl)cyclopropane-1-carboxylic acid (150 mg, 80% yield).

Step 4: Into a 20 mL vial was added 1-(4-chloro-2-methoxyphenyl)cyclopropane-1-carboxylic acid (“carboxyl (ii) reagent”) (70.0 mg, 0.30 mmol, 1 equiv) and (3R)-1-(1H-1,2,3,4-tetrazol-5-yl)piperidin-3-amine (“amine (i) reagent”) (52 mg, 0.3 mmol, 1 equiv), N,N,N,N′-tetramethylchloroformamidinium hexafluorophosphate (TCFH) (95.3 mg, 0.34 mmol, 1.1 equiv), N-methyl imidazole (NMI) (76.1 mg, 0.92 mmol, 3 equiv), and acetonitrile (5 mL) at room temperature. The resulting mixture was stirred for 1 h at room temperature under nitrogen atmosphere. The reaction progress was monitored by LCMS. The residue was purified by reversed-phase flash chromatography (C18 silica gel; mobile phase, acetonitrile (MeCN) in water, 35% to 45% gradient in 10 min; detector, UV 220/254 nm) to provide (R)—N-(1-(1H-tetrazol-5-yl)piperidin-3-yl)-1-(4-chloro-2-methoxyphenyl)cyclopropane-1-carboxamide (Compound 15A-OMe) (60 mg, 52% yield). LCMS (ESI): RT=0.785 min, m/z=377[M+H]⁺.

Step 5: Into an 8 mL vial was added Compound 15A-OMe (60 mg, 0.15 mmol, 1 equiv), dichloromethane (DCM) (3 mL), and BBr₃ (199.43 mg, 0.75 mmol, 5 equiv) at 0° C. The resulting mixture was stirred for 2 h at room temperature under nitrogen atmosphere. The reaction progress was monitored by LCMS. The reaction was quenched by the addition of water (1 mL) at 0° C. The resulting mixture was concentrated under reduced pressure. The crude product was purified by PREP HPLC (XBridge Prep OBD C18 Column, 30* 150 mm, 5 μm; Mobile Phase A: water(10 mmol/L NH₄HCO₃), Mobile Phase B: acetonitrile (MeCN); Flow rate: 60 mL/min; Gradient: 17% B to 27% B in 8 min, 27% B; Wave Length: 254 nm; RT(min): 7) to provide (R)—N-(1-(1H-tetrazol-5-yl)piperidin-3-yl)-1-(4-chloro-2-hydroxyphenyl)cyclopropane-1-carboxamide (Compound 15A) (9.2 mg). LCMS (ESI): RT=0.640 min, m/z=363.1[M+H]⁺. ¹H NMR (400 MHz, Methanol-d4) δ 7.18 (d, J=8.6 Hz, 1H), 6.81-6.80 (m, 2H), 4.01 (s, 1H), 3.40 (d, J=12.2 Hz, 1H), 3.25 (s, 2H), 3.18-3.09 (m, 1H), 1.71 (s, 2H), 1.58 (td, J=14.9, 14.4, 9.6 Hz, 2H), 1.50 (d, J=3.5 Hz, 2H), 1.00 (q, J=3.1 Hz, 2H).

Compound 15B—OMe and Compound 15 may be synthesized following Example 15 using (3S)-1-(1H-1,2,3,4-tetrazol-5-yl)piperidin-3-amine instead of (3R)-1-(1H-1,2,3,4-tetrazol-5-yl)piperidin-3-amine in step 4.

### Example 16. Synthesis of 1-(2-methoxy-4-(trifluoromethyl)phenyl)-N-((3R,5S)-5-methyl-1-(1H-tetrazol-5-yl)piperidin-3-yl)cyclopropane-1-carboxamide (Compound 16A-OMe) and 1-(2-hydroxy-4-(trifluoromethyl)phenyl)-N-((3R,5S)-5-methyl-1-(1H-tetrazol-5-yl)piperidin-3-yl)cyclopropane-1-carboxamide (Compound 16A), and Compounds 16B, 16C, 16D, 16B—OMe, 16C—OMe, and 16D-OMe

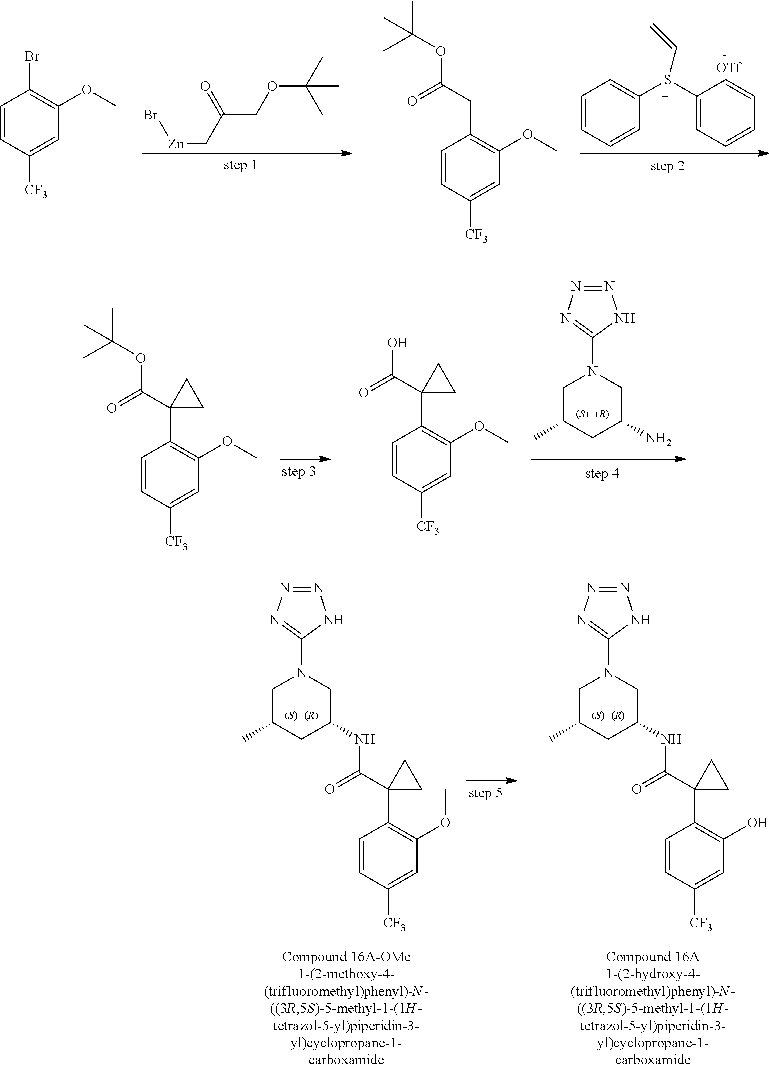

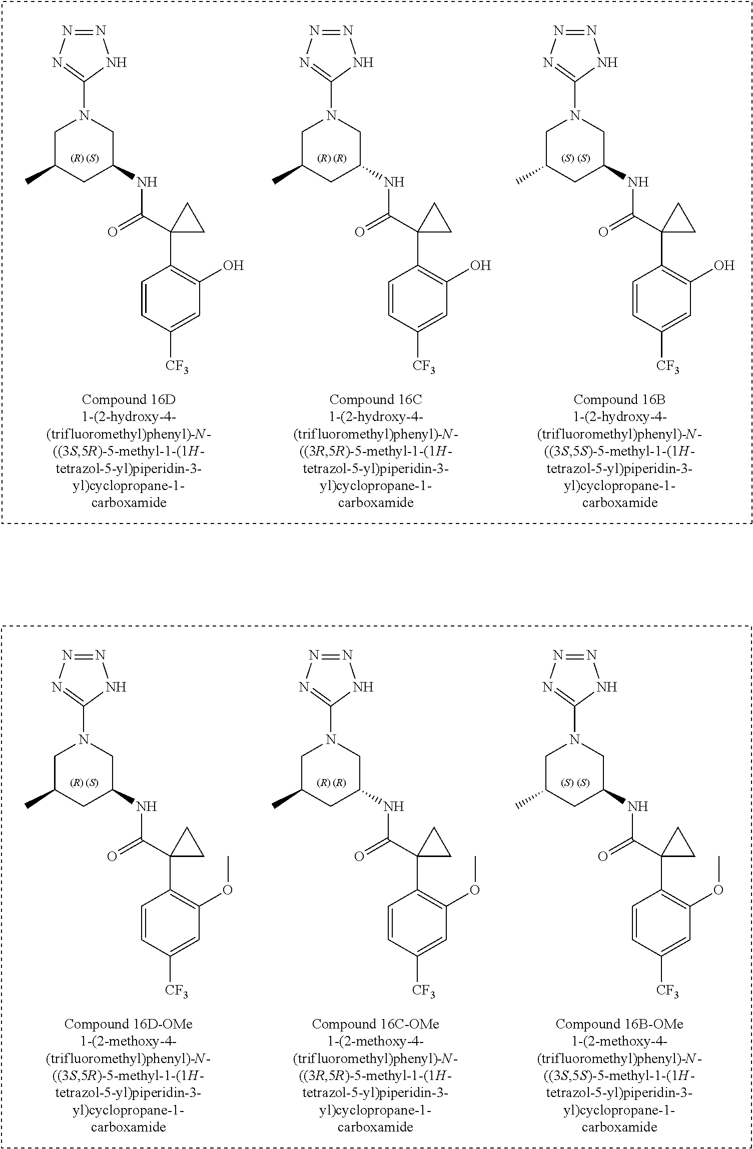

### Example 16 Follows Protocol A

Step 1: A solution of 1-bromo-2-methoxy-4-(trifluoromethyl)benzene (1000 mg, 3.921 mmol, 1 equiv), 1-(bromozincio)-3-(tert-butoxy)propan-2-one (3228.51 mg, 11.763 mmol, 3 equiv), and bis(tri-tert-butylphosphine)palladium(0) (Pd[(t-Bu)₃P]₂) (601.16 mg, 1.176 mmol, 0.3 equiv) in tetrahydrofuran (THF) was stirred for 2 h at 60° C. under nitrogen atmosphere. The resulting mixture was extracted with ethyl acetate (EtOAc) (3×10 mL). The combined organic layers were washed with water (3×10 mL), dried over anhydrous Na₂SO₄. After filtration, the filtrate was concentrated under reduced pressure. The residue was purified by silica gel column chromatography, eluting with petroleum ether:ethyl acetate (8:1) to provide tert-butyl 2-[2-methoxy-4-(trifluoromethyl)phenyl]acetate (800 mg, 70% yield). LCMS (ES, m/z): RT=0.576 min, m/z=290.0[M+H]⁺.

Step 2: A solution of tert-butyl 2-[2-methoxy-4-(trifluoromethyl)phenyl]acetate (800 mg, 2.756 mmol, 1 equiv), ethenyldiphenylsulfanium (764.27 mg, 3.583 mmol, 1.3 equiv) and 1,8-diazabicyclo[5.4.0]undec-7-ene (DBU) (1258.70 mg, 8.268 mmol, 3 equiv) in dimethylsulfoxide (DMSO) was stirred for 1 h at 25° C. under nitrogen atmosphere. The residue was purified by reverse flash chromatography (C18 silica gel; mobile phase, acetonitrile (MeCN) in water, 60% to 70% gradient in 10 min; detector, UV 254 nm) to provide tert-butyl 1-[2-methoxy-4-(trifluoromethyl)phenyl]cyclopropane-1-carboxylate (600 mg, 69% yield). LCMS (ES, m/z): RT=1.025 min, m/z=316[M+H]⁺.

Step 3: A solution of tert-butyl 1-(2-methoxy-4-methylphenyl)cyclopropane-1-carboxylate (600 mg, 2.287 mmol, 1 equiv) and trifluoroacetic acid (TFA) (5215.48 mg, 45.740 mmol, 20 equiv) in dichloromethane (DCM) was stirred for 2 h at room temperature under air atmosphere. The resulting mixture was concentrated under reduced pressure. The residue was purified by reverse flash chromatography (C18 silica gel; mobile phase, Acetonitrile (MeCN) in water (0.1% FA), 40% to 50% gradient in 10 min; detector, UV 254 nm) to provide 1-[2-methoxy-4-(trifluoromethyl)phenyl]cyclopropane-1-carboxylic acid (500 mg, 84% yield). LCMS (ES, m/z): RT=0.607 min, m/z=260[M+H]⁺.

Step 4: A solution of 1-[2-methoxy-4-(trifluoromethyl)phenyl]cyclopropane-1-carboxylic acid (“carboxyl (ii) reagent”) (200 mg, 0.769 mmol, 1 equiv), (3R,5S)-5-methyl-1-(1H-1,2,3,4-tetrazol-5-yl)piperidin-3-amine (“amine (i) reagent”) (280.13 mg, 1.538 mmol, 2 equiv), N,N,N′,N′-tetramethylchloroformamidinium hexafluorophosphate (TCFH) (150.96 mg, 0.538 mmol, 0.7 equiv), and N-methyl imidazole (NMI) (252.43 mg, 3.076 mmol, 4 equiv) in acetonitrile (MeCN) was stirred for 2 h at room temperature under nitrogen atmosphere. The residue was purified by reverse flash chromatography (C18 silica gel; mobile phase, Acetonitrile (MeCN) in water, 20% to 30% gradient in 10 min; detector, UV 254 nm) to provide 1-(2-methoxy-4-(trifluoromethyl)phenyl)-N-((3R,5S)-5-methyl-1-(1H-tetrazol-5-yl)piperidin-3-yl)cyclopropane-1-carboxamide (Compound 16A-OMe). LCMS (ES, m/z): RT=0.519 min, m/z=425[M+H]⁺.

Step 5: A solution of Compound 16-OMe (50 mg, 0.118 mmol, 1 equiv) and (ethylsulfanyl)sodium (297.26 mg, 3.540 mmol, 30 equiv) in dimethylsulfoxide (DMSO) was stirred for 2 h at 120° C. under nitrogen atmosphere. The resulting mixture was extracted with ethyl acetate (EtOAc) (3×100 mL). The combined organic layers were washed with water (3×10 mL), dried over anhydrous Na₂SO₄. After filtration, the filtrate was concentrated under reduced pressure. The residue was purified by reverse flash chromatography (C18 silica gel; mobile phase, acetonitrile (MeCN) in water, 20% to 30% gradient in 10 min; detector, UV 254 nm) to provide a crude product (50 mg), which was further purified by PREP HPLC (30% Acetonitrile in water, isocratic) to provide 1-(2-hydroxy-4-(trifluoromethyl)phenyl)-N-((3R,5S)-5-methyl-1-(1H-tetrazol-5-yl)piperidin-3-yl)cyclopropane-1-carboxamide (Compound 16A) (9.8 mg, 20% yield). LCMS (ES, m/z): RT=1.381 min, m/z=410[M+H]⁺.

Compound 16B—OMe and Compound 16B may be synthesized from (3S,5S)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 4 of Example 16.

Compound 16C—OMe and Compound 16C may be synthesized from (3R,5R)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 4 of Example 16.

Compound 16D-OMe and Compound 16D may be synthesized from (3S,5R)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 4 of Example 16.

### Example 17. Synthesis of 1-(2-methoxy-4-(trifluoromethoxy)phenyl)-N-((3R,5S)-5-methyl-1-(1H-tetrazol-5-yl)piperidin-3-yl)cyclopropane-1-carboxamide (Compound 17A-OMe), 1-(2-hydroxy-4-(trifluoromethoxy)phenyl)-N-((3R,5S)-5-methyl-1-(1H-tetrazol-5-yl)piperidin-3-yl)cyclopropane-1-carboxamide (Compound 17A), and Compounds 17B, 17C, 17D, 17B—OMe, 17C—OMe, and 17D-OMe

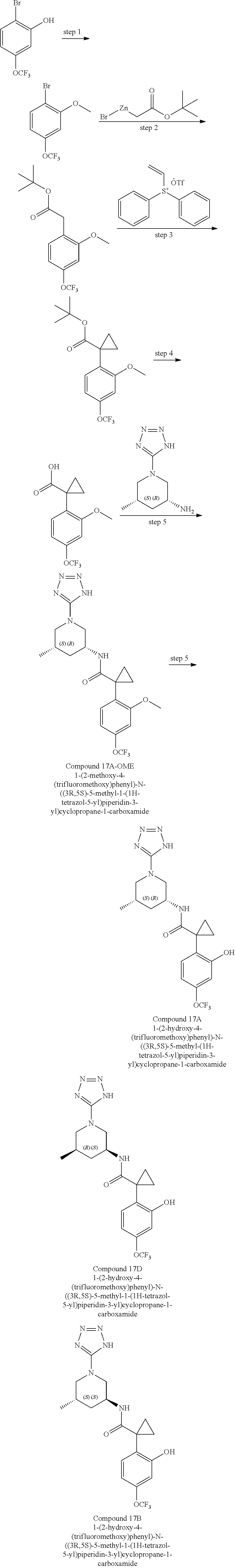

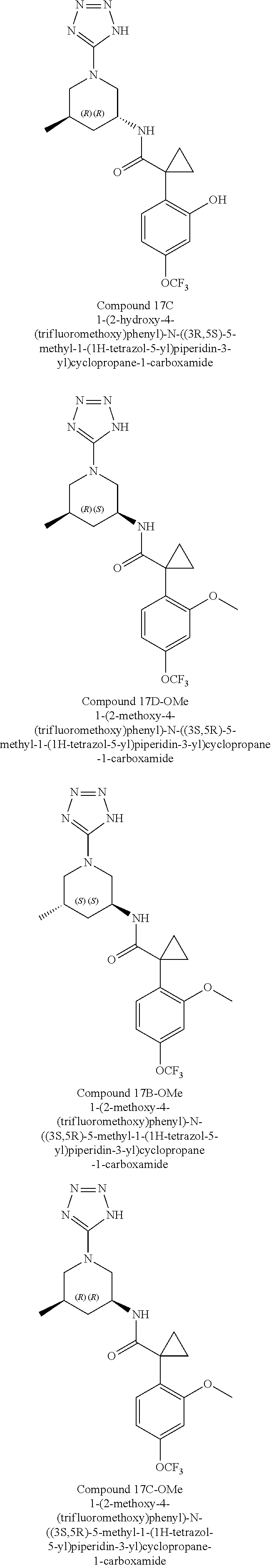

### Example 17 Follows Protocol A

Step 1: Into a 20 mL vial was added 2-bromo-5-(trifluoromethoxy)phenol (500 mg, 1.945 mmol, 1 equiv), K₂CO₃ (806.63 mg, 5.835 mmol, 3 equiv), methyl iodide (MeI) (552.28 mg, 3.890 mmol, 2 equiv), acetonitrile (MeCN) (5 mL) at room temperature. The resulting mixture was stirred for 1 h at 80° C. under nitrogen atmosphere. The aqueous layer was extracted with ethyl acetate (EtOAc) (3×50 mL).The residue was purified by silica gel column chromatography, eluting with petroleum ether:ethyl acetate (8:1) to provide 1-bromo-2-methoxy-4-(trifluoromethoxy)benzene (400 mg, 76% yield).

Step 2: Into a 20 mL vial was added 1-bromo-2-methoxy-4-(trifluoromethoxy)benzene (500 mg, 1.845 mmol, 1 equiv), tert-butyl 2-(bromozincio)acetate (960.92 mg, 3.690 mmol, 2 equiv), tetrahydrofuran (THF) (5 mL), 1,2,3,4,5-pentaphenyl-1′-(di-tert-butylphosphino)ferrocene (655.55 mg, 0.922 mmol, 0.5 equiv), and tert-butyl 2-[2-methoxy-4-(trifluoromethoxy)phenyl]acetate (400 mg) at room temperature. The resulting mixture was stirred overnight at 70° C. under nitrogen atmosphere. The aqueous layer was extracted with ethyl acetate (EtOAc) (3×50 mL). The residue was purified by silica gel column chromatography, eluting with petroleum ether:ethyl acetate (12:1), to provide tert-butyl 2-[2-methoxy-4-(trifluoromethoxy)phenyl]acetate (400 mg, 71% yield).

Step 3: Into an 8 mL vial was added tert-butyl 2-[2-methoxy-4-(trifluoromethoxy)phenyl]acetate (200 mg, 0.653 mmol, 1 equiv), 1,8-diazabicyclo[5.4.0]undec-7-ene (DBU) (298.24 mg, 1.959 mmol, 3 equiv), dimethylsulfoxide (DMSO) (5 mL), and diphenylvinylsulfonium triflate (354.95 mg, 0.980 mmol, 1.5 equiv) at room temperature. The resulting mixture was stirred for 2 h at room temperature under nitrogen atmosphere. The residue was purified by reverse flash chromatography (C18 silica gel; mobile phase, acetonitrile (MeCN) in water (0.1% NH₃ in water), 50% to 80% gradient in 10 min; detector, UV 254 nm) to provide tert-butyl 1-[2-methoxy-4-(trifluoromethoxy)phenyl]cyclopropane-1-carboxylate (150 mg, 69% yield).

Step 4: Into an 8 mL vial was added tert-butyl 1-[2-methoxy-4-(trifluoromethoxy)phenyl]cyclopropane-1-carboxylate (200 mg, 0.602 mmol, 1 equiv), dichloromethane (DCM) (2 mL), and trifluoroacetic acid (TFA) (0.5 mL) at room temperature. The resulting mixture was stirred for 2 h at room temperature under nitrogen atmosphere, then concentrated under vacuum to provide 1-[2-methoxy-4-(trifluoromethoxy)phenyl]cyclopropane-1-carboxylic acid (150 mg, 90% yield).

Step 5: Into an 8 mL vial was added 1-[2-hydroxy-4-(trifluoromethoxy)phenyl]cyclopropane-1-carboxylic acid (“carboxyl (ii) reagent”) (100 mg, 0.381 mmol, 1 equiv), (3R,5S)-5-methyl-1-(1H-1,2,3,4-tetrazol-5-yl)piperidin-3-amine (“amine (i) reagent”) (72.57 mg, 0.398 mmol, 1.1 equiv), triethylamine (TEA) (109.91 mg, 1.086 mmol, 3 equiv), and dimethyl formamide (DMF) (2 mL) at room temperature. The resulting mixture was stirred for 1 h at room temperature under nitrogen atmosphere. The residue was purified by reverse flash chromatography (C18 silica gel; mobile phase, acetonitrile (MeCN) in water (0.1% NH₃ in water), 10% to 50% gradient in 10 min; detector, UV 254 nm) to provide 1-(2-methoxy-4-(trifluoromethoxy)phenyl)-N-((3R,5S)-5-methyl-1-(1H-tetrazol-5-yl)piperidin-3-yl)cyclopropane-1-carboxamide (Compound 17A-OMe).

Step 6: Into an 8 mL vial was added Compound 17A-OMe (50 mg, 0.114 mmol, 1 equiv), dimethylsulfoxide (DMSO) (2 mL) and (ethylsulfanyl)sodium (95.49 mg, 1.140 mmol, 10 equiv) at room temperature. The resulting mixture was stirred for 1 h at 100° C. under nitrogen atmosphere. The residue was purified by reverse flash chromatography (C18 silica gel; mobile phase, acteonitrile (MeCN) in water (0.1% NH₃ in water), 10% to 50% gradient in 10 min; detector, UV 254 nm) to provide 1-(2-hydroxy-4-(trifluoromethoxy)phenyl)-N-((3R,5S)-5-methyl-1-(1H-tetrazol-5-yl)piperidin-3-yl)cyclopropane-1-carboxamide (Compound 17A) (19.1 mg). LCMS (ESI): RT=1.537 min, m/z=427[M+H]⁺. ¹H NMR (400 MHz, Methanol-d4) δ 7.32 (d, J=9.0 Hz, 1H), 6.79-6.73 (m, 2H), 3.99-3.83 (m, 2H), 3.79 (dd, J=12.6, 4.2 Hz, 1H), 2.73 (dd, J=12.0, 10.7 Hz, 1H), 2.56 (dd, J=12.8, 11.3 Hz, 1H), 1.90 (d, J=12.9 Hz, 1H), 1.53 (q, J=3.3 Hz, 2H), 1.18-1.07 (m, 1H), 1.05 (tt, J=4.7, 2.6 Hz, 2H), 0.97 (d, J=6.6 Hz, 3H).

Compound 17B—OMe and Compound 17B may be synthesized from (3S,5S)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 5 of Example 17.

Compound 17C—OMe and Compound 17C may be synthesized from (3R,5R)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 4 of Example 17.

Compound 17D-OMe and Compound 17D may be synthesized from (3S,5R)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 4 of Example 17.

### Example 18. Synthesis of 1-(4-chloro-3-fluoro-2-methoxyphenyl)-N-(5-methyl-1-(1H-tetrazol-5-yl)azepan-3-yl)cyclopropane-1-carboxamide (Compound 18-OMe, rac-18-OMe), 1-(4-chloro-3-fluoro-2-hydroxyphenyl)-N-(5-methyl-1-(1H-tetrazol-5-yl)azepan-3-yl)cyclopropane-1-carboxamide (Compound 18, rac-18), Compounds 18A-OMe*, 18B—OMe*, 18C—OMe*, and 18D-OMe*, and Compounds 18A*, 18B*, 18C*, and 18D*

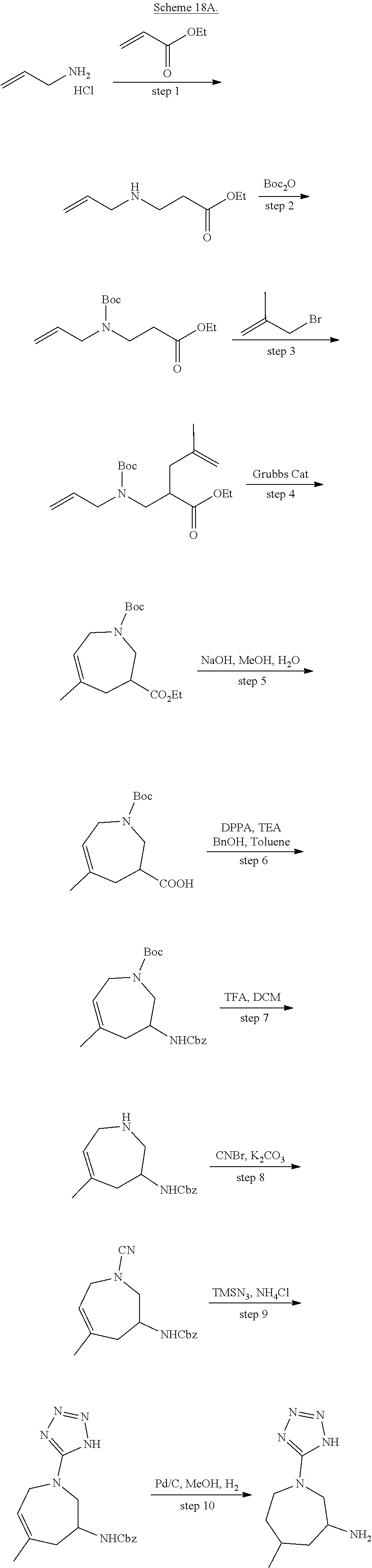

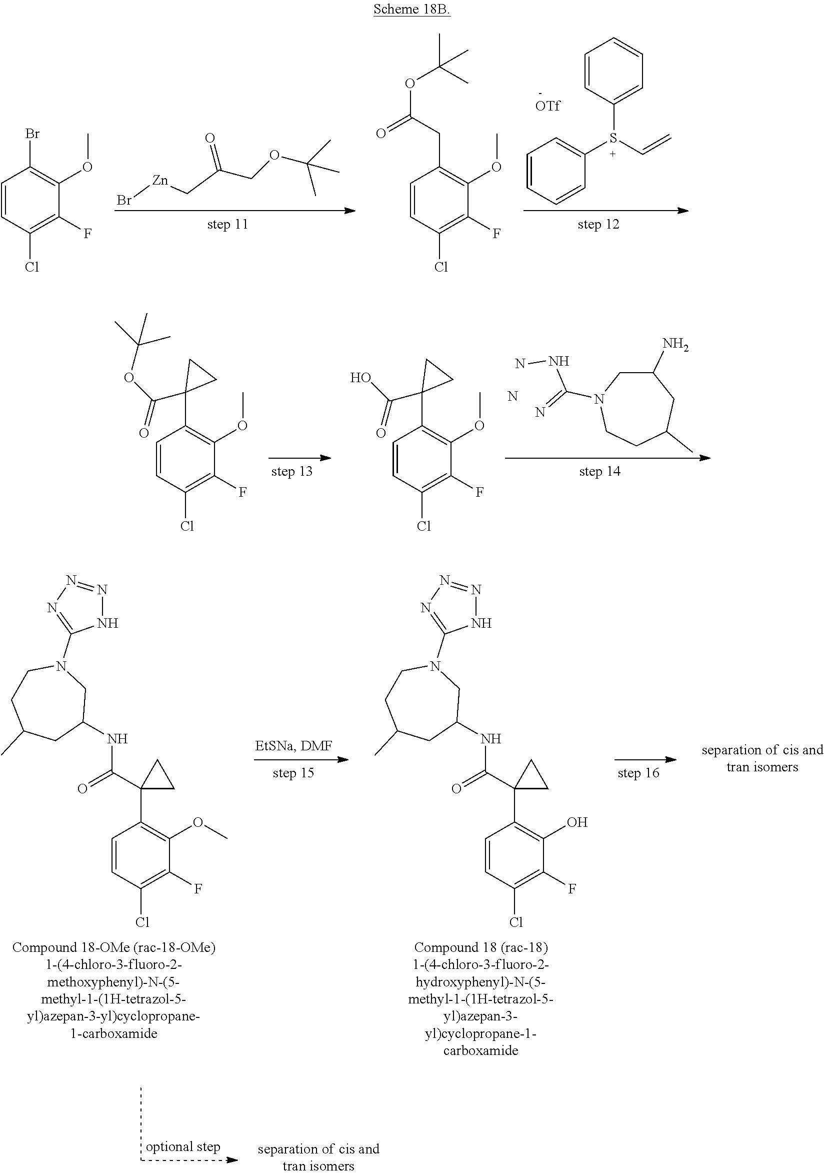

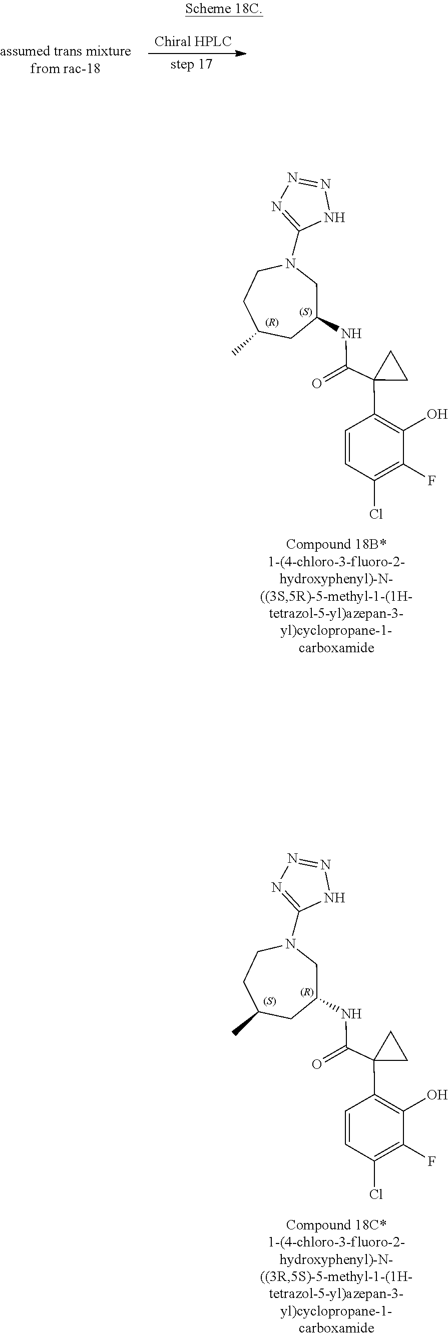

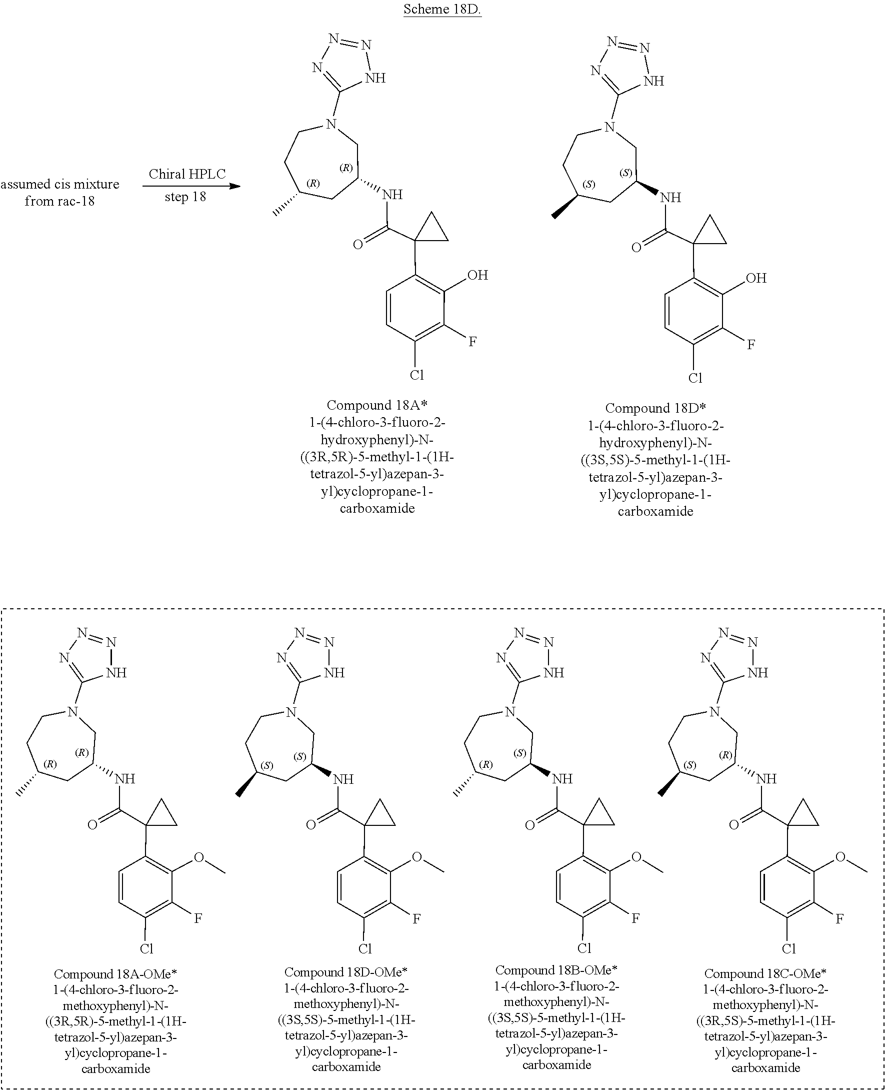

### Example 18 Follows Protocol A

Step 1: Into a 1000 mL round-bottom flask was added allylamine hydrochloride (25.0 g, 267.23 mmol, 1 equiv), ethanol (EtOH) (400 mL), ethyl acrylate (32.10 g, 320.68 mmol, 1.2 equiv) and 1,8-diazabicyclo[5.4.0]undec-7-ene (DBU) (81.36 g, 534.47 mmol, 2 equiv) at room temperature. The resulting mixture was stirred for 3 h at room temperature under nitrogen atmosphere. The resulting mixture was concentrated under reduced pressure. The reaction was quenched by the addition of water (100 mL) at room temperature, and the aqueous layer was extracted with ethyl acetate (EtOAc) (3×500 mL), the organic layer was dried over anhydrous Na₂SO₄, and the resulting mixture was concentrated under reduced pressure to provide ethyl 3-(prop-2-en-1-ylamino)propanoate (26 g, 62% yield) which was used in the next step without further purification. LCMS (ES, m/z): RT=0.236 min, m/z=158[M+1]+.

Step 2: Into a 500 mL 3-necked round-bottom flask was added ethyl 3-(prop-2-en-1-ylamino)propanoate (25.0 g, 159.02 mmol, 1 equiv), di-tert-butyl dicarbonate (69.41 g, 318.04 mmol, 2 equiv), and dichloromethane (DCM) (300 mL) at 0° C. The resulting mixture was stirred for 2 h at room temperature, then the reaction was quenched by the addition of ice water (200 mL) at 0° C. The aqueous layer was extracted with ethyl acetate (EtOAc) (3×500 mL), and the organic layer was dried over anhydrous Na₂SO₄. The resulting mixture was concentrated under reduced pressure, and the residue was purified by silica gel column chromatography, eluting with petroleum ether:ethyl acetate (12:1) to provide ethyl 3-[(tert-butoxycarbonyl)(prop-2-en-1-yl)amino]propanoate (23 g, 56% yield). LCMS (ES, m/z): RT=0.993 min, m/z=258[M+1]+.

Step 3: To a stirred solution of ethyl 3-[(tert-butoxycarbonyl)(prop-2-en-1-yl)amino]propanoate (20 g, 77.72 mmol, 1 equiv) in tetrahydrofuran (THF) (300 mL) was added lithium bis(trimethylsilyl) amide (LiHMDS) (15.61 g, 93.26 mmol, 1.2 equiv) dropwise at −78° C. under nitrogen atmosphere. The resulting mixture was stirred for 30 min at −78° C. under nitrogen atmosphere. 3-Bromo-2-methylprop-1-ene (20.82 g, 155.44 mmol, 2 equiv) was then added dropwise at −78° C. under nitrogen atmosphere. The resulting mixture was stirred for 1 h at 0° C. under nitrogen atmosphere. The reaction was quenched by the addition of sat. NH₄Cl (aq.) (500 mL) at 0° C. The aqueous layer was extracted with ethyl acetate (EtOAc) (3×500 mL). The organic layer was dried over anhydrous Na₂SO₄. The resulting mixture was concentrated under reduced pressure. The residue was purified by silica gel column chromatography, eluting with petroleum ether:ethyl acetate (12:1) to provide ethyl 2-{[(tert-butoxycarbonyl)(prop-2-en-1-yl)amino]methyl}pent-4-enoate (15 g, 65% yield). LCMS (ES, m/z): RT=1.097 min, m/z=312[M+1]+.

Step 4: Into a 5000 mL 3-necked round-bottom flask was added ethyl 2-{1[(tert-butoxycarbonyl)(prop-2-en-1-yl)amino]methyl}-4-methylpent-4-enoate (40 g, 128.44 mmol, 1 equiv), tetrahydrofuran (4000 mL), (1,3-bis(2,4,6-trimethylphenyl)-2-imidazolidinylidene) dichloro(phenylmethylene) (tricyclohexylphosphine)ruthenium (Grubbs 2nd generation catalyst) (16.36 g, 19.26 mmol, 0.15 equiv) at room temperature. The resulting mixture was stirred for 2 h at 55° C. under nitrogen atmosphere. The resulting mixture was concentrated under reduced pressure. The reaction was quenched by the addition of water (500 mL). The aqueous layer was extracted with ethyl acetate (EtOAc) (3×300 mL). The residue was purified by silica gel column chromatography, eluting with petroleum ether:ethyl acetate (12:1), to provide 1-tert-butyl 3-ethyl 5-methyl-2,3,4,7-tetrahydroazepine-1,3-dicarboxylate) (30 g, 82% yield).

Step 5: Into a 500 mL round-bottom flask was added 1-tert-butyl 3-ethyl 5-methyl-2,3,4,7-tetrahydroazepine-1,3-dicarboxylate (20.0 g, 70.58 mmol, 1 equiv), methanol (MeOH) (100 mL), NaOH (14.11 g, 352.90 mmol, 5 equiv), and water (100 mL) at room temperature. The resulting mixture was stirred for 2 h at room temperature. The mixture was acidified to pH 6 with HCl (aq)(2M) at 0° C. The aqueous layer was extracted with ethyl acetate (EtOAc) (3×200 mL). The organic layer was dried over anhydrous Na₂SO₄. The resulting mixture was concentrated under reduced pressure to provide the crude product, 1-(tert-butoxycarbonyl)-5-methyl-2,3,4,7-tetrahydro-1H-azepine-3-carboxylic acid, which was used in the next step directly without further purification.

Step 6: Into a 500 mL 3-necked round-bottom flask was added 1-(tert-butoxycarbonyl)-5-methyl-2,3,4,7-tetrahydroazepine-3-carboxylic acid (20.0 g, 78.33 mmol, 1 equiv), toluene (250 mL), benzyl alcohol (25.41 g, 235.01 mmol, 3 equiv), diphenylphosphoryl azide (DPPA) (64.67 g, 235.01 mmol, 3 equiv) and triethylamine (TEA) (23.78 g, 235.01 mmol, 3 equiv) at room temperature. The resulting mixture was stirred for 3 h at 100° C. under nitrogen atmosphere. The resulting mixture was concentrated under reduced pressure. The aqueous layer was extracted with ethyl acetate (EtOAc) (3×500 mL). The residue was purified by reverse flash chromatography (C18 column; mobile phase, acetonitrile (MeCN) in water, 50% to 60% gradient in 10 min; detector, UV 254 nm) to provide tert-butyl 3-{[(benzyloxy)carbonyl]amino}-5-methyl-2,3,4,7-tetrahydroazepine-1-carboxylate (12 g, 43% yield). LCMS (ES, m/z): RT=1.065 min, m/z=361[M+1]+.

Step 7: Into a 100 mL round-bottom flask was added tert-butyl 3-{[(benzyloxy)carbonyl]amino}-5-methyl-2,3,4,7-tetrahydroazepine-1-carboxylate (10.0 g, 27.74 mmol, 1 equiv), dichloromethane (DCM) (25 mL) and trifluoroacetic acid (TFA) (5 mL) at room temperature. The resulting mixture was stirred for 1 h at room temperature. The resulting mixture was concentrated under reduced pressure. This resulted in benzyl (5-methyl-2,3,4,7-tetrahydro-1H-azepin-3-yl)carbamate (8 g), which was used in the next step directly without further purification. LCMS (ES, m/z): RT=0.563 min, m/z=261[M+1]⁺.

Step 8: Into a 250 mL round-bottom flask was added benzyl N-(5-methyl-2,3,4,7-tetrahydro-1H-azepin-3-yl)carbamate (8.0 g, 30.72 mmol, 1 equiv), acetonitrile (MeCN) (50 mL), K₂CO₃ (12.74 g, 92.18 mmol, 3 equiv) and BrCN (3.91 g, 36.87 mmol, 1.20 equiv) at room temperature. The resulting mixture was stirred for 1 h at room temperature under nitrogen atmosphere. The aqueous layer was extracted with ethyl acetate (EtOAc) (3×200 mL). The resulting mixture was concentrated under reduced pressure.

The residue was purified by silica gel column chromatography, eluting with petroleum ether:ethyl acetate (10:1) to provide benzyl N-(1-cyano-5-methyl-2,3,4,7-tetrahydroazepin-3-yl)carbamate (7 g, 80% yield). LCMS (ES, m/z): RT=0.837 min, m/z=286[M+1]+.

Step 9: Into a 50 mL round-bottom flask was added benzyl N-(1-cyano-5-methyl-2,3,4,7-tetrahydroazepin-3-yl)carbamate (5 g, 17.52 mmol, 1 equiv), NH₄Cl (2.81 g, 52.56 mmol, 3 equiv) and azidotrimethylsilane (6.06 g, 52.56 mmol, 3 equiv) at room temperature. The resulting mixture was stirred for 2 h at 100° C. under nitrogen atmosphere. The mixture was allowed to cool down to room temperature. The residue was purified by reverse flash chromatography (C18 column; mobile phase, Acetonitrile (MeCN) in water, 40% to 60% gradient in 10 min; detector, UV 254 nm) to provide benzyl (5-methyl-1-(1H-tetrazol-5-yl)-2,3,4,7-tetrahydro-1H-azepin-3-yl)carbamate (4 g, 70% yield). LCMS (ES, m/z): RT=0.773 min, m/z=329[M+1]+.

Step 10: Into a 250 mL round-bottom flask was added benzyl N-[5-methyl-1-(1H-1,2,3,4-tetrazol-5-yl)-2,3,4,7-tetrahydroazepin-3-yl]carbamate (4.0 g, 12.18 mmol, 1 equiv), methanol (MeOH) (100 mL) and Pd/C (1.30 g, 12.18 mmol) at room temperature. The resulting mixture was stirred overnight at 70° C. under hydrogen atmosphere. The resulting mixture was filtered, the filter cake was washed with methanol (MeOH) (3×20 mL). The filtrate was concentrated under reduced pressure to provide 5-methyl-1-(1H-tetrazol-5-yl)azepan-3-amine (2.5 g). LCMS (ES, m/z): RT=0.101 min, 0.289,m/z=197[M+1]+.

Step 11: Into a 40 mL sealed tube was added 1-bromo-4-chloro-3-fluoro-2-methoxybenzene (400 mg, 1.044 mmol, 1 equiv), tris(dibenzylideneacetone)dipalladium(0) (Pd₂(dba)₃) (265.20 mg, 0.209 mmol, 0.2 equiv) and tetrahydrofuran (THF) (15 mL) at room temperature. The resulting mixture was stirred for 10 h at 80° C. under nitrogen atmosphere. The resulting mixture was extracted with ethyl acetate (EtOAc) (3×250 mL), and dried over anhydrous Na₂SO₄. After filtration, the filtrate was concentrated under reduced pressure. The residue was purified by silica gel column chromatography, eluting with petroleum ether:ethyl acetate (10:1), to provide tert-butyl 2-(4-chloro-3-fluoro-2-methoxyphenyl)acetate (400 mg, 79% yield). GCMS (ESI): RT=8.5 min, m/z=274.

Step 12: Into a 40 mL sealed tube was added tert-butyl 2-(4-chloro-3-fluoro-2-methoxyphenyl)acetate (400 mg, 0.728 mmol, 1 equiv), ethenyldiphenylsulfanium (400.95 mg, 1.092 mmol, 1.5 equiv), 1,8-diazabicyclo[5.4.0]undec-7-ene (DBU) (660.06 mg, 2.184 mmol, 3 equiv) and dimethylsulfoxide (DMSO) (4 mL, 56.318 mmol, 77.36 equiv) at room temperature. The resulting mixture was stirred for 1 h at room temperature under air atmosphere. The resulting mixture was extracted with ethyl acetate (EtOAc) (3×250 mL), dried over anhydrous Na₂SO₄, filtered, and the filtrate was concentrated under reduced pressure. The crude product was purified by reverse phase flash chromatography (water: acetonitrile (MeCN)=2:8) to provide tert-butyl 1-(4-chloro-3-fluoro-2-methoxyphenyl)cyclopropane-1-carboxylate (200 mg, 55% yield). GCMS (ESI): RT=8.7 min, m/z=300.

Step 13: Into a 8 mL sealed tube was added tert-butyl 1-(4-chloro-3-fluoro-2-methoxyphenyl)cyclopropane-1-carboxylate (400 mg, 0.399 mmol, 1 equiv) and dichloromethane (DCM) (5 mL, 78.653 mmol, 197.12 equiv) at room temperature. The resulting mixture was stirred for 1 h at room temperature under air atmosphere, then concentrated to provide crude 1-(4-chloro-3-fluoro-2-methoxyphenyl)cyclopropane-1-carboxylic acid, which was used in the next step directly without further purification. LCMS (ESI): RT=0.84 min, m/z=245 [M+H]⁺.

Step 14: Into a 20 mL sealed tube was added 1-(4-chloro-3-fluoro-2-methoxyphenyl)cyclopropane-1-carboxylic acid (“carboxyl (ii) reagent”) (120 mg, 0.490 mmol, 1 equiv), 5-methyl-1-(1H-tetrazol-5-yl)azepan-3-amine (“amine (i) reagent”) (96 mg, 0.490 mmol, 1 equiv), N,N,N′,N′-tetramethylchloroformamidinium hexafluorophosphate (TCFH) (137.62 mg, 0.490 mmol, 1 equiv), N-methyl imidazole (NMI) (201.36 mg, 2.450 mmol, 5 equiv) and acetonitrile (MeCN) (6.67 mL, 126.699 mmol, 258.57 equiv) at room temperature. The resulting mixture was stirred for 1 h at room temperature under air atmosphere. The resulting mixture was concentrated under vacuum. The residue product was purified by reverse phase flash chromatography (water: acetonitrile (MeCN)=6:4) to provide 1-(4-chloro-3-fluoro-2-methoxyphenyl)-N-(5-methyl-1-(1H-tetrazol-5-yl)azepan-3-yl)cyclopropane-1-carboxamide (Compound 18-OMe, rac-18-OMe) (70 mg, 37% yield). LCMS (ESI): RT=0.74 min, m/z=423 [M+H]⁺.

Step 15: Into an 8 mL vial were added rac-18-OMe (70.0 mg, 0.170 mmol, 1 equiv), sodium ethanethiolate (EtSNa) (216 mg, 2.56 mmol, 15 equiv) and dimethyl formamide (DMF) (2 mL) at room temperature. The resulting mixture was stirred for 1 h at 120° C. The reaction was monitored by LCMS.

The reaction was quenched with water (2 mL) at room temperature. The resulting mixture was concentrated under vacuum. The residue was purified by reversed-phase flash chromatography (C18 silica gel; mobile phase, acetonitrile (MeCN) in water, 20% to 35% gradient in 10 min; detector, UV 220 nm) to provide 1-(4-chloro-3-fluoro-2-hydroxyphenyl)-N-(5-methyl-1-(1H-tetrazol-5-yl)azepan-3-yl)cyclopropane-1-carboxamide (Compound 18, rac-18) (55 mg, 73% purity).

Step 16: The crude rac-18 (55 mg) was purified by PREP HPLC (XBridge Prep Phenyl OBD Column, 19*150 mm, 5 μm; Mobile Phase A: Water (10 mmol/L NH₄HCO₃), Mobile Phase B: acetonitrile; Flow rate: 60 mL/min; Gradient: 25% B to 35% B in 8 min; Wave Length: 254 nm; RT(min): 8.5-10.5) to provide the assumed cis mixture (26 mg, 36% yield) as a first eluting peak, and the assumed trans mixture (16 mg, 23% yield) as a second eluting peak, stereochemistry arbitrarily assigned.

Step 17: the assumed trans mixture was separated using the following conditions (CHIRALPAK ID, 2*25 cm, 5 μm; Mobile Phase A: hexanes (0.1% trifluoroacetic acid (TFA)), Mobile Phase B: ethanol; Flow rate: 20 mL/min; Gradient: 12% B to 12% B in 16 min; Wave Length: 220/254 nm) to provide Compound 18B* (RT(min): 13.09, 10.6 mg) and Compound 18C* (RT(min): 15.32, 10.4 mg). Stereochemistry was arbitrarily assigned.

Compound 18B*: LCMS (ESI): RT=0.74 min, m/z=409 [M+H]⁺. ¹H NMR (400 MHz, DMSO-d6) δ 14.59 (s, 1H), 10.22 (s, 1H), 6.91 (d, J=8.6 Hz, 1H), 6.88-6.80 (m, 1H), 6.58 (d, J=8.0 Hz, 1H), 4.16 (q, J=6.4 Hz, 1H), 3.61-3.47 (m, 3H), 3.18 (ddd, J=13.8, 9.7, 4.4 Hz, 1H), 1.77 (s, 1H), 1.68 (d, J=15.0 Hz, 1H), 1.60-1.52 (m, 1H), 1.45 (dtd, J=15.8, 7.9, 6.4, 3.6 Hz, 1H), 1.42-1.30 (m, 1H), 1.31 (t, J=4.5 Hz, 1H), 1.27 (dd, J=9.6, 5.0 Hz, 1H), 0.95 (ddd, J=10.2, 6.6, 3.7 Hz, 1H), 0.87 (d, J=6.8 Hz, 3H), 0.82 (ddd, J=9.6, 6.6, 3.5 Hz, 1H).

Compound 18C*: LCMS (ESI): RT=0.74 min, m/z=409.1 [M+H]⁺; 1H NMR (400 MHz, DMSO-d6) δ 14.64 (s, 1H), 10.22 (s, 1H), 6.91 (d, J=8.6 Hz, 1H), 6.84 (dd, J=8.4, 6.6 Hz, 1H), 6.58 (d, J=8.1 Hz, 1H), 4.16 (q, J=6.9, 6.5 Hz, 1H), 3.61-3.47 (m, 3H), 3.18 (d, J=13.9 Hz, 1H), 1.76 (d, J=8.0 Hz, 1H), 1.67 (d, J=14.6 Hz, 1H), 1.61-1.21 (m, 5H), 0.95 (d, J=10.1 Hz, 1H), 0.90-0.77 (m, 4H).

Step 18: The assumed cis mixture product was purified by Prep-Chiral HPLC (CHIRAL ART Cellulose-SB, 2*25 cm, 5 μm; Mobile Phase A: Hexanes (0.1% trifluoroacetic acid (TFA)), Mobile Phase B: ethanol; Flow rate: 20 mL/min; Gradient: 8% B to 8% B in 18 min; Wave Length: 220/254 nm) to provide Compound 18A* (RT(min): 15.63, 4.4 mg) and Compound 18D* (RT(min): 17.71, 4.9 mg). Stereochemistry was arbitrarily assigned.

Compound 18A*: LCMS (ESI): RT=0.83 min, m/z=409 [M+H]⁺. ¹H NMR (400 MHz, DMSO-d6) δ 14.63 (s, 1H), 10.13 (s, 1H), 7.12 (d, J=8.3 Hz, 1H), 7.01 (d, J=8.5 Hz, 1H), 6.98-6.90 (m, 1H), 3.97 (s, 1H), 3.61-3.38 (m, 4H), 1.74 (d, J=13.5 Hz, 1H), 1.52 (d, J=12.2 Hz, 1H), 1.38 (s, 2H), 1.31 (s, 2H), 1.30-1.23 (m, 1H), 0.88 (d, J=6.6 Hz, 5H).

Compound 18D*: LCMS (ESI): RT=0.74 min, m/z=409 [M+H]⁺. ¹H NMR (400 MHz, DMSO-d6) δ 14.64 (s, 1H), 10.22 (s, 1H), 6.91 (d, J=8.6 Hz, 1H), 6.84 (dd, J=8.4, 6.6 Hz, 1H), 6.58 (d, J=8.1 Hz, 1H), 4.16 (q, J=6.9, 6.5 Hz, 1H), 3.61-3.47 (m, 3H), 3.18 (ddd, J=13.9, 9.7, 4.4 Hz, 1H), 1.76 (d, J=8.0 Hz, 1H), 1.67 (d, J=14.6 Hz, 1H), 1.61-1.21 (m, 5H), 0.95 (ddd, J=10.1, 6.6, 3.7 Hz, 1H), 0.90-0.77 (m, 4H).

Compounds 18A-OMe, 18B—OMe, 18C—OMe, and 18D-OMe may be separated by chiral HPLC from rac-18-OMe of step 14.

### Example 19. Synthesis of 1-(4-(difluoromethoxy)-2-methoxyphenyl)-N-((3R,5S)-5-methyl-1-(1H-tetrazol-5-yl)piperidin-3-yl)cyclopropane-1-carboxamide (Compound 19A-OMe), 1-(4-(difluoromethoxy)-2-hydroxyphenyl)-N-((3R,5S)-5-methyl-1-(1H-tetrazol-5-yl)piperidin-3-yl)cyclopropane-1-carboxamide (Compound 19A), and Compounds 19B, 19C, 19D, 19B—OMe, 19C—OMe, and 19D-OMe

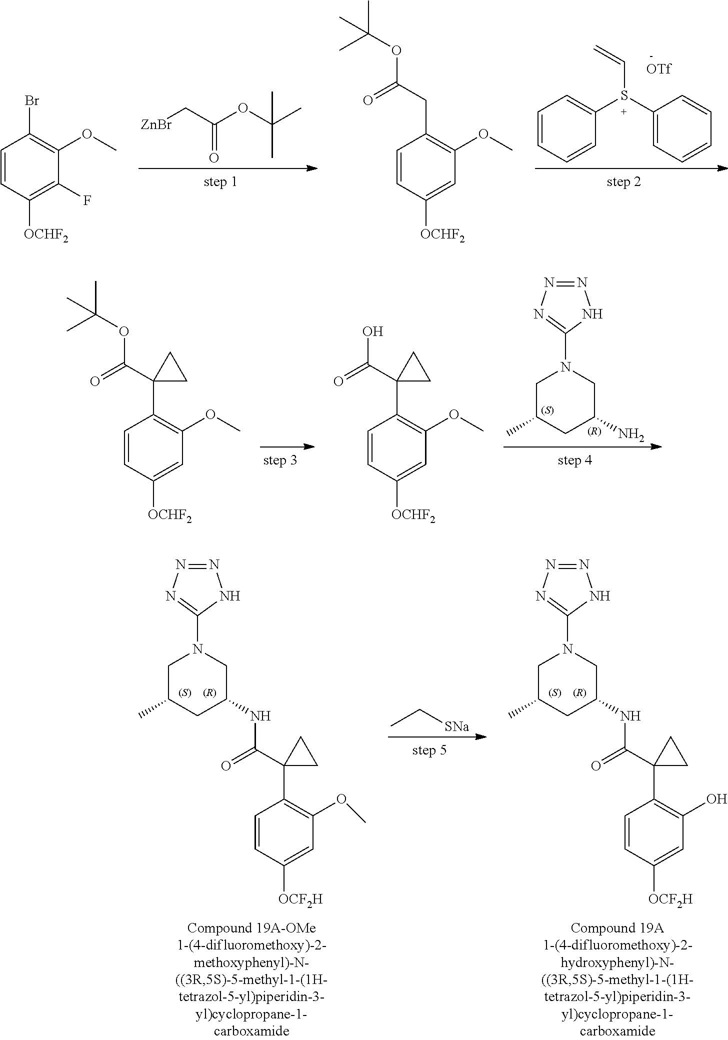

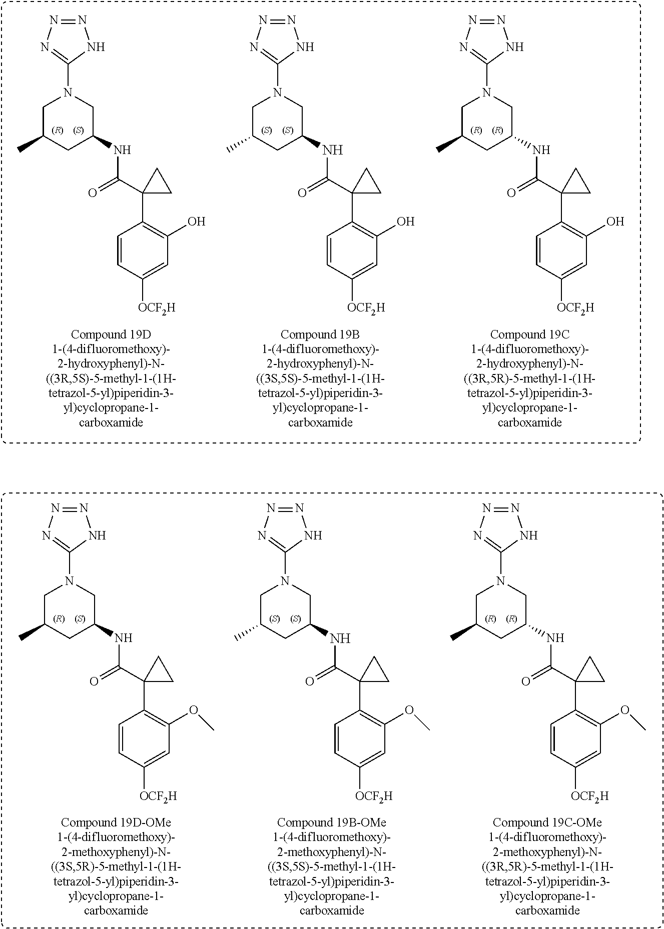

### Example 19 Follows Protocol A

Step 1: Into a 20 mL sealed tube was added 1-bromo-4-(difluoromethoxy)-2-methoxybenzene (600 mg, 2.371 mmol, 1 equiv), tert-butyl 2-(bromozincio)acetate (1852.62 mg, 7.113 mmol, 3 equiv), bis(tri-tert-butylphosphine)palladium(0) (Pd[(t-Bu)₃P]₂) (242.36 mg, 0.474 mmol, 0.2 equiv) and tetrahydrofuran (5 mL) at 60° C. The resulting mixture was stirred for 2 h at 60° C. under nitrogen atmosphere. The reaction was monitored by LCMS. The resulting mixture was extracted with ethyl acetate (EtOAc) (2×10 mL). The combined organic layers were washed with water (4×10 mL), dried over anhydrous Na₂SO₄. After filtration, the filtrate was concentrated under reduced pressure. The residue was purified by Prep-TLC (dichloromethane (DCM)/petroleum ether 1:1) to provide tert-butyl 2-[4-(difluoromethoxy)-2-methoxyphenyl]acetate (600 mg, 88% yield). LCMS (ES, m/z): RT=1.03 min, m/z=289.2[M+1]⁺.

Step 2: Into a 20 mL sealed tube were added tert-butyl 2-(4-(difluoromethoxy)-2-methoxyphenyl)acetate (600 mg, 2.08 mmol, 1 equiv), diphenyl(vinyl)sulfonium trifluoromethanesulfonate (2.26 g, 6.24 mmol, 3 equiv), 1,8-diazabicyclo[5.4.0]undec-7-one (DBU) (379 mg, 2.49 mmol, 1.2 equiv) and DMSO (5 mL) at room temperature. The resulting mixture was stirred for 2 h at 60° C. under nitrogen atmosphere. The reaction was monitored by LCMS. The resulting mixture was diluted with water (50 mL). The resulting mixture was extracted with EtOAc (2×200 mL). The combined organic layers were washed with brine (2×100 mL), dried over anhydrous Na₂SO₄. After filtration, the filtrate was concentrated under reduced pressure. The residue was purified by Prep-TLC (CH₂Cl₂/petroleum ether=1:1) to provide tert-butyl 2-[4-(difluoromethoxy)-2-methoxyphenyl]acetate (400 mg, 59% yield) as a light yellow oil. LCMS (ES, m/z): RT=1.02 min, m/z=314.2[M+H]⁺.

Step 3: Into an 8 mL vial was added tert-butyl 1-[4-(difluoromethoxy)-2-methoxyphenyl]cyclopropane-1-carboxylate (200 mg, 0.636 mmol, 1 equiv), dichloromethane (DCM) (2 mL) and trifluoroacetaldehyde (2 mL) at room temperature. The resulting mixture was stirred for 1 h at room temperature. The reaction was monitored by LCMS. The resulting mixture was concentrated under reduced pressure. The resulting mixture was extracted with ethyl acetate (EtOAc) (2×20 mL). The combined organic layers were washed with water (4×5 mL), dried over anhydrous Na₂SO₄. After filtration, the filtrate was concentrated under reduced pressure. This resulted in 1-[4-(difluoromethoxy)-2-methoxyphenyl]cyclopropane-1-carboxylic acid (150 mg, 91% yield). LCMS (ES, m/z): RT=1.12 min, m/z=257.0[M−1]⁻.

Step 4: Into a 8 L vial was added 1-[4-(difluoromethoxy)-2-methoxyphenyl]cyclopropane-1-carboxylic acid (“carboxyl (ii) reagent”) (50 mg, 0.194 mmol, 1 equiv), (3R,5S)-5-methyl-1-(1H-1,2,3,4-tetrazol-5-yl)piperidin-3-amine (“amine (i) reagent”) (70.5 mg, 0.388 mmol, 2 equiv), N,N,N′-tetramethylchloroformanidinium hexafluorophosphate (TCFH) (65.20 mg, 0.233 mmol, 1.2 equiv), N-methyl imidazole (NMI) (47.70 mg, 0.582 mmol, 3 equiv) and acetonitrile (MeCN) (0.5 mL, 0.019 mmol) at room temperature. The resulting mixture was stirred for 1 h at room temperature. The reaction was monitored by LCMS. The residue was purified by reverse flash chromatography (C18 silica gel; mobile phase, acetonitrile in water, 0% to 100% gradient in 30 min; detector, UV 254 nm) to provide 1-(4-(difluoromethoxy)-2-methoxyphenyl)-N-((3R,5S)-5-methyl-1-(1H-tetrazol-5-yl)piperidin-3-yl)cyclopropane-1-carboxamide (Compound 19A-OMe) (50 mg, 65% yield). LCMS (ES, m/z): RT=0.83 min, m/z=423.0[M−1]⁻.

Step 5: Into an 8 mL vial was added Compound 19A-OMe (30 mg, 0.071 mmol, 1 equiv), (ethylsulfanyl)sodium (89.60 mg, 1.065 mmol, 15 equiv) and dimethylformamide (DMF) (1 mL) at 120° C. The resulting mixture was stirred for 2 h at 80° C. The reaction was monitored by LCMS. The resulting mixture was filtered, the filter cake was washed with methanol (MeOH) (3×10 mL). The filtrate was concentrated under reduced pressure. The residue was purified by reverse flash chromatography (C18 silica gel; mobile phase, acetonitrile in water, 0% to 100% gradient in 10 min; detector, UV 254 nm) to provide crude product (30 mg), which was further purified by PREP HPLC (XBridge Prep Phenyl OBD Column, 19*250 mm, 5 μm; Mobile Phase A: water (0.05% trifluoroacetic acid (TFA)), Mobile Phase B: acetonitrile (MeCN); Flow rate: 25 mL/min; Gradient: 40% B to 50% B in 10 min, 50% B; Wave Length: 254 nm; RT(min): 9) to provide 1-(4-(difluoromethoxy)-2-hydroxyphenyl)-N-((3R,5S)-5-methyl-1-(1H-tetrazol-5-yl)piperidin-3-yl)cyclopropane-1-carboxamide (Compound 19A) (10.7 mg). LCMS (ES, m/z): RT=0.69 min, m/z=409.2[M+1]⁺. ¹H NMR (400 MHz, Methanol-d₄) δ 7.29-7.21 (m, 1H), 7.00-6.57 (m, 3H), 3.97-3.69 (m, 3H), 2.76-2.65 (m, 1H), 2.56 (m, J=12.7, 11.3 Hz, 1H), 1.96-1.75 (m, 2H), 1.51 (q, J=3.3, 2.9 Hz, 2H), 1.14-0.90 (m, 6H).

Compound 19B—OMe and Compound 19B may be synthesized from (3S,5S)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 4 of Example 19.

Compound 19C—OMe and Compound 19C may be synthesized from (3R,5R)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 4 of Example 19.

Compound 19D-OMe and Compound 19D may be synthesized from (3S,5R)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 4 of Example 19.

### Example 20. Synthesis 1-(2-methoxy-4-methylphenyl)-N-((3R,5S)-5-methyl-1-(1H-tetrazol-5-yl)piperidin-3-yl)cyclopropane-1-carboxamide (Compound 20A-OMe), 1-(2-hydroxy-4-methylphenyl)-N-((3R,5S)-5-methyl-1-(1H-tetrazol-5-yl)piperidin-3-yl)cyclopropane-1-carboxamide (Compound 20A), and Compounds 20B, 20C, 20D, 20B—OMe, 20C—OMe, and 20D-OMe

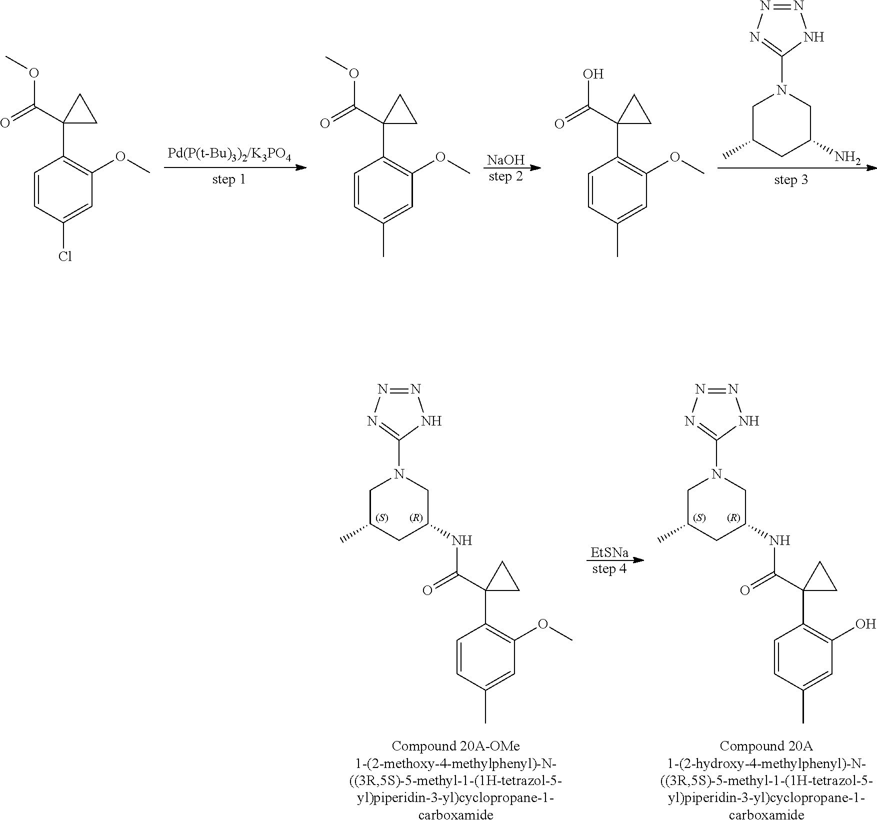

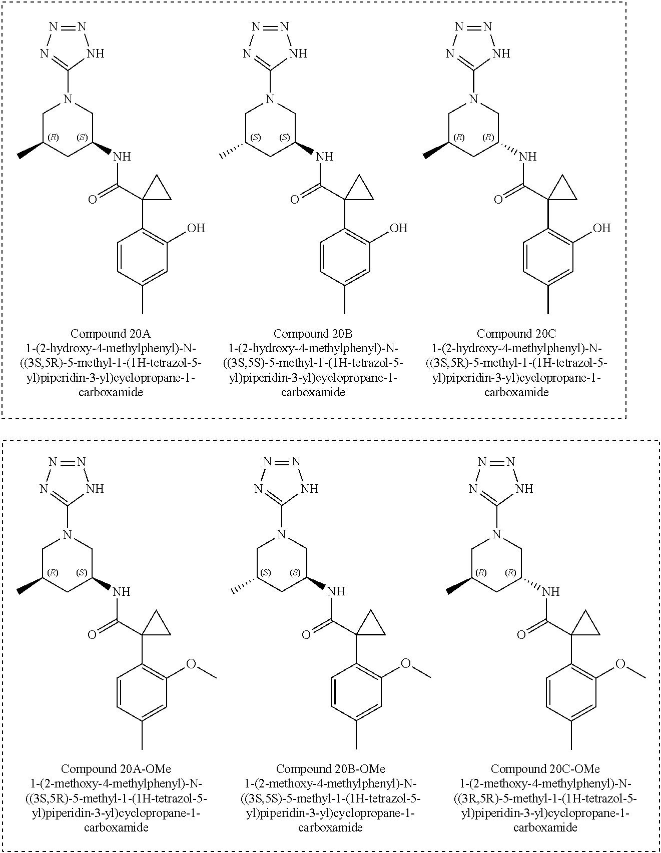

### Example 20 Follows Protocol A

Step 1: Into an 8 mL vial was added methyl 1-(4-chloro-2-methoxyphenyl)cyclopropane-1-carboxylate (220 mg, 0.914 mmol, 1 equiv), trimethyl-1,3,5,2,4,6-trioxatriborinane (137.69 mg, 1.097 mmol, 1.2 equiv), K₃PO₄ (582.08 mg, 2.742 mmol, 3 equiv), bis(tri-tert-butylphosphine)palladium(0) (Pd[(t-Bu)₃P]₂) (140.14 mg, 0.274 mmol, 0.3 equiv) and dioxane (4 mL). The resulting mixture was stirred for 2 h at 80° C. under nitrogen atmosphere. Desired product could be detected by LCMS. The resulting mixture was filtered, the filter cake was washed with ethyl acetate (EtOAc) (3×5 mL). The filtrate was concentrated under reduced pressure. The residue was purified by Prep-TLC (petroleum ether:ethyl acetate 8:1) to provide methyl 1-(2-methoxy-4-methylphenyl)cyclopropane-1-carboxylate (159 mg, 79% yield).

Step 2: Into an 8 mL vial was added methyl 1-(2-methoxy-4-methylphenyl)cyclopropane-1-carboxylate (140 mg, 0.636 mmol, 1 equiv), NaOH (76.27 mg, 1.908 mmol, 3 equiv), methanol (MeOH) (3 mL) and water (0.6 mL). The resulting mixture was stirred overnight at 40° C. under air atmosphere.

Desired product could be detected by LCMS. The mixture was acidified to pH 2 with conc. HCl. The aqueous layer was extracted with ethyl acetate (EtOAc) (3×10 mL), the resulting mixture was washed with brine (10 mL), dried over sodium sulfate, filtered and the solvent was removed under reduced pressure. The residue was purified by Prep-TLC (petroleum ether:ethyl acetate 2:1) to provide 1-(2-methoxy-4-methylphenyl)cyclopropane-1-carboxylic acid (106 mg, 81% yield).

Step 3: Into an 8 mL vial was added 1-(2-methoxy-4-methylphenyl)cyclopropane-1-carboxylic acid (“carboxyl (ii) reagent”) (100 mg, 0.485 mmol, 1 equiv), (3R,5S)-5-methyl-1-(1H-1,2,3,4-tetrazol-5-yl)piperidin-3-amine (“amine (i) reagent”) (106.03 mg, 0.582 mmol, 1.2 equiv), (1-[bis(dimethylamino)methylene]-1H-1,2,3-triazolo[4,5-b]pyridinium 3-oxide hexaflurophosphate (HATU) (184.36 mg, 0.485 mmol, 1 equiv), triethylamine (TEA) (147.20 mg, 1.455 mmol, 3 equiv) and dimethyl formamide (DMF) (2.5 mL). The resulting mixture was stirred for 2 h at room temperature under air atmosphere. Desired product could be detected by LCMS. The aqueous layer was extracted with ethyl acetate (EtOAc) (3×10 mL). The resulting mixture was washed with brine (10 mL), dried over sodium sulfate, filtered and the solvent was removed under reduced pressure. The residue was purified by Prep-TLC (Dichloromethane (DCM)/methanol (MeOH) 20:1) to provide 1-(2-methoxy-4-methylphenyl)-N-((3R,5S)-5-methyl-1-(1H-tetrazol-5-yl)piperidin-3-yl)cyclopropane-1-carboxamide (Compound 20A-OMe) (200 mg, 89% yield).

Step 4: Into an 8 mL vial was added Compound 20A-OMe (170 mg, 0.459 mmol, 1 equiv), (ethylsulfanyl)sodium (385.97 mg, 4.590 mmol, 10 equiv) and dimethyl formamide (DMF) (3.5 mL). The resulting mixture was stirred for 2 h at 120° C. under air atmosphere. Desired product could be detected by LCMS. The resulting mixture was filtered, the filter cake was washed with dimethyl formamide (DMF) (3×3 mL). The filtrate was concentrated under reduced pressure. The crude product was purified by PREP HPLC (XBridge Shield RP18 OBD Column, 30*150 mm, 5 μm; Mobile Phase A: water (10 mmol/L NH₄HCO₃), Mobile Phase B: acetonitrile (MeCN); Flow rate: 60 mL/min; Gradient: 19% B to 29% B in 10 min, 29% B; Wave Length: 254 nm; RT(min): 7.78) to provide 1-(2-hydroxy-4-methylphenyl)-N-((3R,5S)-5-methyl-1-(1H-tetrazol-5-yl)piperidin-3-yl)cyclopropane-1-carboxamide (Compound 20A) (29.6 mg). LCMS (ES, m/z): RT=0.67 min, m/z=357.2[M+H]⁺. ¹H NMR (400 MHz, DMSO-d₆) δ 7.05 (d, J=7.7 Hz, 1H), 6.67 (d, J=1.8 Hz, 1H), 6.61-6.57 (m, 1H), 6.13 (d, J=8.3 Hz, 1H), 3.86-3.63 (m, 3H), 2.34-2.24 (m, 1H), 2.22 (s, 3H), 2.20-2.10 (m, 1H), 1.74-1.59 (m, 2H), 1.38-1.26 (m, 2H), 0.93-0.77 (m, 6H).

Compound 20B—OMe and Compound 20B may be synthesized from (3S,5S)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 3 of Example 20.

Compound 20C—OMe and Compound 20C may be synthesized from (3R,5R)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 3 of Example 20.

Compound 20D-OMe and Compound 20D may be synthesized from (3S,5R)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 3 of Example 20.

### Example 21. Synthesis of 1-(4-chloro-2-hydroxy-3-methylphenyl)-N-(5-methyl-1-(1H-tetrazol-5-yl)azepan-3-yl)cyclopropane-1-carboxamide (Compound 21, rac-21), 1-(4-chloro-2-methoxy-3-methylphenyl)-N-(5-methyl-1-(1H-tetrazol-5-yl)azepan-3-yl)cyclopropane-1-carboxamide (Compound 21-OMe, rac-21-OMe), Compounds 21A*, 21B*, 21C*, and 21D*, and Compounds 21A-OMe*, 21B—OMe*, 21C—OMe*, and 21D-OMe*

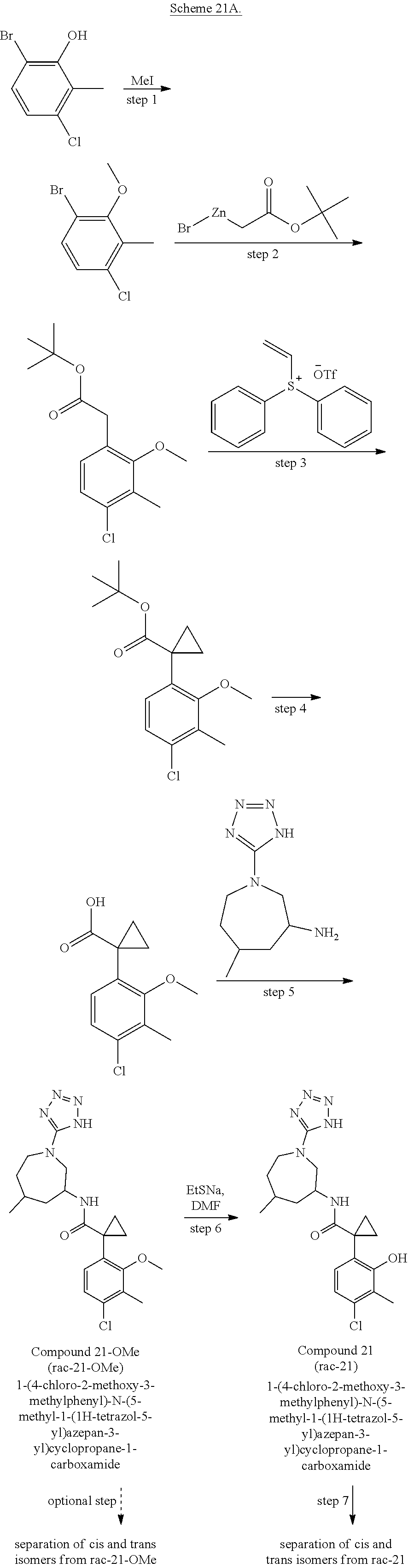

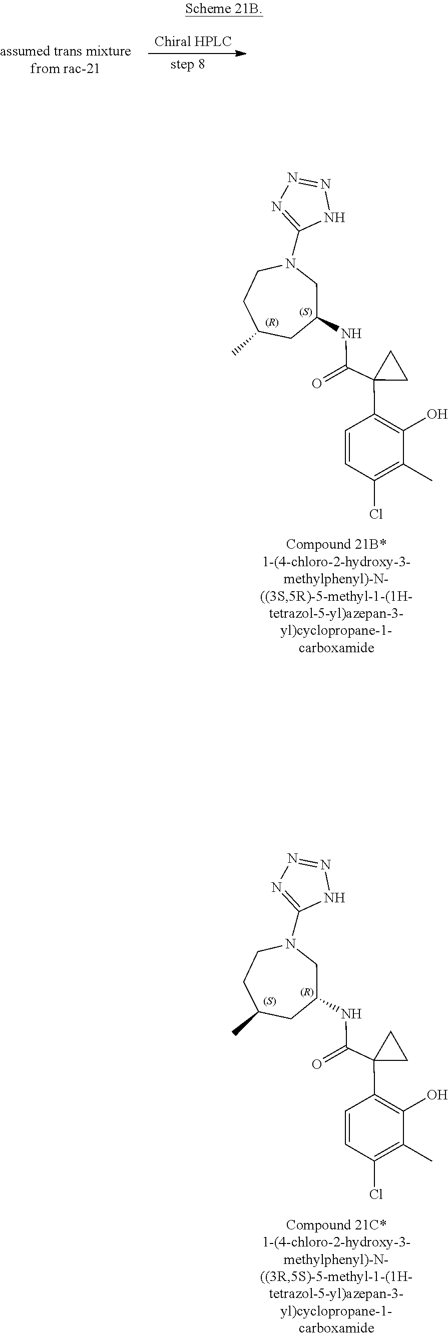

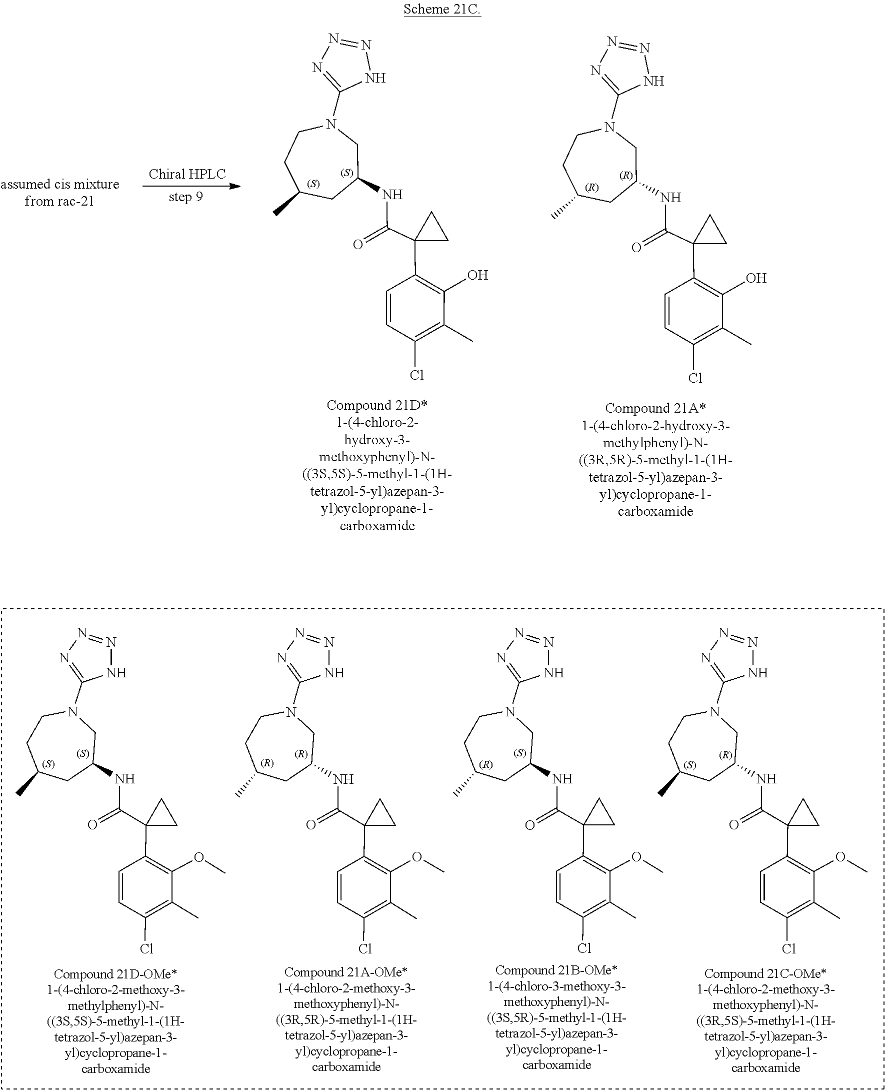

### Example 21 Follows Protocol A

Step 1: Into a 50 mL vial was added 6-bromo-3-chloro-2-methylphenol (1.00 g, 4.52 mmol, 1 equiv) and acetonitrile (MeCN) (10 mL), K₂CO₃ (1.87 g, 13.55 mmol, 3 equiv) and methyl iodide (1.28 g, 9.03 mmol, 2 equiv) at room temperature. The resulting mixture was stirred for 2 h at 80° C. under air atmosphere. Desired product could be detected by GCMS. The resulting mixture was extracted with ethyl acetate (EtOAc) (3×50 mL). The combined organic layers were washed with water (3×10 mL), dried over anhydrous Na₂SO₄. After filtration, the filtrate was concentrated under reduced pressure. The residue was purified by silica gel column chromatography, eluting with petroleum ether:ethyl acetate (5:1) to provide 1-bromo-4-chloro-2-methoxy-3-methylbenzene (800 mg, 68% yield). GCMS: (ES, m/z): RT=4.65 min, m/z=234.0.

Step 2: Into a 40 mL sealed tube was added 1-bromo-4-chloro-2-methoxy-3-methylbenzene (800 mg, 3.40 mmol, 1 equiv), tert-butyl 2-(bromozincio)acetate (2654.05 mg, 10.19 mmol, 3 equiv), tris(dibenzylideneacetone)dipalladium(0) (Pd₂(dba)₃) (622.13 mg, 0.68 mmol, 0.2 equiv) and tetrahydrofuran (THF) (16 mL) at room temperature. The resulting mixture was stirred for 2 h at 70° C. under nitrogen atmosphere. The reaction was monitored by TLC. The resulting mixture was extracted with ethyl acetate (EtOAc) (3×10 mL). The combined organic layers were washed with water (3×10 mL) and dried over anhydrous Na₂SO₄. After filtration, the filtrate was concentrated under reduced pressure to provide a residue, which was purified by Prep-TLC (petroleum ether:ethyl acetate 8:1; Rf=0.4) to provide tert-butyl 2-(4-chloro-2-methoxy-3-methylphenyl)acetate (400 mg, 39% yield).

Step 3: Into a 30 mL sealed tube was added tert-butyl 2-(4-chloro-2-methoxy-3-methylphenyl) acetate (400 mg, 1.48 mmol, 1 equiv), 1,8-diazabicyclo[5.4.0]undec-7-ene (DBU) (1.12 g, 7.38 mmol, 5 equiv), ethenyldiphenylsulfanium triflate (1.61 g, 4.43 mmol, 3 equiv) and dimethylsulfoxide (DMSO) (4 mL) at room temperature. The resulting mixture was stirred for 2 h at room temperature under nitrogen atmosphere. The reaction was monitored by GCMS. The resulting mixture was extracted with ethyl acetate (EtOAc) (3×10 mL). The combined organic layers were washed with water (3×10 mL), dried over anhydrous Na₂SO₄. After filtration, the filtrate was concentrated under reduced pressure to provide a residue, which was purified by reverse flash chromatography (C18 silica gel; mobile phase, acetonitrile (MeCN) in water, 10% to 50% gradient in 10 min; detector, UV 254 nm) to provide tert-butyl 1-(4-chloro-2-methoxy-3-methylphenyl)cyclopropane-1-carboxylate (220 mg, 50% yield). LCMS (ES, m/z): RT=0.87 min.

Step 4: Into a 20 mL vial was added tert-butyl 1-(4-chloro-2-methoxy-3-methylphenyl)cyclopropane-1-carboxylate (220 mg, 0.74 mmol, 1 equiv), dichloromethane (DCM) (2 mL) and trifluoroacetic acid (TFA) (0.4 mL) at room temperature. The resulting mixture was stirred for 1h at room temperature under air atmosphere. The reaction was monitored by LCMS. Upon completion, the resulting mixture was concentrated under reduced pressure to provide 1-(4-chloro-2-methoxy-3-methylphenyl)cyclopropane-1-carboxylic acid (180 mg, 91% yield). LCMS (ES, m/z): RT=0.67 min, m/z=241.0[M+1]⁺.

Step 5: Into a 20 mL vial was added 1-(4-chloro-2-methoxy-3-methylphenyl)cyclopropane-1-carboxylic acid (“carboxyl (ii) reagent”) (170 mg, 0.71 mmol, 1 equiv), (1-[bis(dimethylamino)methylene]-1H-1,2,3-triazolo[4,5-b]pyridinium 3-oxide hexafluorophosphate (HATU) (295.43 mg, 0.78 mmol, 1.1 equiv), dimethyl formamide (DMF) (6 mL), triethylamine (TEA) (357.38 mg, 3.53 mmol, 5 equiv), and 5-methyl-1-(1H-1,2,3,4-tetrazol-5-yl)azepan-3-amine (“amine (i) reagent”) (277.25 mg, 1.412 mmol, 2 equiv) at room temperature. The resulting mixture was stirred for 2 h at room temperature under air atmosphere. The reaction was monitored by LCMS. Upon completion, the residue was purified by reverse flash chromatography (C18 silica gel; mobile phase, acetonitrile (MeCN) in water, 10% to 40% gradient in 20 min; detector, UV 254 nm) to provide 1-(4-chloro-2-methoxy-3-methylphenyl)-N-(5-methyl-1-(1H-tetrazol-5-yl)azepan-3-yl)cyclopropane-1-carboxamide (Compound 21-OMe, rac-21-OMe) (160 mg, 49% yield). LCMS (ES, m/z): RT=0.84 min, m/z=419.0[M+1]⁺.

Step 6: Into a 40 mL vial was added rac-21-OMe (150 mg, 0.36 mmol, 1 equiv), dimethylsulfoxide (DMSO) (10 mL), and (ethylsulfanyl)sodium (451.77 mg, 5.37 mmol, 15 equiv) at room temperature. The resulting mixture was stirred for 2 h at 120° C. under air atmosphere. The reaction was monitored by LCMS. Upon completion, the reaction was concentrated, and the residue was purified by reverse flash chromatography (C18 silica gel; mobile phase, acetonitrile (MeCN) in water, 30% to 50% gradient in 10 min; detector, UV 254 nm) to provide 1-(4-chloro-2-hydroxy-3-methylphenyl)-N-(5-methyl-1-(1H-tetrazol-5-yl)azepan-3-yl)cyclopropane-1-carboxamide (Compound 21, rac-21) (30 mg, 19% yield) as a mixture of cis and trans isomers. LCMS (ES, m/z): RT=1.30 min, m/z=405.0[M+1]⁺.

Step 7: The crude product, rac-21, (30 mg) was purified by PREP HPLC (XBridge Prep OBD C18 Column, 30*150 mm, 5 μm; Mobile Phase A: Water (10 mmol/L NH₄HCO₃), Mobile Phase B: acetonitrile; Flow rate: 60 mL/min; Gradient: 22% B to 32% B in 8 min; Wave Length: 254 nm; RT(min): 7-10.5) to provide the assumed cis mixture (30 mg, 19% yield) as a first eluting peak and the assumed trans mixture (30 mg, 19% yield) as a second eluting peak.

Step 8: The assumed trans mixture (30 mg) was separated by Chiral HPLC (Lux 5 μm Cellulose-4, 2.12*25 cm, 5 μm; Mobile Phase A: hexanes (0.1% trifluoroacetic acid (TFA)), Mobile Phase B: isopropanol; Flow rate: 20 mL/min; Gradient: 30% B to 30% B in 19 min; Wave Length: 220/254 nm) to provide Compound 21B* (RT(min): 10.75, 10.1 mg) and Compound 21C* (RT(min): 15.88, 14.9 mg). Stereochemistry arbitrarily assigned.

Compound 21B*: LCMS (ES, m/z): RT=1.30 min, m/z=405.1[M+1]⁺. ¹H NMR (400 MHz, DMSO-d₆) δ 14.60 (s, 1H), 9.59 (s, 1H), 6.97-6.87 (m, 2H), 6.80 (d, J=8.3 Hz, 1H), 4.21-4.10 (m, 1H), 3.64-3.46 (m, 3H), 3.22-3.11 (m, 1H), 2.20 (s, 3H), 1.81-1.72 (m, 1H), 1.67 (d, J=14.5 Hz, 1H), 1.59-1.41 (m, 2H), 1.40-1.34 (m, 1H), 1.31-1.21 (m, 2H), 0.97-0.89 (m, 1H), 0.87 (d, J=6.8 Hz, 3H), 0.83-0.75 (m, 1H).

Compound 21C*: LCMS (ES, m/z): RT=1.43 min, m/z=405.1[M+1]⁺. ¹H NMR (400 MHz, DMSO-d₆) δ 14.60 (s, 1H), 9.59 (s, 1H), 6.98-6.87 (m, 2H), 6.80 (d, J=8.2 Hz, 1H), 4.21-4.08 (m, 1H), 3.63-3.46 (m, 3H), 3.22-3.10 (m, 1H), 2.20 (s, 3H), 1.83-1.71 (m, 1H), 1.68 (d, J=14.3 Hz, 1H), 1.60-1.41 (m, 2H), 1.41-1.32 (m, 1H), 1.32-1.21 (m, 2H), 0.96-0.90 (m, 1H), 0.87 (d, J=6.8 Hz, 3H), 0.83-0.76 (m, 1H).

Step 9: The assumed cis mixture (30 mg) was separated by Chiral HPLC (Lux 5 μm Cellulose-4, 2.12*25 cm, 5 μm; Mobile Phase A: Hexanes (0.1% trifluoroacetic acid (TFA)), Mobile Phase B: isopropanol; Flow rate: 20 mL/min; Gradient: 20% B to 20% B in 28 min; Wave Length: 220/254 nm) to provide Compound 21D* (RT(min): 20.08, 4.2 mg) and Compound 21A* (RT(min): 25.04, 2.2 mg). Stereochemistry arbitrarily assigned.

Compound 21A*: LCMS (ES, m/z): RT=1.513 min, m/z=405.1[M+1]⁺. ¹H NMR (400 MHz, DMSO-d₆) δ 7.71 (s, 1H), 7.01 (d, J=8.2 Hz, 1H), 6.88 (d, J=8.3 Hz, 1H), 3.93 (d, J=8.6 Hz, 1H), 3.63-3.52 (m, 2H), 3.47-3.40 (m, 1H), 3.26-3.20 (m, 1H), 2.24 (s, 2H), 1.74-1.64 (m, 1H), 1.58-1.46 (m, 2H), 1.43-1.28 (m, 3H), 1.19-1.06 (m, 1H), 0.88 (t, J=7.7 Hz, 5H).

Compound 21D*: LCMS (ES, m/z): RT=1.503 min, m/z=405.1[M+1]⁺. ¹H NMR (400 MHz, DMSO-d₆) δ 7.96 (s, 1H), 7.00 (d, J=8.2 Hz, 1H), 6.87 (d, J=8.3 Hz, 1H), 3.99-3.90 (m, 1H), 3.70-3.64 (m, 1H), 3.56-3.43 (m, 2H), 3.20-3.11 (m, 1H), 2.25 (s, 3H), 1.70-1.60 (m, 1H), 1.58-1.46 (m, 2H), 1.44-1.29 (m, 3H), 1.14-1.01 (m, 1H), 0.95-0.89 (m, 1H), 0.86 (d, J=6.7 Hz, 4H).

Compounds 21A-OMe, 21B—OMe, 21C—OMe, and 21D-OMe may be separated by chiral HPLC from rac-21-OMe of step 5.

### Example 22. Synthesis of 1-(3-fluoro-2-hydroxy-4-methylphenyl)-N-(5-methyl-1-(1H-tetrazol-5-yl)azepan-3-yl)cyclopropane-1-carboxamide (Compound 22, rac-22), 1-(3-fluoro-2-methoxy-4-methylphenyl)-N-(5-methyl-1-(1H-tetrazol-5-yl)azepan-3-yl)cyclopropane-1-carboxamide (Compound 22-OMe, rac-22-OMe), Compounds 22A*, 22B*, 22C*, and 22D*, and Compounds 22A-OMe*, 22B—OMe*, 22C—OMe*, and 22D-OMe*

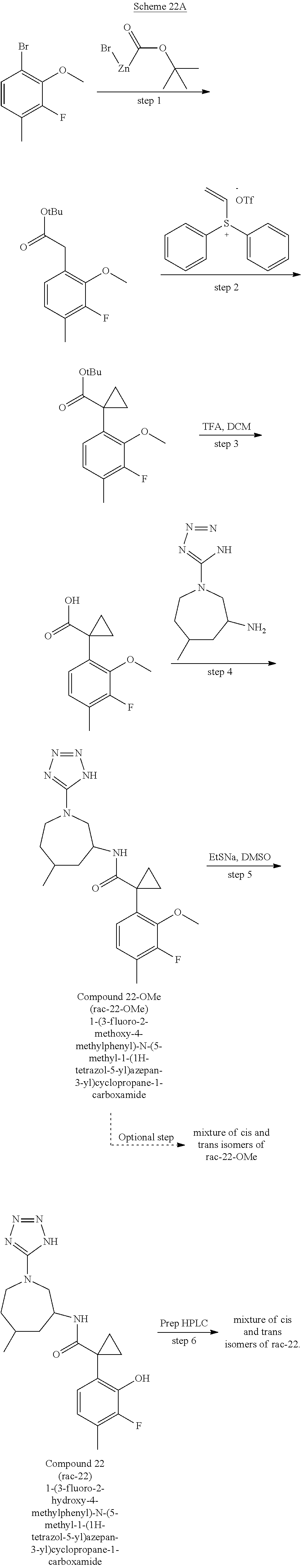

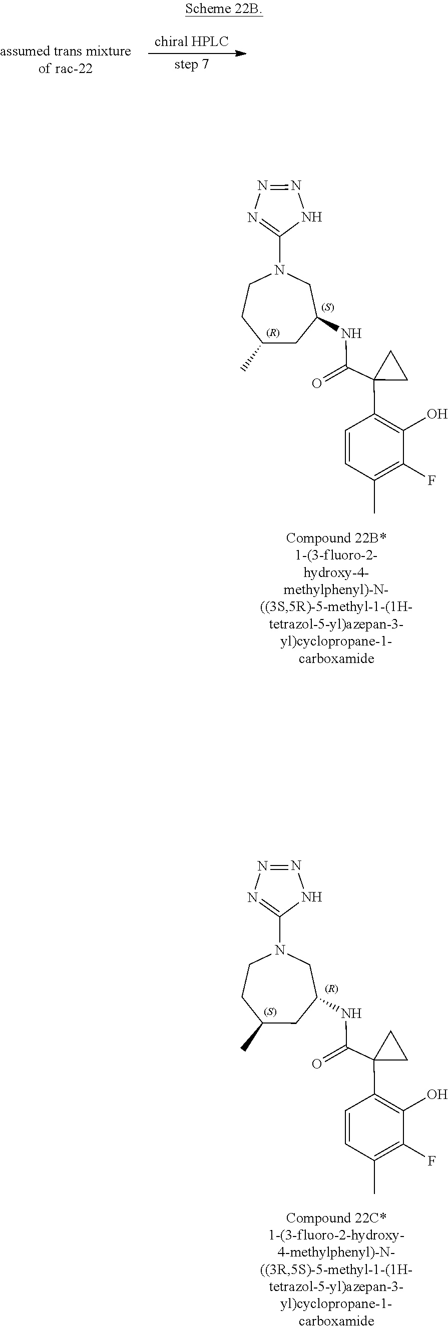

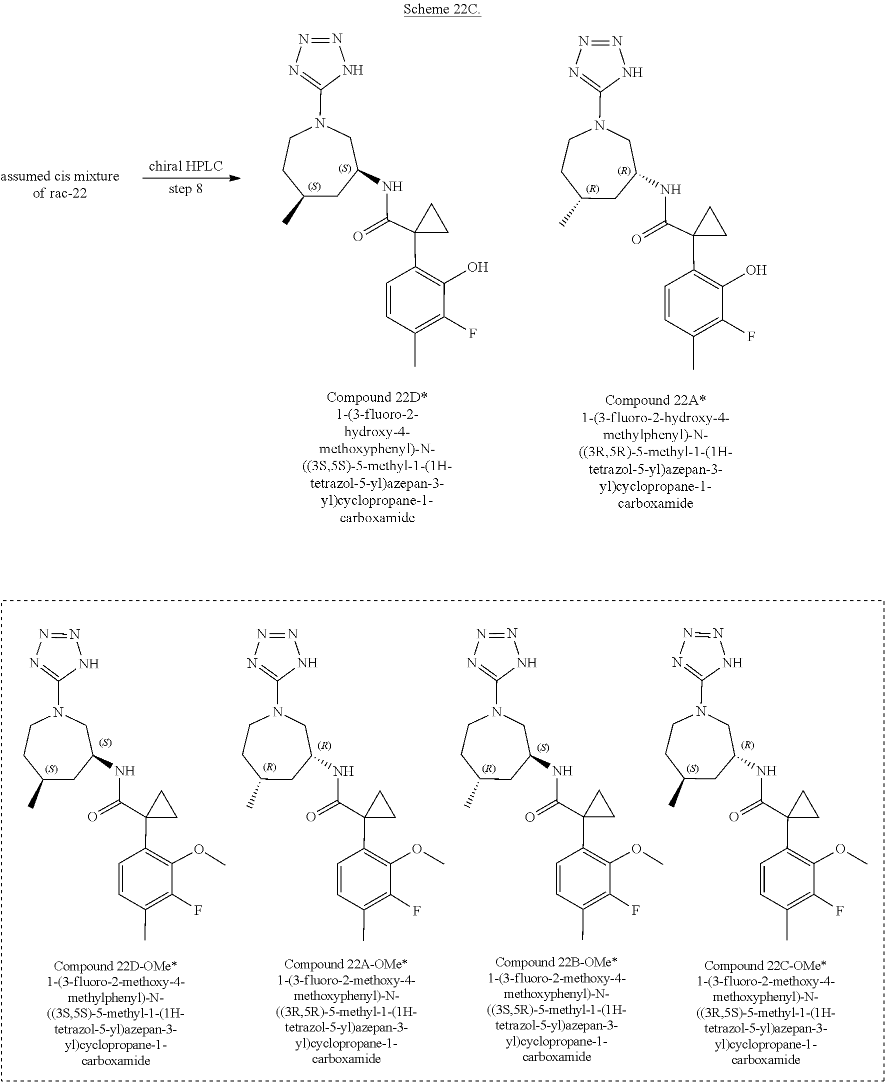

### Example 22 Follows Protocol A

Step 1: A solution of 1-bromo-3-fluoro-2-methoxy-4-methylbenzene (1000 mg, 4.56 mmol, 1 equiv) and 1-(bromozincio)-3-(tert-butoxy)propan-2-one (3758.82 mg, 13.69 mmol, 3 equiv), bis(tri-tert-butylphosphine)palladium(0) (Pd[(t-Bu)₃P]₂) (699.91 mg, 1.370 mmol, 0.3 equiv) in tetrahydrofuran (THF) (80 mL) was stirred for 2 h at 60° C. under nitrogen atmosphere. The resulting mixture was extracted with ethyl acetate (EtOAc) (3×20 mL). The combined organic layers were washed with water (3×10 mL), dried over anhydrous Na₂SO₄. After filtration, the filtrate was concentrated under reduced pressure. The residue was purified by reversed-phase flash chromatography (C18 silica gel; mobile phase, acetonitrile (MeCN) in water, 50% to 65% gradient in 10 min; detector, UV 220 nm) to provide tert-butyl 2-(3-fluoro-2-methoxy-4-methylphenyl)acetate (800 mg, 69% yield). LCMS (ES, m/z): RT=1.103 min, m/z=153.15[M+H]⁺.

Step 2: A solution of tert-butyl 2-(3-fluoro-2-methoxy-4-methylphenyl)acetate (400 mg, 1.57 mmol, 1 equiv) and ethenyldiphenylsulfanium (671.08 mg, 3.14 mmol, 2 equiv), 1,8-diazabicyclo[5.4.0]undec-7-ene (DBU) (718.40 mg, 4.71 mmol, 3 equiv), triflate (468.92 mg, 3.14 mmol, 2 equiv) in dimethylsulfoxide (DMSO) (20 mL) was stirred for 2 h at room temperature. The resulting mixture was extracted with ethyl acetate (EtOAc) (3×20 mL). The combined organic layers were washed with water (3×10 mL), dried over anhydrous Na₂SO₄. After filtration, the filtrate was concentrated under reduced pressure. The residue was purified by reversed-phase flash chromatography (C18 silica gel; mobile phase, acetonitrile (MeCN) in water, 55% to 65% gradient in 10 min; detector, UV 220 nm) to provide tert-butyl 1-(3-fluoro-2-methoxy-4-methylphenyl)cyclopropane-1-carboxylate. LCMS (ES, m/z): RT=1.145 min, m/z=179.05[M+H]⁺.

Step 3: A solution of tert-butyl 1-(3-fluoro-2-methoxy-4-methylphenyl)cyclopropane-1-carboxylate (260 mg, 0.92 mmol, 1 equiv) and trifluoroacetic acid (TFA) (10 mL) in dichloromethane (DCM) (50 mL) was stirred for 1 h at room temperature. The resulting mixture was concentrated under reduced pressure. The residue was purified by reversed-phase flash chromatography (C18 silica gel; mobile phase, acetonitrile (MeCN) in water, 35% to 45% gradient in 10 min; detector, UV 220 nm) to provide 1-(3-fluoro-2-methoxy-4-methylphenyl)cyclopropane-1-carboxylic acid (200 mg, 96% yield). LCMS (ES, m/z): RT=0.853 min, m/z=179.00[M+H]⁺.

Step 4: A solution of 1-(3-fluoro-2-methoxy-4-methylphenyl)cyclopropane-1-carboxylic acid (“carboxyl (ii) reagent”) (180 mg, 0.80 mmol, 1 equiv) and 3-methyl-6-(1H-1,2,3,4-tetrazol-5-yl)cycloheptan-1-amine (“amine (i) reagent”) (156.75 mg, 0.80 mmol, 1 equiv), (1-[bis(dimethylamino)methylene]-1H-1,2,3-triazolo[4,5-b]pyridinium 3-oxide hexafluorophosphate (HATU) (366.28 mg, 0.96 mmol, 1.2 equiv), triethylamine (TEA) (243.70 mg, 2.40 mmol, 3 equiv) in dimethyl formamide (DMF) (10 mL) was stirred for 1 h at room temperature. The resulting mixture was extracted with ethyl acetate (EtOAc) (3×10 mL). The combined organic layers were washed with water (3×10 mL), dried over anhydrous Na₂SO₄. After filtration, the filtrate was concentrated under reduced pressure. The residue was purified by reversed-phase flash chromatography (C18 silica gel; mobile phase, acetonitrile (MeCN) in water, 35% to 45% gradient in 10 min; detector, UV 220 nm) to provide 1-(3-fluoro-2-methoxy-4-methylphenyl)-N-(5-methyl-1-(1H-tetrazol-5-yl)azepan-3-yl)cyclopropane-1-carboxamide (Compound 22-OMe, rac-22-OMe) (100 mg, 31% yield) as a mixture of cis and trans isomers. LCMS (ES, m/z): RT=0.820 min, m/z=403.25[M+H]+.

Step 5: A solution of rac-22-OMe (100 mg, 0.24 mmol, 1 equiv) and (ethylsulfanyl)sodium (313.50 mg, 3.72 mmol, 15 equiv) in dimethylsulfoxide (DMSO) (5 mL) was stirred for 1.5h at 120° C. under nitrogen atmosphere. The residue was purified by reversed-phase flash chromatography (C18 silica gel; mobile phase, acetonitrile (MeCN) in water, 60% to 70% gradient in 10 min; detector, UV 220 nm) to provide the crude product, 1-(3-fluoro-2-hydroxy-4-methylphenyl)-N-(5-methyl-1-(1H-tetrazol-5-yl)azepan-3-yl)cyclopropane-1-carboxamide (Compound 22, rac-22) (3 mg, 3.08% yield), as a mixture of cis and trans isomers.

Step 6: The product, rac-22, was purified by PREP HPLC (XBridge Prep Phenyl OBD Column, 19*250 mm, 5 μm; Mobile Phase A: water(10 mmol/L NH₄HCO₃), Mobile Phase B: acetonitrile (MeCN); Flow rate: 20 mL/min; Gradient: 21% B to 23% B in 8 min, 23% B; Wave Length: 254 nm) to provide the assumed trans product (6.0 mg, 6% yield) as first eluting peak and the assumed cis mixture (9 mg, 9% yield) as second eluting peak, stereochemistry arbitrarily assigned.

Step 7: The assumed trans product (6 mg, 99% purity) was purified by Prep-Chiral HPLC (CHIRALPAK IE, 2*25 cm, 5 μm; Mobile Phase A: Hexanes (0.1% trifluoroacetic acid (TFA), Mobile Phase B: ethanol:dichloromethane=1:1; Flow rate: 20 mL/min; Gradient: 30% B to 30% B in 16 min; Wave Length: 220/254 nm) to provide Compound 22B* (RT(min): 9.86, 2.1 mg) and Compound 22C* (RT(min): 13.97, 2.2 mg). Stereochemistry arbitrarily assigned.

Compound 22B*: LCMS (ES, m/z): RT=0.695 min, m/z=389.20[M+H]+. ¹H NMR (400 MHz, Methanol-d4) δ 6.71 (dd, J=7.9, 1.5 Hz, 1H), 6.48 (t, J=7.5 Hz, 1H), 4.14 (d, J=5.0 Hz, 1H), 3.74 (dd, J=15.2, 5.7 Hz, 1H), 3.57 (dd, J=15.0, 2.0 Hz, 1H), 3.45 (dt, J=12.4, 4.3 Hz, 1H), 3.22-3.10 (m, 1H), 2.22 (d, J=2.2 Hz, 3H), 1.88 (d, J=14.2 Hz, 1H), 1.79 (dd, J=11.5, 6.1 Hz, 1H), 1.71-1.58 (m, 2H), 1.58-1.29 (m, 2H), 1.11-0.97 (m, 3H), 0.92 (ddd, J=9.8, 6.8, 3.6 Hz, 1H).

Compound 22C*: LCMS (ES, m/z): RT=0.687 min, m/z=389.20[M+H]+. ¹H NMR (400 MHz, Methanol-d4) δ 6.74-6.68 (m, 1H), 6.48 (t, J=7.5 Hz, 1H), 4.13 (s, 1H), 3.74 (dd, J=14.9, 5.8 Hz, 1H), 3.62-3.53 (m, 1H), 3.45 (dd, J=12.5, 4.6 Hz, 1H), 2.22 (d, J=2.2 Hz, 3H), 1.87 (d, J=14.2 Hz, 1H), 1.80 (d, J=13.0 Hz, 1H), 1.66 (dd, J=17.0, 7.1 Hz, 2H), 1.53 (ddd, J=10.1, 6.8, 3.5 Hz, 1H), 1.49-1.34 (m, 1H), 1.37-1.29 (m, 1H), 1.11-0.97 (m, 3H), 0.92 (ddd, J=9.6, 6.7, 3.6 Hz, 1H).

Step 8: The assumed cis product (9 mg, 99.1% purity) was purified by Prep-Chiral HPLC (Lux 5 μm Cellulose-4, 2.12*25 cm, 5 gm; Mobile Phase A: Hexanes (0.1% trifluoroacetic acid (TFA)), Mobile Phase B: isopropanol; Flow rate: 20 mL/min; Gradient: 30% B to 30% B in 21 min; Wave Length: 220\254 nm) to provide Compound 22D* (RT(min): 14.21, 3 mg) and Compound 22A* (RT(min): 18.03, 2.9 mg). Stereochemistry arbitrarily assigned.

Compound 22A*: LCMS (ES, m/z): RT=0.787 min, m/z=389.2[M+H]+. ¹H NMR (400 MHz, Methanol-d4) δ 6.92 (dd, J=7.9, 1.5 Hz, 1H), 6.70 (t, J=7.5 Hz, 1H), 3.98 (ddt, J=12.0, 8.4, 4.3 Hz, 1H), 3.68-3.51 (m, 2H), 3.51-3.40 (m, 1H), 2.28 (d, J=2.2 Hz, 3H), 1.85 (d, J=14.5 Hz, 1H), 1.67 (dd, J=27.4, 11.2 Hz, 2H), 1.60-1.42 (m, 3H), 1.33-1.17 (m, 1H), 1.10-0.98 (m, 1H), 0.98 (d, J=6.7 Hz, 3H).

Compound 22D*: LCMS (ES, m/z): RT=1.400 min, m/z=389.20[M+H]+. ¹H NMR (400 MHz, Methanol-d4) δ 6.92 (dd, J=7.9, 1.5 Hz, 1H), 6.70 (t, J=7.5 Hz, 1H), 3.98 (ddt, J=11.9, 8.4, 4.3 Hz, 1H), 3.65-3.40 (m, 4H), 2.28 (d, J=2.2 Hz, 3H), 1.84 (d, J=14.2 Hz, 1H), 1.70 (d, J=12.7 Hz, 1H), 1.60-1.42 (m, 2H), 1.24 (dt, J=13.4, 10.8 Hz, 1H), 1.10-0.98 (m, 2H), 0.98 (d, J=6.7 Hz, 3H).

Compounds 22A-OMe, 22B—OMe, 22C—OMe, and 22D-OMe may be separated by chiral HPLC from rac-22-OMe of step 4.

### Example 23. Synthesis of 1-(4-cyclopropyl-2-hydroxyphenyl)-N-((3R,5S)-5-methyl-1-(1H-tetrazol-5-yl)piperidin-3-yl)cyclopropane-1-carboxamide (Compound 23A), 1-(4-cyclopropyl-2-methoxyphenyl)-N-((3R,5S)-5-methyl-1-(1H-tetrazol-5-yl)piperidin-3-yl)cyclopropane-1-carboxamide (Compound 23A-OMe), and Compounds 23B, 23C, 23D, 23B—OMe, 23C—OMe, and 23D-OMe

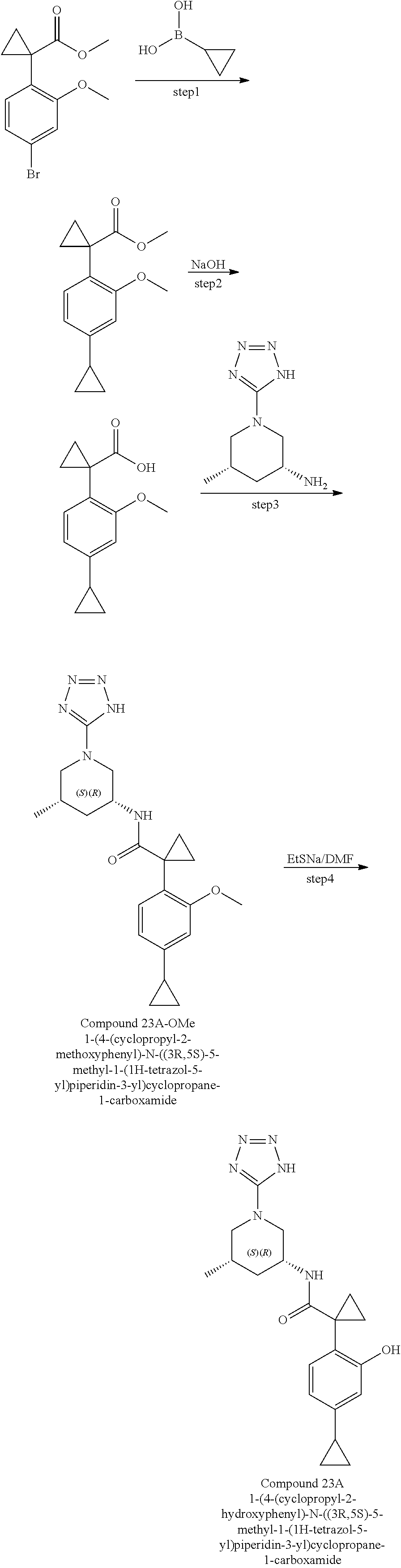

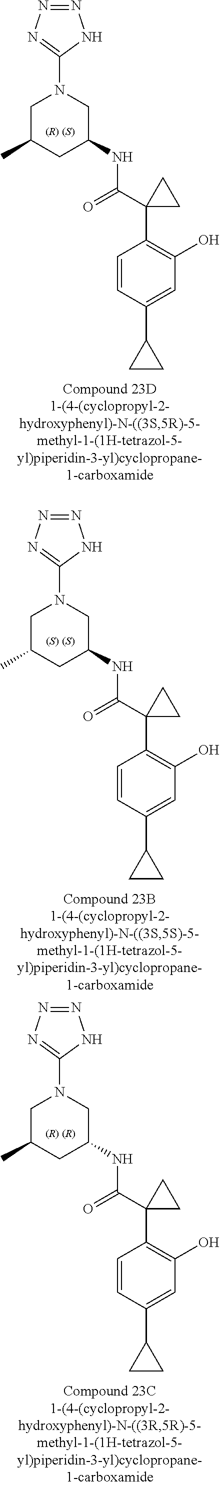

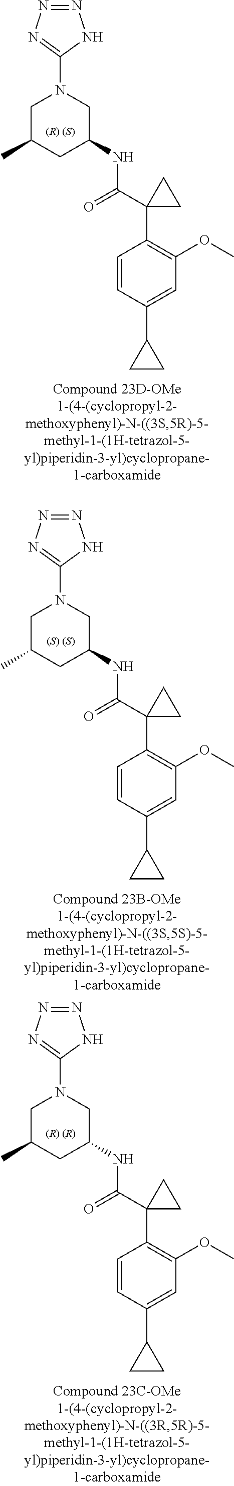

### Example 23 Follows Protocol A

Step 1: To a solution of methyl 1-(4-bromo-2-methoxyphenyl)cyclopropane-1-carboxylate (160 mg, 0.561 mmol, 1 equiv) and cyclopropylboronic acid (57.84 mg, 0.673 mmol, 1.2 equiv) in dioxane (3 mL) and water (0.6 mL) was added Na₂CO₃ (178.42 mg, 1.683 mmol, 3 equiv) and [1,1′-bis(diphenylphosphino)ferrocene]palladium(1I) dichloride (Pd(dppf)Cl₂) (123.18 mg, 0.168 mmol, 0.3 equiv). The resulting mixture was stirred for 2 h at 80° C. under nitrogen atmosphere. Desired product could be detected by LCMS. The resulting mixture was filtered, the filter cake was washed with ethyl acetate (EtOAc) (3×5 mL). The filtrate was concentrated under reduced pressure, and then purified by Prep-TLC (petroleum ether:ethyl acetate 5:1) to provide methyl 1-(4-cyclopropyl-2-methoxyphenyl)cyclopropane-1-carboxylate (75 mg, 54% yield).

Step 2: Into an 8 mL vial was added methyl 1-(4-cyclopropyl-2-methoxyphenyl)cyclopropane-1-carboxylate (70 mg, 0.284 mmol, 1 equiv), NaOH (34.10 mg, 0.852 mmol, 3 equiv), ethanol (MeOH) (3 mL) and water (0.6 mL). The resulting mixture was stirred for 48 h at 40° C. under air atmosphere.

Desired product could be detected by LCMS. The resulting mixture was concentrated under reduced pressure to provide an oil which was then acidified to pH 3 with conc. HCl. The aqueous layer was extracted with ethyl acetate (EtOAc) (3×10 mL). The combined organic layers were washed with brine (1×10 mL), dried over anhydrous Na₂SO₄, filtered, and the filtrate was concentrated under reduced pressure to provide a residue, to provide 1-(4-cyclopropyl-2-methoxyphenyl)cyclopropane-1-carboxylic acid (71 mg, 86% yield).

Step 3: Into an 8 mL vial was added 1-(4-cyclopropyl-2-methoxyphenyl)cyclopropane-1-carboxylic acid (“carboxyl (ii) reagent”) (70 mg, 0.301 mmol, 1 equiv), (3R,5S)-5-methyl-1-(1H-1,2,3,4-tetrazol-5-yl)piperidin-3-amine (“amine (i) reagent”) (65.90 mg, 0.361 mmol, 1.2 equiv), N,N,N′,N′-tetramethylchloroformamidinium hexafluorophosphate (TCFH) (101.47 mg, 0.361 mmol, 1.2 equiv), N-methyl imidazole (NMI) (74.23 mg, 0.903 mmol, 3 equiv) and acetonitrile (MeCN) (1.5 mL). The resulting mixture was stirred for 2 h at room temperature under air atmosphere. Desired product could be detected by LCMS. The resulting oil was dried under vacuum. The residue was purified by Prep-TLC (dichloromethane (DCM)/methanol (MeOH) 18:1) to provide 1-(4-cyclopropyl-2-methoxyphenyl)-N-((3R,5S)-5-methyl-1-(1H-tetrazol-5-yl)piperidin-3-yl)cyclopropane-1-carboxamide (Compound 23A-OMe) (150 mg, 75% yield).

Step 4: Into an 8 mL vial was added Compound 23A-OMe (85 mg, 0.214 mmol, 1 equiv), (ethylsulfanyl)sodium (270.49 mg, 3.210 mmol, 15 equiv) and dimethyl formamide (DMF) (2 mL). The resulting mixture was stirred overnight at 120° C. under air atmosphere. Desired product could be detected by LCMS. The resulting mixture was filtered, the filter cake was washed with ethyl acetate (EtOAc) (3×5 mL). The filtrate was concentrated under reduced pressure. The residue was purified by reverse flash chromatography (C18 silica gel; mobile phase, acetonitrile:water=1:6) to provide 1-(4-cyclopropyl-2-hydroxyphenyl)-N-((3R,5S)-5-methyl-1-(1H-tetrazol-5-yl)piperidin-3-yl)cyclopropane-1-carboxamide (Compound 23A), which was further purified by PREP HPLC (XBridge Prep Phenyl OBD Column, 19*250 mm, 5 μm; Mobile Phase A: water (0.05% trifluoroacetic acid (TFA)), Mobile Phase B: methanol (MeOH); Flow rate: 25 mL/min; Gradient: 60% B to 60% B in 20 min, 60% B; Wave Length: 254 nm; RT(min): 18) to provide purified Compound 23A (10.0 mg). LCMS (ES, m/z): RT=1.63 min, m/z=383.3[M+H]⁺. ¹H NMR (400 MHz, DMSO-d₆) δ 9.43 (s, 1H), 7.10-6.94 (m, 1H), 6.58-6.47 (m, 2H), 6.33 (d, J=8.1 Hz, 1H), 3.81-3.65 (m, 3H), 2.70-2.58 (m, 1H), 2.46-2.37 (m, 1H), 1.87-1.76 (m, 1H), 1.70 (d, J=12.5 Hz, 2H), 1.31 (d, J=2.3 Hz, 2H), 1.12-0.99 (m, 1H), 0.96-0.89 (m, 2H), 0.88-0.78 (m, 5H), 0.63-0.54 (m, 2H).

Compound 23B—OMe and Compound 23B may be synthesized from (3S,5S)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 3 of Example 23.

Compound 23C—OMe and Compound 23C may be synthesized from (3R,5R)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 3 of Example 23.

Compound 23D-OMe and Compound 23D may be synthesized from (3S,5R)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 3 of Example 23.

### Example 24. Synthesis of 1-(2-hydroxy-6-(trifluoromethyl)pyridin-3-yl)-N-((3R,5S)-5-methyl-1-(1H-tetrazol-5-yl)piperidin-3-yl)cyclopropane-1-carboxamide (Compound 24A), 1-(2-methoxy-6-(trifluoromethyl)pyridin-3-yl)-N-((3R,5S)-5-methyl-1-(1H-tetrazol-5-yl)piperidin-3-yl)cyclopropane-1-carboxamide (Compound 24A-OMe), and Compounds 24B, 24C, 24D, 24B—OMe, 24C—OMe, and 24D—OMe

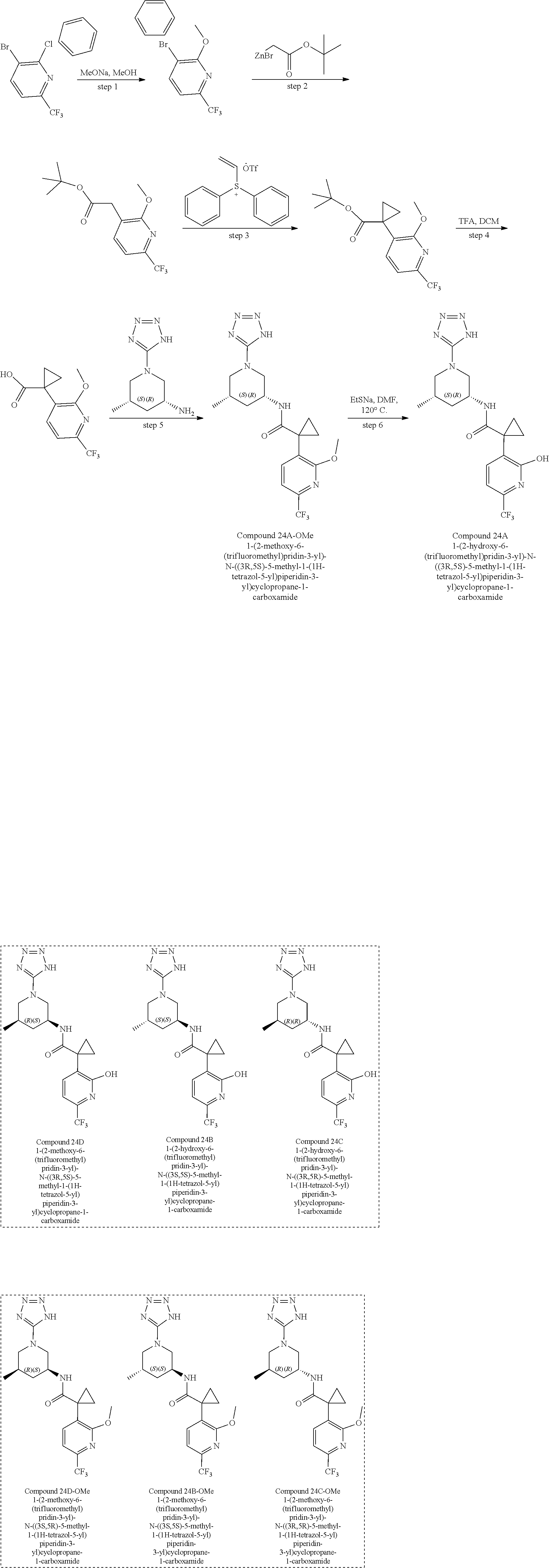

### Example 24 Follows Protocol A

Step 1: Into a 40 mL vial was added 3-bromo-2-chloro-6-(trifluoromethyl)pyridine (1 g, 3.84 mmol, 1 equiv) and sodium methoxide (MeONa) (10 mL). The resulting mixture was stirred for 1 h at 60° C. under air atmosphere. The reaction was monitored by LCMS. The resulting mixture was extracted with ethyl acetate (EtOAc) (3×10 mL). The combined organic layers were washed with water (3×3 mL), dried over anhydrous Na₂SO₄. After filtration, the filtrate was concentrated under reduced pressure. The residue was purified by silica gel column chromatography, eluting with petroleum ether:ethyl acetate (10:1) to provide 3-bromo-2-methoxy-6-(trifluoromethyl)pyridine (780 mg, 79% yield). LCMS (ES, m/z): RT=1.24 min, m/z=256.0[M+H]⁺.

Step 2: Into an 8 mL vial was added 3-bromo-2-methoxy-6-(trifluoromethyl)pyridine (600 mg, 2.34 mmol, 1 equiv), tetrahydrofuran (8 mL), tert-butyl 2-(bromozincio)acetate (1831.06 mg, 7.03 mmol, 3 equiv) and bis(tributylphosphine) palladium (590.07 mg, 0.70 mmol, 0.3 equiv). The resulting mixture was stirred for 2 h at 60° C. under nitrogen atmosphere. The reaction was monitored by LCMS. The resulting mixture was concentrated under reduced pressure. The residue was purified by silica gel column chromatography, eluting with dichloromethane (DCM)/petroleum ether (1:4), to provide tert-butyl 2-[2-methoxy-6-(trifluoromethyl)pyridin-3-yl]acetate (780 mg, >100% yield). LCMS (ES, m/z): RT=1.07 min, m/z=292.0[M+H]⁺.

Step 3: Into an 8 mL vial was added isopropyl 2-[2-methoxy-6-(trifluoromethyl)pyridin-3-yl]acetate (200 mg, 0.72 mmol, 1 equiv), ethenyldiphenylsulfanium (230.83 mg, 1.08 mmol, 1.5 equiv), 1,8-diazabicyclo[5.4.0]undec-7-ene (DBU) (329.47 mg, 2.16 mmol, 3 equiv) and dimethylsulfoxide (DMSO) (3 mL). The resulting mixture was stirred for 1h at 25° C. under air atmosphere. The reaction was monitored by LCMS. The resulting mixture was concentrated under reduced pressure. The residue was purified by Prep-TLC (petroleum ether:ethyl acetate 10:1) to provide tert-butyl 1-[2-methoxy-6-(trifluoromethyl)pyridin-3-yl]cyclopropane-1-carboxylate (170 mg, 74% yield). LCMS (ES, m/z): RT=1.04 min, m/z=318.0[M+H]⁺.

Step 4: Into an 8 mL vial was added tert-butyl 1-[2-methoxy-6-(trifluoromethyl)pyridin-3-yl]cyclopropane-1-carboxylate (150 mg, 0.47 mmol, 1 equiv) and trifluoroacetaldehyde (2 mL). The resulting mixture was stirred for 1 h at 25° C. under air atmosphere. The reaction was monitored by LCMS.

The resulting mixture was extracted with ethyl acetate (EtOAc) (3×5 mL). The combined organic layers were washed with water (3×2 mL), dried over anhydrous Na₂SO₄. After filtration, the filtrate was concentrated under reduced pressure to provide 1-[2-methoxy-6-(trifluoromethyl)pyridin-3-yl]cyclopropane-1-carboxylic acid (120 mg, 97% yield). LCMS (ES, m/z): RT=0.83 min, m/z=262.0[M+H]⁺.

Step 5: Into an 8 mL vial was added 1-[2-methoxy-6-(trifluoromethyl)pyridin-3-yl]cyclopropane-1-carboxylic acid (“carboxyl (ii) reagent”) (78.83 mg, 0.30 mmol, 1.1 equiv), (3R,5S)-5-methyl-1-(1H-1,2,3,4-tetrazol-5-yl)piperidin-3-amine (“amine (i) reagent”) (50 mg, 0.27 mmol, 1 equiv), N,N,N,N′-tetramethylchloroformanidinium hexafluorophosphate (TCFH) (92.38 mg, 0.32 mmol, 1.2 equiv), N-methyl imidazole (NMI) (67.58 mg, 0.82 mmol, 3 equiv) and acetonitrile (MeCN) (3 mL). The resulting mixture was stirred for 1 h at 25° C. under air atmosphere. The reaction was monitored by LCMS. The resulting mixture was extracted with ethyl acetate (EtOAc) (3×5 mL). The combined organic layers were washed with water (3×5 mL), dried over anhydrous Na₂SO₄. After filtration, the filtrate was concentrated under reduced pressure. The residue was purified by Prep-TLC (dichloromethane (DCM)/methanol (MeOH) 5:1) to provide 1-(2-methoxy-6-(trifluoromethyl)pyridin-3-yl)-N-((3R,5S)-5-methyl-1-(1H-tetrazol-5-yl)piperidin-3-yl)cyclopropane-1-carboxamide (Compound 24A-OMe) (90 mg, 77% yield). LCMS (ES, m/z): RT=1.05 min, m/z=426.0[M+H]⁺.

Step 6: Into an 8 mL vial was added Compound 24A-OMe (60 mg, 0.14 mmol, 1 equiv), (ethylsulfanyl)sodium (237.25 mg, 2.82 mmol, 20 equiv) and dimethylformamide (DMF) (2 mL). The resulting mixture was stirred for 1 h at 120° C. under air atmosphere. The reaction was monitored by LCMS. The resulting mixture was extracted with ethyl acetate (EtOAc) (3×5 mL). The combined organic layers were washed with water (3×5 mL), dried over anhydrous Na₂SO₄. After filtration, the filtrate was concentrated under reduced pressure. The crude product (40 mg) was purified by PREP HPLC (XBridge Prep Phenyl OBD Column, 19*250 mm, 5 μm; Mobile Phase A: water (10 mmol/LNH₄HCO₃), Mobile Phase B: methanol (MeOH); Flow rate: 20 mL/min; Gradient: 15% B to 35% B in 10 min, 35% B; Wave Length: 254 nm; RT(min): 9) to provide 1-(2-hydroxy-6-(trifluoromethyl)pyridin-3-yl)-N-((3R,5S)-5-methyl-1-(1H-tetrazol-5-yl)piperidin-3-yl)cyclopropane-1-carboxamide (Compound 24A) (6.2 mg). LCMS (ES, m/z): RT=0.66 min, m/z=412.0[M+H]⁺. ¹H NMR (400 MHz, DMSO-d₆) δ 7.75 (d, J=7.5 Hz, 1H), 7.24 (s, 1H), 6.96 (d, J=8.0 Hz, 1H), 3.78-3.71 (m, 4H), 2.29 (t, J=11.9 Hz, 1H), 1.70 (d, J=12.7 Hz, 2H), 1.39 (t, J=3.9 Hz, 2H), 1.08 (q, J=12.3 Hz, 1H), 0.980.87 (m, 5H).

Compound 24B—OMe and Compound 24B may be synthesized from (3S,5S)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 5 of Example 24.

Compound 24C—OMe and Compound 24C may be synthesized from (3R,5R)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 5 of Example 24.

Compound 24D-OMe and Compound 24D may be synthesized from (3S,5R)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 5 of Example 24.

### Example 25. Synthesis of 1-(3-hydroxy-5-(trifluoromethyl)pyridin-2-yl)-N-((3R,5S)-5-methyl-1-(1H-tetrazol-5-yl)piperidin-3-yl)cyclopropane-1-carboxamide (Compound 25A) and 1-(3-methoxy-5-(trifluoromethyl)pyridin-2-yl)-N-((3R,5S)-5-methyl-1-(1H-tetrazol-5-yl)piperidin-3-yl)cyclopropane-1-carboxamide (Compound 25A-OMe)

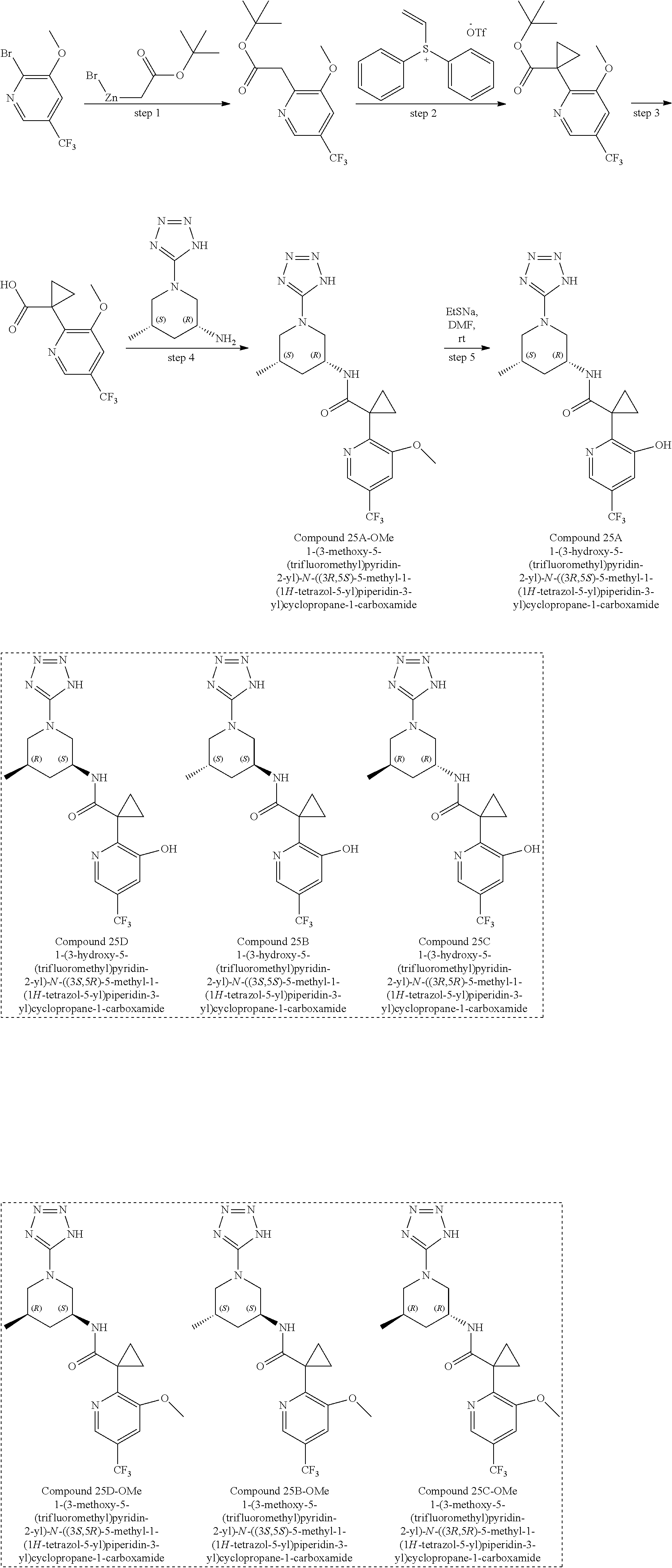

### Example 25 Follows Protocol A

Step 1: Into a 20 mL vial was added 2-bromo-3-methoxy-5-(trifluoromethyl)pyridine (500 mg, 1.953 mmol, 1 equiv), tetrahydrofuran (THF) (6 mL), tert-butyl 2-(bromozincio)acetate (1017.26 mg, 3.906 mmol, 2 equiv) and bis(tri-tert-butylphosphine)palladium(0) (Pd(t-Bu₃P)₂) (299.42 mg, 0.586 mmol, 0.3 equiv) at room temperature under nitrogen atmosphere. The resulting mixture was stirred for 4 h at 60° C. The resulting mixture was concentrated under vacuum. The residue was purified by Prep-TLC (petroleum ether:ethyl acetate 4:1) to provide tert-butyl 2-[3-methoxy-5-(trifluoromethyl)pyridin-2-yl]acetate (270 mg, 47% yield). LCMS (ESI): RT=1.00 min, m/z=292.0 [M⁺H]⁺.

Step 2: Into an 8 mL vial was added tert-butyl 2-[3-methoxy-5-(trifluoromethyl)pyridin-2-yl]acetate (500 mg, 1.72 mmol, 1 equiv), dimethylsulfoxide (DMSO) (7 mL), diphenylethenyl-lambda4-sulfanyl trifluoromethanesulfonate (933.10 mg, 2.58 mmol, 1.5 equiv) and 1,8-diazabicyclo[5.4.0]undec-7-ene (DBU) (522.68 mg, 3.43 mmol, 2 equiv) at room temperature. The resulting mixture was stirred for 2 h at room temperature. The resulting mixture was extracted with ethyl acetate (EtOAc) (3×10 mL). The combined organic layers were washed with brine (1×10 mL), dried over anhydrous Na₂SO₄. After filtration, the filtrate was concentrated under reduced pressure. The residue was purified by Prep-TLC (petroleum ether:ethyl acetate 1:0) to provide tert-butyl 1-[3-methoxy-5-(trifluoromethyl)pyridin-2-yl]cyclopropane-1-carboxylate (160 mg, 29% yield). LCMS (ESI): RT=1.11 min, m/z=318.0 [M⁺H]⁺.

Step 3: Into an 8 mL vial was added tert-butyl 1-[3-methoxy-5-(trifluoromethyl)pyridin-2-yl]cyclopropane-1-carboxylate (160 mg, 0.504 mmol, 1 equiv), dichloromethane (DCM) (1 mL) and trifluoroacetic acid (TFA) (1 mL) at room temperature. The resulting mixture was stirred for 2 h at room temperature. The mixture was basified to pH 8 with saturated NaHCO₃ (aq.). The resulting mixture was extracted with ethyl acetate (EtOAc) (3×10 mL). The combined organic layers were washed with brine (1×10 mL), and dried over anhydrous Na₂SO₄. After filtration, the filtrate was concentrated under reduced pressure. This resulted in 1-[3-methoxy-5-(trifluoromethyl)pyridin-2-yl]cyclopropane-1-carboxylic acid (120 mg, 91% yield). LCMS (ESI): RT=0.73 min, m/z=262.0 [M⁺H]⁺.

Step 4: Into an 8 mL vial was added 1-[3-methoxy-5-(trifluoromethyl)pyridin-2-yl]cyclopropane-1-carboxylic acid (“carboxyl (ii) reagent”) (143.3 g, 0.540 mmol, 1 equiv), dimethyl formamide (DMF) (0.5 mL), hydroxybenzotriazole (HOBT) (148.30 mg, 1.080 mmol, 2 equiv), 1-ethyl-3-(3-dimethylaminopropyl)carbodiimide (EDCI) (210.39 mg, 1.080 mmol, 2 equiv), diisopropylethyl amine (DIEA) (212.77 mg, 1.620 mmol, 3 equiv), and (3R,5S)-5-methyl-1-(1H-1,2,3,4-tetrazol-5-yl)piperidin-3-amine (“amine (i) reagent”) (100 mg, 0.54 mmol, 1 equiv) at room temperature. The resulting mixture was stirred for 2 h at room temperature, then extracted with ethyl acetate (EtOAc) (3×10 mL). The combined organic layers were washed with brine (1×10 mL), dried over anhydrous Na₂SO₄. After filtration, the filtrate was concentrated under reduced pressure. The residue was purified by silica gel column chromatography, eluting with petroleum ether:ethyl acetate (3:7), to provide 1-(3-methoxy-5-(trifluoromethyl)pyridin-2-yl)-N-((3R,5S)-5-methyl-1-(1H-tetrazol-5-yl)piperidin-3-yl)cyclopropane-1-carboxamide (Compound 25A-OMe) (60 mg, 26% yield). LCMS (ESI): RT=0.77 min, m/z=426.0 [M⁺H]⁺.

Step 5: Into an 8 mL vial was added Compound 25A-OMe (50 mg, 0.12 mmol, 1 equiv), dimethyl formamide (DMF) (0.5 mL) and (ethylsulfanyl)sodium (98.86 mg, 1.18 mmol, 10 equiv) at room temperature. The resulting mixture was stirred for 2 h at 120° C. The residue was purified by reverse flash chromatography (C18 silica gel; mobile phase, acetonitrile (MeCN) in water, 10% to 40% gradient in 10 min; detector, UV 254 nm) to provide crude product (40 mg), which was further purified by Chiral-PREP HPLC (YMC-Actus Triart C18 ExRS, 30*150 mm, 5 μm; mobile phase, water (10 mmol/L NH₄HCO₃) and acetonitrile (MeCN) (16% acetonitrile (MeCN) up to 26% in 10 min); Detector, UV254 nm) to provide 1-(3-hydroxy-5-(trifluoromethyl)pyridin-2-yl)-N-((3R,5S)-5-methyl-1-(1H-tetrazol-5-yl)piperidin-3-yl)cyclopropane-1-carboxamide (Compound 25A) (23.3 mg). LCMS (ESI): RT=1.26 min, m/z=413.0 [M+H]⁺. ¹H NMR (400 MHz, Methanol-d₄) δ 8.28 (s, 1H), 7.41 (s, 1H), 4.03-3.72 (m, 3H), 2.60 (t, J=11.5 Hz, 1H), 2.40 (t, J=11.9 Hz, 1H), 1.92 (d, J=12.5 Hz, 1H), 1.82 (s, 1H), 1.55 (d, J=21.6 Hz, 2H), 1.31 (s, 1H), 1.24 (s, 1H), 1.09 (q, J=12.1 Hz, 1H), 0.97 (d, J=6.6 Hz, 3H).

Compound 25B—OMe and Compound 25B may be synthesized from (3S,5S)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 4 of Example 25.

Compound 25C—OMe and Compound 25C may be synthesized from (3R,5R)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 4 of Example 25.

Compound 25D-OMe and Compound 25D may be synthesized from (3S,5R)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 4 of Example 25.

### Example 26. Synthesis of 1-(4-hydroxy-6-(trifluoromethyl)pyridin-3-yl)-N-((3R,5S)-5-methyl-1-(1H-tetrazol-5-yl)piperidin-3-yl)cyclopropane-1-carboxamide (Compound 26A), 1-(4-methoxy-6-(trifluoromethyl)pyridin-3-yl)-N-((3R,5S)-5-methyl-1-(1H-tetrazol-5-yl)piperidin-3-yl)cyclopropane-1-carboxamide (Compound 26A-OMe), and Compounds 26B, 26C, 26D, Compounds 26B—OMe, 26C—OMe, and 26D-OMe

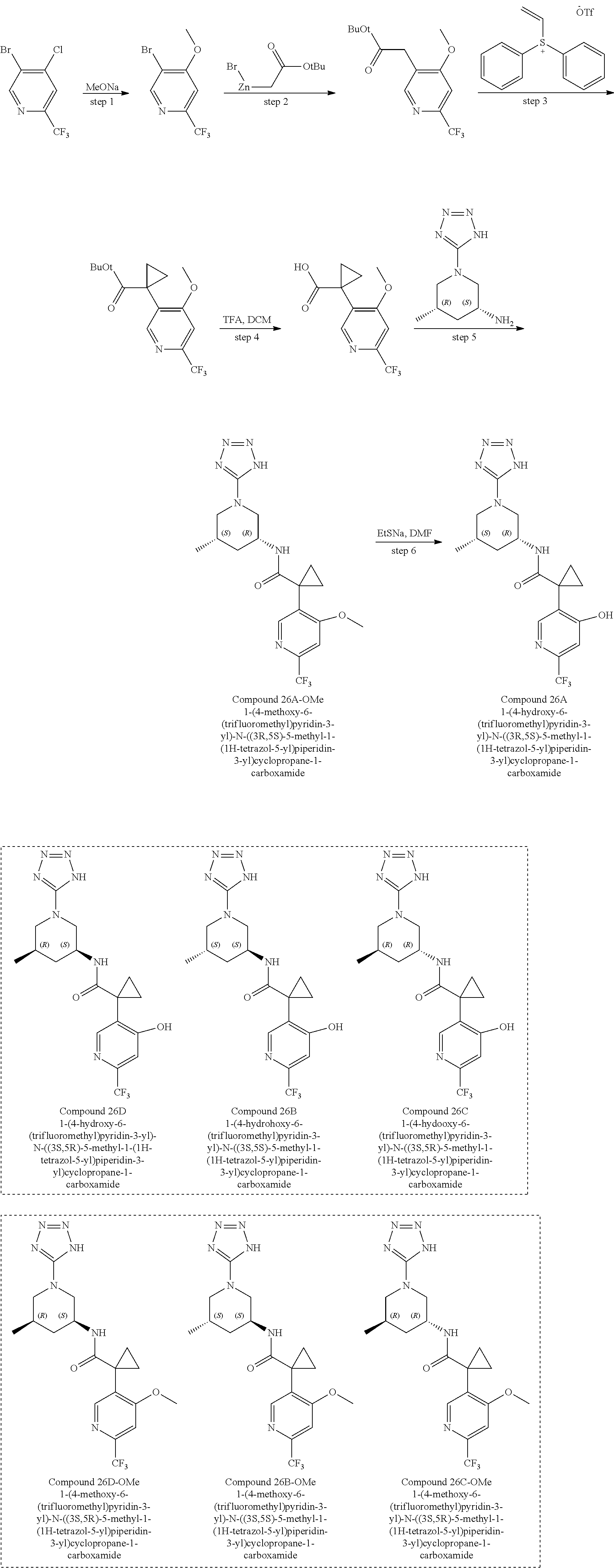

### Example 26 Follows Protocol A

Step 1: Into an 8 mL vial was added 5-bromo-4-chloro-2-(trifluoromethyl)pyridine (500 mg, 1.92 mmol, 1 equiv) and sodium methoxide (MeONa) (5 mL). The resulting mixture was stirred for 1 h at 60° C. The reaction was monitored by LCMS. The resulting mixture was diluted with water (10 mL). The resulting mixture was extracted with dichloromethane (DCM) (2×10 mL). The combined organic layers were washed with water (10 mL), dried over anhydrous Na₂SO₄. After filtration, the filtrate was concentrated under reduced pressure. This resulted in 5-bromo-4-methoxy-2-(trifluoromethyl)pyridine (420 mg, 68% yield). LCMS (ES, m/z): RT=0.86 min, m/z=255.9 [M+1].

Step 2: Into an 8 mL vial was added 5-bromo-4-methoxy-2-(trifluoromethyl)pyridine (250 mg, 0.97 mmol, 1 equiv), tert-butyl 2-(bromozincio)acetate (635.79 mg, 2.44 mmol, 2.5 equiv), bis(tri-tert-butylphosphine)palladium(0) (Pd(P(t-Bu)₃)₂) (149.71 mg, 0.29 mmol, 0.3 equiv), and tetrahydrofuran (THF) (3 mL). The resulting mixture was stirred for 2 h at 60° C. under nitrogen atmosphere. The reaction was monitored by LCMS. The resulting mixture was filtered, the filter cake was washed with ethyl acetate (EtOAc) (3×5 mL). The filtrate was concentrated under reduced pressure. The residue was purified by Prep-TLC (petroleum ether:ethyl acetate 3:1) to provide tert-butyl 2-[4-methoxy-6-(trifluoromethyl)pyridin-3-yl]acetate (300 mg, 84% yield). LCMS (ES, m/z): RT=0.52 min, m/z=292.1 [M+1].

Step 3: Into an 8 mL vial was added tert-butyl 2-[4-methoxy-6-(trifluoromethyl)pyridin-3-yl]acetate (300 mg, 1.03 mmol, 1 equiv), ethenyldiphenylsulfanium triflate (559.86 mg, 1.54 mmol, 1.5 equiv), 1,8-diazabicyclo[5.4.0]undec-7-ene (DB1U) (313.31 mg, 2.06 mmol, 2 equiv), and dimethylsulfoxide (DMSO) (3 mL). The resulting mixture was stirred for 3 h at 25° C. The reaction was monitored by LCMS. The reaction was quenched with water. The resulting mixture was extracted with ethyl acetate (EtOAc) (3×5 mL). The combined organic layers were washed with water (5 mL), dried over anhydrous Na₂SO₄. After filtration, the filtrate was concentrated under reduced pressure. The residue was purified by Prep-TLC (petroleum ether:ethyl acetate 5:1) to provide tert-butyl 1-[4-methoxy-6-(trifluoromethyl)pyridin-3-yl]cyclopropane-1-carboxylate (300 mg, 73% yield). LCMS (ES, m/z): RT=1.00 min, m/z=318.1 [M+1].

Step 4: Into an 8 mL vial was added tert-butyl 1-[4-methoxy-6-(trifluoromethyl)pyridin-3-yl]cyclopropane-1-carboxylate (250 mg, 0.78 mmol, 1 equiv), trifluoroacetic acid (TFA) (2 mL), and dichloromethane (DCM) (2 mL). The resulting mixture was stirred for 2 h at 25° C. The reaction was monitored by LCMS. The resulting mixture was concentrated under reduced pressure. The resulting mixture was extracted with ethyl acetate (EtOAc) (3×5 mL), dried over anhydrous Na₂SO₄. After filtration, the filtrate was concentrated under reduced pressure. This resulted in 1-[4-methoxy-6-(trifluoromethyl)pyridin-3-yl]cyclopropane-1-carboxylic acid (200 mg, 78% yield). LCMS (ES, m/z): RT=0.68 min, m/z=262.1 [M+1].

Step 5: Into an 8 mL vial was added 1-[4-methoxy-6-(trifluoromethyl)pyridin-3-yl]cyclopropane-1-carboxylic acid (“carboxyl (ii) reagent”) (100 mg, 0.38 mmol, 1 equiv) and (3R,5S)-5-methyl-1-(1H-1,2,3,4-tetrazol-5-yl)piperidin-3-amine (“amine (i) reagent”) (69.77 mg, 0.38 mmol, 1 equiv), N,N,N′,N′-tetramethylchloroformamidinium hexatluorophosphate (TCFH) (128.90 mg, 0.46 mmol, 1.2 equiv), N-methyl imidazole (NMI) (94.30 mg, 1.14 mmol, 3 equiv), and acetonitrile (MeCN) (1 mL). The resulting mixture was stirred for 2 h at 25° C. The reaction was monitored by LCMS. The resulting mixture was extracted with ethyl acetate (EtOAc) (3×5 mL), dried over anhydrous Na₂SO₄. After filtration, the filtrate was concentrated under reduced pressure. The residue was purified by silica gel column chromatography, eluting with dichloromethane (DCM)/methanol (MeOH) (10:1) to provide 1-(4-methoxy-6-(trifluoromethyl)pyridin-3-yl)-N-((3R,5S)-5-methyl-1-(1H-tetrazol-5-yl)piperidin-3-yl)cyclopropane-1-carboxamide (Compound 26A-OMe) (90 mg, 39% yield). LCMS (ES, m/z): RT=0.73 min, m/z=426.2 [M+1].

Step 6: Into an 8 mL vial was added Compound 26A-OMe (80 mg, 0.18 mmol, 1 equiv), sodium ethylsulfanide (237.25 mg, 2.82 mmol, 15 equiv), and dimethyl formamide (DMF) (1 mL). The resulting mixture was stirred for 1 h at 120° C. The reaction was monitored by LCMS. The resulting mixture was filtered, the filter cake was washed with ethyl acetate (EtOAc) (3×5 mL), and the filtrate was concentrated under reduced pressure. The residue was purified by reverse flash chromatography (C18 silica gel; mobile phase, acetonitrile (MeCN) in water, 10% to 80% gradient in 25 min; detector, UV 254 nm) to provide 1-(4-hydroxy-6-(trifluoromethyl)pyridin-3-yl)-N-((3R,5S)-5-methyl-1-(1H-tetrazol-5-yl)piperidin-3-yl)cyclopropane-1-carboxamide (Compound 26A) (45 mg, 80% purity). The crude product (45 mg) was purified by PREP HPLC (XBridge Shield RP18 OBD Column, 30*150 mm, 5 μm; mobile phase, water (10 mmol/L NH₄HCO₃) and acetonitrile (MeCN) (3% acetonitrile (MeCN) up to 15% in 10 min); Detector, UV 254 nm) to provide purified Compound 26A (8.8 mg). LCMS (ES, m/z): RT=0.74 min, m/z=412.2 [M+1]⁺. ¹H NMR (400 MHz, DMSO-d6) δ 8.12 (s, 1H), 8.04 (s, 1H), 6.79 (s, 1H), 3.86-3.82 (m, 3H), 3.75 (d, J=12.4 Hz, 1H), 2.38-2.31 (m, 1H), 1.82-1.65 (m, 2H), 1.32-1.19 (m, 2H), 1.04-0.92 (m, 1H), 0.86 (d, J=6.4 Hz, 3H), 0.80 (d, J=2.4 Hz, 2H).

Compound 26B—OMe and Compound 26B may be synthesized from (3S,5S)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 5 of Example 26.

Compound 26C—OMe and Compound 26C may be synthesized from (3R,5R)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 5 of Example 26.

Compound 26D-OMe and Compound 26D may be synthesized from (3S,5R)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 5 of Example 26.

### Example 27. Synthesis of 1-(2-hydroxy-4-isopropylphenyl)-N-(5-methyl-1-(1H-tetrazol-5-yl)azepan-3-yl)cyclopropane-1-carboxamide (Compound 27, rac-27), 1-(4-isopropyl-2-methoxyphenyl)-N-(5-methyl-1-(1H-tetrazol-5-yl)azepan-3-yl)cyclopropane-1-carboxamide (Compound 27-OMe, rac-27-OMe), Compounds 27A*, 27B*, 27C*, and 27D*, and Compounds 27A-OMe*, 27B—OMe*, 27C—OMe*, and 27D-OMe*

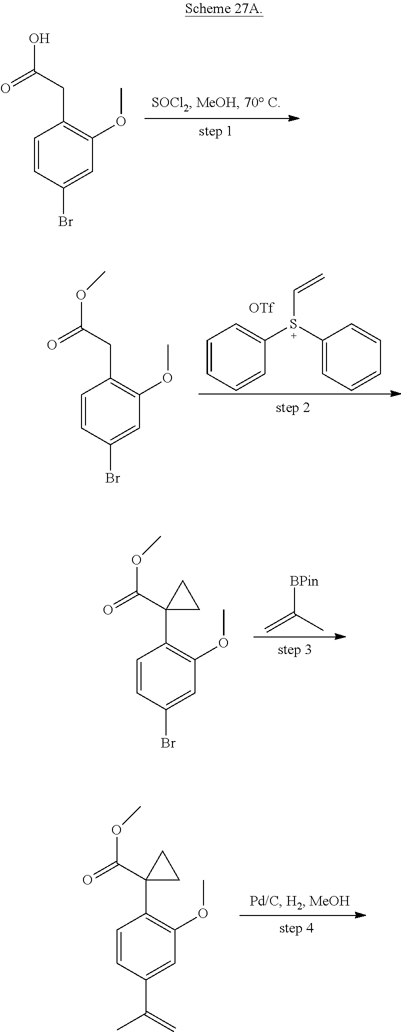

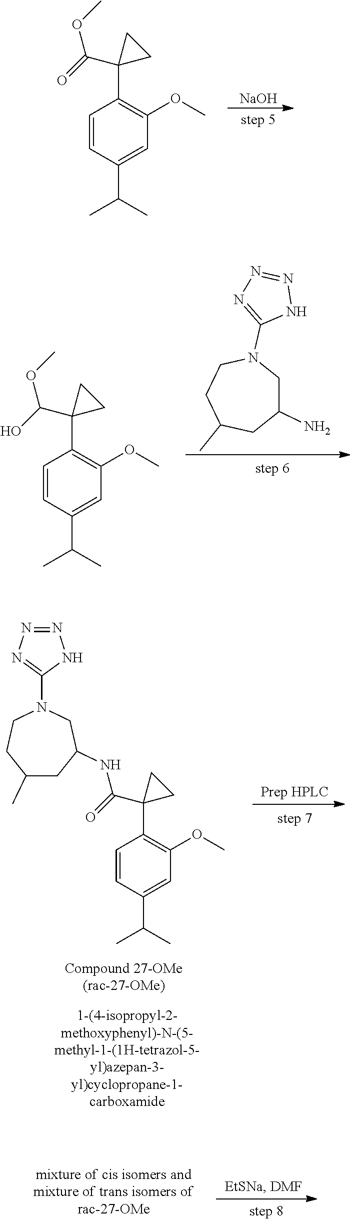

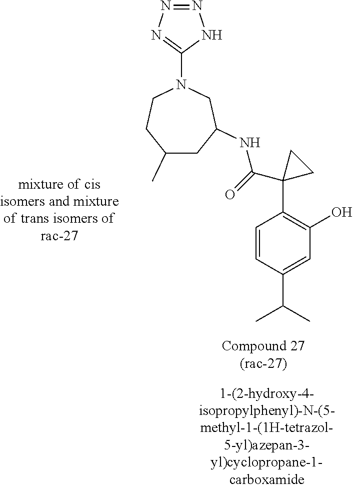 [SMILES: CC1CCN(c2nnn[nH]2)CC(NC(=O)C2(c3ccc(C(C)C)cc3O)CC2)C1]

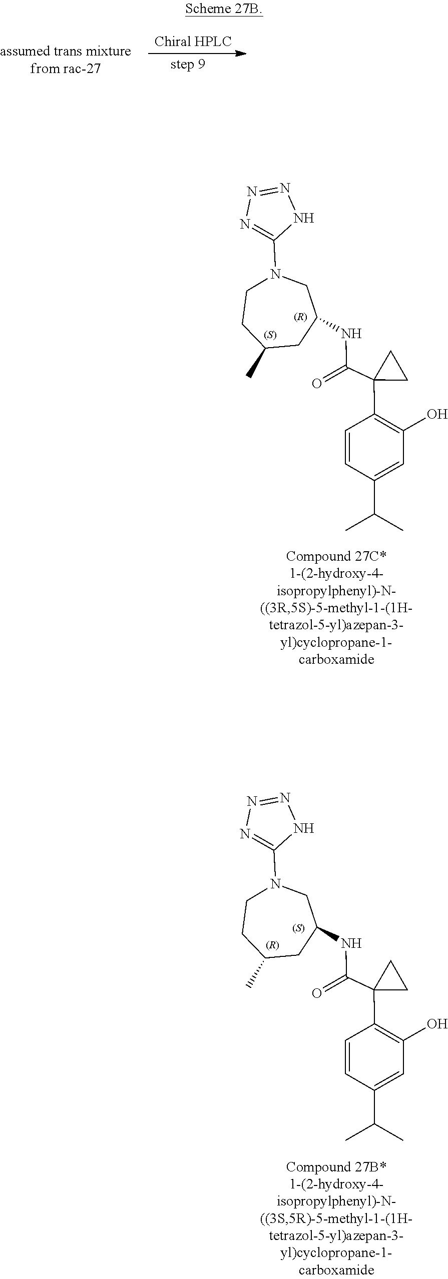

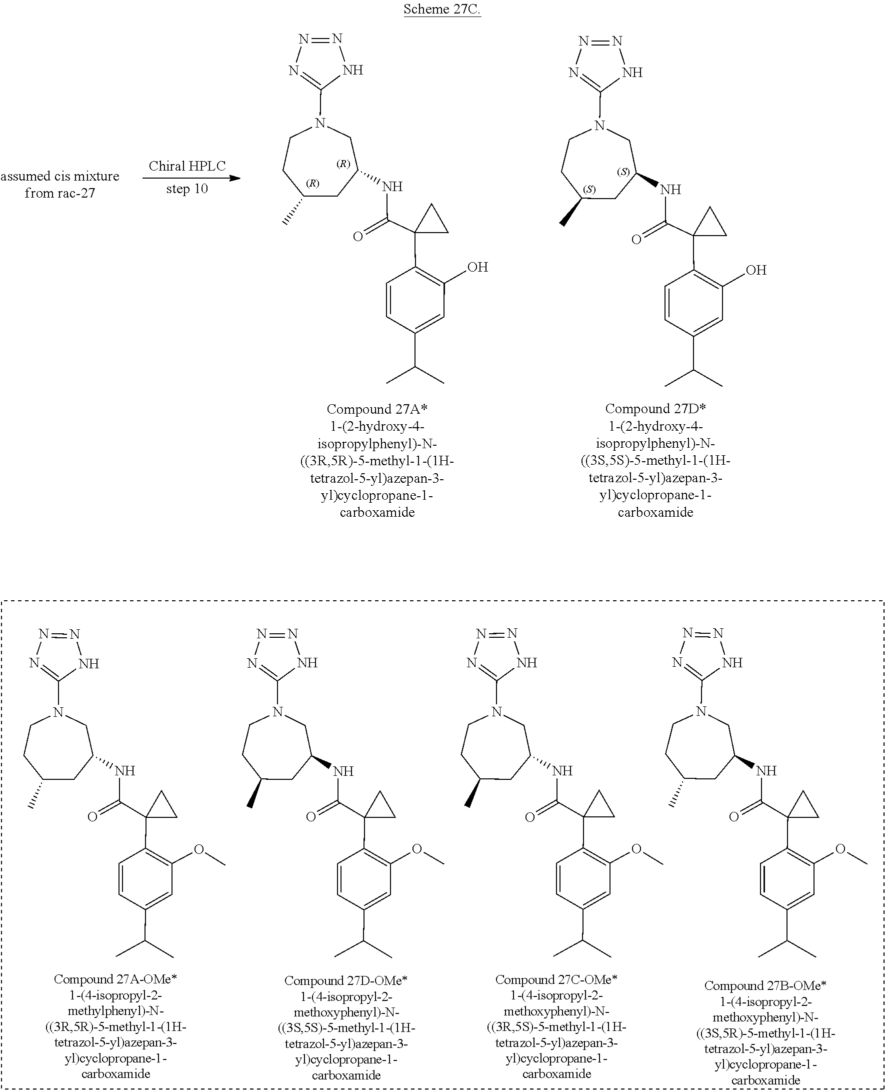

### Example 27 Follows Protocol A

Step 1: Into a 100 mL round-bottom flask was added 4-bromo-2-methoxyphenyl)acetic acid (6 g, 24.48 mmol, 1 equiv), SOCl₂ (5 mL), and methanol (MeOH) (30 mL) at room temperature. The resulting mixture was stirred for 2 h at 80° C. The reaction was monitored by LCMS. The resulting mixture was concentrated under reduced pressure. The residue was purified by silica gel column chromatography, eluting with petroleum ether/ethyl acetate (1:1) to provide methyl 2-(4-bromo-2-methoxyphenyl)acetate (5.16 g, 81% yield). LCMS (ES, m/z): RT=1.078 min, m/z=258 [M+1]⁺.

Step 2: Into a 40 mL vial was added methyl 2-(4-bromo-2-methoxyphenyl)acetate (2 g, 7.72 mmol, 1 equiv), ethenyldiphenylsulfanium (1.98 g, 9.26 mmol, 1.2 equiv), 1,8-diazabicyclo[5.4.0]undec-7-ene (DBU) (3.53 g, 23.15 mmol, 3 equiv), and dimethylsulfoxide (DMSO) (16 mL) at room temperature. The resulting mixture was stirred for 2 h at room temperature. The reaction was monitored by LCMS. The residue was purified by reverse flash chromatography (C18 silica gel; mobile phase, acetonitrile (MeCN) in water, 0% to 100% gradient in 30 min; detector, UV 254 nm) to provide methyl 1-(4-bromo-2-methoxyphenyl)cyclopropane-1-carboxylate (1 g, 45% yield). LCMS:(ES, m/z): RT=1.119 min, m/z=285[M+1]⁺.

Step 3: Into an 8 mL vial was added methyl 1-(4-bromo-2-methoxyphenyl)cyclopropane-1-carboxylate (580 mg, 2.03 mmol, 1 equiv), Na₂CO₃ (646.77 mg, 6.10 mmol, 3 equiv), [1,1′-bis(diphenylphosphino)ferrocene]palladiurn(II) dichloride (Pd(dppf)Cl₂) (148.84 mg, 0.20 mmol, 0.1 equiv), dioxane (10 mL), and water (2 mL) at room temperature. The resulting mixture was stirred for 1h at 80° C. under nitrogen atmosphere. The reaction was monitored by LCMS. The residue was purified by reverse flash chromatography (C18 silica gel; mobile phase, acetonitrile (MeCN) in water, 0% to 100% gradient in 30 min; detector, UV 254 nm) to provide methyl 1-[2-methoxy-4-(prop-1-en-2-yl)phenyl]cyclopropane-1-carboxylate (440 mg, 88% yield). LCMS:(ES, m/z): RT=0.930 min, m/z=247[M+1]⁺.

Step 4: To a solution of methyl 1-[2-methoxy-4-(prop-1-en-2-yl)phenyl]cyclopropane-1-carboxylate (400 mg, 1.61 mmol, 1 equiv) in methanol (MeOH) (50 mL) was added Pd/C (10%, 40 mg) under nitrogen atmosphere in a 100 mL round-bottom flask. The mixture was hydrogenated at room temperature for 1 h under hydrogen atmosphere using a hydrogen balloon. The reaction was monitored by LCMS. The reaction was filtered through a Celite pad and concentrated under reduced pressure to provide the crude product, methyl 1-(4-isopropyl-2-methoxyphenyl)cyclopropane-1-carboxylate (500 mg), which was used in the next step directly without further purification. LCMS:(ES, m/z): RT=0.979 min, m/z=249[M+1]⁺.

Step 5: Into a 8 mL microwave was added methyl 1-(4-isopropyl-2-methoxyphenyl) cyclopropane-1-carboxylate (300 mg, 1.2 mmol, 1 equiv), NaOH (241.60 mg, 6.05 mmol, 15 equiv), methanol (MeOH) (3 mL), and water (0.6 mL) at room temperature. The final reaction mixture was stirred for 2 h at 80° C. The reaction was monitored by LCMS. The reaction mixture was adjusted to pH 5 with HCl (1 mL, 1M). The resulting mixture was extracted with dichloromethane (DCM) (2×50 mL), and dried over anhydrous Na₂SO₄. The resulting mixture was concentrated under reduced pressure to provide 1-(4-isopropyl-2-methoxyphenyl)cyclopropane-1-carboxylic acid (200 mg, 74% yield). LCMS:(ES, m/z): RT=0.916 min, m/z=235[M+1]+.

Step 6: Into a 20 mL sealed tube was added 1-(4-isopropyl-2-methoxyphenyl)cyclopropane-1-carboxylic acid (“carboxyl (ii) reagent”) (200 mg, 0.86 mmol, 1 equiv), dimethyl formamide (DMF) (7.5 mL), 5-methyl-1-(1H-1,2,3,4-tetrazol-5-yl)azepan-3-amine (“amine (i) reagent”) (301.56 mg, 1.54 mmol, 1.2 equiv), hydroxybenzotriazole (HOBT) (346.04 mg, 2.56 mmol, 2 equiv), diisopropylethyl amine (DIEA) (496.48 mg, 3.84 mmol, 3 equiv), and 1-ethyl-3-(3-dimethylaninopropyl)carbodiimide (EDCI) (490.92 mg, 2.56 mmol, 2 equiv) at room temperature. The resulting mixture was stirred for 1 h at room temperature. The reaction was monitored by LCMS. The residue was concentrated under reduced pressure to provide 1-(4-isopropyl-2-methoxyphenyl)-N-(5-methyl-1-(1H-tetrazol-5-yl)azepan-3-yl)cyclopropane-1-carboxamide (Compound 27-OMe, rac-27-OMe) as a mixture of cis and trans isomers.

Step 7: The product, rac-27-OMe, was purified by reverse flash chromatography (C18 silica gel; mobile phase, acetonitrile (MeCN) in water (10 mmol/L NH₄HCO₃), 0% to 100% gradient in 30 min; detector, UV 254 nm) to provide a mixture of assumed trans isomers, Compounds 27B—OMe* and 27C—OMe* (70 mg, 32% yield) and assumed cis isomers as first eluting peaks, and Compounds 27A-OMe* and 27D-OMe* (25 mg, 13% yield) as second eluting peaks, cis and trans stereochemistry arbitrarily assigned. LCMS:(ES, m/z): RT=0.827 min (trans), m/z=413[M+1]+. LCMS:(ES, m/z): RT=0.909 min (cis), m/z=413[M+1]+.

Step 8A: Into a 20 mL sealed tube was added the assumed cis rac-27-OMe mixture (70 mg, 0.17 mmol, 1.00 equiv),(ethylsulfanyl)sodium (279 mg, 3.32 mmol, 20 equiv) and DMSO (2.00 mL) at room temperature. The resulting mixture was stirred for 3 h at 120° C. The reaction was monitored by LCMS. The reaction was quenched with water (20 mL) at room temperature. The resulting mixture was extracted with ethyl acetate (EtOAc) (2×20 mL). The combined organic layers were washed with water (1×20 mL), dried over anhydrous Na₂SO₄. After filtration, the filtrate was concentrated under reduced pressure. The residue was purified by reverse flash chromatography (C18 silica gel; mobile phase, acetonitrile in water (0.1% NH₃·H₂O), 0% to 100% gradient in 40 min; detector, UV 254/220 nm) to provide a mixture of Compound 27A* and Compound 27D* (40 mg, 82% purity), which was further purified by PREP HPLC (XBridge Prep Phenyl OBD Column, 19*250 mm, 5 μm; Mobile Phase A: water (0.05% trifluoroacetic acid), Mobile Phase B: acetonitrile; Flow rate: 25 mL/min; Gradient: 40% B to 50% B in 10 min; Wave Length: 220/254 nm; RT(min): 9) to provide a purified mixture of Compound 27A* and Compound 27D* (25 mg, 37% yield). LCMS (ES, m/z): RT=0.821 min, m/z=399[M+1]+. Stereochemistry arbitrarily assigned.

Step 8B: The assumed trans rac-27-OMe mixture was deprotected following the procedure of Step 8A, to provide a mixture of Compound 27B* and Compound 27C*. LCMS (ES, m/z): RT=1.432 min, m/z=399[M+1]+.

Step 9: The assumed trans mixture of rac-27 (40 mg, 97% purity) was separated by chiral HPLC (CHIRALART Cellulose-SC, 2*25 cm, 5 μm; Mobile Phase A: Hexanes (0.1% trifluoroacetic acid (TFA), Mobile Phase B: isopropanol:dichloromethane=1:1; Flow rate: 20 mL/min; Gradient: 25% B to 25% B in 19 min; Wave Length: 220/254 nm) to provide Compound 27B* (RT(min): 12.53, 10 mg) as first eluting peak and Compound 27C* (RT(min): 16.47, 11 mg) as second eluting peak. Stereochemistry was arbitrarily assigned.

Compound 27B*: LCMS: (ES, m/z):RT=0.744 min, m/z=513 [M+1]⁺. ¹H NMR (400 MHz, Methanol-d4) δ 6.91 (d, J=7.9 Hz, 1H), 6.62 (d, J=1.7 Hz, 1H), 6.55-6.52 (m, 1H), 4.17-4.13 (m, 1H), 3.69-3.66 (m, 1H), 3.58-3.52 (m, 1H), 3.46-3.42 (m, 1H), 3.13-3.10 (m, 1H), 2.79-2.75 (m, 1H), 1.77-1.73 (m, 2H), 1.69-1.51 (m, 3H), 1.44-1.30 (m, 2H), 1.22 (d, J=6.9 Hz, 6H), 1.11-1.01 (m, 1H), 0.98 (d, J=6.4 Hz, 3H), 0.90-0.84 (m, 1H).

Compound 27C*: LCMS: (ES, m/z):RT=0.742 min, m/z=513 [M+1]⁺. ¹H NMR (400 MHz, Methanol-d4) δ 6.91 (d, J=7.8 Hz, 1H), 6.62 (d, J=1.7 Hz, 1H), 6.55-6.51 (m, 1H), 4.17-4.12 (m, 1H), 3.69-3.65 (m, 1H), 3.58-3.54 (m, 1H), 3.46-3.41 (m, 1H), 3.13-3.07 (m, 1H), 2.78-2.72 (m, 1H), 1.82-1.70 (m, 2H), 1.70-1.49 (m, 3H), 1.40-1.28 (m, 2H), 1.22 (d, J=6.9 Hz, 6H), 1.05-1.00 (m, 1H), 0.98 (d, J=6.4 Hz, 3H), 0.90-0.85 (m, 1H).

Step 10: The assumed cis mixture of Compound 27A* and Compound 27D* (25 mg, 98% purity) was separated by chiral HPLC (Lux 5 μm Cellulose-4, 2.12*25 cm, 5 μm; Mobile Phase A: Hexanes (0.1% trifluoroacetic acid (TFA)), Mobile Phase B: isopropanol (IPA); Flow rate: 20 mL/min; Gradient: 30% B to 30% B in 20 min; Wave Length: 220/254 nm) to provide Compound 27A* (RT(min): 11.82, 9.1 mg), and Compound 27D* (RT(min): 7.63, 9.7 mg). Stereochemistry was arbitrarily assigned.

Compound 27A*: LCMS: (ES, m/z):RT=1.074 min, m/z=513 [M+1]⁺. ¹H NMR (400 MHz, Methanol-d4) δ 7.14 (d, J=8.3 Hz, 1H), 6.75-6.65 (m, 2H), 3.94-3.85 (m, 1H), 3.63-3.48 (m, 3H), 3.40-3.57 (m, 1H), 2.86-2.74 (m, 1H), 1.91-1.82 (m, 1H), 1.75-1.66 (m, 1H), 1.66-1.58 (m, 1H), 1.58-1.49 (m, 3H), 1.36-29 (m, 1H), 1.25 (d, J=6.9 Hz, 6H), 1.05-1.00 (m, 2H), 0.97 (d, J=6.7 Hz, 3H).

Compound 27D*: LCMS: (ES, m/z):RT=1.120 min, m/z=513 [M+1]⁺. ¹H NMR (400 MHz, Methanol-d4) δ 7.14 (d, J=8.2 Hz, 1H), 6.75-6.68 (m, 2H), 3.94-3.86 (m, 1H), 3.63-3.48 (m, 3H), 3.40-3.29 (m, 1H), 2.85-2.76 (m, 1H), 1.90-1.81 (m, 1H), 1.70-1.63 (m, 1H), 1.62-1.56 (m, 1H), 1.58-1.46 (m, 3H), 1.25 (d, J=6.9 Hz, 7H), 1.08-1.00 (m, 2H), 0.97 (d, J=6.6 Hz, 3H).

### Example 28. Synthesis of 1-(3-fluoro-2-hydroxy-4-(trifluoromethoxy)phenyl)-N-(5-methyl-1-(1H-tetrazol-5-yl)azepan-3-yl)cyclopropane-1-carboxamide (Compound 28, rac-28), 1-(3-fluoro-2-methoxy-4-(trifluoromethoxy)phenyl)-N-(5-methyl-1-(1H-tetrazol-5-yl)azepan-3-yl)cyclopropane-1-carboxamide (Compound 28-OMe, rac-28-OMe), Compounds 28A*, 28B*, 28C*, and 28D*, and Compounds 28A-OMe*, 28B—OMe*, 28C—OMe*, and 28D-OMe*

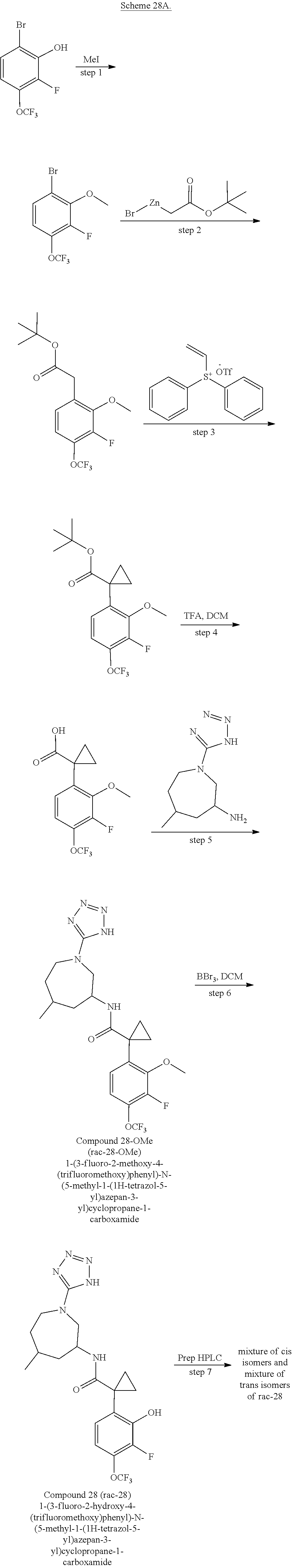

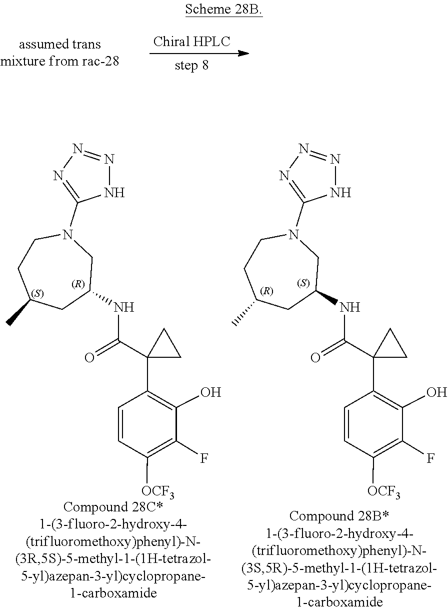

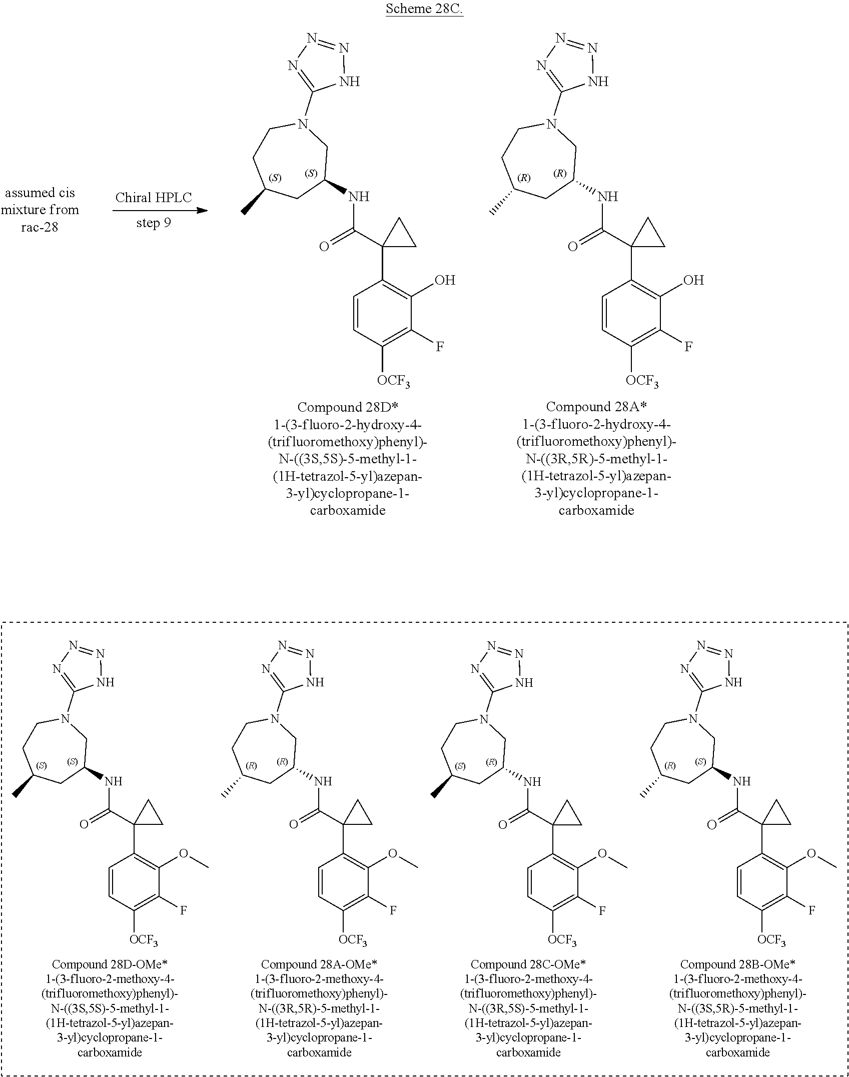

### Example 28 Follows Protocol A

Step 1: Into a 40 mL sealed tube was added 6-bromo-2-fluoro-3-(trifluoromethoxy)phenol (800 mg, 2.909 mmol, 1 equiv), K₂CO₃ (1206.17 mg, 8.727 mmol, 3 equiv) and acetonitrile (MeCN) (5 mL) at room temperature. The resulting mixture was stirred for 1 h at 80° C. under air atmosphere. Desired product could be detected by GCMS. The resulting mixture was extracted with diethyl ether (Et₂O) (3×30 mL), dried over anhydrous Na₂SO₄, and concentrated to provide the crude product, 1-bromo-3-fluoro-2-methoxy-4-(trifluoromethoxy)benzene, which was used in the next step directly without further purification. LCMS (ESI): RT=1.11 min, m/z=290 [M+H]⁺.

Step 2: Into a 40 mL sealed tube was added 1-bromo-3-fluoro-2-methoxy-4-(trifluoromethoxy)benzene (430 mg, 1.488 mmol, 1 equiv), tert-butyl 2-(bromozincio)acetate (1162.42 mg, 4.464 mmol, 3 equiv), bis(tri-tert-butylphosphine)palladium(0) (Pd(t-Bu₃P)₂) (6 mL, 0.446 mmol) and tetrahydrofuran (THF) (21.5 mL) at room temperature. The resulting mixture was stirred for 2 h at 60° C. under nitrogen atmosphere. The resulting mixture was extracted with ethyl acetate (EtOAc) (3×150 mL), and dried over anhydrous Na₂SO₄. After filtration, the filtrate was concentrated under reduced pressure. The residue was purified by silica gel column chromatography, eluting with petroleum ether:ethyl acetate (9:1), to provide tert-butyl 2-[3-fluoro-2-methoxy-4-(trifluoromethoxy)phenyl]acetate (380 mg, 63% yield). LCMS (ESI): RT=1.17 min, m/z=325 [M+H]⁺.

Step 3: Into an 8 mL sealed tube was added tert-butyl 2-[3-fluoro-2-methoxy-4-(trifluoromethoxy)phenyl]acetate (360 mg, 1.110 mmol, 1 equiv), Diphenyl(vinyl)sulfonium trifluoromethanesulfonate (660 mg, 1.65 mmol, 1.5 equiv), 1,8-diazabicyclo[5.4.0]undec-7-ene (DBU) (507.04 mg, 3.330 mmol, 3 equiv) and dimethylsulfoxide (DMSO) (5 mL) at room temperature. The reaction was monitored by LCMS. The reaction was quenched with water(50 mL). The resulting mixture was extracted with EtOAc (3×100 mL), dried over anhydrous Na₂SO₄. After filtration, the filtrate was concentrated under reduced pressure. The residue was purified by reverse flash chromatography (C18 silica gel; mobile phase, acetonitrile (MeCN) in water, 62% to 67% gradient in 4 min; detector, UV 254 nm and 220 nm) to provide tert-butyl 1-[3-fluoro-2-methoxy-4-(trifluoromethoxy)phenyl]cyclopropane-1-carboxylate (300 mg, 77% yield). LCMS (ESI): RT=1.23 min, m/z=351 [M+H]⁺.

Step 4: Into a 20 mL sealed tube was added tert-butyl 1-[3-fluoro-2-methoxy-4-(trifluoromethoxy)phenyl]cyclopropane-1-carboxylate (280 mg, 0.799 mmol, 1 equiv), trifluoroacetic acid (TFA) (1 mL) and dichloromethane (DCM) (5 mL) at room temperature. The resulting mixture was stirred for 1 h at room temperature under air atmosphere. The resulting mixture was concentrated under reduced pressure to provide the crude product, 1-(3-fluoro-2-methoxy-4-(trifluoromethoxy)phenyl) cyclopropane-1-carboxylic acid, which was used in the next step directly without further purification. LCMS (ESI): RT=0.69 min, m/z=293 [M+H]⁺.

Step 5: Into a 20 mL sealed tube was added 1-[3-fluoro-2-methoxy-4-(trifluoromethoxy)phenyl]cyclopropane-1-carboxylic acid (“carboxyl (ii) reagent”) (200 mg, 0.680 mmol, 1 equiv), 5-methyl-1-(1H-1,2,3,4-tetrazol-5-yl)azepan-3-amine (“amine (i) reagent”) (200.13 mg, 1.020 mmol, 1.5 equiv), (1-[bis(dimethylamino)methylene]-1H-12,3-triazolo[4,5-b]pyridinium 3-oxide hexafluorophosphate (HATU) (310.18 mg, 0.816 mmol, 1.2 equiv), triethylamine (TEA) (343.96 mg, 3.4 mmol, 5 equiv) and dimethyl formamide (DMF) (5 mL) at room temperature. The resulting mixture was stirred for 1 h at room temperature under air atmosphere. The residue was purified by reverse flash chromatography (C18 silica gel; mobile phase, acetonitrile (MeCN) in water, 20% to 40% gradient in 20 min; detector, UV 254 nm and 220 nm) to provide 1-(3-fluoro-2-methoxy-4-(trifluoromethoxy)phenyl)-N-(5-methyl-1-(1H-tetrazol-5-yl)azepan-3-yl)cyclopropane-1-carboxamide (Compound 28-OMe, rac-28-OMe) (160 mg, 50% yield) as a mixture of cis and trans isomers. LCMS (ESI): RT=0.94 min, m/z=473 [M+H]⁺.

Step 6: To a stirred mixture of rac-28-OMe (150 mg, 0.317 mmol, 1 equiv) in dichloromethane (DCM) was added BBr₃ (0.95 mL, 0.951 mmol, 3 equiv) in portions at 0° C. The resulting mixture was stirred for 1 h at room temperature under air atmosphere. The reaction was quenched with water at room temperature. The resulting mixture was extracted with ethyl acetate (EtOAc) (3×100 mL), dried over anhydrous Na₂SO₄, filtered, and the filtrate was concentrated under reduced pressure to provide 1-(3-fluoro-2-hydroxy-4-(trifluoromethoxy)phenyl)-N-(5-methyl-1-(1H-tetrazol-5-yl)azepan-3-yl)cyclopropane-1-carboxamide (Compound 28, rac-28) as a mixture of cis and trans isomers.

Step 7: The cis and trans mixture of rac-28 was purified by reverse flash chromatography (C18 silica gel; mobile phase, acetonitrile (MeCN) in water, 30% to 40% gradient in 10 min; detector, UV 254 nm and 220 nm) to provide the assumed trans mixture (35 mg, 28%) as first eluting peak and the assumed cis mixture (25 mg, 19%) as the second eluting peak, stereochemistry arbitrarily assigned.

Step 8: The assumed mixture of trans isomers of rac-28 was separated by chiral HPLC (CHIRALPAK IH, 2*25 cm, 5 gm; Mobile Phase A: Hexanes (0.2% trifluoroacetic acid (TFA)), Mobile Phase B: isopropanol (IPA): dichloromethane (DCM)=1:1; Flow rate: 20 mL/min; Gradient: 25% B to 25% B in 24 min; Wave Length: 220/254 nm) to provide Compound 28C* (RT(min): 17.95, 11.4 mg) and Compound 28B* (RT(min): 8.178, 12.2 mg), stereochemistry arbitrarily assigned.

Compound 28B*: LCMS (ESI): RT=0.83 min, m/z=459 [M+H]⁺; ¹H NMR (400 MHz, Methanol-d4) δ 6.94 (dd, J=8.6, 2.1 Hz, 1H), 6.72 (ddd, J=8.8, 7.2, 1.4 Hz, 1H), 4.19 (s, 1H), 3.74 (dd, J=15.0, 6.1 Hz, 1H), 3.59 (dd, J=15.0, 2.7 Hz, 1H), 3.55-3.46 (m, 1H), 3.34 (s, 7H), 3.22 (ddd, J=14.0, 9.7, 4.9 Hz, 1H), 1.84-1.76 (m, 2H), 1.67 (s, 3H), 1.64 (dd, J=9.7, 5.2 Hz, 1H), 1.59 (td, J=6.6, 3.5 Hz, 1H), 1.53-1.36 (m, 2H), 1.11 (ddd, J=10.6, 6.9, 4.0 Hz, 1H), 1.02-0.91 (m, 4H).

Compound 28C*: LCMS (ESI): RT=1.50 min, m/z=459 [M+H]⁺; ¹H NMR (400 MHz, Methanol-d4) δ 6.94 (dd, J=8.7, 2.1 Hz, 1H), 6.72 (ddd, J=8.6, 7.1, 1.4 Hz, 1H), 4.20 (t, J=5.9 Hz, 1H), 3.74 (dd, J=15.0, 6.0 Hz, 1H), 3.68-3.46 (m, 2H), 3.22 (ddd, J=14.1, 9.8, 4.9 Hz, 1H), 1.85-1.76 (m, 2H), 1.69-1.55 (m, 4H), 1.53-1.37 (m, 1H), 1.11 (ddd, J=9.4, 6.8, 4.0 Hz, 1H), 1.02-0.91 (m, 4H).

Step 9: The assumed mixture of cis isomers from rac-28 was separated by chiral HPLC (LUX 5 μm Cellulose-4, 2.12*25 cm, 5 gm; Mobile Phase A: Hexanes (0.1% trifluoroacetic acid (TFA)), Mobile Phase B: isopropanol (IPA); Flow rate: 20 mL/min; Gradient: 20% B to 20% B in 16 min; Wave Length: 220/254 nm) to provide Compound 28A* (RT(min): 16.51, 9.7 mg) and Compound 28D* (RT(min): 13.18, 9.2 mg), stereochemistry arbitrarily assigned.

Compound 28A*: LCMS (ESI): RT=1.02 min, m/z=459 [M+H]⁺; ¹H NMR (400 MHz, Methanol-d4) δ 7.12 (dd, J=8.7, 2.1 Hz, 1H), 6.88 (dd, J=8.8, 7.0 Hz, 1H), 4.03 (s, 1H), 3.70-3.54 (m, 3H), 3.54-3.43 (m, 1H), 1.84 (d, J=14.5 Hz, 1H), 1.72 (d, J=15.4 Hz, 1H), 1.64-1.55 (m, 3H), 1.58-1.43 (m, 1H), 1.27 (dq, J=24.1, 12.8, 11.6 Hz, 1H), 1.13-1.02 (m, 2H), 0.98 (d, J=6.7 Hz, 3H).

Compound 28D*: LCMS (ESI): RT=1.01 min, m/z=459 [M+H]⁺; ¹H NMR (400 MHz, Methanol-d4) δ 7.12 (dd, J=8.7, 2.1 Hz, 1H), 6.92-6.83 (m, 1H), 4.02 (td, J=11.6, 4.5 Hz, 1H), 3.70-3.62 (m, 1H), 3.65-3.51 (m, 2H), 3.51-3.40 (m, 1H), 1.84 (d, J=14.1 Hz, 1H), 1.76-1.67 (m, 1H), 1.63 (s, 1H), 1.58 (d, J=2.1 Hz, 2H), 1.49 (dddd, J=21.5, 16.0, 10.4, 5.6 Hz, 1H), 1.37-1.02 (m, 4H), 0.98 (d, J=6.7 Hz, 3H).

Compounds 28A-OMe*, 28B—OMe*, 28C—OMe*, and 28D-OMe* may be separated by chiral HPLC from rac-28-OMe of step 5.

### Example 29. Synthesis of 1-(4-chloro-5-fluoro-2-hydroxyphenyl)-N-(5-methyl-1-(1H-tetrazol-5-yl)azepan-3-yl)cyclopropane-1-carboxamide (Compound 29, rac-29), 1-(4-chloro-5-fluoro-2-methoxyphenyl)-N-(5-methyl-1-(1H-tetrazol-5-yl)azepan-3-yl)cyclopropane-1-carboxamide (Compound 29-OMe, rac-29-OMe), Compounds 29A*, 29B*, 29C*, 29D*, and Compounds 29A-OMe*, 29B—OMe*, 29C—OMe*, 29D-OMe*

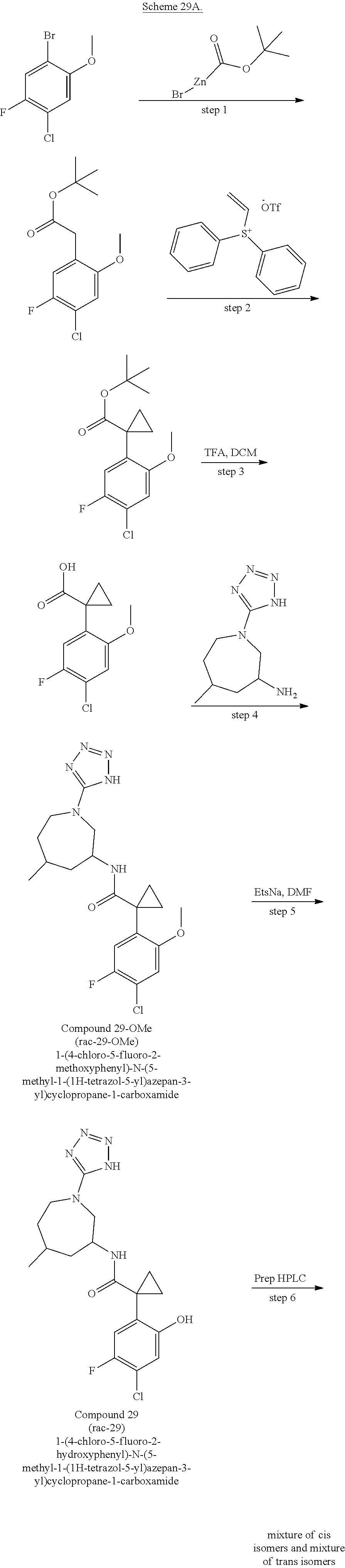

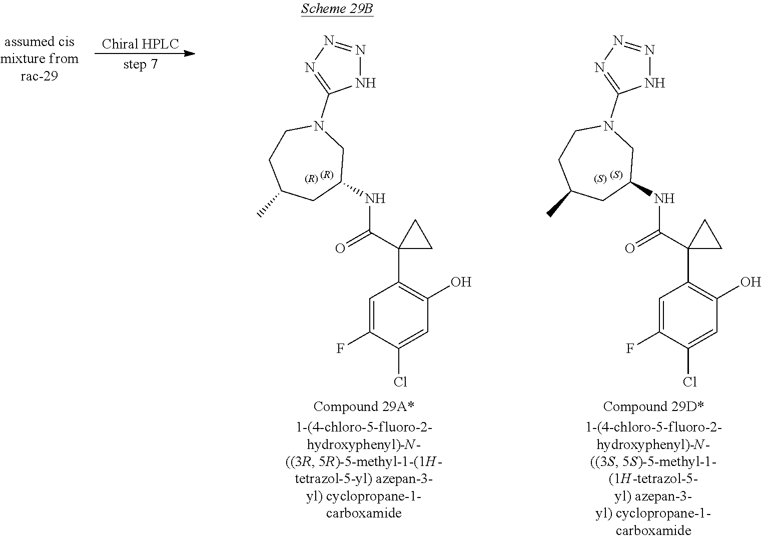

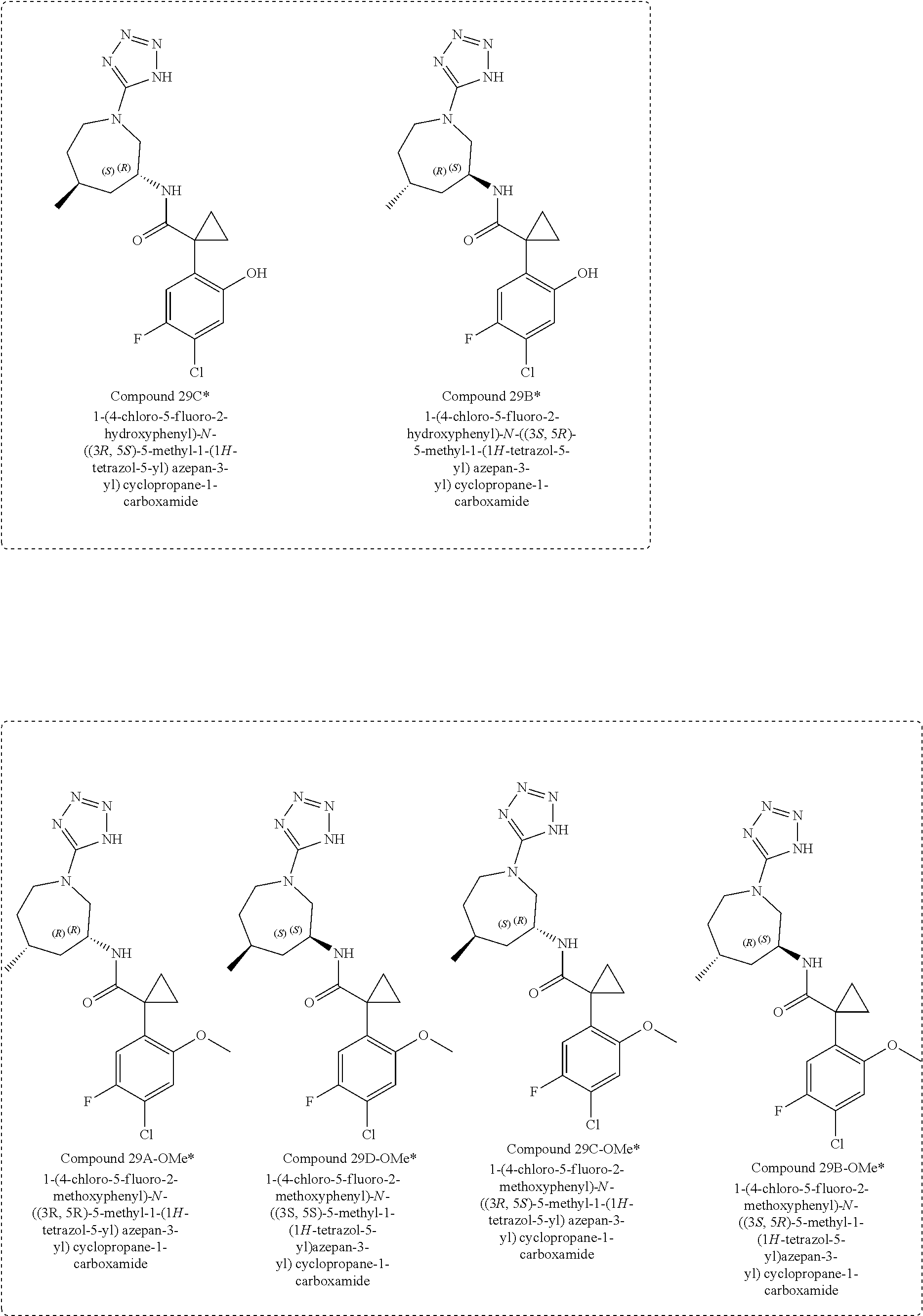

### Example 29 Follows Protocol A

Step 1: Into a 40 mL round-bottom flask was added 1-bromo-4-chloro-5-fluoro-2-methoxybenzene (200 mg, 0.835 mmol, 1 equiv) and tert-butyl 2-(bromozincio)acetate (435.03 mg, 1.670 mmol, 2 equiv), bis(tri-tert-butylphosphine)palladium(0) (Pd[(t-Bu)₃P]₂) (42.68 mg, 0.08 mmol, 0.10 equiv), tetrahydrofuran (THF) (2 mL) at room temperature. The resulting mixture was stirred for 2 h at 80° C. under nitrogen atmosphere. Desired product could be detected by GCMS. The residue was purified by silica gel column chromatography, eluting with petroleum ether:ethyl acetate (10:1) to provide tert-butyl 2-(4-chloro-5-fluoro-2-methoxyphenyl)acetate (150 mg, 65% yield).

Step 2: To a 25 mL round-bottom flask was added tert-butyl 2-(4-chloro-2,3-difluorophenyl)acetate (350 mg, 1.332 mmol, 1 equiv) and ethenyldiphenylsulfanium (543.55 mg, 2.548 mmol, 2 equiv), 1,8-diazabicyclo[5.4.0]undec-7-ene (DBIU) (581.88 mg, 3.822 mmol, 3 equiv), and dimethylsulfoxide (DMSO) (3 mL) at room temperature. The resulting mixture was stirred for 6 h at room temperature. Desired product could be detected by GCMS. The residue was purified by silica gel column chromatography, eluting with petroleum ether:ethyl acetate (10:1) to provide tert-butyl 1-(4-chloro-2,3-difluorophenyl)cyclopropane-1-carboxylate (160 mg, 42% yield).

Step 3: Into a 100 mL round-bottom flask was added tert-butyl 1-(4-chloro-5-fluoro-2-methoxyphenyl)cyclopropane-1-carboxylate (160 mg, 0.53 mmol, 1 equiv) and trifluoroacetic acid (TFA) (3 mL), dichloromethane (DCM) (6 mL) at room temperature. The mixture was stirred for 2 h at room temperature. Desired product could be detected by LCMS. The resulting mixture was concentrated under reduced pressure. This resulted in 1-(4-chloro-5-fluoro-2-methoxyphenyl)cyclopropane-1-carboxylic acid (140 mg, >100% yield). LCMS (ES, m/z): RT=0.88 min, m/z=243[M+1]⁻.

Step 4: Into a 40 mL round-bottom flask was added 1-(4-chloro-5-fluoro-2-methoxyphenyl)cyclopropane-1-carboxylic acid (“carboxyl (ii) reagent”) (140 mg, 0.57 mmol, 1 equiv), hydroxybenzotriazole (HOBT) (154.65 mg, 1.14 mmol, 2 equiv), 1-ethyl-3-(3-dimethylaminopropyl)carbodiimide (EDCI) (219.40 mg, 1.14 mmol, 2 equiv), diisopropylethyl amine (DIEA) (221.88 mg, 1.71 mmol, 3 equiv), dimethyl formamide (DMF) (2 mL) and 5-methyl-1-(1H-1,2,3,4-tetrazol-5-yl)azepan-3-amine (“amine (i) reagent”) (123.54 mg, 0.62 mmol, 1.1 equiv) at room temperature. The mixture was stirred for 1 h at room temperature. Desired product could be detected by LCMS. The residue was purified by reverse flash chromatography (C18 silica gel; mobile phase, acetonitrile (MeCN) in water, 10% to 50% gradient in 10 min; detector, UV 254 nm) to provide 1-(4-chloro-5-fluoro-2-methoxyphenyl)-N-(5-methyl-1-(1H-tetrazol-5-yl)azepan-3-yl)cyclopropane-1-carboxamide (Compound 29-OMe, rac-29-OMe) (150 mg, 62% yield) as a mixture of cis and trans isomers. LCMS (ES, m/z): RT=0.83 min, m/z=423[M+1]⁺. ¹H NMR (400 MHz, DMSO-d₆) δ 7.24 (d, J=17.6, 9.7 Hz, 1H), 7.13 (d, J=22.3, 6.4 Hz, 1H), 4.14 (q, J=5.9 Hz, 1H), 3.79 (s, 1H), 3.75 (s, 1H), 3.54-3.41 (m, 2H), 3.22 (d, J=9.0, 4.6 Hz, 2H), 3.06-2.92 (m, 1H), 2.28 (t, J=7.2 Hz, 1H), 2.19 (s, 2H), 1.72-1.64 (m, 1H), 1.58-1.21 (m, 3H), 1.09-0.94 (m, 4H), 0.87 (d, J=6.8 Hz, 2H).

Step 5: Into a 40 mL round-bottom flask was added Compound 29-OMe (100 mg, 0.02 mmol, 1 equiv), dimethyl formamide (DMF) (5 mL) and (ethylsulfanyl)sodium (596.70 mg, 0.72 mmol, 30 equiv) at room temperature. The mixture was stirred for 2 h at 80° C. Desired product could be detected by LCMS. The residue was purified by reverse flash chromatography (C18 silica gel; mobile phase, acetonitrile (MeCN) in water, 10% to 50% gradient in 30 min; detector, UV 254 nm) to provide crude product, 1-(4-chloro-5-fluoro-2-hydroxyphenyl)-N-(5-methyl-1-(1H-tetrazol-5-yl)azepan-3-yl)cyclopropane-1-carboxamide (Compound 29, rac-29) (60 mg), as a mixture of cis and trans isomers.

Step 6: The mixture of cis and trans isomers of rac-29 were separated using the following Prep conditions (XBridge Prep Phenyl OBD Column, 19*250 mm, 5 μm; Mobile Phase A: water (0.1% trifluoroacetic acid (TFA)), Mobile Phase B: acetonitrile (MeCN); Flow rate: 25 mL/min; Gradient: 35% B to 45% B in 10 min, 45% B; Wave Length: 254 nm; RT(min): 9) to provide an assumed mixture of cis isomers, Compounds 29A* and 29B* (22 mg, 18% yield). LCMS (ES, m/z): RT=0.74 min, m/z=409[M+1]⁺. Stereochemistry assigned arbitrarily. The assumed trans mixture of isomers, Compounds 29B* and 29C*, was not isolated.

Step 7: The assumed cis mixture of isomers from rac-29 (22 mg) was further separated by PREP-CHIRAL HPLC (Lux 5 μm Cellulose-4, 2.12*25 cm, 5 μm; Mobile Phase A: Hexanes (0.1% trifluoroacetic acid (TFA)), Mobile Phase B: isopropanol (IPA); Flow rate: 20 mL/min; Gradient: 30% B to 30% B in 13 min; Wave Length: 220/254 nm) to provide Compound 29A* (RT(min): 8.893, 4.5 mg) and Compound 29D* (RT(min): 12.083, 5.1 mg), stereochemistry arbitrarily assigned.

Compound 29A*: LCMS (ES, m/z): RT=1.38 min, m/z=409[M+1]⁺. ¹H NMR (400 MHz, DMSO-d₆) δ 9.92 (s, 1H), 7.08 (d, J=9.9 Hz, 1H), 6.85 (d, J=6.5 Hz, 1H), 6.54 (d, J=8.0 Hz, 1H), 4.14 (d, J=8.1, 5.0 Hz, 1H), 3.53 (d, J=13.5, 7.6, 4.2 Hz, 3H), 3.19 (d, J=13.9, 9.7, 4.4 Hz, 1H), 1.75 (d, J=6.3 Hz, 1H), 1.72-1.63 (m, 1H), 1.62-1.53 (m, 1H), 1.45 (d, J=14.5, 4.9 Hz, 1H), 1.36 (d, J=10.0, 6.7, 3.5 Hz, 1H), 1.26 (d, J=13.1, 8.6, 4.7 Hz, 2H), 0.97 (d, J=10.2, 6.6, 3.7 Hz, 1H), 0.87 (d, J=6.8 Hz, 3H), 0.79 (d, J=9.8, 6.7, 3.6 Hz, 1H).

Compound 29D*: LCMS (ES, m/z): RT=1.02 min, m/z=409[M+1]⁺. ¹H NMR (400 MHz, DMSO-d₆) δ 7.08 (d, J=10.0 Hz, 1H), 6.85 (d, J=6.5 Hz, 1H), 6.54 (d, J=8.1 Hz, 1H), 4.15 (q, J=6.4 Hz, 1H), 3.50 (d, J=5.8 Hz, 3H), 3.19 (d, J=13.9 Hz, 1H), 1.76 (d, J=7.6 Hz, 1H), 1.67 (d, J=14.6 Hz, 1H), 1.61-1.53 (m, 1H), 1.45 (q, J=14.4 Hz, 1H), 1.35 (t, J=10.8 Hz, 1H), 1.25 (t, J=14.2 Hz, 2H), 0.97 (d, J=10.3 Hz, 1H), 0.87 (d, J=6.8 Hz, 2H), 0.79 (d, J=9.7 Hz, 1H).

Compounds 29A-OMe*, 29B—OMe*, 29C—OMe*, and 29D-OMe* may be separated by chiral HPLC from rac-29-OMe of step 4.

### Example 30. Synthesis of 1-(4-(difluoromethyl)-2-hydroxyphenyl)-N-(5-methyl-1-(1H-tetrazol-5-yl)azepan-3-yl)cyclopropane-1-carboxamide (Compound 30, rac-30), 1-(4-(difluoromethyl)-2-methoxyphenyl)-N-(5-methyl-1-(1H-tetrazol-5-yl)azepan-3-yl)cyclopropane-1-carboxamide (Compound 30-OMe, rac-30-OMe), Compounds 30A*, 30B*, 30C*, and 30D*, and Compounds 30A-OMe*, 30B—OMe*, 30C—OMe*, and 30D-OMe*

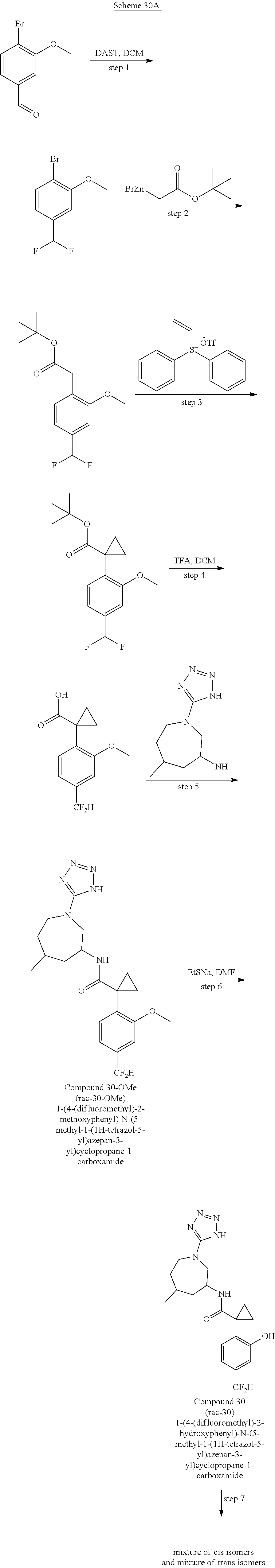

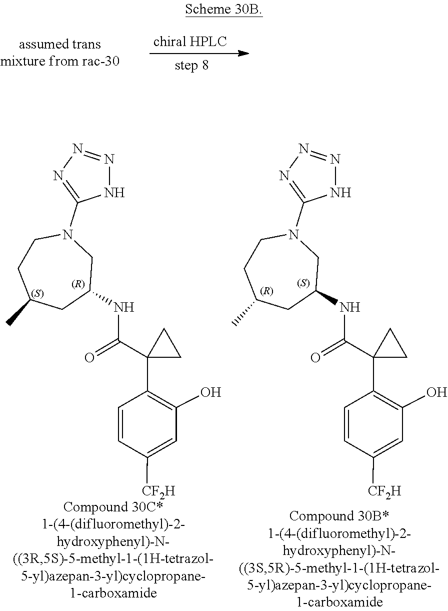

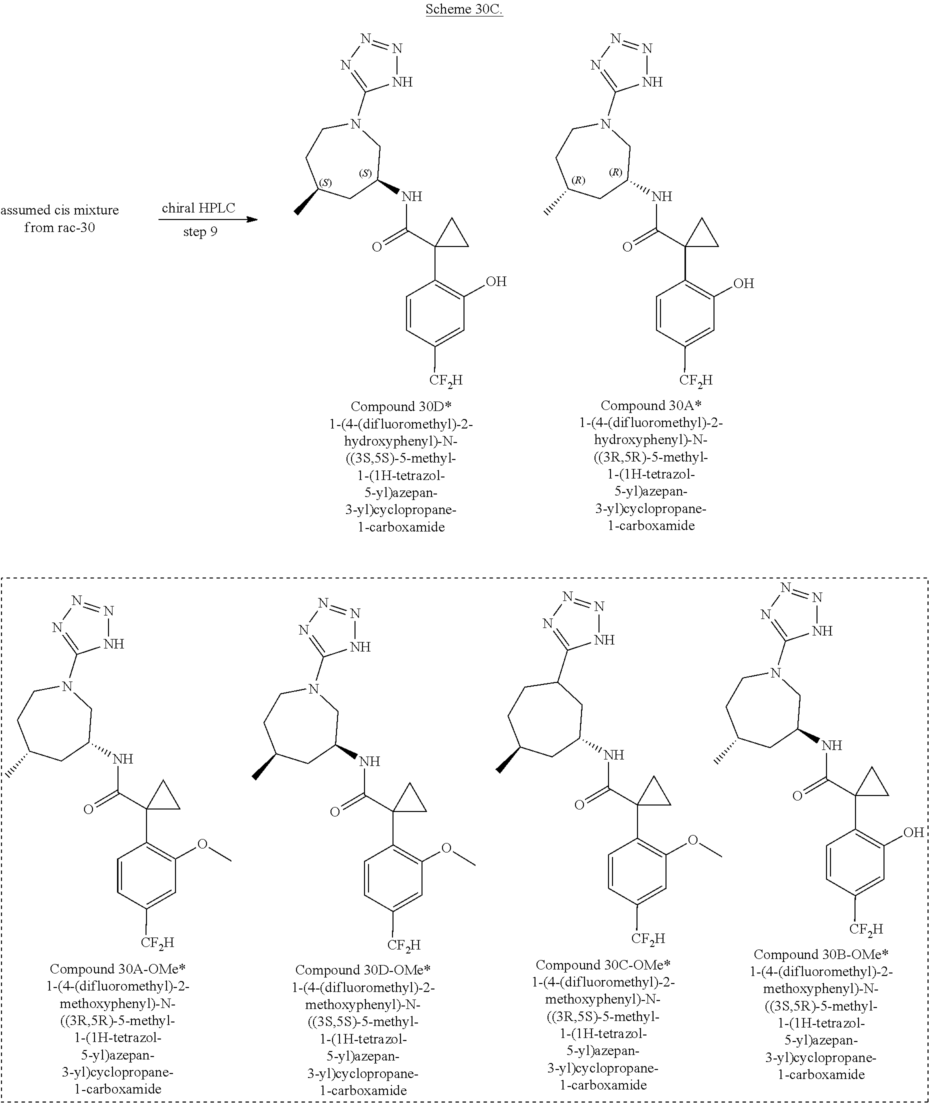

### Example 30 Follows Protocol A

Step 1: To a stirred solution of 4-bromo-3-methoxybenzaldehyde (1 g, 4.650 mmol, 1 equiv) in dichloromethane (DCM) (5 mL) was added diethylaminosulfur trifluoride (DAST) (3.75 g, 23.250 mmol, 5 equiv) dropwise at 0° C. The resulting mixture was stirred overnight at room temperature under air atmosphere. Desired product could be detected by GCMS. The reaction was quenched with ice water at 0° C. The aqueous layer was extracted with dichloromethane (DCM) (3×10 mL). The residue was purified by silica gel column chromatography, eluting with dichloromethane (DCM)/petroleum ether (1:5) to provide 1-bromo-4-(difluoromethyl)-2-methoxybenzene (1.03 g, 93% yield).

Step 2: Into an 8 mL vial was added 1-bromo-4-(difluoromethyl)-2-methoxybenzene (100 mg, 0.422 mmol, 1 equiv), tert-butyl 2-(bromozincio)acetate (329.61 mg, 1.266 mmol, 3 equiv), bis(tri-tert-butylphosphine)palladium(0) (Pd[(t-Bu)₃P]₂) (64.68 mg, 0.127 mmol, 0.3 equiv) and tetrahydrofuran (THF) (1 mL). The resulting mixture was stirred overnight at 60° C. under nitrogen atmosphere. Desired product could be detected by GCMS. The resulting residue was dried under vacuum. The residue was purified by Prep-TLC (petroleum ether:ethyl acetate 6:1) to provide tert-butyl 2-[4-(difluoromethyl)-2-methoxyphenyl]acetate (102 mg, 89% yield).

Step 3: Into a 20 mL vial was added tert-butyl 2-[4-(difluoromethyl)-2-methoxyphenyl]acetate (1.2 g, 4.407 mmol, 1 equiv), diphenylvinylsulfonium triflate (1.92 g, 5.288 mmol, 1.2 equiv), 1,8-diazabicyclo[5.4.0]undec-7-ene (DBU) (2.01 g, 13.221 mmol, 3 equiv) and dimethylsulfoxide (DMSO) (8 mL). The resulting mixture was stirred for 2 h at 40° C. under air atmosphere. Desired product could be detected by LCMS. The residue was purified by reverse flash chromatography (C18 silica gel; mobile phase, acetonitrile (MeCN):water=1:1) to provide tert-butyl 1-[4-(difluoromethyl)-2-methoxyphenyl]cyclopropane-1-carboxylate (330 mg, 25% yield).

Step 4: Into a 20 mL vial was added tert-butyl 1-[4-(difluoromethyl)-2-methoxyphenyl]cyclopropane-1-carboxylate (320 mg, 1.073 mmol, 1 equiv), trifluoroacetic acid (TFA) (1 mL, 13.463 mmol, 13 equiv) and dichloromethane (DCM) (5 mL). The resulting mixture was stirred for 2 h at room temperature under air atmosphere. Desired product could be detected by LCMS. The resulting residue was dried under vacuum to provide 1-[4-(difluoromethyl)-2-methoxyphenyl]cyclopropane-1-carboxylic acid (270 mg, 83% yield). The crude product was used in the next step directly without further purification.

Step 5: Into an 8 mL vial was added 1-[4-(difluoromethyl)-2-methoxyphenyl]cyclopropane-1-carboxylic acid (“carboxyl (ii) reagent”) (10 mg, 0.041 mmol, 1 equiv), hydroxybenzotriazole (HOBT) (178.52 mg, 1.322 mmol, 2 equiv), l-ethyl-3-(3-dimethylaminopropyl)carbodiimide (EDCI) (253.26 mg, 1.322 mmol, 2 equiv), diisopropylethyl amine (DIEA) (256.12 mg, 1.983 mmol, 3 equiv) and dimethyl formamide (DMF) (0.4 mL) for 30 min at room temperature. To the above mixture was added 5-methyl-1-(1H-1,2,3,4-tetrazol-5-yl)azepan-3-amine (“amine (i) reagent”) (155.57 mg, 0.793 mmol, 1.2 equiv) in portions over at room temperature. The resulting mixture was stirred for additional 2 h at room temperature. Desired product could be detected by LCMS. The residue was purified by reverse flash chromatography (C18 silica gel; mobile phase, acetonitrile (MeCN):water=1:1) to provide 1-(4-(difluoromethyl)-2-methoxyphenyl)-N-(5-methyl-1-(1H-tetrazol-5-yl)azepan-3-yl)cyclopropane-1-carboxamide (Compound 30-OMe, rac-30-OMe) (270 mg, 97% yield) as a mixture of cis and trans isomers.

Step 6: Into an 8 mL vial was added rac-30-OMe (150 mg, 0.357 mmol, 1 equiv), (ethylsulfanyl)sodium (450.12 mg, 5.355 mmol, 15 equiv) and dimethyl formamide (DMF) (2 mL). The resulting mixture was stirred for 2 h at 120° C. under air atmosphere. Desired product could be detected by LCMS. The resulting mixture was filtered, the filter cake was washed with ethyl acetate (EtOAc) (3×5 mL). The filtrate was concentrated under reduced pressure. The residue was purified by reverse flash chromatography (C18 silica gel; mobile phase, acetonitrile (MeCN):water=1:6) to provide 1-(4-(difluoromethyl)-2-hydroxyphenyl)-N-(5-methyl-1-(1H-tetrazol-5-yl)azepan-3-yl)cyclopropane-1-carboxamide (Compound 30, rac-30) as a mixture of cis and trans isomers.

Step 7: The crude product, rac-30, was purified by PREP HPLC (XBridge Prep Phenyl OBD Column, 19*150 mm, 5 μm; Mobile Phase A: water (10 mmol/L NH₄HCO₃), Mobile Phase B: acetonitrile (MeCN); Flow rate: 60 mL/min; Gradient: 13% B to 23% B in 8 min, 23% B to 28% B in 13 min, 28% B; Wave Length: 254 nm) to provide an assumed mixture of cis isomers (RT (min)=8.5, 15 mg, 10% yield) and an assumed mixture of trans isomers (RT (min)=13.0, 30 mg, 21% yield).

Step 8: The assumed mixture of trans isomers of rac-30 was separated by PREP-CHIRAL HPLC (CHIRALART Cellulose-SC, 2*25 cm, 5 μm; Mobile Phase A: Hexanes (0.1% TFA), Mobile Phase B: ethanol:dichloromethane=1:1; Flow rate: 20 mL/min; Gradient: 10% B to 10% B in 35 min; Wave Length: 254/220 nm) to provide Compound 30B* (RT (min)=28.41, 9.7 mg) and Compound 30C* (RT (min)=33.79, 9.9 mg, 5% yield). Stereochemistry arbitrarily assigned.

Compound 30B*: LCMS (ES, m/z): RT=1.00 min, m/z=407.2[M+H]⁺; ¹H NMR (400 MHz, DMSO-d₆) δ 14.56 (s, 1H), 9.98 (s, 1H), 7.15 (d, J=7.8 Hz, 1H), 7.05-6.74 (m, 3H), 6.31 (d, J=8.3 Hz, 1H), 4.22-4.02 (m, 1H), 3.55-3.47 (m, 3H), 3.20-3.06 (m, 1H), 1.65 (d, J=13.6 Hz, 2H), 1.60-1.51 (m, 1H), 1.51-1.36 (m, 2H), 1.30-1.19 (m, 2H), 0.98-0.90 (m, 1H), 0.87 (d, J=6.7 Hz, 3H), 0.83-0.75 (m, 1H).

Compound 30C*: LCMS (ES, m/z): RT=1.00 min, m/z=407.2[M+H]⁺; ¹H NMR (400 MHz, DMSO-d₆) δ 14.56 (s, 1H), 9.98 (s, 1H), 7.15 (d, J=7.8 Hz, 1H), 7.05-6.74 (m, 3H), 6.31 (d, J=8.3 Hz, 1H), 4.22-4.02 (m, 1H), 3.55-3.47 (m, 3H), 3.20-3.06 (m, 1H), 1.65 (d, J=13.6 Hz, 2H), 1.60-1.51 (m, 1H), 1.51-1.36 (m, 2H), 1.30-1.19 (m, 2H), 0.98-0.90 (m, 1H), 0.87 (d, J=6.7 Hz, 3H), 0.83-0.75 (m, 1H).

Step 9: The assumed mixture of cis isomers of rac-30 was separated by PREP-CHIRAL HPLC (CHIRAL ART Cellulose-SC, 2*25 cm, 5 μm; Mobile Phase A: Hexanes (0.1% trifluoroacetic acid (TFA)), Mobile Phase B: ethanol:dichloromethane=1:1; Flow rate: 20 mL/min; Gradient: 10% B to 10% B in 35 min; Wave Length: 254/220 nm) to provide Compound 30A* (RT(min): 28.42, 5.5 mg) and Compound 30D* (RT(min): 33.80, 6.1 mg). Stereochemistry arbitrarily assigned.

Compound 30A*: LCMS (ES, m/z): RT=0.74 min, m/z=407.2[M+H]⁺; ¹H NMR (400 MHz, DMSO-d₆) δ 9.95 (s, 1H), 7.29 (d, J=7.7 Hz, 1H), 7.11-6.74 (m, 4H), 3.98-3.91 (m, 1H), 3.59-3.52 (m, 1H), 3.52-3.42 (m, 1H), 3.41-3.31 (m, 2H), 1.80-1.70 (m, 1H), 1.50 (d, J=12.3 Hz, 2H), 1.41-1.24 (m, 3H), 1.23-1.15 (m, 1H), 0.95-0.82 (m, 5H).

Compound 30D*: LCMS (ES, m/z): RT=0.74 min, m/z=407.2[M+H]⁺; ¹H NMR (400 MHz, DMSO-d₆) δ 9.95 (s, 1H), 7.29 (d, J=7.7 Hz, 1H), 7.11-6.74 (m, 4H), 3.98-3.91 (m, 1H), 3.59-3.52 (m, 1H), 3.52-3.42 (m, 1H), 3.41-3.31 (m, 2H), 1.80-1.70 (m, 1H), 1.50 (d, J=12.3 Hz, 2H), 1.41-1.24 (m, 3H), 1.23-1.15 (m, 1H), 0.95-0.82 (m, 5H).

Compounds 30A-OMe*, 30B—OMe*, 30C—OMe*, and 30D-OMe* may be separated by chiral HPLC from rac-30-OMe of step 5.

### Example 31. Synthesis of 1-(3-fluoro-2-hydroxy-4-(trifluoromethyl)phenyl)-N-(5-methyl-1-(1H-tetrazol-5-yl)azepan-3-yl)cyclopropane-1-carboxamide (Compound 31, rac-31), 1-(3-fluoro-2-methoxy-4-(trifluoromethyl)phenyl)-N-(5-methyl-1-(1H-tetrazol-5-yl)azepan-3-yl)cyclopropane-1-carboxamide (Compound 31-OMe, rac-OMe), Compounds 31A*, 31B*, 31C*, and 31D*, and Compounds 31A-OMe*, 31B—OMe*, 31C—OMe*, and 31D-OMe*

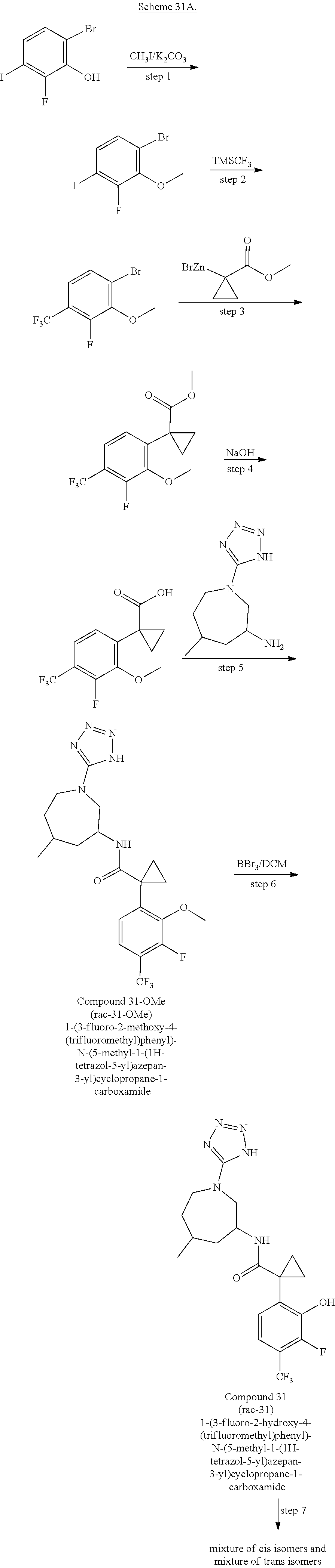

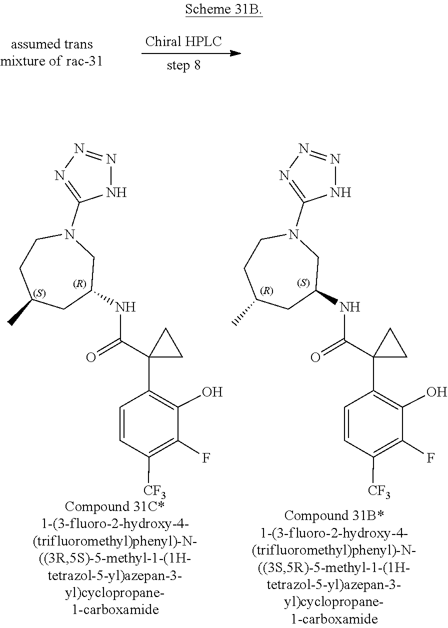

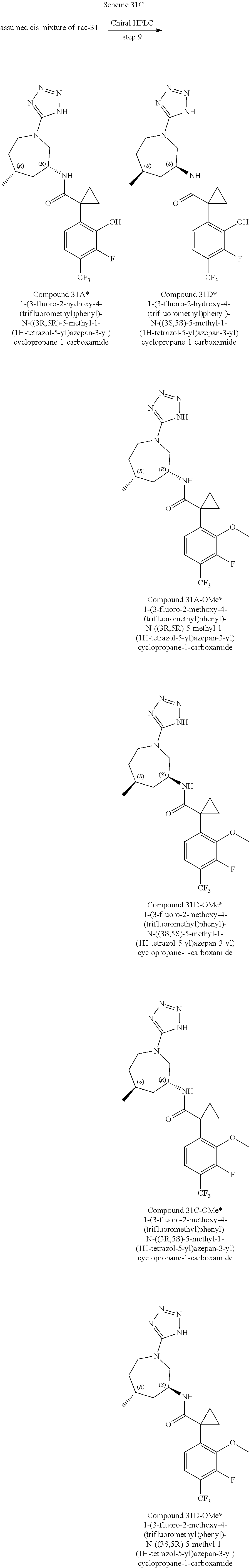

### Example 31 Follows Protocol A

Step 1: Into an 8 mL vial was added 6-bromo-2-fluoro-3-iodophenol (1 g, 3.156 mmol, 1 equiv), methyl iodide (0.54 g, 3.787 mmol, 1.2 equiv), potassium methaneperoxoate potassium (1.32 g, 9.468 mmol, 3 equiv) and dimethylformamide (DMF) (10 mL). The resulting mixture was stirred overnight at 60° C. under air atmosphere. Desired product could be detected by GCMS. The aqueous layer was extracted with ethyl acetate (EtOAc) (3×20 mL), the filtrate was concentrated under reduced pressure. The residue was purified by silica gel column chromatography, eluting with petroleum ether, to provide 1-bromo-3-fluoro-4-iodo-2-methoxybenzene (1.02 g, 98% yield).

Step 2: Into a 40 mL sealed tube was added 1-bromo-3-fluoro-4-iodo-2-methoxybenzene (1 g, 3.022 mmol, 1 equiv), CuI (1151.02 mg, 6.044 mmol, 2 equiv), KF (351.12 mg, 6.044 mmol, 2 equiv), trimethyl(trifluoromethyl)silane (TMSCF₃) (4296.95 mg, 30.220 mmol, 10 equiv) and dimethyl formamide (DMF) (8 mL). The resulting mixture was stirred overnight at 80° C. under nitrogen atmosphere. Desired product could be detected by GCMS. The resulting mixture was filtered, the filter cake was washed with ethyl acetate (EtOAc) (3×10 mL). The filtrate was concentrated under reduced pressure. The aqueous layer was extracted with ethyl acetate (EtOAc) (3×20 mL). The residue was purified by Prep-TLC (100% petroleum ether) to provide 1-bromo-3-fluoro-2-methoxy-4-(trifluoromethyl)benzene (540 mg, 65% yield).

Step 3: Into an 8 mL vial was added 1-bromo-3-fluoro-2-methoxy-4-(trifluoromethyl)benzene (260 mg, 0.952 mmol, 1 equiv), bromo[1-(methoxycarbonyl)cyclopropyl]zinc (2327.31 mg, 9.520 mmol, 10 equiv) and bis(tri-tert-butylphosphine)palladium(0) (Pd[(t-Bu)₃P]₂) (7 mL, 0.018 mmol, 0.5 equiv). The resulting mixture was stirred overnight at 60° C. under nitrogen atmosphere. Desired product could be detected by GCMS. The resulting mixture was filtered, the filter cake was washed with dichloromethane (DCM) (3×3 mL). The filtrate was concentrated under reduced pressure. The residue was purified by Prep-TLC (petroleum ether:ethyl acetate 7:1) to provide methyl 1-[3-fluoro-2-methoxy-4-(trifluoromethyl)phenyl]cyclopropane-1-carboxylate (170 mg, 61% yield).

Step 4: Into an 8 mL vial was added methyl 1-[3-fluoro-2-methoxy-4-(trifluoromethyl)phenyl]cyclopropane-1-carboxylate (160 mg, 0.548 mmol, 1 equiv), NaOH (65.70 mg, 1.644 mmol, 3 equiv), methanol (3.5 mL) and water (0.7 mL). The resulting mixture was stirred overnight at 40° C. under air atmosphere. Desired product could be detected by LCMS. The resulting oil was dried under vacuum. The mixture was acidified to pH 2 with conc. HCl to provide 1-[3-fluoro-2-methoxy-4-(trifluoromethyl)phenyl]cyclopropane-1-carboxylic acid (128 mg, 84% yield). The crude product was used in the next step directly without further purification.

Step 5: Into an 8 mL vial was added 1-[3-fluoro-2-methoxy-4-(trifluoromethyl)phenyl]cyclopropane-1-carboxylic acid (“carboxyl (ii) reagent”) (160 mg, 0.575 mmol, 1 equiv), hydroxybenzotriazole (HOBT) (155.43 mg, 1.150 mmol, 2 equiv), I-ethyl-3-(3-dimethylaminopropyl)carbodiimide (EDCI) (220.50 mg, 1.150 mmol, 2 equiv), diisopropylethyl amine (DIEA) (223 mg, 1.725 mmol, 3 equiv) and dimethyl formamide (DMF) (1.5 mL). To the above mixture was added 5-methyl-1-(1H-1,2,3,4-tetrazol-5-yl)azepan-3-amine (“amine (i) reagent”) (135.45 mg, 0.690 mmol, 1.2 equiv) in portions over 2 h at room temperature. Desired product could be detected by LCMS. The resulting residue was dried under vacuum, and then purified by reverse flash chromatography (C18 silica gel; mobile phase, acetonitrile (MeCN) in water=1:1) to provide 1-(3-fluoro-2-methoxy-4-(trifluoromethyl)phenyl)-N-(5-methyl-1-(1H-tetrazol-5-yl)azepan-3-yl)cyclopropane-1-carboxamide (Compound 31-OMe, rac-OMe) (118 mg, 45% yield), as a mixture of cis and trans isomers.

Step 6: To a stirred solution of Compound 31-OMe (100 mg, 0.219 mmol, 1 equiv) in dichloromethane (DCM) (3 mL) was added BBr₃ (0.6 mL, 6.347 mmol, 28.97 equiv) dropwise at 0° C. The resulting mixture was stirred for 2 h at room temperature under air atmosphere. Desired product could be detected by LCMS. The reaction was quenched by the addition of sat. sodium bicarbonate (aq.) (20 mL) at 0° C. The aqueous layer was extracted with ethyl acetate (EtOAc) (3×10 mL), dried, filtered, and concentrated to provide 1-(3-fluoro-2-hydroxy-4-(trifluoromethyl)phenyl)-N-(5-methyl-1-(1H-tetrazol-5-yl)azepan-3-yl)cyclopropane-1-carboxamide (Compound 31, rac-31), as a mixture of cis and trans isomers.

Step 7: The crude product, rac-31, was purified by PREP HPLC (XBridge Prep OBD C18 Column, 30*150 mm, 5 μm; Mobile Phase A: water (10 mmol/L NH₄HCO₃), Mobile Phase B: acetonitrile (MeCN); Flow rate: 60 mL/min; Gradient: 20% B to 28% B in 8 min, 28% B; Wave Length: 254 nm; RT(min): 6.78) to provide an assumed mixture of cis isomers as first eluting peak and assumed mixture of trans isomers as second eluting peak, stereochemistry arbitrarily assigned.

Step 9: The assumed trans mixture of rac-31 (20 mg) was purified by PREP-CHIRAL HPLC (Lux 5 μm Cellulose-4, 2.12*25 cm, 5 μm; Mobile Phase A: Hexanes (0.1% trifluoroacetic acid (TFA)), Mobile Phase B: isopropanol; Flow rate: 20 mL/min; Gradient: 30% B to 30% B in 11 min; Wave Length: 220/254 nm) to provide Compound 31B* (RT(min): 7.44, 7.10 mg) and Compound 31C* (RT(min): 10.80, 7.20 mg). Stereochemistry arbitrarily assigned.

Compound 31B*: LCMS:(ES, m/z):RT=0.73 min, m/z=443.2[M+H]⁺; ¹H NMR (400 MHz, Methanol-d4) δ 7.06 (d, J=8.2 Hz, 1H), 7.01-6.94 (m, 1H), 4.23-4.17 (m, 1H), 3.76-3.68 (m, 1H), 3.63-3.56 (m, 1H), 3.55-3.47 (m, 1H), 3.26-3.13 (m, 1H), 1.86-1.77 (m, 2H), 1.76-1.56 (m, 3H), 1.55-1.36 (m, 2H), 1.19-1.09 (m, 1H), 1.02-0.89 (m, 4H).

Compound 31C*: LCMS:(ES, m/z):RT=0.73 min, m/z=443.2[M+H]⁺; ¹H NMR (400 MHz, Methanol-d4) δ 7.06 (d, J=8.2 Hz, 1H), 7.01-6.94 (m, 1H), 4.23-4.17 (m, 1H), 3.76-3.68 (m, 1H), 3.63-3.56 (m, 1H), 3.55-3.47 (m, 1H), 3.26-3.13 (m, 1H), 1.86-1.77 (m, 2H), 1.76-1.56 (m, 3H), 1.55-1.36 (m, 2H), 1.19-1.09 (m, 1H), 1.02-0.89 (m, 4H).

Step 8: The assumed cis mixture of isomers from rac-31 was separated by PREP-CHIRAL HPLC (CHIRAL ART Cellulose-SB, 2*25 cm, 5 μm; Mobile Phase A: Hexanes (0.5% 2M NH₃-Methanol (MeOH)), Mobile Phase B: isopropanol (IPA): dichloromethane (DCM)=1:1; Flow rate: 20 mL/min; Gradient: 15% B to 15% B in 17 min; Wave Length: 220/254 nm) to provide Compound 31A* (RT(min): 15.65, 1.1 mg) and Compound 31D* (RT(min): 13.64, 1.2 mg). Stereochemistry arbitrarily assigned.

Compound 31A*: LCMS:(ES, m/z):RT=0.78 min, m/z=443.2[M+H]⁺; ¹H NMR (400 MHz, Methanol-d4) δ 7.21 (d, J=8.3 Hz, 1H), 7.16-7.10 (m, 1H), 4.03 (d, J=4.4 Hz, 1H), 3.65-3.58 (m, 1H), 3.55 (s, 1H), 3.53-3.40 (m, 2H), 1.84 (d, J=14.5 Hz, 1H), 1.71 (d, J=13.8 Hz, 1H), 1.65-1.55 (m, 3H), 1.53-1.41 (m, 1H), 1.34-1.18 (m, 1H), 1.15-1.05 (m, 2H), 0.98 (d, J=6.7 Hz, 3H).

Compound 31D*: LCMS:(ES, m/z):RT=0.78 min, m/z=443.2[M+H]⁺. ¹H NMR (400 MHz, Methanol-d4) δ 7.21 (d, J=8.3 Hz, 1H), 7.16-7.10 (m, 1H), 4.03 (d, J=4.4 Hz, 1H), 3.65-3.58 (m, 1H), 3.55 (s, 1H), 3.53-3.40 (m, 2H), 1.84 (d, J=14.5 Hz, 1H), 1.71 (d, J=13.8 Hz, 1H), 1.65-1.55 (m, 3H), 1.53-1.41 (m, 1H), 1.34-1.18 (m, 1H), 1.15-1.05 (m, 2H), 0.98 (d, J=6.7 Hz, 3H).

Compounds 31A-OMe*, 31B—OMe*, 31C—OMe*, and 31D-OMe* may be separated by chiral HPLC from rac-31-OMe of step 5.

### Example 32. Synthesis of 1-(4-cyclopropyl-3-fluoro-2-hydroxyphenyl)-N-(5-methyl-1-(1H-tetrazol-5-yl)azepan-3-yl)cyclopropane-1-carboxamide (Compound 32, rac-32), 1-(4-cyclopropyl-3-fluoro-2-methoxyphenyl)-N-(5-methyl-1-(1H-tetrazol-5-yl)azepan-3-yl)cyclopropane-1-carboxamide (Compound 32-OMe, rac-OMe), Compounds 32A*, 32B*, 32C*, and 32D*, and Compounds 32A-OMe*, 32B—OMe*, 32C—OMe*, and 32D-OMe*

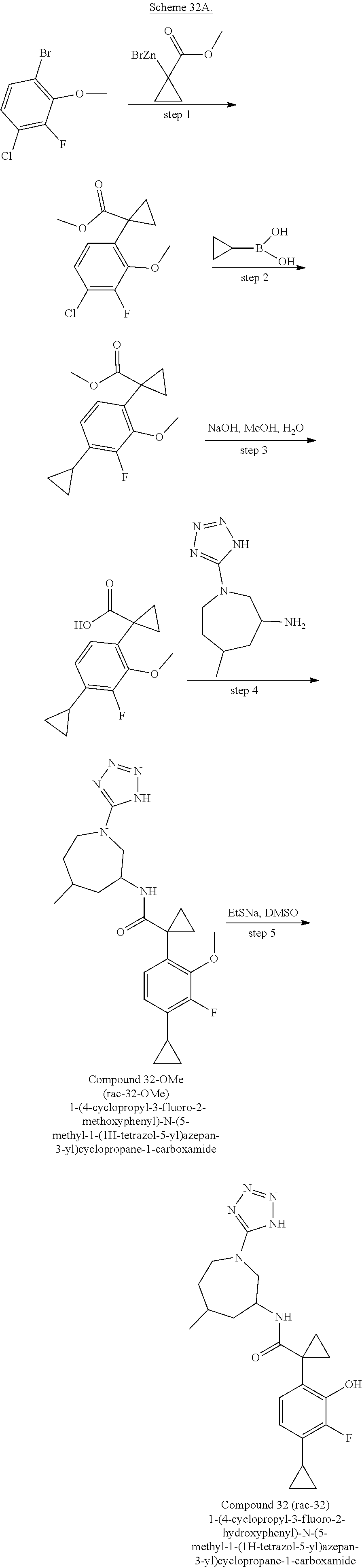

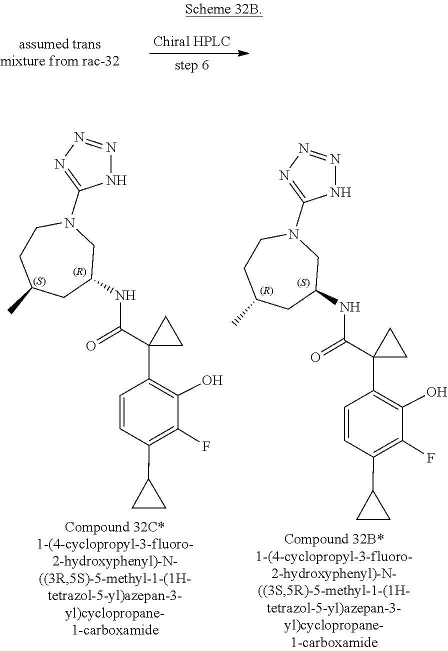

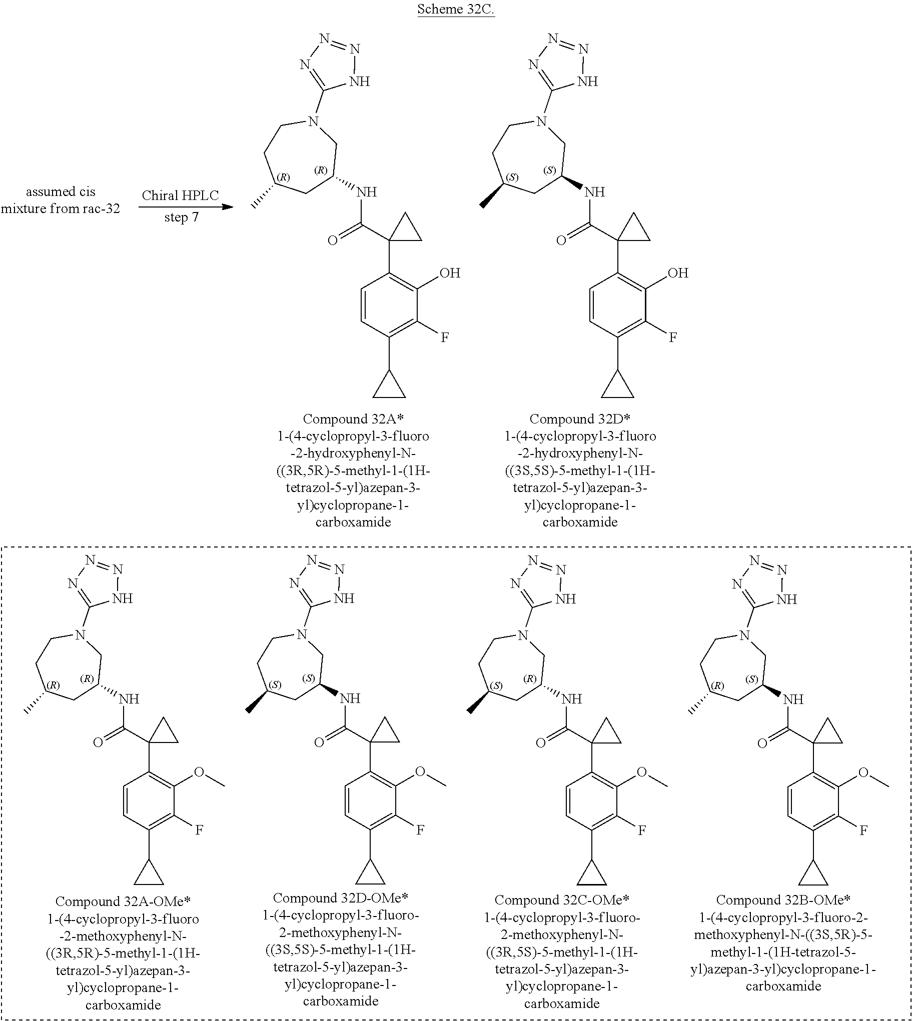

### Example 32 Follows Protocol A

Step 1: Into a 40 mL sealed tube was added 1-bromo-4-chloro-3-fluoro-2-methoxybenzene (500 mg, 2.09 mmol, 1 equiv), bromo[1-(methoxycarbonyl)cyclopropyl]zinc (0.93M in tetrahydrofuran (THF)) (10.46 ml, 10.44 mmol, 5 equiv), bis(tri-tert-butylphosphine)palladium(0) (Pd(P(t-Bu)₃)₂) (320.12 mg, 0.63 mmol, 0.30 equiv) and tetrahydrofuran (THF) (4 mL). The mixture was stirred for 2 h at 80° C. under nitrogen atmosphere. The reaction progress was monitored by GCMS. The reaction was quenched by the addition of water (5 mL) at room temperature. The aqueous layer was extracted with ethyl acetate (EtOAc) (3×5.00 mL). The organic phase was concentrated under reduced pressure. The residue was purified by reversed-phase flash chromatography (C18 silica gel; mobile phase, acetonitrile (MeCN) in water (0.10% formic acid), 10% to 50% gradient in 10 min; detector, UV 254 nm) to provide methyl 1-(4-chloro-3-fluoro-2-methoxyphenyl)cyclopropane-1-carboxylate (410 mg, 76% yield).

Step 2: Into a 40 mL sealed tube was added methyl 1-(4-chloro-3-fluoro-2-methoxyphenyl)cyclopropane-1-carboxylate (400 mg, 1.55 mmol, 1 equiv), cyclopropylboronic acid (265.67 mg, 3.09 mmol, 2 equiv), (2-Dicyclohexylphosphino-2′,4′,6′-triisopropyl-1,1′-biphenyl)[2-(2′-amino-1,1′-biphenyl)]palladium(II) methanesulfonate (XPhos Pd G3) (130.89 mg, 0.16 mmol, 0.1 equiv), Cs₂CO₃ (1511.51 mg, 4.64 mmol, 3 equiv), 1,4-dioxane (5 mL) and water (1 mL). The mixture was stirred for 2 h at 80° C. under nitrogen atmosphere. The reaction progress was monitored by GCMS. The reaction was quenched by the addition of water (5 mL) at room temperature. The aqueous layer was extracted with ethyl acetate (EtOAc) (3×5 mL). The resulting mixture was concentrated under reduced pressure. The residue was purified by reverse phase flash chromatography (C18 silica gel; mobile phase, acetonitrile (MeCN) in water (0.10% formic acid), 10% to 50% gradient in 10 min; detector, UV 254 nm) to provide methyl 1-(4-cyclopropyl-3-fluoro-2-methoxyphenyl)cyclopropane-1-carboxylate (250 mg, 61% yield).

Step 3: Into a 25 mL round-bottom flask was added methyl 1-(4-cyclopropyl-3-fluoro-2-methoxyphenyl)cyclopropane-1-carboxylate (250 mg, 0.95 mmol, 1 equiv), NaOH (189.17 mg, 4.73 mmol, 5 equiv), methanol (MeOH) (3 mL) and water (0.20 mL) at room temperature. The reaction progress was monitored by LCMS. The mixture was acidified to pH 6 with HCl(1M). The aqueous layer was extracted with ethyl acetate (EtOAc) (5×5 mL). This resulted in 1-(4-cyclopropyl-3-fluoro-2-methoxyphenyl)cyclopropane-1-carboxylic acid (200 mg, 84% yield). LCMS:(ES, m/z): RT=0.66 min, m/z=251 [M+H]⁺.

Step 4: Into a 25 mL round-bottom flask was added 1-(4-cyclopropyl-3-fluoro-2-methoxyphenyl)cyclopropane-1-carboxylic acid (“carboxyl (ii) reagent”) (200 mg, 0.78 mmol, 1 equiv), 5-methyl-1-(1H-1,2,3,4-tetrazol-5-yl)azepan-3-amine (“amine (i) reagent”) (172.52 mg, 0.89 mmol, 1.10equiv), (1-[bis(dimethylamino)methylene]-1H-1,2,3-triazolo[4,5-b]pyridinium 3-oxide hexafluorophosphate (HATU) (607.72 mg, 1.58 mmol, 2 equiv), diisopropylethyl amine (DIEA) (309.86 mg, 2.40 mmol, 3 equiv) and dimethyl formamide (DMF) (3 mL). The mixture was stirred for 1 h at room temperature. The reaction progress was monitored by LCMS. The residue was purified by reverse phase flash chromatography (C18 silica gel; mobile phase, acetonitrile (MeCN) in water (0.10% trifluoroacetic acid (TFA)), 10% to 50% gradient in 10 min; detector, UV 254 nm) to provide 1-(4-cyclopropyl-3-fluoro-2-methoxyphenyl)-N-(5-methyl-1-(1H-tetrazol-5-yl)azepan-3-yl)cyclopropane-1-carboxamide (Compound 32-OMe, rac-OMe) (100 mg, 29% yield) as a mixture of cis and trans isomers. LCMS:(ES, m/z): RT=0.61 min, m/z=429 [M+H]⁺. ¹H NMR (400 MHz, DMSO-d6) δ 7.22-7.13 (m, 1H), 6.84 (d, J=8.1 Hz, 1H), 6.42 (d, J=8.1 Hz, 1H), 3.95 (d, J=8.5 Hz, 1H), 3.84 (s, 3H), 3.57-3.48 (m, 4H), 2.10-1.95 (m, 1H), 1.75 (t, J=13.0 Hz, 1H), 1.50 (d, J=12.8 Hz, 2H), 1.41-1.14 (m, 5H), 0.95 (t, J=8.4 Hz, 2H), 0.91-0.78 (m, 5H), 0.71 (t, J=5.2, Hz, 2H).

Step 5: Into a 25 mL round-bottom flask was added Compound 32-OMe (90 mg, 0.21 mmol, 1 equiv), EtONa (142.93 mg, 2.10 mmol, 10 equiv), dimethylsulfoxide (DMSO) (3 mL). The mixture was stirred for 1 h at 100° C. The reaction progress was monitored by LCMS. The reaction was quenched by the addition of water (5 mL) at room temperature. The aqueous layer was extracted with EA (5×5 mL).

The residue was purified by reversed-phase flash chromatography (C18 silica gel; mobile phase, acetonitrile (MeCN) in water (0.10% trifluoroacetic acid (TFA)), 10% to 50% gradient in 10 min; detector, UV 254 nm) to provide 1-(4-cyclopropyl-3-fluoro-2-hydroxyphenyl)-N-(5-methyl-1-(1H-tetrazol-5-yl)azepan-3-yl)cyclopropane-1-carboxamide (Compound 32, rac-32) (80 mg, 92% yield) as a mixture of cis and trans isomers. LCMS:(ES, m/z): RT=0.66 min, m/z=415 [M+H]⁺.

Step 6: The crude product, rac-32 (80 mg), was purified by PREP HPLC (XBridge Prep Phenyl OBD Column, 19*150 mm, 5 μm; Mobile Phase A: water (10 mmol/L NH₄HCO₃), Mobile Phase B: acetonitrile (MeCN); Flow rate: 60 mL/min; Gradient: 18% B to 28% B in 9 min, 28% B; Wave Length: 254 nm; RT(min): 8.8) to provide an assumed mixture of trans isomers (25 mg, LCMS:(ES, m/z): RT=0.66 min, m/z=415 [M+H]+) as first eluting peak and an assumed mixture of cis isomers (10 mg, LCMS:(ES, m/z): RT=0.63 min, m/z=415 [M+H]+), as second eluting peak; stereochemistry arbitrarily assigned.

Step 7: The assumed trans mixture from rac-32 (25 mg) was separated by PREP-CHIRAL HPLC (Lux 5 μm Cellulose-4, 2.12*25 cm, 5 μm; Mobile Phase A: Hexanes (0.1% trifluoroacetic acid (TFA)), Mobile Phase B: isopropanol (IPA); Flow rate: 20 mL/min; Gradient: 50% B to 50% B in 10 min; Wave Length: 220/254 nm) to provide Compound 32B* (RT(min): 6.84, 4.5 mg) and Compound 32C* (RT(min): 9.25, 4.8 mg), stereochemistry arbitrarily assigned.

Compound 32B*: LCMS:(ES, m/z): RT=1.387 min, m/z=415[M+H]⁺. ¹H NMR (400 MHz, DMSO-d₆) δ 9.56 (s, 1H), 6.71 (d, J=8.0 Hz, 1H), 6.37 (d, J=8.2 Hz, 1H), 6.22 (t, J=7.5 Hz, 1H), 4.13 (t, J=9.0 Hz, 1H), 3.54 (d, J=5.5 Hz, 2H), 3.51-3.45 (m, 1H), 3.13 (t, J=17.8 Hz, 1H), 1.98 (t, J=8.6 Hz, 1H), 1.71-1.62 (m, 2H), 1.55 (t, J=12.9 Hz, 1H), 1.46 (d, J=14.4 Hz, 1H), 1.37 (t, J=10.0 Hz, 1H), 1.25 (t, J=10.4 Hz, 2H), 0.96-0.84 (m, 6H), 0.80-0.62 (m, 3H).

Compound 32C*: LCMS:(ES, m/z): RT=0.796 min, m/z=415[M+H]⁺. H NMR (400 MHz, DMSO-d6) δ 9.56 (s, 1H), 6.71 (d, J=8.0 Hz, 1H), 6.37 (d, J=8.2 Hz, 1H), 6.22 (t, J=7.5 Hz, 1H), 4.13 (t, J=9.0 Hz, 1H), 3.54 (d, J=5.5 Hz, 2H), 3.46-3.44 (m, 1H), 3.12 (t, J=17.8 Hz, 1H), 1.98 (t, J=8.6 Hz, 1H), 1.71-1.62 (m, 2H), 1.58-1.38 (m, 3H), 1.36-1.21 (m, 3H), 0.93 (d, J=4 Hz, 2H), 0.90-0.83 (m, 3H), 0.79-0.74 (m, 1H), 0.69 (t, J=8 Hz, 2H).

Step 8: The assumed cis mixture from rac-32 (10 mg) was separated by PREP-CHIRAL HPLC (Lux 5 μm Cellulose-4, 2.12*25 cm, 5 μm; Mobile Phase A: Hexanes (0.1% trifluoroacetic acid (TFA)), Mobile Phase B: isopropanol (IPA); Flow rate: 20 mL/min; Gradient: 30% B to 30% B in 23 min; Wave Length: 220/254 nm) to provide Compound 32A* (RT(min): 20.39, 0.7 mg) and Compound 32D* (RT(min): 15.65, 0.7 mg), stereochemistry arbitrarily assigned.

Compound 32A*: LCMS:(ES, m/z): RT=0.659 min, m/z=415 [M+H]⁺. ¹H NMR (400 MHz, DMSO-d₆) δ 9.50 (s, 1H), 6.91 (d, J=8.4 Hz, 1H), 6.86 (d, J=8.0 Hz, 1H), 6.36 (t, J=7.6 Hz, 1H), 3.95 (d, J=8.5 Hz, 1H), 3.55 (s, J=14.0 Hz, 4H), 2.10-1.95 (m, 1H), 1.75 (t, J=13.0 Hz, 1H), 1.50 (d, J=12.8 Hz, 2H), 1.41-1.14 (m, 5H), 0.95 (t, J=8.4 Hz, 2H), 0.91-0.78 (m, 5H), 0.71 (t, J=5.2, Hz, 2H).

Compound 32D*: LCMS:(ES, m/z): RT=0.796 min, m/z=415[M+H]⁺. H NMR (400 MHz, DMSO-d₆) δ 9.51 (s, 1H), 6.91 (d, J=8.4 Hz, 1H), 6.86 (d, J=8.0 Hz, 1H), 6.36 (t, J=7.5 Hz, 1H), 4.00-3.91 (m, 1H), 3.55 (d, J=14.1 Hz, 4H), 2.01 (t, J=8.5 Hz, 1H), 1.74 (t, J=13.2 Hz, 1H), 1.54-1.46 (m, 2H), 1.43-1.09 (m, 5H), 0.96 (t, J=3.6 Hz, 1H), 0.92-0.80 (m, 5H), 0.71 (t, J=4.8 Hz, 2H).

Compounds 32A-OMe*, 32B—OMe*, 32C—OMe*, and 32D-OMe* may be separated by chiral HPLC from rac-32-OMe of step 5.

### Example 33. Synthesis of 1-(4-chloro-2-hydroxy-6-methylphenyl)-N-((3R,5S)-5-methyl-1-(1H-tetrazol-5-yl)piperidin-3-yl)cyclopropane-1-carboxamide (Compound 33A) and 1-(4-chloro-2-methoxy-6-methylphenyl)-N-((3R,5S)-5-methyl-1-(1H-tetrazol-5-yl)piperidin-3-yl)cyclopropane-1-carboxamide (Compound 33A-OMe), and Compounds 33B, 33C, 33D, 33B—OMe, 33C—OMe, and 33D-OMe

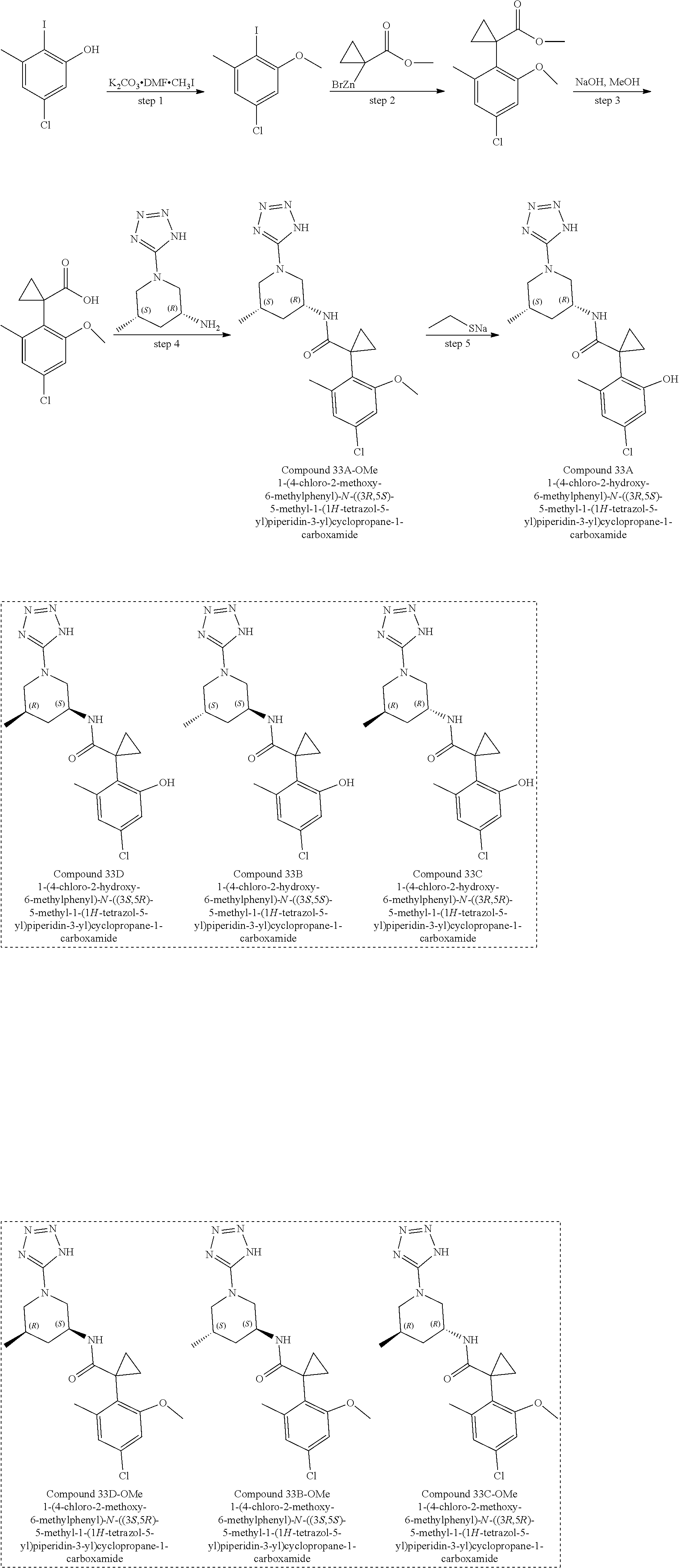

### Example 33 Follows Protocol A

Step 1: Into a 25 mL round-bottom flask was added 5-chloro-2-iodo-3-methylphenol (300 mg, 1.11 mmol, 1 equiv) and K₂CO₃ (463.29 mg, 3.35 mmol, 3 equiv), CH₃I (317.21 mg, 2.23 mmol, 2 equiv), and dimethyl formamide (DMF) (5 mL) at room temperature. The resulting mixture was stirred for 1 h at room temperature. The reaction progress was monitored by LCMS. The reaction was quenched by the addition of water (10 mL) at room temperature. The aqueous layer was extracted with ethyl acetate (EtOAc) (3×10 mL). The resulting mixture was concentrated under reduced pressure. The residue was purified by silica gel column chromatography, eluting with petroleum ether:ethyl acetate (9:1) to provide 5-chloro-2-iodo-1-methoxy-3-methylbenzene (300 mg, 95% yield).

Step 2: Into a 40 mL sealed tube was added 5-chloro-2-iodo-1-methoxy-3-methylbenzene (300 mg, 1.06 mmol, 1 equiv) and bromo[1-(methoxycarbonyl)cyclopropyl]zinc (3.47 mL, 3.18 mmol, 3 equiv), bis(tri-tert-butylphosphine)palladium(0) (Pd[(t-Bu)₃P]₂) (54.27 mg, 0.106 mmol, 0.1 equiv), and tetrahydrofuran (THF) (5 mL) at room temperature. The resulting mixture was stirred for 3 h at 80° C. under nitrogen atmosphere. The reaction progress was monitored by GCMS. The reaction was quenched by the addition of water (10 mL) at room temperature. The aqueous layer was extracted with ethyl acetate (EtOAc) (3×10 mL). The resulting mixture was concentrated under reduced pressure. The residue was purified by silica gel column chromatography, eluting with petroleum ether:ethyl acetate (10:1) to provide methyl 1-(4-chloro-2-methoxy-6-methylphenyl)cyclopropane-1-carboxylate (160 mg, 59% yield). GCMS: (ES, m/z): RT=6.21 min, m/z=254[M].

Step 3: Into a 50 mL vial was added methyl 1-(4-chloro-2-methoxy-6-methylphenyl)cyclopropane-1-carboxylate (150 mg 0.78 mmol 1 equiv), methanol (MeOH) (5 mL), and NaOH (117.75 mg, 3.92 mmol, 5 equiv) in water (1 mL) at room temperature. The resulting mixture was stirred for 1 h at room temperature. The reaction progress was monitored by LCMS. The mixture was acidified to pH 6 with HCl (1M). The aqueous layer was extracted with ethyl acetate (EtOAc) (3×10 mL). The resulting mixture was concentrated under reduced pressure. This resulted in 1-(4-chloro-2-methoxy-6-methylphenyl)cyclopropane-1-carboxylic acid (100 mg, 53% yield). LCMS (ES, m/z): RT=0.87 min, m/z=239[M−1]⁻.

Step 4: Into a 40 mL round-bottom flask was added 1-(4-chloro-2-methoxy-6-methylphenyl)cyclopropane-1-carboxylic acid (“carboxyl (ii) reagent”) (60 mg, 0.24 mmol, 1 equiv), (1-[bis(dimethylamino)methylene]-1H-1,2,3-triazolo[4,5-b]pyridinium 3-oxide hexafluorophosphate (HATU) (28.5 mg, 0.31 mmol, 1.3 equiv), diisopropylethyl amine (DIEA) (96.66 mg, 0.74 mmol, 3 equiv), dimethyl formamide (DMF) (4 mL) and (3R,5S)-5-methyl-1-(1H-1,2,3,4-tetrazol-5-yl)piperidin-3-amine (“amine (i) reagent”) (49.97 mg, 0.27 mmol, 1.10 equiv) at room temperature. The mixture was stirred for 2 h at room temperature. The reaction progress was monitored by LCMS. The residue was purified by reverse phase flash chromatography (C18 silica gel; mobile phase, acetonitrile (MeCN) in water, 10% to 50% gradient in 10 min; detector, UV 254 nm) to provide 1-(4-chloro-2-methoxy-6-methylphenyl)-N-((3R,5S)-5-methyl-1-(1H-tetrazol-5-yl)piperidin-3-yl)cyclopropane-1-carboxamide (Compound 33A-OMe) (30 mg, 30% yield). LCMS (ES, m/z): RT=0.89 min, m/z=405[M+1]⁺.

Step 5: Into a 25 mL round-bottom flask was added 1-(4-chloro-2-methoxy-6-methylphenyl)-N-[(3R,5S)-5-methyl-1-(1H-1,2,3,4-tetrazol-5-yl)piperidin-3-yl]cyclopropane-1-carboxamide (25 mg, 0.06 mmol, 1 equiv), dimethylsulfoxide (DMSO) (0.50 mL) and (ethylsulfanyl)sodium (155.80 mg, 1.86 mmol, 30 equiv) at room temperature. The mixture was stirred for 1 h at 100° C. The reaction progress was monitored by LCMS. The residue was purified by reverse phase flash chromatography (C18 silica gel; mobile phase, acetonitrile (MeCN) in water, 10% to 50% gradient in 10 min; detector, UV 254 nm) to provide 1-(4-chloro-2-hydroxy-6-methylphenyl)-N-((3R,5S)-5-methyl-1-(1H-tetrazol-5-yl)piperidin-3-yl)cyclopropane-1-carboxamide (Compound 33A) (20 mg). The crude product (20 mg) was purified by PREP HPLC (XBridge Shield RP18 OBD Column, 30*150 mm, 5 μm; Mobile Phase A: water (10 mmol/L NH₄HCO₃), Mobile Phase B: acetonitrile (MeCN); Flow rate: 60 mL/min; Gradient: 23% B to 35% B in 10 min, 35% B; Wave Length: 254 nm; RT(min): 8.5) to provide purified Compound 33A (1.90 mg). LCMS (ES, m/z): RT=0.74 min, m/z=391[M+1]⁺. ¹H NMR (400 MHz, DMSO-d₆) δ 9.88(bar, 1H), 6.73 (s, 1H), 6.24 (d, J=8.0 Hz, 1H), 3.80-3.73 (m, 3H), 2.55 (s, 3H), 2.24 (s, 3H), 2.00 (t, J=11.5 Hz, 1H), 1.63 (d, J=9.6 Hz, 2H), 1.48 (s, 2H), 0.92 (d, J=11.9 Hz, 1H), 0.83 (d, J=6.4 Hz, 3H).

Compound 33B—OMe and Compound 33B may be synthesized from (3S,5S)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 4 of Example 33.

Compound 33C—OMe and Compound 33C may be synthesized from (3R,5R)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 4 of Example 33.

Compound 33D-OMe and Compound 33D may be synthesized from (3S,5R)-5-methyl-1-(1H-1,2,3,4-tetrazol-5-yl)piperidin-3-amine instead of (3R,5S)-5-methyl-1-(1H-1,2,3,4-tetrazol-5-yl)piperidin-3-amine in step 4 of Example 33.

### Example 34. Synthesis of N-((1R,2S,5R)-8-(1H-tetrazol-5-yl)-8-azabicyclo[3.2.1]octan-2-yl)-1-(2-hydroxy-4-(trifluoromethyl)phenyl)cyclopropane-1-carboxamide (Compound 34B*), N-((1S,2R,5S)-8-(1H-tetrazol-5-yl)-8-azabicyclo[3.2.1]octan-2-yl)-1-(2-hydroxy-4-(trifluoromethyl)phenyl)cyclopropane-1-carboxamide (Compound 34C*), N-((1R,2S,5R)-8-(1H-tetrazol-5-yl)-8-azabicyclo[3.2.1]octan-2-yl)-1-(2-methoxy-4-(trifluoromethyl)phenyl)cyclopropane-1-carboxamide (Compound 34B—OMe*), N-((1S,2R,5S)-8-(1H-tetrazol-5-yl)-8-azabicyclo[3.2.1]octan-2-yl)-1-(2-methoxy-4-(trifluoromethyl)phenyl)cyclopropane-1-carboxamide (Compound 34C—OMe*), and Compounds 34A*, 34A-OMe*, 34D*, and 34D-OMe*

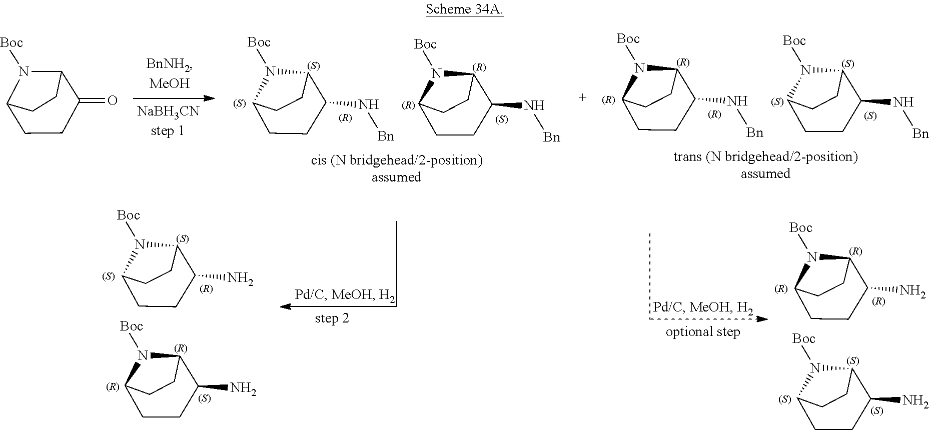

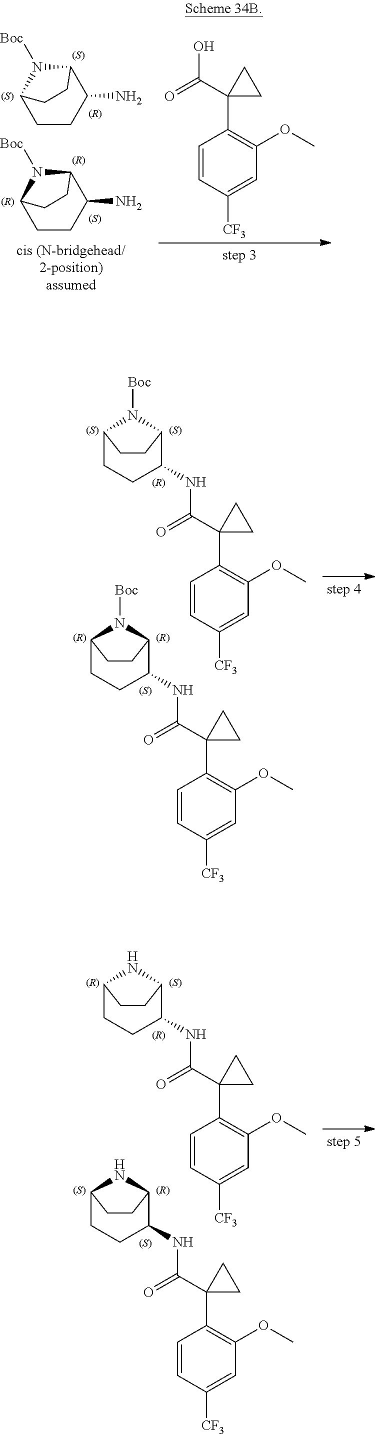

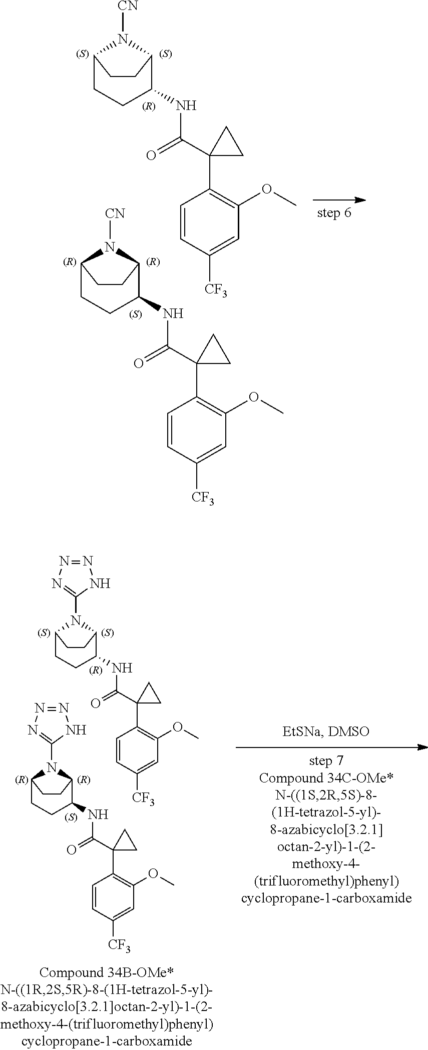

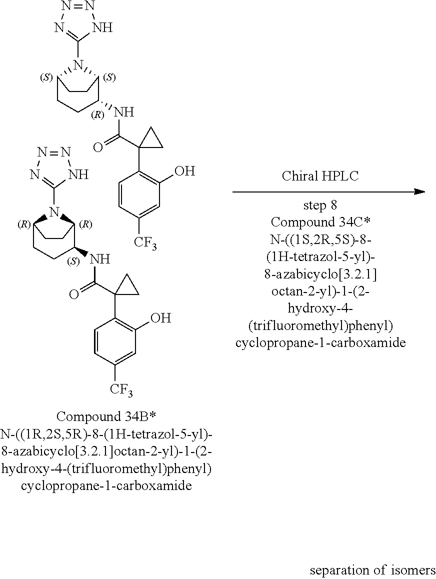

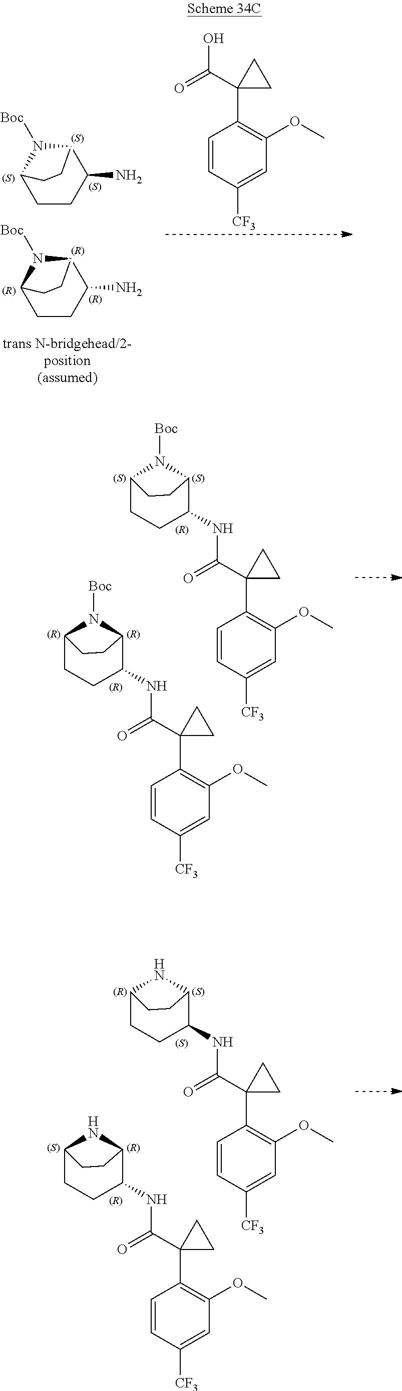

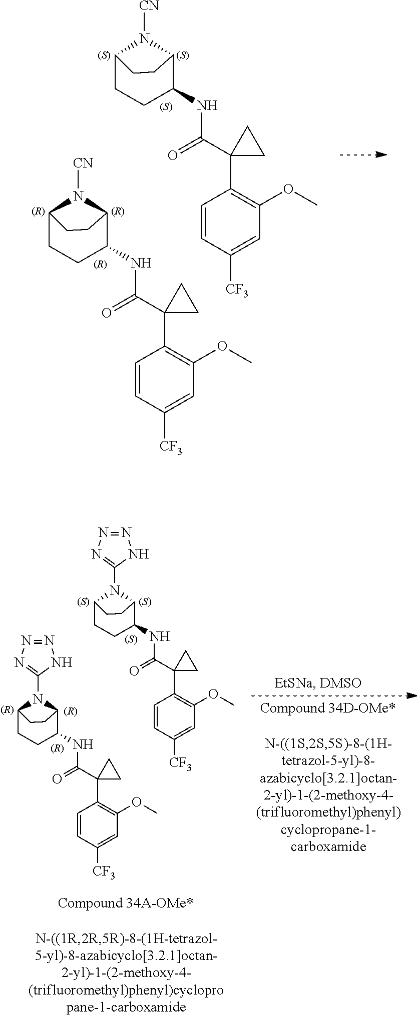

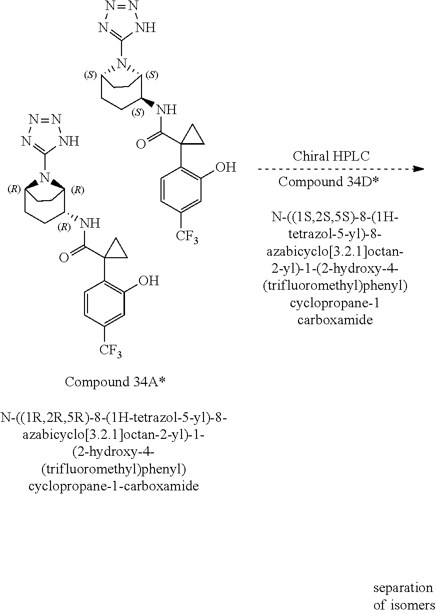

### Example 34Follows Protocol C

Step 1: Into a 40 mL vial was added tert-butyl 2-oxo-8-azabicyclo[3.2.1]octane-8-carboxylate (1000 mg, 4.43 mmol, 1 equiv) and benzylamine (951.28 mg, 8.87 mmol, 2 equiv) in methanol (MeOH) (12 mL) at room temperature. The resulting mixture was stirred for 14 h at 60° C. To the above mixture was added NaBH₃CN (836.80 mg, 13.31 mmol, 3 equiv) by batches at 0° C. The resulting mixture was stirred for an additional 1 h at 60° C. The reaction was monitored by LCMS. The reaction was quenched by the addition of water (5 mL) at 0° C. The resulting mixture was extracted with ethyl acetate (EtOAc) (3×30 mL). The combined organic layers were washed with brine (1×30 mL), dried over anhydrous Na₂SO₄.

After filtration, the filtrate was concentrated under reduced pressure. The residue was purified by silica gel column chromatography, eluting with petroleum ether/dichloromethane (DCM) (1:10). The crude product (1 g, 80% purity) was purified by PREP HPLC (XSelect CSH Prep C18 OBD Column, 19*250 mm, 5 μm; Mobile Phase A: water (10 mmol/L NH₄HCO₃), Mobile Phase B: acetonitrile (MeCN); Flow rate: 20 mL/min; Gradient: 60% B to 65% B in 8 min, 65% B; Wave Length: 254 nm; RT(min): 10) to provide tert-butyl (1S,2R,5S)-2-amino-8-azabicyclo[3.2.1]octane-8-carboxylate (relative, cis N-bridgehead/2-position) (600 mg, 95% purity, 43% yield) and tert-butyl (1R,2S,5R)-2-amino-8-azabicyclo[3.2.1]octane-8-carboxylate (relative, cis N-bridgehead/2-position) (60 mg, 30% purity, 4% yield), stereochemistry arbitrarily assigned. LCMS (ESI) (relative, trans): RT=1.24 min, m/z=317.2 [M+H]⁺.

Step 2: To a stirred solution of a mixture of SRS and RSR isomers of tert-butyl 2-(benzylamino)-8-azabicyclo[3.2.1]octane-8-carboxylate (relative, cis N-bridgehead/2-position) (500 mg, 1.58 mmol, 1 equiv) in methanol (MeOH) (10 mL) was added Pd/C (336.30 mg, 3.16 mmol, 2 equiv) at room temperature under hydrogen atmosphere. The resulting mixture was stirred for 16 h at room temperature. The reaction was monitored by LCMS. The resulting mixture was filtered, the filter cake was washed with methanol (MeOH) (3×10 mL), and the filtrate concentrated under reduced pressure to provide a mixture of SRS and RSR isomers of tert-butyl 2-amino-8-azabicyclo[3.2.1]octane-8-carboxylate (relative, cis N-bridgehead/2-position) (340 mg, 90% purity, 95% yield). LCMS (ESI): RT=1.68 min, m/z=227.2 [M+H]⁺.

Step 3: Into a 40 mL round-bottom flask was added 1-[2-methoxy-4-(trifluoromethyl)phenyl]cyclopropane-1-carboxylic acid (“carboxyl (ii) reagent”) (100 mg, 0.38 mmol, 1 equiv), triethylamine (TEA) (77.78 mg, 0.76 mmol, 2 equiv), dimethyl formamide (DMF) (2 mL) and a mixture of SRS and RSR isomers of tert-butyl 2-amino-8-azabicyclo[3.2.1]octane-8-carboxylate (“amine (iv) reagent”) (relative, cis N-bridgehead/2-position) (104.37 mg, 0.46 mmol, 1.2 equiv) at room temperature. Desired product could be detected by LCMS. The residue was purified by reverse flash chromatography (C18 silica gel; mobile phase, acetonitrile (MeCN) in water, 10% to 50% gradient in 10 min; detector, UV 254 nm) to provide a mixture of SRS and RSR isomers of tert-butyl 2-{1-[2-methoxy-4-(trifluoromethyl)phenyl]cyclopropaneamido}-8-azabicyclo[3.2.1]octane-8-carboxylate (relative, cis N-bridgehead/2-position) (80 mg, 44% yield). LCMS (ES, m/z): RT=1.09 min, m/z=469[M+1]⁺.

Step 4: Into a 40 mL round-bottom flask was added 1-[2-methoxy-4-(trifluoromethyl)phenyl]cyclopropane-1-carboxylic acid (100 mg, 0.38 mmol, 1 equiv), triethylamine (TEA) (77.78 mg, 0.76 mmol, 2 equiv), dimethyl formamide (DMF) (2 mL) and a mixture of SRS and RSR isomers of tert-butyl 2-amino-8-azabicyclo[3.2.1]octane-8-carboxylate (relative, cis N-bridgehead/2-position) (104.37 mg, 0.46 mmol, 1.2 equiv) at room temperature. Desired product could be detected by LCMS. The residue was purified by reverse flash chromatography (C18 silica gel; mobile phase, acetonitrile (MeCN) in water, 10% to 50% gradient in 10 min; detector, UV 254 nm) to provide a mixture of SRS and RSR isomers of N-[8-azabicyclo[3.2.1]octan-2-yl]-1-[2-methoxy-4-(trifluoromethyl)phenyl]cyclopropane-1-carboxamide (relative, cis N-bridgehead/2-position) (80 mg, 44% yield). LCMS (ES, m/z): RT=0.64 min, m/z=369[M+1]+. Stereochemistry arbitrarily assigned.

Step 5: Into a 40 mL round-bottom flask was added a mixture of SRS and RSR isomers of N-[8-azabicyclo[3.2.1]octan-2-yl]-1-[2-methoxy-4-(trifluoromethyl)phenyl]cyclopropane-1-carboxamide (relative, cis N-bridgehead/2-position) (75 mg, 0.20 mmol, 1 equiv) and BrCN (32.35 mg, 0.30 mmol, 1.5 equiv), and acetonitrile (MeCN) (2 mL) at room temperature. Desired product could be detected by LCMS. The residue was purified by silica gel column chromatography, eluting with petroleum ether:ethyl acetate (8:1) to provide a mixture of SRS and RSR isomers of N-[8-cyano-8-azabicyclo[3.2.1]octan-2-yl]-1-[2-methoxy-4-(trifluoromethyl)phenyl]cyclopropane-1-carboxamide (relative, cis N-bridgehead/2-position) (72 mg, 90% yield). LCMS (ES, m/z): RT=0.96 min, m/z=394[M+1]⁺.

Step 6: Into a 40 mL round-bottom flask was added a mixture of SRS and RSR isomers of N-[8-cyano-8-azabicyclo[3.2.1]octan-2-yl]-1-[2-methoxy-4-(trifluoromethyl)phenyl]cyclopropane-1-carboxamide (relative, cis N-bridgehead/2-position) (72 mg, 0.18 mmol, 1 equiv), (TMS)₃N (85.50 mg, 0.36 mmol, 2 equiv), NH₄Cl (29.37 mg, 0.54 mmol, 3 equiv), and dimethyl formamide (DMF) (0.50 mL) at room temperature. The mixture was stirred for 2 h at 100° C. under. Desired product could be detected by LCMS. The residue was purified by reverse flash chromatography (C18 silica gel; mobile phase, acetonitrile (MeCN) in water, 10% to 50% gradient in 10 min; detector, UV 254 nm) to provide a mixture of N-((1R,2S,5R)-8-(1H-tetrazol-5-yl)-8-azabicyclo[3.2.1]octan-2-yl)-1-(2-methoxy-4-(trifluoromethyl)phenyl)cyclopropane-1-carboxamide (Compound 34B—OMe*), and N-((1S,2R,5S)-8-(1H-tetrazol-5-yl)-8-azabicyclo[3.2.1]octan-2-yl)-1-(2-methoxy-4-(trifluoromethyl)phenyl)cyclopropane-1-carboxamide (Compound 34C—OMe*) (70 mg, 88% yield). LCMS (ES, m/z): RT=0.88 min, m/z=437[M+1]⁺.

Step 7: Into a 40 mL round-bottom flask was added the mixture of Compounds 34B—OMe* and 34C—OMe* (70 mg, 0.16 mmol, 1 equiv), dimethylsulfoxide (DMSO) (7 mL) and (ethylsulfanyl)sodium (404.71 mg, 4.80 mmol, 30 equiv) at room temperature. The mixture was stirred for 2 h at 80° C. Desired product could be detected by LCMS. The residue was purified by reverse flash chromatography (C18 silica gel; mobile phase, acetonitrile (MeCN) in water, 10% to 50% gradient in 10 min; detector, UV 254 nm) to provide crude mixture of N-((1R,2S,5R)-8-(1H-tetrazol-5-yl)-8-azabicyclo[3.2.1]octan-2-yl)-1-(2-hydroxy-4-(trifluoromethyl)phenyl)cyclopropane-1-carboxamide (Compound 34B*) and N-((1S,2R,5S)-8-(1H-tetrazol-5-yl)-8-azabicyclo[3.2.1]octan-2-yl)-1-(2-hydroxy-4-(trifluoromethyl)phenyl)cyclopropane-1-carboxamide (Compound 34C*) (40 mg). The crude product (40 mg) was purified by PREP HPLC (YMC-Actus Triart C18 ExRS, 30*150 mm, 5 μm; Mobile Phase A: water (10 mmol/L NH₄HCO₃), Mobile Phase B: acetonitrile (MeCN); Flow rate: 60 mL/min; Gradient: 15% B to 26% B in 10 min, 26% B; Wave Length: 254 nm; RT(min): 11.2) to provide a purified mixture of Compounds 34B* and 34C* (26 mg, 38% yield). LCMS (ES, m/z): RT=0.75 min, m/z=423[M+1]⁺.

Step 8: The purified mixture of Compounds 34B* and 34C* (26 mg) was separated by PREP-CHIRAL HPLC (YMC-Actus Triart C18 ExRS, 30*150 mm, 5 μm; Mobile Phase A: water (10 mmol/L NH₄HCO₃), Mobile Phase B: acetonitrile (MeCN); Flow rate: 60 mL/min; Gradient: 15% B to 26% B in 10 min, 26% B; Wave Length: 254 nm) to provide Compound 34C* (8.10 mg) as the first eluting peak, and Compound 34B* (6.30 mg) as the second eluting peak. Stereochemistry arbitrarily assigned.

Compound 34C*: LCMS (ES, m/z): RT=0.78 mm, m/z=423[M+1]⁺. ¹H NMR (400 MHz, DMSO-d₆) δ 10.30 (s, 1H), 7.39 (d, J=8.2 Hz, 1H), 7.12 (d, J=5.6 Hz, 2H), 6.38 (d, J=7.9 Hz, 1H), 4.15-4.05 (m, 2H), 3.84 (s, 1H), 1.89-1.82 (m, 1H), 1.78-1.64 (m, 3H), 1.62-1.49 (m, 2H), 1.48-1.40 (m, 3H), 1.35 (d, J=5.2 Hz, 1H), 0.99 (d, J=6.5 Hz, 1H), 0.88 (s, 1H).

Compound 34B*: LCMS (ES, m/z): RT=1.34 min, m/z=423[M+1]⁺. ¹H NMR (400 MHz, DMSO-d₆) δ 10.30 (s, 1H), 7.39 (d, J=8.1 Hz, 1H), 7.12 (d, J=5.6 Hz, 2H), 6.38 (d, J=7.9 Hz, 1H), 4.10 (d, J=17.9, 6.3 Hz, 2H), 1.89-1.79 (m, 1H), 1.77-1.62 (m, 3H), 1.61-1.51 (m, 2H), 1.48-1.40 (m, 3H), 1.35 (t, J=5.3 Hz, 1H), 0.99 (d, J=6.4, 4.2 Hz, 1H), 0.88 (d, J=6.8 Hz, 1H).

Compound 34A*, 34A-OMe*, 34D*, and 34D-OMe* may be prepared following the above Example 34, but using a mixture of RRR and SSS isomers of tert-butyl 2-(benzylamino)-8-azabicyclo[3.2.1]octane-8-carboxylate (relative, trans N-bridgehead/2-position), as shown in Scheme 34C.

### Example 35. 1-(4-(difluoromethoxy)phenyl)-N-((1R,4R,5S)-4-methyl-8-(1H-tetrazol-5-yl)-8-azabicyclo[3.2.1]octan-2-yl)cyclopropane-1-carboxamide (Compound 35′*), 1-(4-(difluoromethoxy)phenyl)-N-((1S,4S,5R)-4-methyl-8-(1H-tetrazol-5-yl)-8-azabicyclo[3.2.1]octan-2-yl)cyclopropane-1-carboxamide (Compound 35″*), 1-(4-(difluoromethoxy)phenyl)-N-((1R,4S,5S)-4-methyl-8-(1H-tetrazol-5-yl)-8-azabicyclo[3.2.1]octan-2-yl)cyclopropane-1-carboxamide (Compound 35′″*), 1-(4-(difluoromethoxy)phenyl)-N-((1S,4R,5R)-4-methyl-8-(1H-tetrazol-5-yl)-8-azabicyclo[3.2.1]octan-2-yl)cyclopropane-1-carboxamide (Compound 35″″*), and Compounds 35A*, 35B*, 35C*, 35D*, 35E*, 35F*, 35G*, and 35H*

### Part 1—Synthesis of Compound 35′*, 35″*, 35A*, 35B*, 35C* and 35D*

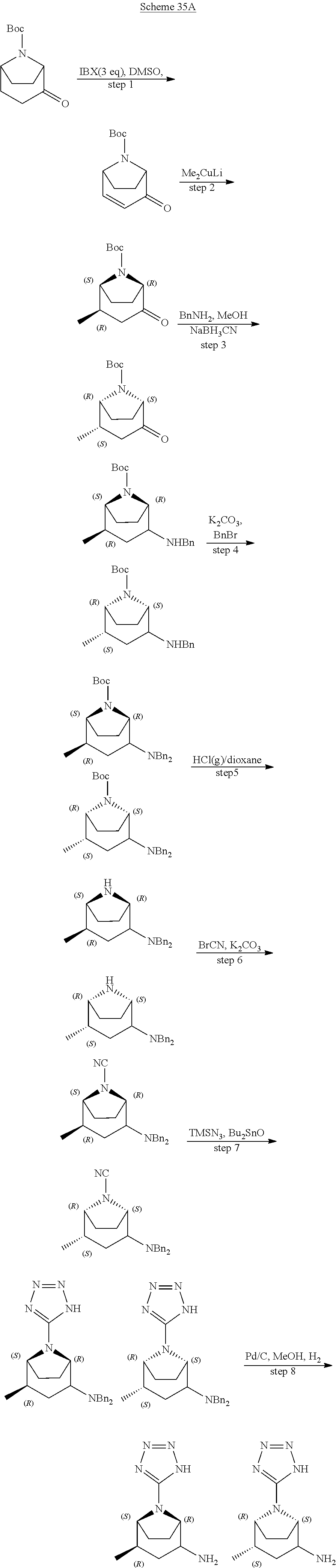

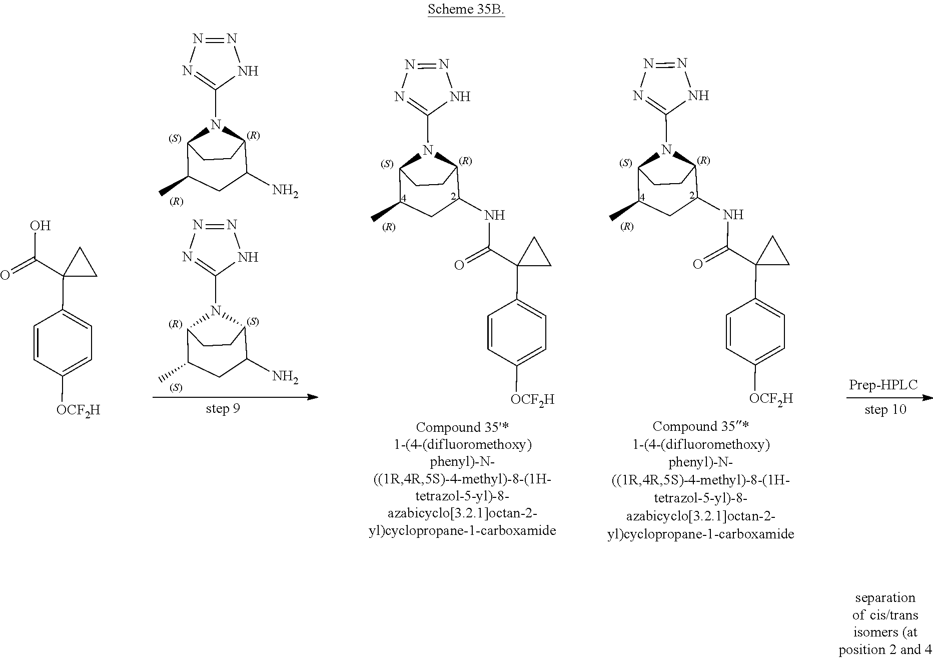

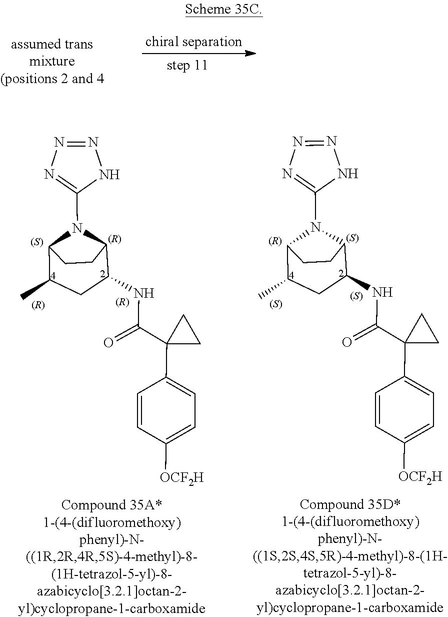

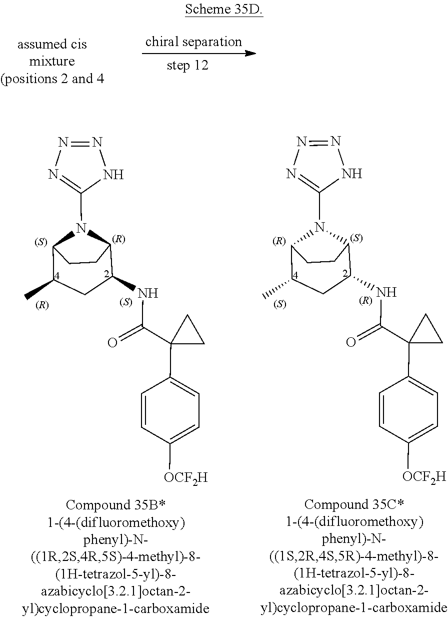

### Example 35 Follows Protocol A

Step 1: Into a 20 mL vial was added tert-butyl 2-oxo-8-azabicyclo[3.2.1]octane-8-carboxylate (600 mg, 2.66 mmol, 1 equiv) and 2-iodoxybenzoic acid (IBX) (2240 mg, 7.98 mmol, 3 equiv) in dimethyl sulfoxide (DMSO) (7 mL) at room temperature. The resulting mixture was stirred for 16 h at 80° C. The reaction was monitored by LCMS until completion. The reaction was repeated twenty-four times, and the batches combined and the volatiles were removed under reduced pressure. The residue was then purified by reversed-phase flash chromatography (C18 silica gel; mobile phase, acetonitrile (MeCN) in water, 30% to 40% gradient in 10 min; detector, UV 254 nm) to provide tert-butyl 4-oxo-8-azabicyclo[3.2.1]oct-2-ene-8-carboxylate (6 g, 41% yield). LCMS: (ES, m/z) RT=0.77 min, m/z=168.1 [M+H]⁺.

Step 2: To a stirred solution of tert-butyl 4-oxo-8-azabicyclo[3.2.1]oct-2-ene-8-carboxylate (1000 mg, 4.47 mmol, 1 equiv) in tetrahydrofuran (15 mL) was added lithium dimethyl cuprate (Me₂CuLi) (0.5 M in Et₂O, 18 mL, 9 mmol, 2 equiv) dropwise at −78° C. under nitrogen atmosphere. The resulting mixture was stirred for 1 h at room temperature. The reaction was quenched with water (10 mL) at room temperature. The resulting mixture was extracted with ethyl acetate (EtOAc) (2×50 mL). The combined organic layers were washed with brine (1×20 mL), dried over anhydrous Na₂SO₄. After filtration, the filtrate was concentrated under reduced pressure. The reaction was repeated five times, and the batches combined for purification by reversed-phase flash chromatography (C18 silica gel; mobile phase, acetonitrile (MeCN) in water, 45% to 55% gradient in 10 min; detector, UV 254 nm) to provide an isomeric mixture (cis 4-Me/N-bridgehead, assumed) of tert-butyl 2-methyl-4-oxo-8-azabicyclo[3.2.1]octane-8-carboxylate (4.0 g, 63% yield). LCMS: (ES, m/z) RT=0.87 min, m/z=184.2 [M+H]⁺.

Step 3: A solution of an isomeric mixture of tert-butyl 2-methyl-4-oxo-8-azabicyclo[3.2.1]octane-8-carboxylate (trans Me-bridgehead, assumed) (1 g, 4.18 mmol, 1 equiv) and benzylamine (900 mg, 8.37 mmol, 2 equiv) in methanol (7 mL) was stirred for 16 h at 60° C. To the above mixture was added NaBH₃CN (790 mg, 12.5 mmol, 3 equiv) at 0° C. The resulting mixture was stirred for additional 1h at 60° C. The reaction was monitored by LCMS. The reaction was quenched with water (3 mL) at room temperature. The resulting mixture was extracted with EtOAc (2×20 mL). The combined organic layers were washed with brine (1×20 mL), dried over anhydrous Na₂SO₄. After filtration, the filtrate was concentrated under reduced pressure. The reaction was repeated for three times, and the batches combined for purification by silica gel column chromatography, eluting with dichloromethane/petroleum ether (1:1) to provide an isomeric mixture of tert-butyl 2-(benzylamino)-4-methyl-8-azabicyclo[3.2.1]octane-8-carboxylate (cis 4-Me/N-bridgehead, assumed) (3 g, 55% yield). LCMS: (ES, m/z) RT=0.71 min, m/z=331.2 [M+H]⁺.

Step 4: To a stirred solution of an isomeric mixture of tert-butyl (2-(benzylamino)-4-methyl-8-azabicyclo[3.2.1]octane-8-carboxylate (cis 4-Me/N-bridgehead, assumed) (3 g, 9.07 mmol, 1 equiv) and K₂CO₃ (3160 mg, 22.7 mmol, 2.5 equiv) in acetonitrile (30 mL) was added benzyl bromide (2330 mg, 13.61 mmol, 1.50 equiv) at room temperature. The resulting mixture was stirred for 5 h at 80° C. The reaction was monitored by LCMS. The reaction was quenched with water (10 mL) at room temperature. The resulting mixture was extracted with ethyl acetate (EtOAc) (2×50 mL). The combined organic layers were washed with brine (1×30 mL), dried over anhydrous Na₂SO₄. After filtration, the filtrate was concentrated under reduced pressure to provide a residue, which was purified by silica gel column chromatography, eluting with dichloromethane/petroleum ether (1:2) to provide an isomeric mixture of tert-butyl (2-(dibenzylamino)-4-methyl-8-azabicyclo[3.2.1]octane-8-carboxylate (cis 4-Me/N-bridgehead, assumed) (3.5 g, 92% yield). LCMS: (ES, m/z) RT=0.80 min, m/z=421.2 [M+H]⁺.

Step 5: Into a 100 mL round-bottom flask was added an isomeric mixture of tert-butyl (2-(dibenzylamino)-4-methyl-8-azabicyclo[3.2.1]octane-8-carboxylate (cis 4-Me/N-bridgehead, assumed) (3.5 g, 8.32 mmol, 1 equiv) and HCl(gas)(4M) in 1,4-dioxane (30 mL) at room temperature. The resulting mixture was stirred for 1 h at room temperature. The reaction was monitored by LCMS. The resulting mixture was concentrated under reduced pressure to provide crude isomeric mixture of N,N-dibenzyl-4-methyl-8-azabicyclo[3.2.1]octan-2-amine (cis 4-Me/N-bridgehead, assumed), which was used in next step directly without purification. LCMS: (ES, m/z) RT=0.52 min, m/z=321.0[M+H]⁺.

Step 6: Into a 100 mL round-bottom flask was added N,N-dibenzyl-4-methyl-8-azabicyclo[3.2.1]octan-2-amine (3 g, 9.36 mmol, 1 equiv), cyanogen bromide (1.98 g, 18.7 mmol, 2 equiv), K₂CO₃ (3.91 g, 28.1 mmol, 3 equiv), and acetonitrile (30 mL) at room temperature. The resulting mixture was stirred for 2 h at 80° C. The reaction was monitored by LCMS. The reaction was quenched with water (10 mL) at room temperature. The resulting mixture was extracted with ethyl acetate (EtOAc) (3×30 mL). The combined organic layers were washed with brine (1×30 mL), dried over anhydrous Na₂SO₄. After filtration, the filtrate was concentrated under reduced pressure to provide a crude isomeric mixture of 2-(dibenzylamino)-4-methyl-8-azabicyclo[3.2.1]octane-8-carbonitrile (cis 4-Me/N-bridgehead, assumed), which was used in the next step directly without purification. LCMS: (ES, m/z) RT=0.78 min, m/z=346.0[M+H]⁺.

Step 7: Into a 40 mL vial was added an isomeric mixture of 2-(dibenzylamino)-4-methyl-8-azabicyclo[3.2.1]octane-8-carbonitrile (cis 4-Me/N-bridgehead, assumed) (2.30 g, 6.65 mmol, 1 equiv), dibutyltin oxide (1.66 g, 6.65 mmol, 1 equiv), trimethylsilyl azide (2.30 g, 20.0 mmol, 3 equiv), and dimethylformamide (DMF) (20 mL) at room temperature. The resulting mixture was stirred for 2 h at 120° C. under nitrogen atmosphere. The reaction was monitored by LCMS. The residue was purified by reversed-phase flash chromatography (C18 silica gel; mobile phase, acetonitrile in water (0.1% trifluoroacetic acid), 30% to 50% gradient in 10 min; detector, UV 254 nm) to provide an isomeric mixture of N,N-dibenzyl-4-methyl-8-(1H-1,2,3,4-tetrazol-5-yl)-8-azabicyclo[3.2.1]octan-2-amine (cis 4-Me/N-bridgehead, assumed) (2.10 g, 72% yield). LCMS: (ES, m/z) RT=0.74 min, m/z=389.0[M+H]⁺.

Step 8: Into a 100 mL round-bottom flask was added an isomeric mixture of N,N-dibenzyl-4-methyl-8-(1H-1,2,3,4-tetrazol-5-yl)-8-azabicyclo[3.2.1]octan-2-amine (cis 4-Me/N-bridgehead, assumed) (2 g, 5.14 mmol, 1 equiv) in methanol (MeOH) (20 mL) was added Pd/C (1.36 g, 10.28 mmol, 2 equiv), and HCl (12M) (0.5 mL) at room temperature. The resulting mixture was stirred for 6 h at room temperature under hydrogen atmosphere. The reaction was monitored by LCMS. The resulting mixture was filtered, the filter cake was washed with MeOH (2×25 mL). The filtrate was concentrated under reduced pressure to provide an isomeric mixture of 4-methyl-8-(1H-1,2,3,4-tetrazol-5-yl)-8-azabicyclo[3.2.1]octan-2-amine, HCl salt (cis 4-Me/N-bridgehead, assumed) (1.20 g, 96% yield). LCMS: (ES, m/z) RT=0.25 min, m/z=209.0[M+H]⁺.

Step 9: A solution of 1-[4-(difluoromethoxy)phenyl]cyclopropane-1-carboxylic acid (“carboxyl (ii) reagent”) (500 mg, 2.19 mmol, 1 equiv) and 4-methyl-8-(1H-1,2,3,4-tetrazol-5-yl)-8-azabicyclo[3.2.1]octan-2-amine (“amine (i) reagent”) (456 mg, 2.19 mmol, 1 equiv), hydroxybenzotriazole (HOBT) (592.15 mg, 4.38 mmol, 2 equiv), 1-ethyl-3-(3-dimethylaninopropyl)carbodiimide (EDCI) (840 mg, 4.38 mmol, 2 equiv), diisopropylethyl amine (DIEA) (850 mg, 6.57 mmol, 3.00 equiv) in DMF (15 mL) was stirred for 2 h at room temperature. The reaction was monitored by LCMS. The reaction was quenched with water (5 mL) at room temperature.

The resulting mixture was extracted with EtOAc (3×15 mL). The combined organic layers were washed with water (2×5 mL) and dried over anhydrous Na₂SO₄. After filtration, the filtrate was concentrated under reduced pressure. The residue was purified by reversed-phase flash chromatography (C18 silica gel; mobile phase, acetonitrile in water, 35% to 45% gradient in 10 min; detector, UV 220 nm).

Step 10: The crude product (350 mg, 90% purity) was purified by PREP HPLC (Xselect CSH OBD Column 30*150 mm, 5 umn; Mobile Phase A: Water (0.05% TFA), Mobile Phase B: acetonitrile; Flow rate: 60 mL/min mL/min; Gradient: 40% B to 50% B in 10 min; Wave Length: 254 nm/220 nm) to provide an isomeric mixture (cis 4-Me/N-bridgehead, assumed) of Compound 35′* and Compound 35″*. The mixture of Compound 35′* and Compound 35″* (350 mg), was further purified by PREP HPLC (Xselect CSH OBD Column 30*150 mm, 5 umn; Mobile Phase A: Water(0.05% trifluoroacetic acid (TFA)), Mobile Phase B: acetonitrile; Flow rate: 60 mL/min mL/min; Gradient: 40% B to 50% B in 10 min; Wave Length: 254 nm/220 nm) to provide a first eluting isomeric mixture (cis at the 2 and 4 position, assumed, 170 mg) and a second eluting isomeric mixture (trans at the 2 and 4 position, assumed, 130 mg); stereochemistry arbitrarily assigned.

Step 11: The second eluting isomeric mixture (trans at the 2 and 4 position, assumed) was purified by chiral-PREP HPLC (CHIRALPAK ID, 2*25 cm, 5 μm; Mobile Phase A: hexanes (0.1% trifluoroacetic acid (TFA)), Mobile Phase B: ethanol; Flow rate: 20 mL/min; Gradient: 12% B to 12% B in 16 min; Wave Length: 220/254 nm) to provide Compound 35A* (RT(min): 13.09, 62.5 mg) and Compound 35D* (RT(min): 15.32, 58.9 mg); stereochemistry arbitrarily assigned.

Compound 35A*; LCMS (ES, m/z): RT=1.547 min, m/z=419.6[M+H]⁺; 1H NMR (400 MHz, Methanol-d4) δ 7.45 (d, J=8.8 Hz, 2H), 7.17 (d, J=8.8 Hz, 2H), 6.86 (t, J=74 Hz, 1H), 4.21 (s, 1H), 4.22-4.11 (m, 1H), 4.08 (d, J=7.4 Hz, 1H), 1.88 (t, J=7.1 Hz, 2H), 1.75-1.46 (m, 6H), 1.41-1.38 (m, 1H), 1.13-1.09 (m, 5H).

Compound 35D*; LCMS (ES, m/z): RT=1.547 min, m/z=419.25[M+H]⁺; 1H NMR (400 MHz, Methanol-d4) δ 7.47 (d, J=8.8 Hz, 2H), 7.19 (d, J=8.8 Hz, 2H), 7.22-7.14 (m, 1H), 6.86 (t, J=74 Hz, 1H), 4.21 (t, J=4.6 Hz, 1H), 4.22-4.11 (m, 1H), 4.11-4.04 (m, 1H), 2.02-1.84 (m, 2H), 1.74-1.45 (m, 6H), 1.42-1.35 (m, 1H), 1.13-1.11 (m, 5H).

Step 12: The first eluting isomeric mixture (cis at the 2 and 4 position, assumed) was purified by chiral-PREP HPLC (CHIRALPAK IC, 2*25 cm, 5 μm; Mobile Phase A: Hexanes (0.1% trifluoroacetic acid), Mobile Phase B: ethanol:dichloromethane=1:1; Flow rate: 20 mL/min; Gradient: 20% B to 20% B in 11.5 min; Wave Length: 220/254 nm) to provide Compound 35B* (RT(min): 7.19, 14.7 mg) and Compound 35C* (RT(min): 9.89, 15 mg); stereochemistry arbitrarily assigned.

Compound 35B*; LCMS (ES, m/z): RT=1.555 min, m/z=419.2[M+H]+ 1H NMR (400 MHz, Methanol-d4) δ 7.35 (dd, J=6.0 Hz, 2.0 Hz, 2H), 6.97 (d, J=8.4 Hz, 2H), 6.72 (t, 1H), 4.20 (d, J=5.1 Hz, 1H), 3.94 (d, J=4.8 Hz, 1H), 3.88 (d, J=4.8 Hz, 1H), 2.10 (dt, J=15.2, 6.1 Hz, 1H), 1.99-1.86 (m, 2H), 1.90-1.73 (m, 3H), 1.58 (ddd, J=9.5, 6.2, 2.9 Hz, 1H), 1.51 (ddd, J=9.5, 6.2, 3.1 Hz, 1H), 1.25 (dt, J=15.2, 1.4 Hz, 1H), 1.15 (ddd, J=9.6, 6.2, 3.1 Hz, 1H), 1.07 (ddd, J=9.1, 6.2, 3.0 Hz, 1H), 0.75 (d, J=7.3 Hz, 3H).

Compound 35C*; LCMS (ES, m/z): RT=1.572 min, m/z=419.2[M+H]+. 1H NMR (400 MHz, Methanol-d4) δ 7.35 (d, J=2.8 Hz, 2H), 6.97 (d, J=4.4 Hz, 2H), 6.71 (t, 1H), 4.20 (d, J=4.3 Hz, 1H), 3.96-3.90 (m, 1H), 3.90-3.84 (m, 1H), 2.10 (dt, J=15.2, 6.1 Hz, 1H), 2.00-1.89 (m, 2H), 1.93-1.73 (m, 3H), 1.58 (ddd, J=9.5, 6.2, 2.9 Hz, 1H), 1.51 (ddd, J=9.5, 6.2, 3.1 Hz, 1H), 1.25 (dt, J=15.3, 1.4 Hz, 1H), 1.15 (ddd, J=9.7, 6.2, 3.1 Hz, 1H), 1.07 (ddd, J=9.1, 6.2, 2.9 Hz, 1H), 0.7 (d, J=7.3 Hz, 3H).

### Part 2—Synthesis of Compounds 35′″*, 35″″*, and 35E*, 35F*, 35G* and 35H*

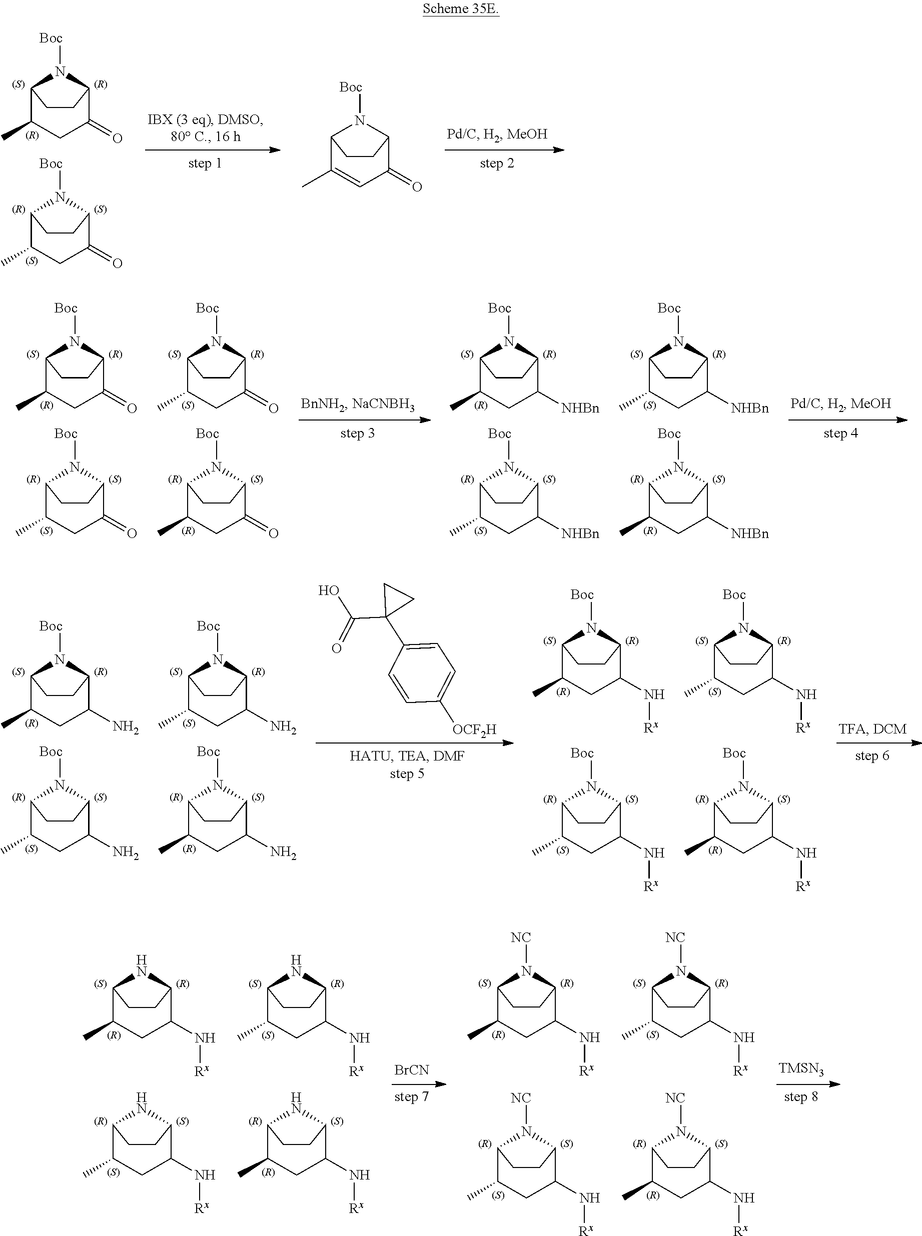

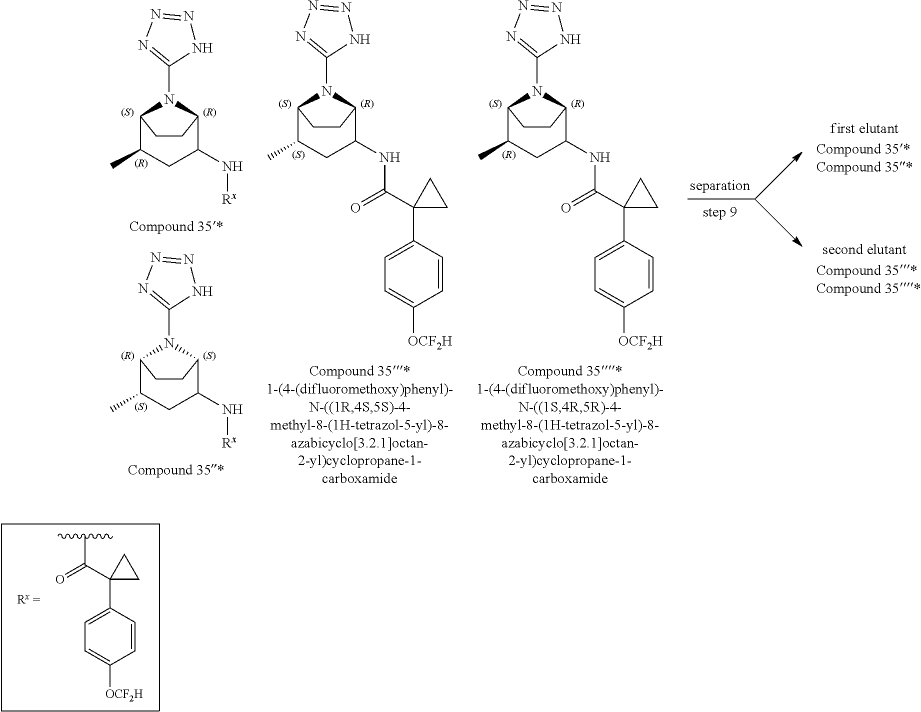

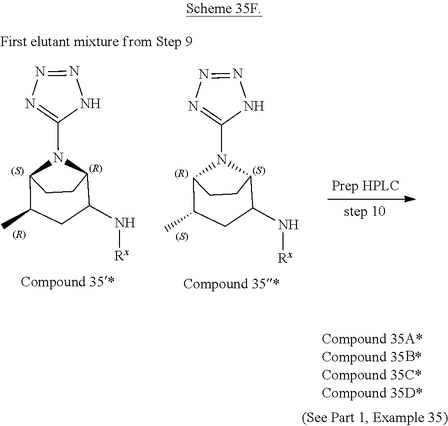

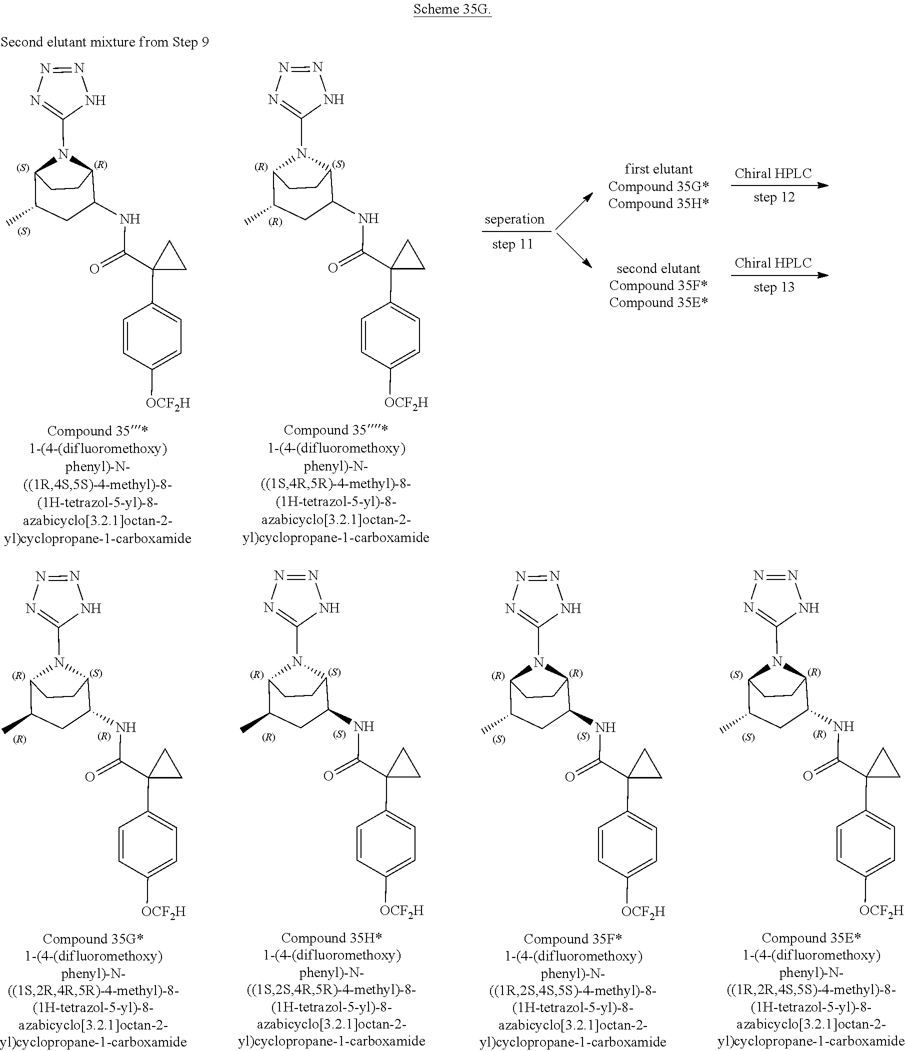

Step 1: Into a 40 mL vial was added the mixture of tert-butyl-2-methyl-4-oxo-8-azabicyclo[3.2.1]octane-8-carboxylate (prepared from Part 1, step 2, of Example 35) (1.00 g, 4.17 mmol, 1 equiv) and 2-iodoxybenzoic acid (IBX) (3.51 g, 12.5 mmol, 3 equiv), dimethylsulfoxide (DMSO) (6 mL) at room temperature. The resulting mixture was stirred overnight at 80° C. under air atmosphere. The reaction was monitored by LCMS. The residue was purified by reversed-phase flash chromatography (C18 silica gel; mobile phase, acetonitrile in water, 30% to 38% gradient in 20 min; detector, UV 220 nm) to provide tert-butyl 2-methyl-4-oxo-8-azabicyclo[3.2.1]oct-2-ene-8-carboxylate (600 mg, 61% yield). LCMS (ES, m/z): RT=0.709 min, m/z=182[M+H]+.

Step 2: To a solution of tert-butyl 2-methyl-4-oxo-8-azabicyclo[3.2.1]oct-2-ene-8-carboxylate (100 mg, 0.42 mmol, 1 equiv) in methanol (MeOH) (3 mL) was added 10% Pd/C (134.5 mg, 1.26 mmol, 3 equiv). The mixture was hydrogenated at room temperature for 2 h under hydrogen atmosphere. The resulting mixture was filtered, the filter cake was washed with MeOH (3×10 mL). The filtrate was concentrated under reduced pressure. The crude product, tert-butyl 2-methyl-4-oxo-8-azabicyclo[3.2.1]octane-8-carboxylate, comprising a mixture of 4 stereoisomers, was used in the next step directly without further purification. LCMS (ES, m/z): RT=0.817 min, m/z=182[M+H]+.

Step 3: Into a 40 mL vial were added tert-butyl 2-methyl-4-oxo-8-azabicyclo[3.2.1]octane-8-carboxylate (400 mg, 1.67 mmol, 1 equiv), benzylamine (215 mg, 2.00 mmol, 1.2 equiv), and methanol (MeOH) (10 mL) at room temperature. The resulting mixture was stirred overnight at 60° C. under nitrogen atmosphere was added NaBH₃CN (158 mg, 2.50 mmol, 1.5 equiv) in portions at room temperature under nitrogen atmosphere. The resulting mixture was stirred for 2 h at 60° C. under nitrogen atmosphere. The reaction was monitored by LCMS. The reaction was quenched with ice water (10 mL) at 0° C. The aqueous layer was extracted with ethyl acetate (EtOAc) (3×30 mL).The residue was purified by reversed-phase flash chromatography (C18 silica gel; mobile phase, acetonitrile in water, 30% to 50% gradient in 10 min; detector, UV 220 nm) to provide tert-butyl 2-(benzylamino)-4-methyl-8-azabicyclo[3.2.1]octane-8-carboxylate as a mixture of 8 stereoisomers (500 mg, 91% yield). LCMS (ES, m/z): RT=0.709 min, m/z=331[M+H]⁺.

Step 4: To a solution of tert-butyl 2-(benzylamino)-4-methyl-8-azabicyclo[3.2.1]octane-8-carboxylate (500 mg, 1.51 mmol, 1 equiv) in 20 mL methanol (MeOH) was added 10% Pd/C (500 mg, 4.70 mmol, 3.1 equiv). The mixture was hydrogenated at room temperature for 2 h under hydrogen atmosphere. The resulting mixture was filtered, the filter cake was washed with MeOH (3×10 mL). The filtrate was concentrated under reduced pressure to provide tert-butyl 2-amino-4-methyl-8-azabicyclo[3.2.1]octane-8-carboxylate (300 mg, 83% yield) as a mixture of 8 stereoisomers, which was used in the next step directly without further purification. LCMS (ES, m/z): RT=0.586 min, m/z=241[M+H]+.

Step 5: Into a 20 mL vial were added tert-butyl 2-amino-4-methyl-8-azabicyclo[3.2.1]octane-8-carboxylate (300 mg, 1.24 mmol, 1 equiv) and 1-[4-(difluoromethoxy)phenyl]cyclopropane-1-carboxylic acid (313 mg, 1.37 mmol, 1.1 equiv), (1-[bis(dimethylamnio)methylene]-1H-1,2,3-triazolo[4,5-b]pyridinium 3-oxide hexafluorophosphate (HATU) (569 mg, 1.49 mmol, 1.2 equiv), triethyl amine (TEA) (378.9 mg, 3.74 mmol, 3 equiv), and dimethyl formamide (DMF) (5 mL) at room temperature. The resulting mixture was stirred for 2 h at room temperature under nitrogen atmosphere. The reaction was monitored by LCMS. The reaction was quenched with water (10 mL) at room temperature. The aqueous layer was extracted with ethyl acetate (EtOAc) (3×50 mL), dried, and concentrated under reduced pressure to provide a residue, which was purified by reversed-phase flash chromatography (C18 silica gel; mobile phase, acetonitrile in water, 50% to 60% gradient in 10 min; detector, UV 210 nm) to provide tert-butyl 2-{1-[4-(difluoromethoxy)phenyl]cyclopropaneamido}-4-methyl-8-azabicyclo[3.2.1]octane-8-carboxylate (350 mg, 63% yield) as a mixture of 8 stereoisomers. LCMS (ES, m/z): RT=1.091 min, m/z=451.1[M+H]+.

Step 6: Into a 20 mL vial were added tert-butyl 2-{1-[4-(difluoromethoxy)phenyl]cyclopropaneamido}-4-methyl-8-azabicyclo[3.2.1]octane-8-carboxylate (350 mg, 0.77 mmol, 1 equiv), dichloromethane (DCM) (5 mL), and trifluoroacetic acid (TFA) (2 mL) at room temperature. The resulting mixture was stirred for 2 h at room temperature under air atmosphere. The resulting mixture was concentrated under reduced pressure and lyophilized to provide 1-[4-(difluoromethoxy)phenyl]-N-{4-methyl-8-azabicyclo[3.2.1]octan-2-yl}cyclopropane-1-carboxamide (280 mg) as a mixture of 8 stereoisomers, which was used in the next step directly without further purification. LCMS (ES, m/z): RT=0.644 min, m/z=351.2[M+H]+.

Step 7: Into a 40 mL vial was added 1-[4-(difluoromethoxy)phenyl]-N-{4-methyl-8-azabicyclo[3.2.1]octan-2-yl}cyclopropane-1-carboxamide (280 mg, 0.79 mmol, 1 equiv), acetonitrile (10 mL), K₂CO₃ (331 mg, 2.39 mmol, 3 equiv), and BrCN (169 mg, 1.59 mmol, 2 equiv) at room temperature. The resulting mixture was stirred for 2 h at room temperature under nitrogen atmosphere.

The reaction was monitored by LCMS. The reaction was quenched by the addition of water (10 mL) at room temperature. The resulting mixture was extracted with ethyl acetate (EtOAc) (3×50 mL). The combined organic layers were washed with water (3×10 mL), dried over anhydrous Na₂SO₄. After filtration, the filtrate was concentrated under reduced pressure to provide N-{8-cyano-4-methyl-8-azabicyclo[3.2.1]octan-2-yl}-1-[4-(difluoromethoxy)phenyl]cyclopropane-1-carboxamide (260 mg, 87% yield) as a mixture of 8 stereoisomers. LCMS (ES, m/z): RT=0.895 min, m/z=376.3[M+H]+.

Step 8-9: Into a 25 mL sealed tube were added N-{8-cyano-4-methyl-8-azabicyclo[3.2.1]octan-2-yl}-1-[4-(difluoromethoxy)phenyl]cyclopropane-1-carboxamide (260 mg, 0.69 mmol, 1 equiv), dimethyl formamide (DMF) (5 mL), NH₄Cl (111 mg, 2.07 mmol, 3 equiv), and azidotrimethylsilane (TMSN₃) (239 mg, 2.07 mmol, 3 equiv) at room temperature. The resulting mixture was stirred for 2 h at 110° C. under nitrogen atmosphere. The reaction was monitored by LCMS. The residue was purified by reversed-phase flash chromatography (C18 silica gel; mobile phase, acetonitrile in water, 25% to 40% gradient in 15 min; detector, UV 200 nm) to provide as the first elutant a mixture of Compounds 35′* and 35″* (LCMS (ES, m/z): RT=0.739 min, m/z=419.2[M+H]+, 110 mg, 38% yield) and as the second elutant a mixture of Compounds 35′″* and 35″″* (LCMS (ES, m/z): RT=0.815 min, m/z=419.2[M+H]+, 100 mg, 35% yield).

Step 10: The first elutant from step 9 (a mixture of Compounds 35′* and 35″*) (110 mg) was purified by PREP HPLC (XSelect CSH Prep C18 5 μm, 19*250 mm Column; Mobile Phase A: water (0.05% trifluoroacetic acid (TFA)), Mobile Phase B: acetonitrile; Flow rate: 20 mL/min mL/min; Gradient: 2% B to 2% B in 2 min, 35% B to 45% B in 10 min, 45% B to 45% B in 13 min; Wave Length: 254 nm/220 nm) to provide a mixture of Compounds 35A* and 35D* (54 mg) as first eluting peak and Compounds 35B* and 35C* (18.3 mg) as second eluting peak. Further isolation and characterization of Compounds 35A*, 35D*, 35C* and 35B* is provided in Example 35, Part 1.

Step 11: The second elutant from step 9 (a mixture of Compounds 35′″* and 35″″*) (100 mg) was further purified by PREP HPLC (XSelect CSH Prep Fluoro-Phenyl, 19*250 mm, 5 μm; Mobile Phase A: Water (0.05% trifluoroacetic acid (TFA)), Mobile Phase B: acetonitrile; Flow rate: 20 mL/min mL/min; Gradient: 2% B to 2% B in 2 min, 39% B to 49% B in 10 min, 49% B to 49% B in 13 min; Wave Length: 254 nm/220 nm) to provide an assumed mixture of Compounds 35G* and 35H* (RT(min): 11.3, 26 mg) as the first elutant, and an assumed mixture of Compounds 35F* and 35E* (47 mg) as the second elutant. LCMS (ES, m/z): RT=0.740 min, m/z=419.2[M+H]+.

Step 12: The first elutant from step 11 (an assumed mixture of Compounds 35G* and 35H*) (26 mg) was purified by PREP-CHIRAL HPLC (CHIRAL ART Cellulose-SC, 2*25 cm, 5 μm; Mobile Phase A: hexanes (0.5% 2M NH₃-MeOH), Mobile Phase B: ethanol:dichloromethane=1:1; Flow rate: 20 mL/min; Gradient: 30% B to 30% B in 21 min; Wave Length: 220/254 nm) to provide Compound 35G* (RT(min): 7.75, 7.2 mg) and Compound 35H* (RT(min): 11.54, 4.9 mg). Stereochemistry arbitrarily assigned.

Compound 35G*: LCMS (ES, m/z): RT=0.694 min, m/z=419.2[M+H]+; ¹H NMR (400 MHz, Methanol-d4) δ 7.33-7.27 (m, 2H), 7.02-6.95 (m, 2H), 6.66 (d, J=74.0 Hz, 1H), 4.20 (dd, J=7.3, 3.0 Hz, 1H), 3.90-3.84 (m, 2H), 2.08-1.81 (m, 2H), 1.77-1.69 (m, 2H), 1.73-1.63 (m, 1H), 1.59-1.41 (m, 4H), 1.31 (d, J=4.0 Hz, 1H), 1.15-1.02 (m, 2H), 0.88 (d, J=6.8 Hz, 3H).

Compound 35H*: LCMS (ES, m/z): RT=0.698 min, m/z=419.2[M+H]+; ¹H NMR (400 MHz, Methanol-d4) δ 7.34-7.26 (m, 2H), 7.02-6.95 (m, 2H), 6.75 (t, J=74.0 Hz, 1H), 4.23-4.16 (m, 1H), 3.87 (d, J=2.8 Hz, 2H), 2.03-1.81 (m, 2H), 1.71 (td, J=11.5, 10.6, 5.1 Hz, 3H), 1.51 (ddq, J=16.3, 14.5, 5.1, 4.6 Hz, 4H), 1.15-1.02 (m, 2H), 0.88 (d, J=6.8 Hz, 3H).

Step 13: The first second from step 11 (an assumed mixture of Compounds 35F* and 35E*) (47 mg) was purified further by PREP-CHIRAL HPLC (CHIRAL ART Cellulose-SC, 2*25 cm, 5 μm; Mobile Phase A: hexanes (0.5% 2M NH₃-MeOH), Mobile Phase B: ethanol:dichloromethane=1:1; Flow rate: 20 mL/min; Gradient: 30% B to 30% B in 27 min; Wave Length: 220/254 nm) to provide Compound 35F* (RT(min): 11.38, 10.0 mg) and Compound 35E* (RT(min): 16.58, 9.4 mg). Stereochemistry arbitrarily assigned.

Compound 104F*: LCMS (ES, m/z): RT=0.769 min, m/z=419.2[M+H]+. ¹H NMR (400 MHz, Methanol-d4) δ 7.50-7.42 (m, 2H), 7.23-7.14 (m, 2H), 6.87 (t, J=74.0 Hz, 1H), 4.12 (d, J=5.4 Hz, 1H), 3.96 (ddd, J=11.9, 5.4, 3.3 Hz, 2H), 2.03 (dddd, J=11.8, 7.3, 4.8, 2.7 Hz, 1H), 1.85 (ddt, J=11.4, 8.2, 4.6 Hz, 2H), 1.81-1.74 (m, 1H), 1.70-1.45 (m, 4H), 1.13 (dq, J=4.8, 2.7 Hz, 2H), 1.13-1.02 (m, 1H), 0.89 (d, J=6.8 Hz, 3H).

Compound 104E*: LCMS (ES, m/z): RT=0.913 min, m/z=419.15[M+H]+. ¹H NMR (400 MHz, Methanol-d4) δ 7.50-7.42 (m, 2H), 7.23-7.14 (m, 2H), 6.87 (t, J=74.0 Hz, 1H), 4.12 (t, J=4.4 Hz, 1H), 3.96 (ddd, J=12.0, 5.6, 3.3 Hz, 2H), 2.03 (dddd, J=11.7, 7.3, 4.8, 2.7 Hz, 1H), 1.93-1.73 (m, 3H), 1.70-1.53 (m, 2H), 1.57-1.45 (m, 2H), 1.18-1.08 (m, 3H), 0.89 (d, J=6.8 Hz, 3H).

### Example 36. 1-(4-chlorophenyl)-N-(6-methyl-1-(1H-tetrazol-5-yl)azepan-3-yl)cyclopropane-1-carboxamide (rac-36), and Compounds 36A*, 36B*, 36E*, and 36D*

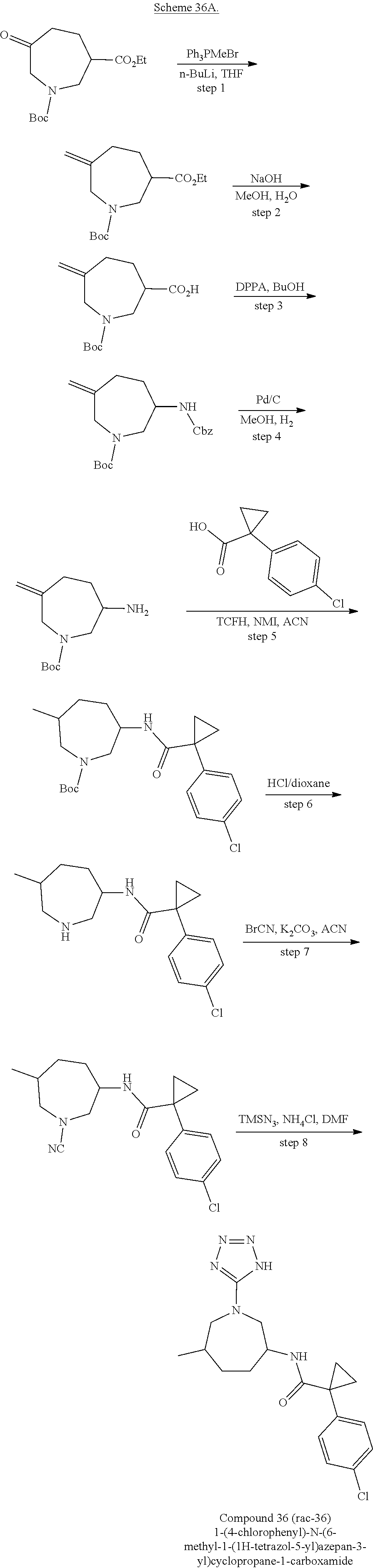

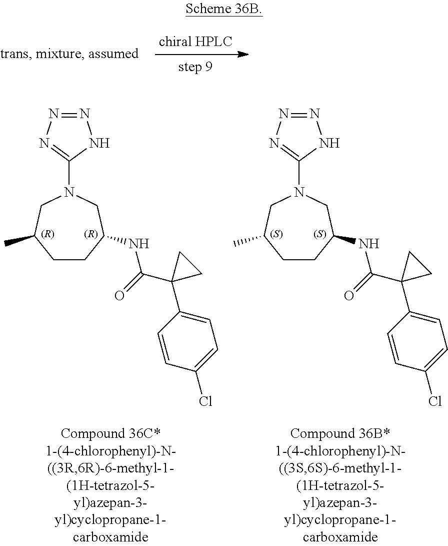

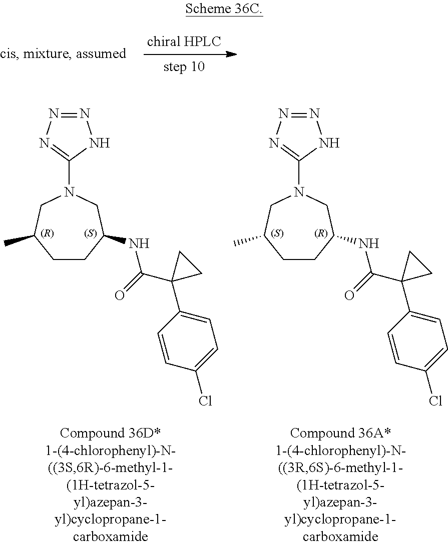

### Example 36 Follows Protocol C

Step 1: To a solution of bromo(methyl)triphenyl-lambda5-phosphane (1.79 g, 5.01 mmol, 1.10 equiv) in tetrahydrofuran (THF) (26 mL) was added n-butyl lithium (n-BuLi) (2.5M in THF, 2 mL, 5.01 mmol, 1.10 equiv) at 0° C., and the reaction was stirred for 30 min at 0° C. under nitrogen atmosphere. To the above mixture was added 1-tert-butyl 3-ethyl 6-oxoazepane-1,3-dicarboxylate (1.30 g, 4.66 mmol, 1 equiv). The resulting mixture was stirred for an additional 3 h at 0° C., and the reaction progress was monitored by LCMS. The reaction was quenched with saturated NH₄Cl (50 ml) at 0° C. The resulting mixture was extracted with ethyl acetate (EtOAc) (3×200 mL) and dried over anhydrous Na₂SO₄. After filtration, the filtrate was concentrated under reduced pressure. The residue was purified by reverse flash chromatography (C18 silica gel; mobile phase, acetonitrile in water, 0% to 100% gradient in 30 min; detector, UV 220/254 nm) to provide 1-tert-butyl 3-ethyl 6-methylideneazepane-1,3-dicarboxylate (600 mg, 47% yield). LCMS (ES, m/z): RT=0.967 min, m/z=284[M+1]⁺.

Step 2: Into a 20 mL vial was added 1-tert-butyl 3-ethyl 6-methylideneazepane-1,3-dicarboxylate (500 mg, 1.76 mmol, 1 equiv), sodium hydroxide (141 mg, 3.52 mmol, 2 equiv), methanol (MeOH) (10 mL), and H₂O (1 mL) at room temperature. The resulting mixture was stirred for 1 h at room temperature under nitrogen atmosphere, and the reaction progress was monitored by LCMS. The reaction was quenched with 30 ml of water, and the resulting mixture was extracted with ethyl acetate (EtOAc) (3×50 mL). The combined water layers were adjusted to pH 5 with HCl (1M), and the resulting mixture was further extracted with EtOAc (3×50 mL), dried over anhydrous Na₂SO₄, filtered, and the filtrate concentrated under reduced pressure to provide 1-(tert-butoxycarbonyl)-6-methylideneazepane-3-carboxylic acid (420 mg, 78% yield). LCMS (ES, m/z): RT=0.430 min, m/z=256[M+1]⁺.

Step 3: Into a 20 mL vial was added 1-(tert-butoxycarbonyl)-6-methylideneazepane-3-carboxylic acid (400 mg, 1.56 mmol, 1 equiv), benzyl alcohol (0.20 mL, 1.84 mmol, 4.7 equiv), triethylamine (793 mg, 7.83 mmol, 5 equiv), and toluene (8 mL) at room temperature. The resulting mixture was stirred for 2 h at 100° C. under nitrogen atmosphere, and the reaction progress was monitored by LCMS. The reaction was quenched with 20 mL of water, and resulting mixture was extracted with ethyl acetate (EtOAc) (3×30 mL), dried over anhydrous Na₂SO₄, filtered, and after filtration, the filtrate was concentrated under reduced pressure to provide a residue which was purified by reverse flash chromatography (C18 silica gel; mobile phase, acetonitrile in water, 0% to 100% gradient in 30 min; detector, UV 220/254 nm) to provide tert-butyl 3-{[(benzyloxy)carbonyl]amino}-6-methylideneazepane-1-carboxylate (200 mg, 35% yield). LCMS (ES, m/z): RT=1.07 min, m/z=361[M+1]⁺.

Step 4: Into a 100 mL round-bottom flask was added tert-butyl 3-{[(benzyloxy)carbonyl]amino}-6-methylideneazepane-1-carboxylate (200 mg, 0.55 mmol, 1 equiv) and Pd/C (50 mg, 0.47 mmol, 0.85 equiv), and methanol (MeOH) (25 mL) at room temperature. The resulting mixture was stirred for 1 h at room temperature under hydrogen atmosphere, and the reaction progress was monitored by LCMS. The resulting mixture was filtered, the filter cake was washed with MeOH (3×5 mL), and the filtrate was concentrated under reduced pressure to provide tert-butyl 3-amino-6-methylazepane-1-carboxylate (120 mg, 57% yield). LCMS (ES, m/z): RT=0.94 min, m/z=229[M+1]⁺.

Step 5: Into a 20 mL vial was added tert-butyl 3-amino-6-methylazepane-1-carboxylate (“amine (iv) reagent”) (150 mg, 0.65 mmol, 1 equiv), 1-(4-chlorophenyl)cyclopropane-1-carboxylic acid (“carboxyl (ii) reagent”) (155 mg, 0.78 mmol, 1.20 equiv), N,N,N′,N′-tetramethylchloroformamidinium hexafluorophosphate (TCFH) (240 mg, 0.85 mmol, 1.30 equiv), N-methyl imidazole (NMI) (270 mg, 3.28 mmol, 5.00 equiv), and acetonitrile (7.5 mL) at room temperature. The resulting mixture was stirred for 1 h at room temperature, and the reaction progress was monitored by LCMS. The reaction was quenched with 10 mL of water, extracted with ethyl acetate (3×30 mL), dried over anhydrous Na₂SO₄, filtered, the filtrate concentrated under reduced pressure to provide a residue, which was purified by reverse flash chromatography (C18 silica gel; mobile phase, acetonitrile in water, 0% to 100% gradient in 30 min; detector, UV 220/254 nm) to provide tert-butyl 3-[1-(4-chlorophenyl)cyclopropaneamido]-6-methylazepane-1-carboxylate (130 mg, 49% yield).

Step 6: Into a 20 mL vial were added tert-butyl 3-[1-(4-chlorophenyl)cyclopropaneamido]-6-methylazepane-1-carboxylate (230 mg, 0.56 mmol, 1 equiv) and HCl(gas) in 1,4-dioxane (4M, 10.00 mL) at room temperature for one hour, and the reaction progress was monitored by LCMS. After the reaction was completed, the resulting mixture was concentrated under vacuum to provide 1-(4-chlorophenyl)-N-(6-methylazepan-3-yl)cyclopropane-1-carboxamide as an HCl salt (130 mg). LCMS (ES, m/z): RT=0.69 min, m/z=307[M+1]⁺.

Step 7: Into a 20 mL vial was added 1-(4-chlorophenyl)-N-(6-methylazepan-3-yl)cyclopropane-1-carboxamide HCl salt (110 mg, 0.32 mmol, 1 equiv) and BrCN (68.8 mg, 0.64 mmol, 2 equiv), potassium carbonate (44.4 mg, 0.64 mmol, 2 equiv), and acetonitrile (5 mL) at room temperature. The resulting mixture was stirred for 1 h at room temperature, the reaction progress was monitored by LCMS. The reaction was quenched with 10 mL of water, extracted with ethyl acetate (3×50 mL), dried over anhydrous Na₂SO₄, filtered, the filtrate concentrated under reduced pressure to provide a residue, which was purified by reverse flash chromatography (C18 silica gel; mobile phase, acetonitrile in water, 0% to 100% gradient in 40 min; detector, UV 220/254 nm) to provide 1-(4-chlorophenyl)-N-(1-cyano-6-methylazepan-3-yl)cyclopropane-1-carboxamide (70 mg, 59% yield).

Step 8: Into an 8 mL vial was added 1-(4-chlorophenyl)-N-(1-cyano-6-methylazepan-3-yl)cyclopropane-1-carboxamide (70 mg, 0.211 mmol, 1 equiv), trimethylsilyl azide (TMSN₃) (48.6 mg, 0.42 mmol, 2 equiv), dimethyl formamide (DMF) (2 mL), and NH₄Cl (45.1 mg, 0.844 mmol, 4 equiv) at room temperature. The resulting mixture was stirred for 2 h at 120° C., and the reaction progress was monitored by LCMS. The reaction was quenched with 10 mL of water, extracted with ethyl acetate (3×30 mL), dried over anhydrous Na₂SO₄, filtered, and the filtrate concentrated under reduced pressure to provide 1-(4-chlorophenyl)-N-[6-methyl-1-(1H-1,2,3,4-tetrazol-5-yl)azepan-3-yl]cyclopropane-1-carboxamide (Compound 36, rac-36), which was purified by reverse flash chromatography (C18 silica gel; mobile phase, acetonitrile in water, 0% to 100% gradient in 40 min; detector, UV 220/254 nm) to provide an assumed trans mixture (10 mg) as a first eluting peak and an assumed cis mixture (20 mg) as a second eluting peak. Stereochemistry arbitrarily assigned.

Step 9: The assumed trans product (10 mg) was purified by Chiral-PREP HPLC (Lux 5 μm Cellulose-4, 2.12*25 cm, 5 m, Mobile Phase A: hexanes (0.2% formic acid), Mobile Phase B: ethanol; Flow rate: 20 mL/min; Gradient: 20% B to 20% B in 20 min; Wave Length: 220 nm) to provide Compound 36B* (RT(min): 16.69, 2.2 mg) and Compound 36C* (RT(min): 19.17, 2.40 mg). Stereochemistry arbitrarily assigned.

Compound 36B*: LCMS (ES, m/z): RT=1.459 min, m/z=375[M+1]⁺. ¹H NMR (400 MHz, Methanol-d₄) δ 7.27 (s, 4H), 4.21-4.18 (m, 1H), 3.74-3.65 (m, 1H), 3.60-3.51 (m, 2H), 2.78-2.72 (m, 1H), 2.05 (d, J=10.4 Hz, 1H), 1.77-1.71 (m, 1H), 1.63-1.46 (m, 4H), 1.36-1.19 (m, 1H), 1.07-1.01 (m, 2H), 0.97 (d, J=6.7 Hz, 3H).

Compound 36C*: LCMS (ES, m/z): RT=1.460 min, m/z=375[M+1]⁺. ¹H NMR (400 MHz, Methanol-d₄) δ 7.27 (s, 4H), 4.21-4.18 (m, 1H), 3.70-3.51 (m, 3H), 2.78-2.74 (m, 1H), 2.05 (d, J=9.3 Hz, 1H), 1.77-1.71 (m, 1H), 1.64-1.47 (m, 4H), 1.25-1.21 (m, 1H), 1.08-1.02 (m, 2H), 0.97 (d, J=6.8 Hz, 3H).

Step 10: The assumed cis product (20 mg) was purified by Prep chiral HPLC (Lux 5 μm Cellulose-4, 2.12*25 cm, 5 m, Mobile Phase A: hexanes (0.2% formic acid), Mobile Phase B: ethanol; Flow rate: 20 mL/min; Gradient: 20% B to 20% B in 15 min; Wave Length: 220 nm) to provide Compound 36D* (RT(min): 11.44, 7.2 mg) and Compound 36A* (RT(min): 13.62, 8.0 mg). Stereochemistry arbitrarily assigned.

Compound 36A*: LCMS (ES, m/z): RT=1.596 min, m/z=375[M+1]⁺. ¹H NMR (400 MHz, Methanol-d₄) δ 7.39 (s, 4H), 4.03-4.00 (m, 1H), 3.71-3.51 (m, 3H), 2.95-2.89 (m, 1H), 1.87-1.82 (m, 2H), 1.76-1.64 (m, 1H), 1.60-1.48 (m, 2H), 1.29-1.18 (m, 2H), 1.16-1.06 (m, 2H), 0.96 (d, J=6.7 Hz, 3H).

Compound 36D*: LCMS (ES, m/z): RT=1.593 min, m/z=375[M+1]⁺. ¹H NMR (400 MHz, Methanol-d₄) δ 7.39 (s, 4H), 4.03-4.00 (m, 1H), 3.73-3.53 (m, 3H), 2.95-2.90 (m, 1H), 1.88-1.82 (m, 2H), 1.78-1.63 (m, 1H), 1.60-1.51 (m, 2H), 1.33-1.19 (m, 2H), 1.11-1.07 (m, 2H), 0.96 (d, J=6.7 Hz, 3H).

### Example 37. 1-(4-(difluoromethoxy)-2-fluorophenyl)-N-(4-methyl-8-(1H-tetrazol-5-yl)-8-azabicyclo[3.2.1]octan-2-yl)cyclopropane-1-carboxamide (rac-101), and Compounds 101A, 101B*, 101C*, and 101D*

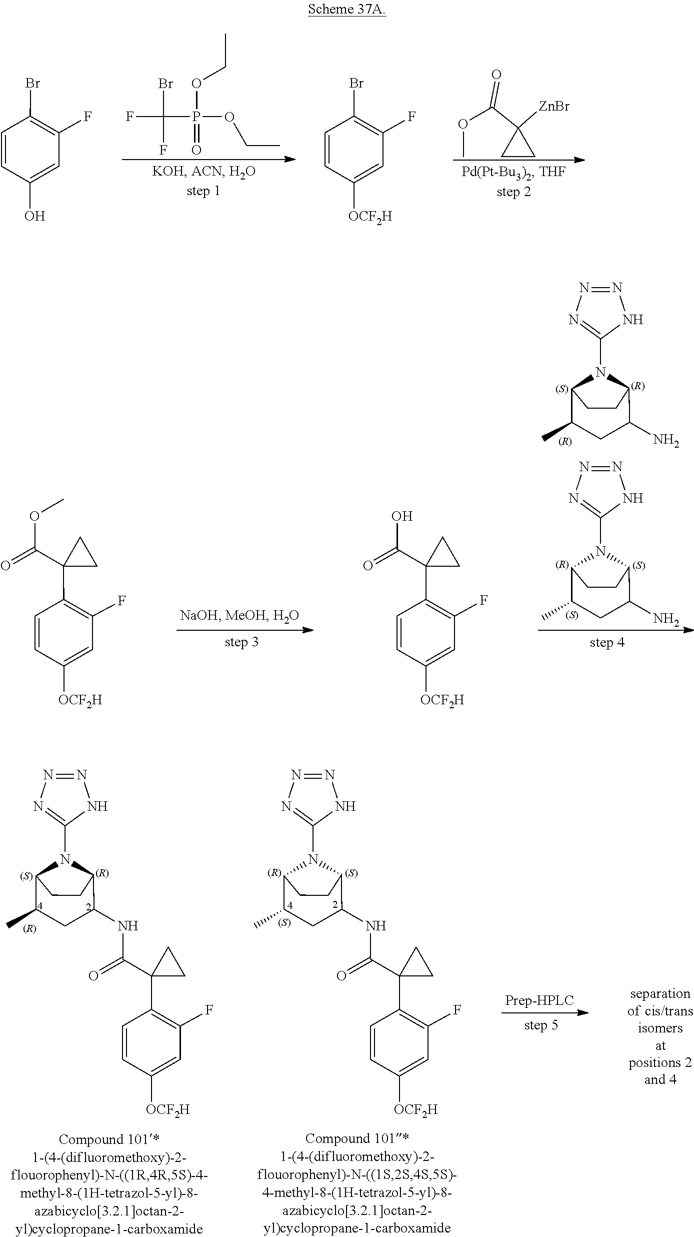

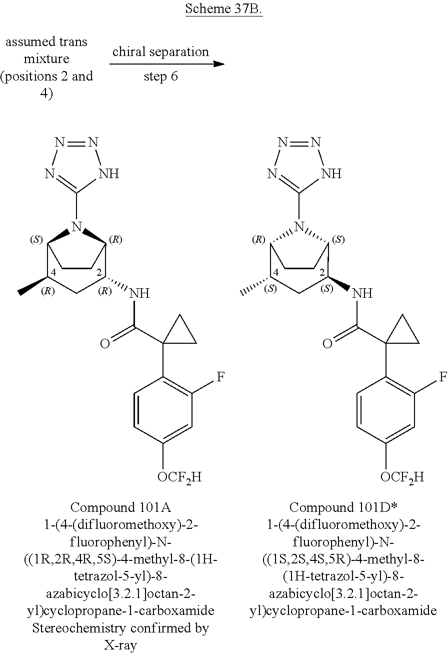

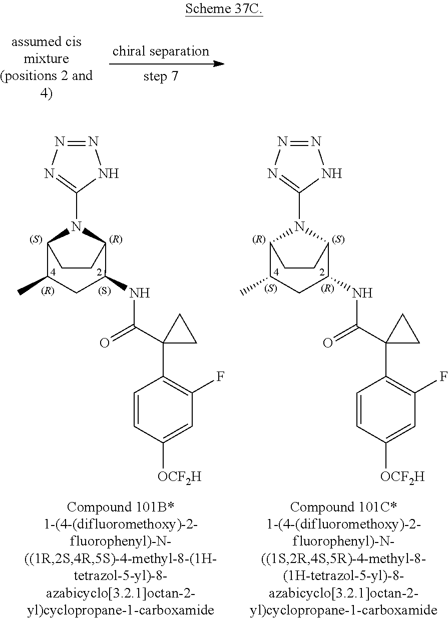

### Example 37 Follows Protocol A

Step 1: Into a 250 mL round-bottom flask were added 4-bromo-3-fluorophenol (5 g, 26.2 mmol, 1 equiv), acetonitrile (25 mL), H₂O (25 mL), and KOH (29.4 g, 523 mmol, 20.0 equiv). To the above solution was added diethyl bromodifluoromethylphosphonate (14 g, 52.4 mmol, 2 equiv) at 0° C. The resulting mixture was stirred for 2 h at room temperature under nitrogen atmosphere. The reaction was monitored by LCMS. The reaction was quenched with water (100 mL) at room temperature. The resulting mixture was extracted with ethyl acetate (EtOAc) (2×200 mL). The combined organic layers were washed with water (200 mL), dried over anhydrous Na₂SO₄, filtered, and the filtrate was concentrated under reduced pressure. The residue was purified by silica gel column chromatography, eluted with CH₂Cl₂/petroleum ether (1:5) to afford 1-bromo-4-(difluoromethoxy)-2-fluorobenzene (4 g, 63% yield). LCMS:(ES, m/z):RT=0.892 min, m/z=240.9 [M+H]⁺.

Step 2: Into a 250 mL round-bottom flask was added 1-bromo-4-(difluoromethoxy)-2-fluorobenzene (2 g, 8.29 mmol, 1 equiv), tetrahydrofuran (THF) (4 mL) and bis(tri-tert-butylphosphane) palladium (1.27 g, 2.49 mmol, 0.3 equiv) at room temperature. To the above solution was added bromo[1-(methoxycarbonyl)cyclopropyl]zinc (0.9M in THF, 46.1 mL, 41.5 mmol, 5 equiv) dropwise at 0° C. The resulting mixture was stirred for 2 h at 60° C. under nitrogen atmosphere. The reaction was monitored by LCMS. The reaction was quenched with water (100 mL) at room temperature. The resulting mixture was extracted with EtOAc (3×100 mL). The combined organic layers were washed with water (200 mL), dried over anhydrous Na₂SO₄, filtered, and the filtrate was concentrated under reduced pressure. The residue was purified by silica gel column chromatography, eluted with petroleum ether/ethyl acetate (1:1) to afford methyl 1-[4-(difluoromethoxy)-2-fluorophenyl]cyclopropane-1-carboxylate (1.8 g, 83% yield). LCMS:(ES, m/z):RT=0.846 min, m/z=261.1 [M+H]⁺.

Step 3: Into a 40 mL vial was added methyl 1-[4-(difluoromethoxy)-2-fluorophenyl]cyclopropane-1-carboxylate (1.8 g, 6.91 mmol, 1 equiv), methanol (MeOH) (18 mL), H₂O (6 mL), and NaOH (1.38 g, 34.6 mmol, 5 equiv) at room temperature. The resulting mixture was stirred for 16 h at 40° C. The reaction was monitored by LCMS. Upon completion, the mixture was adjusted to pH=5 with HCl(2M), and extracted with ethyl acetate (EtOAc) (3×100 mL). The combined organic layers were washed with water (150 mL), dried over anhydrous Na₂SO₄, filtered, and the filtrate was concentrated under reduced pressure. The residue was purified by silica gel column chromatography, eluted with petroleum ether/ethyl acetate (1:1) to afford 1-[4-(difluoromethoxy)-2-fluorophenyl]cyclopropane-1-carboxylic acid (1.50 g, 88% yield). LCMS:(ES, m/z):RT=0.717 min, m/z=247.1 [M+H]⁺.

Step 4: Into a 20 mL vial were added 1-[4-(difluoromethoxy)-2-fluorophenyl]cyclopropane-1-carboxylic acid (carboxyl (ii) reagent) (1 g, 4.06 mmol, 1 equiv) and an isomeric mixture of 4-methyl-8-(1H-1,2,3,4-tetrazol-5-yl)-8-azabicyclo[3.2.1]octan-2-amine, HCl salt (cis 4-Me/N-bridgehead, assumed) (from Example 35, step 8) (994 mg, 4.06 mmol, 1 equiv) (amine (i) reagent), hydroxybenzotriazole (HOBT) (1.09 g, 8.12 mmol, 2.00 equiv), 1-ethyl-3-(3-dimethylaminopropyl)carbodiimide (EDCI) (1.56 g, 8.12 mmol, 2 equiv), dimethyl formamide (DMF) (10 mL), and diisopropylethyl amine (DIEA) (1.57 g, 12.2 mmol, 3 equiv) at room temperature. The resulting mixture was stirred for 1 h at room temperature. The reaction was monitored by LCMS. The reaction was quenched with water(50 mL) at room temperature. The resulting mixture was extracted with ethyl acetate (EtOAc) (3×100 mL). The combined organic layers were washed with brine (200 mL), dried over anhydrous Na₂SO₄. After filtration, the filtrate was concentrated under reduced pressure to provide a mixture of Compound 101′* and Compound 101″*.

Step 5: The residue, a mixture of Compound 101′* and Compound 101″*, was purified by reverse-phase flash chromatography (C18 silica gel; mobile phase, acetonitrile in water, 0% to 100% gradient in 40 min; detector, UV 220/254 nm) to provide a first eluting isomeric mixture (assumed cis at the 2 and 4 position, 500 mg, 80% purity) and a second eluting isomeric mixture (assumed trans at the 2 and 4 position, 300 mg, 82% purity); stereochemistry arbitrarily assigned. The first eluting isomeric mixture was further purified by Prep-HPLC (XBridge Prep OBD C18 Column, 30*150 mm, 5 μm; Mobile Phase A: Water (10 mmol/L NH₄HCO₃), Mobile Phase B: acetonitrile; Flow rate: 60 mL/min; Gradient: 13% B to 23% B in 7.8 min; Wave Length: 254/220 nm; RT(min): 11.03) to afford a purified first eluting isomeric mixture (250 mg, 18% yield; LCMS:(ES, m/z):RT=0.716 min, m/z=437.1 [M+H]⁺). The second eluting isomeric mixture was further purified by Prep-HPLC (XBridge Prep OBD C18 Column, 30*150 mm, 5 μm; Mobile Phase A: Water (10 mmol/L NH₄HCO₃), Mobile Phase B: acetonitrile; Flow rate: 60 mL/min; Gradient: 19% B to 29% B in 7.8 min; Wave Length: 254/220 nm; RT(min): 11.87) to afford a purified second eluting isomeric mixture (140 mg, 10% yield; LCMS:(ES, m/z):RT=0.779 min, m/z=437.1 [M+H]⁺).

Step 6: The second eluting isomeric mixture from step 5 (180 mg, combination of two batches) was purified by Prep-Chiral-HPLC (CHIRALPAK SC 2*25 cm, 5 μm; Mobile Phase A: Hexanes (0.1% trifluoroacetic acid), Mobile Phase B: (ethanol:dichloromethane=1:1); Flow rate: 20 mL/min; Gradient: isocratic 30; Wave Length: 220/254 nm) to afford Compound 101A (65.3 mg, RT(min): 7.773) and Compound 101D* (66.2 mg, RT(min): 9.548). Absolute stereochemistry of Compound 101A confirmed by X-ray crystallography.

Compound 101A: LCMS:(ES, m/z):RT=1.588 min, m/z=437.1 [M+H]⁺. ¹H NMR (400 MHz, Methanol-d₄) δ 7.46 (t, J=8.4 Hz, 1H), 7.13-6.70 (m, 3H), 4.25-4.11 (m, 2H), 4.12-4.05 (m, 1H), 2.02-1.82 (m, 2H), 1.76-1.64 (m, 4H), 1.63-1.52 (m, 2H), 1.46-1.32 (m, 1H), 1.22-1.13 (m, 1H), 1.11 (d, J=7.2 Hz, 4H).

Compound 101D*: LCMS:(ES, m/z):RT=0.861 min, m/z=437.1 [M+H]⁺. ¹H NMR (400 MHz, Methanol-d₄) δ 7.46 (t, J=8.4 Hz, 1H), 7.15-6.70 (m, 3H), 4.25-4.13 (m, 2H), 4.13-4.04 (m, 1H), 2.05-1.83 (m, 2H), 1.78-1.64 (m, 4H), 1.64-1.51 (m, 2H), 1.44-1.35 (m, 1H), 1.22-1.13 (m, 1H), 1.11 (d, J=7.2 Hz, 4H).

Step 7: The first eluting isomeric mixture from step 5 (30 mg) was purified by Chiral-Prep-HPLC (CHIRALPAK ID, 2*25 cm, 5 μm; Mobile Phase A: Hexanes (0.1% trifluoroacetic acid), Mobile Phase B: ethanol:dichloromethane=1:1; Flow rate: 20 mL/min; Gradient: 30% B to 30% B in 7 min; Wave Length: 220/254 nm) to afford Compound 101B* (13.5 mg, RT(min): 4.44) and Compound 101C* (13.5 mg, RT(min): 5.94). Stereochemistry was arbitrarily assigned.

Compound 101B*: LCMS:(ES, m/z):RT=1.460 min, m/z=437.1 [M+H]⁺. ¹H NMR (400 MHz, Methanol-d₄) δ 7.37 (t, J=8.4 Hz, 1H), 7.04-6.55 (m, 3H), 4.20 (d, J=3.8 Hz, 1H), 3.96 (d, J=5.2 Hz, 1H), 3.94-3.84 (m, 1H), 2.18-2.05 (m, 1H), 2.05-1.89 (m, 2H), 1.86-1.73 (m, 3H), 1.63-1.51 (m, 2H), 1.35-1.25 (m, 1H), 1.17-1.05 (m, 2H), 0.85-0.78 (m, 3H).

Compound 101C*: LCMS:(ES, m/z):RT=1.454 min, m/z=437.1 [M+H]⁺. ¹H NMR (400 MHz, Methanol-d₄) δ 7.37 (t, J=8.4 Hz, 1H), 7.04-6.59 (m, 3H), 4.20 (d, J=4.4 Hz, 1H), 3.96 (d, J=4.8 Hz, 1H), 3.92-3.83 (m, 1H), 2.16-2.01 (m, 1H), 2.01-1.91 (m, 2H), 1.86-1.76 (m, 3H), 1.64-1.53 (m, 2H), 1.35-1.25 (m, 1H), 1.16-1.06 (m, 2H), 0.85-0.77 (m, 3H).

### Example 38. 1-(2,6-difluoro-4-methoxyphenyl)-N-((1R,2R,4R,5S)-4-methyl-8-(1H-tetrazol-5-yl)-8-azabicyclo[3.2.1]octan-2-yl)cyclopropane-1-carboxamide (Compound 102A*) and 1-(2,6-difluoro-4-methoxyphenyl)-N-((1S,2S,4S,5R)-4-methyl-8-(1H-tetrazol-5-yl)-8-azabicyclo[3.2.1]octan-2-yl)cyclopropane-1-carboxamide (Compound 102D*)

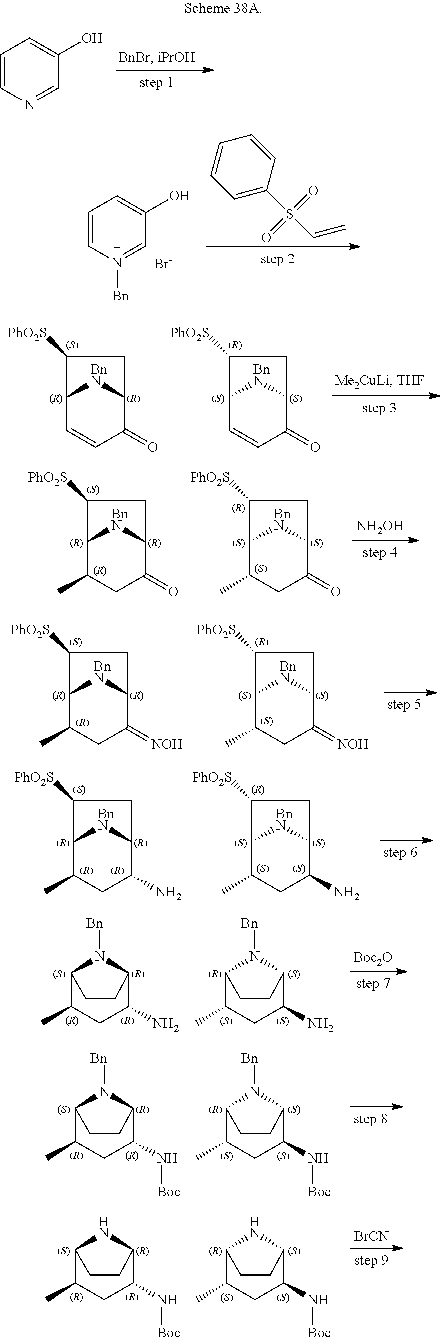

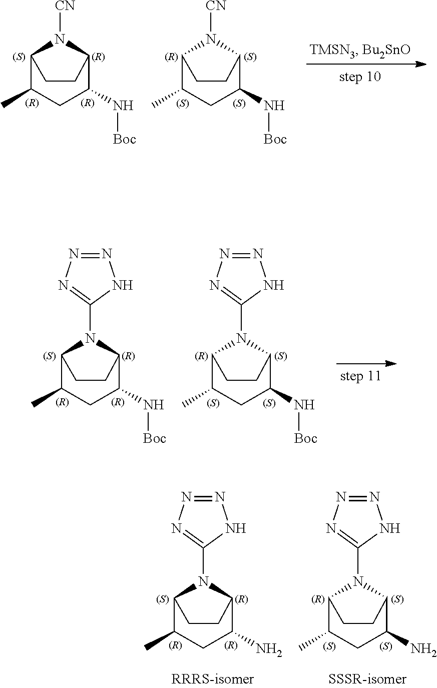

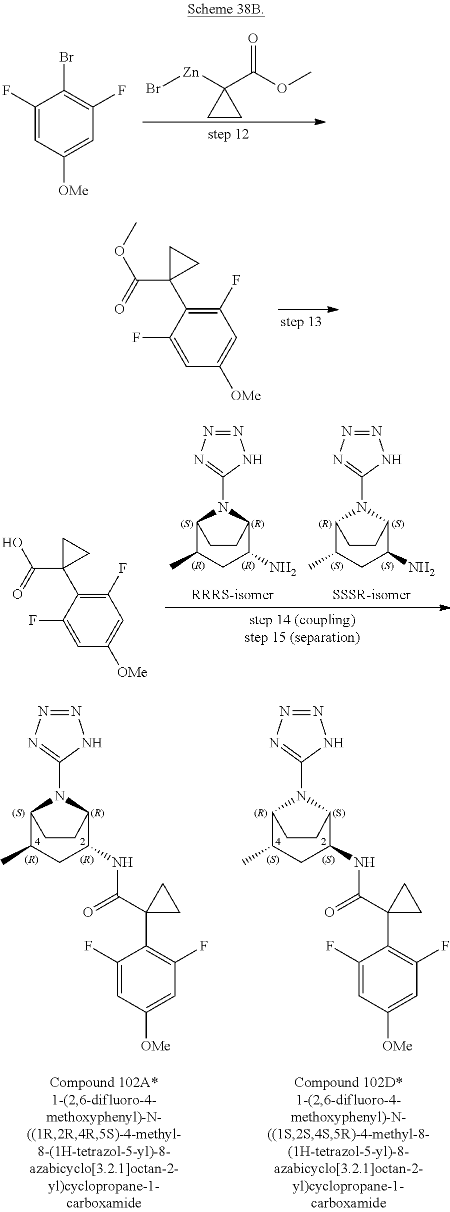

### Example 38 Follows Protocol A

Step 1: Into a 1 L 3-necked round-bottom flask were added 3-hydroxypyridine (50.0 g, 526 mmol, 1 equiv), isopropanol (IPA) (500 mL), and benzyl bromide (BnBr) (96.2 g, 562 mmol, 1.07 equiv) at room temperature. The resulting mixture was stirred overnight at 80° C. The reaction was monitored by LCMS. The resulting mixture was concentrated under reduced pressure. The residue was purified by washing with ethyl acetate (EtOAc) (200 mL). The solid was collected and dried over under vacuum. This resulted in 1-benzyl-3-hydroxypyridin-1-ium bromide (135 g, 97% yield). LCMS (ES, m/z): RT=0.47 min, m/z=186.1[M+H]⁺.

Step 2: To a stirred solution of 1-benzyl-3-hydroxypyridin-1-ium bromide (25.0 g, 93.9 mmol, 1 equiv), (ethenesulfonyl)benzene (21.0 g, 125 mmol, 1.33 equiv), hydroquinone (207 mg, 1.88 mmol, 0.02 equiv) in tetrahydrofuran (THF) (200 mL) was added triethylamine (NEt₃) (14.2 g, 141 mmol, 1.50 equiv) dropwise at room temperature under nitrogen atmosphere. The resulting mixture was stirred overnight at 65 C under nitrogen atmosphere. The reaction was monitored by LCMS. The reaction was quenched by the addition of water (300 mL) at room temperature. The aqueous layer was extracted with ethyl acetate (EtOAc) (3×200 mL). The organic phase was concentrated under reduced pressure and the solid was precipitated. The precipitated solid was collected and washed by EtOAc (3×50 mL). This resulted in a mixture of assumed (1R,5R,6S)-8-benzyl-6-(phenylsulfonyl)-8-azabicyclo[3.2.1]oct-3-en-2-one and the corresponding enantiomer (20 g, 60% yield). LCMS (ES, m/z): RT=0.91 min, m/z=354.1[M+H]⁺.

Step 3: To a stirred solution of the mixture of step 2 (20 g, 56.6 mmol, 1 equiv) in 200 mL tetrahydrofuran (THF) was added dimethyl copper lithium (Me₂CuLi) (0.5M in diethylether (Et₂O)) (169.8 mL, 84.88 mmol, 1.5 equiv) dropwise at −78° C. under nitrogen atmosphere. The resulting mixture was stirred for 1 h at room temperature under nitrogen atmosphere. The reaction was monitored by LCMS. The reaction was quenched by the addition of water (500 mL) at 0° C. The resulting mixture was extracted with ethyl acetate (EtOAc) (3×500 mL). The combined organic layers were washed with water (2×300 mL), dried over anhydrous Na₂SO₄, filtered, and the filtrate was concentrated under reduced pressure to provide a mixture of assumed (1R,4R,5R,6S)-8-benzyl-4-methyl-6-(phenylsulfonyl)-8-azabicyclo[3.2.1]octan-2-one and the corresponding enantiomer (18.0 g, 86% yield). LCMS (ES, m/z): RT=1.01 min, m/z=370.1[M+H]⁺.

Step 4: Into a 100 mL 3-necked round-bottom flask was added the mixture of step 3 (2.58 g, 6.98 mmol, 1 equiv) and hydroxylamine hydrochloride (534 mg, 7.68 mmol, 1.1 equiv), pyridine (1104 mg, 13.96 mmol, 2 equiv), and ethanol (EtOH) (25.8 mL) at room temperature. The resulting mixture was stirred overnight at room temperature. The reaction was monitored by LCMS. The reaction was quenched by the addition of water (100 mL) at room temperature. The resulting mixture was extracted with ethyl acetate (EtOAc) (3×100 mL). The combined organic layers were washed with water (2×50 mL), dried over anhydrous Na₂SO₄. After filtration, the filtrate was concentrated under reduced pressure. The solid was precipitated and precipitated solid was collected to provide a mixture of assumed N-[(1R,4R,5R,6S)-6-(benzenesulfonyl)-8-benzyl-4-methyl-8-azabicyclo[3.2.1]octan-2-ylidene]hydroxylamine and the corresponding enantiomer (2.5 g, 93% yield). LCMS (ES, m/z): RT=0.78 min, m/z=385.1[M+H]⁺.

Step 5: A solution of the mixture of step 4 (2.50 g, 6.50 mmol, 1 equiv) in dimethylformamide (DMF) was treated with NaBH₃CN (1.63 g, 26.0 mmol, 4 equiv), sodium bisulfate (2.34 g, 19.5 mmol, 3.00 equiv) for 5 min at room temperature under nitrogen atmosphere followed by the addition of molybdenum pentachloride (0.89 g, 3.25 mmol, 0.5 equiv) in portions at room temperature. The resulting mixture was stirred overnight at room temperature under nitrogen atmosphere. The reaction was monitored by LCMS. The reaction was quenched by the addition of sat. NaHCO₃ (aq.) (100 mL) at room temperature. The resulting mixture was extracted with ethyl acetate (EtOAc) (3×100 mL). The combined organic layers were washed with water (2×50 mL) and dried over anhydrous Na₂SO₄. After filtration, the filtrate was concentrated under reduced pressure to provide a mixture of assumed (1R,2R,4R,5R,6S)-8-benzyl-4-methyl-6-(phenylsulfonyl)-8-azabicyclo[3.2.1]octan-2-amine and the corresponding enantiomer (1.80 g, 75% yield). LCMS (ES, m/z): RT=0.65 min, m/z=371.1[M+H]⁺.

Step 6: A solution of the mixture of step 5 (1 g, 2.69 mmol, 1 equiv) and magnesium chips (treated with 0.5% HCl) (0.52 g, 22 mmol, 7.9 equiv) in methanol (MeOH) (15 mL) was stirred overnight at room temperature. To the above mixture was added acetic acid (AcOH) (0.66 mL) and H₂O (10 mL) at room temperature. The resulting mixture was stirred for additional 1h at room temperature. The reaction was monitored by LCMS. The reaction was quenched by the addition of sat. NaOH (aq.) (5 mL) at room temperature. The resulting mixture was extracted with ethyl acetate (EtOAc) (3×10 mL), and the combined organic layers were washed with water (3×10 mL), dried over anhydrous Na₂SO₄, filtered, and the filtrate was concentrated under reduced pressure to provide a mixture of assumed (1R,2R,4R,5S)-8-benzyl-4-methyl-8-azabicyclo[3.2.1]octan-2-amine and the corresponding enantiomer (700 mg, 84% yield). LCMS (ES, m/z): RT=0.34 min, m/z=231.1[M+H]⁺.

Step 7: Into an 8 mL vial was added the mixture of step 6 (700 mg, 3.03 mmol, 1.00 equiv), methanol (MeOH) (3 mL), and ditertbutyldicarbonate (Boc₂O) (796 mg, 3.64 mmol, 1.20 equiv) at room temperature. The resulting mixture was stirred at room temperature for 2 h. The reaction was monitored by LCMS. The resulting mixture was concentrated under reduced pressure. The residue was purified by silica gel column chromatography, eluted with petroleum ether/ethyl acetate (5:1) to afford a mixture of assumed tert-butyl ((1R,2R,4R,5S)-8-benzyl-4-methyl-8-azabicyclo[3.2.1]octan-2-yl)carbamate and the corresponding enantiomer (800 mg, 80% yield). LCMS (ES, m/z): RT=0.62 min, m/z=331.1[M+H]⁺.

Step 8: To a stirred solution of the mixture of step 7 (4.30 g, 13.0 mmol, 1 equiv) in isopropanol (i-PrOH) (50 mL) was added Pd/C (wet, 10% on carbon, 2.76 g) at room temperature. The resulting mixture was stirred for 16 h at room temperature under hydrogen atmosphere. The reaction was monitored by LCMS. The resulting mixture was filtered, the filter cake was washed with methanol (MeOH) (3×50 mL). The filtrate was concentrated under reduced pressure to afford a mixture of assumed tert-butyl ((1R,2R,4R,5S)-4-methyl-8-azabicyclo[3.2.1]octan-2-yl)carbamate and the corresponding enantiomer (2.7 g, 86% yield). LCMS (ES, m/z): RT=0.50 min, m/z=241.2[M+H]⁺.

Step 9: To a stirred solution of the mixture of step 8 (2.70 g, 11.2 mmol, 1 equiv) and K₂CO₃ (4.69 g, 33.7 mmol, 3.00 equiv) in acetonitrile (30 mL) was added cyanogen bromide (4.76 g, 44.9 mmol, 4.00 equiv) at room temperature. The resulting mixture was stirred for 2 h at 80° C. The reaction was monitored by LCMS. The reaction was quenched with water (50 mL) at room temperature. The resulting mixture was extracted with ethyl acetate (EtOAc) (2×100 mL). The combined organic layers were washed with brine (1×50 mL), dried over anhydrous Na₂SO₄, filtered, and the filtrate was concentrated under reduced pressure to afford a mixture of assumed tert-butyl ((1R,2R,4R,5S)-8-cyano-4-methyl-8-azabicyclo[3.2.1]octan-2-yl)carbamate and the corresponding enantiomer (2.7 g, 91% yield). LCMS (ES, m/z): RT=0.66 min, m/z=266.2[M+H]⁺.

Step 10: To a stirred solution of the mixture of step 9 (1.30 g, 4.89 mmol, 1 equiv) and dibutyltin oxide (Bu₂SnO) (2.43 g, 9.79 mmol, 2 equiv) in dimethylformamide (DMF) (13 mL) was added trimethylsilyl azide (1.12 g, 9.79 mmol, 2 equiv) at room temperature. The resulting mixture was stirred for 2 h at 120° C. The reaction was monitored by LCMS. The residue was purified by reversed-phase flash chromatography (C18 silica gel; mobile phase, MeCN in water (0.1% TFA), 30% to 50% gradient in 10 min; detector, UV 254/220 nm) to afford a mixture of assumed tert-butyl ((1R,2R,4R,5S)-4-methyl-8-(1H-tetrazol-5-yl)-8-azabicyclo[3.2.1]octan-2-yl)carbamate and the corresponding enantiomer (0.90 g, 60% yield). LCMS (ES, m/z): RT=0.67 min, m/z=309.1[M+H]⁺.

Step 11: Into a 250 mL round-bottom flask was added the mixture of step 10 (0.90 g, 2.9 mmol, 1.0 equiv) and HCl (gas) in 1,4-dioxane (4M, 10.0 mL) at room temperature. The resulting mixture was stirred for 2 h at room temperature. The reaction was monitored by LCMS. The resulting mixture was concentrated under reduced pressure to afford a mixture of assumed (1R,2R,4R,5S)-4-methyl-8-(1H-tetrazol-5-yl)-8-azabicyclo[3.2.1]octan-2-amine hydrochloride salt (RRRS-isomer) and (1S,2S,4S,5R)-4-methyl-8-(1H-tetrazol-5-yl)-8-azabicyclo[3.2.1]octan-2-amine hydrochloride salt (SSSR-isomer) (0.80 g, 95% yield), collectively referred to as the “amine (i) reagent”. LCMS (ES, m/z): RT=0.15 min, m/z=209.1[M+H]⁺.

Step 12: Into a 40 mL vial were added 2-bromo-1,3-difluoro-5-methoxybenzene (500 mg, 2.24 mmol, 1 equiv) and bromo[1-(methoxycarbonyl)cyclopropyl]zinc (0.93M in THF, 20 mL, 11.2 mmol, 8 equiv), and bis(tri-tert-butylphosphane) palladium (573 mg, 1.12 mmol, 0.5 equiv). The resulting mixture was stirred for 1 h at 60° C. under nitrogen atmosphere. The reaction was monitored by LCMS. The resulting mixture was filtered, the filter cake was washed with dichloromethane (DCM) (3×20 mL). The filtrate was concentrated under reduced pressure. The residue was purified by silica gel column chromatography, eluted with DCM/petroleum ether (1:3) to afford methyl 1-(2,6-difluoro-4-methoxyphenyl)cyclopropane-1-carboxylate (300 mg, 55% yield). LCMS (ES, m/z): RT=0.85 min, m/z=243.1[M+H]⁺.

Step 13: Into an 8 mL vial were added methyl 1-(2,6-difluoro-4-methoxyphenyl)cyclopropane-1-carboxylate (200 mg, 0.83 mmol, 1 equiv), methanol (2 mL), NaOH (165 mg, 4.13 mmol, 5 equiv), and water (0.4 mL). The resulting mixture was stirred for 2 h at 60° C. The reaction was monitored by LCMS. The mixture was adjusted to pH=4 with 2 M HCl. The resulting mixture was extracted with ethyl acetate (EtOAc) (3×20 mL). The combined organic layers were washed with brine (10 mL), and dried over anhydrous Na₂SO₄. After filtration, the filtrate was concentrated under reduced pressure to afford 1-(2,6-difluoro-4-methoxyphenyl)cyclopropane-1-carboxylic acid (150 mg, 80% yield), (“carboxyl (ii) reagent”). LCMS (ES, m/z): RT=0.72 min, m/z=229.1[M+H]⁺.

Step 14: Into an 8 mL vial were added the carboxyl (ii) reagent from step 13 (100 mg, 0.44 mmol, 1 equiv), dimethylformamide (2 mL), the amine (i) reagent” from step 11 (91.3 mg, 0.44 mmol, 1 equiv), hydroxybenzotriazole (HOBT) (71.1 mg, 0.53 mmol, 1.20 equiv), I-ethyl-3-(3-dimethylaninopropyl)carbodiimide (EDCI) (126 mg, 0.66 mmol, 1.5 equiv), and diisopropylethyl amine (DIEA) (170 mg, 1.31 mmol, 3 equiv). The resulting mixture was stirred for 1 h at room temperature. The reaction was monitored by LCMS. The reaction was quenched with water (20 mL) at room temperature. The resulting mixture was extracted with ethyl acetate (EtOAc) (3×20 mL). The combined organic layers were washed with brine (10 mL), dried over anhydrous Na₂SO₄, filtered, and the filtrate was concentrated under reduced pressure to provide a residue, which was purified by reversed-phase flash chromatography (C18 silica gel; mobile phase, MeCN in water (0.1% formic acid), 40% to 60% gradient in 10 min; detector, UV 254/220 nm) to afford 1-(2,6-difluoro-4-methoxyphenyl)-N-((1R,4R,5S)-4-methyl-8-(1H-tetrazol-5-yl)-8-azabicyclo[3.2.1]octan-2-yl)cyclopropane-1-carboxamide (60 mg, 80% purity). The crude product was purified by Prep-HPLC (Xselect CSH C18 OBD Column 30*150 mm, 5 μm; Mobile Phase A: water (0.1% formic acid), Mobile Phase B: acetonitrile; Flow rate: 60 mL/min; Gradient: 5% B to 5% B in 2 min, 35% B to 45% B in 10 min; Wave Length: 254/220 nm; RT(min): 9.2) to afford a mixture of assumed 1-(2,6-difluoro-4-methoxyphenyl)-N-((1R,2R,4R,5S)-4-methyl-8-(1H-tetrazol-5-yl)-8-azabicyclo[3.2.1]octan-2-yl)cyclopropane-1-carboxamide (Compound 102A*) and 1-(2,6-difluoro-4-methoxyphenyl)-N-((1S,2S,4S,5R)-4-methyl-8-(1H-tetrazol-5-yl)-8-azabicyclo[3.2.1]octan-2-yl)cyclopropane-1-carboxamide (Compound 102D*) (40 mg). LCMS (ES, m/z): RT=1.61 min, m/z=419.2[M+H]⁺.

Step 15: The mixture of step 14 (40 mg, 98% purity) was separated by Prep-Chiral-HPLC (CHIRALPAK SC 2*25 cm, 5 μm; Mobile Phase A: Hexanes (0.1% TFA), Mobile Phase B: (EtOH:DCM=1:1); Flow rate: 20 mL/min; Gradient: isocratic 20) to afford Compound 102A* (13.3 mg, RT(min): 15.53) as the first eluting peak, and Compound 102D* (12.7 mg, RT(min): 21.33) as the second eluting peak. Stereochemistry was arbitrarily assigned.

Compound 102A*: LCMS (ES, m/z): RT=0.75 min, m/z=419.2[M+H]⁺. ¹H NMR (400 MHz, Methanol-d₄) δ 6.74-6.56 (m, 2H), 4.24-4.14 (m, 2H), 4.13-4.06 (m, 1H), 3.84 (s, 3H), 2.05-1.83 (m, 2H), 1.81-1.58 (m, 6H), 1.47-1.35 (m, 1H), 1.12 (t, J=5.6 Hz, 5H).

Compound 102D*: LCMS (ES, m/z): RT=0.75 min, m/z=419.2[M+H]⁺. ¹H NMR (400 MHz, Methanol-d₄) δ 6.77-6.58 (m, 2H), 4.26-4.14 (m, 2H), 4.14-4.06 (m, 1H), 3.84 (s, 3H), 2.11-1.81 (m, 2H), 1.80-1.57 (m, 6H), 1.47-1.34 (m, 1H), 1.22-1.01 (m, 5H).

### Example 39. N-[(1R,2R,4R,5S)-8-(1H-1,2,3,4-tetrazol-5-yl)-4-(trifluoromethyl)-8-azabicyclo[3.2.1]octan-2-yl]-1-(4-chlorophenyl)cyclopropane-1-carboxamide (Compound 103A*), N-[(1R,2S,4R,5S)-8-(1H-1,2,3,4-tetrazol-5-yl)-4-(trifluoromethyl)-8-azabicyclo[3.2.1]octan-2-yl]-1-(4-chlorophenyl)cyclopropane-1-carboxamide (Compound 103B*), N-[(1S,2R,4S,5R)-8-(1H-1,2,3,4-tetrazol-5-yl)-4-(trifluoromethyl)-8-azabicyclo[3.2.1]octan-2-yl]-1-(4-chlorophenyl)cyclopropane-1-carboxamide (Compound 103C*) and N-[(1S,2S,4S,5R)-8-(1H-1,2,3,4-tetrazol-5-yl)-4-(trifluoromethyl)-8-azabicyclo[3.2.1]octan-2-yl]-1-(4-chlorophenyl)cyclopropane-1-carboxamide (Compound 103D*)

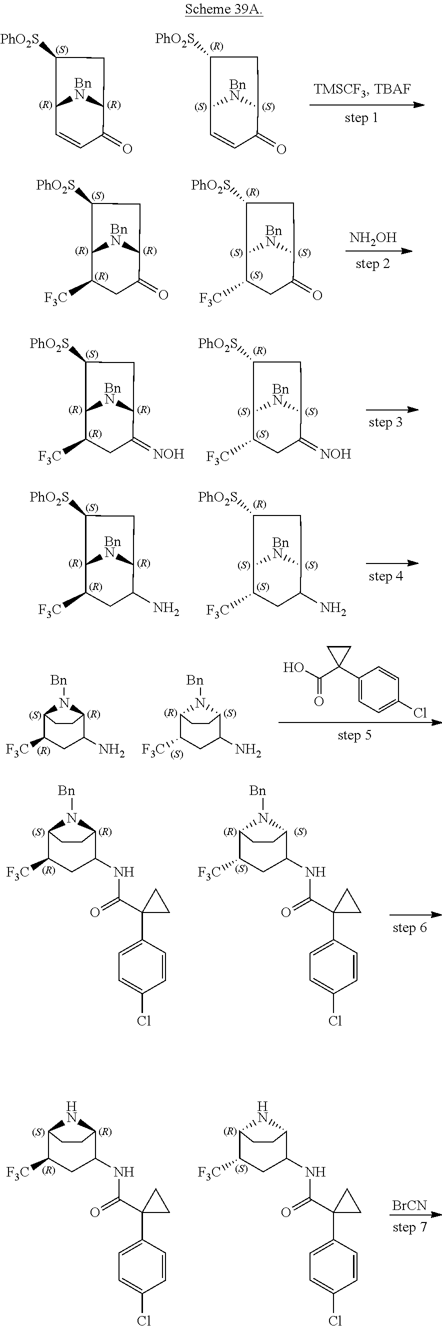

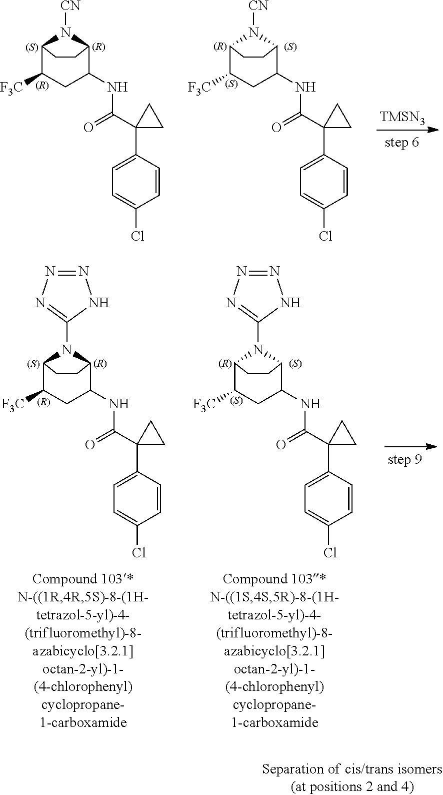

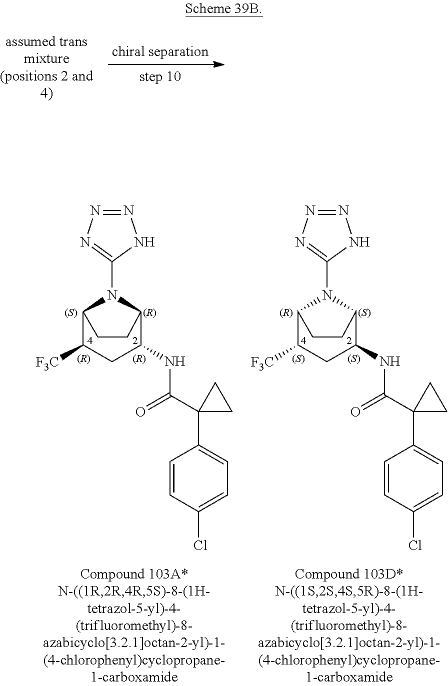

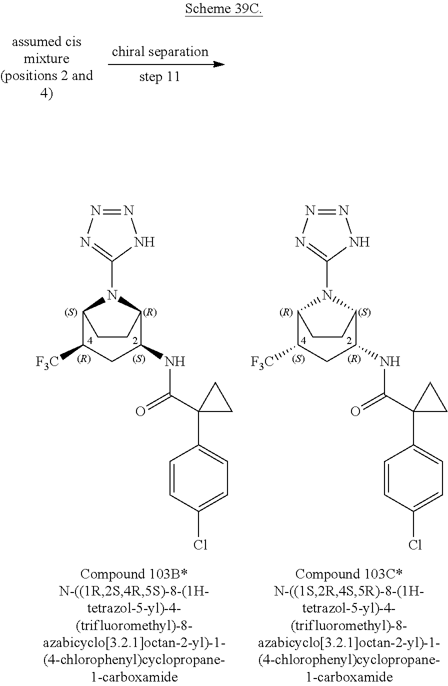

### Example 39 Follows Protocol C

Step 1: A mixture of assumed (1R,5R,6S)-6-(benzenesulfonyl)-8-benzyl-8-azabicyclo[3.2.1]oct-3-en-2-one and the corresponding enantiomer (from Example 38, step 2) (2 g, 5.65 mmol, 1 equiv) in tetrahydrofuran (100 mL) was treated with trifluoromethyltrimethylsilane (1.61 g, 11.31 mmol, 2.00 equiv) for 3 min at 0° C. under nitrogen atmosphere followed by the dropwise addition of tetrabutylammonium fluoride (TBAF, 1M in THF) (5.66 mL, 5.65 mmol, 1 equiv). The resulting mixture was stirred for 1 h at 0° C. under nitrogen atmosphere followed by the addition of 2M HCl (20 mL) at 0° C., then was stirred for 1 h at room temperature and quenched by the addition of water (100 mL).

The mixture was extracted with ethyl acetate (EtOAc) (3×60 mL) and the combined organic layers were concentrated under reduced pressure. The residue was purified by reversed-phase flash chromatography (C18 silica gel; mobile phase, acetonitrile in water, 50% to 60% gradient in 10 min; detector, UV 220 nm) to provide assumed (1R,5R,6S)-6-(benzenesulfonyl)-8-benzyl-4-(trifluoromethyl)-8-azabicyclo[3.2.1]octan-2-one and the corresponding enantiomer (200 mg, 8% yield). LCMS (ES, m/z): RT=0.89 min, m/z=424.1 [M+H]⁺.

Step 2: Into a 40 mL vial were added a mixture of assumed (1R,5R,6S)-6-(benzenesulfonyl)-8-benzyl-4-(trifluoromethyl)-8-azabicyclo[3.2.1]octan-2-one and the corresponding enantiomer (800 mg, 1.88 mmol, 1 equiv), hydroxylamine hydrochloride (144.41 mg, 2.07 mmol, 1.1 equiv), and pyridine (448.32 mg, 5.66 mmol, 3 equiv) in 10 mL of ethanol at room temperature. The resulting mixture was stirred overnight at room temperature, then quenched by the addition of water (100 mL) at room temperature. The mixture was extracted with ethyl acetate (EtOAc) (3×50 mL) and the combined organic layers were concentrated under reduced pressure. The residue was purified by silica gel column chromatography, eluting with petroleum ether/ethyl acetate (3:2), to afford a mixture of assumed N-((1R,2Z,5R,6S)-6-(benzenesulfonyl)-8-benzyl-4-(trifluoromethyl)-8-azabicyclo[3.2.1]octan-2-ylidene)hydroxylamine and the corresponding enantiomer (700 mg, 85% yield). LCMS (ES, m/z): RT=0.84 min, m/z=439.1[M+H]⁺.

Step 3: A mixture of assumed N-((1R,2Z,5R,6S)-6-(benzenesulfonyl)-8-benzyl-4-(trifluoromethyl)-8-azabicyclo[3.2.1]octan-2-ylidene)hydroxylamine and the corresponding enantiomer (700 mg, 1.59 mmol, 1 equiv) in dimethylformamide (5 mL) was treated with NaBH₃CN (401.29 mg, 6.38 mmol, 4 equiv) and sodium bisulfate (574.97 mg, 4.78 mmol, 3 equiv) for 3 min at 0° C. under nitrogen atmosphere followed by the addition of molybdenum pentachloride (43.62 mg, 0.16 mmol, 0.10 equiv) at 0° C. The resulting mixture was stirred for 2 h at 0° C. under nitrogen atmosphere and quenched with water (40 ml) at room temperature. The mixture was extracted with ethyl acetate (EtOAc) (3×50 mL) and the combined organic layers were concentrated under reduced pressure. The residue was purified by silica gel column chromatography, eluting with petroleum ether/ethyl acetate (1:1), to afford a mixture of assumed (1R,5R,6S)-6-(benzenesulfonyl)-8-benzyl-4-(trifluoromethyl)-8-azabicyclo[3.2.1]octan-2-amine and the corresponding enantiomer (500 mg, 74% yield). LCMS (ES, m/z): RT=0.65 min, m/z=425.1[M+H]⁺.

Step 4: Into a 40 mL vial were added a mixture of assumed (1R,5R,6S)-6-(benzenesulfonyl)-8-benzyl-4-(trifluoromethyl)-8-azabicyclo[3.2.1]octan-2-amine and the corresponding enantiomer (500 mg, 1.17 mmol, 1 equiv) and Mg powder (286.29 mg, 11.78 mmol, 10 equiv), in methanol (MeOH) (25 mL) at room temperature. The resulting mixture was stirred for 2 h at room temperature, then was quenched with water (50 ml). The mixture was extracted with ethyl acetate (EtOAc) (3×150 mL). The combined organic layers were concentrated under reduced pressure. The residue was purified by silica gel column chromatography, eluting with petroleum ether/ethyl acetate (1:1), to afford a mixture of assumed (1R,5S)-8-benzyl-4-(trifluoromethyl)-8-azabicyclo[3.2.1]octan-2-amine and the corresponding enantiomer (300 mg, 90% yield), collectively referred to as the “amine (iv) reagent”. LCMS (ES, m/z): RT=0.53 min, m/z=285.1[M+H]⁺.

Step 5: Into a 20 mL vial were added the amine (iv) reagent from step 4 (280 mg, 0.98 mmol, 1 equiv), 1-(4-chlorophenyl)cyclopropane-1-carboxylic acid (carboxyl (ii) reagent) (193.64 mg, 0.98 mmol, 1 equiv), hexafluorophosphate azabenzotriazole tetramethyl uronium (HATU) (561.67 mg, 1.40 mmol, 1.5 equiv) and triethylamine (298.96 mg, 2.95 mmol, 3 equiv) in 5 mL of dimethylformamide (DMF) at room temperature. The resulting mixture was stirred for 2 h at room temperature, then was quenched by the addition of water (50 mL). The mixture was extracted with ethyl acetate (EtOAc) (3×50 mL) and the combined organic layers were concentrated under reduced pressure. The residue was purified by reversed-phase flash chromatography (C18 silica gel; mobile phase, acetonitrile in water, 40% to 50% gradient in 10 min; detector, UV 220 nm) to provide a mixture of assumed N-[(1R,5S)-8-benzyl-4-(trifluoromethyl)-8-azabicyclo[3.2.1]octan-2-yl]-1-(4-chlorophenyl)cyclopropane-1-carboxamide and the corresponding enantiomer (170 mg, 37% yield). LCMS (ES, m/z): RT=0.71 min, m/z=463.1[M+H]⁺.

Step 6: A solution of a mixture of assumed N-[(1R,5S)-8-benzyl-4-(trifluoromethyl)-8-azabicyclo[3.2.1]octan-2-yl]-1-(4-chlorophenyl)cyclopropane-1-carboxamide and the corresponding enantiomer (150 mg, 0.32 mmol, 1 equiv), Pd/C (172.41 mg, 10% on carbon) and ZnBr₂ (80.26 mg, 0.35 mmol, 1.1 equiv) in 5 mL of methanol was stirred for 2 h at room temperature under hydrogen atmosphere. The reaction was monitored by LCMS, filtered, and the filter cake was washed with methanol (3×10 mL). The filtrate was concentrated under reduced pressure to provide a mixture of assumed 1-(4-chlorophenyl)-N-[(1R,5S)-4-(trifluoromethyl)-8-azabicyclo[3.2.1]octan-2-yl]cyclopropane-1-carboxamide and the corresponding enantiomer (100 mg, 83% yield). LCMS (ES, m/z): RT=0.61 min, m/z=373.1[M+H]⁺.

Step 7: Into an 8 mL vial were added a mixture of assumed 1-(4-chlorophenyl)-N-[(1R,5S)-4-(trifluoromethyl)-8-azabicyclo[3.2.1]octan-2-yl]cyclopropane-1-carboxamide and the corresponding enantiomer (100 mg, 0.26 mmol, 1 equiv), BrCN (42.62 mg, 0.40 mmol, 1.5 equiv) and K₂CO₃ (111.21 mg, 0.80 mmol, 3 equiv) in 3 mL of acetonitrile at room temperature. The resulting mixture was stirred for 1 h at room temperature, then quenched by the addition of water (20 mL). The resulting mixture was extracted with ethyl acetate (EtOAc) (3×20 mL). The combined organic layers were washed with water (2×10 mL), dried over anhydrous Na₂SO₄, filtered, and concentrated under reduced pressure.

This resulted in a mixture of assumed 1-(4-chlorophenyl)-N-[(1R,5S)-8-cyano-4-(trifluoromethyl)-8-azabicyclo[3.2.1]octan-2-yl]cyclopropane-1-carboxamide and the corresponding enantiomer (90 mg, 84% yield). LCMS (ES, m/z): RT=0.81 min, m/z=398.1[M+H]⁺.

Step 8: Into an 8 mL vial were added a mixture of assumed 1-(4-chlorophenyl)-N-[(1R,5S)-8-cyano-4-(trifluoromethyl)-8-azabicyclo[3.2.1]octan-2-yl]cyclopropane-1-carboxamide and the corresponding enantiomer (90 mg, 0.22 mmol, 1 equiv), NH₄C4 (36.30 mg, 0.67 mmol, 3 equiv) and azidotrimethylsilane (78.19 mg, 0.67 mmol, 3 equiv) in 2 mL of dimethylformamide at room temperature. The resulting mixture was stirred for 2 h at 120° C. under nitrogen atmosphere. The reaction was monitored by LCMS and was quenched by the addition of water (1 mL) at room temperature. The resulting mixture was concentrated under vacuum to provide a mixture of Compound 103′* and Compound 103″*.

Step 9: The residue from step 8, a mixture of Compound 103′* and Compound 103″*, was purified by reversed-phase flash chromatography (C18 silica gel; mobile phase, acetonitrile in water, 40% to 45% gradient in 20 min; detector, UV 220 nm) to provide as the first eluting peak an assumed mixture of cis isomers (at positions 2 and 4) (10 mg, 10% yield, LCMS (ES, m/z): RT=0.70 min, m/z=441.1[M+H]⁺), and the second eluting peak an assumed mixture of trans isomers (at positions 2 and 4) (30 mg, 29% yield, LCMS (ES, m/z): RT=0.77 min, m/z=441.1[M+H]⁺).

Step 10: The assumed mixture of trans isomers from step 9 (30 mg) was purified by Prep-Chiral-HPLC (CHIRALART Cellulose-SC, 2*25 cm, 5 μm; Mobile Phase A: hexanes (0.1% trifluoroacetic acid), Mobile Phase B: ethanol:dichloromethane=1:1; Flow rate: 20 mL/min; Gradient: isocratic 25; Wave Length: 220/254 nm) to afford N-[(1R,2R,4R,5S)-8-(1H-1,2,3,4-tetrazol-5-yl)-4-(trifluoromethyl)-8-azabicyclo[3.2.1]octan-2-yl]-1-(4-chlorophenyl)cyclopropane-1-carboxamide (Compound 103A*) (15.2 mg, 49% yield, RT(min): 7.2) as the first eluting peak, and N-[(1S,2S,4S,5R)-8-(1H-1,2,3,4-tetrazol-5-yl)-4-(trifluoromethyl)-8-azabicyclo[3.2.1]octan-2-yl]-1-(4-chlorophenyl)cyclopropane-1-carboxamide (Compound 103D*) (13.7 mg, 45% yield, RT(min): 8.6) as the second eluting peak. Stereochemistry was arbitrarily assigned.

Compound 103A*: LCMS (ES, m/z): RT=0.88 min, m/z=441.1[M+H]⁺. ¹H NMR (400 MHz, Methanol-d₄) δ 7.42 (s, 4H), 4.71-4.65 (m, 1H), 4.28 (d, J=12.6 Hz, 2H), 2.55 (q, J=9.4 Hz, 1H), 2.13-2.01 (m, 1H), 1.87 (d, J=6.4 Hz, 2H), 1.86-1.71 (m, 3H), 1.59-1.48 (m, 2H), 1.22-1.09 (m, 2H).

Compound 103D*: LCMS (ES, m/z): RT=0.72 min, m/z=441.1[M+H]⁺. ¹H NMR (400 MHz, Methanol-d₄) δ 7.42 (s, 4H), 4.68 (dd, J=7.0, 1.8 Hz, 1H), 4.28 (d, J=12.8 Hz, 2H), 2.56 (p, J=9.8 Hz, 1H), 2.13-2.03 (m, 1H), 1.84 (dd, J=22.0, 6.4 Hz, 2H), 1.76 (s, 1H), 1.75 (q, J=4.9, 3.6 Hz, 2H), 1.61-1.48 (m, 2H), 1.22-1.09 (m, 2H).

Step 11: The assumed mixture of cis isomers from step 9 (10 mg) was purified by Prep-Chiral-HPLC (CHIRALPAK IE, 2*25 cm, 5 μm; Mobile Phase A: Mobile Phase A: hexanes (0.1% trifluoroacetic acid), Mobile Phase B: ethanol:dichloromethane=1:1; Flow rate: 20 mL/min; Gradient: isocratic 25; Wave Length: 220/254 nm) afford N-[(1R,2S,4R,5S)-8-(1H-1,2,3,4-tetrazol-5-yl)-4-(trifluoromethyl)-8-azabicyclo[3.2.1]octan-2-yl]-1-(4-chlorophenyl)cyclopropane-1-carboxamide (Compound 103B*) (3.0 mg, 29% yield, RT(min): 6.54) as the first eluting peak, and N-[(1S,2R,4S,5R)-8-(1H-1,2,3,4-tetrazol-5-yl)-4-(trifluoromethyl)-8-azabicyclo[3.2.1]octan-2-yl]-1-(4-chlorophenyl)cyclopropane-1-carboxamide (Compound 103C*) (3.9 mg, 49% yield, RT(min): 9.3) as the second eluting peak. Stereochemistry was arbitrarily assigned.

Compound 103B*: LCMS (ES, m/z): RT=0.64 min, m/z=441.1[M+H]⁺. ¹H NMR (400 MHz, Methanol-d₄) δ 7.33-7.25 (m, 2H), 7.21-7.14 (m, 2H), 4.52 (d, J=6.8 Hz, 1H), 4.24 (d, J=6.3 Hz, 1H), 4.08-4.02 (m, 1H), 2.48 (p, J=10.4 Hz, 1H), 2.32 (dd, J=15.4, 7.4 Hz, 1H), 2.12-1.84 (m, 2H), 1.71-1.56 (m, 2H), 1.50 (dd, J=9.8, 3.6 Hz, 1H), 1.17 (dd, J=10.2, 3.6 Hz, 1H), 1.04 (dd, J=9.8, 3.6 Hz, 1H).

Compound 103C*: LCMS (ES, m/z): RT=0.80 min, m/z=441.1[M+H]⁺. ¹H NMR (400 MHz, Methanol-d₄) δ 7.33-7.25 (m, 2H), 7.21-7.14 (m, 2H), 4.52 (d, J=6.8 Hz, 1H), 4.28-4.21 (m, 1H), 4.04 (t, J=4.4 Hz, 1H), 2.48 (p, J=10.4 Hz, 1H), 2.32 (dd, J=15.4, 7.2 Hz, 1H), 2.16-1.94 (m, 2H), 1.90 (dd, J=10.0, 7.4 Hz, 2H), 1.71-1.56 (m, 2H), 1.50 (dd, J=9.8, 3.6 Hz, 1H), 1.17 (dd, J=10.2, 3.6 Hz, 1H), 1.03 (dd, J=9.8, 3.4 Hz, 1H).

### Example 40. N-(4-methyl-9-(1H-tetrazol-5-yl)-9-azabicyclo[4.2.1]nonan-2-yl)-1-(4-(trifluoromethyl)phenyl)cyclopropane-1-carboxamide (Compound 104, rac-104), and Compounds 104A*, 104B*, 104C*, 104D*, 104E*, 104F*, 104G* and 104H*

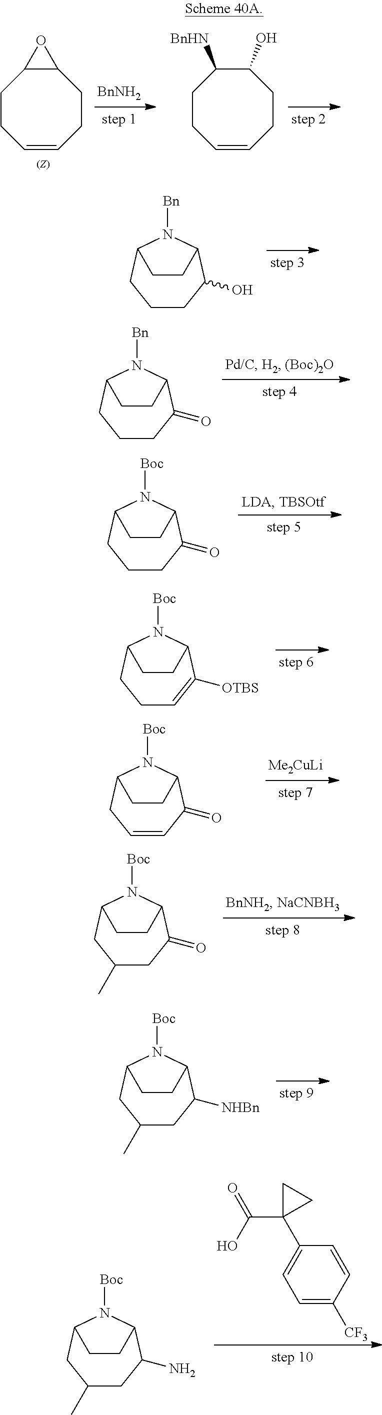

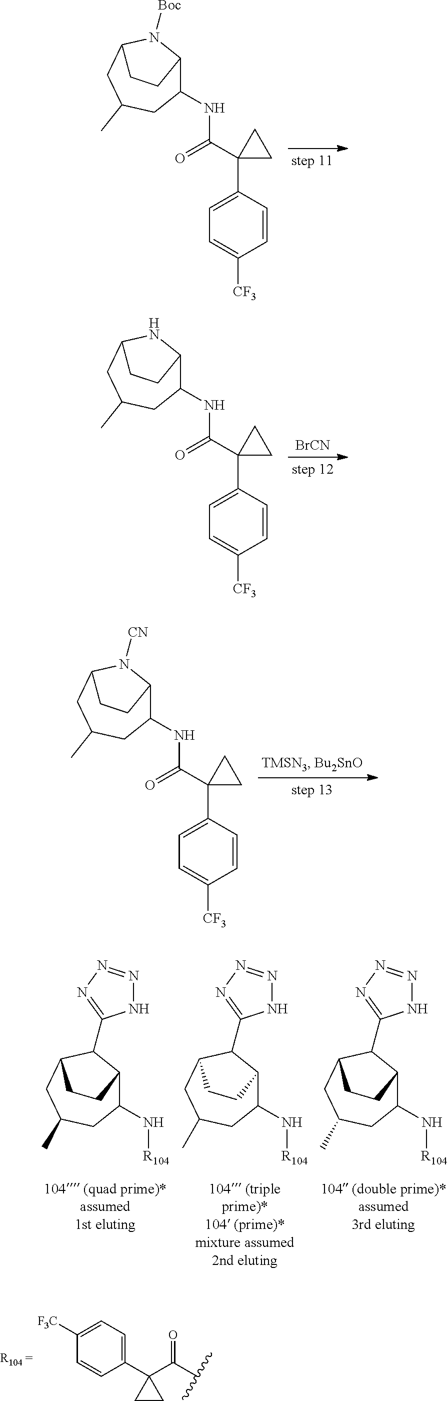

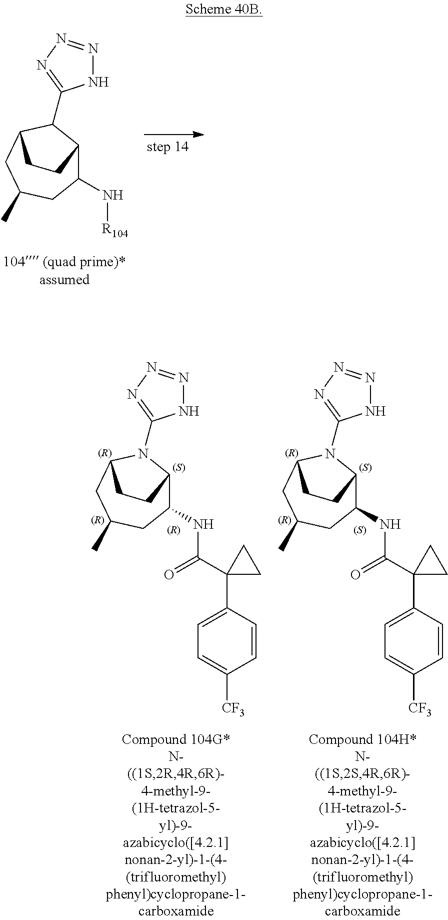

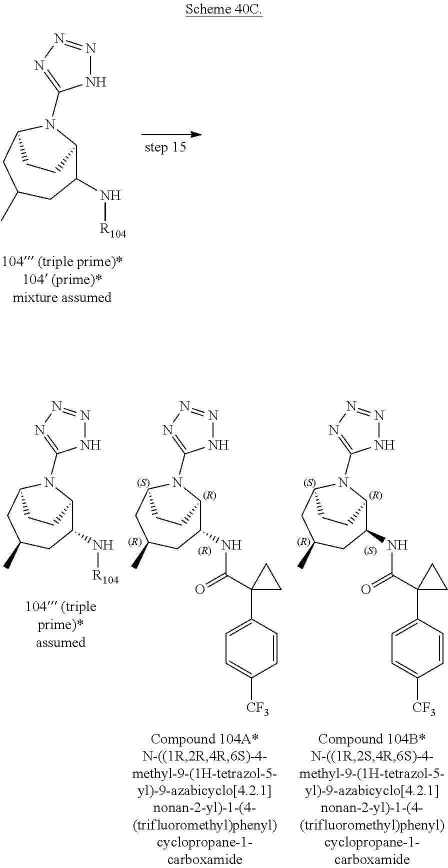

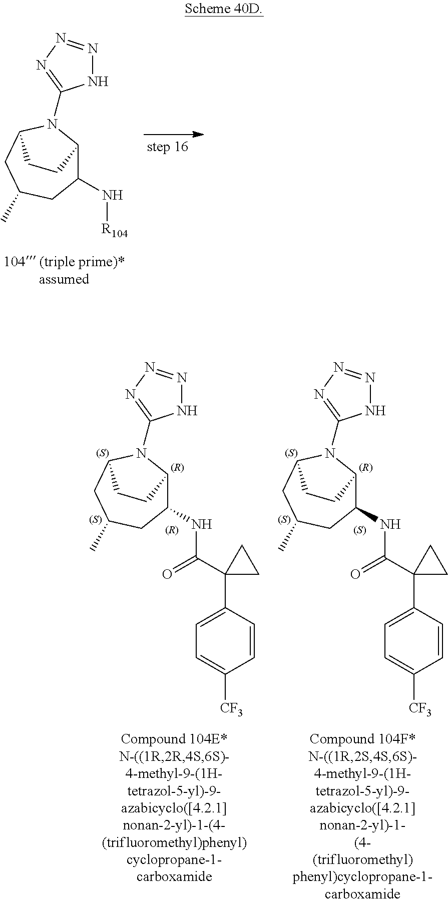

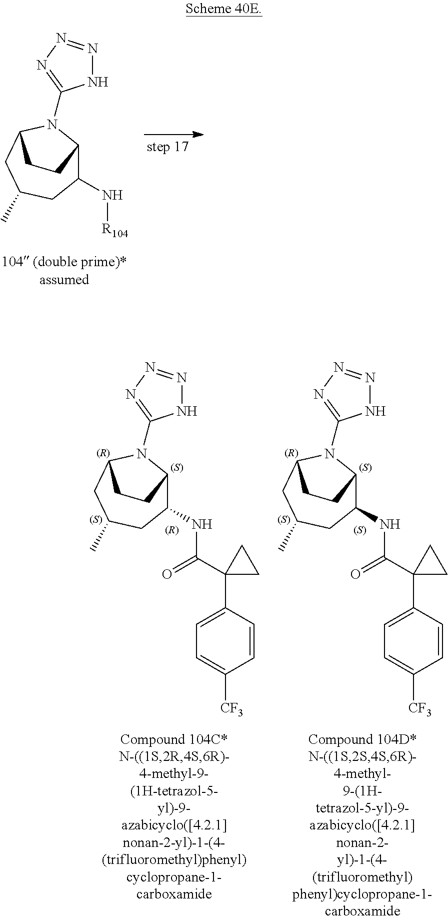

### Example 40 Follows Protocol C

Step 1: A solution of (4Z)-9-oxabicyclo[6.1.0]non-4-ene (50 g, 402.32 mmol, 1 equiv) and benzylamine (129.4 g, 1207.78 mmol, 3 equiv) in methanol (500 mL) was stirred for 12 h at 80° C. The reaction was monitored by LCMS, concentrated under reduced pressure and the residue was purified by reversed-phase flash chromatography (C18 silica gel; mobile phase, acetonitrile in water, 10% to 50% gradient in 10 min; detector, UV 254 nm) to provide 1,8-anti-(4Z)-8-(benzylamino) cyclooct-4-en-1-ol (23 g, 25% yield). LCMS (ES, m/z): RT=0.85 min, m/z=232.2[M+H]⁺.

Step 2: A solution of mercuric acetate (30 g, 98.49 mmol, 1.2 equiv) in tetrahydrofuran (THF) (250 mL) and H₂O (250 mL) was treated with 1,8-anti-(4Z)-8-(benzylamino) cyclooct-4-en-1-ol (23 g, 82.08 mmol, 1 equiv). The resulting mixture was stirred for 30 min at 0° C. followed by the dropwise addition of a solution of NaBH₄ (8 g, 202.96 mmol, 2.35 equiv) in NaOH (3M) (70 mL) at 0° C. The resulting mixture was stirred for an additional 15 min at 0° C. and additional 15 min at room temperature.

The reaction was monitored by LCMS, diluted with water (600 mL) and extracted with ethyl acetate (EtOAc) (3×300 mL). The combined organic layers were washed with brine (1×300 mL), dried over anhydrous Na₂SO₄, filtered, and concentrated under reduced pressure. The residue was purified by silica gel column chromatography, eluting with petroleum ether/ethyl acetate (2:1), to afford 9-benzyl-9-azabicyclo[4.2.1]nonan-2-ol (16.7 g, 73% yield). LCMS (ES, m/z): RT=0.92 min, m/z=232.2[M+H]⁺.

Step 3: A solution of 9-benzyl-9-azabicyclo[4.2.1]nonan-2-ol (16.7 g, 71.52 mmol, 1 equiv) and 2-iodoxybenzoic acid (IBX) (40.00 g, 143.11 mmol, 2 equiv) in ethyl acetate (EtOAc) (360 mL) was stirred for 2 h at 60° C. The reaction was monitored by LCMS, filtered, and the filter cake was washed with ethyl acetate (EtOAc) (2×150 mL). The filtrate was concentrated under reduced pressure and purified by silica gel column chromatography, eluting with petroleum ether/ethyl acetate (4:1), to afford 9-benzyl-9-azabicyclo[4.2.1]nonan-2-one (14 g, 84% yield). LCMS (ES, m/z): RT=1.04 min, m/z=230.1[M+H]*.

Step 4: Into a 1 L round-bottom flask were added 9-benzyl-9-azabicyclo[4.2.1]nonan-2-one (14 g, 61.84 mmol, 1 equiv), Pd/C (6.00 g, 10% on carbon, wet), ditertbutyldicarbonate (Boc₂O) (7.45 g, 34.16 mmol, 0.55 equiv) in 300 mL of isopropanol at room temperature. The resulting mixture was stirred for 12 h at room temperature under hydrogen atmosphere. The reaction was monitored by LCMS, filtered, and the filter cake was washed with isopropanol (3×150 mL). The filtrate was concentrated under reduced pressure and purified by reversed-phase flash chromatography (C18 silica gel; mobile phase, acetonitrile in water, 10% to 50% gradient in 10 min; detector, UV 220 nm) to provide tert-butyl 2-oxo-9-azabicyclo[4.2.1]nonane-9-carboxylate (7.3 g, 50% yield). LCMS (ES, m/z): RT=0.79 min, m/z=240.2[M+H]⁺.

Step 5: To a stirred solution of tert-butyl 2-oxo-9-azabicyclo[4.2.1]nonane-9-carboxylate (7.3 g, 30.41 mmol, 1 equiv) in tetrahydrofuran (THF) (240 mL) was added lithium diisopropylamide (LDA) (40 mL, 80 mmol, 2.5 equiv, 2.0 M in tetrahydrofuran (THF)) at −78° C. under nitrogen atmosphere. The resulting mixture was stirred for 1 h at −78° C. under nitrogen atmosphere. To the above resulting solution was added tert-butyldimethylsilyl trifluoromethanesulfonate (TBSOTf) (32.5 g, 45.1 mmol, 1.5 equiv). The resulting mixture was stirred for 1 h at room temperature under nitrogen atmosphere. The reaction was monitored by LCMS, quenched with water (200 mL) and the mixture was extracted with ethyl acetate (EtOAc) (3×100 mL). The combined organic layers were dried over anhydrous Na₂SO₄, filtered the filtrate was concentrated under reduced pressure. The residue was purified by reversed-phase flash chromatography (C18 silica gel; mobile phase, acetonitrile in water, 10% to 50% gradient in 10 min; detector, UV 254 nm) to provide tert-butyl 2-((tert-butyldimethylsilyl)oxy)-9-azabicyclo[4.2.1]non-2-ene-9-carboxylate (5.80 g, 54% yield). LCMS (ES, m/z): RT=2.48 min, m/z=354.2[M+H]⁺.

Step 6: To a stirred solution of tert-butyl 2-[(tert-butyldimethylsilyl)oxy]-9-azabicyclo[4.2.1]non-2-ene-9-carboxylate (5.80 g, 16.23 mmol, 1 equiv) in dimethylsulfoxide (100 mL) was added palladium acetate (Pd(OAc)₂) (10.66 g, 48.70 mmol, 3 equiv). The resulting mixture was stirred for 12 h at 40° C. under oxygen atmosphere. The reaction was monitored by LCMS, quenched with water (1 L) and the mixture was extracted with ethyl acetate (EtOAc) (3×300 mL). The combined organic layers were concentrated under vacuum and the residue was purified by reversed-phase flash chromatography (C18 silica gel; mobile phase, acetonitrile in water, 10% to 50% gradient in 10 min; detector, UV 254 nm) to provide tert-butyl 2-oxo-9-azabicyclo[4.2.1]non-3-ene-9-carboxylate (2.6 g, 68% yield). LCMS (ES, m/z): RT=0.84 min, m/z=238.1[M+H]⁺.

Step 7: To a stirred solution of tert-butyl 2-oxo-9-azabicyclo[4.2.1]non-3-ene-9-carboxylate (2.6 g, 10.94 mmol, 1 equiv) in tetrahydrofuran (THF) (80 mL) was added dimethylcopperlithium (30 mL, 16.41 mmol, 1.5 equiv, 0.5M in ether) at −78° C. under nitrogen atmosphere. The reaction was stirred for 1h at −78° C. under nitrogen atmosphere, monitored by LCMS and was quenched with sat. NH₄Cl (aq.) at 0° C. The mixture was extracted with ethyl acetate (EtOAc) (3×200 mL) and the combined organic layers were concentrated under vacuum. The residue was purified by reversed-phase flash chromatography (C18 silica gel; mobile phase, acetonitrile in water, 10% to 50% gradient in 10 min; detector, UV 254 nm) to provide tert-butyl 4-methyl-2-oxo-9-azabicyclo[4.2.1]nonane-9-carboxylate (2.2 g, 79% yield). LCMS (ES, m/z): RT=0.71 min, m/z=254.2[M+H]⁺.

Step 8: To a stirred solution of tert-butyl 4-methyl-2-oxo-9-azabicyclo[4.2.1]nonane-9-carboxylate (2.2 g, 8.62 mmol, 1 equiv) in methanol (80 mL) was added benzylamine (1.86 g, 17.24 mmol, 2 equiv) at room temperature. The resulting mixture was stirred for 12 h at 60° C. followed by the addition of NaBH₃CN (5.45 g, 86.20 mmol, 10 equiv). The reaction was then stirred for 2 h at 60° C., monitored by LCMS and concentrated under vacuum. The residue was purified by reversed-phase flash chromatography (C18 silica gel; mobile phase, acetonitrile in water, 10% to 50% gradient in 10 min; detector, UV 200 nm) to provide tert-butyl 2-(benzylamino)-4-methyl-9-azabicyclo[4.2.1]nonane-9-carboxylate (2.1 g, 70% yield). LCMS (ES, m/z): RT=1.19 min, m/z=345.2[M+H]⁺.

Step 9: To a stirred solution of tert-butyl 2-(benzylamino)-4-methyl-9-azabicyclo[4.2.1]nonane-9-carboxylate (2.1 g, 6.0 mmol, 1 equiv) in isopropyl alcohol (36 mL) was added Pd/C (1.20 g, 10% on carbon, wet) at room temperature. The resulting mixture was stirred for 12 h at room temperature under hydrogen atmosphere. The reaction was monitored by LCMS, filtered, and the filter cake was washed with isopropyl alcohol (3×20 mL). The residue was concentrated under reduced pressure and resulted in tert-butyl 2-amino-4-methyl-9-azabicyclo[4.2.1]nonane-9-carboxylate (amine (iv) reagent) (1.5 g, 97% yield). LCMS (ES, m/z): RT=0.75 min, m/z=255.2[M+H]⁺.

Step 10: To a stirred solution of 1-[4-(trifluoromethyl)phenyl]cyclopropane-1-carboxylic acid (carboxyl (ii) reagent) (1.20 g, 4.94 mmol, 1 equiv) and of the amine (iv) reagent of step 9 (1.5 g, 5.93 mmol, 1.2 equiv) in dimethylformamide (40 mL) was added hydroxybenzotriazole (HOBT) (818.34 mg, 5.93 mmol, 1.2 equiv), 1-ethyl-3-(3-dimethylaminopropyl)carbodiimide (EDCI) (2.13 g, 9.88 mmol, 2 equiv) and diisopropylethylamine (DIEA) (1.27 g, 9.88 mmol, 2 equiv) at room temperature. The resulting mixture was stirred for 1 h at room temperature. The reaction was monitored by LCMS, quenched with water (400 mL) at room temperature and the mixture was extracted with ethyl acetate (EtOAc) (3×200 mL). The combined organic layers were concentrated under vacuum and the residue was purified by reversed-phase flash chromatography (C18 silica gel; mobile phase, acetonitrile in water, 10% to 50% gradient in 10 min; detector, UV 200 nm) to provide tert-butyl 4-methyl-2-{1-[4-(trifluoromethyl)phenyl]cyclopropaneamido}-9-azabicyclo[4.2.1]nonane-9-carboxylate (1.80 g, 75% yield). LCMS (ES, m/z): RT=1.19 min, m/z=467.2[M+H]⁺.

Step 11: A solution of tert-butyl 4-methyl-2-{1-[4-(trifluoromethyl)phenyl]cyclopropaneamido}-9-azabicyclo[4.2.1]nonane-9-carboxylate (1.80 g, 3.86 mmol, 1 equiv) in 25 mL of anhydrous 4.0M HCl in 1,4-dioxane was stirred for 1 h at room temperature. The reaction was monitored by LCMS and concentrated under vacuum. This resulted in N-{4-methyl-9-azabicyclo[4.2.1]nonan-2-yl}-1-[4-(trifluoromethyl)phenyl]cyclopropane-1-carboxamide hydrochloride (1.60 g, 89% yield). LCMS (ES, m/z): RT=0.65 min, m/z=367.2[M+H]⁺.

Step 12: To a stirred solution of N-{4-methyl-9-azabicyclo[4.2.1]nonan-2-yl}-1-[4-(trifluoromethyl)phenyl]cyclopropane-1-carboxamide hydrochloride (1.60 g, 4.37 mmol, 1 equiv) in acetonitrile (45 mL) was added BrCN (4.77 g, 43.70 mmol, 10.00 equiv) and K₂CO₃ (1.80 mg, 13.11 mmol, 3.00 equiv) at room temperature. The resulting mixture was stirred for 1 h at 80° C., monitored by LCMS and quenched by the addition of water (60 mL) at room temperature. The mixture was extracted with ethyl acetate (EtOAc) (3×50 mL) and concentrated under vacuum. The residue was purified by Prep-TLC (petroleum ether/ethyl acetate 2:1) to afford N-{9-cyano-4-methyl-9-azabicyclo[4.2.1]nonan-2-yl}-1-[4-(trifluoromethyl)phenyl]cyclopropane-1-carboxamide (1.20 g, 71% yield). LCMS (ES, m/z): RT=1.02 min, m/z=392.2[M+H]*.

Step 13: To a stirred solution of N-{9-cyano-4-methyl-9-azabicyclo[4.2.1]nonan-2-yl}-1-[4-(trifluoromethyl)phenyl]cyclopropane-1-carboxamide (1.20 g, 3.06 mmol, 1 equiv) and dibutyltin oxide (2.30 g, 9.18 mmol, 3 equiv) in 1,4-dioxane (30 mL) was added trimethylsilyl azide (TMSN₃) (2.10 g, 9.18 mmol, 3 equiv) at room temperature. The resulting mixture was stirred for 1 h at 100° C., monitored by LCMS and quenched with water (50 mL) at room temperature. The mixture was extracted with ethyl acetate (EtOAc) (3×50 mL) and the combined organic layers were concentrated under vacuum. The residue was purified by Prep-HPLC (XBridge Prep Phenyl OBD Column19*250 mm, 5 μm; Mobile Phase A: water (0.05% trifluoroacetic acid), Mobile Phase B: acetonitrile; Flow rate: 25 mL/min; Gradient: 33% B to 43% B in 10 min; Wave Length: 254/220 nm) to afford as the first eluting peak assumed Compound 104″″ (quad prime)* as a mixture of two stereoisomers (220 mg, 17% yield, RT(min): 13.06); as the second eluting peak a mixture of assumed Compound 104′ (prime)* and assumed Compound 104′″ (triple prime)* as a mixture of four stereoisomers (420.00 mg, 32% yield, RT(min): 19.12), and as the third eluting peak assumed Compound 104″ (double prime)* as a mixture of two stereoisomers (70 mg, 5% yield, RT(min): 20.00). Assumed Compound 104″″ (quad prime)*: LCMS (ES, m/z): RT=1.31 min, m/z=435.2[M+H]⁺. Assumed mixture of Compound 104′ (prime)* and Compound 104′″ (triple prime)*: LCMS (ES, m/z): RT=1.52 min, m/z=435.2[M+H]⁺. Assumed Compound 104*″ (double prime): LCMS (ES, m/z): RT=1.72 min, m/z=435.2[M+H]⁺.

Step 14: The assumed Compound 104″″ (quad prime)* mixture of two stereoisomers (30 mg, 96% purity) was purified by Prep-Chiral-HPLC (CHIRALPAK ID, 2*25 cm, 5 μm; Mobile Phase A: Hexanes (0.1% TFA), Mobile Phase B: (EtOH:DCM=1:1); Flow rate: 20 mL/min; Gradient: isocratic 10; Wave Length: 220/254 nm) to provide Compound 104G* (8.00 mg, 21% yield, RT(min): 18.15) as the first eluting peak, and Compound 104H* (7.20 mg, 21% yield, RT(min): 23.19) as the second eluting peak. Stereochemistry arbitrarily assigned.

Compound 104G*: LCMS: (ES, m/z): RT=1.45 min, m/z=435.2[M+H]⁺; ¹H NMR (400 MHz, Methanol-d₄) δ 7.64-7.36 (m, 4H), 4.49-4.38 (m, 1H), 4.12-4.03 (m, 1H), 3.99 (q, J=3.1 Hz, 1H), 2.43-2.31 (m, 1H), 2.31-2.21 (m, 1H), 2.18-2.08 (m, 1H), 1.84-1.73 (m, 2H), 1.71-1.57 (m, 3H), 1.57-1.45 (m, 1H), 1.30-1.22 (m, 1H), 1.22-1.09 (m, 2H), 1.08-0.98 (m, 1H), 0.88 (d, J=6.4 Hz, 3H).

Compound 104H*: LCMS: (ES, m/z): RT=1.44 min, m/z=435.2[M+H]⁺; ¹H NMR (400 MHz,) δ 8.94 (d, J=8.2 Hz, 2H), 8.88 (d, J=8.1 Hz, 2H), 5.86 (d, J=8.8 Hz, 1H), 5.48 (t, J=9.0 Hz, 1H), 5.42-5.33 (m, 1H), 3.84-3.72 (m, 1H), 3.71-3.61 (m, 1H), 3.59-3.45 (m, 1H), 3.26-3.13 (m, 2H), 3.12-2.98 (m, 3H), 2.97-2.86 (m, 1H), 2.69-2.51 (m, 3H), 2.48-2.39 (m, 1H), 2.28 (d, J=6.2 Hz, 3H).

Step 15: The mixture of assumed Compound 104′ (prime)* and assumed Compound 104′″ (triple prime)* as a mixture of four stereoisomers (170 mg, 99.5% purity) was purified by Prep-Chiral-HPLC (CHIRALPAK SB 2*25 cm, 5 μm; Mobile Phase A: Hexanes (0.1% TFA), Mobile Phase B: (EtOH:DCM=1:1); Flow rate: 20 mL/min; Gradient: isocratic 10; Wave Length: 220/254 nm; RT3(min): 11.72) provided as the first eluting peak the assumed Compound 104′″ (triple prime)* as a mixture of two stereoisomers (45 mg, 9% yield, RT(min): 8.12), Compound 104A* (33.80 mg, 7% yield, RT(min): 10.24) as the second eluting peak, and Compound 104B* (34.30 mg, 7% yield) as the third eluting peak. Stereochemistry arbitrarily assigned.

Compound 104A*: LCMS: (ES, m/z): RT=0.88 min, m/z=435.2[M+H]⁺; ¹H NMR (400 MHz, Methanol-d₄) δ 7.72 (d, J=8.2 Hz, 2H), 7.62 (d, J=8.1 Hz, 2H), 4.55-4.43 (m, 2H), 4.36-4.30 (m, 1H), 2.22-1.99 (m, 2H), 1.97-1.78 (m, 2H), 1.78-1.68 (m, 1H), 1.66-1.48 (m, 4H), 1.44-1.35 (m, 2H), 1.30-1.22 (m, 1H), 1.22-1.11 (m, 1H), 0.81 (d, J=6.6 Hz, 3H).

Compound 104B*: LCMS: (ES, m/z): RT=0.88 min, m/z=435.2[M+H]+; ¹H NMR (400 MHz, Methanol-d₄) δ 7.76-7.69 (m, 2H), 7.66-7.58 (m, 2H), 4.56-4.42 (m, 2H), 4.37-4.29 (m, 1H), 2.21-2.01 (m, 2H), 1.96-1.80 (m, 2H), 1.78-1.69 (m, 1H), 1.66-1.49 (m, 4H), 1.44-1.35 (m, 2H), 1.29-1.22 (m, 1H), 1.21-1.13 (m, 1H), 0.81 (d, J=6.6 Hz, 3H).

Step 16: The assumed Compound 104′″ (triple prime)* mixture of two stereoisomers from step 15 (45 mg, 95% purity) was purified by chiral-Prep-HPLC (CHIRALPAK IH, 2*25 cm, 5 μm; Mobile Phase A: Hexanes (0.1% TFA), Mobile Phase B: (EtOH:DCM=1:1); Flow rate: 20 mL/min; Gradient: isocratic 7) provided Compound 104E* (7.80 mg, 14% yield, RT(min): 13.79) as the first eluting peak, and Compound 104F* (11.50 mg, 20% yield, RT(min): 18.25) as the second eluting peak. Stereochemistry arbitrarily assigned.

Compound 104E*: LCMS: (ES, m/z): RT=0.86 min, m/z=435.2[M+H]⁺; ¹H NMR (400 MHz, Methanol-d₄) δ 7.72 (d, J=8.1 Hz, 2H), 7.63 (d, J=8.1 Hz, 2H), 4.22-3.99 (m, 3H), 2.61-2.49 (m, 1H), 2.21-2.08 (m, 1H), 2.03-1.91 (m, 1H), 1.90-1.76 (m, 2H), 1.69-1.57 (m, 3H), 1.54 (d, 1H), 1.48-1.35 (m, 1H), 1.21-1.09 (m, 2H), 0.89 (d, J=6.6 Hz, 3H), 0.78-0.60 (m, 1H).

Compound 104F*: LCMS: (ES, m/z): RT=0.87 min, m/z=435.2[M+H]⁺; ¹H NMR (400 MHz, Methanol-d₄) δ 7.72 (d, J=8.1 Hz, 2H), 7.63 (d, J=8.1 Hz, 2H), 4.21-4.10 (m, 2H), 4.10-4.03 (m, 1H), 2.62-2.48 (m, 1H), 2.21-2.07 (m, 1H), 2.03-1.91 (m, 1H), 1.90-1.74 (m, 2H), 1.69-1.57 (m, 3H), 1.57-1.50 (m, 1H), 1.47-1.36 (m, 1H), 1.23-1.10 (m, 2H), 0.89 (d, J=6.7 Hz, 3H), 0.69 (t, J=14.0, 11.0 Hz, 1H).

Step 17: The assumed Compound 104″ (double prime)* mixture of two stereoisomers (70 mg, 95% purity) was purified by Prep-Chiral-HPLC (CHIRAL ART Cellulose-SB, 2*25 cm, 5 μm; Mobile Phase A: Hexanes (0.1% TFA), Mobile Phase B: EtOH:DCM=1:1; Flow rate: 20 mL/min; Gradient: 10% B to 10% B in 16 min; Wave Length: 220/254 nm) to provide Compound 104C* (27.10 mg, 2% yield, RT(min): 9.11) as the first eluting peak, and Compound 104D* (24.80 mg, 2% yield, RT(min): 13.45) as the second eluting peak. Stereochemistry arbitrarily assigned.

Compound 104C*: LCMS104: (ES, m/z): RT=0.81 min, m/z=435.2[M+H]⁺; ¹H NMR (400 MHz, Methanol-d₄) δ 7.71 (d, J=8.2 Hz, 2H), 7.66-7.55 (m, 2H), 4.32-4.20 (m, 2H), 4.14-4.06 (m, 1H), 2.54-2.35 (m, 2H), 2.00-1.87 (m, 1H), 1.84-1.69 (m, 2H), 1.64-1.50 (m, 3H), 1.49-1.27 (m, 3H), 1.26-1.13 (m, 3H), 0.91 (d, J=6.7 Hz, 3H).

Compound 104D*: LCMS: (ES, m/z): RT=1.46 min, m/z=435.2[M+H]⁺; ¹H NMR (400 MHz, Methanol-d₄) δ 7.71 (d, J=8.2 Hz, 2H), 7.61 (d, J=8.2 Hz, 2H), 6.69 (d, J=8.0 Hz, 1H), 4.32-4.19 (m, 2H), 4.16-4.07 (m, 1H), 2.54-2.36 (m, 2H), 2.00-1.87 (m, 1H), 1.85-1.69 (m, 2H), 1.65-1.50 (m, 3H), 1.50-1.41 (m, 1H), 1.32 (d, J=6.5 Hz, 1H), 1.27-1.11 (m, 3H), 0.91 (d, J=6.6 Hz, 3H).

Compounds provided in the below Table A were prepared, or may be prepared, following the General Procedures and Examples as described above. LC-MS data is provided for each compound prepared according to the described Procedure. Dashed (--) line in Table A signifies no data obtained. For purposes of the Examples, including the data provided in the Assay Methods section, “Rac-X” signifies a mixture of 2 or more stereoisomers, e.g., Compounds X′ X″, X″, X″″, XA, XB, XC, XD, XE, XF, XG, and/or XH.

TABLE A
Additional Compounds

| | | LCMS | |
|---|---|---|---|
| # | Compound | (m/z) | Procedure |
| Rac-37 | 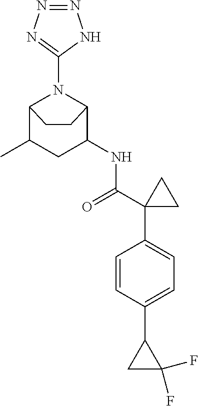 [SMILES: CC1CC(NC(=O)C2(c3ccc(C4CC4(F)F)cc3)CC2)C2CCC1N2c1nnn[nH]1] | 429.20 | Rac-37, 37′*, 37″*, 37A*, 37B*, 37C* and 37D* were prepared according to Protocol A and Example 35 using 4-methyl-8-(1H-tetrazol- 5-yl)-8-azabicyclo[3.2.1]octan-2-amine as the amine (i) reagent and 1-[4-(2,2- difluorocyclopropyl)phenyl]cyclopropane-1- carboxylic acid as the carboxyl (ii) reagent. Rac-37 was then subjected to reverse phase column chromatography (Xselect CSH OBD Column 30*150 mm, 5 umn; Mobile Phase A: Water (0.05% trifluoroacetic acid), Mobile Phase B: acetonitrile; Flow rate: 60 mL/min; Gradient: 5% B to 5% B in 2 min, 30% B to 40% B in 8 min, 40% B to 50% B in 14 min; Wave Length: 254 nm/220 nm) to afford a mixture of compounds 37B* and 37C* as the first eluting peak, and a mixture of compounds 37A* and 37D* as the second eluting peak. Chiral separation conditions for assumed trans isomers, 37B* and 37C*: Column: CHIRALPAK ID, 2*25 cm, 5 μm; Mobile Phase A: Hexanes (0.1% trifluoroacetic acid), Mobile Phase B: Ethanol:dichloromethane = 1:1; Flow rate: 20 mL/min; Gradient: 30% B to 30% B in 7 min; Wave Length: 220/254 nm. |
| | 4-methyl-8-(1H-tetrazol-5-yl)-8- | | Chiral separation conditions for assumed cis |
| | azabicyclo[3.2.1]octan-2-yl)-1-(4-(2,2- | | isomers, 37A* and 37D*: Column: CHIRAL |
| | difluorocyclopropyl)phenyl)cyclopropane-1- | | ART Cellulose-SC, 2*25 cm, 5 μm; Mobile |
| | carboxamide | | Phase A: Hexanes (0.1% trifluoroacetic acid), |
| 37′* | N-((1R,2R,5S)-4-methyl-8-(1H-tetrazol-5-yl)-8- | 429.20 | Mobile Phase B: Ethanol:dichloromethane = 1:1; |
| | azabicyclo[3.2.1]octan-2-yl)-1-(4-(2,2- | | Flow rate: 20 mL/min; Gradient: 30% B to 30% |
| | difluorocyclopropyl)phenyl)cyclopropane-1- | | B in 11.5 min; Wave Length: 220/254 nm. |
| | carboxamide | | |
| 37″* | N-((1S,2S,5R)-4-methyl-8-(1H-tetrazol-5-yl)-8- | 429.20 | |
| | azabicyclo[3.2.1]octan-2-yl)-1-(4-(2,2- | | |
| | difluorocyclopropyl)phenyl)cyclopropane-1- | | |
| | carboxamide | | |
| 37A* | N-((1R,2R,4R,5S)-4-methy1-8-(1H-tetrazol-5- | 429.20 | Compound 37A*: Chiral RT(min): 7.28 |
| | yl)-8-azabicyclo[3.2.1]octan-2-yl)-1-(4-(2,2- | | |
| | difluorocyclopropyl)phenyl)cyclopropane-1- | | |
| | carboxamide | | |
| 37B* | N-((1R,2S,4R,5S)-4-methyl-8-(1H-tetrazol-5- | 429.20 | Compound 37B*: Chiral RT(min): 4.55 |
| | yl)-8-azabicyclo[3.2.1]octan-2-yl)-1-(4-(2,2- | | |
| | difluorocyclopropyl)phenyl)cyclopropane-1- | | |
| | carboxamide | | |
| 37C* | N-((1S,2R,4S,5R)-4-methyl-8-(1H-tetrazol-5- | 429.20 | Compound 37C*: Chiral RT(min): 5.71 |
| | yl)-8-azabicyclo[3.2.1]octan-2-yl)-1-(4-(2,2- | | |
| | difluorocyclopropyl)phenyl)cyclopropane-1- | | |
| | carboxamide | | |
| 37D* | N-((1S,2S,4S,5R)-4-methy1-8-(1H-tetrazol-5- | 429.20 | Compound 37D*: Chiral RT(min): 9.25 |
| | yl)-8-azabicyclo[3.2.1]octan-2-yl)-1-(4-(2,2- | | |
| | difluorocyclopropyl)phenyl)cyclopropane-1- | | |
| | carboxamide | | |
| 38A | (R)-N-(1-(1H-tetrazol-5-yl)piperidin-3-yl)-1-(p- | 327.2 | Compound 38A was prepared according to |
| | tolyl)cyclopropane-1-carboxamide | | Protocol A and Example 1 using (R)-1-(1H- |
| | | | tetrazol-5-yl)piperidin-3-amine as the amine (i) |
| | | | reagent and 1-(p-tolyl)cyclopropane-1- |
| | | | carboxylic acid--methane as the carboxyl (ii) |
| | | | reagent. |
| 38B | (S)-N-(1-(1H-tetrazol-5-yl)piperidin-3-yl)-1-(p- | — | Compound 38B may prepared according to |
| | tolyl)cyclopropane-1-carboxamide | | Protocol A and Example 1 using (S)-1-(1H- |
| | | | tetrazol-5-yl)piperidin-3-amine as the amine (i) |
| | | | reagent and 1-(p-tolyl)cyclopropane-1- |
| | | | carboxylic acid-- methane as the carboxyl (ii) |
| | | | reagent. |
| 39A | 1-(4-bromophenyl)-N-[(3R)-1-(1H-tetrazol-5- | 391.1 | Compound 39A was prepared according to |
| | yl)-3-piperidyl]cyclopropanecarboxamide | | Protocol A and Example 1 using (R)-1-(1H- |
| | | | tetrazol-5-yl)piperidin-3-amine as the amine (i) |
| | | | reagent and 1-(4-bromophenyl)cyclopropane-1- |
| | | | carboxylic acid as the carboxyl (ii) reagent. |
| 39B | 1-(4-bromophenyl)-N-[(3S)-1-(1H-tetrazol-5- | — | Compound 39B may be prepared according to |
| | yl)-3-piperidyl]cyclopropanecarboxamide | | Protocol A and Example 1 using (S)-1-(1H- |
| | | | tetrazol-5-yl)piperidin-3-amine as the amine (i) |
| | | | reagent and 1-(4-bromophenyl)cyclopropane-1- |
| | | | carboxylic acid as the carboxyl (ii) reagent. |
| 40A | 1-(4-chlorophenyl)-N-[(2R,3R)-2-methyl-1- | 361.2 | Compound 40A was prepared according to |
| | (1H-tetrazol-5-yl)-3- | | Protocol C and Example 9 using tert-butyl |
| | piperidyl]cyclopropanecarboxamide | | (2R,3R)-3-amino-2-methylpiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 40B | 1-(4-chlorophenyl)-N-[(2R,3S)-2-methyl-1-(1H- | — | Compound 40B may be prepared according to |
| | tetrazol-5-yl)-3- | | Protocol C and Example 9 tert-butyl (2R,3S)-3- |
| | piperidyl]cyclopropanecarboxamide | | amino-2-methylpiperidine-1-carboxylate as the |
| | | | protected amine (iv) reagent and 1-(4- |
| | | | chlorophenyl)cyclopropane-1-carboxylic acid as |
| | | | the carboxyl (ii) reagent. |
| 40C | 1-(4-chlorophenyl)-N-[(2S,3R)-2-methyl-1-(1H- | 361.2 | Compound 40C was prepared according to |
| | tetrazol-5-yl)-3- | | Protocol C and Example 9 using tert-butyl |
| | piperidyl]cyclopropanecarboxamide | | (2S,3R)-3-amino-2-methylpiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 40D | 1-(4-chlorophenyl)-N-[(2S,3S)-2-methyl-1-(1H- | — | Compound 40D may be prepared according to |
| | tetrazol-5-yl)-3- | | Protocol C and Example 9 using tert-butyl |
| | piperidyl]cyclopropanecarboxamide | | (2S,3S)-3-amino-2-methylpiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 41A | 1-(4-chlorophenyl)-N-[(3R,6S)-6-methyl-1-(1H- | — | Compound 41A may be prepared according to |
| | tetrazol-5-yl)-3- | | Protocol C and Example 9 using tert-butyl |
| | piperidyl]cyclopropanecarboxamide | | (2S,5R)-5-amino-2-methylpiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 41B | 1-(4-chlorophenyl)-N-[(3S,6S)-6-methyl-1-(1H- | — | Compound 41B may be prepared according to |
| | tetrazol-5-yl)-3- | | Protocol C and Example 9 using tert-butyl |
| | piperidyl]cyclopropanecarboxamide | | (2S,5S)-5-amino-2-methylpiperidine-1- |
| | | | carboxylate (iv) reagent and 1-(4- |
| | | | chlorophenyl)cyclopropane-1-carboxylic acid as |
| | | | the carboxyl (ii) reagent. |
| 41C | 1-(4-chlorophenyl)-N-[(3R,6R)-6-methyl-1- | 361.0 | Compound 41C was prepared according to |
| | (1H-tetrazol-5-yl)-3- | | Protocol C and Example 9 using tert-butyl |
| | piperidyl]cyclopropanecarboxamide | | (2R,5R)-5-amino-2-methylpiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 41D | 1-(4-chloropheny1)-N-[(3S,6R)-6-methyl-1-(1H- | — | Compound 41D may be prepared according to |
| | tetrazol-5-yl)-3- | | Protocol C and Example 9 using tert-butyl |
| | piperidyl]cyclopropanecarboxamide | | (2R,5S)-5-amino-2-methylpiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| Rac-42 | 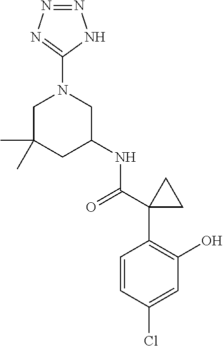 [SMILES: CC1(C)CC(NC(=O)C2(c3ccc(Cl)cc3O)CC2)CN(c2nnn[nH]2)C1] | 391.1 | Rac-42, 42A* and 40B* were prepared according to Protocol C and Example 9 using tert-butyl 5-amino-3,3-dimethylpiperidine-1- carboxylate as the protected amine (iv) reagent and 1-(4-chloro-2- methoxyphenyl)cyclopropane-1-carboxylic acid as the carboxyl (ii) reagent. chiral HPLC using the following conditions: CHIRAL ART Amylose-SA, 2*25 cm, 5 μm; Mobile Phase A: Hexanes (0.1% trifluoroacetic acid), Mobile Phase B: ethanol; Flow rate: 20 mL/min; Gradient: 5% B to 5% B in 26 min; Wave Length: 220/254 nm. |
| | 1-(4-chloro-2-hydroxyphenyl)-N-(5,5-dimethyl- | | |
| | 1-(1H-tetrazol-5-yl)piperidin-3- | | |
| | yl)cyclopropane-1-carboxamide | | |
| 42A* | (R)-1-(4-chloro-2-hydroxyphenyl)-N-(5,5- | 391.1 | Compound 42A*: Chiral RT(min): 21.90 |
| | dimethy1-1-(1H-tetrazol-5-yl)piperidin-3- | | |
| | yl)cyclopropane-1-carboxamide | | |
| 42B* | (S)-1-(4-chloro-2-hydroxyphenyl)-N-(5,5- | 391.1 | Compound 42B*: Chiral RT(min): 16.43 |
| | dimethyl-1-(1H-tetrazol-5-yl)piperidin-3- | | |
| | yl)cyclopropane-1-carboxamide | | |
| Rac-43 | 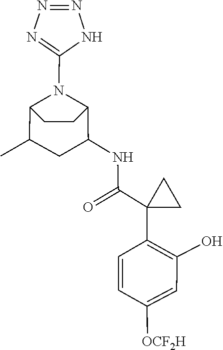 | 435.10 | Rac-43, 43′*, 43″*, 43A*, 43B*, 43C* and 43D* were prepared according to Protocol A and Example 35 using 4-methyl-8-(1H-tetrazol- 5-yl)-8-azabicyclo[3.2.1]octan-2-amine as the amine (i) reagent and 1-(4-(difluoromethoxy)-2- hydroxyphenyl)cyclopropane-1-carboxylic acid as the carboxyl (ii) reagent. Rac-43 was then subjected to reverse phase column chromatography (Column: Xselect CSH OBD Column 30*150 mm, 5 umn; Mobile Phase A: Water (0.05% trifluoroacetic acid), Mobile Phase B: ACETONITRILE; Flow rate: 60 mL/min mL/min; Gradient: 5% B to 5% B in 2 min, 24% B to 34% B in 8 min, 34% B to 44% B in 12 min; Wave Length: 254 nm/220 nm) to afford a mixture of compounds 43B* and 43C* as the first eluting peak, and a mixture of compounds 43A* and 43D* as the second eluting peak. Chiral separation conditions for assumed trans isomers, 43B* and 43C*: Column: CHIRALPAK ID, 2*25 cm, 5 μm; Mobile |
| | 4-methyl-8-(1H-tetrazol-5-yl)-8- | | Phase A: Hexanes (0.1% trifluoroacetic acid), |
| | azabicyclo[3.2.1]octan-2-yl)-1-(4- | | Mobile Phase B: Ethanol:dichloromethane = 1:1; |
| | (difluoromethoxy)-2- | | Flow rate: 20 mL/min; Gradient: 15% B to 15% |
| | hydroxyphenyl)cyclopropane-1-carboxamide | | B in 12.2 min; Wave Length: 220/254 nm. |
| 43′* | N-((1R,2R,5S)-4-methy1-8-(1H-tetrazol-5-yl)-8- | -- | Chiral separation conditions for assumed cis |
| | azabicyclo[3.2.1]octan-2-yl)-1-(4- | 435.10 | isomers, 43A* and 43D *: Column: Lux 5 um |
| | (difluoromethoxy)-2- | | Cellulose-4, 2.12*25 cm, 5 μm; Mobile Phase |
| | hydroxyphenyl)cyclopropane-1-carboxamide | | A: Hexanes (0.1% trifluoroacetic acid), Mobile |
| 43″* | N-((1S,2S,5R)-4-methy1-8-(1H-tetrazol-5-yl)-8- | 435.10 | Phase B: Ethanol:dichloromethane = 1:1; Flow |
| | azabicyclo[3.2.1]octan-2-yl)-1-(4- | | rate: 20 mL/min; Gradient: 10% B to 10% B in |
| | (difluoromethoxy)-2- | | 26 min; Wave Length: 220/254 nm. |
| | hydroxyphenyl)cyclopropane-1-carboxamide | | |
| 43A* | N-((1R,2R,4R,5S)-4-methyl-8-(1H-tetrazol-5- | 435.10 | Compound 43A*: Chiral RT(min): 18.0 |
| | yl)-8-azabicyclo[3.2.1]octan-2-yl)-1-(4- | | |
| | (difluoromethoxy)-2- | | |
| | hydroxyphenyl)cyclopropane-1-carboxamide | | |
| 43B* | N-((1R,2S,4R,5S)-4-methy1-8-(1H-tetrazol-5- | 435.10 | Compound 43B*: Chiral RT(min): 7.61 |
| | yl)-8-azabicyclo[3.2.1]octan-2-yl)-1-(4- | | |
| | (difluoromethoxy)-2- | | |
| | hydroxyphenyl)cyclopropane-1-carboxamide | | |
| 43C* | N-((1S,2R,4S,5R)-4-methy1-8-(1H-tetrazol-5- | 435.10 | Compound 43C*: Chiral RT(min): 9.93 |
| | yl)-8-azabicyclo[3.2.1]octan-2-yl)-1-(4- | | |
| | (difluoromethoxy)-2- | | |
| | hydroxyphenyl)cyclopropane-1-carboxamide | | |
| 43D* | N-((1S,2S,4S,5R)-4-methy1-8-(1H-tetrazol-5- | 435.10 | Compound 43D*: Chiral RT(min): 23.6 |
| | yl)-8-azabicyclo[3.2.1]octan-2-yl)-1-(4- | | |
| | (difluoromethoxy)-2- | | |
| | hydroxyphenyl)cyclopropane-1-carboxamide | | |
| 44A | 1-(4-chlorophenyl)-N-[(3R,5S)-5-fluoro-1-(1H- | 365.1 | Compound 44A was prepared according to |
| | tetrazol-5-yl)-3- | | Protocol C and Example 9 using tert-butyl |
| | piperidyl]cyclopropanecarboxamide | | (3R,5S)-3-amino-5-fluoropiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 44B | 1-(4-chlorophenyl)-N-[(3S,5S)-5-fluoro-1-(1H- | — | Compound 44B may be prepared according to |
| | tetrazol-5-yl)-3- | | Protocol C and Example 9 using tert-butyl |
| | piperidyl]cyclopropanecarboxamide | | (3S,5S)-3-amino-5-fluoropiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 44C | 1-(4-chlorophenyl)-N-[(3R,5R)-5-fluoro-1-(1H- | 365.1 | Compound 44C was prepared according to |
| | tetrazol-5-yl)-3- | | Protocol C and Example 9 using tert-butyl |
| | piperidyl]cyclopropanecarboxamide | | (3R,5R)-3-amino-5-fluoropiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 44D | 1-(4-chlorophenyl)-N-[(3S,5R)-5-fluoro-1-(1H- | — | Compound 44D may be prepared according to |
| | tetrazol-5-yl)-3- | | Protocol C and Example 9 using tert-butyl |
| | piperidyl]cyclopropanecarboxamide | | (3S,5R)-3-amino-5-fluoropiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 45A | 1-(4-chlorophenyl)-N-[(3R,5S)-1-(1H-tetrazol- | 414.9 | Compound 45A was prepared according to |
| | 5-yl)-5-(trifluoromethyl)-3- | | Protocol C and Example 9 using tert-butyl |
| | piperidyl]cyclopropanecarboxamide | | (3R,5S)-3-amino-5-(trifluoromethyl)piperidine- |
| | | | 1-carboxylate as the protected amine (iv) |
| | | | reagent and 1-(4-chlorophenyl)cyclopropane-1- |
| | | | carboxylic acid as the carboxyl (ii) reagent. |
| 45B | 1-(4-chlorophenyl)-N-[(3S,5S)-1-(1H-tetrazol- | — | Compound 45B may be prepared according to |
| | 5-yl)-5-(trifluoromethyl)-3- | | Protocol C and Example 9 using tert-butyl |
| | piperidyl]cyclopropanecarboxamide | | (3S,5S)-3-amino-5-(trifluoromethyl)piperidine- |
| | | | 1-carboxylate as the protected amine (iv) |
| | | | reagent and 1-(4-chlorophenyl)cyclopropane-1- |
| | | | carboxylic acid as the carboxyl (ii) reagent. |
| 45C | 1-(4-chlorophenyl)-N-[(3R,5R)-1-(1H-tetrazol- | — | Compound 45C may be prepared according to |
| | 5-yl)-5-(trifluoromethyl)-3- | | Protocol C and Example 9 using tert-butyl |
| | piperidyl]cyclopropanecarboxamide | | (3R,5R)-3-amino-5-(trifluoromethyl)piperidine- |
| | | | 1-carboxylate as the protected amine (iv) |
| | | | reagent and 1-(4-chlorophenyl)cyclopropane-1- |
| | | | carboxylic acid as the carboxyl (ii) reagent. |
| 45D | 1-(4-chlorophenyl)-N-[(3S,5R)-1-(1H-tetrazol- | — | Compound 45D may be prepared according to |
| | 5-yl)-5-(trifluoromethyl)-3- | | Protocol C and Example 9 using tert-butyl |
| | piperidyl]cyclopropanecarboxamide | | (3S,5R)-3-amino-5-(trifluoromethyl)piperidine- |
| | | | 1-carboxylate as the protected amine (iv) |
| | | | reagent and 1-(4-chlorophenyl)cyclopropane-1- |
| | | | carboxylic acid as the carboxyl (ii) reagent. |
| 46A | 1-(4-chlorophenyl)-N-((3R,5S)-5-methyl-1-(1H- | 361.3 | Compound 46A was prepared according to |
| | tetrazol-5-yl)piperidin-3-yl)cyclopropane-1- | | Protocol C and Example 9 using tert-butyl |
| | carboxamide | | (3R,5S)-3-amino-5-methylpiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 46B | 1-(4-chlorophenyl)-N-((3S,5S)-5-methyl-1-(1H- | — | Compound 46B may be prepared according to |
| | tetrazol-5-yl)piperidin-3-yl)cyclopropane-1- | | Protocol C and Example 9 using tert-butyl |
| | carboxamide | | (3S,5S)-3-amino-5-methylpiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 46C | 1-(4-chlorophenyl)-N-[(3R,5R)-5-methyl-1- | 361.3 | Compound 46C was prepared according to |
| | (1H-tetrazol-5-yl)-3- | | Protocol C and Example 9 using tert-butyl |
| | piperidyl]cyclopropanecarboxamide | | (3R,5R)-3-amino-5-methylpiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 46D | 1-(4-chlorophenyl)-N-[(3S,5R)-5-methyl-1-(1H- | — | Compound 46D may be prepared according to |
| | tetrazol-5-yl)-3- | | Protocol C and Example 9 using tert-butyl |
| | piperidyl]cyclopropanecarboxamide | | (3S,5R)-3-amino-5-methylpiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 47A | 1-(4-chloro-2-hydroxyphenyl)-N-((3R,5S)-5- | 377.1 | Compound 47A, and the corresponding |
| | methyl-1-(1H-tetrazol-5-yl)piperidin-3- | | protected —OMe phenol compound, were |
| | yl)cyclopropane-1-carboxamide | | prepared according to Protocol A and Example |
| | | | 1 using (3R,5S)-1-(1H-tetrazol-5-yl)-5- |
| | | | (methyl)piperidin-3-amine as the amine (i) |
| | | | reagent and 1-(2-methoxy-4- |
| | | | (chloro)phenyl)cyclopropane-1-carboxylic acid |
| | | | as the carboxyl (ii) reagent. |
| 47B | 1-(4-chloro-2-hydroxyphenyl)-N-((3S,5S)-5- | — | Compound 47B, and the corresponding |
| | methy1-1-(1H-tetrazol-5-yl)piperidin-3- | | protected -OMe phenol compound, may be |
| | yl)cyclopropane-1-carboxamide | | prepared according to Protocol A and Example |
| | | | 1 using (3S,5S)-1-(1H-tetrazol-5-yl)-5- |
| | | | (methyl)piperidin-3-amine as the amine (i) |
| | | | reagent and 1-(2-methoxy-4- |
| | | | (chloro)phenyl)cyclopropane-1-carboxylic acid |
| | | | as the carboxyl (ii) reagent. |
| 47C | 1-(4-chloro-2-hydroxyphenyl)-N-((3R,5R)-5- | — | Compound 47C, and the corresponding |
| | methyl-1-(1H-tetrazol-5-yl)piperidin-3- | | protected —OMe phenol compound, may be |
| | yl)cyclopropane-1-carboxamide | | prepared according to Protocol A and Example |
| | | | 1 using (3R,5R)-1-(1H-tetrazol-5-yl)-5- |
| | | | (methyl)piperidin-3-amine as the amine (i) |
| | | | reagent and 1-(2-methoxy-4- |
| | | | (chloro)phenyl)cyclopropane-1-carboxylic acid |
| | | | as the carboxyl (ii) reagent. |
| 47D | 1-(4-chloro-2-hydroxyphenyl)-N-((3S,5R)-5- | — | Compound 47D, and the corresponding |
| | methyl-1-(1H-tetrazol-5-yl)piperidin-3- | | protected —OMe phenol compound, may be |
| | yl)cyclopropane-1-carboxamide | | prepared according to Protocol A and Example |
| | | | 1 using (3S,5R)-1-(1H-tetrazol-5-yl)-5- |
| | | | (methyl)piperidin-3-amine as the amine (i) |
| | | | reagent and 1-(2-methoxy-4- |
| | | | (chloro)phenyl)cyclopropane-1-carboxylic acid |
| | | | as the carboxyl (ii) reagent. |
| 48A | 1-(4-(difluoromethyl)-2-hydroxyphenyl)-N- | 393.2 | Compound 48A, and the corresponding |
| | ((3R,5S)-5-methyl-1-(1H-tetrazol-5- | | protected —OMe phenol compound, was |
| | yl)piperidin-3-yl)cyclopropane-1-carboxamide | | prepared according to Protocol A and Example |
| | | | 1 using (3R,5S)-1-(1H-tetrazol-5-yl)-5- |
| | | | (methyl)piperidin-3-amine as the amine (i) |
| | | | reagent and 1-(2-methoxy-4- |
| | | | (difluoromethyl)phenyl)cyclopropane-1- |
| | | | carboxylic acid as the carboxyl (ii) reagent. |
| 48B | 1-(4-(difluoromethyl)-2-hydroxyphenyl)-N- | — | Compound 48B, and the corresponding |
| | ((3S,5S)-5-methyl-1-(1H-tetrazol-5- | | protected —OMe phenol compound, may be |
| | yl)piperidin-3-yl)cyclopropane-1-carboxamide | | prepared according to Protocol A and Example |
| | | | 1 using (3S,5S)-1-(1H-tetrazol-5-yl)-5- |
| | | | (methyl)piperidin-3-amine as the amine (i) |
| | | | reagent and 1-(2-methoxy-4- |
| | | | (difluoromethyl)phenyl)cyclopropane-1- |
| | | | carboxylic acid as the carboxyl (ii) reagent. |
| 48C | 1-(4-(difluoromethyl)-2-hydroxyphenyl)-N- | — | Compound 48C, and the corresponding |
| | ((3R,5R)-5-methyl-1-(1H-tetrazol-5- | | protected —OMe phenol compound, may be |
| | yl)piperidin-3-yl)cyclopropane-1-carboxamide | | prepared according to Protocol A and Example |
| | | | 1 using (3R,5R)-1-(1H-tetrazol-5-yl)-5- |
| | | | (methyl)piperidin-3-amine as the amine (i) |
| | | | reagent and 1-(2-methoxy-4- |
| | | | (difluoromethyl)phenyl)cyclopropane-1- |
| | | | carboxylic acid as the carboxyl (ii) reagent. |
| 48D | 1-(4-(difluoromethyl)-2-hydroxyphenyl)-N- | — | Compound 48D, and the corresponding |
| | ((3R,5S)-5-methyl-1-(1H-tetrazol-5- | | protected —OMe phenol compound, may be |
| | yl)piperidin-3-yl)cyclopropane-1-carboxamide | | prepared according to Protocol A and Example |
| | | | 1 using (3R,5S)-1-(1H-tetrazol-5-yl)-5- |
| | | | (methyl)piperidin-3-amine as the amine (i) |
| | | | reagent and 1-(2-methoxy-4- |
| | | | (difluoromethyl)phenyl)cyclopropane-1- |
| | | | carboxylic acid as the carboxyl (ii) reagent. |
| 49A | 1-(4-chlorophenyl)-N-[(3R,4R)-4-methy1-1- | 361.0 | Compound 49A was prepared according to |
| | (1H-tetrazol-5-yl)-3- | | Protocol C and Example 9 using tert-butyl |
| | piperidyl]cyclopropanecarboxamide | | (3R,4R)-3-amino-4-methylpiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 49B | 1-(4-chlorophenyl)-N-[(3S,4R)-4-methyl-1-(1H- | — | Compound 49B may be prepared according to |
| | tetrazol-5-yl)-3- | | Protocol C and Example 9 using tert-butyl |
| | piperidyl]cyclopropanecarboxamide | | (3S,4R)-3-amino-4-methylpiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 49C | 1-(4-chlorophenyl)-N-[(3R,4S)-4-methyl-1-(1H- | 361.0 | Compound 49C was prepared according to |
| | tetrazol-5-yl)-3- | | Protocol C and Example 9 using tert-butyl |
| | piperidyl]cyclopropanecarboxamide | | (3R,4S)-3-amino-4-methylpiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 49D | 1-(4-chlorophenyl)-N-[(3S,4S)-4-methyl-1-(1H- | — | Compound 49D may be prepared according to |
| | tetrazol-5-yl)-3- | | Protocol C and Example 9 using tert-butyl |
| | piperidyl]cyclopropanecarboxamide | | (3S,4S)-3-amino-4-methylpiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 50A | 1-(4-chlorophenyl)-N-[(3S,4R)-4-fluoro-1-(1H- | 365.0 | Compound 50A was prepared according to |
| | tetrazol-5-yl)-3- | | Protocol C and Example 9 using tert-butyl |
| | piperidyl]cyclopropanecarboxamide | | (3S,4R)-3-amino-4-fluoropiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 50B | 1-(4-chlorophenyl)-N-[(3R,4R)-4-fluoro-1-(1H- | | Compound 50B may be prepared according to |
| | tetrazol-5-yl)-3- | | Protocol C and Example 9 using tert-butyl |
| | piperidyl]cyclopropanecarboxamide | | (3R,4R)-3-amino-4-fluoropiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 50C | 1-(4-chlorophenyl)-N-[(3S,4S)-4-fluoro-1-(1H- | 365.0 | Compound 50C was prepared according to |
| | tetrazol-5-yl)-3- | | Protocol C and Example 9 using tert-butyl |
| | piperidyl]cyclopropanecarboxamide | | (3S,4S)-3-amino-4-fluoropiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 50D | 1-(4-chlorophenyl)-N-[(3R,4S)-4-fluoro-1-(1H- | | Compound 50D may be prepared according to |
| | tetrazol-5-yl)-3- | | Protocol C and Example 9 using tert-butyl |
| | piperidyl]cyclopropanecarboxamide | | (3R,4S)-3-amino-4-fluoropiperidine-1- |
| | | | carboxylate as the protected amine (iv) reagent |
| | | | and 1-(4-chlorophenyl)cyclopropane-1- |
| | | | carboxylic acid as the carboxyl (ii) reagent. |
| 51A | 1-(4-chlorophenyl)-N-[(3R)-1-(1H-tetrazol-5- | 363.0 | Compound 51A was prepared according to |
| | yl)azepan-3-yl]cyclopropanecarboxamide | | Protocol C and Example 9 using tert-butyl (R)- |
| | | | 3-aminoazepane-1-carboxylate as the protected |
| | | | amine (iv) reagent and 1-(4- |
| | | | chlorophenyl)cyclopropane-1-carboxylic acid as |
| | | | the carboxyl (ii) reagent. |
| 51B | 1-(4-chlorophenyl)-N-[(3S)-1-(1H-tetrazol-5- | — | Compound 51B may be prepared according to |
| | yl)azepan-3-yl]cyclopropanecarboxamide | | Protocol C and Example 9 using tert-butyl (S)- |
| | | | 3-aminoazepane-1-carboxylate as the protected |
| | | | amine (iv) reagent and 1-(4- |
| | | | chlorophenyl)cyclopropane-1-carboxylic acid as |
| | | | the carboxyl (ii) reagent. |
| 52A | (R)-N-(1-(1H-tetrazol-5-yl)azepan-3-yl)-1-(4- | 377.1 | Compound 52A, and the corresponding |
| | chloro-2-hydroxyphenyl)cyclopropane-1- | | protected —OMe phenol compound, were |
| | carboxamide | | prepared according to Protocol C and Example |
| | | | 9 using tert-butyl (R)-3-aminoazepane-1- |
| | | | carboxylate as the protected amine (iv) reagent |
| | | | and 1-(2-methoxy-4- |
| | | | (chloro)phenyl)cyclopropane-1-carboxylic acid |
| | | | as the carboxyl (ii) reagent. |
| 52B | (S)-N-(1-(1H-tetrazol-5-yl)azepan-3-yl)-1-(4- | — | Compound 52B, and the corresponding |
| | chloro-2-hydroxyphenyl)cyclopropane-1- | | protected —OMe phenol compound, may be |
| | carboxamide | | prepared according to Protocol C and Example |
| | | | 9 using tert-butyl (S)-3-aminoazepane-1- |
| | | | carboxylate as the protected amine (iv) reagent |
| | | | and 1-(2-methoxy-4- |
| | | | (chloro)phenyl)cyclopropane-1-carboxylic acid |
| | | | as the carboxyl (ii) reagent. |
| Rac-53 | 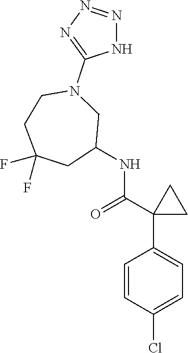 [SMILES: O=C(NC1CN(c2nnn[nH]2)CCC(F)(F)C1)C1(c2ccc(Cl)cc2)CC1] | 397.2 | Rac-53, 53A*, and 53B*, and the corresponding protected —OMe phenol compounds, were prepared according to Protocol C and Example 9 using tert-butyl 3-amino-5,5-difluoroazepane- 1-carboxylate as the protected amine (iv) reagent and 1-(4-chlorophenyl)cyclopropane-1- carboxylic acid as the carboxyl (ii) reagent. The racemic mixture was then subjected to prep chiral HPLC using the following conditions: CHIRAL ART Amylose-SA, 2*25 cm, 5 μm; Mobile Phase A: Hexanes (0.2% formic acid), Mobile Phase B: ethanol; Flow rate: 20 mL/min; Gradient: 10% B to 10% B in 21 min; Wave Length: 220/254 nm. |
| | 1-(4-chlorophenyl)-N-(5,5-difluoro-1-(1H- | | |
| | tetrazol-5-yl)azepan-3-yl)cyclopropane-1- | | |
| | carboxamide | | |
| 53A* | (R)-1-(4-chlorophenyl)-N-(5,5-difluoro-1-(1H- | 397.2 | Compound 53A*: Chiral RT(min): 16.40 |
| | tetrazol-5-yl)azepan-3-yl)cyclopropane-1- | | |
| | carboxamide | | |
| 53B* | (S)-1-(4-chlorophenyl)-N-(5,5-difluoro-1-(1H- | 397.2 | Compound 53B*: Chiral RT(min): 19.62 |
| | tetrazol-5-yl)azepan-3-yl)cyclopropane-1- | | |
| | carboxamide | | |
| Rac-54 | 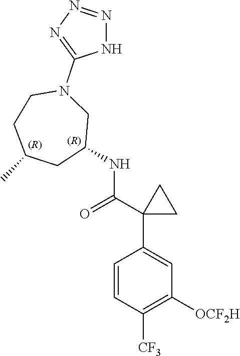 | 475.3 | Rac-54, 54A*, 54B*, 54C*, and 54D* were prepared according to Protocol A and Example 1 using (3R,5R)-5-methyl-1-(1H-tetrazol-5- yl)azepan-3-amine as the amine (i) reagent and 1-(3-(difluoromethoxy)-4- (trifluoromethyl)phenyl)cyclopropane-1- carboxylic acid as the carboxyl (ii) reagent. Rac-54 was then subjected to prep chiral HPLC using the following conditions: Column, XBridge Prep Phenyl OBD Column, 19*150 mm, 5 um; mobile phase, Water (10 mmol/L NH₄HCO₃) and acetonitrile (24% up to 330% in 10 min); Detector, UV 254 nm to provide a mixture of compounds 54B* and 54C* as the first eluting peak, and a mixture of compounds 54A* and 54D* as the second eluting peak. Chiral separation conditions for assumed cis isomers, 54A* and 54D *: Lux 5 um Cellulose-4, 2.12*25 cm, 5 um Mobile Phase A: Hexanes (0.1% trifluoroacetic acid), Mobile Phase B: isopropanol; Flow rate: 20 mL/min; Gradient: 15% B to 15% B in 18 min; Wave Length: |
| | 1-(3-(difluoromethoxy)-4- | | 254/220 nm. |
| | (trifluoromethyl)phenyl)-N-(5-methyl-1-(1H- | | Chiral separation conditions for assumed trans |
| | tetrazol-5-yl)azepan-3-yl)cyclopropane-1- | | isomers, 54B* and 54C *: Lux 5um Cellulose-4, |
| | carboxamide | | 2.12*25 cm, 5 um Mobile Phase A: Hexanes |
| | | | (0.1% trifluoroacetic acid), Mobile Phase B: |
| | | | isopropanol; Flow rate: 20 mL/min; Gradient: |
| | | | 30% B to 30% B in 10 min; Wave Length: |
| | | | 254/220 nm. |
| 54A* | 1-(3-(difluoromethoxy)-4- | 475.3 | Compound 54A*: Chiral RT(min): 17.36 |
| | (trifluoromethyl)phenyl)-N-((3R,5R)-5-methyl- | | |
| | 1-(1H-tetrazol-5-yl)azepan-3-yl)cyclopropane- | | |
| | 1-carboxamide | | |
| 54B* | 1-(3-(difluoromethoxy)-4- | 475.3 | Compound 54B*: Chiral RT(min): 7.49 |
| | (trifluoromethyl)phenyl)-N-((3S,5R)-5-methyl- | | |
| | 1-(1H-tetrazol-5-y1)azepan-3-yl)cyclopropane- | | |
| | 1-carboxamide | | |
| 54C* | 1-(3-(difluoromethoxy)-4- | 475.3 | Compound 54C*: Chiral RT(min): 9.80 |
| | (trifluoromethyl)phenyl)-N-((3R,5S)-5-methyl- | | |
| | 1-(1H-tetrazol-5-yl)azepan-3-y1)cyclopropane- | | |
| | 1-carboxamide | | |
| 54D* | 1-(3-(difluoromethoxy)-4- | 475.3 | Compound 54D*: Chiral RT(min): 13.28 |
| | (trifluoromethyl)phenyl)-N-((3S,5S)-5-methyl- | | |
| | 1-(1H-tetrazol-5-yl)azepan-3-yl)cyclopropane- | | |
| | 1-carboxamide | | |
| Rac-55 | 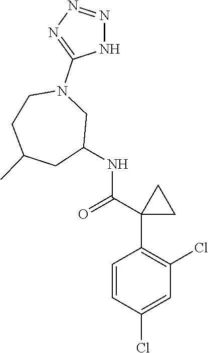 [SMILES: CC1CCN(c2nnn[nH]2)CC(NC(=O)C2(c3ccc(Cl)cc3Cl)CC2)C1] | 409.1 | Rac-55, 55A*, 55B*, 55C*, and 55D* were prepared according to Protocol A and Example 1 using (3R,5R)-5-methyl-1-(1H-tetrazol-5- yl)azepan-3-amine as the amine (i) reagent and 1-(2-chloro-4-chlorophenyl)cyclopropane-1- carboxylic acid as the carboxyl (ii) reagent. Rac-55 was then subjected to prep chiral HPLC using the following conditions: YMC-Actus Triart C18 ExRS, 30*150 mm, 5 μm; Mobile Phase A: Water (10 mmol/L NH₄HCO₃), Mobile Phase B: acetonitrile; Flow rate: 60 mL/min; Gradient: 16% B to 30% B in 10 min, 30% B; Wave Length: 254 nm to provide a mixture of compounds 55B* and 55C* as the first eluting peak, and a mixture of compounds 55A* and 55D* as the second eluting peak. Chiral separation conditions for assumed cis isomers, 55A* and 55D *: Lux 5 um Cellulose-4, 2.12*25 cm, 5 um; Mobile Phase A: Hexanes (0.1% trifluoroacetic acid), Mobile Phase B: isopropanol; Flow rate: 20 mL/min; Gradient: 30% B to 30% B in 18 min; Wave Length: |
| | 1-(3-(difluoromethoxy)-4- | | 254/220 nm. |
| | (trifluoromethyl)phenyl)-N-(5-methyl-1-(1H- | | Chiral separation conditions for assumed trans |
| | tetrazol-5-y1)azepan-3-yl)cyclopropane-1- | | isomers, 55B* and 55C *: Lux 5 um Cellulose-4, |
| | carboxamide | | 2.12*25 cm, 5 μm; Mobile Phase A: Hexanes |
| | | | (0.1% trifluoroacetic acid), Mobile Phase B: |
| | | | isopropanol; Flow rate: 20 mL/min; Gradient: |
| | | | 50% B to 50% B in 12 min; Wave Length: |
| | | | 254/220 nm. |
| 55A* | 1-(2,4-dichlorophenyl)-N-((3R,5R)-5-methyl-1- | 409.1 | Compound 55A*: Chiral RT(min): 16.70 |
| | (1H-tetrazol-5-yl)azepan-3-yl)cyclopropane-1- | | |
| | carboxamide | | |
| 55B* | 1-(2,4-dichlorophenyl)-N-((3S,5R)-5-methyl-1- | 409.1 | Compound 55B*: Chiral RT(min): 7.51 |
| | (1H-tetrazol-5-yl)azepan-3- | | |
| 55C* | 1-(2,4-dichlorophenyl)-N-((3R,5S)-5-methyl-1- | 409.1 | Compound 55C*: Chiral RT(min): 10.70 |
| | (1H-tetrazol-5-yl)azepan-3- | | |
| 55D* | 1-(2,4-dichlorophenyl)-N-((3S,5S)-5-methyl-1- | 409.1 | Compound 55D*: Chiral RT(min): 13.61 |
| | (1H-tetrazol-5-yl)azepan-3- | | |
| Rac-56 | 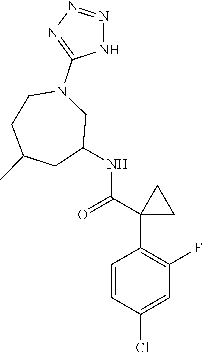 [SMILES: CC1CCN(c2nnn[nH]2)CC(NC(=O)C2(c3ccc(Cl)cc3F)CC2)C1] | 393.1 | Rac-56, 56A*, 56B*, 56C*, and 56D* was prepared according to Protocol A and Example 1 using 5-methyl-1-(1H-tetrazol-5-yl)azepan-3- amine as the amine (i) reagent and 1-(2-chloro- 4-chlorophenyl)cyclopropane-1-carboxylic acid as the carboxyl (ii) reagent. Rac-56 was then subjected to prep chiral HPLC using the following conditions: XBridge Shield RP18 OBD Column, 30*150 mm, 5 μm; Mobile Phase A: Water (10 mmol/L NH₄HCO₃), Mobile Phase B: acetonitrile; Flow rate: 60 mL/min; Gradient: 32% B to 36% B in 8 min; Wave Length: 254 nm to provide a mixture of compounds 56A* and 56D* as the first eluting peak, and a mixture of compounds 56B* and 56C* as the second eluting peak. Chiral separation conditions for assumed cis isomers, 56A* and 56D *: CHIRALPAK IG, 2*25 cm, 5 μm; Mobile Phase A: Hexanes (0.1% trifluoroacetic acid), Mobile Phase B: Ethanol:dichloromethane = 1:1; Flow rate: 20 mL/min; Gradient: 13% B to 13% B in 24 min; |
| | 1-(4-chloro-2-fluorophenyl)-N-(5-methyl-1- | | Wave Length: 220/254 nm. |
| | (1H-tetrazol-5-yl)azepan-3-yl)cyclopropane-1- | | Chiral separation conditions for assumed trans |
| | carboxamide | | isomers, 56B* and 56C *: CHIRALPAK IG, |
| | | | 2*25 cm, 5 um; Mobile Phase A: Hexanes, |
| | | | Mobile Phase B: Ethanol; Flow rate: 20 mL/min; |
| | | | Gradient: 15% B to 15% B in 12 min; Wave |
| | | | Length: 220/254 nm. |
| 56A* | 1-(4-chloro-2-fluorophenyl)-N-((3R,5R)-5- | 393.1 | Compound 56A*: Chiral RT(min): 22.40 |
| | methyl-1-(1H-tetrazol-5-yl)azepan-3- | | |
| | yl)cyclopropane-1-carboxamide | | |
| 56B* | 1-(4-chloro-2-fluorophenyl)-N-((3S,5R)-5- | 393.1 | Compound 56B*: Chiral RT(min): 9.35 |
| | methyl-1-(1H-tetrazol-5-yl)azepan-3- | | |
| | yl)cyclopropane-1-carboxamide | | |
| 56C* | 1-(4-chloro-2-fluorophenyl)-N-((3R,5S)-5- | 393.1 | Compound 56C*: Chiral RT(min): 11.50 |
| | methyl-1-(1H-tetrazol-5-yl)azepan-3- | | |
| | yl)cyclopropane-1-carboxamide | | |
| 56D* | 1-(4-chloro-2-fluorophenyl)-N-((3S,5S)-5- | 393.1 | Compound 56D*: Chiral RT(min): 19.19 |
| | methy1-1-(1H-tetrazol-5-yl)azepan-3- | | |
| | yl)cyclopropane-1-carboxamide | | |
| Rac-57 | 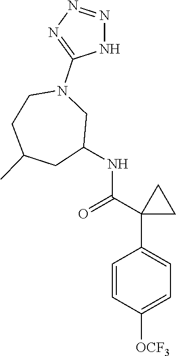 [SMILES: CC1CCN(c2nnn[nH]2)CC(NC(=O)C2(c3ccc(OC(F)(F)F)cc3)CC2)C1] | 425.0 | Rac-57, 57A*, 57B*, 57C*, and 57D* were prepared according to Protocol A and Example 1 using 5-methyl-1-(1H-tetrazol-5-yl)azepan-3- amine as the amine (i) reagent and 1-(4- trifluoromethoxyphenyl)cyclopropane-1- carboxylic acid as the carboxyl (ii) reagent. Rac-57 was then subjected to reverse phase column chromatography (C18 silica gel; mobile phase, acetonitrile in water, 0% to 100% gradient in 30 min; detector, UV 254 nm) to provide a mixture of compounds 57B* and 57C* as the first eluting peak, and a mixture of compounds 57A* and 57D* as the second eluting peak. Chiral separation conditions for assumed cis isomers, 57A* and 57D *: CHIRALPAK IG, 2*25 cm, 5 μm; Mobile Phase A: Hexanes (0.1% trifluoroacetic acid), Mobile Phase B: Ethanol:dichloromethane = 1:1; Flow rate: 20 mL/min; Gradient: 13% B to 13% B in 24 min; Wave Length: 220/254 nm. Chiral separation conditions for assumed trans |
| | N-(5-methyl-1-(1H-tetrazol-5-yl)azepan-3-yl)-1- | | isomers, 57B* and 57C *: CHIRALPAK IG, |
| | (4-(trifluoromethoxy)phenyl)cyclopropane-1- | | 2*25 cm, 5 μm; Mobile Phase A: Hexanes |
| | carboxamide | | (0.1% trifluoroacetic acid), Mobile Phase B: |
| | | | Ethanol:dichloromethane = 1:1; Flow rate: 20 |
| | | | mL/min; Gradient: 13% B to 13% B in 24 min; |
| | | | Wave Length: 220/254 nm. |
| 57A* | N-((3R,5R)-5-methyl-1-(1H-tetrazol-5- | 425.0 | Compound 57A*: Chiral RT(min): 13.63 |
| | yl)azepan-3-yl)-1-(4- | | |
| | (trifluoromethoxy)phenyl)cyclopropane-1- | | |
| | carboxamide | | |
| 57B* | N-((3S,5R)-5-methyl-1-(1H-tetrazol-5- | 425.0 | Compound 57B *: Chiral RT(min): 13.63 |
| | yl)azepan-3-yl)-1-(4- | | |
| | (trifluoromethoxy)phenyl)cyclopropane-1- | | |
| | carboxamide | | |
| 57C* | N-((3R,5S)-5-methyl-1-(1H-tetrazol-5- | 425.0 | Compound 57C*: Chiral RT(min): 11.02 |
| | yl)azepan-3-yl)-1-(4- | | |
| | (trifluoromethoxy)phenyl)cyclopropane-1- | | |
| | carboxamide | | |
| 57D* | N-((3S,5S)-5-methyl-1-(1H-tetrazol-5- | 425.0 | Compound 57D*: Chiral RT(min): 11.02 |
| | yl)azepan-3-yl)-1-(4- | | |
| | (trifluoromethoxy)phenyl)cyclopropane-1- | | |
| | carboxamide | | |
| Rac-58 | 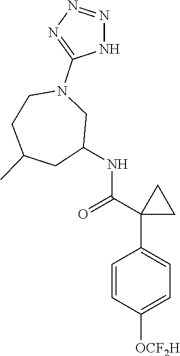 | 407.1 | Rac-58, 58A*, 58B*, 58C*, and 58D* were prepared according to Protocol A and Example 1 using 5-methyl-1-(1H-tetrazol-5-yl)azepan-3- amine as the amine (i) reagent and 1-(4- difluoromethoxyphenyl)cyclopropane-1- carboxylic acid as the carboxyl (ii) reagent. Rac-58 was then subjected to prep chiral HPLC using the following conditions: Xselect CSH C18 OBD Column 30*150 mm 5 μm; Mobile Phase A: acetonitrile, Mobile Phase B: Water (0.1% formic acid); Flow rate: 60 mL/min; Gradient: 33% B to 43% B in 10 min, 43% B; Wave Length: 254/220 nm to provide a mixture of compounds 58B* and 58C* as the first eluting peak, and a mixture of compounds 58A* and 58D* as the second eluting peak. Chiral separation conditions for assumed cis isomers, 58A* and 58D *: CHIRAL ART Cellulose-SC, 2*25 cm, 5 μm; Mobile Phase A: Hexanes, Mobile Phase B: Ethanol; Flow rate: 20 mL/min; Gradient: 15% B to 15% B in 21 min; Wave Length: 220/254 nm. |
| | 1-(4-(difluoromethoxy)phenyl)-N-(5-methyl-1- | | Chiral separation conditions for assumed trans |
| | (1H-tetrazol-5-yl)azepan-3-yl)cyclopropane-1- | | isomers, 58B* and 58C *: CHIRAL ART |
| | carboxamide | | Cellulose-SC, 2*25 cm, 5 μm; Mobile Phase A: |
| | | | Hex, Mobile Phase B: Ethanol; Flow rate: 20 |
| | | | mL/min; Gradient: 15% B to 15% B in 21 min; |
| | | | Wave Length: 220/254 nm. |
| 58A* | 1-(4-(difluoromethoxy)phenyl)-N-((3R,5R)-5- | 407.1 | Compound 58A*: Chiral RT(min): 18.43 |
| | methyl-1-(1H-tetrazol-5-yl)azepan-3- | | |
| | yl)cyclopropane-1-carboxamide | | |
| 58B* | 1-(4-(difluoromethoxy)phenyl)-N-((3S,5R)-5- | 407.1 | Compound 58B*: Chiral RT(min): 18.43 |
| | methyl-1-(1H-tetrazol-5-yl)azepan-3- | | |
| | yl)cyclopropane-1-carboxamide | | |
| 58C* | 1-(4-(difluoromethoxy)phenyl)-N-((3R,5S)-5- | 407.1 | Compound 58C*: Chiral RT(min): 15.22 |
| | methyl-1-(1H-tetrazol-5-yl)azepan-3- | | |
| | yl)cyclopropane-1-carboxamide | | |
| 58D* | 1-(4-(difluoromethoxy)phenyl)-N-((3S,5S)-5- | 407.1 | Compound 58D*: Chiral RT(min): 15.22 |
| | methyl-1-(1H-tetrazol-5-yl)azepan-3- | | |
| | yl)cyclopropane-1-carboxamide | | |
| Rac-59 | 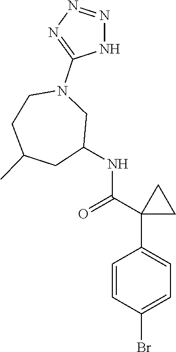 [SMILES: CC1CCN(c2nnn[nH]2)CC(NC(=O)C2(c3ccc(Br)cc3)CC2)C1] | 419.1 | Rac-59, 59A*, 59B*, 59C*, and 59D* were prepared according to Protocol A and Example 1 using 5-methyl-1-(1H-tetrazol-5-yl)azepan-3- amine as the amine (i) reagent and 1-(4- difluoromethoxyphenyl)cyclopropane-1- carboxylic acid as the carboxyl (ii) reagent. Rac-59 was then subjected to reverse phase column chromatography (C18 silica gel; mobile phase, acetonitrile in Water, 0% to 100% gradient in 30 min; detector, UV 254 nm) to provide a mixture of compounds 59B* and 59C* as the first eluting peak, and a mixture of compounds 59A* and 59D* as the second eluting peak. Chiral separation conditions for assumed cis isomers, 59A* and 59D *: Lux 5um Cellulose-4, 2.12*25 cm, 5 μm; Mobile Phase A: Hexanes (0.1% trifluoroacetic acid), Mobile Phase B: Ethanol; Flow rate: 20 mL/min; Gradient: 50% B to 50% B in 8 min; Wave Length: 220/254 nm. Chiral separation conditions for assumed trans |
| | 1-(4-bromophenyl)-N-(5-methyl-1-(1H-tetrazol- | | isomers, 59B* and 59C *: CHIRAL ART |
| | 5-yl)azepan-3-yl)cyclopropane-1-carboxamide | | Cellulose-SC, 2*25 cm, 5 μm; Mobile Phase A: |
| | | | Hexanes, Mobile Phase B: Ethanol; Flow rate: |
| | | | 20 mL/min; Gradient: 15% B to 15% B in 21 |
| | | | min; Wave Length: 220/254 nm. |
| 59A* | 1-(4-bromophenyl)-N-((3R,5R)-5-methyl-1- | 419.1 | Compound 59A*: Chiral RT(min): 5.46 |
| | (1H-tetrazol-5-yl)azepan-3-yl)cyclopropane-1- | | |
| | carboxamide | | |
| 59B* | 1-(4-bromophenyl)-N-((3S,5R)-5-methyl-1-(1H- | 419.1 | Compound 59B*: Chiral RT(min): 5.46 |
| | tetrazol-5-yl)azepan-3-yl)cyclopropane-1- | | |
| | carboxamide | | |
| 59C* | 1-(4-bromophenyl)-N-((3R,5S)-5-methyl-1-(1H- | 419.1 | Compound 59C*: Chiral RT(min): 6.61 |
| | tetrazol-5-yl)azepan-3-yl)cyclopropane-1- | | |
| | carboxamide | | |
| 59D* | 1-(4-bromophenyl)-N-((3S,5S)-5-methyl-1-(1H- | 419.1 | Compound 59D*: Chiral RT(min): 6.61 |
| | tetrazol-5-yl)azepan-3-yl)cyclopropane-1- | | |
| | carboxamide | | |
| Rac-60 | 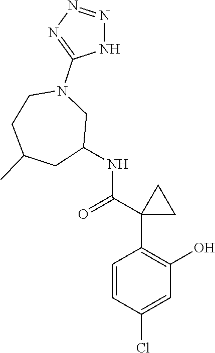 [SMILES: CC1CCN(c2nnn[nH]2)CC(NC(=O)C2(c3ccc(Cl)cc3O)CC2)C1] | 391.2 | Rac-60, 60A*, 60B*, 60C*, 60D*, and the corresponding protected —OMe phenol compounds, were prepared according to Protocol A and Example 1 using 5-methyl-1- (1H-tetrazol-5-yl)azepan-3-amine as the amine (i) reagent and 1-(4- difluoromethoxyphenyl)cyclopropane-1- carboxylic acid as the carboxyl (ii) reagent. Rac-60 was then subjected to reverse phase column chromatography (C18 silica gel; mobile phase, acetonitrile in Water, 0% to 100% gradient in 30 min; detector, UV 254 nm) to provide a mixture of compounds 60B* and 60C* as the first eluting peak, and a mixture of compounds 60A* and 60D* as the second eluting peak. Chiral separation conditions for assumed cis isomers, 60A* and 60D *: Xselect CSH C18 OBD Column 30*150 mm 5 μm; Mobile Phase A: acetonitrile, Mobile Phase B: Water (0.05% trifluoroacetic acid); Flow rate: 60 mL/min; Gradient: 29% B to 39% B in 8 min, 39% B to |
| | 1-(4-chloro-2-hydroxyphenyl)-N-(5-methyl-1- | | 44% B in 12 min, 44% B; Wave Length: |
| | (1H-tetrazol-5-yl)azepan-3-yl)cyclopropane-1- | | 254/220 nm. |
| | carboxamide | | Chiral separation conditions for assumed trans |
| | | | isomers, 60B* and 60C *: Lux 5um Cellulose-4, |
| | | | 2.12*25 cm, 5 μm; Mobile Phase A: Hexanes |
| | | | (0.1% trifluoroacetic acid), Mobile Phase B: |
| | | | ISOPROPANOL; Flow rate: 20 mL/min; |
| | | | Gradient: 25% B to 25% B in 18 min; Wave |
| | | | Length: 220/254 nm. |
| 60A* | 1-(4-chloro-2-hydroxyphenyl)-N-((3R,5R)-5- | 391.2 | Compound 60A *: Chiral RT(min): 7.50 |
| | methyl-1-(1H-tetrazol-5-yl)azepan-3- | | |
| | yl)cyclopropane-1-carboxamide | | |
| 60B* | 1-(4-chloro-2-hydroxyphenyl)-N-((3S,5R)-5- | 391.2 | Compound 60B *: Chiral RT(min): 11.53 |
| | methyl-1-(1H-tetrazol-5-yl)azepan-3- | | |
| | yl)cyclopropane-1-carboxamide | | |
| 60C* | 1-(4-chloro-2-hydroxyphenyl)-N-((3R,5S)-5- | 391.2 | Compound 60C *: Chiral RT(min): 15.58 |
| | methyl-1-(1H-tetrazol-5-yl)azepan-3- | | |
| | yl)cyclopropane-1-carboxamide | | |
| 60D* | 1-(4-chloro-2-hydroxyphenyl)-N-((3S,5S)-5- | 391.2 | Compound 60D *: Chiral RT(min): 11.57 |
| | methyl-1-(1H-tetrazol-5-yl)azepan-3- | | |
| | yl)cyclopropane-1-carboxamide | | |
| Rac-61 | 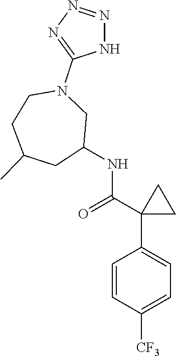 [SMILES: CC1CCN(c2nnn[nH]2)CC(NC(=O)C2(c3ccc(C(F)(F)F)cc3)CC2)C1] | 409.3 | Rac-61, 61A*, 61B*, 61C*, 61D*, and the corresponding protected —OMe phenol compounds, were prepared according to Protocol A and Example 1 using 5-methyl-1- (1H-tetrazol-5-yl)azepan-3-amine as the amine (i) reagent and 1-(4- trifluoromethylphenyl)cyclopropane-1- carboxylic acid as the carboxyl (ii) reagent. Rac-61 was then subjected to reverse phase column chromatography (Xselect CSH C18 OBD Column 30*150 mm 5 μm, Mobile Phase A: acetonitrile, Mobile Phase B: Water (0.05% trifluoroacetic acid); Flow rate: 60 mL/min; Gradient: 27% B to 37% B in 10 min; Wave Length: 254/220 nm) to provide a mixture of compounds 61B* and 61C* as the first eluting peak, and a mixture of compounds 61A* and 61D* as the second eluting peak. Chiral separation conditions for assumed cis isomers, 61A* and 61D *: Lux 5 um Cellulose-4, 2.12*25 cm, 5 μm; Mobile Phase A: Hexanes (0.1% trifluoroacetic acid), Mobile Phase B: |
| | N-(5-methyl-1-(1H-tetrazol-5-yl)azepan-3-yl)-1- | | Ethanol;Flow rate: 20 mL/min; Gradient: 20% |
| | (4-(trifluoromethyl)phenyl)cyclopropane-1- | | B to 20% B in 13 min; Wave Length: 220/254 |
| | carboxamide | | nm. |
| | | | Chiral separation conditions for assumed trans |
| | | | isomers, 61B* and 61C *: CHIRAL ART |
| | | | Cellulose-SC, 2*25 cm, 5 μm; Mobile Phase A: |
| | | | Hexanes (0.1% trifluoroacetic acid), Mobile |
| | | | Phase B: Ethanol; Flow rate: 20 mL/min; |
| | | | Gradient: 15% B to 15% B in 20 min; Wave |
| | | | Length: 220/254 nm. |
| 61A* | N-((3R,5R)-5-methyl-1-(1H-tetrazol-5- | 409.3 | Compound 61A *: Chiral RT(min): 12.49 |
| | yl)azepan-3-yl)-1-(4- | | |
| | (trifluoromethyl)phenyl)cyclopropane-1- | | |
| | carboxamide | | |
| 61B* | N-((3R,5S)-5-methyl-1-(1H-tetrazol-5- | 409.3 | Compound 61B *: Chiral RT(min): 14.44 |
| | yl)azepan-3-yl)-1-(4- | | |
| | (trifluoromethyl)phenyl)cyclopropane-1- | | |
| | carboxamide | | |
| 61C* | N-((3S,5R)-5-methyl-1-(1H-tetrazol-5- | 409.3 | Compound 61C *: Chiral RT(min): 17.44 |
| | yl)azepan-3-yl)-1-(4- | | |
| | (trifluoromethyl)phenyl)cyclopropane-1- | | |
| | carboxamide | | |
| 61D* | N-((3S,5S)-5-methy1-1-(1H-tetrazol-5- | 409.3 | Compound 61D *: Chiral RT(min): 9.44 |
| | yl)azepan-3-yl)-1-(4- | | |
| | (trifluoromethyl)phenyl)cyclopropane-1- | | |
| | carboxamide | | |
| Rac-62 | 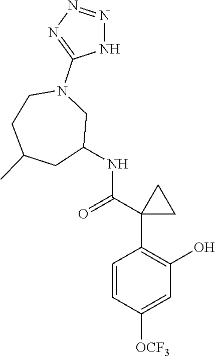 [SMILES: CC1CCN(c2nnn[nH]2)CC(NC(=O)C2(c3ccc(OC(F)(F)F)cc3O)CC2)C1] | 421.2 | Rac-62, 62A*, 62B*, 62C*, 62D*, and the corresponding protected —OMe phenol compounds, were prepared according to Protocol A and Example 1 using 5-methyl-1- (1H-tetrazol-5-yl)azepan-3-amine as the amine (i) reagent and 1-(4- difluoromethoxyphenyl)cyclopropane-1- carboxylic acid as the carboxyl (ii) reagent. Rac-62 was then subjected to reverse phase column chromatography (C18 silica gel; mobile phase, Acetonitrile in water, 0% to 100% gradient in 30 min; detector, UV 254 nm) to provide a mixture of compounds 62A* and 62D* as the first eluting peak, and a mixture of compounds 62B* and 62C* as the second eluting peak. Chiral separation conditions for assumed cis isomers, 62A* and 62D *: CHIRAL ART Cellulose-SC, 2*25 cm, 5 μm; Mobile Phase A: Hexanes (0.1% trifluoroacetic acid), Mobile Phase B: Ethanol:dichloromethane = 1:1; Flow rate: 20 mL/min; Gradient: 10% B to 10% B in |
| | 1-(2-hydroxy-4-(trifluoromethoxy)phenyl)-N- | | 28 min; Wave Length: 220/254 nm. |
| | (5-methyl-1-(1H-tetrazol-5-yl)azepan-3- | | Chiral separation conditions for assumed trans |
| | yl)cyclopropane-1-carboxamide | | isomers, 62B* and 62C *: Lux 5 um Cellulose-4, |
| | | | 2.12*25 cm, 5 μm; Mobile Phase A: Hexanes |
| | | | (0.1% trifluoroacetic acid), Mobile Phase B: |
| | | | isopropanol; Flow rate: 20 mL/min; Gradient: |
| | | | 25% B to 25% B in 18 min; Wave Length: |
| | | | 220/254 nm. |
| 62A* | 1-(2-hydroxy-4-(trifluoromethoxy)phenyl)-N- | 421.2 | Compound 62A *: Chiral RT(min): 37.24 |
| | ((3R,5R)-5-methyl-1-(1H-tetrazol-5-yl)azepan- | | |
| | 3-yl)cyclopropane-1-carboxamide | | |
| 62B* | 1-(2-hydroxy-4-(trifluoromethoxy)phenyl)-N- | 421.2 | Compound 62B *: Chiral RT(min): 20.92 |
| | ((3S,5R)-5-methyl-1-(1H-tetrazol-5-yl)azepan- | | |
| | 3-yl)cyclopropane-1-carboxamide | | |
| 62C* | 1-(2-hydroxy-4-(trifluoromethoxy)phenyl)-N- | 421.2 | Compound 62C *: Chiral RT(min): 24.11 |
| | ((3R,5S)-5-methyl-1-(1H-tetrazol-5-yl)azepan- | | |
| | 3-yl)cyclopropane-1-carboxamide | | |
| 62D* | 1-(2-hydroxy-4-(trifluoromethoxy)phenyl)-N- | 421.2 | Compound 62D *: Chiral RT(min): 32.52 |
| | ((3S,5R)-5-methy1-1-(1H-tetrazol-5-yl)azepan- | | |
| | 3-yl)cyclopropane-1-carboxamide | | |
| Rac-63 | 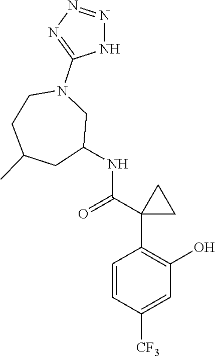 [SMILES: CC1CCN(c2nnn[nH]2)CC(NC(=O)C2(c3ccc(C(F)(F)F)cc3O)CC2)C1] | 425.2 | Rac-63, 63A*, 63B*, 63C*, 63D*, and the corresponding protected —OMe phenol compounds, were prepared according to Protocol A and Example 1 using 5-methyl-1- (1H-tetrazol-5-yl)azepan-3-amine as the amine (i) reagent and 1-(4- difluoromethoxyphenyl)cyclopropane-1- carboxylic acid as the carboxyl (ii) reagent. Rac-63 was then subjected to reverse phase column chromatography (C18 silica gel; mobile phase, Acetonitrile in Water, 0% to 100% gradient in 30 min; detector, UV 254 nm) to provide a mixture of compounds 63B* and 63C* as the first eluting peak, and a mixture of compounds 63A* and 63D* as the second eluting peak. Chiral separation conditions for assumed cis isomers, 63A* and 63D *: CHIRAL ART Cellulose-SC, 2*25 cm, 5 μm; Mobile Phase A: Hexanes (0.1% trifluoroacetic acid), Mobile Phase B: isopropanol; Flow rate: 20 mL/min; Gradient: 20% B to 20% B in 24 min; Wave |
| | 1-(2-hydroxy-4-(trifluoromethyl)phenyl)-N-(5- | | Length: 220/254 nm. |
| | methyl-1-(1H-tetrazol-5-yl)azepan-3- | | Chiral separation conditions for assumed trans |
| | yl)cyclopropane-1-carboxamide | | isomers, 63B* and 63C *: Lux 5 um Cellulose-4, |
| | | | 2.12*25 cm, 5 μm; Mobile Phase A: Hexanes |
| | | | (0.1% trifluoroacetic acid), Mobile Phase B: |
| | | | isopropanol; Flow rate: 20 mL/min; Gradient: |
| | | | 30% B to 30% B in 10 min; Wave Length: |
| | | | 220/254 nm. |
| 63A* | 1-(2-hydroxy-4-(trifluoromethyl)phenyl)-N- | 425.2 | Compound 63A*: Chiral RT(min): 20.84 |
| | ((3R,5R)-5-methyl-1-(1H-tetrazol-5-yl)azepan- | | |
| | 3-yl)cyclopropane-1-carboxamide | | |
| 63B* | 1-(2-hydroxy-4-(trifluoromethyl)phenyl)-N- | 425.2 | Compound 63B*: Chiral RT(min): 6.53 |
| | ((3S,5R)-5-methyl-1-(1H-tetrazol-5-yl)azepan- | | |
| | 3-yl)cyclopropane-1-carboxamide | | |
| 63C* | 1-(2-hydroxy-4-(trifluoromethyl)phenyl)-N- | 425.2 | Compound 63C*: Chiral RT(min): 8.83 |
| | ((3R,5S)-5-methyl-1-(1H-tetrazol-5-yl)azepan- | | |
| | 3-yl)cyclopropane-1-carboxamide | | |
| 63D* | 1-(2-hydroxy-4-(trifluoromethyl)phenyl)-N- | 425.2 | Compound 63D*: Chiral RT(min): 15.31 |
| | ((3S,5S)-5-methyl-1-(1H-tetrazol-5-yl)azepan- | | |
| | 3-yl)cyclopropane-1-carboxamide | | |
| Rac-64 | 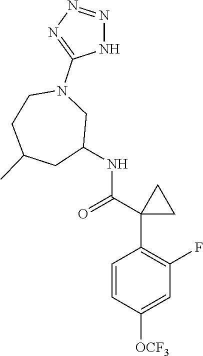 [SMILES: CC1CCN(c2nnn[nH]2)CC(NC(=O)C2(c3ccc(OC(F)(F)F)cc3F)CC2)C1] | 443.3 | Rac-64, 64A*, 64B*, 64C*, 64D*, and the corresponding protected —OMe phenol compounds, were prepared according to Protocol A and Example 1 using 5-methyl-1- (1H-tetrazol-5-yl)azepan-3-amine as the amine (i) reagent and 1-(2-fluoro-4- (trifluoromethoxy)phenyl)cyclopropane-1- carboxylic acid as the carboxyl (ii) reagent. Rac-64 was then subjected to reverse phase column chromatography (CHIRAL ART Cellulose-SB, 2*25 cm, 5 μm; Mobile Phase A: Hexanes (0.1% trifluoroacetic acid), Mobile Phase B: isopropanol:dichloromethane = 1:1; Flow rate: 20 mL/min; Gradient: 15% B to 15% B in 13 min; Wave Length: 220/254 nm) to provide a mixture of compounds 64B* and 64C* as the first eluting peak, and a mixture of compounds 64A* and 64D* as the second eluting peak. Chiral separation conditions for assumed cis isomers, 64A* and 64D *: Lux 5 um Cellulose-4, 2.12*25 cm, 5 μm Mobile Phase A: Hexanes |
| | 1-(2-fluoro-4-(trifluoromethoxy)phenyl)-N-(5- | | (0.1% trifluoroacetic acid), Mobile Phase B: |
| | methyl-1-(1H-tetrazol-5-yl)azepan-3- | | isopropanol; Flow rate: 20 mL/min; Gradient: |
| | yl)cyclopropane-1-carboxamide | | 30% B to 30% B in 12 min; Wave Length: |
| | | | 220/254 nm. |
| | | | Chiral separation conditions for assumed trans |
| | | | isomers, 64B* and 64C *: CHIRAL ART |
| | | | Cellulose-SB, 2*25 cm, 5 μm; Mobile Phase A: |
| | | | Hexanes (0.1% trifluoroacetic acid), Mobile |
| | | | Phase B: isopropanol:dichloromethane = 1: 1; |
| | | | Flow rate: 20 mL/min; Gradient: 15% B to 15% |
| | | | B in 13 min; Wave Length: 220/254 nm. |
| 64A* | 1-(2-fluoro-4-(trifluoromethoxy)phenyl)-N- | 443.3 | Compound 64A*: Chiral RT(min): 9.85 |
| | ((3R,5R)-5-methyl-1-(1H-tetrazol-5-yl)azepan- | | |
| | 3-yl)cyclopropane-1-carboxamide | | |
| 64B* | 1-(2-fluoro-4-(trifluoromethoxy)phenyl)-N- | 443.3 | Compound 64B*: Chiral RT(min): 10.29 |
| | ((3S,5R)-5-methyl-1-(1H-tetrazol-5-yl)azepan- | | |
| | 3-yl)cyclopropane-1-carboxamide | | |
| 64C* | 1-(2-fluoro-4-(trifluoromethoxy)phenyl)-N- | 443.3 | Compound 64C*: Chiral RT(min): 12.22 |
| | ((3R,5S)-5-methyl-1-(1H-tetrazol-5-yl)azepan- | | |
| | 3-yl)cyclopropane-1-carboxamide | | |
| 64D* | 1-(2-fluoro-4-(trifluoromethoxy)phenyl)-N- | 443.3 | Compound 64D*: Chiral RT(min): 7.68 |
| | ((3S,5S)-5-methy1-1-(1H-tetrazol-5-yl)azepan- | | |
| | 3-yl)cyclopropane-1-carboxamide | | |
| Rac-65 | 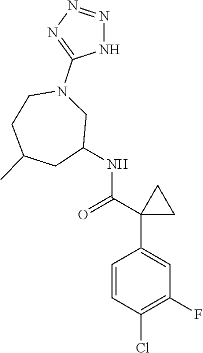 [SMILES: CC1CCN(c2nnn[nH]2)CC(NC(=O)C2(c3ccc(Cl)c(F)c3)CC2)C1] | 393.3 | Rac-65, 65A*, 65B*, 65C*, 65D*, and the corresponding protected —OMe phenol compounds, were prepared according to Protocol A and Example 1 using 5-methyl-1- (1H-tetrazol-5-yl)azepan-3-amine as the amine (i) reagent and 1-(3-fluoro-4- chlorophenyl)cyclopropane-1-carboxylic acid as the carboxyl (ii) reagent. Rac-65 was then subjected to reverse phase column chromatography (XBridge Prep OBD C18 Column, 30*150 mm, 5 μm; Mobile Phase A: Water (10 mmol/L NH₄HCO₃), Mobile Phase B: acetonitrile; Flow rate: 60 mL/min; Gradient: 27% B to 37% B in 10 min, 37% B; Wave Length: 220 nm) to provide a mixture of compounds 65B* and 65C* as the first eluting peak, and a mixture of compounds 65A* and 65D* as the second eluting peak. Chiral separation conditions for assumed cis isomers, 65A* and 65D *: Lux 5 um Cellulose-4, 2.12*25 cm, 5 μm Mobile Phase A: Hexanes (0.1% trifluoroacetic acid), Mobile Phase B: |
| | 1-(4-chloro-3-fluorophenyl)-N-(5-methyl-1- | | isopropanol; Flow rate: 20 mL/min; Gradient: |
| | (1H-tetrazol-5-yl)azepan-3-yl)cyclopropane-1- | | 50% B to 50% B in 10.5 min; Wave Length: |
| | carboxamide | | 220/254 nm. |
| | | | Chiral separation conditions for assumed trans |
| | | | isomers, 65B* and 65C *: CHIRALPAK IG, |
| | | | 2*25 cm, 5 μm Mobile Phase A: IPA(0.1% |
| | | | trifluoroacetic acid), Mobile Phase B: |
| | | | isopropanol; Flow rate: 20 mL/min; Gradient: |
| | | | 15% B to 15% B in 27 min; Wave Length: |
| | | | 220/254 nm. |
| 65A* | 1-(4-chloro-3-fluorophenyl)-N-((3R,5R)-5- | 393.3 | Compound 65A *: Chiral RT(min): 9.37 |
| | methy1-1-(1H-tetrazol-5-yl)azepan-3- | | |
| | yl)cyclopropane-1-carboxamide | | |
| 65B* | 1-(4-chloro-3-fluorophenyl)-N-((3S,5R)-5- | 393.3 | Compound 65B *: Chiral RT(min): 17.12 |
| | methyl-1-(1H-tetrazol-5-yl)azepan-3- | | |
| | yl)cyclopropane-1-carboxamide | | |
| 65C* | 1-(4-chloro-3-fluorophenyl)-N-((3R,5S)-5- | 393.3 | Compound 65C *: Chiral RT(min): 22.67 |
| | methyl-1-(1H-tetrazol-5-yl)azepan-3- | | |
| | yl)cyclopropane-1-carboxamide | | |
| 65D* | 1-(4-chloro-3-fluorophenyl)-N-((3S,5S)-5- | 393.3 | Compound 65D *: Chiral RT(min): 7.17 |
| | methyl-1-(1H-tetrazol-5-yl)azepan-3- | | |
| | yl)cyclopropane-1-carboxamide | | |
| Rac-66 | 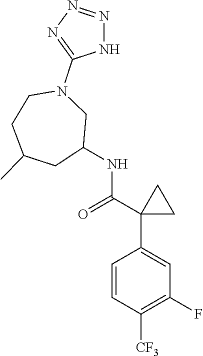 [SMILES: CC1CCN(c2nnn[nH]2)CC(NC(=O)C2(c3ccc(C(F)(F)F)c(F)c3)CC2)C1] | 427.2 | Rac-66, 66A*, 66B*, 66C*, and 66D* were prepared according to Protocol A and Example 1 using 5-methyl-1-(1H-tetrazol-5-yl)azepan-3- amine as the amine (i) reagent and 1-(3-fluoro- 4-trifluoromethylphenyl)cyclopropane-1- carboxylic acid as the carboxyl (ii) reagent. Rac-66 was then subjected to reverse phase column chromatography (YMC-Actus Triart C18 ExRS, 30*150 mm, 5 μm; Mobile Phase A: Water (10 mmol/L NH₄HCO₃), Mobile Phase B: acetonitrile; Flow rate: 60 mL/min; Gradient: 24% B to 34% B in 10 min, 34% B; Wave Length: 254 nm) to provide a mixture of compounds 66A* and 66B* as the first eluting peak, and a mixture of compounds 66B* and 66C* as the second eluting peak. Chiral separation conditions for assumed cis isomers, 66A* and 66D *: Lux 5 um Cellulose-4, 2.12*25 cm, 5 μm; Mobile Phase A: Hexanes (0.1% trifluoroacetic acid), Mobile Phase B: Methanol:ethanol = 1:1; Flow rate: 20 mL/min; Gradient: 15% B to 15% B in 11.5 |
| | 1-(3-fluoro-4-(trifluoromethyl)phenyl)-N-(5- | | min; Wave Length: 220/254 nm. |
| | methyl-1-(1H-tetrazol-5-yl)azepan-3- | | Chiral separation conditions for assumed trans |
| | yl)cyclopropane-1-carboxamide | | isomers, 66B* and 66C *: Lux 5 um Cellulose-4, |
| | | | 2.12*25 cm, 5 μm; Mobile Phase A: Hexanes |
| | | | (0.1% trifluoroacetic acid), Mobile Phase B: |
| | | | isopropanol; Flow rate: 20 mL/min; Gradient: |
| | | | 30% B to 30% B in 15 min; Wave Length: |
| | | | 220/254 nm. |
| 66A* | 1-(3-fluoro-4-(trifluoromethyl)phenyl)-N- | 427.2 | Compound 66A*: Chiral RT(min): 10.86 |
| | ((3R,5R)-5-methyl-1-(1H-tetrazol-5-yl)azepan- | | |
| | 3-yl)cyclopropane-1-carboxamide | | |
| 66B* | 1-(3-fluoro-4-(trifluoromethyl)phenyl)-N- | 427.2 | Compound 66B*: Chiral RT(min): 8.78 |
| | ((3S,5R)-5-methy1-1-(1H-tetrazol-5-yl)azepan- | | |
| | 3-yl)cyclopropane-1-carboxamide | | |
| 66C* | 1-(3-fluoro-4-(trifluoromethyl)phenyl)-N- | 427.2 | Compound 66C*: Chiral RT(min): 12.49 |
| | ((3R,5S)-5-methyl-1-(1H-tetrazol-5-yl)azepan- | | |
| | 3-yl)cyclopropane-1-carboxamide | | |
| 66D* | 1-(3-fluoro-4-(trifluoromethyl)phenyl)-N- | 427.2 | Compound 66D*: Chiral RT(min): 9.60 |
| | ((3S,5S)-5-methy1-1-(1H-tetrazol-5-yl)azepan- | | |
| | 3-yl)cyclopropane-1-carboxamide | | |
| Rac-67 | 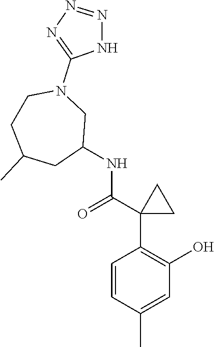 [SMILES: Cc1ccc(C2(C(=O)NC3CC(C)CCN(c4nnn[nH]4)C3)CC2)c(O)c1] | 371.1 | Rac-67, 67A*, 67B*, 67C*, 67D*, and the corresponding protected —OMe phenol compounds, were prepared according to Protocol A and Example 1 using 5-methyl-1- (1H-tetrazol-5-yl)azepan-3-amine as the amine (i) reagent and 1-(2-methoxy-4- methylphenyl)cyclopropane-1-carboxylic acid as the carboxyl (ii) reagent. Rac-67 was then subjected to reverse phase column chromatography (XSelect CSH Prep C18 OBD Column, 19*250 mm, 5 μm; Mobile Phase A: Water(0.05% trifluoroacetic acid), Mobile Phase B: MeOH; Flow rate: 20 mL/min; Gradient: 59% B to 59% B in 8 min, 59% B; Wave Length: 254 nm) to provide a mixture of compounds 67A* and 67D* as the first eluting peak, and a mixture of compounds 67B* and 67C* as the second eluting peak. Chiral separation conditions for assumed cis isomers, 67A* and 67D *: Lux 5 um Cellulose-4, 2.12*25 cm, 5 μm Mobile Phase A: Hexanes |
| | 1-(2-hydroxy-4-methylphenyl)-N-(5-methyl-1- | | (0.1% trifluoroacetic acid), Mobile Phase B: |
| | (1H-tetrazol-5-yl)azepan-3-yl)cyclopropane-1- | | isopropanol; Flow rate: 20 mL/min; Gradient: |
| | carboxamide | | 50% B to 50% B in 10 min; Wave Length: |
| | | | 220/254 nm. |
| | | | Chiral separation conditions for assumed trans |
| | | | isomers, 67B* and 67C *: CHIRALPAK IG, |
| | | | 2*25 cm, 5 μm; Mobile Phase A: Hexanes |
| | | | (0.1% trifluoroacetic acid), Mobile Phase B: |
| | | | isopropanol; Flow rate: 20 mL/min; Gradient: |
| | | | 10% B to 10% B in 23 min; Wave Length: |
| | | | 220/254 nm. |
| 67A* | 1-(2-hydroxy-4-methylphenyl)-N-((3R,5R)-5- | 371.1 | Compound 67A*: Chiral RT(min): 9.13 |
| | methyl-1-(1H-tetrazol-5-yl)azepan-3- | | |
| | yl)cyclopropane-1-carboxamide | | |
| 67B* | 1-(2-hydroxy-4-methylphenyl)-N-((3S,5R)-5- | 371.1 | Compound 67B*: Chiral RT(min): 16.14 |
| | methyl-1-(1H-tetrazol-5-yl)azepan-3- | | |
| | yl)cyclopropane-1-carboxamide | | |
| 67C* | 1-(2-hydroxy-4-methylphenyl)-N-((3R,5S)-5- | 371.1 | Compound 67C*: Chiral RT(min): 20.63 |
| | methyl-1-(1H-tetrazol-5-yl)azepan-3- | | |
| | yl)cyclopropane-1-carboxamide | | |
| 67D* | 1-(2-hydroxy-4-methylphenyl)-N-((3S,5S)-5- | 371.1 | Compound 67D*: Chiral RT(min): 7.37 |
| | methyl-1-(1H-tetrazol-5-yl)azepan-3- | | |
| | yl)cyclopropane-1-carboxamide | | |
| Rac-68 | 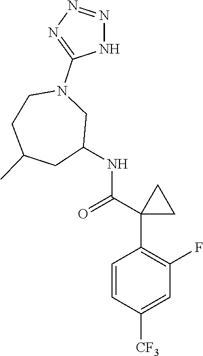 [SMILES: CC1CCN(c2nnn[nH]2)CC(NC(=O)C2(c3ccc(C(F)(F)F)cc3F)CC2)C1] | 427.2 | Rac-68, 68A*, 68B*, 68C*, and 68D* were prepared according to Protocol A and Example 1 using (3R,5R)-5-methyl-1-(1H-tetrazol-5- yl)azepan-3-amine as the amine (i) reagent and 1-(2-fluoro-4-trifluoromethylphenyl) cyclopropane-1-carboxylic acid as the carboxyl (ii) reagent. Rac-68 was then subjected to reverse phase column chromatography (YMC-Actus Triart C18 ExRS, 30*150 mm, 5 μm; Mobile Phase A: Water (10 mmol/L NH₄HCO₃), Mobile Phase B: acetonitrile; Flow rate: 60 mL/min; Gradient: 24% B to 34% B in 10 min, 34% B; Wave Length: 254/220 nm) to provide a mixture of compounds 68B* and 68C* as the first eluting peak, and a mixture of compounds 68A* and 68D* as the second eluting peak. Chiral separation conditions for assumed cis isomers, 68A* and 68D *: Lux 5 um Cellulose-4, 2.12*25 cm, 5um; Mobile Phase A: Hexanes (0.1% trifluoroacetic acid), Mobile Phase B: Methanol:ethanol = 1:1; Flow rate: 20 |
| | 1-(2-fluoro-4-(trifluoromethyl)phenyl)-N-(5- | | mL/min; Gradient: 15% B to 15% B in 11.5 |
| | methyl-1-(1H-tetrazol-5-yl)azepan-3- | | min; Wave Length: 220/254 nm. |
| | yl)cyclopropane-1-carboxamide | | Chiral separation conditions for assumed trans |
| | | | isomers, 68B* and 68C*: CHIRAL ART |
| | | | Cellulose-SB, 2*25 cm, 5 μm; Mobile Phase A: |
| | | | Hexanes (0.1% trifluoroacetic acid), Mobile |
| | | | Phase B: isopropanol:dichloromethane = 1:1; |
| | | | Flow rate: 20 mL/min; Gradient: 15% B to 15% |
| | | | B in 14 min; Wave Length: 220/254 nm. |
| 68A* | 1-(2-fluoro-4-(trifluoromethyl)phenyl)-N- | 427.2 | Compound 68A *: Chiral RT(min): 9.60 |
| | ((3R,5R)-5-methyl-1-(1H-tetrazol-5-yl)azepan- | | |
| | 3-yl)cyclopropane-1-carboxamide | | |
| 68B* | 1-(2-fluoro-4-(trifluoromethyl)phenyl)-N- | 427.2 | Compound 68B *: Chiral RT(min): 11.20 |
| | ((3S,5R)-5-methy1-1-(1H-tetrazol-5-yl)azepan- | | |
| | 3-yl)cyclopropane-1-carboxamide | | |
| 68C* | 1-(2-fluoro-4-(trifluoromethyl)phenyl)-N- | 427.2 | Compound 68C *: Chiral RT(min): 13.43 |
| | ((3R,5S)-5-methyl-1-(1H-tetrazol-5-yl)azepan- | | |
| | 3-yl)cyclopropane-1-carboxamide | | |
| 68D* | 1-(2-fluoro-4-(trifluoromethyl)phenyl)-N- | 427.2 | Compound 68D *: Chiral RT(min): 10.86 |
| | ((3S,5S)-5-methyl-1-(1H-tetrazol-5-yl)azepan- | | |
| | 3-yl)cyclopropane-1-carboxamide | | |
| Rac-69 | 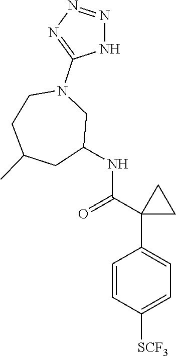 | 441.2 | Rac-69, 69A*, 69B*, 69C*, and 69D* were prepared according to Protocol A and Example 1 using 5-methyl-1-(1H-tetrazol-5-yl)azepan-3- amine as the amine (i) reagent and 1-(2-fluoro- 4-trifluoromethylphenyl)cyclopropane-1- carboxylic acid as the carboxyl (ii) reagent. Rac-69 was then subjected to reverse phase column chromatography (C18 silica gel; mobile phase, Acetonitrile in Water, 0% to 100% gradient in 30 min; detector, UV 254 nm) to provide a mixture of compounds 69B* and 69C* as the first eluting peak, and a mixture of compounds 69A* and 69D* as the second eluting peak. Chiral separation conditions for assumed cis isomers, 69A* and 69D*: Lux 5 um Cellulose-4, 2.12*25 cm, 5 μm; Mobile Phase A: Hexanes (0.1% trifluoroacetic acid), Mobile Phase B: isopropanol; Flow rate: 20 mL/min; Gradient: 45% B to 45% B in 10 min; Wave Length: 254/220 nm. Chiral separation conditions for assumed trans |
| | N-(5-methyl-1-(1H-tetrazol-5-yl)azepan-3-yl)-1- | | isomers, 69B* and 69C*: CHIRAL ART |
| | (4-((trifluoromethyl)thio)phenyl)cyclopropane- | | Cellulose-SC, 2*25 cm, 5 μm; Mobile Phase A: |
| | 1-carboxamide | | Hexanes (0.1% trifluoroacetic acid), Mobile |
| | | | Phase B: Ethanol; Flow rate: 20 mL/min; |
| | | | Gradient: 20% B to 20% B in 14 min; Wave |
| | | | Length: 220/254 nm. |
| 69A* | N-((3R,5R)-5-methyl-1-(1H-tetrazol-5- | 441.2 | Compound 69A*: Chiral RT(min): 8.23 |
| | yl)azepan-3-yl)-1-(4- | | |
| | ((trifluoromethyl)thio)phenyl)cyclopropane-1- | | |
| | carboxamide | | |
| 69B* | N-((3S,5R)-5-methyl-1-(1H-tetrazol-5- | 441.2 | Compound 69B*: Chiral RT(min): 11.45 |
| | yl)azepan-3-yl)-1-(4- | | |
| | ((trifluoromethyl)thio)phenyl)cyclopropane-1- | | |
| | carboxamide | | |
| 69C* | N-((3R,5S)-5-methyl-1-(1H-tetrazol-5- | 441.2 | Compound 69C*: Chiral RT(min): 14.22 |
| | yl)azepan-3-yl)-1-(4- | | |
| | ((trifluoromethyl)thio)phenyl)cyclopropane-1- | | |
| | carboxamide | | |
| 69D* | N-((3S,5S)-5-methyl-1-(1H-tetrazol-5- | 441.2 | Compound 69D*: Chiral RT(min): 5.69 |
| | yl)azepan-3-yl)-1-(4- | | |
| | ((trifluoromethyl)thio)phenyl)cyclopropane-1- | | |
| | carboxamide | | |
| Rac-70 | 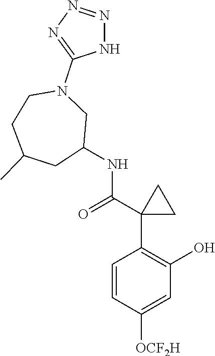 | 423.1 | Rac-70, 70A*, 70B*, 70C*, and 70D* were prepared according to Protocol A and Example 1 using 5-methyl-1-(1H-tetrazol-5-yl)azepan-3- amine as the amine (i) reagent and 1-(2-fluoro- 4-difluoromethylphenyl)cyclopropane-1- carboxylic acid as the carboxyl (ii) reagent. Rac-70 was then subjected to reverse phase column chromatography (XBridge Prep Phenyl OBD Column, 19*150 mm, 5 μm; Mobile Phase A: Water (10 mmol/L NH₄HCO₃), Mobile Phase B: acetonitrile; Flow rate: 60 mL/min; Gradient: 14% B to 24% B in 8 min, 24% B to 35% B in 15 min, 35% B; Wave Length: 220 nm) to provide a mixture of compounds 70B* and 70C* as the first eluting peak, and a mixture of compounds 70A* and 70D* as the second eluting peak. Chiral separation conditions for assumed cis isomers, 70A* and 70D *: Lux 5 um Cellulose-4, 2.12*25 cm, 5 μm; Mobile Phase A: Hexanes (0.1% trifluoroacetic acid), Mobile Phase B: isopropanol; Flow rate: 20 mL/min; Gradient: |
| | 1-(4-(difluoromethoxy)-2-hydroxyphenyl)-N-(5- | | 45% B to 45% B in 10 min; Wave Length: |
| | methyl-1-(1H-tetrazol-5-yl)azepan-3- | | 254/220 nm. |
| | yl)cyclopropane-1-carboxamide | | Chiral separation conditions for assumed trans |
| | | | isomers, 70B* and 70C *: CHIRAL ART |
| | | | Cellulose-SC, 2*25 cm, 5 um; Mobile Phase A: |
| | | | Hexanes (0.1% trifluoroacetic acid), Mobile |
| | | | Phase B: isopropanol:dichloromethane =1: 1; |
| | | | Flow rate: 20 mL/min; Gradient: 20% B to 20% |
| | | | B in 24 min; Wave Length: 220/254 nm. |
| 70A* | 1-(4-(difluoromethoxy)-2-hydroxyphenyl)-N- | 423.1 | Compound 70A *: Chiral RT(min): 12.18 |
| | ((3R,5R)-5-methyl-1-(1H-tetrazol-5-yl)azepan- | | |
| | 3-yl)cyclopropane-1-carboxamide | | |
| 70B* | 1-(4-(difluoromethoxy)-2-hydroxyphenyl)-N- | 423.1 | Compound 70B *: Chiral RT(min): 22.017 |
| | ((3S,5R)-5-methy1-1-(1H-tetrazol-5-yl)azepan- | | |
| | 3-yl)cyclopropane-1-carboxamide | | |
| 70C* | 1-(4-(difluoromethoxy)-2-hydroxyphenyl)-N- | 423.1 | Compound 70C *: Chiral RT(min): 16.263 |
| | ((3R,5S)-5-methy1-1-(1H-tetrazol-5-yl)azepan- | | |
| | 3-yl)cyclopropane-1-carboxamide | | |
| 70D* | 1-(4-(difluoromethoxy)-2-hydroxyphenyl)-N- | 423.1 | Compound 70D *: Chiral RT(min): 9.27 |
| | ((3S,5S)-5-methyl-1-(1H-tetrazol-5-yl)azepan- | | |
| | 3-yl)cyclopropane-1-carboxamide | | |
| Rac-71 | 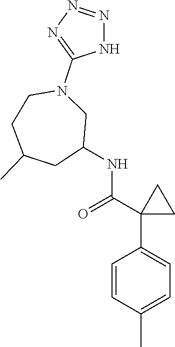 [SMILES: Cc1ccc(C2(C(=O)NC3CC(C)CCN(c4nnn[nH]4)C3)CC2)cc1] | 355.2 | Rac-71, 71A*, 71B*, 71C*, and 71D* were prepared according to Protocol A and Example 1 using 5-methyl-1-(1H-tetrazol-5-yl)azepan-3- amine as the amine (i) reagent and 1-(4- methylphenyl)cyclopropane-1-carboxylic acid as the carboxyl (ii) reagent. Rac-71 was then subjected to reverse phase column chromatography (XBridge Shield RP18 OBD Column 30*150 mm, 5 μm; Mobile Phase A: Water (10 mmol/L NH₄HCO₃), Mobile Phase B: acetonitrile; Flow rate: 60 mL/min; Gradient: 20% B to 30% B in 10 min; Wave Length: 254 nm) to provide a mixture of compounds 71B* and 71C* as the first eluting peak, and a mixture of compounds 71A* and 71D* as the second eluting peak. Chiral separation conditions for assumed cis isomers, 71A* and 71D *: Lux 5 um Cellulose-4, 2.12*25 cm, 5 μm Mobile Phase A: Hexanes (0.1% trifluoroacetic acid), Mobile Phase B: Methanol:ethanol = 1:1; Flow rate: 20 |
| | N-(5-methyl-1-(1H-tetrazol-5-yl)azepan-3-yl)-1- | | mL/min; Gradient: 50% B to 50% B in 8 min; |
| | (p-tolyl)cyclopropane-1-carboxamide | | Wave Length: 220/254 nm. |
| | | | Chiral separation conditions for assumed trans |
| | | | isomers, 71B* and 71C *: Lux 5 um Cellulose-4, |
| | | | 2.12*25 cm, 5 μm Mobile Phase A: Hexanes |
| | | | (0.1% trifluoroacetic acid), Mobile Phase B: |
| | | | isopropanol; Flow rate: 20 mL/min; Gradient: |
| | | | 50% B to 50% B in 13 min; Wave Length: |
| | | | 220/254 nm. |
| 71A* | N-((3R,5R)-5-methyl-1-(1H-tetrazol-5- | 355.2 | Compound 71A*: Chiral RT(min): 6.347 |
| | yl)azepan-3-yl)-1-(p-tolyl)cyclopropane-1- | | |
| | carboxamide | | |
| 71B* | N-((3S,5R)-5-methyl-1-(1H-tetrazol-5- | 355.2 | Compound 71B*: Chiral RT(min): 20.64 |
| | yl)azepan-3-yl)-1-(p-tolyl)cyclopropane-1- | | |
| | carboxamide | | |
| 71C* | N-((3R,5S)-5-methyl-1-(1H-tetrazol-5- | 355.2 | Compound 71C*: Chiral RT(min): 28.52 |
| | yl)azepan-3-yl)-1-(p-tolyl)cyclopropane-1- | | |
| | carboxamide | | |
| 71D* | N-((3S,5S)-5-methyl-1-(1H-tetrazol-5- | 355.2 | Compound 71D*: Chiral RT(min): 7.163 |
| | yl)azepan-3-yl)-1-(p-tolyl)cyclopropane-1- | | |
| | carboxamide | | |
| Rac-72 | 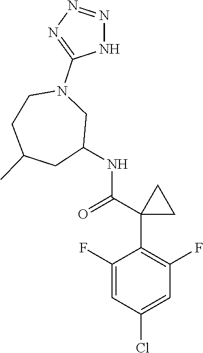 [SMILES: CC1CCN(c2nnn[nH]2)CC(NC(=O)C2(c3c(F)cc(Cl)cc3F)CC2)C1] | 411.2 | Rac-72, 72A*, 72B*, 72C* and 72D* were prepared according to Protocol A and Example 35 using 6-methyl-1-(1H-tetrazol-5-yl)azepan- 3-amine as the amine (i) reagent and 1-(4- chloro-2,6-difluorophenyl)cyclopropane-1- carboxylic acid as the carboxyl (ii) reagent. Rac-72 was then subjected to reverse phase column chromatography (C18 silica gel; mobile phase, Acetonitrile in Water , 0% to 100% gradient in 30 min; detector, UV 254 nm) to provide a mixture of compounds 72B* and 72C* as the first eluting peak, and a mixture of compounds 72A* and 72D* as the second eluting peak. Chiral separation conditions for assumed cis isomers, 72A* and 72D *: CHIRALPAK IG, 2*25 cm, 5 μm; Mobile Phase A: Hexanes (0.1% trifluoroacetic acid), Mobile Phase B: Ethanol; Flow rate: 20 mL/min; Gradient: 10% B to 10% B in 15 min; Wave Length: 220/254 nm. Chiral separation conditions for assumed trans |
| | 1-(4-chloro-2,6-difluorophenyl)-N-(5-methyl-1- | | isomers, 72B* and 72C *: Lux 5 um Cellulose-4, |
| | (1H-tetrazol-5-yl)azepan-3-yl)cyclopropane-1- | | 2.12*25 cm, 5 μm; Mobile Phase A: Hexanes |
| | carboxamide | | (0.1% trifluoroacetic acid), Mobile Phase B: |
| | | | Ethanol;Flow rate: 20 mL/min; Gradient: 30% |
| | | | B to 30% B in 10 min; Wave Length: 220/254 |
| | | | nm. |
| 72A* | 1-(4-chloro-2,6-difluorophenyl)-N-((3R,5R)-5- | 411.2 | Compound 72A*: Chiral RT(min): 12.78 |
| | methyl-1-(1H-tetrazol-5-yl)azepan-3- | | |
| | yl)cyclopropane-1-carboxamide | | |
| 72B* | 1-(4-chloro-2,6-difluorophenyl)-N-((3S,5R)-5- | 411.2 | Compound 72B*: Chiral RT(min): 7.19 |
| | methyl-1-(1H-tetrazol-5-yl)azepan-3- | | |
| | yl)cyclopropane-1-carboxamide | | |
| 72C* | 1-(4-chloro-2,6-difluorophenyl)-N-((3R,5S)-5- | 411.2 | Compound 72C*: Chiral RT(min): 8.38 |
| | methyl-1-(1H-tetrazol-5-yl)azepan-3- | | |
| | yl)cyclopropane-1-carboxamide | | |
| 72D* | 1-(4-chloro-2,6-difluorophenyl)-N-((3S,5S)-5- | 411.2 | Compound 72D*: Chiral RT(min): 9.88 |
| | methyl-1-(1H-tetrazol-5-yl)azepan-3- | | |
| | yl)cyclopropane-1-carboxamide | | |
| Rac-73 | 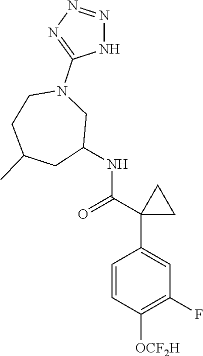 | 425.2 | Rac-73, 73A*, 73B*, 73C*, and 73D* were prepared according to Protocol A and Example 1 using 5-methyl-1-(1H-tetrazol-5-yl)azepan-3- amine as the amine (i) reagent and 1-(3-fluoro- 4-difluoromethoxyphenyl)cyclopropane-1- carboxylic acid as the carboxyl (ii) reagent. Rac-73 was then subjected to reverse phase column chromatography (XBridge Prep Phenyl OBD Column, 19*150 mm, 5 μm; Mobile Phase A: Water (10 mmol/L NH₄HCO₃), Mobile Phase B: acetonitrile; Flow rate: 60 mL/min; Gradient: 20% B to 30% B in 8 min; Wave Length: 220/254 nm) to provide a mixture of compounds 73B* and 73C* as the first eluting peak, and a mixture of compounds 73A* and 73D* as the second eluting peak. Chiral separation conditions for assumed cis isomers, 73A* and 73D *: CHIRALPAK IG, 2*25 cm, 5 μm; Mobile Phase A: Hexanes (0.1% trifluoroacetic acid), Mobile Phase B: isopropanol:dichloromethane =1:1; Flow rate: 20 mL/min; Gradient: 10% B to 10% B in 38 |
| | 1-(4-(difluoromethoxy)-3-fluorophenyl)-N-(5- | | min; Wave Length: 220/254 nm. |
| | methyl-1-(1H-tetrazol-5-yl)azepan-3- | | Chiral separation conditions for assumed trans |
| | yl)cyclopropane-1-carboxamide | | isomers, 73B* and 73C *: CHIRAL ART |
| | | | Cellulose-SB, 2*25 cm, 5 μm; Mobile Phase A: |
| | | | Hexanes (0.1% trifluoroacetic acid), Mobile |
| | | | Phase B: isopropanol:dichloromethane = 1:1; |
| | | | Flow rate: 20 mL/min; Gradient: 15% B to 15% |
| | | | B in 15 min; Wave Length: 220/254 nm. |
| 73A* | 1-(4-(difluoromethoxy)-3-fluorophenyl)-N- | 425.2 | Compound 73A*: Chiral RT(min): 35.57 |
| | ((3R,5R)-5-methyl-1-(1H-tetrazol-5-yl)azepan- | | |
| | 3-yl)cyclopropane-1-carboxamide | | |
| 73B* | 1-(4-(difluoromethoxy)-3-fluorophenyl)-N- | 425.2 | Compound 73B*: Chiral RT(min): 12.39 |
| | ((3S,5R)-5-methyl-1-(1H-tetrazol-5-yl)azepan- | | |
| | 3-yl)cyclopropane-1-carboxamide | | |
| 73C* | 1-(4-(difluoromethoxy)-3-fluorophenyl)-N- | 425.2 | Compound 73C*: Chiral RT(min): 14.18 |
| | ((3R,5S)-5-methyl-1-(1H-tetrazol-5-yl)azepan- | | |
| | 3-yl)cyclopropane-1-carboxamide | | |
| 73D* | 1-(4-(difluoromethoxy)-3-fluorophenyl)-N- | 425.2 | Compound 73D*: Chiral RT(min): 29.71 |
| | ((3S,5S)-5-methyl-1-(1H-tetrazol-5-yl)azepan- | | |
| | 3-yl)cyclopropane-1-carboxamide | | |
| Rac-74 | 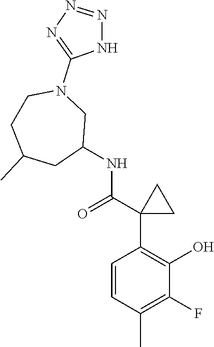 [SMILES: Cc1ccc(C2(C(=O)NC3CC(C)CCN(c4nnn[nH]4)C3)CC2)c(O)c1F] | 389.2 | Rac-74, 74A*, 74B*, 74C*, 74D*, and the corresponding protected —OMe phenol compounds, were prepared according to Protocol A and Example 1 using 5-methyl-1- (1H-tetrazol-5-yl)azepan-3-amine as the amine (i) reagent and 1-(3-fluoro-2-methoxy-4- methylpheny1)cyclopropane-1-carboxylic acid as the carboxyl (ii) reagent. Rac-74 was then subjected to reverse phase column chromatography (XBridge Prep Phenyl OBD Column, 19*250 mm, 5 μm; Mobile Phase A: Water (10 mmol/L NH₄HCO₃), Mobile Phase B: acetonitrile; Flow rate: 20 mL/min; Gradient: 21% B to 23% B in 8 min, 23% B; Wave Length: 254 nm) to provide a mixture of compounds 74B* and 74C* as the first eluting peak, and a mixture of compounds 74A* and 74D* as the second eluting peak. Chiral separation conditions for assumed cis isomers, 74A* and 74D *: Lux 5 um Cellulose-4, 2.12*25 cm, 5 μm; Mobile Phase A: Hexanes |
| | 1-(3-fluoro-2-hydroxy-4-methylphenyl)-N-(5- | | (0.1% trifluoroacetic acid), Mobile Phase B: |
| | methyl-1-(1H-tetrazol-5-yl)azepan-3- | | isopropanol; Flow rate: 20 mL/min; Gradient: |
| | yl)cyclopropane-1-carboxamide | | 30% B to 30% B in 21 min; Wave Length: |
| | | | 220/254 nm. |
| | | | Chiral separation conditions for assumed trans |
| | | | isomers, 74B* and 74C *: CHIRALPAK IE, |
| | | | 2*25 cm, 5 μm; Mobile Phase A: Hexanes |
| | | | (0.1% trifluoroacetic acid), Mobile Phase B: |
| | | | Ethanol:dichloromethane = 1:1; Flow rate: 20 |
| | | | mL/min; Gradient: 30% B to 30% B in 16 min; |
| | | | Wave Length: 220/254 nm. |
| 74A* | 1-(3-fluoro-2-hydroxy-4-methylphenyl)-N- | 389.2 | Compound 74A*: Chiral RT(min): 18.03 |
| | ((3R,5R)-5-methyl-1-(1H-tetrazol-5-yl)azepan- | | |
| | 3-yl)cyclopropane-1-carboxamide | | |
| 74B* | 1-(3-fluoro-2-hydroxy-4-methylphenyl)-N- | 389.2 | Compound 74B*: Chiral RT(min): 9.86 |
| | ((3S,5R)-5-methyl-1-(1H-tetrazol-5-yl)azepan- | | |
| | 3-yl)cyclopropane-1-carboxamide | | |
| 74C* | 1-(3-fluoro-2-hydroxy-4-methylphenyl)-N- | 389.2 | Compound 74C*: Chiral RT(min): 13.97 |
| | ((3R,5S)-5-methyl-1-(1H-tetrazol-5-yl)azepan- | | |
| | 3-yl)cyclopropane-1-carboxamide | | |
| 74D* | 1-(3-fluoro-2-hydroxy-4-methylphenyl)-N- | 389.2 | Compound 74D*: Chiral RT(min): 14.21 |
| | ((3S,5S)-5-methyl-1-(1H-tetrazol-5-yl)azepan- | | |
| | 3-yl)cyclopropane-1-carboxamide | | |
| 75A* | N-((1R,2R,5R)-8-(1H-tetrazol-5-yl)-8- | | Compound 75A*, and the corresponding —OMe |
| | azabicyclo[3.2.1]octan-2-yl)-1-(4-chloro-2- | | protected phenol compound, may be prepared |
| | hydroxyphenyl)cyclopropane-1-carboxamide | | according to Protocol A and Example 34 using |
| | | | (1R,2R,5R)-8-(1H-tetrazol-5-yl)-8- |
| | | | azabicyclo[3.2.1]octan-2-amine as the amine (i) |
| | | | reagent and 1-(4-chloro-2- |
| | | | methoxyphenyl)cyclopropane-1-carboxylic acid |
| | | | as the carboxyl (ii) reagent. |
| 75B* | N-((1R,2S,5R)-8-(1H-tetrazol-5-yl)-8- | 389.2 | Compound 75B* and 76C*, and the |
| | azabicyclo[3.2.1]octan-2-yl)-1-(4-chloro-2- | | corresponding —OMe protected phenol |
| | hydroxyphenyl)cyclopropane-1-carboxamide | | compounds, were prepared according to |
| | | | Protocol A and Example 34 using trans (RSR |
| | | | and SRS) 8-(1H-tetrazol-5-yl)-8- |
| | | | azabicyclo[3.2.1]octan-2-amine as the amine (i) |
| | | | reagent and 1-(4-chloro-2-methoxyphenyl) |
| | | | cyclopropane-1-carboxylic acid as the carboxyl |
| | | | (ii) reagent. |
| | | | The mixture of 75B* and 75C* was then |
| 75C* | N-((1S,2R,5S)-8-(1H-tetrazol-5-yl)-8- | 389.2 | subjected to reverse phase column |
| | azabicyclo[3.2.1]octan-2-yl)-1-(4-chloro-2- | | chromatography (CHIRAL ART Cellulose-SC, |
| | hydroxyphenyl)cyclopropane-1-carboxamide | | 2*25 cm, 5 μm; Mobile Phase A: Hexanes |
| | | | (0.1% trifluoroacetic acid), Mobile Phase B: |
| | | | Ethanol; Flow rate: 20 mL/min; Gradient: 15% |
| | | | B to 15% B in 14 min; Wave Length: 220/254 |
| | | | nm). |
| | | | Compound 75B*: Chiral RT(min): 10.09 |
| | | | Compound 75C*: Chiral RT(min): 12.67 |
| 75D* | N-((1S,2S,5S)-8-(1H-tetrazol-5-yl)-8- | — | Compound 75D*, and the corresponding —OMe |
| | azabicyclo[3.2.1]octan-2-yl)-1-(4-chloro-2- | | protected phenol compound, may be prepared |
| | hydroxyphenyl)cyclopropane-1-carboxamide | | according to Protocol A and Example 34 using |
| | | | (1S,2S,5S)-8-(1H-tetrazol-5-yl)-8- |
| | | | azabicyclo[3.2.1]octan-2-amine as the amine (i) |
| | | | reagent and 1-(4-chloro-2- |
| | | | methoxyphenyl)cyclopropane-1-carboxylic acid |
| | | | as the carboxyl (ii) reagent. |
| 76A* | N-((1R,2R,5R)-8-(1H-tetrazol-5-yl)-8- | — | Compound 76A*, and the corresponding —OMe |
| | azabicyclo[3.2.1]octan-2-yl)-1-(4-chloro-3- | | protected phenol compound, may be prepared |
| | fluoro-2-hydroxyphenyl)cyclopropane-1- | | according to Protocol A and Example 34 using |
| | carboxamide | | (1R,2R,5R)-8-(1H-tetrazol-5-yl)-8- |
| | | | azabicyclo[3.2.1]octan-2-amine as the amine (i) |
| | | | reagent and 1-(4-chloro-3-fluoro-2- |
| | | | methoxyphenyl)cyclopropane-1-carboxylic acid |
| | | | as the carboxyl (ii) reagent |
| 76B* | N-((1R,2S,5R)-8-(1H-tetrazol-5-yl)-8- | 407.1 | Compound 76B* and 77C*, and the |
| | azabicyclo[3.2.1]octan-2-yl)-1-(4-chloro-3- | | corresponding —OMe protected phenol |
| | fluoro-2-hydroxyphenyl)cyclopropane-1- | | compounds, were prepared according to |
| | carboxamide | | Protocol A and Example 34 using trans (RSR |
| | | | and SRS) 8-(1H-tetrazol-5-yl)-8- |
| | | | azabicyclo[3.2.1]octan-2-amine as the amine (i) |
| | | | reagent and 1-(4-chloro-3-fluoro-2- |
| | | | methoxyphenyl)cyclopropane-1-carboxylic |
| 76C* | N-((1S,2R,5S)-8-(1H-tetrazol-5-yl)-8- | 407.1 | acid as the carboxyl (ii) reagent. |
| | azabicyclo[3.2.1]octan-2-yl)-1-(4-chloro-3- | | The mixture of 76B* and 79C* was then |
| | fluoro-2-hydroxyphenyl)cyclopropane-1- | | subjected to reverse phase column |
| | carboxamide | | chromatography (CHIRAL ART Cellulose-SC, |
| | | | 2*25 cm, 5 μm; Mobile Phase A: Hexanes |
| | | | (0.1% trifluoroacetic acid), Mobile Phase B: |
| | | | Ethanol; Flow rate: 20 mL/min; Gradient: 15% |
| | | | B to 15% B in 14 min; Wave Length: 220/254 |
| | | | nm). |
| | | | Compound 76B*: Chiral RT(min): 6.99 |
| | | | Compound 76C*: Chiral RT(min): 10.80 |
| 76D* | N-((1S,2S,5S)-8-(1H-tetrazol-5-yl)-8- | — | Compound 76D*, and the corresponding —OMe |
| | azabicyclo[3.2.1]octan-2-yl)-1-(4-chloro-3- | | protected phenol compound, may be prepared |
| | fluoro-2-hydroxyphenyl)cyclopropane-1- | | according to Protocol A and Example 34 using |
| | carboxamide | | (1S,2S,5S)-8-(1H-tetrazol-5-yl)-8- |
| | | | azabicyclo[3.2.1]octan-2-amine as the amine (i) |
| | | | reagent and 1-(4-chloro-3-fluoro-2- |
| | | | methoxyphenyl)cyclopropane-1-carboxylic acid |
| | | | as the carboxyl (ii) reagent. |
| 77A* | N-((1R,2R,5R)-8-(1H-tetrazol-5-yl)-8- | — | Compound 77A*, and the corresponding —OMe |
| | azabicyclo[3.2.1]octan-2-yl)-1-(4- | | protected phenol compound, may be prepared |
| | (difluoromethoxy)-2- | | according to Protocol A and Example 34 using |
| | hydroxyphenyl)cyclopropane-1-carboxamide | | (1R,2R,5R)-8-(1H-tetrazol-5-yl)-8- |
| | | | azabicyclo[3.2.1]octan-2-amine as the amine (i) |
| | | | reagent and 1-(4-(difluoromethoxy)-2- |
| | | | methoxyphenyl)cyclopropane-1-carboxylic acid |
| | | | as the carboxyl (ii) reagent. |
| 77B* | N-((1R,2S,5R)-8-(1H-tetrazol-5-yl)-8- | 421.1 | Compound 77B* and 77C*, and the |
| | azabicyclo[3.2.1]octan-2-yl)-1-(4- | | corresponding —OMe protected phenol |
| | (difluoromethoxy)-2- | | compounds, were prepared according to |
| | hydroxyphenyl)cyclopropane-1-carboxamide | | Protocol A and Example 34 using trans (RSR |
| | | | and SRS) 8-(1H-tetrazol-5-yl)-8- |
| | | | azabicyclo[3.2.1]octan-2-amine as the amine (i) |
| 77C* | N-((1S,2R,5S)-8-(1H-tetrazol-5-yl)-8- | 421.1 | reagent and 1-(4-difluoromethoxy-2- |
| | azabicyclo[3.2.1]octan-2-yl)-1-(4- | | methoxyphenyl)cyclopropane-1-carboxylic acid |
| | (difluoromethoxy)-2- | | as the carboxyl (ii) reagent. |
| | hydroxyphenyl)cyclopropane-1-carboxamide | | The mixture of 77B* and 77C* was then |
| | | | subjected to reverse phase column |
| | | | chromatography (CHIRALPAK ID, 2*25 cm, 5 |
| | | | μm; Mobile Phase A: Hexanes (0.1% |
| | | | trifluoroacetic acid, Mobile Phase B: |
| | | | isopropanol:dichloromethane = 1:1; Flow rate: |
| | | | 20 mL/min; Gradient: 30% B to 30% B in 10.5 |
| | | | min; Wave Length: 254/220 nm). |
| | | | Compound 77B*: Chiral RT(min): 6.28 |
| | | | Compound 77C*: Chiral RT(min): 9.02 |
| 77D* | N-((1S,2S,5S)-8-(1H-tetrazol-5-yl)-8- | — | Compound 77D*, and the corresponding —OMe |
| | azabicyclo[3.2.1]octan-2-yl)-1-(4- | | protected phenol compound, may be prepared |
| | (difluoromethoxy)-2- | | according to Protocol A and Example 34 using |
| | hydroxyphenyl)cyclopropane-1-carboxamide | | (1S,2S,5S)-8-(1H-tetrazol-5-yl)-8- |
| | | | azabicyclo[3.2.1]octan-2-amine as the amine (i) |
| | | | reagent and 1-(4-(difluoromethoxy)-2- |
| | | | methoxyphenyl)cyclopropane-1-carboxylic acid |
| | | | as the carboxyl (ii) reagent. |
| 78A* | N-((1R,2R,5R)-8-(1H-tetrazol-5-yl)-8- | — | Compound 78A*, and the corresponding —OMe |
| | azabicyclo[3.2.1]octan-2-yl)-1-(2-hydroxy-4- | | protected phenol compound, may be prepared |
| | (trifluoromethoxy)phenyl)cyclopropane-1- | | according to Protocol A and Example 34 using |
| | carboxamide | | (1R,2R,5R)-8-(1H-tetrazol-5-yl)-8- |
| | | | azabicyclo[3.2.1]octan-2-amine as the amine (i) |
| | | | reagent and 1-(4-(trifluoromethoxy)-2- |
| | | | methoxyphenyl)cyclopropane-1-carboxylic acid |
| | | | as the carboxyl (ii) reagent. |
| 78B* | N-((1R,2S,5R)-8-(1H-tetrazol-5-yl)-8- | 439.2 | Compound 78B* and 78C*, and the |
| | azabicyclo[3.2.1]octan-2-yl)-1-(2-hydroxy-4- | | corresponding —OMe protected phenol |
| | (trifluoromethoxy)phenyl)cyclopropane-1- | | compounds, were prepared according to |
| | carboxamide | | Protocol A and Example 34 using trans (RSR |
| | | | and SRS) 8-(1H-tetrazol-5-yl)-8- |
| | | | azabicyclo[3.2.1]octan-2-amine as the amine (i) |
| | | | reagent and 1-(4-(trifluoromethoxy)-2- |
| | | | methoxyphenyl)cyclopropane-1-carboxylic acid |
| 78C* | N-((1S,2R,5S)-8-(1H-tetrazol-5-yl)-8- | 439.2 | as the carboxyl (ii) reagent. |
| | azabicyclo[3.2.1]octan-2-yl)-1-(2-hydroxy-4- | | The mixture of 78B* and 78C* was then |
| | (trifluoromethoxy)phenyl)cyclopropane-1- | | subjected to reverse phase column |
| | carboxamide | | chromatography (CHIRAL ART Cellulose-SC, |
| | | | 2*25 cm, 5 μm; Mobile Phase A: Hexanes (0.1% |
| | | | trifluoroacetic acid), Mobile Phase |
| | | | B: Ethanol:dichloromethane = 1:1; Flow rate: 20 |
| | | | mL/min; Gradient: 15% B to 15% B in 20 min; |
| | | | Wave Length: 254/220 nm. |
| | | | Compound 78B*: Chiral RT(min): 15.10 |
| | | | Compound 78C*: Chiral RT(min): 18.05 |
| 78D* | N-((1S,2S,5S)-8-(1H-tetrazol-5-yl)-8- | | Compound 78D*, and the corresponding —OMe |
| | azabicyclo[3.2.1]octan-2-yl)-1-(2-hydroxy-4- | | protected phenol compound, may be prepared |
| | (trifluoromethoxy)phenyl)cyclopropane-1- | | according to Protocol A and Example 34 using |
| | carboxamide | | (1S,2S,5S)-8-(1H-tetrazol-5-yl)-8- |
| | | | azabicyclo[3.2.1]octan-2-amine as the amine (i) |
| | | | reagent and 1-(4-(trifluoromethoxy)-2- |
| | | | methoxyphenyl)cyclopropane-1-carboxylic acid |
| | | | as the carboxyl (ii) reagent. |
| Rac-79 | 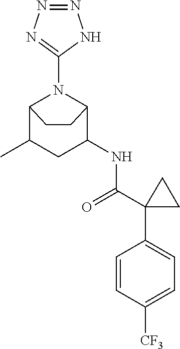 [SMILES: CC1CC(NC(=O)C2(c3ccc(C(F)(F)F)cc3)CC2)C2CCC1N2c1nnn[nH]1] | 421.1 | Rac-79, 79′*, 79″*, 79A*, 79B*, 79C* and 79D* were prepared according to Protocol A and Example 35 using 4-methyl-8-(1H-tetrazol- 5-yl)-8-azabicyclo[3.2.1]octan-2-amine as the amine (i) reagent and 1-(4- (trifluoromethyl)phenyl)cyclopropane-1- carboxylic acid as the carboxyl (ii) reagent. Rac-79 was then subjected to reverse phase column chromatography (C18 silica gel; mobile phase, Acetonitrile in Water, 0% to 100% gradient in 30 min; detector, UV 254 nm) to afford a mixture of compounds 79B* and 79C* as the first eluting peak, and a mixture of compounds 79A* and 79D* as the second eluting peak. Chiral separation conditions for assumed trans isomers, 79B* and 79C *: CHIRALPAK ID 2*25 cm, 5 um; Mobile Phase A: Hexanes (0.1% trifluoroacetic acid), Mobile Phase B: isopropanol; Flow rate: 20 mL/min; Gradient: 20% B to 20% B in 15 min; Wave Length: |
| | N-(4-methy1-8-(1H-tetrazol-5-yl)-8- | | 220/254 nm. |
| | azabicyclo[3.2.1]octan-2-yl)-1-(4- | | Chiral separation conditions for assumed cis |
| | (trifluoromethyl)phenyl)cyclopropane-1- | | isomers, 79A* and 79D*: Lux 5 um Cellulose-4 |
| | carboxamide | | 2.12*25 cm, 5 um; Mobile Phase A: Hexanes |
| 79′* | N-((1R,2R,5S)-4-methyl-8-(1H-tetrazol-5-yl)-8- | 421.1 | (0.1% trifluoroacetic acid), Mobile Phase B: |
| | azabicyclo[3.2.1]octan-2-yl)-1-(4- | | isopropanol; Flow rate: 20 mL/min; Gradient: |
| | (trifluoromethyl)phenyl)cyclopropane-1- | | 30% B to 30% B in 11 min; Wave Length: |
| | carboxamide | | 220/254 nm. |
| 79″* | N-((1S,2S,5R)-4-methyl-8-(1H-tetrazol-5-yl)-8- | 421.1 | Chiral separation conditions for assumed cis |
| | azabicyclo[3.2.1]octan-2-yl)-1-(4- | | isomers, 79A* and 79D*: Lux 5 um Cellulose-4 |
| | (trifluoromethyl)phenyl)cyclopropane-1- | | 2.12*25 cm, 5 um; Mobile Phase A: Hexanes |
| | carboxamide | | (0.1% trifluoroacetic acid), Mobile Phase B: |
| | | | isopropanol; Flow rate: 20 mL/min; Gradient: |
| | | | 30% B to 30% B in 11 min; Wave Length: |
| | | | 220/254 nm. |
| 79A* | N-((1R,2R,4R,5S)-4-methyl-8-(1H-tetrazol-5- | 421.1 | Compound 79A*: Chiral RT(min): 8.61 |
| | yl)-8-azabicyclo[3.2.1]octan-2-yl)-1-(4- | | |
| | (trifluoromethyl)phenyl)cyclopropane-1- | | |
| | carboxamide | | |
| 79B* | N-((1R,2S,4R,5S)-4-methy1-8-(1H-tetrazol-5- | 421.1 | Compound 79B*: Chiral RT(min): 8.13 |
| | yl)-8-azabicyclo[3.2.1]octan-2-yl)-1-(4- | | |
| | (trifluoromethyl)phenyl)cyclopropane-1- | | |
| | carboxamide | | |
| 79C* | N-((1S,2R,4S,5R)-4-methy1-8-(1H-tetrazol-5- | 421.1 | Compound 79C*: Chiral RT(min): 13.13 |
| | yl)-8-azabicyclo[3.2.1]octan-2-yl)-1-(4- | | |
| | (trifluoromethyl)phenyl)cyclopropane-1- | | |
| | carboxamide | | |
| 79D* | N-((1S,2S,4S,5R)-4-methyl-8-(1H-tetrazol-5- | 421.1 | Compound 79D*: Chiral RT(min): 10.89 |
| | yl)-8-azabicyclo[3.2.1]octan-2-yl)-1-(4- | | |
| | (trifluoromethyl)phenyl)cyclopropane-1- | | |
| | carboxamide | | |
| Rac-80 | 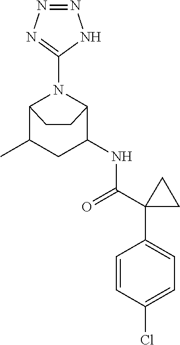 [SMILES: CC1CC(NC(=O)C2(c3ccc(Cl)cc3)CC2)C2CCC1N2c1nnn[nH]1] | 387.1 | Rac-80, 80′*, 80″*, 80A*, 80B*, 80C* and 80D* were prepared according to Protocol A and Example 35 using 4-methy1-8-(1H-tetrazol- 5-yl)-8-azabicyclo[3.2.1]octan-2-amine as the amine (i) reagent and 1-(4- chlorophenyl)cyclopropane-1-carboxylic acid as the carboxyl (ii) reagent. Rac-80 was then subjected to reverse phase column chromatography (C18 silica gel; mobile phase, Acetonitrile in Water, 0% to 100% gradient in 30 min; detector, UV 254 nm) to afford a mixture of compounds 80B* and 80C* as the first eluting peak, and a mixture of compounds 80A* and 80D* as the second eluting peak. Chiral separation conditions for assumed cis isomers, 80A* and 80D *: CHIRALPAK ID 2*25 cm, 5 um; Mobile Phase A: Hexanes (0.1% trifluoroacetic acid), Mobile Phase B: isopropanol:dichloromethane = 1:1; Flow rate: 20 mL/min; Gradient: 30% B to 30% B in 13 |
| | 1-(4-chlorophenyl)-N-(4-methyl-8-(1H-tetrazol- | | min; Wave Length: 220/254 nm. |
| | 5-yl)-8-azabicyclo[3.2.1]octan-2- | | Chiral separation conditions for assumed trans |
| | yl)cyclopropane-1-carboxamide | | isomers, 80A* and 80D *: CHIRALPAK ID |
| 80′* | N-((1R,2R,5S)-4-methyl-8-(1H-tetrazol-5-yl)-8- | 387.1 | 2*25 cm, 5 um; Mobile Phase A: Hexanes (0.1% |
| | azabicyclo[3.2.1]octan-2-yl)-1-(4- | | trifluoroacetic acid), Mobile Phase B: |
| | chlorophenyl)cyclopropane-1-carboxamide | | isopropanol:dichloromethane = 1:1; Flow rate: |
| | | | 20 mL/min; Gradient: 20% B to 20% B in 23 |
| | | | min; Wave Length: 220/254 nm |
| 80″* | N-((1S,2S,5R)-4-methyl-8-(1H-tetrazol-5-yl)-8- | 387.1 | |
| | azabicyclo[3.2.1]octan-2-yl)-1-(4- | | |
| | chlorophenyl)cyclopropane-1-carboxamide | | |
| 80A* | 1-(4-chlorophenyl)-N-((1R,2R,4R,5S)-4-methyl- | 387.1 | Compound 80A*: Chiral RT(min): 9.98 |
| | 8-(1H-tetrazol-5-yl)-8-azabicyclo[3.2.1]octan-2- | | |
| | yl)cyclopropane-1-carboxamide | | |
| 80B* | 1-(4-chlorophenyl)-N-((1R,2S,4R,5S)-4-methyl- | 387.1 | Compound 80B*: Chiral RT(min): 16.38 |
| | 8-(1H-tetrazol-5-yl)-8-azabicyclo[3.2.1]octan-2- | | |
| | yl)cyclopropane-1-carboxamide | | |
| 80C* | 1-(4-chlorophenyl)-N-((1S,2R,4S,5R)-4-methyl- | 387.1 | Compound 80C*: Chiral RT(min): 20.61 |
| | 8-(1H-tetrazol-5-yl)-8-azabicyclo[3.2.1]octan-2- | | |
| | yl)cyclopropane-1-carboxamide | | |
| 80D* | 1-(4-chlorophenyl)-N-((1S,2S,4S,5R)-4-methyl- | 387.1 | Compound 80D*: Chiral RT(min): 5.87 |
| | 8-(1H-tetrazol-5-yl)-8-azabicyclo[3.2.1]octan-2- | | |
| | yl)cyclopropane-1-carboxamide | | |
| Rac-81 | 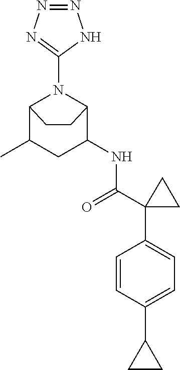 [SMILES: CC1CC(NC(=O)C2(c3ccc(C4CC4)cc3)CC2)C2CCC1N2c1nnn[nH]1] | 393.2 | Rac-81, 81'*, 81″*, 81A*, 81B*, 81C* and 81D* were prepared according to Protocol A and Example 35 using 4-methyl-8-(1H-tetrazol- 5-yl)-8-azabicyclo[3.2.1]octan-2-amine as the amine (i) reagent, and 1-(4- cyclopropylphenyl)cyclopropane-1-carboxylic acid as the carboxyl (ii) reagent. Rac-81 was then subjected to reverse phase column chromatography (Column: XSelect CSH Prep C18 OBD Column, 50*250 mm, 5 μm; Mobile Phase A: Water (0.1% trifluoroacetic acid), Mobile Phase B: acetonitrile; Flow rate: 80 mL/min; Gradient: 50% B to 65% B in 24 min; Wave Length: 254/220 nm) to afford a mixture of compounds 81B* and 81C* as the first eluting peak, and a mixture of compounds 81A* and 81D* as the second eluting peak. Chiral separation conditions for assumed trans isomers, 81B* and 81C *: Column: CHIRALPAK IE, 2*25 cm, 5 μm; Mobile Phase A: Hexanes (0.1% trifluoroacetic acid), Mobile Phase B: Ethanol:dichloromethane = 1:1; Flow |
| | 1-(4-cyclopropylphenyl)-N-(4-methyl-8-(1H- | | rate: 20 mL/min; Gradient: 30% B to 30% B in |
| | tetrazol-5-yl)-8-azabicyclo[3.2.1]octan-2- | | 14 min; Wave Length: 220/254 nm. |
| | yl)cyclopropane-1-carboxamide | | Chiral separation conditions for assumed cis |
| 81′* | N-((1R,2R,5S)-4-methyl-8-(1H-tetrazol-5-yl)-8- | 393.2 | isomers, 81A* and 81D *: Column: |
| | azabicyclo[3.2.1]octan-2-yl)-1-(4-(cyclo- | | CHIRALPAK IC, 2*25 cm, 5 μm; Mobile |
| | propyl)phenyl)cyclopropane-1- | | Phase A: Hexanes (0.1% trifluoroacetic acid), |
| | carboxamide | | Mobile Phase B: Ethanol:dichloromethane = 1:1- |
| 81″* | N-((1S,2S,5R)-4-methyl-8-(1H-tetrazol-5-yl)-8- | 393.2 | -HPLC; Flow rate: 20 mL/min; Gradient: 25% |
| | azabicyclo[3.2.1]octan-2-yl)-1-(4-(cyclo- | | B to 25% B in 15 min; Wave Length: 220/254 |
| | propyl)phenyl)cyclopropane | | nm. |
| 81A* | 1-(4-cyclopropylphenyl)-N-((1R,2R,4R,5S)-4- | 393.2 | Compound 81A*: Chiral RT(min): 8.49 |
| | methy1-8-(1H-tetrazol-5-yl)-8- | | |
| | azabicyclo[3.2.1]octan-2-yl)cyclopropane-1- | | |
| | carboxamide | | |
| 81B* | 1-(4-cyclopropylphenyl)-N-((1R,2S,4R,5S)-4- | 393.2 | Compound 81B*: Chiral RT(min): 12.05 |
| | methyl-8-(1H-tetrazol-5-yl)-8- | | |
| | azabicyclo[3.2.1]octan-2-yl)cyclopropane-1- | | |
| | carboxamide | | |
| 81C* | 1-(4-cyclopropylphenyl)-N-((1S,2R,4S,5R)-4- | 393.2 | Compound 81C*: Chiral RT(min): 8.78 |
| | methyl-8-(1H-tetrazol-5-yl)-8- | | |
| | azabicyclo[3.2.1]octan-2-yl)cyclopropane-1- | | |
| | carboxamide | | |
| 81D* | 1-(4-cyclopropy1-3-fluoro-2-hydroxyphenyl)-N- | 393.2 | Compound 81D*: Chiral RT(min): 11.91 |
| | ((1S,2S,4S,5R)-4-methyl-8-(1H-tetrazol-5-yl)-8- | | |
| | azabicyclo[3.2.1]octan-2-yl)cyclopropane-1- | | |
| | carboxamide | | |
| Rac-82 | 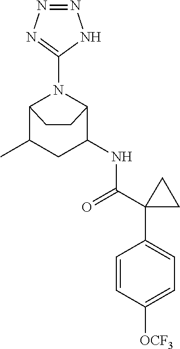 [SMILES: CC1CC(NC(=O)C2(c3ccc(OC(F)(F)F)cc3)CC2)C2CCC1N2c1nnn[nH]1] | 437.2 | Rac-82, 82'*, 82″*, 82A*, 82B*, 82C* and 82D* were prepared according to Protocol A and Example 35 using 4-methyl-8-(1H-tetrazol- 5-yl)-8-azabicyclo[3.2.1]octan-2-amine as the amine (i) reagent and 1-(4- (trifluoromethoxy)phenyl)cyclopropane-1- carboxylic acid as the carboxyl (ii) reagent. Rac-82 was then subjected to reverse phase column chromatography (Column: Xselect CSH C18 OBD Column 30*150 mm; Mobile Phase A: Water(0.05% trifluoroacetic acid), Mobile Phase B: acetonitrile; Flow rate: 60 mL/min mL/min; Gradient: 42% B to 50% B in 10 min; Wave Length: 254 nm/220 nm) to afford a mixture of compounds 82B* and 82C* as the first eluting peak, and a mixture of compounds 82A* and 82D* as the second eluting peak. Chiral separation conditions for assumed trans isomers, 82B* and 82C *: Column: CHIRALPAK IE, 2*25 cm, 5 μm; Mobile Phase A: Hexanes (0.1% trifluoroacetic acid), Mobile |
| | N-(4-methy1-8-(1H-tetrazol-5-yl)-8- | | Phase B: isopropanol:dichloromethane = 1:1; |
| | azabicyclo[3.2.1]octan-2-yl)-1-(4- | | Flow rate: 20 mL/min; Gradient: 35% B to 35% |
| | (trifluoromethoxy)phenyl)cyclopropane-1- | | B in 7 min; Wave Length: 220/254 nm. |
| | carboxamide | | Chiral separation conditions for assumed cis |
| 82′* | N-((1R,2R,5S)-4-methy1-8-(1H-tetrazol-5-yl)-8- | 437.2 | isomers, 82A* and 82D *: Column: |
| | azabicyclo[3.2.1]octan-2-yl)-1-(4-(tri- | | CHIRALPAK IE, 2*25 cm, 5 μum; Mobile Phase |
| | fluoromethoxy)phenyl)cyclopropane-1- | | A: Hexanes (0.1% trifluoroacetic acid), Mobile |
| | carboxamide | | Phase B: isopropanol:dichloromethane = 1:1; |
| 82″* | N-((1S,2S,5R)-4-methy1-8-(1H-tetrazol-5-yl)-8- | 437.2 | Flow rate: 20 mL/min; Gradient: 35% B to 35% |
| | azabicyclo[3.2.1]octan-2-yl)-1-(4-(tri- | | B in 17 min; Wave Length: 220/254 nm. |
| | fluoromethoxy)phenyl)cyclopropane-1- | | |
| | carboxamide | | |
| 82A* | N-((1R,2R,4R,5S)-4-methy1-8-(1H-tetrazol-5- | 437.2 | Compound 82A*: Chiral RT(min): 14.97 |
| | yl)-8-azabicyclo[3.2.1]octan-2-yl)-1-(4- | | |
| | (trifluoromethoxy)phenyl)cyclopropane-1- | | |
| | carboxamide | | |
| 82B* | N-((1R,2S,4R,5S)-4-methyl-8-(1H-tetrazol-5- | 437.2 | Compound 82B*: Chiral RT(min): 4.95 |
| | yl)-8-azabicyclo[3.2.1]octan-2-yl)-1-(4- | | |
| | (trifluoromethoxy)phenyl)cyclopropane-1- | | |
| | carboxamide | | |
| 82C* | N-((1S,2R,4S,5R)-4-methyl-8-(1H-tetrazol-5- | 437.2 | Compound 82C*: Chiral RT(min): 6.34 |
| | yl)-8-azabicyclo[3.2.1]octan-2-yl)-1-(4- | | |
| | (trifluoromethoxy)phenyl)cyclopropane-1- | | |
| | carboxamide | | |
| 82D* | N-((1S,2S,4S,5R)-4-methy1-8-(1H-tetrazol-5- | 437.2 | Compound 82D*: Chiral RT(min): 11.74 |
| | yl)-8-azabicyclo[3.2.1]octan-2-yl)-1-(4- | | |
| | (trifluoromethoxy)phenyl)cyclopropane-1- | | |
| | carboxamide | | |
| Rac-83 | 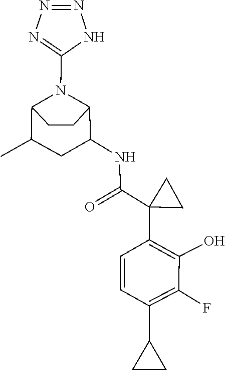 [SMILES: CC1CC(NC(=O)C2(c3ccc(C4CC4)c(F)c3O)CC2)C2CCC1N2c1nnn[nH]1] | 427.2 | Rac-83, 83′*, 83″*, 83A*, 83B*, 83C* and 83D* were prepared according to Protocol A and Example 35 using 4-methy1-8-(1H-tetrazol- 5-yl)-8-azabicyclo[3.2.1]octan-2-amine as the amine (i) reagent and 1-(4-cyclopropyl-3- fluoro-2-hydroxyphenyl)cyclopropane-1- carboxylic acid as the carboxyl (ii) reagent Rac-83 was then subjected to reverse phase column chromatography (Column: Xselect CSH C18 OBD Column 30*150 mm; 5 μm; Mobile Phase A: Water(0.05% trifluoroacetic acid), Mobile Phase B: acetonitrile; Flow rate: 60 mL/min ; Gradient: 29% B to 36% B in 10 min; Wave Length: 254 nm/220 nm) to afford a mixture of compounds 83B* and 83C* as the first eluting peak, and a mixture of compounds 83A* and 83D* as the second eluting peak. Chiral separation conditions for assumed trans isomers, 83B* and 83C *: Column: Chiral ART Cellulose-SA, 2*25 cm, 5 μm; Mobile Phase A: Hexanes (0.1% trifluoroacetic acid), Mobile Phase B: Ethanol:dichloromethane = 1:1; Flow |
| | 1-(4-cyclopropyl-3-fluoro-2-hydroxyphenyl)-N- | | rate: 20 mL/min; Gradient: 15% B to 15% B in |
| | (4-methyl-8-(1H-tetrazol-5-yl)-8- | | 10.5 min; Wave Length: 220/254 nm. |
| | azabicyclo[3.2.1]octan-2-yl)cyclopropane-1- | | Chiral separation conditions for assumed cis |
| | carboxamide | | isomers, 83A* and 83D *: Column: CHIRAL |
| 83′* | N-((1R,2R,5S)-4-methy1-8-(1H-tetrazol-5-yl)-8- | 427.2 | ART Cellulose-SC, 2*25 cm, 5 μm; Mobile |
| | azabicyclo[3.2.1]octan-2-yl)-1-(4-cyclopropyl- | | Phase A: Hexanes (0.1% trifluoroacetic acid), |
| | 3-fluoro-2-hydroxyphenyl)cyclopropane-1 | | Mobile Phase B: Ethanol:dichloromethane = 1:1; |
| 83″* | N-((1S,2S,5R)-4-methyl-8-(1H-tetrazol-5-yl)-8- | 427.2 | Flow rate: 20 mL/min; Gradient: 17% B to 17% |
| | azabicyclo[3.2.1]octan-2-yl)-1-(4-cyclopropyl- | | B in 14.5 min; Wave Length: 220/254 nm. |
| | 3-fluoro-2-hydroxyphenyl)cyclopropane | | |
| 83A* | 1-(4-cyclopropy1-3-fluoro-2-hydroxyphenyl)-N- | 427.2 | Compound 83A*: Chiral RT(min): 11.78 |
| | ((1R,2R,4R,5S)-4-methy1-8-(1H-tetrazol-5-yl)- | | |
| | 8-azabicyclo[3.2.1]octan-2-yl)cyclopropane-1- | | |
| | carboxamide | | |
| 83B* | 1-(4-cyclopropy1-3-fluoro-2-hydroxyphenyl)-N- | 427.2 | Compound 83B*: Chiral RT(min): 6.95 |
| | ((1R,2S,4R,5S)-4-methyl-8-(1H-tetrazol-5-yl)- | | |
| | 8-azabicyclo[3.2.1]octan-2-yl)cyclopropane-1- | | |
| | carboxamide | | |
| 83C* | 1-(4-cyclopropy1-3-fluoro-2-hydroxyphenyl)-N- | 427.2 | Compound 83C*: Chiral RT(min): 9.96 |
| | ((1S,2R,4S,5R)-4-methy1-8-(1H-tetrazol-5-yl)- | | |
| | 8-azabicyclo[3.2.1]octan-2-yl)cyclopropane-1- | | |
| | carboxamide | | |
| 83D* | 1-(4-cyclopropyl-3-fluoro-2-hydroxyphenyl)-N- | 427.2 | Compound 83D*: Chiral RT(min): 14.73 |
| | ((1S,2S,4S,5R)-4-methy1-8-(1H-tetrazol-5-yl)-8- | | |
| | azabicyclo[3.2.1]octan-2-yl)cyclopropane-1- | | |
| | carboxamide | | |
| Rac-84 | 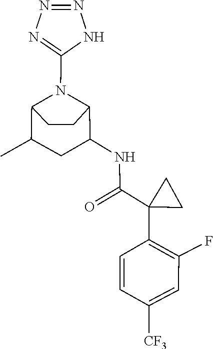 [SMILES: CC1CC(NC(=O)C2(c3ccc(C(F)(F)F)cc3F)CC2)C2CCC1N2c1nnn[nH]1] | 439.2 | Rac-84, 84′*, 84″*, 84A*, 84B*, 84C* and 84D* were prepared according to Protocol A and Example 35 using 4-methyl-8-(1H-tetrazol- 5-yl)-8-azabicyclo[3.2.1]octan-2-amine as the amine (i) reagent and 1-(2-fluoro-4- (trifluoromethyl)phenyl)cyclopropane-1- carboxylic acid as the carboxyl (ii) reagent. Rac-84 was then subjected to reverse phase column chromatography (C18 silica gel; mobile phase, Acetonitrile in Water , 0% to 100% gradient in 30 min; detector, UV 254 nm) to afford a mixture of compounds 84B* and 84C* as the first eluting peak, and a mixture of compounds 84A* and 84D* as the second eluting peak. Chiral separation conditions for assumed trans isomers, 84B* and 84C *: Column: CHIRALPAK IE, 2*25 cm, 5 μm; Mobile Phase A: Hexanes (0.1% trifluoroacetic acid), Mobile Phase B: Ethanol:dichloromethane = 1:1; Flow rate: 20 mL/min; Gradient: 20% B to 20% B in 18 min; Wave Length: 220/254 nm. |
| | 1-(2-fluoro-4-(trifluoromethyl)phenyl)-N-(4- | | Chiral separation conditions for assumed cis |
| | methy1-8-(1H-tetrazol-5-yl)-8- | | isomers, 84A* and 84D *: Column: CHIRAL |
| | azabicyclo[3.2.1]octan-2-yl)cyclopropane-1- | | ART Cellulose-SC, 2*25 cm, 5 μm; Mobile |
| | carboxamide | | Phase A: Hexanes (0.1% trifluoroacetic acid), |
| 84′* | N-((1R,2R,5S)-4-methy1-8-(1H-tetrazol-5-yl)-8- | 439.2 | Mobile Phase B: Ethanol:dichloromethane = 1:1; |
| | azabicyclo[3.2.1]octan-2-yl)-1-(2-fluoro-4- | | Flow rate: 20 mL/min; Gradient: 20% B to 20% |
| | (trifluoromethyl)phenyl)cyclopropane-1- | | B in 15 min; Wave Length: 220/254 nm. |
| | carboxamide yl)cyclopropane-1-carboxamide | | |
| 84″* | N-((1S,2S,5R)-4-methy1-8-(1H-tetrazol-5-yl)-8- | 439.2 | |
| | azabicyclo[3.2.1]octan-2-yl)-1-(2-fluoro-4- | | |
| | (trifluoromethyl)phenyl)cyclopropane-1- | | |
| | carboxamide | | |
| 84A* | 1-(2-fluoro-4-(trifluoromethyl)phenyl)-N- | 439.2 | Compound 84A*: Chiral RT(min): 9.7 |
| | ((1R,2R,4R,5S)-4-methyl-8-(1H-tetrazol-5-yl)- | | |
| | 8-azabicyclo[3.2.1]octan-2-yl)cyclopropane-1- | | |
| | carboxamide | | |
| 84B* | 1-(2-fluoro-4-(trifluoromethyl)phenyl)-N- | 439.2 | Compound 84B*: Chiral RT(min): 11.23 |
| | ((1R,2S,4R,5S)-4-methyl-8-(1H-tetrazol-5-yl)- | | |
| | 8-azabicyclo[3.2.1]octan-2-yl)cyclopropane-1- | | |
| | carboxamide | | |
| 84C* | 1-(2-fluoro-4-(trifluoromethyl)phenyl)-N- | 439.2 | Compound 84C*: Chiral RT(min): 15.44 |
| | ((1S,2R,4S,5R)-4-methy1-8-(1H-tetrazol-5-yl)- | | |
| | 8-azabicyclo[3.2.1]octan-2-yl)cyclopropane-1- | | |
| | carboxamide | | |
| 84D* | 1-(2-fluoro-4-(trifluoromethyl)phenyl)-N- | 439.2 | Compound 84D*: Chiral RT(min): 12.65 |
| | ((1S,2S,4S,5R)-4-methy1-8-(1H-tetrazol-5-yl)-8- | | |
| | azabicyclo[3.2.1]octan-2-yl)cyclopropane-1- | | |
| | carboxamide | | |
| Rac-85 | 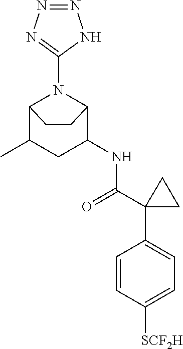 | 435.2 | Rac-85, 85′*, 85″*, 85A*, 85B*, 85C* and 85D* were prepared according to Protocol A and Example 35 using 4-methyl-8-(1H-tetrazol- 5-yl)-8-azabicyclo[3.2.1]octan-2-amine as the amine (i) reagent and 1-(4- ((difluoromethyl)thio)phenyl)cyclopropane-1- carboxylic acid as the carboxyl (ii) reagent. Rac-85 was then subjected to reverse phase column chromatography (C18 silica gel; mobile phase, Acetonitrile in Water, 0% to 100% gradient in 30 min; detector, UV 254 nm) to afford a mixture of compounds 85B* and 85C* as the first eluting peak, and a mixture of compounds 85A* and 85D* as the second eluting peak. Chiral separation conditions for assumed trans isomers, 85B* and 85C *: Column: CHIRALPAK ID, 2*25 cm, 5 μm; Mobile Phase A: Hexanes (0.1% trifluoroacetic acid), Mobile Phase B: Ethanol:dichloromethane = 1:1; Flow rate: 20 mL/min; Gradient: 20% B to 20% |
| | 1-(4-((difluoromethyl)thio)phenyl)-N-(4-methyl- | | B in 12 min; Wave Length: 220/254 nm. |
| | 8-(1H-tetrazol-5-yl)-8-azabicyclo[3.2.1]octan-2- | | Chiral separation conditions for assumed cis |
| | yl)cyclopropane-1-carboxamide | | isomers, 85A* and 85D *: Column: CHIRAL |
| 85′* | N-((1R,2R,5S)-4-methy1-8-(1H-tetrazol-5-yl)-8- | 435.2 | ART Cellulose-SC, 2*25 cm, 5 μm; Mobile |
| | azabicyclo[3.2.1]octan-2-yl)-1-(4- | | Phase A: Hexanes (0.1% trifluoroacetic acid), |
| | ((difluoromethyl)thio)phenyl)cyclopropane-1- | | Mobile Phase B: Ethanol:dichloromethane = 1:1; |
| | carboxamide | | Flow rate: 20 mL/min; Gradient: 30% B to 30% |
| 85″* | N-((1S,2S,5R)-4-methyl-8-(1H-tetrazol-5-yl)-8- | 435.2 | B in 10.5 min; Wave Length: 220/254 nm. |
| | azabicyclo[3.2.1]octan-2-yl)-1-(4- | | |
| | ((difluoromethyl)thio)phenyl)cyclopropane-1- | | |
| | carboxamide | | |
| 85A* | 1-(4-((difluoromethyl)thio)phenyl)-N- | 435.2 | Compound 85A *: Chiral RT(min): 7.22 |
| | ((1R,2R,4R,5S)-4-methyl-8-(1H-tetrazol-5-yl)- | | |
| | 8-azabicyclo[3.2.1]octan-2-yl)cyclopropane-1- | | |
| | carboxamide | | |
| 85B* | 1-(4-((difluoromethyl)thio)phenyl)-N- | 435.2 | Compound 85B *: Chiral RT(min): 8.01 |
| | ((1R,2S,4R,5S)-4-methyl-8-(1H-tetrazol-5-yl)- | | |
| | 8-azabicyclo[3.2.1]octan-2-yl)cyclopropane-1- | | |
| | carboxamide | | |
| 85C* | 1-(4-((difluoromethyl)thio)phenyl)-N- | 435.2 | Compound 85C *: Chiral RT(min): 10.91 |
| | ((1S,2R,4S,5R)-4-methyl-8-(1H-tetrazol-5-yl)- | | |
| | 8-azabicyclo[3.2.1]octan-2-yl)cyclopropane-1- | | |
| | carboxamide | | |
| 85D* | 1-(4-((difluoromethyl)thio)phenyl)-N- | 435.2 | Compound 85D *: Chiral RT(min): 9.03 |
| | ((1S,2S,4S,5R)-4-methy1-8-(1H-tetrazol-5-yl)-8- | | |
| | azabicyclo[3.2.1]octan-2-yl)cyclopropane-1- | | |
| | carboxamide | | |
| Rac-86 | 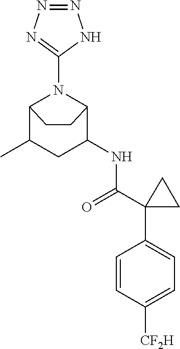 [SMILES: CC1CC(NC(=O)C2(c3ccc(C(F)F)cc3)CC2)C2CCC1N2c1nnn[nH]1] | 403.2 | Rac-86, 86′*, 86″*, 86A*, 86B*, 86C* and 86D* were prepared according to Protocol A and Example 35 using 4-methyl-8-(1H-tetrazol- 5-yl)-8-azabicyclo[3.2.1]octan-2-amine as the amine (i) reagent and 1-(4- (difluoromethyl)phenyl)cyclopropane-1- carboxylic acid as the carboxyl (ii) reagent. Rac-86 was then subjected to reverse phase column chromatography (Column: Xselect CSH OBD Column 30*150 mm, 5 umn; Mobile Phase A: Water (0.05% trifluoroacetic acid), Mobile Phase B: acetonitrile; Flow rate: 20 mL/min; Gradient: 5% B to 5% B in 2 min, 9% B to 18% B in 10 min; Wave Length: 254 nm/220 nm) to afford a mixture of compounds 86B* and 86C* as the first eluting peak, and a mixture of compounds 86A* and 86D* as the second eluting peak. Chiral separation conditions for assumed trans isomers, 86B* and 86C *: Column: CHIRALPAK IE, 2*25 cm, 5 μm; Mobile Phase |
| | 1-(4-(difluoromethyl)phenyl)-N-(4-methyl-8- | | A: Hexanes (0.1% trifluoroacetic acid), Mobile |
| | (1H-tetrazol-5-y1)-8-azabicyclo[3.2.1]octan-2- | | Phase B: Ethanol:dichloromethane = 1:1; Flow |
| | yl)cyclopropane-1-carboxamide | | rate: 20 mL/min; Gradient: 30% B to 30% B in |
| 86'* | N-((1R,2R,5S)-4-methy1-8-(1H-tetrazol-5-yl)-8- | 403.2 | 12 min; Wave Length: 220/254 nm. |
| | azabicyclo[3.2.1]octan-2-yl)-1-(4- | | Chiral separation conditions for assumed cis |
| | (difluoromethyl)phenyl)cyclopropane | | isomers, 86A* and 86D *: Column: CHIRAL |
| 86″* | N-((1S,2S,5R)-4-methyl-8-(1H-tetrazol-5-yl)-8- | 403.2 | ART Cellulose-SC, 2*25 cm, 5 μm; Mobile |
| | azabicyclo[3.2.1]octan-2-yl)-1-(4- | | Phase A: Hexanes (0.1% trifluoroacetic acid), |
| | (difluoromethyl)phenyl)cyclopropane-1- | | Mobile Phase B: Ethanol:dichloromethane=1:1- |
| | carboxamide | | -HPLC; Flow rate: 20 mL/min; Gradient: |
| | | | isocratic ; Wave Length: 220/254 nm. |
| 86A* | 1-(4-(difluoromethyl)phenyl)-N- | 403.2 | Compound 86A*: Chiral RT(min): 7.2 |
| | ((1R,2R,4R,5S)-4-methy1-8-(1H-tetrazol-5-yl)- | | |
| | 8-azabicyclo[3.2.1]octan-2-yl)cyclopropane-1- | | |
| | carboxamide | | |
| 86B* | 1-(4-(difluoromethyl)phenyl)-N- | 403.2 | Compound 86B*: Chiral RT(min): 7.92 |
| | ((1R,2S,4R,5S)-4-methyl-8-(1H-tetrazol-5-yl)- | | |
| | 8-azabicyclo[3.2.1]octan-2-yl)cyclopropane-1- | | |
| | carboxamide | | |
| 86C* | 1-(4-(difluoromethyl)phenyl)-N- | 403.2 | Compound 86C*: Chiral RT(min): 10.71 |
| | ((1S,2R,4S,5R)-4-methyl-8-(1H-tetrazol-5-yl)- | | |
| | 8-azabicyclo[3.2.1]octan-2-yl)cyclopropane-1- | | |
| | carboxamide | | |
| 86D* | 1-(4-(difluoromethyl)phenyl)-N-((1S,2S,4S,5R)- | 403.2 | Compound 86D*: Chiral RT(min): 10.89 |
| | 4-methyl-8-(1H-tetrazol-5-yl)-8- | | |
| | azabicyclo[3.2.1]octan-2-yl)cyclopropane-1- | | |
| | carboxamide | | |
| Rac-87 | 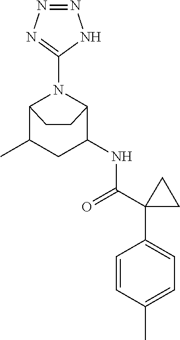 [SMILES: Cc1ccc(C2(C(=O)NC3CC(C)C4CCC3N4c3nnn[nH]3)CC2)cc1] | 365.1 | Rac-87, 87′*, 87″*, 87A*, 87B*, 87C* and 87D* were prepared according to Protocol A and Example 35 using 4-methyl-8-(1H-tetrazol- 5-yl)-8-azabicyclo[3.2.1]octan-2-amine as the amine (i) reagent and 1-(p-toly1)cyclopropane- 1-carboxylic acid as the carboxyl (ii) reagent. Rac-87 was then subjected to reverse phase column chromatography (C18 silica gel; mobile phase, Acetonitrile in Water, 0% to 100% gradient in 30 min; detector, UV 254 nm) to afford a mixture of compounds 87B* and 87C* as the first eluting peak, and a mixture of compounds 87A* and 87D* as the second eluting peak. Chiral separation conditions for assumed trans isomers, 87B* and 87C *: Column: CHIRALPAK IH, 2*25 cm, 5 μm; Mobile Phase A: Hexanes (0.1% trifluoroacetic acid), Mobile Phase B: Ethanol; Flow rate: 20 mL/min; Gradient: 15% B to 15% B in 11.5 min; Wave Length: 220/254 nm. |
| | N-(4-methy1-8-(1H-tetrazol-5-yl)-8- | | Chiral separation conditions for assumed cis |
| | azabicyclo[3.2.1]octan-2-yl)-1-(p- | | isomers, 87A* and 87D *: Column: Lux 5 um |
| | tolyl)cyclopropane-1-carboxamide | | Cellulose-4, 2.12*25 cm, 5 μm; Mobile Phase |
| 87′* | N-((1R,2R,5S)-4-methy1-8-(1H-tetrazol-5-yl)-8- | 365.1 | A: Hexanes (0.1% trifluoroacetic acid), Mobile |
| | azabicyclo[3.2.1]octan-2-yl)-1-(4- | | Phase B: Ethanol; Flow rate: 20 mL/min; |
| | tolyl)cyclopropane-1-carboxamide | | Gradient: 30% B to 30% B in 10.2 min; Wave |
| | | | Length: 220/254 nm. |
| 87″* | N-((1S,2S,5R)-4-methy1-8-(1H-tetrazol-5-yl)-8- | 365.1 | |
| | azabicyclo[3.2.1]octan-2-yl)-1-(4- | | |
| | tolyl)cyclopropane-1-carboxamide | | |
| 87A* | N-((1R,2R,4R,5S)-4-methy1-8-(1H-tetrazol-5- | 365.1 | Compound 87A*: Chiral RT(min): 7.76 |
| | yl)-8-azabicyclo[3.2.1]octan-2-yl)-1-(p- | | |
| | tolyl)cyclopropane-1-carboxamide | | |
| 87B* | N-((1R,2S,4R,5S)-4-methy1-8-(1H-tetrazol-5- | 365.1 | Compound 87B*: Chiral RT(min): 7.08 |
| | yl)-8-azabicyclo[3.2.1]octan-2-yl)-1-(p- | | |
| | tolyl)cyclopropane-1-carboxamide | | |
| 87C* | N-((1S,2R,4S,5R)-4-methy1-8-(1H-tetrazol-5- | 365.1 | Compound 87C*: Chiral RT(min): 10.37 |
| | yl)-8-azabicyclo[3.2.1]octan-2-yl)-1-(p- | | |
| | tolyl)cyclopropane-1-carboxamide | | |
| 87D* | N-((1S,2S,4S,5R)-4-methy1-8-(1H-tetrazol-5- | 365.1 | Compound 87D*: Chiral RT(min): 9.1 |
| | yl)-8-azabicyclo[3.2.1]octan-2-yl)-1-(p- | | |
| | tolyl)cyclopropane-1-carboxamide | | |
| Rac-88 | 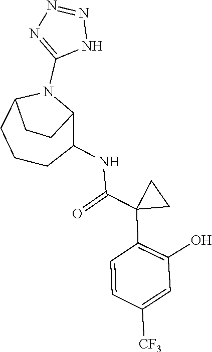 [SMILES: O=C(NC1CCCC2CCC1N2c1nnn[nH]1)C1(c2ccc(C(F)(F)F)cc2O)CC1] | 437.2 | Compound Rac-88, 88A*, 88B*, 88C* and 88D*, and the corresponding protected —OMe phenol compounds, were prepared according to Protocol C and Example 9 using tert-buty1 2- amino-9-azabicyclo[4.2.1]nonane-9-carboxylate as the protected amine (iv) reagent and 1-(2- hydroxy-4- (trifluoromethyl)phenyl)cyclopropane-1- carboxylic acid as the carboxyl (ii) reagent. Rac-88 was then subjected to reverse phase column chromatography (C18 silica gel; mobile phase, Acetonitrile in Water , 0% to 100% gradient in 30 min; detector, UV 254 nm) to provide a mixture of compounds 88B* and 88C* as the first eluting peak, and a mixture of compounds 88A* and 88D* as the second eluting peak. Chiral separation conditions for assumed cis isomers, 88A* and 88D*: Lux 5 um Cellulose-4, 2.12*25 cm, 5 μm; Mobile Phase A: Hexanes (0.1% trifluoroacetic acid), Mobile Phase B: isopropanol; Flow rate: 20 mL/min; Gradient: |
| | azabicyclo[4.2.1]nonan-2-yl)-1-(2-hydroxy-4- | | 20% B to 20% B in 18 min; Wave Length: |
| | (trifluoromethyl)phenyl)cyclopropane-1- | | 220/254 nm. |
| | carboxamide | | Chiral separation conditions for assumed trans |
| | | | isomers, 88B* and 88C*: CHIRALPAK ID, |
| | | | 2*25 cm, 5 μm; Mobile Phase A: Hexanes |
| | | | (0.1% trifluoroacetic acid), Mobile Phase B: |
| | | | Ethanol:dichloromethane = 1:1; Flow rate: 20 |
| | | | mL/min; Gradient: 13% B to 13% B in 12 min; |
| | | | Wave Length: 220/254 nm. |
| 88A* | N-((1R,2R,6R)-9-(1H-tetrazol-5-yl)-9- | 437.2 | Compound 88A*: Chiral RT(min): 10.06 |
| | azabicyclo[4.2.1]nonan-2-yl)-1-(2-hydroxy-4- | | |
| | (trifluoromethyl)phenyl)cyclopropane-1- | | |
| | carboxamide | | |
| 88B* | N-((1R,2S,6R)-9-(1H-tetrazol-5-yl)-9- | 437.2 | Compound 88B*: Chiral RT(min): 15.78 |
| | azabicyclo[4.2.1]nonan-2-yl)-1-(2-hydroxy-4- | | |
| | (trifluoromethyl)phenyl)cyclopropane-1- | | |
| | carboxamide | | |
| 88C* | N-((1S,2R,6S)-9-(1H-tetrazol-5-yl)-9- | 437.2 | Compound 88C*: Chiral RT(min): 12.45 |
| | azabicyclo[4.2.1]nonan-2-yl)-1-(2-hydroxy-4- | | |
| | (trifluoromethyl)phenyl)cyclopropane-1- | | |
| | carboxamide | | |
| 88D* | N-((1S,2S,6S)-9-(1H-tetrazol-5-yl)-9- | 437.2 | Compound 88D*: Chiral RT(min): 9.31 |
| | azabicyclo[4.2.1]nonan-2-yl)-1-(2-hydroxy-4- | | |
| | (trifluoromethyl)phenyl)cyclopropane-1- | | |
| | carboxamide | | |
| Rac-89 | 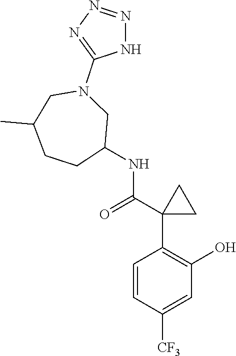 [SMILES: CC1CCC(NC(=O)C2(c3ccc(C(F)(F)F)cc3O)CC2)CN(c2nnn[nH]2)C1] | 425.1 | Compound Rac-89, 89A*, 89B*, 89C* and 89D*, and the corresponding protected —OMe phenol compounds, were prepared according to Protocol A and Example 35 using 6-methyl-1- (1H-tetrazol-5-yl)azepan-3-amine as the amine (i) reagent and 1-(2-hydroxy-4- (trifluoromethyl)phenyl)cyclopropane-1- carboxylic acid as the carboxyl (ii) reagent. Rac-89 was then subjected to reverse phase column chromatography (C18 silica gel; mobile phase, Acetonitrile in Water , 0% to 100% gradient in 30 min; detector, UV 254 nm) to provide a mixture of compounds 89A* and 89D* as the first eluting peak, and a mixture of compounds 89B* and 89C* as the second eluting peak. Chiral separation conditions for assumed cis isomers, 89A* and 89D *: Lux 5 um Cellulose-4, 2.12*25 cm, 5 um Mobile Phase A: Hexanes (0.1% trifluoroacetic acid), Mobile Phase B: Ethanol; Flow rate: 20 mL/min; Gradient: 20% B to 20% B in 6 min; Wave Length: 220/254 |
| | 1-(2-hydroxy-4-(trifluoromethyl)phenyl)-N-(6- | | nm. |
| | methyl-1-(1H-tetrazol-5-yl)azepan-3- | | Chiral separation conditions for assumed trans |
| | yl)cyclopropane-1-carboxamide | | isomers, 89B* and 89C *: Lux 5 um Cellulose-4, |
| | | | 2.12*25 cm, 5 um; Mobile Phase A: Hexanes |
| | | | (0.1% trifluoroacetic acid), Mobile Phase B: |
| | | | Ethanol; Flow rate: 20 mL/min; Gradient: 10% |
| | | | B to 10% B in 9 min; Wave Length: 220/254 |
| | | | nm. |
| 89A* | 1-(2-hydroxy-4-(trifluoromethyl)phenyl)-N- | 425.1 | Compound 89A*: Chiral RT(min): 7.56 |
| | ((3R,6S)-6-methyl-1-(1H-tetrazol-5-yl)azepan- | | |
| | 3-yl)cyclopropane-1-carboxamide | | |
| | 1-(2-hydroxy-4-(trifluoromethyl)phenyl)-N- | 425.1 | Compound 89B*: Chiral RT(min): 16.44 |
| 89B* | ((3S,6S)-6-methyl-1-(1H-tetrazol-5-yl)azepan- | | |
| | 3-yl)cyclopropane-1-carboxamide | | |
| 89C* | 1-(2-hydroxy-4-(trifluoromethyl)phenyl)-N- | 425.1 | Compound 89C*: Chiral RT(min): 17.77 |
| | ((3R,6R)-6-methyl-1-(1H-tetrazol-5-yl)azepan- | | |
| | 3-yl)cyclopropane-1-carboxamide | | |
| 89D* | 1-(2-hydroxy-4-(trifluoromethyl)phenyl)-N- | 425.1 | Compound 89D*: Chiral RT(min): 6.77 |
| | ((3S,6R)-6-methyl-1-(1H-tetrazol-5-yl)azepan- | | |
| | 3-yl)cyclopropane-1-carboxamide | | |
| Rac-90 | 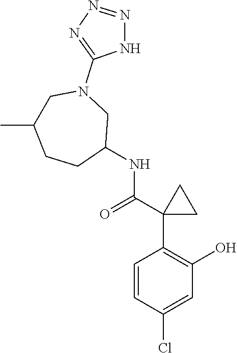 [SMILES: CC1CCC(NC(=O)C2(c3ccc(Cl)cc3O)CC2)CN(c2nnn[nH]2)C1] | 391.1 | Compound Rac-90, 90A*, 90B*, 90C* and 90D*, and the corresponding protected —OMe phenol compounds, were prepared according to Protocol A and Example 35 using 6-methyl-1- (1H-tetrazol-5-yl)azepan-3-amine as the amine (i) reagent and 1-(2-hydroxy-4- chlorophenyl)cyclopropane-1-carboxylic acid as the carboxyl (ii) reagent. Rac-90 was then subjected to reverse phase column chromatography (Column, XBridge Prep Phenyl OBD Column, 19*250 mm, 5 μm; mobile phase, Water (10 mmol/L NH₄HCO₃) and MeOH (45% MeOH up to 60% in 10 min); Wave Length: 220/254 nm) to provide a mixture of compounds 90B* and 90C* as the first eluting peak, and a mixture of compounds 90A* and 90D* as the second eluting peak. Chiral separation conditions for assumed cis isomers, 90A* and 90D *: Lux 5 um Cellulose-4, 2.12*25 cm, 5 um; Mobile Phase A: Hexanes (0.1% trifluoroacetic acid), Mobile Phase B: isopropanol; Flow rate: 20 mL/min; Gradient: |
| | 1-(4-chloro-2-hydroxyphenyl)-N-(6-methyl-1- | | 30% B to 30% B in 13 min; Wave Length: |
| | (1H-tetrazol-5-yl)azepan-3-yl)cyclopropane-1- | | 220/254 nm. |
| | carboxamide | | Chiral separation conditions for assumed trans |
| | | | isomers, 90B* and 90C *: Lux 5 um Cellulose-4, |
| | | | 2.12*25 cm, 5 μm; Mobile Phase A: Hexanes |
| | | | (0.1% trifluoroacetic acid), Mobile Phase B: |
| | | | isopropanol; Flow rate: 20 mL/min; Gradient: |
| | | | 30% B to 30% B in 15 min; Wave Length: |
| | | | 220/254 nm. |
| 90A* | 1-(4-chloro-2-hydroxyphenyl)-N-((3R,6S)-6- | 391.1 | Compound 90A*: Chiral RT(min): 10.7 |
| | methyl-1-(1H-tetrazol-5-yl)azepan-3- | | |
| | yl)cyclopropane-1-carboxamide | | |
| 90BY | 1-(4-chloro-2-hydroxyphenyl)-N-((3S,6S)-6- | 391.1 | Compound 90B*: Chiral RT(min): 8.34 |
| | methy1-1-(1H-tetrazol-5-yl)azepan-3- | | |
| | yl)cyclopropane-1-carboxamide | | |
| 90C* | 1-(4-chloro-2-hydroxyphenyl)-N-((3R,6R)-6- | 391.1 | Compound 90C*: Chiral RT(min): 10.85 |
| | methyl-1-(1H-tetrazol-5-yl)azepan-3- | | |
| | yl)cyclopropane-1-carboxamide | | |
| 90D* | 1-(4-chloro-2-hydroxyphenyl)-N-((3S,6R)-6- | 391.1 | Compound 90D*: Chiral RT(min): 8.62 |
| | methyl-1-(1H-tetrazol-5-yl)azepan-3- | | |
| | yl)cyclopropane-1-carboxamide | | |
| Rac-91 | 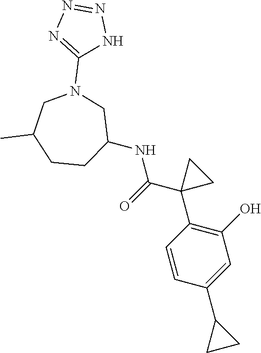 [SMILES: CC1CCC(NC(=O)C2(c3ccc(C4CC4)cc3O)CC2)CN(c2nnn[nH]2)C1] | 397.2 | Compound Rac-91, 91A*, 91B*, 91C* and 91D*, and the corresponding protected —OMe phenol compounds, were prepared according to Protocol A and Example 35 using 6-methyl-1- (1H-tetrazol-5-yl)azepan-3-amine as the amine (i) reagent and 1-(4-cyclopropyl-2- hydroxyphenyl)cyclopropane-1-carboxylic acid as the carboxyl (ii) reagent. Rac-91 was then subjected to reverse phase column chromatography (C18 silica gel; mobile phase, Acetonitrile in Water , 0% to 100% gradient in 30 min; detector, UV 254 nm) to provide a mixture of compounds 91B* and 91C* as the first eluting peak, and a mixture of compounds 91A* and 911D* as the second eluting peak. Chiral separation conditions for assumed cis isomers, 91A* and 91D *: Lux 5 um Cellulose-4 2.12*25 cm, 5 um; Mobile Phase A: Hexanes (0.1% trifluoroacetic acid), Mobile Phase B: Ethanol; Flow rate: 20 mL/min; Gradient: 20% B to 20% B in 21 min; Wave Length: 220/254 |
| | 1-(4-cyclopropy1-2-hydroxyphenyl)-N-(6- | | nm. |
| | methyl-1-(1H-tetrazol-5-yl)azepan-3- | | Chiral separation conditions for assumed trans |
| | yl)cyclopropane-1-carboxamide | | isomers, 91B* and 91C *: Lux 5 um Cellulose-4 |
| | | | 2.12*25 cm, 5 um; Mobile Phase A: Hexanes |
| | | | (0.1% trifluoroacetic acid), Mobile Phase B: |
| | | | Methanol:ethanol = 1:1; Flow rate: 20 |
| | | | mL/min; Gradient: 10% B to 10% B in 30 min; |
| | | | Wave Length: 220/254 nm |
| 91A* | 1-(4-cyclopropy1-2-hydroxyphenyl)-N-((3R,6S)- | 397.2 | Compound 91A*: Chiral RT(min): 18.83 |
| | 6-methyl-1-(1H-tetrazol-5-yl)azepan-3- | | |
| | yl)cyclopropane-1-carboxamide | | |
| 91B* | 1-(4-cyclopropy1-2-hydroxyphenyl)-N-((3S,6S)- | 397.2 | Compound 91B*: Chiral RT(min): 12.13 |
| | 6-methyl-1-(1H-tetrazol-5-yl)azepan-3- | | |
| | yl)cyclopropane-1-carboxamide | | |
| 91C* | 1-(4-cyclopropyl-2-hydroxyphenyl)-N- | 397.2 | Compound 91C*: Chiral RT(min): 12.13 |
| | ((3R,6R)-6-methyl-1-(1H-tetrazol-5-yl)azepan- | | |
| | 3-yl)cyclopropane-1-carboxamide | | |
| 91D* | 1-(4-cyclopropy1-2-hydroxyphenyl)-N-((3S,6R)- | 397.2 | Compound 91D*: Chiral RT(min): 12.13 |
| | 6-methyl-1-(1H-tetrazol-5-yl)azepan-3- | | |
| | yl)cyclopropane-1-carboxamide | | |
| Rac-92 | 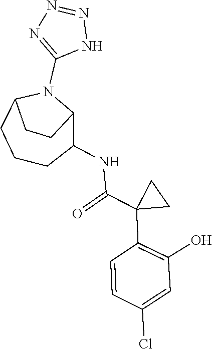 [SMILES: O=C(NC1CCCC2CCC1N2c1nnn[nH]1)C1(c2ccc(Cl)cc2O)CC1] | 403.1 | Compound Rac-92, 92A*, 92B*, 92C* and 92D*, and the corresponding protected —OMe phenol compounds, were prepared according to Protocol C and Example 9 using tert-butyl 2- amino-9-azabicyclo[4.2.1]nonane-9-carboxylate as the protected amine (iv) reagent and 1-(2- hydroxy-4-chlorophenyl)cyclopropane-1- carboxylic acid as the carboxyl (ii) reagent. Rac-92 was then subjected to reverse phase column chromatography (C18 silica gel; mobile phase, Acetonitrile in Water , 0% to 100% gradient in 30 min; detector, UV 254 nm) to provide a mixture of compounds 92A* and 92D* as the first eluting peak, and a mixture of compounds 92B* and 92C* as the second eluting peak. Chiral separation conditions for assumed cis isomers, 92A* and 92D *: CHIRALPAK ID 2*25 cm, 5 um; Mobile Phase A: Hexanes (0.1% trifluoroacetic acid), Mobile Phase B: Ethanol:dichloromethane = 1:1; Flow rate: 20 |
| | N-(9-(1H-tetrazol-5-yl)-9- | | mL/min; Gradient: 20% B to 20% B in 12.5 |
| | azabicyclo[4.2.1]nonan-2-yl)-1-(4-chloro-2- | | min; Wave Length: 220/254 nm. |
| | hydroxyphenyl)cyclopropane-1-carboxamide | | Chiral separation conditions for assumed trans |
| | | | isomers, 92B* and 92C *: CHIRALPAK ID |
| | | | 2*25 cm, 5 um; Mobile Phase A: Hexanes |
| | | | (0.1% trifluoroacetic acid), Mobile Phase B: |
| | | | Ethanol:dichloromethane = 1:1; Flow rate: 20 |
| | | | mL/min; Gradient: 20% B to 20% B in 12.5 |
| | | | min; Wave Length: 220/254 nm. |
| 92A* | N-((1R,2R,6R)-9-(1H-tetrazol-5-yl)-9- | 403.1 | Compound 92A *: Chiral RT(min): 10.55 |
| | azabicyclo[4.2.1]nonan-2-yl)-1-(4-chloro-2- | | |
| | hydroxyphenyl)cyclopropane-1-carboxamide | | |
| 92B* | N-((1R,2S,6R)-9-(1H-tetrazol-5-yl)-9- | 403.1 | Compound 92B*: Chiral RT(min): 7.58 |
| | azabicyclo[4.2.1]nonan-2-yl)-1-(4-chloro-2- | | |
| | hydroxyphenyl)cyclopropane-1-carboxamide | | |
| 92C* | N-((1S,2R,6S)-9-(1H-tetrazol-5-yl)-9- | 403.1 | Compound 92C*: Chiral RT(min): 10.55 |
| | azabicyclo[4.2.1]nonan-2-yl)-1-(4-chloro-2- | | |
| | hydroxyphenyl)cyclopropane-1-carboxamide | | |
| 92D* | N-((1S,2S,6S)-9-(1H-tetrazol-5-yl)-9- | 403.1 | Compound 92D*: Chiral RT(min): 7.58 |
| | azabicyclo[4.2.1]nonan-2-yl)-1-(4-chloro-2- | | |
| | hydroxyphenyl)cyclopropane-1-carboxamide | | |
| Rac-93 | 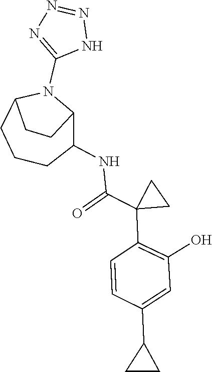 [SMILES: O=C(NC1CCCC2CCC1N2c1nnn[nH]1)C1(c2ccc(C3CC3)cc2O)CC1] | 409.20 | Compound Rac-93, 93A*, 93B*, 93C* and 93D*, and the corresponding protected —OMe phenol compounds, were prepared according to Protocol C and Example 9 using tert-butyl 2- amino-9-azabicyclo[4.2.1]nonane-9-carboxylate as the protected amine (iv) reagent and 1-(2- hydroxy-4-cyclopropylphenyl)cyclopropane-1- carboxylic acid as the carboxyl (ii) reagent. Rac-93 was then subjected to reverse phase column chromatography (XBridge Prep OBD C18 Column, 30*150 mm, 5 μm; Mobile Phase A: Water (10 mmol/L NH₄HCO₃), Mobile Phase B: acetonitrile; Flow rate: 60 mL/min mL/min; Gradient: 18% B to 45% B in 17.8 min; Wave Length: 254 nm/220 nm) to provide a mixture of compounds 93A* and 93D* as the first eluting peak, and a mixture of compounds 93B* and 93C* as the second eluting peak. Chiral separation conditions for assumed cis isomers, 93A* and 93D *: CHIRALPAK ID 2*25 cm, 5 um; Mobile Phase A: Hexanes (0.1% trifluoroacetic acid), Mobile Phase B: isopropanol:dichloromethane = 1:1; Flow rate: |
| | N-(9-(1H-tetrazol-5-yl)-9- | | 20 mL/min; Gradient: 30% B to 30% B in 12 |
| | azabicyclo[4.2.1]nonan-2-yl)-1-(4-cyclopropyl- | | min; Wave Length: 220/254 nm. |
| | 2-hydroxyphenyl)cyclopropane-1-carboxamide | | Chiral separation conditions for assumed trans |
| | | | isomers, 93B* and 93C *: CHIRALPAK ID |
| | | | 2*25 cm, 5 um; Mobile Phase A: Hexanes |
| | | | (0.1% trifluoroacetic acid), Mobile Phase B: |
| | | | isopropanol:dichloromethane = 1:1; Flow rate: |
| | | | 20 mL/min; Gradient: 30% B to 30% B in 12 |
| | | | min; Wave Length: 220/254 nm. |
| 93A* | N-((1R,2R,6R)-9-(1H-tetrazol-5-yl)-9- | 409.20 | Compound 93A *: Chiral RT(min): 5.91 |
| | azabicyclo[4.2.1]nonan-2-yl)-1-(4-cyclopropyl- | | |
| | 2-hydroxyphenyl)cyclopropane-1-carboxamide | | |
| 93B* | N-((1R,2S,6R)-9-(1H-tetrazol-5-yl)-9- | 409.20 | Compound 93B*: Chiral RT(min): 11.06 |
| | azabicyclo[4.2.1]nonan-2-yl)-1-(4-cyclopropyl- | | |
| | 2-hydroxyphenyl)cyclopropane-1-carboxamide | | |
| 93C* | N-((1S,2R,6S)-9-(1H-tetrazol-5-yl)-9- | 409.20 | Compound 93C*: Chiral RT(min): 14.12 |
| | azabicyclo[4.2.1]nonan-2-yl)-1-(4-cyclopropyl- | | |
| | 2-hydroxyphenyl)cyclopropane-1-carboxamide | | |
| 93D* | N-((1S,2S,6S)-9-(1H-tetrazol-5-yl)-9- | 409.20 | Compound 93D*: Chiral RT(min): 9.66 |
| | azabicyclo [4.2.1]nonan-2-yl)-1-(4-cyclopropyl- | | |
| | 2-hydroxyphenyl)cyclopropane-1-carboxamide | | |
| 94A* | 1-(4-(2,2-difluorocyclopropyl)-2- | 419.2 | Compound 94A*, and the corresponding |
| | hydroxyphenyl)-N-((3R,5S)-5-methyl-1-(1H- | | protected —OMe phenol compound, were |
| | tetrazol-5-yl)piperidin-3-yl)cyclopropane-1- | | prepared according to Protocol A and Example |
| | carboxamide | | 33 using (3R,5S)-5-methyl-1-(1H-tetrazol-5- |
| | | | yl)piperidin-3-amine as the amine (i) reagent |
| | | | and 1-(4-(2,2-difluorocyclopropy1)-2- |
| | | | hydroxyphenyl)cyclopropane-1-carboxylic acid |
| | | | as the carboxyl (ii) reagent. |
| 94B* | 1-(4-(2,2-difluorocyclopropyl)-2- | — | Compound 94B*, and the corresponding |
| | hydroxyphenyl)-N-((3S,5S)-5-methyl-1-(1H- | | protected —OMe phenol compound, may be |
| | tetrazol-5-yl)piperidin-3-yl)cyclopropane-1- | | prepared according to Protocol A and Example |
| | carboxamide | | 33 using (3S,5S)-5-methyl-1-(1H-tetrazol-5- |
| | | | yl)piperidin-3-amine as the amine (i) reagent |
| | | | and 1-(4-(2,2-difluorocyclopropyl)-2- |
| | | | hydroxyphenyl)cyclopropane-1-carboxylic acid |
| | | | as the carboxyl (ii) reagent. |
| 94C* | 1-(4-(2,2-difluorocyclopropyl)-2- | — | Compound 94C*, and the corresponding |
| | hydroxyphenyl)-N-((3R,5R)-5-methyl-1-(1H- | | protected —OMe phenol compound, may be |
| | tetrazol-5-yl)piperidin-3-yl)cyclopropane-1- | | prepared according to Protocol A and Example |
| | carboxamide | | 33 using (3R,5R)-5-methyl-1-(1H-tetrazol-5- |
| | | | yl)piperidin-3-amine as the amine (i) reagent |
| | | | and 1-(4-(2,2-difluorocyclopropyl)-2- |
| | | | hydroxyphenyl)cyclopropane-1-carboxylic acid |
| | | | as the carboxyl (ii) reagent. |
| 94D* | 1-(4-(2,2-difluorocyclopropyl)-2- | — | Compound 94D* , and the corresponding |
| | hydroxyphenyl)-N-((3S,5R)-5-methyl-1-(1H- | | protected —OMe phenol compound, may be |
| | tetrazol-5-y1)piperidin-3-yl)cyclopropane-1- | | prepared according to Protocol A and Example |
| | carboxamide | | 33 using (3S,5R)-5-methyl-1-(1H-tetrazol-5- |
| | | | yl)piperidin-3-amine as the amine (i) reagent |
| | | | and 1-(4-(2,2-difluorocyclopropyl)-2- |
| | | | hydroxyphenyl)cyclopropane-1-carboxylic acid |
| | | | as the carboxyl (ii) reagent. |
| 95A* | 1-(4-cyclopropy1-2-hydroxy-6- | 341.1 | Compound 95A*, and the corresponding |
| | (trifluoromethoxy)phenyl)-N-((3R,5S)-5- | | protected —OMe phenol compound, was |
| | methyl-1-(1H-tetrazol-5-yl)piperidin-3- | | prepared according to Protocol A and Example |
| | yl)cyclopropane-1-carboxamide | | 33 using (3R,5S)-5-methyl-1-(1H-tetrazol-5- |
| | | | y1)piperidin-3-amine as the amine (i) reagent |
| | | | and 1-(4-cyclopropyl-2-hydroxy-6- |
| | | | (trifluoromethoxy)phenyl)cyclopropane-1- |
| | | | carboxylic acid as the carboxyl (ii) reagent. |
| 95B* | 1-(4-cyclopropy1-2-hydroxy-6- | — | Compound 95B* , and the corresponding |
| | (trifluoromethoxy)phenyl)-N-((3S,5S)-5-methyl- | | protected —OMe phenol compound, may be |
| | 1-(1H-tetrazol-5-yl)piperidin-3- | | prepared according to Protocol A and Example |
| | yl)cyclopropane-1-carboxamide | | 33 using (3S,5S)-5-methyl-1-(1H-tetrazol-5- |
| | | | yl)piperidin-3-amine as the amine (i) reagent |
| | | | and 1-(4-cyclopropyl-2-hydroxy-6- |
| | | | (trifluoromethoxy)phenyl)cyclopropane-1- |
| | | | carboxylic acid as the carboxyl (ii) reagent. |
| 95C* | 1-(4-cyclopropy1-2-hydroxy-6- | — | Compound 95C*, and the corresponding |
| | (trifluoromethoxy)phenyl)-N-((3R,5R)-5- | | protected —OMe phenol compound, may be |
| | methyl-1-(1H-tetrazol-5-yl)piperidin-3- | | prepared according to Protocol A and Example |
| | yl)cyclopropane-1-carboxamide | | 33 using (3R,5R)-5-methyl-1-(1H-tetrazol-5- |
| | | | yl)piperidin-3-amine as the amine (i) reagent |
| | | | and 1-(4-cyclopropyl-2-hydroxy-6- |
| | | | (trifluoromethoxy)phenyl)cyclopropane-1- |
| | | | carboxylic acid as the carboxyl (ii) reagent. |
| 95D* | 1-(4-cyclopropyl-2-hydroxy-6- | — | Compound 95D* , and the corresponding |
| | (trifluoromethoxy)phenyl)-N-((3S,5R)-5- | | protected —OMe phenol compound, may be |
| | methyl-1-(1H-tetrazol-5-yl)piperidin-3- | | prepared according to Protocol A and Example |
| | yl)cyclopropane-1-carboxamide | | 33 using (3S,5R)-5-methyl-1-(1H-tetrazol-5- |
| | | | yl)piperidin-3-amine as the amine (i) reagent |
| | | | and 1-(4-cyclopropy1-2-hydroxy-6- |
| | | | (trifluoromethoxy)phenyl)cyclopropane-1- |
| | | | carboxylic acid as the carboxyl (ii) reagent. |
| 96A* | 1-(4-cyclopropy1-2-hydroxy-6- | 342.0 | Compound 96A*, and the corresponding |
| | (trifluoromethyl)phenyl)-N-((3R,5S)-5-methyl- | | protected —OMe phenol compound, were |
| | 1-(1H-tetrazol-5-yl)piperidin-3- | | prepared according to Protocol A and Example |
| | yl)cyclopropane-1-carboxamide | | 33 using (3R,5S)-5-methyl-1-(1H-tetrazol-5- |
| | | | yl)piperidin-3-amine as the amine (i) reagent |
| | | | and 1-(4-cyclopropy1-2-hydroxy-6- |
| | | | (trifluoromethyl)phenyl)cyclopropane-1- |
| | | | carboxylic acid as the carboxyl (ii) reagent. |
| 96B* | 1-(4-cyclopropy1-2-hydroxy-6- | — | Compound 96B*, and the corresponding |
| | (trifluoromethyl)phenyl)-N-((3S,5S)-5-methyl- | | protected —OMe phenol compound, may be |
| | 1-(1H-tetrazo1-5-yl)piperidin-3- | | prepared according to Protocol A and Example |
| | yl)cyclopropane-1-carboxamide | | 33 using (3S,5S)-5-methyl-1-(1H-tetrazol-5- |
| | | | yl)piperidin-3-amine as the amine (i) reagent |
| | | | and 1-(4-cyclopropy1-2-hydroxy-6- |
| | | | (trifluoromethyl)phenyl)cyclopropane-1- |
| | | | carboxylic acid as the carboxyl (ii) reagent. |
| 96C* | 1-(4-cyclopropy1-2-hydroxy-6- | — | Compound 96C*, and the corresponding |
| | (trifluoromethyl)phenyl)-N-((3R,5R)-5-methyl- | | protected —OMe phenol compound, may be |
| | 1-(1H-tetrazol-5-yl)piperidin-3- | | prepared according to Protocol A and Example |
| | yl)cyclopropane-1-carboxamide | | 33 using (3R,5R)-5-methyl-1-(1H-tetrazol-5- |
| | | | yl)piperidin-3-amine as the amine (i) reagent |
| | | | and 1-(4-cyclopropy1-2-hydroxy-6- |
| | | | (trifluoromethyl)phenyl)cyclopropane-1- |
| | | | carboxylic acid as the carboxyl (ii) reagent. |
| 96D* | 1-(4-cyclopropy1-2-hydroxy-6- | — | Compound 96C*, and the corresponding |
| | (trifluoromethyl)phenyl)-N-((3S,5R)-5-methyl- | | protected —OMe phenol compound, may be |
| | 1-(1H-tetrazo1-5-yl)piperidin-3- | | prepared according to Protocol A and Example |
| | yl)cyclopropane-1-carboxamide | | 33 using (3S,5R)-5-methyl-1-(1H-tetrazol-5- |
| | | | yl)piperidin-3-amine as the amine (i) reagent |
| | | | and 1-(4-cyclopropyl-2-hydroxy-6- |
| | | | (trifluoromethyl)phenyl) cyclopropane-1- |
| | | | carboxylic acid as the carboxyl (ii) reagent. |
| 97A* | 1-(4-cyclopropyl-2-(difluoromethyl)-6- | 433.2 | Compound 97A*, and the corresponding |
| | hydroxyphenyl)-N-((3R,5S)-5-methyl-1-(1H- | | protected —OMe phenol compound, were |
| | tetrazol-5-yl)piperidin-3-yl)cyclopropane-1- | | prepared according to Protocol A and Example |
| | carboxamide | | 33 using (3R,5S)-5-methyl-1-(1H-tetrazol-5- |
| | | | yl)piperidin-3-amine as the amine (i) reagent |
| | | | and 1-(4-cyclopropyl-2-(difluoromethyl)-6- |
| | | | hydroxyphenyl) cyclopropane-1-carboxylic acid |
| | | | as the carboxyl (ii) reagent. |
| 97B* | 1-(4-cyclopropyl-2-(difluoromethyl)-6- | — | Compound 97B*, and the corresponding |
| | hydroxyphenyl)-N-((3S,5S)-5-methyl-1-(1H- | | protected —OMe phenol compound, may be |
| | tetrazol-5-yl)piperidin-3-yl)cyclopropane-1- | | prepared according to Protocol A and Example |
| | carboxamide | | 33 using (3S,5S)-5-methyl-1-(1H-tetrazol-5- |
| | | | yl)piperidin-3-amine as the amine (i) reagent |
| | | | and 1-(4-cyclopropyl-2-(difluoromethy1)-6- |
| | | | hydroxyphenyl) cyclopropane-1-carboxylic acid |
| | | | as the carboxyl (ii) reagent. |
| 97C* | 1-(4-cyclopropy1-2-(difluoromethyl)-6- | — | Compound 97C*, and the corresponding |
| | hydroxyphenyl)-N-((3R,5R)-5-methyl-1-(1H- | | protected —OMe phenol compound, may be |
| | tetrazol-5-yl)piperidin-3-yl)cyclopropane-1- | | prepared according to Protocol A and Example |
| | carboxamide | | 33 using (3R,5R)-5-methyl-1-(1H-tetrazol-5- |
| | | | yl)piperidin-3-amine as the amine (i) reagent |
| | | | and 1-(4-cyclopropyl-2-(difluoromethyl)-6- |
| | | | hydroxyphenyl) cyclopropane-1-carboxylic acid |
| | | | as the carboxyl (ii) reagent. |
| 97D* | 1-(4-cyclopropy1-2-(difluoromethyl)-6- | — | Compound 97D* , and the corresponding |
| | hydroxyphenyl)-N-((3S,5R)-5-methyl-1-(1H- | | protected —OMe phenol compound, may be |
| | tetrazol-5-yl)piperidin-3-yl)cyclopropane-1- | | prepared according to Protocol A and Example |
| | carboxamide | | 33 using (3S,5R)-5-methyl-1-(1H-tetrazol-5- |
| | | | yl)piperidin-3-amine as the amine (i) reagent |
| | | | and 1-(4-cyclopropy1-2-(difluoromethy1)-6- |
| | | | hydroxyphenyl)cyclopropane-1-carboxylic acid |
| | | | as the carboxyl (ii) reagent. |
| Rac-98 | 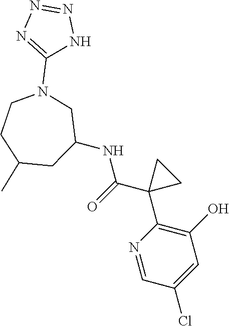 [SMILES: CC1CCN(c2nnn[nH]2)CC(NC(=O)C2(c3ncc(Cl)cc3O)CC2)C1] | 392.1 | Compound Rac-98, 98A*, 98*, 98C* and 98D*, and the corresponding protected —OMe phenol compounds, were prepared according to Protocol A and Example 35 using 6-methyl-1- (1H-tetrazol-5-yl)azepan-3-amine as the amine (i) reagent and 1-(5-chloro-3-hydroxypyridin-2- yl)cyclopropane-1-carboxylic acid as the carboxyl (ii) reagent. Rac-98 was then subjected to prep chiral HPLC using the following conditions: Xselect CSH F- Phenyl OBD column, 19*250 mm, 5 μm; Mobile Phase A: Water(0.05% trifluoroacetic acid), Mobile Phase B: acetonitrile; Flow rate: 20 mL/min; Gradient: 15% B to 20% B in 10 min; |
| | 1-(5-chloro-3-hydroxypyridin-2-yl)-N-(5- | | Wave Length: 254 nm to provide a mixture of |
| | methyl-1-(1H-tetrazol-5-yl)azepan-3- | | compounds 98B* and 98C* as the first eluting |
| | yl)cyclopropane-1-carboxamide | | peak, and a mixture of compounds 98A* and |
| | | | 98D* as the second eluting peak. |
| 98A* | 1-(5-chloro-3-hydroxypyridin-2-yl)-N-((3R,5R)- | 392.1 | Compound 98A* = second eluting peak |
| | 5-methyl-1-(1H-tetrazol-5-yl)azepan-3- | | |
| | yl)cyclopropane-1-carboxamide | | |
| 98B* | 1-(5-chloro-3-hydroxypyridin-2-yl)-N-((3S,5R)- | 392.1 | Compound 98B* = first eluting peak |
| | 5-methyl-1-(1H-tetrazol-5-yl)azepan-3- | | |
| | yl)cyclopropane-1-carboxamide | | |
| 98C* | 1-(5-chloro-3-hydroxypyridin-2-yl)-N-((3R,5S)- | 392.1 | Compound 98C* = first eluting peak |
| | 5-methyl-1-(1H-tetrazol-5-yl)azepan-3- | | |
| | yl)cyclopropane-1-carboxamide | | |
| 98D* | 1-(5-chloro-3-hydroxypyridin-2-yl)-N-((3S,5S)- | 392.1 | Compound 98D* = second eluting peak |
| | 5-methyl-1-(1H-tetrazol-5-yl)azepan-3- | | |
| | yl)cyclopropane-1-carboxamide | | |
| Rac-99 | 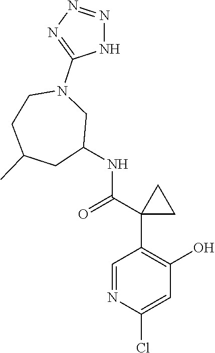 [SMILES: CC1CCN(c2nnn[nH]2)CC(NC(=O)C2(c3cnc(Cl)cc3O)CC2)C1] | 392.1 | Rac-99, 99A*, 99B*, 99C* and 99D*, and the corresponding protected —OMe phenol compounds, were prepared according to Protocol A and Example 35 using 6-methyl-1- (1H-tetrazol-5-yl)azepan-3-amine as the amine (i) reagent and 1-(6-chloro-4-hydroxypyridin-3- yl)cyclopropane-1-carboxylic acid as the carboxyl (ii) reagent. Rac-99 was then subjected to reverse phase column chromatography (C18 silica gel; mobile phase, Acetonitrile in Water , 0% to 100% gradient in 30 min; detector, UV 254 nm) to provide a mixture of compounds 99B* and 99C* as the first eluting peak, and a mixture of compounds 99A* and 99D* as the second eluting peak. Chiral separation conditions for assumed trans isomers, 99B* and 99C *: CHIRAL ART Cellulose-SC, 2*25 cm, 5 μm; Mobile Phase A: Hexanes (0.1% trifluoroacetic acid), Mobile Phase B: isopropanol:dichloromethane = 1:1; Flow rate: 20 mL/min; Gradient: 50% B to 50% |
| | 1-(6-chloro-4-hydroxypyridin-3-yl)-N-(5- | | B in 10 min; Wave Length: 220/254 nm. |
| | methyl-1-(1H-tetrazol-5-yl)azepan-3- | | Chiral separation conditions for assumed cis |
| | yl)cyclopropane-1-carboxamide | | isomers, 99A* and 99D *: CHIRAL ART |
| | | | Cellulose-SC, 2*25 cm, 5 μm; Mobile Phase A: |
| | | | Hexanes (0.1% trifluoroacetic acid), Mobile |
| | | | Phase B: isopropanol:dichloromethane =1: 1; |
| | | | Flow rate: 20 mL/min; Gradient: 50% B to 50% |
| | | | B in 11 min; Wave Length: 220/254 nm. |
| 99A* | 1-(6-chloro-4-hydroxypyridin-3-yl)-N-((3R,5R)- | 392.1 | Compound 99A*: Chiral RT(min): 7.98 |
| | 5-methyl-1-(1H-tetrazol-5-yl)azepan-3- | | |
| | yl)cyclopropane-1-carboxamide | | |
| 99B* | 1-(6-chloro-4-hydroxypyridin-3-yl)-N-((3S,5R)- | 392.1 | Compound 99B*: Chiral RT(min): 6.21 |
| | 5-methy1-1-(1H-tetrazol-5-yl)azepan-3- | | |
| | yl)cyclopropane-1-carboxamide | | |
| 99C* | 1-(6-chloro-4-hydroxypyridin-3-yl)-N-((3R,5S)- | 392.1 | Compound 99C*: Chiral RT(min): 8.46 |
| | 5-methyl-1-(1H-tetrazol-5-yl)azepan-3- | | |
| | yl)cyclopropane-1-carboxamide | | |
| 99D* | 1-(6-chloro-4-hydroxypyridin-3-yl)-N-((3S,5S)- | 392.1 | Compound 99D*: Chiral RT(min): 9.95 |
| | 5-methyl-1-(1H-tetrazol-5-yl)azepan-3- | | |
| | yl)cyclopropane-1-carboxamide | | |
| Rac- 100 | 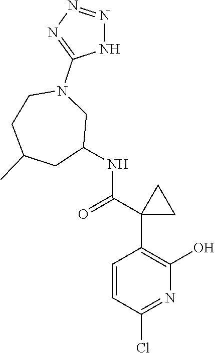 [SMILES: CC1CCN(c2nnn[nH]2)CC(NC(=O)C2(c3ccc(Cl)nc3O)CC2)C1] | 392.1 | Rac-100, 100A*, 100B*, 100C* and 100D*, and the corresponding protected —OMe phenol compounds, were prepared according to Protocol A and Example 35 using 6-methyl-1- (1H-tetrazol-5-yl)azepan-3-amine as the amine (i) reagent and 1-(6-chloro-2-hydroxypyridin-3- yl)cyclopropane-1-carboxylic acid as the carboxyl (ii) reagent. Rac-100 was then subjected to reverse phase column chromatography (XBridge Prep Phenyl OBD Column, 19*250 mm, 5 μm; mobile phase, Water(10 mmol/L NH₄HCO₃) and methanol (45% up to 60% in 10 min); Wave Length: 220/254 nm) to provide a mixture of compounds 100A* and 100D* as the first eluting peak, and a mixture of compounds 101B* and 101C* as the second eluting peak. Chiral separation conditions for assumed cis isomers, 100A* and 100D *: CHIRALPAK IG, 2*25 cm, 5 μm; Mobile Phase A: Hexanes (0.1% trifluoroacetic acid), Mobile Phase B: Ethanol:dichloromethane = 1:1; Flow rate: 20 |
| | 1-(6-chloro-2-hydroxypyridin-3-yl)-N-(5- | | mL/min; Gradient: 20% B to 20% B in 25 min; |
| | methyl-1-(1H-tetrazol-5-yl)azepan-3- | | Wave Length: 220/254 nm. |
| | yl)cyclopropane-1-carboxamide | | Chiral separation conditions for assumed trans |
| | | | isomers, 100B* and 100C *: CHIRALPAK IE, |
| | | | 2*25 cm, 5 um; Mobile Phase A: Hexanes |
| | | | (0.1% trifluoroacetic acid), Mobile Phase B: |
| | | | isopropanol:dichloromethane = 1:1; Flow rate: |
| | | | 20 mL/min; Gradient: 30% B to 30% B in 25 |
| | | | min; Wave Length: 220/254 nm. |
| 100A* | 1-(6-chloro-2-hydroxypyridin-3-yl)-N-((3R,5R)- | 392.1 | Compound 100A *: Chiral RT(min): 18.08 |
| | 5-methyl-1-(1H-tetrazol-5-yl)azepan-3- | | |
| | yl)cyclopropane-1-carboxamide | | |
| 100B* | 1-(6-chloro-2-hydroxypyridin-3-yl)-N-((3S,5R)- | 392.1 | Compound 100B *: Chiral RT(min): 18.48 |
| | 5-methy1-1-(1H-tetrazol-5-yl)azepan-3- | | |
| | yl)cyclopropane-1-carboxamide | | |
| 100C* | 1-(6-chloro-2-hydroxypyridin-3-yl)-N-((3R,5S)- | 392.1 | Compound 100C *: Chiral RT(min): 22.24 |
| | 5-methyl-1-(1H-tetrazol-5-yl)azepan-3- | | |
| | yl)cyclopropane-1-carboxamide | | |
| 100D* | 1-(6-chloro-2-hydroxypyridin-3-yl)-N-((3S,5S)- | 392.1 | Compound 100D *: Chiral RT(min): 21.72 |
| | 5-methyl-1-(1H-tetrazol-5-yl)azepan-3- | | |
| | yl)cyclopropane-1-carboxamide | | |
| Rac- 102 | 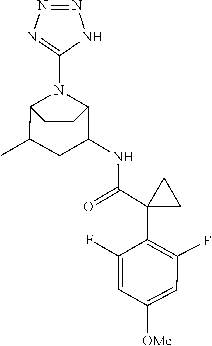 [SMILES: COc1cc(F)c(C2(C(=O)NC3CC(C)C4CCC3N4c3nnn[nH]3)CC2)c(F)c1] | | Rac-102 and Compounds 102B* and 102C* may prepared according to Protocol A and Example 38 using 4-methyl-8-(1H-tetrazol-5- yl)-8-azabicyclo[3.2.1]octan-2-amine as the amine (i) reagent and 1-(2,6-difluoro-4- methoxyphenyl)cyclopropane-1-carboxylic acid as the carboxyl (ii) reagent. |
| | 1-(2,6-difluoro-4-methoxyphenyl)-N-(4-methyl- | | |
| | 8-(1H-tetrazol-5-yl)-8-azabicyclo[3.2.1]octan-2- | | |
| | yl)cyclopropane-1-carboxamide | | |
| 102B* | 1-(2,6-difluoro-4-methoxyphenyl)-N- | — | |
| | ((1R,2S,4R,5S)-4-methy1-8-(1H-tetrazol-5-yl)- | | |
| | 8-azabicyclo[3.2.1]octan-2-yl)cyclopropane-1- | | |
| | carboxamide | | |
| 102C* | 1-(2,6-difluoro-4-methoxyphenyl)-N- | — | |
| | ((1S,2R,4S,5R)-4-methyl-8-(1H-tetrazol-5-yl)- | | |
| | 8-azabicyclo[3.2.1]octan-2-yl)cyclopropane-1- | | |
| | carboxamide | | |
| 105 | 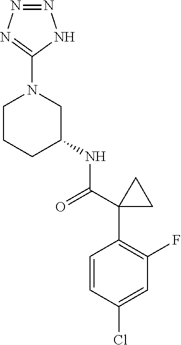 [SMILES: O=C(N[C@@H]1CCCN(c2nnn[nH]2)C1)C1(c2ccc(Cl)cc2F)CC1] | 365.2 | Compound 105 was prepared according to Protocol A and Example 15 using (3R)-1-(1H- 1,2,3,4-tetrazol-5-yl)piperidin-3-amine as the amine (i) reagent and 1-(4-chloro-2- fluorophenyl)cyclopropane-1-carboxylic acid as the carboxyl (ii) reagent. |
| | (R)-N-(1-(1H-tetrazol-5-yl)piperidin-3-yl)-1-(4- | | |
| | chloro-2-fluorophenyl)cyclopropane-1- | | |
| | carboxamide | | |
| 106 | 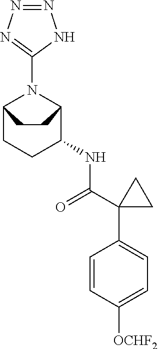 [SMILES: O=C(N[C@@H]1CC[C@H]2CC[C@@H]1N2c1nnn[nH]1)C1(c2ccc(OC(F)F)cc2)CC1] | 405.2 | Compound 106 was prepared according to Protocol A and Example 34 using (1S,2R,5S)-8- (2H-tetrazol-5-yl)-8-azabicyclo[3.2.1]octan-2- amine as the amine (i) reagent and 1-(4- (difluoromethoxy)phenyl)cyclopropane-1- carboxylic acid as the carboxyl (ii) reagent. |
| | N-((1S,2R,5S)-8-(1H-tetrazol-5-yl)-8- | | |
| | azabicyclo[3.2.1]octan-2-yl)-1-(4- | | |
| | (difluoromethoxy)phenyl)cyclopropane-1- | | |
| | carboxamide | | |

### Biological Assay Methods

### Scintillation Proximity (SPA) NLRP3 Assay

The objective of this assay to is demonstrate binding of a test compound to human NLRP3 protein by displacement of 3H-MCC950, a selective inhibitor of NLRP3. Improved NLRP3 binding correlates with increased affinity for NLRP3 protein and, by extension, increased ability to interfere with its function.

SPA experiments were conducted in a total volume of 20 μL assay buffer containing 20 mM HEPES pH 7.5, 150 mM NaCl, 5 mM MgCl₂, 0.01% (v/v) Tween-20, 0.01% (w/v) bovine serum albumin (BSA) and 100 μM of adenosine diphosphate (ADP). A human NLRP3 protein (350 nM) (see, e.g., Sharif et al., Nature (2019) 570:338-343; Hochheiser et al., Nature (2022) 604:184-189; Adreeva et al., Cell (2021) 184:6299-6312; Wang et al., J. Exp. Med. (2021) 219:e20211147) was incubated with various concentration of compounds in 5% dimethyl sulfoxide (DMSO) final for 15 minutes at room temperature in a white half-area 96 well OptiPlate (PerkinElmer #6002290) before adding 125 nM final of 3H-MCC950. After the addition of radiolabeled MCC950, the plate was incubated for 60 minutes at room temperature before adding 0.25 mg/ml of copper-coated polyvinyltoluene SPA beads (PerkinElmer #RPNQ0095). The plate was then transferred to a microplate counter (PerkinElmer 2450 Microbeta) to collect binding data. Binding data was analyzed 6 hrs post-PVT bead addition to allow sufficient time to reach equilibrium. Specific binding was calculated from averaged duplicates by subtracting signal from wells without NLRP3 protein.

SPA NLRP3 IC₅₀ assay results are provided in Table B. “A” activity=<1 uM; “B” activity=1-10 uM; “C” activity=>10-50 uM; “D” activity=>50 uM-100 uM; and “E” activity=>100 uM.

### Human PBMC NLRP3 Assay

The objective of this assay to is demonstrate if a test compound is able to interfere with human NLRP3 function in a cellular system.

Reagents: Human PBMCs (Normal): iXCells Cat #10HU-003; RPMI 1640 medium with GlutaMAX: ThermoFisher Cat #61870127 (Complete Media: 4.5 g/L D-glucose, 10% FBS, 100 mM NaPyr, 1% Pen/Strep, 10 mM HEPES and 0.05 mM of ®-mercaptoethanol; Assay Media: 4.5 g/L D-glucose, 100 mM NaPyr, 10% Pen/Strep, 10 mM HEPES and 0.05 mM of mercaptoethanol; 96-well V-bottom Plates: Costar Cat #3894; LPS (E. coli 026:B6): Sigma Cat #L2654, stock 5 mg/mL in PBS; ATP: Sigma Cat #A6419, prepared in a 250 mM stock in 1 M HEPES (adjusted to pH 7.4).

Cryopreserved PBMCs were rapidly thawed in a 37° C. water bath for 2 min. Cells were then centrifuged at 1200 RPM for 5 min and resuspended in −50 mL of fresh RPMI 1640 Complete Medium. A count was undertaken using a hemocytometer and adjusted to 2.5×10′ cells/mL. V-shaped 96-well plates were seeded with 200 μL of PBMCs (5×10⁴) per well and subsequently incubated overnight at 37° C. with 5% CO₂. Assay Media was then prepared containing 100 ng/ml of LPS. PBMCs were then centrifuged at 1,200 RPM for 5 min, serum containing media was aspirated, and 150 μL/well of Assay Media+LPS was immediately added. Assay Media without LPS was added in the untreated control wells. Cells were then primed with LPS for 4 h at 37° C. with 5% CO₂. A concentration response curve (CRC) was prepared of 1000× test compound in 100% DMSO. The CRC was then diluted 1:50 in Assay Media and then further diluted by 1:5× in Assay Media resulting in a final 4×CRC in 0.4% DMSO/Assay Media. 50 μL/well of 4× test compound CRC or vehicle (0.4% DMSO/Assay Media) was then transferred into each well and subsequently incubated for 30 min at 37° C. with 5% CO₂. Cells were stimulated by adding 4 μL of 250 mM ATP, using a 250 mM stock prepared in 1 M Hepes without further dilution (for a final concentration of 5 mM), for 1 h at 37° C. with 5% CO₂ Plates were then centrifuged at 1200 RPM for 5 min; and 50 μl of the media was transferred to a clean 96-well storage plate for cytokine measurements using the mesoscale platform, and stored at −80° C. until analyzed. Compounds were added 30 minutes before priming the cells with LPS when TNFα was required to be quantified in the cytokine panel to confirm selectivity.

PBMC NLRP3 IC₅₀ assay results, as a measurement of cellular potency, are provided in Table B. “A” activity=<0.01 uM; “B” activity=0.01-0.1 uM; “C” activity=>0.1-0.5 uM; “D” activity=>0.5 uM.

Human Whole Blood (hWB) NLRP3 Assay

The objective of this assay to is demonstrate if a test compound is able to interfere with human NLRP3 function in a whole blood system.

Human whole blood is drawn from healthy volunteers after obtaining written informed consent.

Heparin lithium coated tubes are used to collect blood from volunteers. Blood samples are distributed on 96 well plates using 90 μl per well.

Priming procedure: Priming is performed by adding 5 μl of LPS (026:B6; Sigma L-2654) at a final concentration of 1 μg/ml for 4.5 hours in a humidified incubator with 37° C., 5% CO₂. Thirty minutes prior to NLRP3 activation, 5 μl of a 20× compound solution or vehicle (2% dimethylsulfoxide (DMSO)) is added to each well and plates were incubated on a shaker (450 rpm) in a humidified incubator with 37° C., 5% CO₂.

Alternative priming procedure: Thirty minutes prior to LPS priming, 5 μl of a 20× compound solution or vehicle (2% dimethylsulfoxide (DMSO)) is added to each well and plates were incubated on a shaker (450 rpm) in a humidified incubator with 37° C., 5% CO₂. Priming is performed by adding 5 μl of LPS (026:B6; Sigma L-2654) at a final concentration of 1 μg/ml for 5 hours in a humidified incubator with 37° C., 5% CO₂.

Activation is then performed by adding 3.3 μl of a 31×ATP solution per well. At the end of the 30 minutes stimulation, the plates are centrifuged (800 g, 10 min, room temperature) and the plasma from each well is frozen at −80° C. IL-1β levels in the supernatant were analyzed using a mesoscale discovery assay (MSD K151TUK) according to the manufacturers' instructions.

There is no detectable difference in assay results using the priming procedure versus the alternative priming procedure.

hWB NLRP3 IC₅₀ assay results, as a measurement of whole blood potency, are provided in Table B. “A” activity=<1 uM; “B” activity=1-2 uM; “C” activity=>2-3 uM; “D” activity=>3 uM.

Mouse Whole Blood (mWB) NLRP3 Assay

The objective of this assay to is demonstrate if a test compound is able to interfere with mouse NLRP3 function in a whole blood system.

Reagents: The following reagents were used: blood collection tubes (Heparin); U-bottom 96-well tissue culture (Falcon 353077); HBSS for LPS, ATP and compound dilutions (Gibco 24020-117); and LPS, E. coli serotype 026:B6 (Sigma L-2654).

Mouse IL-1b MSD assay: Blood was drawn from female CD1 mice (9 to 10 weeks) by cardiac puncture. Blood was plated (135 μL) per well in 96-well U-bottom plates. 7.5 μL of 20×LPS (20 μg/mL, final concentration of 1 μg/mL) was added and mixed by gentle pipetting, and the mixture was incubated in a TC incubator for five hours. 7.5 μL of 20× compound or vehicle per well was added and mixed by gentle pipetting. Compounds were diluted 1/50 in HBSS to prepare a 20× dilution curve in 2 DMSO.

The mixture was incubated for 30 minutes in a TC incubator with shaking (450 rpm). 5 μl of 31×ATP (155 mM, final concentration of 5 mM) was added and mixed by gentle pipetting. The mixture was incubated in a TC incubator for 30 minutes with shaking (450 rpm). The plate was centrifuged for 10 minutes at 800×g and −70 μL of plasma was removed. Plasma was frozen if required. Mouse IL-1b was analyzed with IL-1b MSD assay (K152TUK).

Compounds were added 30 minutes before priming the cells with LPS when TNFα was required to be quantified in the cytokine panel to confirm selectivity.

mWB NLRP3 IC₅₀ assay results, as a measurement of whole blood potency, are provided in Table B. “A” activity=<1 uM; “B” activity=1-2 uM; “C” activity=>2-3 uM; “D” activity=>3 uM.

### Biological Assay Data

Various compounds described herein were tested according to the above-described assays. Dashed (--) line means no data available.

TABLE B
Activity Data

| Cmp. # | SPA IC₅₀ (uM) | PBMC IC₅₀ (uM) | mWB IC₅₀ (uM) | hWB IC50 (uM) |
|---|---|---|---|---|
| Rac-1 | A | C | — | — |
| 2A* | A | B | — | — |
| 2B* | B | — | — | — |
| 2C* | C | — | — | — |
| 2D* | B | — | — | — |
| 3A* | A | B | — | — |
| 3B* | B | — | — | — |
| 3C* | E | — | — | — |
| 3D* | B | — | — | — |
| 4A* | A | B | — | — |
| 4B* | C | — | — | — |
| 4C* | D | — | — | — |
| 4D* | B | — | — | — |
| 5A* | A | B | — | — |
| 5B* | C | — | — | — |
| 5C* | E | — | — | — |
| 5D* | C | — | — | — |
| 6A* | B | — | — | — |
| 6B* | D | — | — | — |
| 6C* | E | — | — | — |
| 6D* | D | — | — | — |
| 7A* | A | B | — | — |
| 7B* | C | — | — | — |
| 7C* | D | — | — | — |
| 7D* | B | — | — | — |
| 8A* | B | — | — | — |
| 8B* | C | — | — | — |
| 8C* | C | — | — | — |
| 8D* | C | — | — | — |
| 9A* | B | — | — | — |
| 9B* | E | — | — | — |
| 9C* | E | — | — | — |
| 9D* | C | — | — | — |
| 10A | B | D | B | D |
| 10B | E | — | — | — |
| 11A* | E | — | — | — |
| 11B* | E | — | — | — |
| 12A* | A | A | A | A |
| 12B* | C | — | — | — |
| 12C* | E | — | — | — |
| 12D* | C | — | — | — |
| Rac-13A*/D* | B | — | — | — |
| Rac-13B*/C* | D | — | — | — |
| 14A* | A | B | — | B |
| 14B* | C | — | — | — |
| 15A | B | C | — | A |
| 15B | — | — | — | — |
| 16A | A | A | A | A |
| 16B | — | — | — | — |
| 16C | — | — | — | — |
| 16D | — | — | — | — |
| 17A | A | B | — | A |
| 17B | — | — | — | — |
| 17C | — | — | — | — |
| 17D | — | — | — | — |
| 18A* | A | A | — | A |
| 18B* | B | — | — | — |
| 18C* | C | C | — | — |
| 18D* | A | C | — | — |
| 19A | A | B | — | A |
| 19B | — | — | — | — |
| 19C | — | — | — | — |
| 19D | — | — | — | — |
| 20A | A | B | — | A |
| 20B | — | — | — | — |
| 20C | — | — | — | — |
| 20D | — | — | — | — |
| 21A* | A | A | — | A |
| 21B* | B | — | — | — |
| 21C* | E | — | — | — |
| 21D* | C | — | — | — |
| 22A* | A | A | — | A |
| 22B* | B | — | — | — |
| 22C* | C | — | — | — |
| 22D* | B | — | — | — |
| 23A | A | B | — | A |
| 23B | — | — | — | — |
| 23C | — | — | — | — |
| 23D | — | — | — | — |
| 24A | B | — | — | — |
| 24B | — | — | — | — |
| 24C | — | — | — | — |
| 24D | — | — | — | — |
| 25A | A | C | — | D |
| 25B | — | — | — | — |
| 25C | — | — | — | — |
| 25D | — | — | — | — |
| 26A | B | — | — | — |
| 26B | — | — | — | — |
| 26C | — | — | — | — |
| 26D | — | — | — | — |
| 27A* | A | A | — | A |
| 27B* | B | — | — | — |
| 27C* | D | — | — | — |
| 27D* | B | — | — | — |
| 28A* | A | B | — | D |
| 28B* | B | — | — | — |
| 28C* | E | — | — | — |
| 28D* | B | — | — | — |
| 29A* | A | B | — | B |
| 29B* | — | — | — | — |
| 29C* | — | — | — | — |
| 29D* | C | — | — | — |
| 30A* | A | A | — | — |
| 30B* | A | B | — | — |
| 30C* | E | — | — | — |
| 30D* | B | — | — | — |
| 31A* | A | A | — | C |
| 31B* | A | B | — | — |
| 31C* | C | — | — | — |
| 31D* | B | — | — | — |
| 32A* | A | A | — | — |
| 32B* | B | — | — | — |
| 32C* | D | — | — | — |
| 32D* | C | — | — | — |
| 33A | A | B | — | D |
| 33B | — | — | — | — |
| 33C | — | — | — | — |
| 33D | — | — | — | — |
| 34A* | — | — | — | — |
| 34B* | B | — | — | — |
| 34C* | A | B | A | A |
| 34D* | — | — | — | — |
| 35A* | A | A | A | A |
| 35B* | D | — | — | — |
| 35C* | E | — | — | — |
| 35D* | C | — | — | — |
| 35E* | B | — | — | D |
| 35F* | D | — | — | — |
| 35G* | E | — | — | — |
| 35H* | E | — | — | — |
| 36A* | A | — | — | — |
| 36B* | B | — | — | — |
| 36C* | E | — | — | — |
| 36D* | E | — | — | — |
| 37A* | A | — | — | B |
| 37B* | C | — | — | — |
| 37C* | E | — | — | — |
| 37D* | E | — | — | — |
| 38A | — | — | — | — |
| 38B | — | — | — | — |
| 39A | B | — | B | B |
| 39B | — | — | — | — |
| 40A | E | — | — | — |
| 40B | — | — | — | — |
| 40C | E | — | — | — |
| 40D | — | — | — | — |
| 41A | — | — | — | — |
| 41B | — | — | — | — |
| 41C | E | — | — | — |
| 41D | — | — | — | — |
| 42A* | A | A | — | — |
| 42B* | C | — | — | — |
| 43A* | A | — | — | A |
| 43B* | A | — | — | D |
| 43C* | E | — | — | — |
| 43D* | C | — | — | — |
| 44A | E | — | — | — |
| 44B | — | — | — | — |
| 44C | C | — | — | — |
| 44D | — | — | — | — |
| 45A | B | — | A | C |
| 45B | — | — | — | — |
| 45C | — | — | — | — |
| 45D | — | — | — | — |
| 46A | A | B | A | C |
| 46B | — | — | — | — |
| 46C | C | — | — | — |
| 46D | — | — | — | — |
| 47A | A | A | A | A |
| 47B | — | — | — | — |
| 47C | — | — | — | — |
| 47D | — | — | — | — |
| 48A | A | — | — | A |
| 48B | — | — | — | — |
| 48C | — | — | — | — |
| 48D | — | — | — | — |
| 49A | E | — | — | — |
| 49B | — | — | — | — |
| 49C | B | C | C | C |
| 49D | — | — | — | — |
| 50A | C | — | — | — |
| 50B | — | — | — | — |
| 50C | C | — | — | — |
| 50D | — | — | — | — |
| 51A | B | B | B | A |
| 51B | — | — | — | — |
| 52A | A | — | — | A |
| 52B | — | — | — | — |
| 53A* | A | — | A | A |
| 53B* | D | — | — | — |
| 54A* | A | B | — | — |
| 54B* | C | — | — | — |
| 54C* | E | — | — | — |
| 54D* | C | — | — | — |
| 55A* | A | B | — | — |
| 55B* | C | — | — | — |
| 55C* | D | — | — | — |
| 55D* | D | — | — | — |
| 56A* | A | A | — | B |
| 56B* | B | — | — | — |
| 56C* | D | — | — | — |
| 56D* | C | — | — | — |
| 57A* | A | B | — | B |
| 57B* | B | — | — | — |
| 57C* | E | — | — | — |
| 57D* | C | — | — | — |
| 58A* | A | B | — | A |
| 58B* | B | — | — | — |
| 58C* | E | — | — | — |
| 58D* | B | — | — | — |
| 59A* | A | A | — | A |
| 59B* | B | — | — | — |
| 59C* | D | — | — | — |
| 59D* | B | — | — | — |
| 60A* | A | A | — | A |
| 60B* | A | B | — | A |
| 60C* | B | — | — | — |
| 60D* | A | — | — | — |
| 61A* | A | B | — | A |
| 61B* | B | — | — | — |
| 61C* | D | — | — | — |
| 61D* | C | — | — | — |
| 62A* | A | A | — | A |
| 62B* | A | B | — | B |
| 62C* | B | — | — | — |
| 62D* | B | — | — | — |
| 63A* | A | A | — | A |
| 63B* | A | B | — | A |
| 63C* | C | — | — | — |
| 63D* | A | — | — | — |
| 64A* | A | B | — | A |
| 64B* | C | — | — | — |
| 64C* | D | — | — | — |
| 64D* | B | — | — | — |
| 65A* | A | B | — | — |
| 65B* | C | — | — | — |
| 65C* | C | — | — | — |
| 65D* | C | — | — | — |
| 66A* | A | A | — | A |
| 66B* | B | — | — | — |
| 66C* | E | — | — | — |
| 66D* | C | — | — | — |
| 67A* | A | A | — | A |
| 67B* | A | — | — | — |
| 67C* | C | — | — | — |
| 67D* | B | — | — | — |
| 68A* | A | A | — | — |
| 68B* | B | — | — | — |
| 68C* | E | — | — | — |
| 68D* | B | — | — | — |
| 69A* | A | B | — | — |
| 69B* | C | — | — | — |
| 69C* | E | — | — | — |
| 69D* | C | — | — | — |
| 70A* | A | A | — | — |
| 70B* | A | B | — | — |
| 70C* | C | — | — | — |
| 70D* | A | B | — | — |
| 71A* | A | B | — | — |
| 71B* | D | — | — | — |
| 71C* | D | — | — | — |
| 71D* | C | — | — | — |
| 72A* | A | A | — | D |
| 72B* | B | — | — | — |
| 72C* | E | — | — | — |
| 72D* | C | — | — | — |
| 73A* | A | B | — | — |
| 73B* | B | — | — | — |
| 73C* | C | — | — | — |
| 73D* | B | — | — | — |
| 74A* | A | A | — | A |
| 74B* | B | — | — | — |
| 74C* | C | — | — | — |
| 74D* | B | — | — | — |
| 75A* | — | — | — | — |
| 75B* | B | — | — | — |
| 75C* | A | B | — | B |
| 75D* | — | — | — | — |
| 76A* | — | — | — | — |
| 76B* | C | — | — | — |
| 76C* | A | C | — | B |
| 76D* | — | — | — | — |
| 77A* | — | — | — | — |
| 77B* | C | — | — | — |
| 77C* | A | B | — | A |
| 77D* | — | — | — | — |
| 78A* | — | — | — | — |
| 78B* | B | — | — | — |
| 78C* | A | B | — | C |
| 78D* | — | — | — | — |
| 79A* | A | A | A | A |
| 79B* | C | — | — | — |
| 79C* | C | — | — | — |
| 79D* | C | — | — | — |
| 80A* | A | B | — | A |
| 80B* | C | — | — | — |
| 80C* | E | — | — | — |
| 80D* | D | — | — | — |
| 81A* | A | B | — | A |
| 81B* | D | — | — | — |
| 81C* | D | — | — | — |
| 81D* | C | — | — | — |
| 82A* | A | A | — | A |
| 82B* | E | — | — | — |
| 82C* | E | — | — | — |
| 82D* | E | — | — | — |
| 83A* | A | A | — | A |
| 83B* | C | — | — | — |
| 83C* | E | — | — | — |
| 83D* | E | — | — | — |
| 84A* | A | A | — | A |
| 84B* | C | — | — | — |
| 84C* | E | — | — | — |
| 84D* | E | — | — | — |
| 85A* | A | A | — | A |
| 85B* | C | — | — | — |
| 85C* | E | — | — | — |
| 85D* | E | — | — | — |
| 86A* | A | B | — | A |
| 86B* | C | — | — | — |
| 86C* | E | — | — | — |
| 86D* | E | — | — | — |
| 87A* | A | B | — | B |
| 87B* | C | — | — | — |
| 87C* | E | — | — | — |
| 87D* | E | — | — | — |
| 88A* | A | A | — | A |
| 88B* | A | — | — | — |
| 88C* | D | — | — | — |
| 88D* | B | — | — | — |
| 89A* | A | A | — | C |
| 89B* | A | B | — | D |
| 89C* | B | — | — | — |
| 89D* | B | — | — | — |
| 90A* | A | A | — | D |
| 90B* | A | B | — | — |
| 90C* | C | — | — | — |
| 90D* | B | — | — | — |
| 91A* | A | — | — | — |
| 91B* | A | — | — | — |
| 91C* | D | — | — | — |
| 91D* | B | — | — | — |
| 92A* | A | A | — | A |
| 92B* | B | — | — | D |
| 92C* | D | — | — | — |
| 92D* | B | — | — | — |
| 93A* | A | B | — | C |
| 93B* | B | — | — | — |
| 93C* | C | — | — | — |
| 93D* | B | — | — | — |
| 94A* | A | B | — | D |
| 94B* | — | — | — | — |
| 94C* | — | — | — | — |
| 94D* | — | — | — | — |
| 95A* | A | B | — | D |
| 95B* | — | — | — | — |
| 95C* | — | — | — | — |
| 95D* | — | — | — | — |
| 96A* | A | C | A | — |
| 96B* | — | — | — | — |
| 96C* | — | — | — | — |
| 96D* | — | — | — | — |
| 97A* | A | C | A | — |
| 97B* | — | — | — | — |
| 97C* | — | — | — | — |
| 97D* | — | — | — | — |
| Rac-98A*/D* | A | B | — | B |
| Rac-98B*/C* | B | — | — | C |
| 99A* | A | B | — | C |
| 99B* | B | — | — | — |
| 99C* | D | — | — | — |
| 99D* | C | — | — | — |
| 100A* | A | C | — | D |
| 100B* | C | — | — | — |
| 100C* | E | — | — | — |
| 100D* | E | — | — | — |
| 101A | A | — | — | A |
| 101B* | E | — | — | — |
| 101C* | E | — | — | — |
| 101D* | C | — | — | — |
| 102A* | A | — | — | A |
| 102B* | — | — | — | — |
| 102C* | — | — | — | — |
| 102D* | C | — | — | — |
| 103A* | A | — | — | A |
| 103B* | E | — | — | — |
| 103C* | E | — | — | — |
| 103D* | C | — | — | — |
| 104A* | A | — | — | C |
| 104B* | C | — | — | — |
| 104C* | A | — | — | A |
| 104D* | C | — | — | — |
| 104E* | C | — | — | — |
| 104F* | C | — | — | — |
| 104G* | D | — | — | — |
| 104H* | D | — | — | — |
| 105 | — | D | — | — |
| 106 | — | D | — | — |

### Comparative Examples

It has been found compounds of Formula (I-A), which comprise a non-aromatic Ring B, as described herein, provide an improvement in activity compared to compounds with a smaller (5-membered) ring at the corresponding Ring B position. See, e.g., the data in Table C, demonstrating a >40-fold increase in binding activity (SPA IC₅₀) and >6-fold increase in potency as measured by hWB IC₅₀ moving from Comparative Example A* (5-membered ring at the corresponding Ring B position) to Compound 10A (6-membered Ring B). Larger rings, e.g., 7-membered Ring B systems, are also well tolerated; compare, for example, the >4-fold increase in binding activity (SPA IC₅₀), the 48-fold increase in potency as measured by PBMC IC₅₀, and the >10-fold increase in potency as measured by hWB IC₅₀, moving from Compound 10A (6-membered Ring B) to Compound 51A (7-membered Ring B).

It has further been found compounds of Formula (I-A), which comprise a non-aromatic Ring B, as described herein, provide an improvement in activity compared compounds with a phenyl ring at the corresponding Ring B position. See, e.g., the data in Table C, demonstrating a 26-fold increase in potency as measured by mWB IC₅₀ moving from Comparative Example B1 to Compound 10A.

Inclusion of an ortho —OH substituent on Ring A of compounds of Formula (I-A), wherein G₁ is CR^{G1} and R^{G1} is —OH, also demonstrates an improvement in activity. See, e.g., the >5-fold increase in binding activity (SPA IC₅₀), the >10-fold increase in potency as measured by PBMC IC₅₀, and the >13-fold increase in potency as measured by hWB IC₅₀ moving from Compound 10A to Compound 15A by inclusion of an ortho —OH group on Ring A. Also compare the 18-fold increase in binding activity (SPA IC₅₀), the 15-fold increase in potency as measured by PBMC IC₅₀, and the respective 4.9-fold and >11-fold increase in potency (as measured by mWB and hWB IC₅₀) moving from Compound 46A to Compound 47A by inclusion of an ortho —OH group on Ring A.

Inclusion of an ortho halo substituent on Ring A of compounds of Formula (I-A), e.g., for example wherein G₁ is CR^{G1} and R^{G1} is halo (e.g., fluoro), is also well tolerated. See, e.g., the 2.5 increase in potency as measured by PBMC IC₅₀ moving from Compound 10A to Compound 105 by inclusion of an ortho fluoro group on Ring A.

Various other substitutions on Ring A of Formula (I-A) are also well tolerated, e.g., for example wherein R¹ of Ring A is halo, C₁ haloalkyl, or —O(C₁haloalkyl). Compare, for example, the relatively similar binding activity and potency for Compound 47A comprising a para-chloro R¹ substituent on Ring A to Compound 16A comprising a para-trifluoromethyl R¹ substituent and to Compound 17A comprising a para-trifluoromethoxy R¹ substituent.

Inclusion of substitution on Ring B of compounds of Formula (I-A) is also well tolerated, e.g., for example wherein at least one of R^{2a} and R^{2b} of Ring B is C₁ alkyl. See, e.g., the 11-fold increase in binding activity (SPA IC₅₀), the 21-fold increase in potency as measured by PBMC IC₅₀, and the respective 11-fold and 3-fold increase in potency (as measured by mWB and hWB IC₅₀) moving from Compound 10A to Compound 46A by a single methyl substitution at the R^{2a}/R^{2b} position of Ring B. Compare, also, the relatively similar binding activity (SPA IC₅₀) and potency (PBMC IC₅₀) for Compound 47A comprising one methyl substituent (R^{2a} or R^{2b}) to Compound 42A* comprising two methyl (R^{2a} and R^{2b}) substituents.

An ethylene bridging group on Ring B has been found to be also well tolerated; compare, e.g., the 6.6-fold increase in binding activity (SPA IC₅₀), the 10-fold increase in potency as measured by PBMC IC₅₀, and the 3-fold increase in potency as measured by hWB IC₅₀ moving from Compound 46A (with no bridging group) to Compound 80A*. Further compare, e.g., the relatively similar binding activity (SPA IC₅₀) and potency (hWB IC₅₀) moving from Compound 52A (with no bridging group) to Compound 92A*.

Additional structural motifs present in compounds of Formula (I-A) and which have been found to improve activity include:
(i) a free NH amide linker moiety directly attached to Ring B
   a. Compare, e.g., the SPA IC₅₀ data for the N-Methylated Comparative Example B2 to the free NH Comparative Example B1, demonstrating a 25-fold increase in binding activity;
(ii) a spirofused cyclopropyl group at the benzylic carbon attached to Ring A
   a. Compare, e.g., the PBMC IC₅₀ data for Comparative Example G, Comparative Example J1 and Comparative Example J2, comprising no benzylic carbon to Compound 10A comprising a spirofused cyclopropyl group, demonstrating a >47-fold increase in potency for each comparison;
   b. Compare, e.g., the PBMC IC₅₀ data for Comparative Example H, comprising an unsubstituted benzylic carbon to Compound 10A comprising a spirofused cyclopropyl group, demonstrating a 5-fold increase in potency;
(iii) the presence of an unsubstituted tetrazolyl substituent attached to the Ring B nitrogen ring atom
   a. Compare, e.g., the SPA IC₅₀ data for Comparative Example D, comprising a corresponding phenyl ring, to Comparative Example B1, comprising an unsubstituted tetrazolyl ring, demonstrating a 40-fold increase in binding activity;
   b. Compare, e.g., the SPA IC₅₀ data for Comparative Example E1 and Comparative Example E2, comprising N-methylated tetrazolyl rings, to Comparative Example B1, comprising an unsubstituted tetrazolyl ring, demonstrating a >60-fold increase in binding activity for each comparison; and
   c. Compare, e.g., the PBMC IC₅₀ data for Comparative Example F, comprising an N-methylated tetrazole ring, to Compound 10A, comprising an unsubstituted tetrazolyl ring, demonstrating a 4-fold increase in potency.

TABLE C
Comparative data

| | SPA IC₅₀ | PBMC IC₅₀ | mWB IC₅₀ | hWB IC50 |
|---|---|---|---|---|
| Compound | (uM) | (uM) | (uM) | (uM) |
| 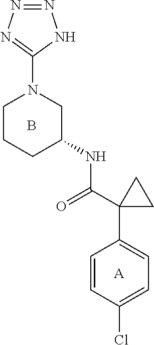 Compound 10A (R)-N-(1-(1H-tetrazol-5-yl)piperidin-3-yl)- 1-(4-chlorophenyl)cyclopropane-1- carboxamide | 9.5 | 2.1 | 1.9 | 7.3 |
| 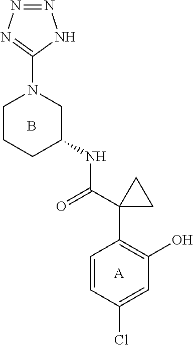 Compound 15A (R)-N-(1-(1H-tetrazol-5-yl)piperidin-3-yl)- 1-(4-chloro-2-hydroxyphenyl)cyclopropane- 1-carboxamide | 1.8 | 0.2 | — | 0.54 |
| 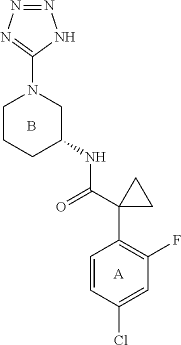 Compound 105 (R)-N-(1-(1H-tetrazol-5-yl)piperidin-3-yl)- 1-(4-chloro-2-fluorophenyl)cyclopropane-1- carboxamide | — | 0.85 | — | — |
| 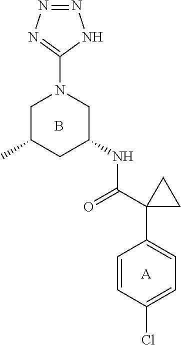 Compound 46A 1-(4-chlorophenyl)-N-((3R,5S)-5-methyl-1- (1H-tetrazol-5-yl)piperidin-3- yl)cyclopropane-1-carboxamide | 0.86 | 0.10 | 0.17 | 2.5 |
| 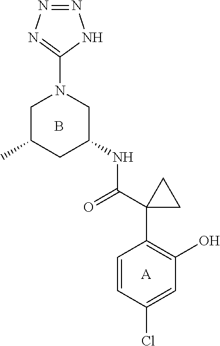 Compound 47A 1-(4-chloro-2-hydroxyphenyl)-N-((3R,5S)- 5-methyl-1-(1H-tetrazol-5-yl)piperidin-3- yl)cyclopropane-1-carboxamide | 0.048 | 0.0067 | 0.035 | 0.22 |
| 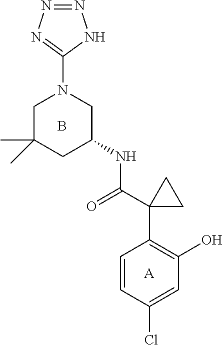 Compound 42A* (R)-1-(4-chloro-2-hydroxyphenyl)-N-(5,5- dimethyl-1-(1H-tetrazol-5-yl)piperidin-3- yl)cyclopropane-1-carboxamide | 0.047 | 0.0058 | — | — |
| 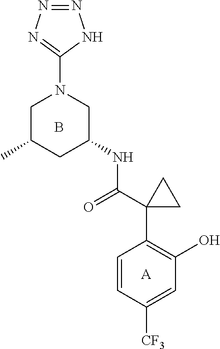 Compound 16A 1-(2-hydroxy-4-(trifluoromethyl)phenyl)-N- ((3R,5S)-5-methyl-1-(1H-tetrazol-5- yl)piperidin-3-yl)cyclopropane-1- carboxamide | 0.041 | 0.0043 | 0.082 | 0.061 |
| 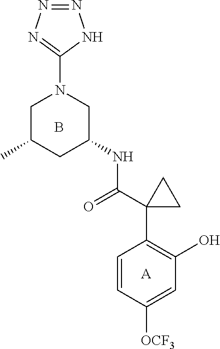 Compound 17A 1-(2-hydroxy-4-(trifluoromethoxy)phenyl)- N-((3R,5S)-5-methyl-1-(1H-tetrazol-5- yl)piperidin-3-yl)cyclopropane-1- carboxamide | 0.098 | 0.017 | — | 0.23 |
| 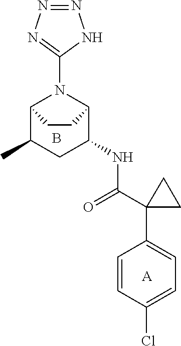 Compound 80A* 1-(4-chlorophenyl)-N-((1R,2R,4R,5S)-4- methyl-8-(1H-tetrazol-5-yl)-8- azabicyclo[3.2.1]octan-2-yl)cyclopropane- 1-carboxamide | 0.13 | 0.010 | — | 0.84 |
| 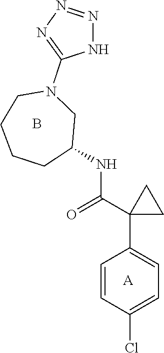 Compound 51A 1-(4-chlorophenyl)-N-[(3R)-1-(1H-tetrazol- 5-yl)azepan-3-yl]cyclopropanecarboxamide | 2.2 | 0.044 | 1.8 | 0.72 |
| 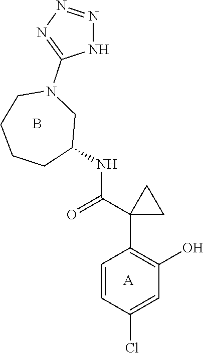 Compound 52A (R)-N-(1-(1H-tetrazol-5-yl)azepan-3-yl)-1- (4-chloro-2-hydroxyphenyl)cyclopropane-1- carboxamide | 0.079 | — | — | 0.26 |
| 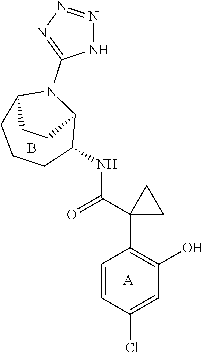 Compound 92A* N-((1R,2R,6R)-9-(1H-tetrazol-5-yl)-9- azabicyclo[4.2.1]nonan-2-yl)-1-(4-chloro-2- hydroxyphenyl)cyclopropane-1- carboxamide | 0.06 | 0.0032 | — | 0.41 |
| 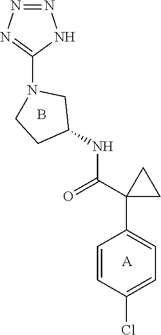 Comparative Example A* (R)-N-(1-(1H-tetrazol-5-yl)pyrrolidin-3-yl)- 1-(4-chlorophenyl)cyclopropane-1- carboxamide | 420 | — | — | >50 |
| 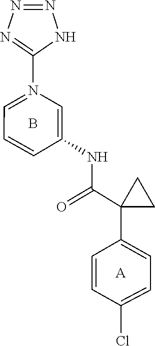 Comparative Example B1 N-(3-(1H-tetrazol-5-yl)phenyl)-1-(4- chlorophenyl)cyclopropane-1-carboxamide CAS Reg. No. 1197529-87-5 | 8.0 | 0.48 | >50 | — |
| 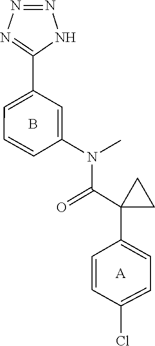 Comparative Example B2 N-(3-(1H-tetrazol-5-yl)phenyl)-1-(4- chlorophenyl)-N-methylcyclopropane-1- carboxamide | 200 | — | — | >50 |
| 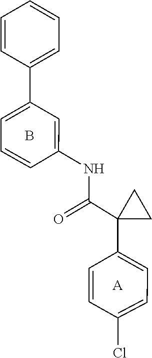 Comparative Example D N-([1,1′-biphenyl]-3-yl)-1-(4- chlorophenyl)cyclopropane-1-carboxamide | 320 | — | — | >50 |
| 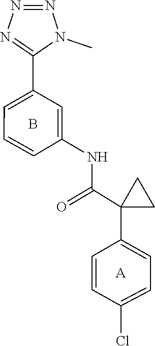 Comparative Example E1 1-(4-chlorophenyl)-N-(3-(1-methyl-1H- tetrazol-5-yl)phenyl)cyclopropane-1- carboxamide | >500 | — | — | — |
| 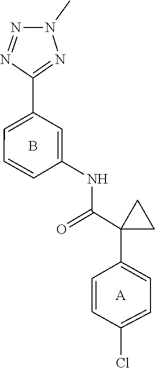 Comparative Example E2 1-(4-chlorophenyl)-N-(3-(2-methyl-2H- tetrazol-5-yl)phenyl)cyclopropane-1- carboxamide | >500 | — | — | — |
| 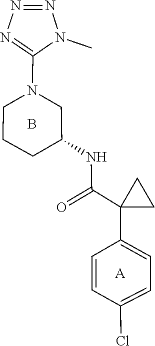 Comparative Example F (R)-1-(4-chlorophenyl)-N-(1-(1-methyl-1H- tetrazol-5-yl)piperidin-3-yl)cyclopropane-1- carboxamide | — | 8.4 | — | — |
| 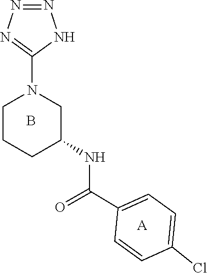 Comparative Example G (R)-N-(1-(1H-tetrazol-5-yl)piperidin-3-yl)- 4-chlorobenzamide | — | >100 | — | — |
| 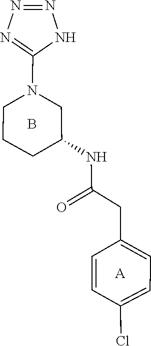 Comparative Example H (R)-N-(1-(1H-tetrazol-5-yl)piperidin-3-yl)- 2-(4-chlorophenyl)acetamide | — | 10.5 | — | — |
| 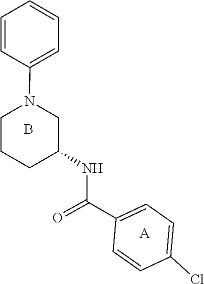 Comparative Example J1 ** (R)-4-chloro-N-(1-phenylpiperidin-3- yl)benzamide | — | >100 | — | — |
| 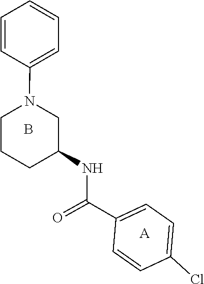 Comparative Example J2 ** (S)-4-chloro-N-(1-phenylpiperidin-3- yl)benzamide | — | >100 | — | — |

** CAS Reg. No. 1625495-15-9 (racemate)

### OTHER EMBODIMENTS

Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims or embodiments is introduced into another claim or embodiment. For example, any claim that is dependent on another claim can be modified to include one or more limitations found in any other claim that is dependent on the same base claim. Where the present disclosure recites elements presented as lists, e.g., in Markush group format, each subgroup of the elements is also disclosed, and any element(s) can be removed from the group. It should be further understood that when any variable (e.g., an R group) is present more than one time in a given Markush structure (e.g., 2 or more times), and that variable may be selected from a given list of two or more elements, unless otherwise stated or understood within the context of the present disclosure, that variable (e.g., the R group) at each repeated occurrence (e.g., 2 or more times) is independent of each other, being independently selected from that given list.

It should also be understood that, in general, where the present disclosure, or aspects of the present disclosure, is/are referred to as comprising particular elements and/or features, certain embodiments of the present disclosure or aspects of the present disclosure also consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are understood to be included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the present disclosure, the present disclosure shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein.

Any particular embodiment of the present disclosure can be excluded from any claim, for any reason, whether or not related to the existence of prior art.

The scope of the present embodiments described herein is not intended to be limited to the above Description, but also includes that as set forth in the appended claims. Those of ordinary skill in the art will appreciate that various changes and modifications to this description may be made without departing from the spirit or scope of the present disclosure, as defined in the following claims.

What is claimed is:

1. A compound of Formula (I-A): 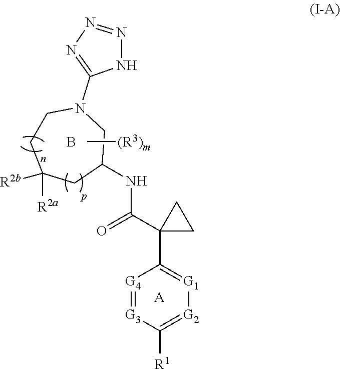
or a pharmaceutically acceptable salt or tautomer thereof;
wherein:
Ring A is a ring system wherein:
G₁ is CR^{G1} or N; G₂ is CR^{G2} or N; G₃ is CR^{G3} or N; and G₄ is CR^{G4} or N; provided no more than two of G₁, G₂, G₃, and G₄ are N;
R¹ is halo, C₁₋₆ alkyl, C₁₋₆haloalkyl, —OR^{G5}, —SR^{G5}, —N(R^{G5})₂, C₃-C₄ carbocyclyl, or 3-4 membered heterocyclyl, wherein the carbocyclyl and heterocyclyl are independently substituted with 0, 1, 2, or 3 halo, C₁₋₆ alkyl, C₁₋₆ haloalkyl, —OR^{G5}, —SR^{G5}, or —N(R^{G5})₂,
or R¹ and G₂, together with the atoms to which they are attached, are joined to form a 5-membered heteroaryl ring independently substituted with 0, 1, 2, or 3 R^{G7};
R^{G1}, R^{G2}, R^{G3}, and R^{G4} are each independently selected from the group consisting of hydrogen, halo, C₁₋₆ alkyl, C₁₋₆ haloalkyl, and —OR^{G6}; and
R^{G5} and R^{G6} are each independently hydrogen, C₁₋₆ alkyl, or C₁₋₆ haloalkyl;
each instance of R^{G7} is independently halo, C₁₋₆ alkyl, C₁₋₆ haloalkyl, —OR^{G5}, —SR^{G5}, and —N(R^{G5})₂; and
Ring B is a ring system wherein:
n is 0 or 1;
p is 1 or 2;
m is 0, 1, 2, or 3;
each instance of R^{2a} and R^{2b} is independently hydrogen, halo, C₁₋₆ alkyl, C₁₋₆ haloalkyl, C₃-C₄ carbocyclyl, or 3-4 membered heterocyclyl, wherein the carbocyclyl or heterocyclyl are each independently substituted with 0, 1, 2, or 3 halo, or R^{2′} and R^{2b} are joined to form a C₃ carbocyclyl independently substituted with 0, 1, 2, or 3 halo; and
each instance of R³ is independently halo, C₁₋₆ alkyl or C₁₋₆ haloalkyl, or two R³ groups are joined to form a C₁₋₃ alkylene bridging group or C₁₋₃ haloalkylene bridging group.

2. The compound of claim 1, wherein the compound is of Formula: 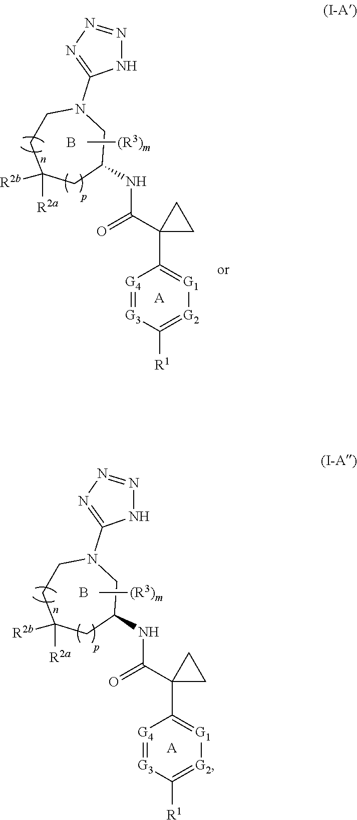
or a pharmaceutically acceptable salt or tautomer thereof.

3. The compound of either claim 1 or claim 2, wherein the compound is of Formula: 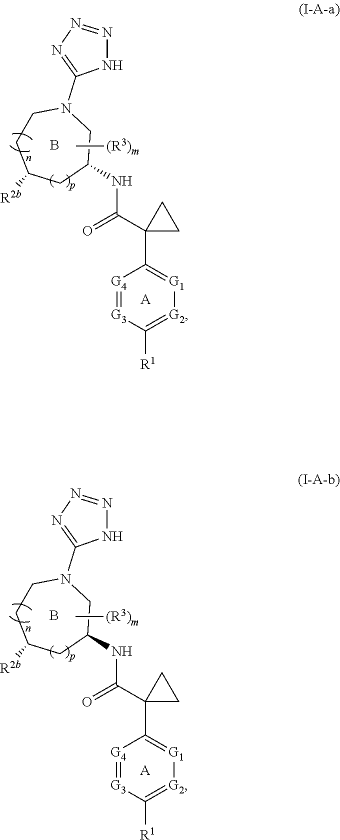 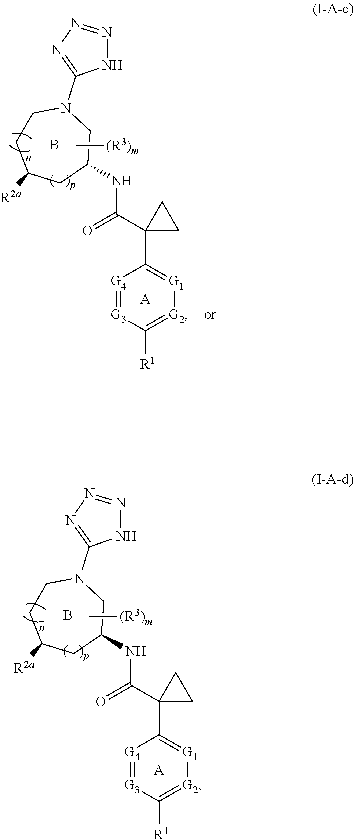
or a pharmaceutically acceptable salt or tautomer thereof.

4. The compound of claim 1, wherein the compound is of the Formula: 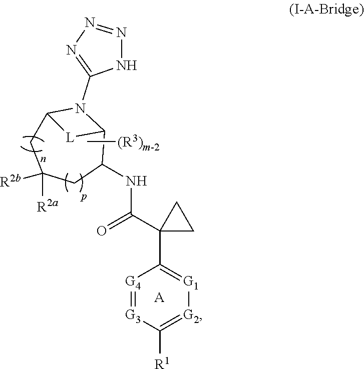
or a pharmaceutically acceptable salt or tautomer thereof, wherein L is a C₁₋₃ alkylene bridging group or a C₁₋₃ haloalkylene bridging group.

5. The compound of claim 4 of the Formula: 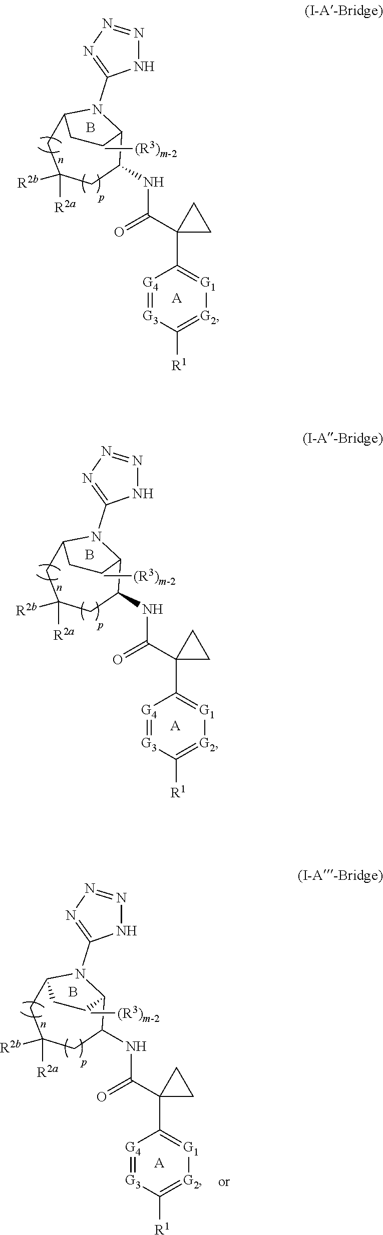 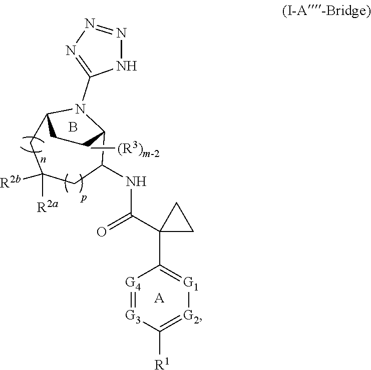
or a pharmaceutically acceptable salt or tautomer thereof, wherein L is a C₁₋₃ alkylene bridging group or a C₁₋₃ haloalkylene bridging group.

6. The compound of claim 4, wherein the compound is of the Formula: 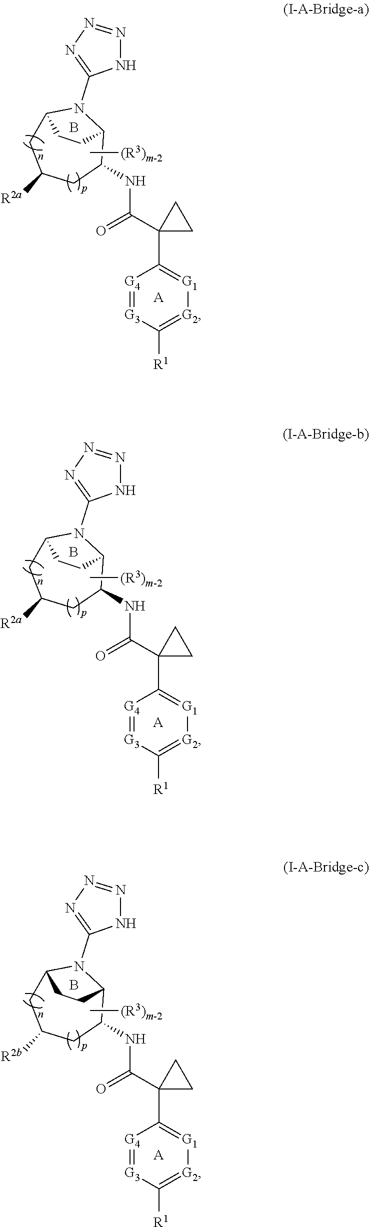 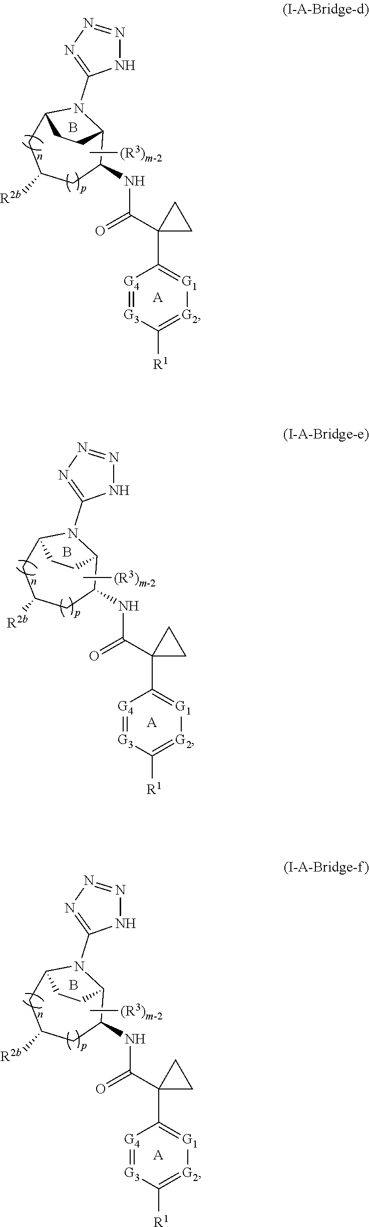 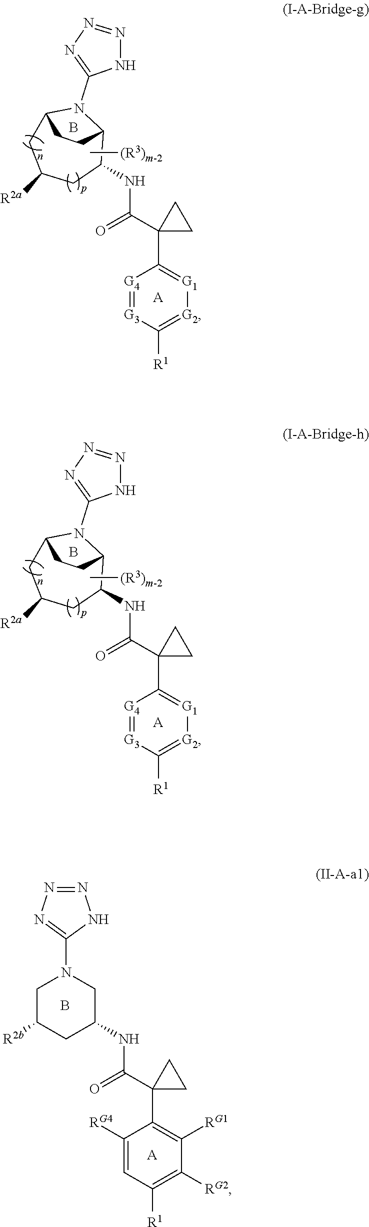 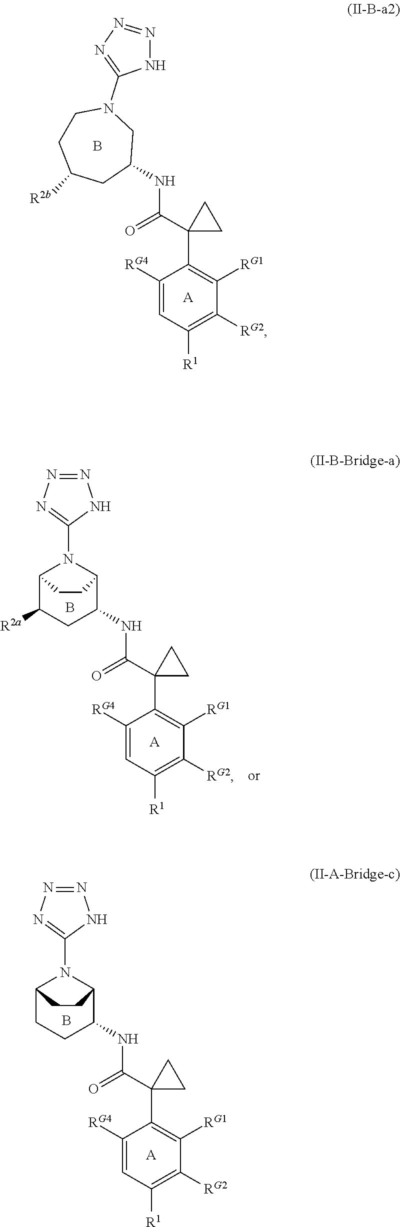
or a pharmaceutically acceptable salt or tautomer thereof.

7. The compound of any one of claims 1-6, or a pharmaceutically acceptable salt or tautomer thereof, wherein Ring A is a ring system wherein:
R¹ is halo, C₁₋₆ alkyl, C₁₋₆ haloalkyl, —OR^{G5}, —SR^{G}, C₃-C₄ carbocyclyl, wherein the carbocyclyl is independently substituted with 0, 1, 2, or 3 halo, C₁₋₆ alkyl, C₁₋₆ haloalkyl, —OR^{G}, —SR^{G5},or —N(R^{G5})₂;
R^{G1}, R^{G2}, R^{G3}, and R^{G4} are each independently selected from the group consisting of hydrogen, halo, C₁₋₆ alkyl, and —OR^{G6}; and
R^{G5} and R^{G6} are each independently hydrogen, C₁₋₆ alkyl, or C₁₋₆ haloalkyl.

8. The compound of claim 7, or a pharmaceutically acceptable salt or tautomer thereof, wherein Ring A is a ring system wherein:
R¹ is Cl, Br, methyl, isopropyl, cyclopropyl, difluorocyclopropyl, cyclobutyl, OCF₂H, OCF₃, CF₂H, CF₃, SCF₃, or SCF₂H; and
R^{G1}, R^{G2}, R^{G3}, and R^{G4} are each independently selected from the group consisting of hydrogen, F, Cl, methyl, OH, OCH₃, and OCF₂H.

9. The compound of any one of claims 1-8, or a pharmaceutically acceptable salt or tautomer thereof, wherein Ring B is a ring system wherein:
each instance of R^{2a} and R^{2b} is independently hydrogen, halo, C₁₋₆ alkyl, C₁₋₆ haloalkyl, or C₃-C₄ carbocyclyl; and
each instance of R³ is independently C₁₋₆ alkyl, or two R³ groups are joined to form a C₁₋₃ alkylene bridging group.

10. The compound of claim 9, or a pharmaceutically acceptable salt or tautomer thereof, wherein Ring B is a ring system wherein:
each instance of R^{2a} and R^{2b} is independently hydrogen, F, CF₃, methyl or cyclopropyl; and
each instance of R³ is independently methyl, or two R³ groups are joined to form an ethylene bridging group.

11. The compound of any one of claims 1-10, or a pharmaceutically acceptable salt or tautomer thereof, wherein:
G₁ is CR^{G1}, G₂ is CR^{G2}, G₃ is CR^{G3}, and G₄ is CR^{G4};
G₁ is CR^{G1}, G₂ is CH, G₃ is CH, and G₄ is CH;
G₁ is CR^{G1}, G₂ is CR^{G2}, G₃ is CH, and G₄ is CH;
G₁ is CR^{G1}, G₂ is CH, G₃ is CR^{G3}, and G₄ is CH;
G₁ is CR^{G1}, G₂ is N, G₃ is CR^{G3}, and G₄ is CR^{G4};
G₁ is CR^{G1}, G₂ is N, G₃ is CH, and G₄ is CH;
G₁ is CR^{G1}, G₂ is CR^{G2}, G₃ is N, and G₄ is CR^{G4};
G₁ is CR⁰¹, G₂ is CH, G₃ is N, and G₄ is CH;
G₁ is CR^{G1}, G₂ is CH, G₃ is N, and G₄ is CH;
G₁ is CR^{G1}, G₂ is CR^{G2}, G₃ is CR^{G3}, and G₄ is N; or
G₁ is CR^{G1}, G₂ is CH, G₃ is CH, and G₄ is N.

12. The compound of claim 11, or a pharmaceutically acceptable salt or tautomer thereof, wherein:
G₁ is CH, G₂ is CH, G₃ is CH, and G₄ is CH; or
G₁ is CH, G₂ is CR^{G2}, G₃ is CH, and G₄ is CH.

13. The compound of any one of claims 1-12, or a pharmaceutically acceptable salt or tautomer thereof, wherein R¹ is Cl, Br, methyl, isopropyl, cyclopropyl, difluorocyclopropyl, cyclobutyl, OCF₂H, OCF₃, CF₂H, CF₃, SCF₃, or SCF₂H.

14. The compound of any one of claims 1-13, or a pharmaceutically acceptable salt or tautomer thereof, wherein R^{G1}, R^{G2}, R^{G3}, and R^{G4} are each independently selected from the group consisting of hydrogen, F, Cl, methyl, OH, OCH₃, and OCF₂H.

15. The compound of any one of claims 1-14, or a pharmaceutically acceptable salt or tautomer thereof, wherein R^{G5} and R^{G6} are each independently hydrogen, C₁₋₆ alkyl, or C₁₋₆ haloalkyl.

16. The compound of claim 15, or a pharmaceutically acceptable salt or tautomer thereof, wherein R^{G5} is CF₂H or CF₃, and R^{G6} is hydrogen, methyl or CF₂H.

17. The compound of any one of claims 1-16, or a pharmaceutically acceptable salt or tautomer thereof, wherein each instance of R^{2a} and R^{2b} is independently hydrogen, halo, C₁₋₆ alkyl, C₁₋₆ haloalkyl, or C₃-C₄ carbocyclyl.

18. The compound of claim 17, or a pharmaceutically acceptable salt or tautomer thereof, wherein each instance of R^{2a} and R^{2b} is independently hydrogen, F, CF₃, methyl or cyclopropyl.

19. The compound of any one of claims 1-18, or a pharmaceutically acceptable salt or tautomer thereof, wherein each instance of R³ is independently C₁₋₆ alkyl.

20. The compound of claim 19, or a pharmaceutically acceptable salt or tautomer thereof, wherein each instance of R³ is independently methyl.

21. The compound of any one of claims 1-20, or a pharmaceutically acceptable salt or tautomer thereof, wherein two R³ groups are joined to form an ethylene bridging group.

22. The compound of any one of claims 1-21, or a pharmaceutically acceptable salt or tautomer thereof, wherein Ring A of formula: 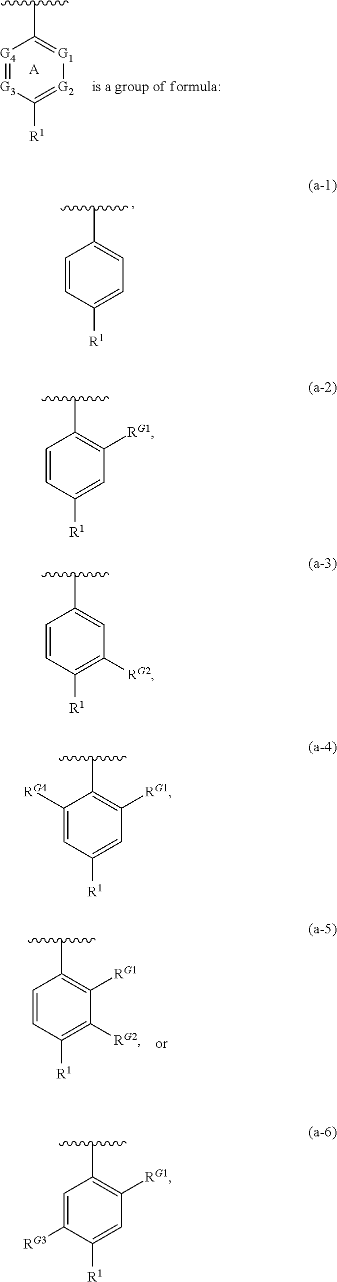
wherein R^{G1}, R^{G2}, R^{G3}, and R^{G4} are each independently selected from the group consisting of halo, C₁₋₆ alkyl, C₁₋₆ haloalkyl, and —OR^{G6}.

23. The compound of claim 22, or a pharmaceutically acceptable salt or tautomer thereof, wherein Ring A of formula (a-2), (a-4), (a-5), or (a-6) is a group of formula:

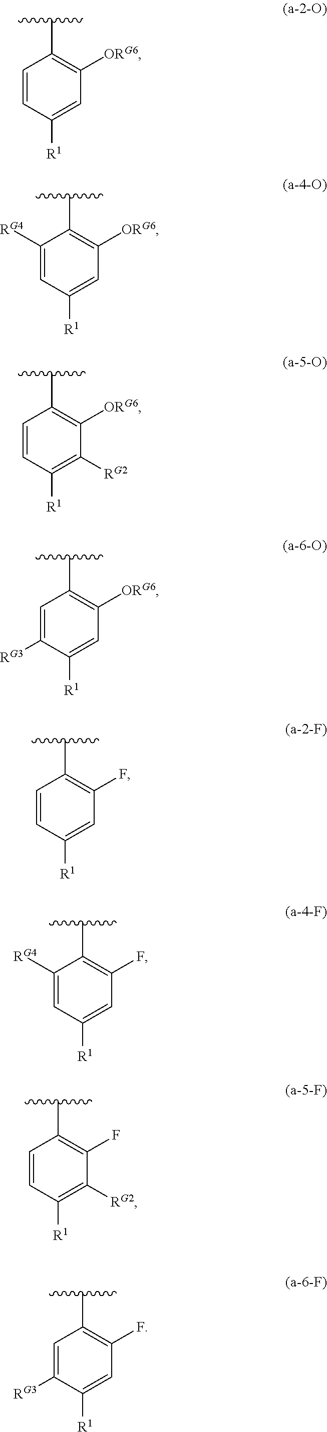

24. The compound of any one of claims 1-23, or a pharmaceutically acceptable salt or tautomer thereof, wherein Ring A of formula: 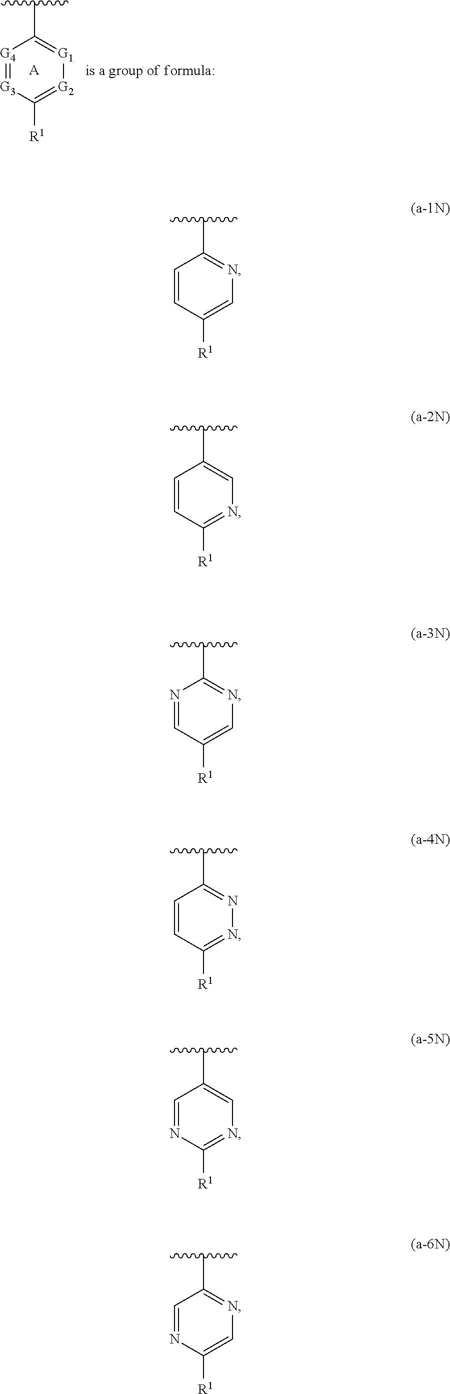 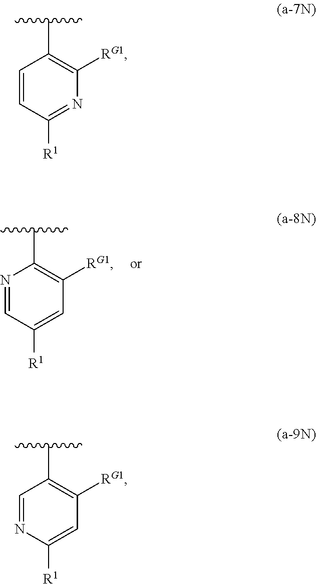
wherein R^{G1} is halo, C₁₋₆ alkyl, C₁₋₆ haloalkyl, or —OR^{G6}.

25. The compound of claim 24, or a pharmaceutically acceptable salt or tautomer thereof, wherein Ring A of formula (a-7N), (a-8N), or (a-9N) is of the formula:

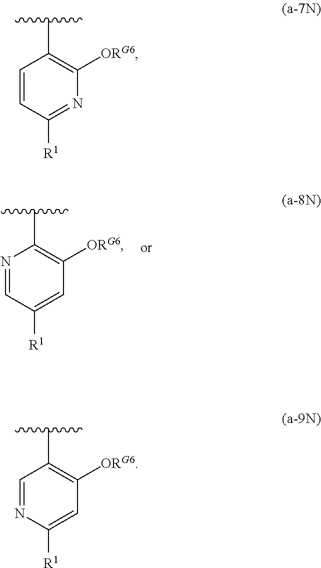

26. The compound of any one of claims 1-21, or a pharmaceutically acceptable salt or tautomer thereof, wherein Ring A of formula: 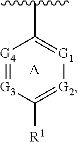
and R¹ and G₂, together with the atoms to which they are attached, are joined to form a 5-membered heteroaryl ring, wherein the Ring A, R¹, and G2 provide a group of formula: 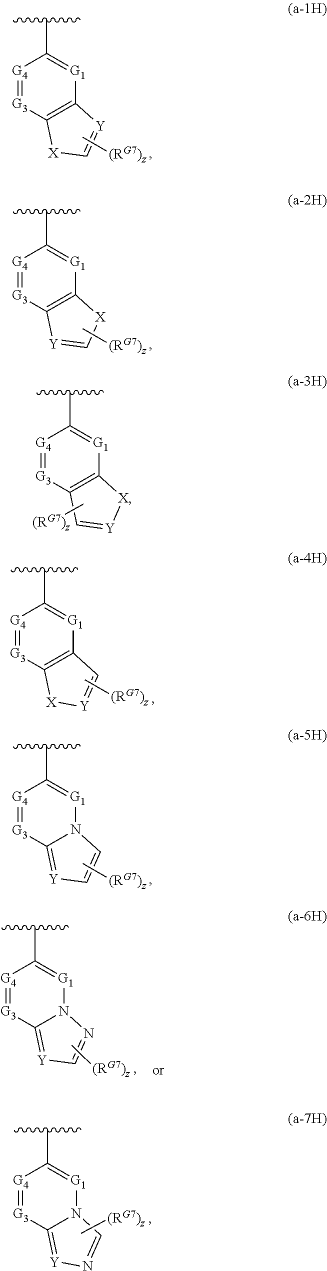
wherein:
X is O, S, NH, or NR^{G7};
Y is N, CH, or CR^{G7}; and
z is 0 or 1;
provided if R^{G7} is a group attached to a nitrogen (N) atom, then R^{G7} is C₁₋₆ alkyl or C₁₋₆ haloalkyl.

27. The compound of claim 26, or a pharmaceutically acceptable salt or tautomer thereof, wherein Ring A, when R¹ and G₂, together with the atoms to which they are attached, are joined to form a 5-membered heteroaryl ring, is a group of formula:

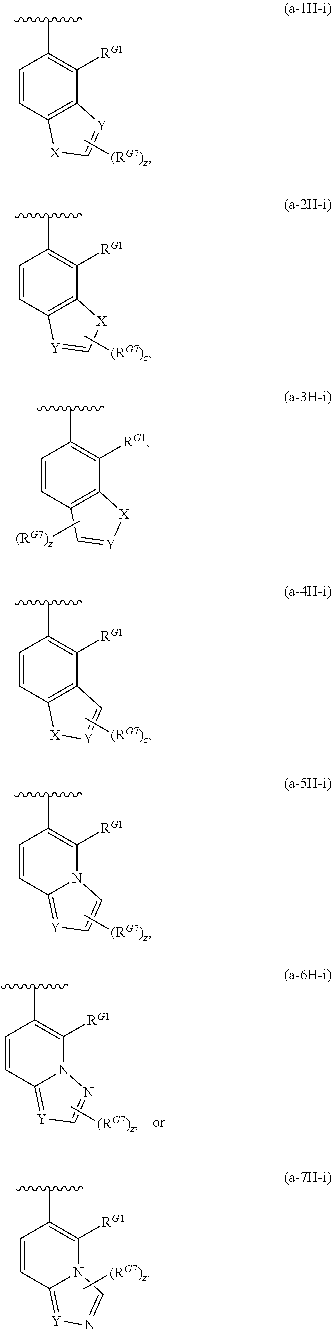

28. The compound of any one of claims 1-27, or a pharmaceutically acceptable salt or tautomer thereof, wherein Ring A is a group of formula:

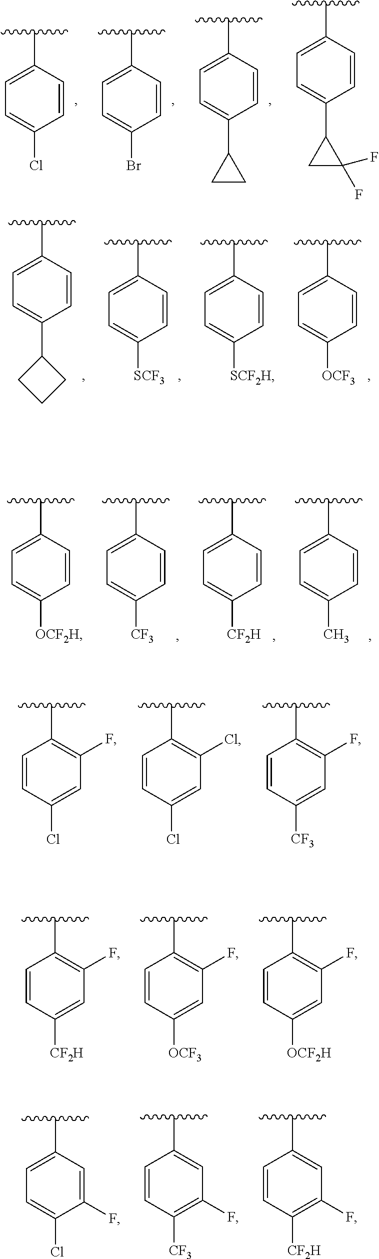

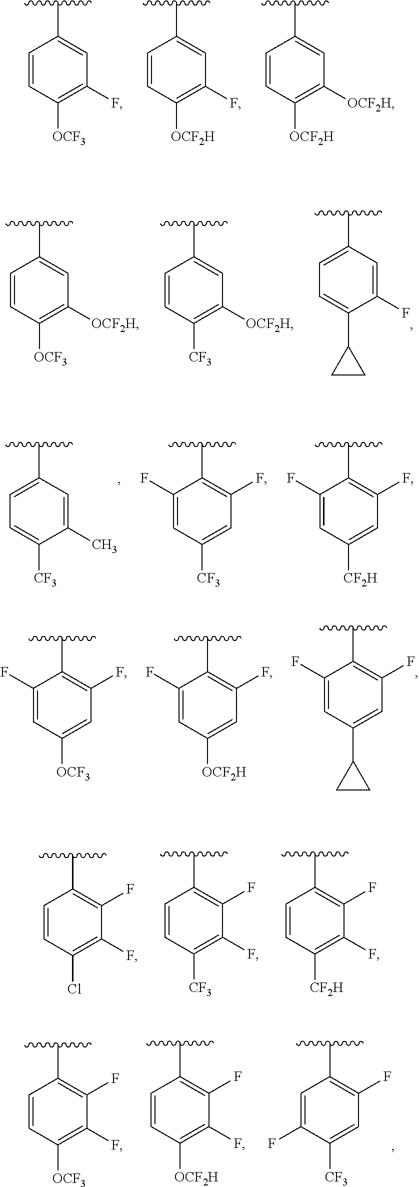

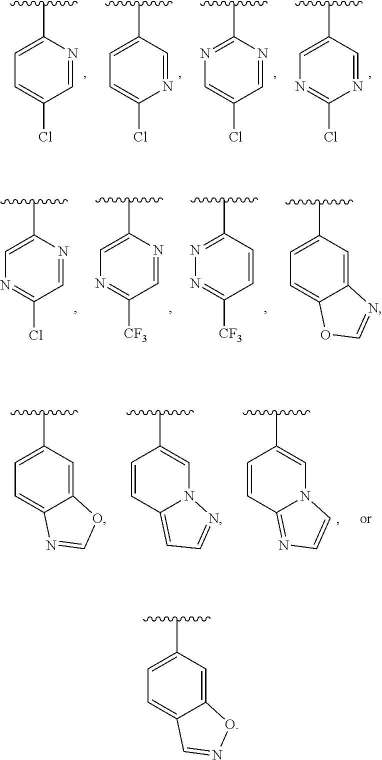

29. The compound of any one of claims 1-28, or a pharmaceutically acceptable salt or tautomer thereof, wherein Ring A is a group of formula:

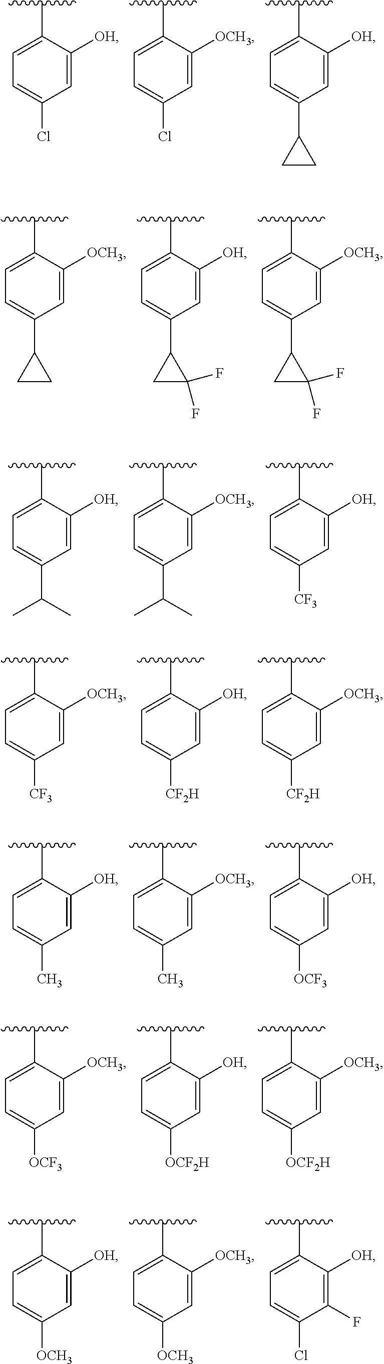

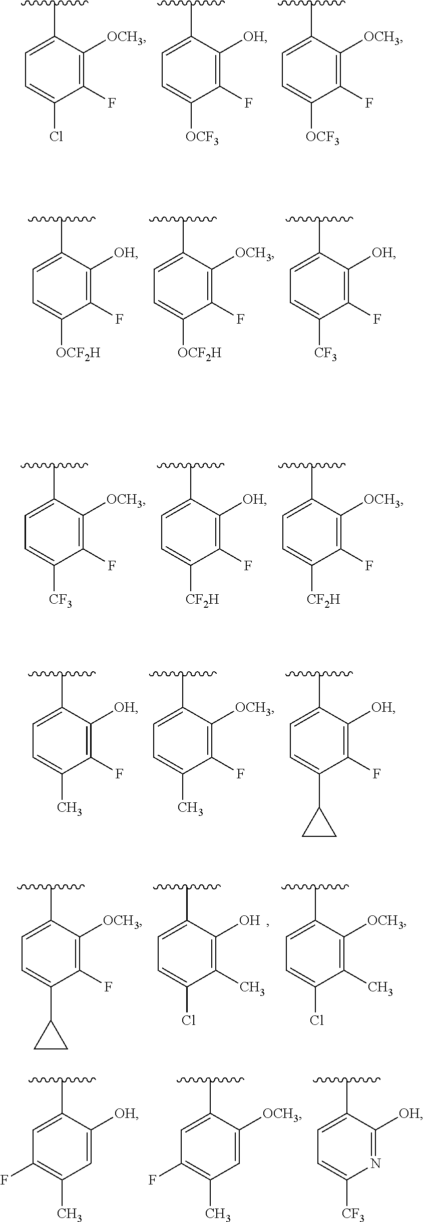

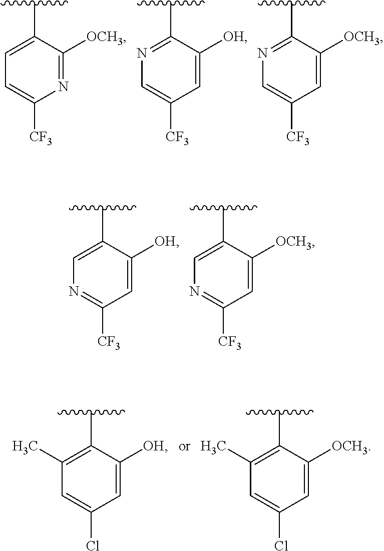

30. The compound of any one of claims 1-29, or a pharmaceutically acceptable salt or tautomer thereof, wherein Ring B of formula: 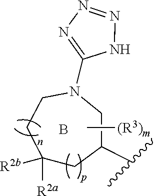
is a group of formula:

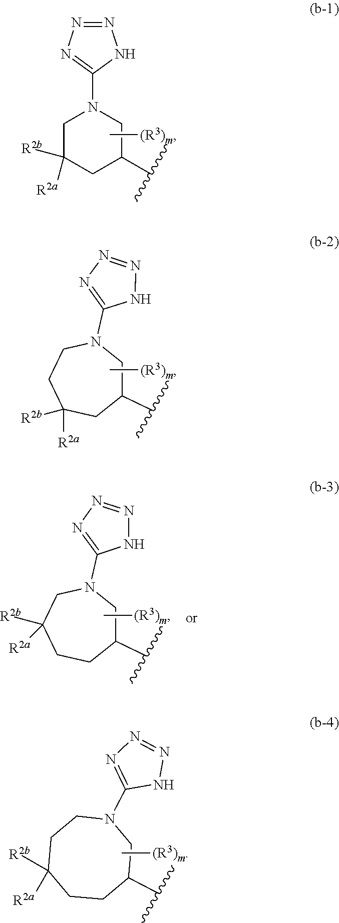

31. The compound of claim 30, or a pharmaceutically acceptable salt or tautomer thereof, wherein Ring B is a group of formula: 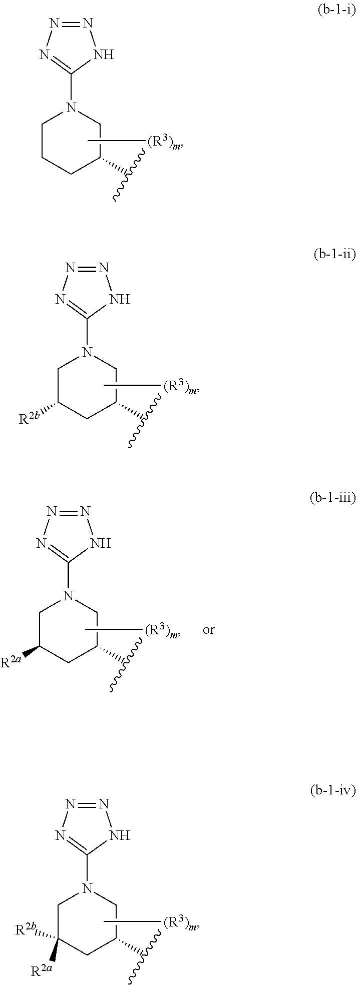
wherein each instance of R^{2a} and R^{2b} is independently halo, C₁₋₆ alkyl, C₁₋₆haloalkyl, C₃-C₄ carbocyclyl, or 3-4 membered heterocyclyl.

32. The compound of claim 30, or a pharmaceutically acceptable salt or tautomer thereof, wherein Ring B of formula (b-1), (b-2), (b-3), or (b-4), when two R³ groups are joined to form a C₁₋₃ alkylene bridging group or C₁₋₃ haloalkylene bridging group, is a group of formula: 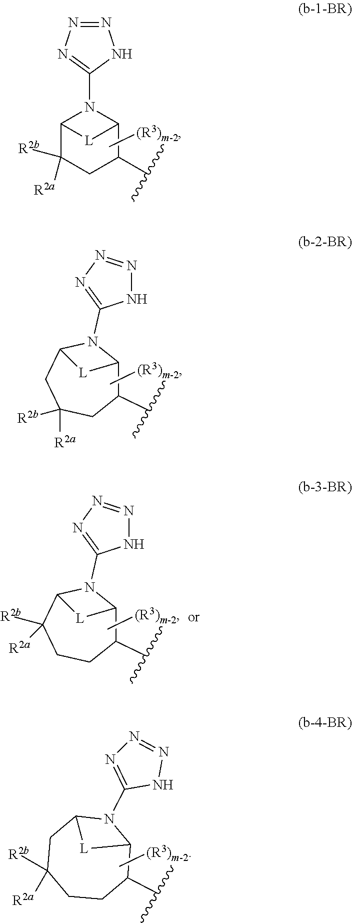
wherein L is a C₁₋₃ alkylene bridging group or C₁₋₃ haloalkylene bridging group.

33. The compound of claim 32, or a pharmaceutically acceptable salt or tautomer thereof, wherein Ring B is a group of formula: 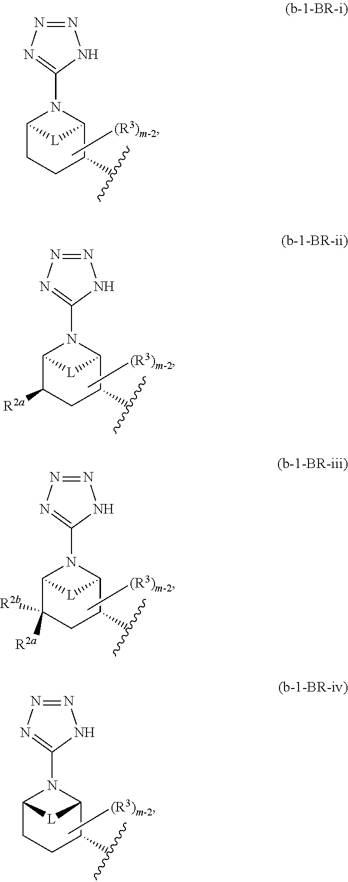
wherein each instance of R^{2a} and R^{2b} is independently halo, C₁₋₆ alkyl, C₁₋₆ haloalkyl, C₃-C₄ carbocyclyl, or 3-4 membered heterocyclyl.

34. The compound of any one of claims 1-33, or a pharmaceutically acceptable salt or tautomer thereof, wherein Ring B is a group of formula:

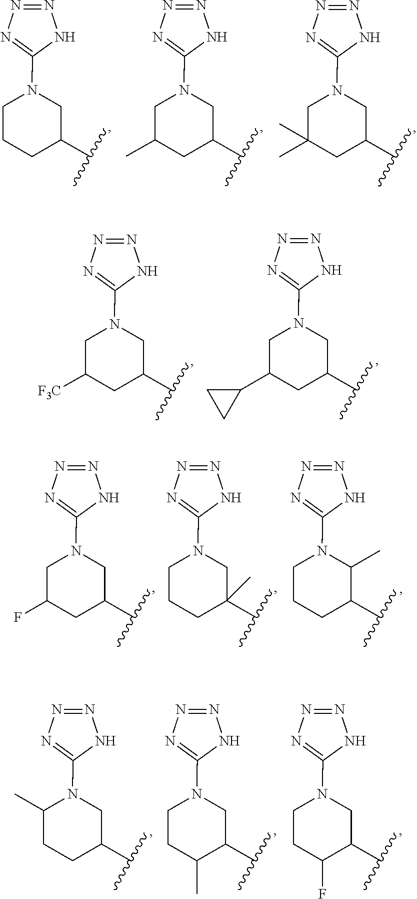

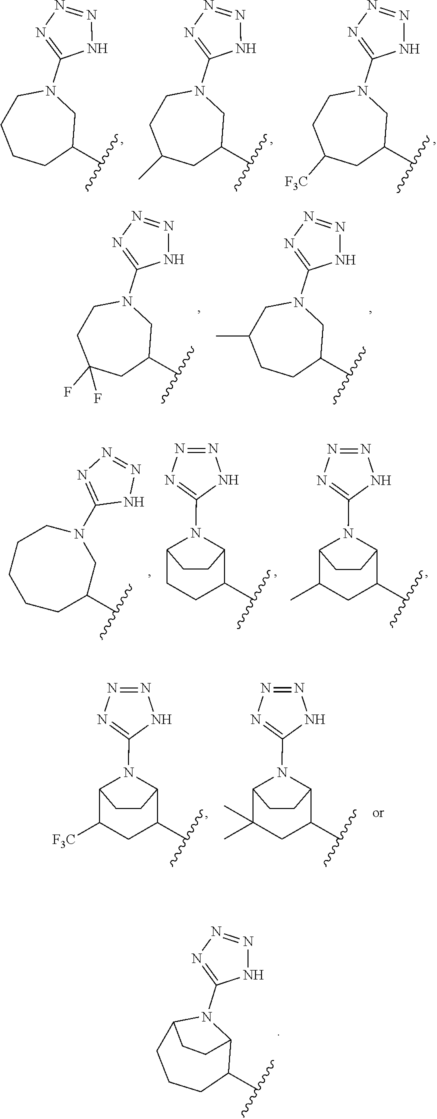

35. The compound of any one of claims 1-34, or a pharmaceutically acceptable salt or tautomer thereof, wherein Ring B is a group of formula:

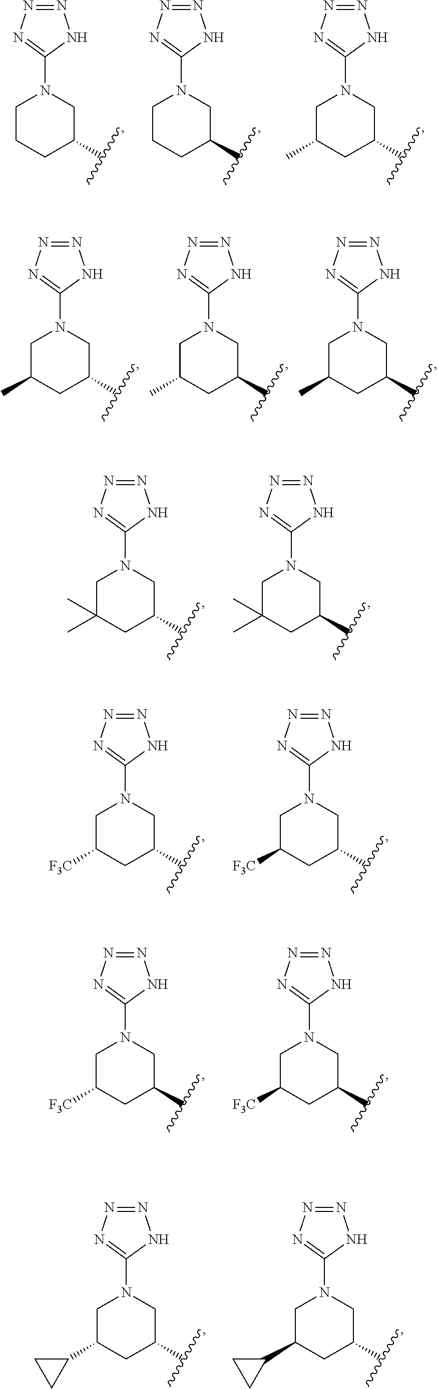

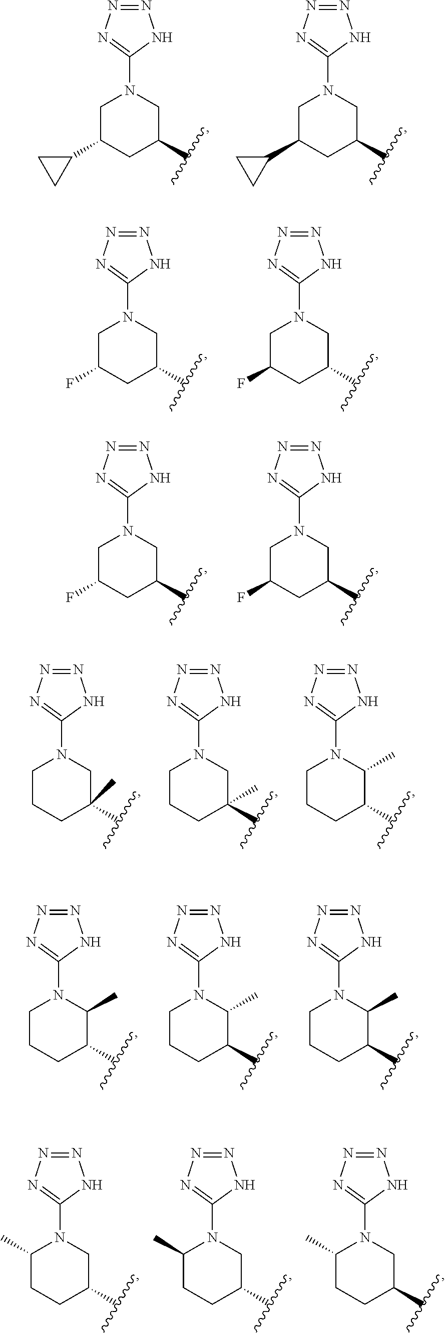

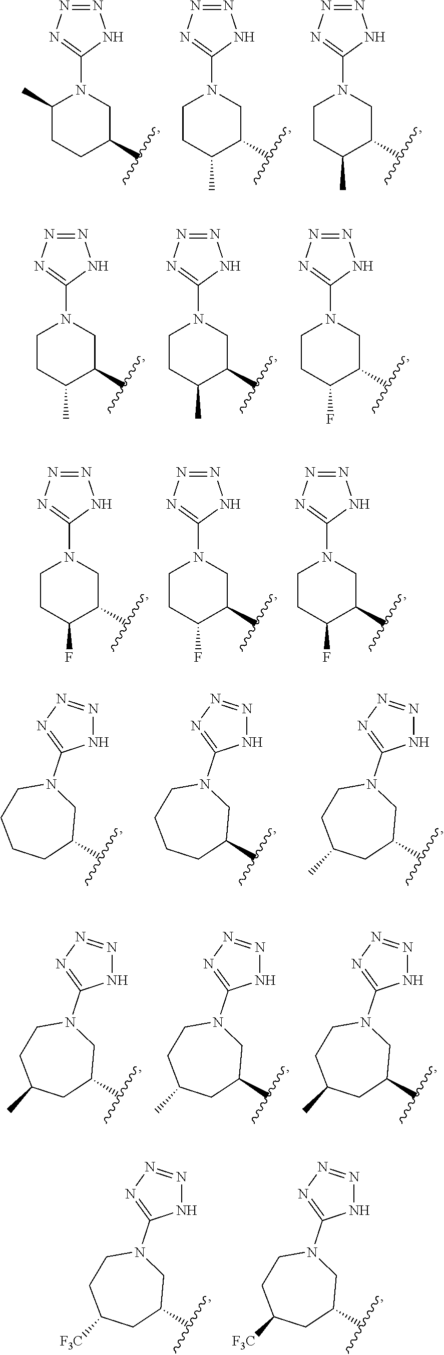

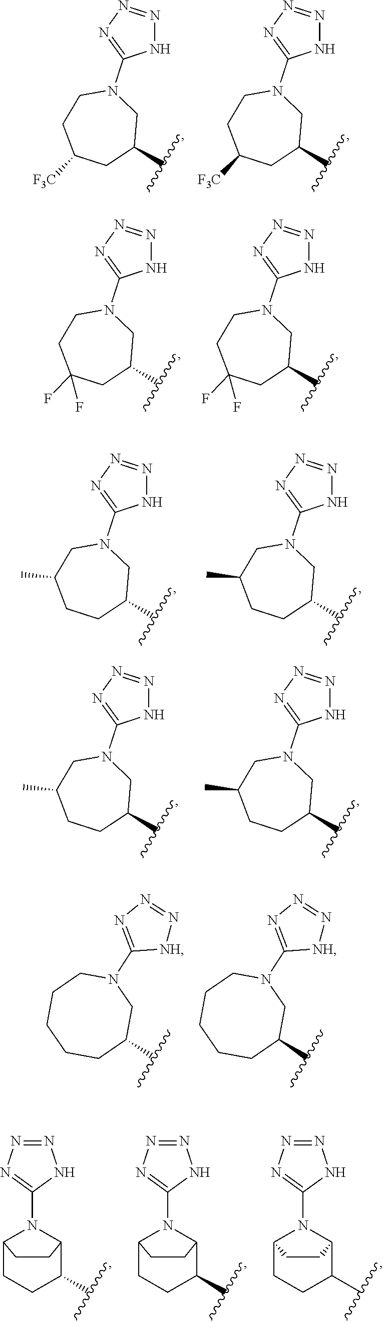

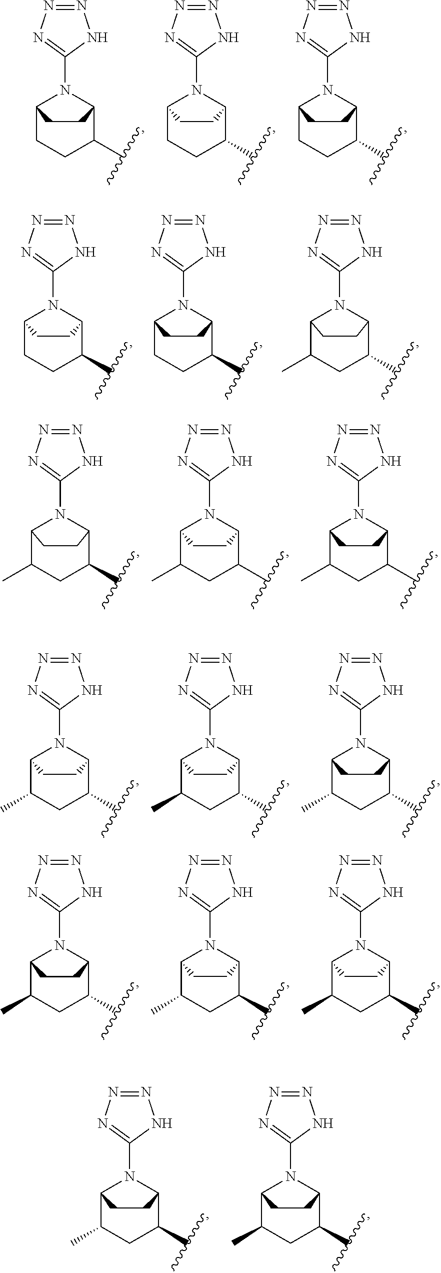

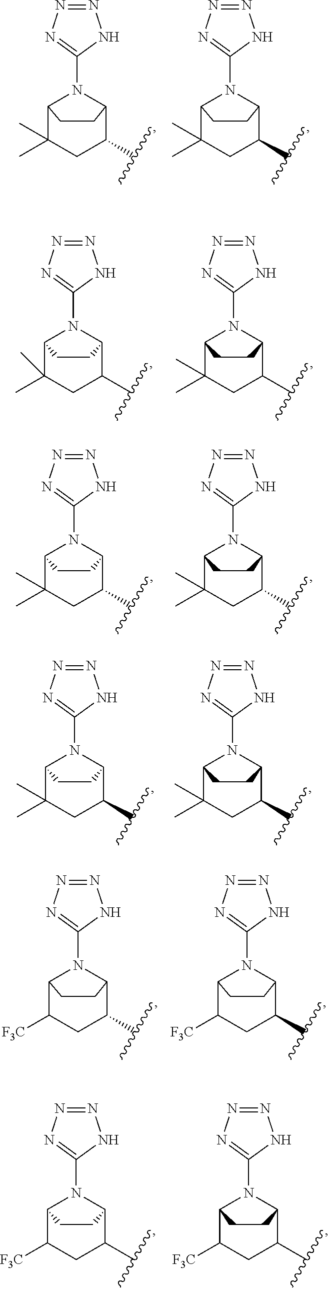

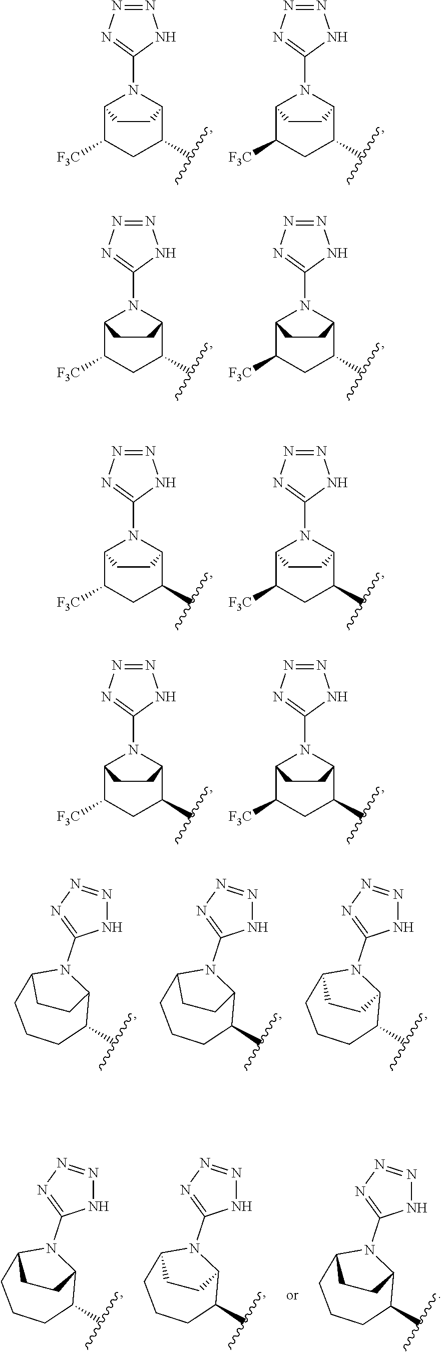

36. The compound of any one of the preceding claims, wherein the compound is selected from the compounds described in Tables 1, 2, or 3, or a pharmaceutically acceptable salt or tautomer thereof.

37. A pharmaceutical composition comprising the compound of any one of claims 1-36, or a pharmaceutically acceptable salt or tautomer thereof, and one or more pharmaceutically acceptable carriers.

38. A method of modulating NLRP3, the method comprising administering to the subject a compound of any one of claims 1-36, or a pharmaceutically acceptable salt or tautomer thereof, or a pharmaceutical composition of claim 37.

39. A method of treating or preventing a disease or disorder, the method comprising administering to the subject a compound of any one of claims 1-36, or a pharmaceutically acceptable salt or tautomer thereof, or a pharmaceutical composition of claim 37.

40. The compound of any one of claims 1-36, or a pharmaceutically acceptable salt or tautomer thereof, or a pharmaceutical composition of claim 37, for use in treating or preventing a disease or disorder.

41. Use of the compound of any one of claims 1-36, or a pharmaceutically acceptable salt or tautomer thereof, in the manufacture of a medicament, for the treatment or prevention of a disease or disorder.

42. Use of the compound of any one of claims 1-36, or a pharmaceutically acceptable salt or tautomer thereof, for the treatment or prevention of a disease or disorder.

43. The method, compound, or use of any one of claims 38-42, wherein the disease or disorder is a disease or disorder of the central nervous system (CNS), a disease or disorder of the peripheral nervous system (PNS), a primary neurological disease of the muscles, an inflammatory disorder, an autoimmune disorder, cancer, an infection, obesity, a metabolic disease, a cardiovascular disease, a respiratory disease, a kidney disease, a liver disease, an ocular disease, a skin disease, a lymphatic disease, a rheumatic disease, a psychological disease, graft versus host disease, pain (including disorders related to pain management), or an NLRP3-related disease in a subject that has been determined to carry a germline or somatic non-silent mutation in NLRP3.

44. A process for preparing a compound of Formula (I-A) of any one of the preceding claims, or a salt or tautomer thereof, wherein the compound is synthesized according to General Schemes A, B, or C.
